(19) United States
(12) Patent Application Publication
(10) Pub. No.: US 2006/0088473 A1
(43) Pub. Date: Apr. 27, 2006

(54) Treatment screening methods

(76) Inventors: Charles Dowding, San Diego, CA (US); James M. Frincke, San Diego, CA (US); Armando Garsd, San Diego, CA (US); Christopher L. Reading, San Diego, CA (US); Dwight R. Stickney, Granite Bay, CA (US); Clarence N. Ahlem, San Diego, CA (US)

Correspondence Address: HOLLIS-EDEN PHARMACEUTICALS, INC., 4435 EASTGATE MALL, SUITE 400, SAN DIEGO, CA, 92121, US

(21) Appl. No.: 11/242,547

(22) Filed: Oct. 3, 2005

Related U.S. Application Data
(60) Provisional application No. 60/615,307, filed on Oct. 1, 2004.
(60) Provisional application No. 60/628,252, filed on Nov. 15, 2004.

(51) Int. Cl. A61K 49/00

(52) U.S. Cl. 424/9.2

(57) ABSTRACT

The invention includes a method to identify a method to enhance survival of a subject such as a non-human primate that has been exposed to a biological insult such as an ionizing radiation dose of about LD_{30/30} or about LD_{50/30} by treating the exposed subject with a test compound an optionally comparing the results to that obtained using control subjects that had been treated with 3β,17β-dihydroxyandrost-5-ene or other disclosed compounds.

### CROSS REFERENCE TO RELATED APPLICATIONS

This application is a continuation-in-part of nonprovisional application Ser. No. (not yet received) filed on Sep. 30, 2005, entitled ‘Treatment Screening Methods’ by James M. Frincke, et al., which is incorporated herein by reference. This application also claims priority from pending U.S. provisional application Ser. No. 60/615,307 filed Oct. 1, 2004 and pending U.S. provisional application Ser. No. 60/628,252 filed Nov. 15, 2004, both of which are incorporated herein by reference.

### FIELD OF THE INVENTION

The invention relates to invention relates to the use markers or conditions such as the duration of febrile severe neutropenia or the time of onset and/or duration of severe thrombocytopenia to gauge a subject's clinical condition after a subject has been exposed to a potentially lethal biological insult. The use of such markers allows assessment of a subject's clinical prognosis and an accurate indirect assessment of a subject's capacity to maintain levels of endogenous steroids such as 3β,17β-dihydroxyandrost-5-ene.

### BACKGROUND

The clinical status of individuals is often difficult to assess, including in situations where there is a significant risk of death. Various clinical parameters have been associated with a range of prognoses for survival, but generally such associations have been reported as anecdotal, subjective or imprecise. To date, widely accepted precise or objective correlates are not generally available and used to guide common clinical or veterinary practice. Surrogate markers for lethality or death in humans and non-human primates have not been described. Research on a number of clinical conditions, biological responses to biological insults and survival prognosis has been described, e.g., A. M. Farese et al., Blood 82:3012-3018 1993, C. A. Cogos et al., J. Infect. Dis. 181:176-180 2000, B. Katja et al., Shock 15:95-100 2001, C. E. Hack et al., Blood 74:1704-1710 1989, C. E. Hack et al., Am. J. Med. 86:20-26 1989, W. H. McBride et al., Radiation Res. 162:1-19 2004, A. B. J. Groeneveld et al., Clinical Immunol. 106:106-115 2003, G. P. Bodey et al., Ann. Internal Med. 64:328-340 1966, T. Calandra et al., Am. J. Med. 91:23-29 1991, A. W. J. Bossinketal., Chest 113:1533-1541 1998, S. A. Dalrymple et al., Infect Immun. 64:3231-3235 1996 and F. Arnalich et al., Infect. Immun. 68:1942-1945 2000.

Endogenous hormone and steroid levels can fluctuate in response to specific events or to normal physiological processes such as growth or aging. See, e.g., K. G. Hobson et al., Circulating Leptin and Cortisol After Burn Injury: Loss of Diurnal Pattern, J. Burn Care Rehabil. 25(6):491-499 2004; T. W. Buchanan et al., Circadian regulation of cortisol after hippocampal damage in humans, Biol Psychiatry 56(9):651-6 2004; F. Hucklebridge et al., The diurnal patterns of the adrenal steroids cortisol and dehydroepiandrosterone (DHEA) in relation to awakening, Psychoneuroendocrinology 30:51-57 2005; K. Carlstrom et al., Diurnal rhythm and effects of oral contraceptives on serum dehydroepiandrosterone sulfate (DHEAS) are related to alterations in serum albumin rather than to changes in adrenocortical steroid secretion, Scand. J. Clin. Lab. Invest.62(5):361-368 2002; G. Valenti, Neuroendocrine hypothesis of aging: the role of corticoadrenal steroids, J. Endocrinol. Invest. 27 Suppl(6):62-63 2004; Z. Barrou et al., Dehydroepiandrosterone (DHEA) and aging, Arch. Gerontol. Geriatr. 24(3):233-241 1997; A. Tchernof et al., Dehydroepiandrosterone, obesity and cardiovascular disease risk: review of human studies, Eur. J. Endocrinol. 151(1):1-14 2004.

Means and methods to modulate the activity of certain enzymes that mediate steroid metabolism have been described. See, e.g., U.S. Pat. Nos. 6,784,167, 6,541,463, 6,423,698, 6,124,115, 5,817,649, 5,595,985, 5,550,107, 5,439,943, 5,399,790, and 5,118,621. These means and methods have been proposed as treatments for a number of clinical conditions including osteoporosis, breast cancer, prostate cancer, endometriosis, psoriasis and hypercholesterolemia.

### DESCRIPTION OF THE INVENTION

Summary of invention embodiments. Invention embodiments provide a method to identify a treatment method useful to increase the rate or probability of survival of an injured human or non-human primate, comprising (a) exposing non-human primates to a biological insult of at least about an LD_{40/30} to obtain exposed subjects and conducting a treatment protocol obtain exposed treated subjects, wherein the exposed treated subjects are not provided with an ameliorative treatment selected from (i) a transfusion such as a whole blood transfusion(s), a platelet transfusion(s), or an immunoglobulin transfusion(s), (ii) an antimicrobial treatment(s) to treat or prevent an infection, (iii) assisted feeding such as feeding by parenteral or catheter feeding or by tube feeding to the stomach; and (b) determining the survival rate of the exposed treated subjects to obtain a treatment survival rate and comparing the treatment survival rate with a suitable control survival rate that was obtained from exposed subjects that were not provided with any treatment protocol and that were not provided with the ameliorative treatment. In these embodiments, the biological insult can be exposure of the non-human primates to whole body radiation, e.g., about 600 cGy to about 635 cGy.

Other embodiments include a method to determine a status profile for a subject species comprising, (1) exposing subjects to a biological insult of at least about an LD_{40/30} to obtain exposed treated subjects; (2) measuring on two or more occasions in or from the exposed subjects one, two or more biological parameters selected from temperature, circadian rhythm, red blood cell counts, hematocrit, reticulocytes, platelets, megakaryocytes and neutrophils; (3) measuring the survival rate of the exposed subjects; (4) obtaining one or more status profiles that corresponds to a defined probability of surviving the biological insult (Pₛᵤᵣᵥᵢᵥₐₗ) of at least 0.95 or of not surviving the biological insult (P_{lethality}) of at least 0.05; and (5) optionally using the status profile to identify and initiate a profile-based therapy for one or more of the exposed subjects.

In other embodiments, the invention provides a method to (A) maintain approximately normal endogenous levels of 3β,17β-dihydroxyandrost-5-ene and/or 17β-hydroxyandrost-5-ene-3β-sufate in a human or non-human primate that has been or that is anticipated to be exposed to (i) a biological insult of at least about an LD₁₀, (ii) a cytotoxic chemotherapy or (iii) a radiation exposure or (B) decrease the drop in endogenous levels of 3β,17β-dihydroxyandrost-5-ene and/or 17β-hydroxyandrost-5-ene-3β-sufate in human or non-human primate, comprising, administering to the human or non-human primate an effective amount of a modulator compound selected from a steroid hydroxylase inhibitor, a 3-hydroxysteroid dehydrogenase inhibitor, a 17-hydroxysteroid dehydrogenase inhibitor, a steroid sulfotransferase inhibitor and an isoform or isozyme of any of these enzymes.

In vitro and in vivo methods to measure or characterize the activity of enzymes that mediate steroid metabolism, e.g., dehydrogenases, hydroxylases, sulfotransferases, phosphotransferases and glycosylases have been described. See, e.g., T. Kasai et al., J. Clin. Endocrinol. Metab. 89(11):5661-5668 2004; M. Quinkler et al., J. Endocrinol. 183(2):331-42 2004; K. Motojima, Eur. J. Biochem. 271(20):4141-4146 2004; L. J. Martin et al., Endocrinology Oct. 21, 2004 electronic publication ahead of print; A. H. Payne et al., Endocr Rev. Sep. 8, 2004, electronic publication ahead of print; F. Xiao et al., Hypertension 44(3):340-345 2004; R. R. Ugale et al., Brain Res. 1023(1):102-111 2004; S. Steckelbroeck et al., Mol. Pharmacol. Sep. 21, 2004, electronic publication ahead of print, M. H. Bassett et al., J. Mol. Endocrinol. 28(2):125-135 2002; C. Keshava et al., Am. J. Epidemiol. 160(9):825-841 2004; T. Uno et al., Biochemistry. 36(11):3193-3198 1997; R. L. Walsky et al., Drug Metab Dispos. 32(6):647-660 2004. These methods can be used to characterize the effects of the modulator compounds on the levels or activity of such enzymes.

The invention includes a method to (i) increase the survival rate or probability of survival or (ii) enhance the survival experience of a subject that has been exposed to a biological insult such as a radiation dose of at least about an LD_{5/30} or LD_{5/60}, by treating the exposed subject with a modulator compound described herein and optionally with an effective amount of a formula 1 compound described herein such as 3β,17β-dihydroxyandrost-5-ene. Aspects of the invention include treatment of human or non-human primate subjects under conditions where other ameliorative or palliative treatments, e.g., use of painkillers, blood or blood product transfusions or antibiotic treatments such as ciprofloxacin, are not also used.

Methods for the use of one, two three or more surrogate markers such as (i) the duration of febrile severe neutropenia (also referred to as “severe febrile neutropenia”) or the duration of severe neutropenia, (ii) duration of severe thrombocytopenia, (iii) time, e.g., delay, of onset of febrile severe neutropenia or severe neutropenia, (iv) time, e.g., delay, of onset of severe thrombocytopenia, (v) degree of severity of febrile severe neutropenia or severe neutropenia, or (vi) degree of severity of severe neutropenia for lethality or death are described for subjects such as humans, non-human primates or other subjects that have been exposed to a biological insult as described herein. Thus, a subject that has been exposed to a biological insult, e.g., ionizing radiation, of at least about an LD_{10/30} or at least about an LD_{30/30} or at least about an LD_{35/30} or at least about an LD_{40/30} or at least about an LD_{45/30} or at least about an LD_{50/30} or at least about an LD_{60/30} or at least about an LD_{80/30} another biological insult as described herein can be monitored for the time of onset of febrile severe neutropenia, severe neutropenia or severe thrombocytopenia. Survival of the subject can be assessed at any convenient time, e.g., at about 3 weeks, about 4 weeks, about 30 days, about 45 days or about 60 days. Treatment of such exposed subjects with a modulator compound another compound or treatment as described herein can then be assessed for their effect on the onset time of febrile severe neutropenia, severe neutropenia, severe thrombocytopenia or another surrogate marker described herein.

Other invention embodiments are as described elsewhere in the specification including the claims.

Definitions. As used herein and unless otherwise stated or implied by context, terms that are used herein have the meanings defined below. Unless otherwise contraindicated or implied, e.g., by including mutually exclusive elements or options, in these definitions and throughout this specification, the terms “a” and “an” mean one or more and the term “or” means and/or.

“Biological insult” means a treatment or event that is lethal or potentially lethal to a subject. Biological insults include exposure to or treatment with radiation, toxins, trauma, chemotherapy or other events or treatments as disclosed herein.

A “subject” means a human or animal. Usually the animal is a mammal or vertebrate such as a primate, rodent, lagomorph, domestic animal or game animal. Primates include chimpanzees, baboons (Papio), mandrills (Mandrillus), Rhesus monkeys (Macaca mulatta), Cynomolgous monkeys (Macaca fascicularis), Celebes black macaques (Macaca nigra), pig tailed macaques (Macaca nemestrina), bonnet monkey (Macaca radiata), marmosets, spider monkeys and macaques, e.g., Rhesus or Pan. Rodents and lagomorphs include mice, rats, woodchucks, ferrets, rabbits and hamsters. Domestic and game animals include cows, horses, pigs, sheep, deer, bison, buffalo, mink, felines, e.g., domestic cat, canines, e.g., dog, beagle dog, wolf and fox, avian species, e.g., chicken, turkey, emu and ostrich, and fish, e.g., trout, catfish and salmon. Subject includes any subset of the foregoing, e.g., all of the above, but excluding one or more groups or species such as humans, primates or rodents. Other subsets of subjects include subjects of a given species or group of species of varying ages, e.g., young humans, e.g., about 1 week of age to about 9 years of age, adolescent humans, e.g., about 10-19 years of age, adult humans, e.g., about 20-100 years of age, and mature adult or elderly humans, e.g., at least about 55 years of age, at least about 60 years of age, at least about 65 years of age or a range of ages such as about 60-100 years of age. Thus, as used herein, prevention or treatment of a disease, condition or symptom may include or exclude any subset of subjects that are grouped by age. Human subjects include special populations, e.g., young humans and elderly humans.

Reference to an androstane compound, e.g., 3β,16α,17β-trihydroxyandrostane, means that the hydrogen atom at the 5-position is in the α-configuration. For androstanes or androstenes with hydrogen in the β-configuration, the compound name will specify this configuration, e.g., 3β,16α,17β-trihydroxy-5β-androstane.

An “invention formulation”, “formulation”, “pharmaceutical formulation” or the like means a composition that one can administer to a subject, e.g., human, non-human primate, mammal or other animal, without further manipulations that change the ingredients or the ingredient proportions that are present. Formulations will typically comprise a modulator compound and one or more excipients. Formulations are suitable for human or veterinary applications and would typically have expected characteristics for the formulation, e.g., parenteral formulations for human use would usually be sterile and stored in a suitable closed container.

When referring to mixtures that contain a modulator compound means a composition, that is a formulation or that can be an intermediate one can use, e.g., to make a formulation or a formula 1 compound. Compositions also include other types of mixtures, e.g., (1) reagents for assays or cells that are optionally contacted with a formula 1 compound or mixtures of compounds and (2) compounds used to make a formula 1 compound or by-products of formula 1 compound synthesis or analysis.

Phrases such as “administration of a compound of formula 1”, “treatment with a formula 1 compound”, “use of a formula 1 compound” or similar terms mean that the compound(s) is administered to, contacted with or delivered to, the subject or to the subject's cells or tissues in vitro or in vivo by one or more suitable methods, e.g., in vivo delivery can be by an oral, topical, subcutaneous, subdermal, parenteral, buccal or sublingual route.

Expressions such as “a formula 1 compound(s)”, “a formula 1 compound” and the like mean one or more than one formula 1 compound is present, e.g., in a composition, or is used in the disclosed method, typically 1, 2, 3 or 4, usually 1. Any reference to a “formula 1 compound”, “one or more compounds of formula 1” or the like means that the formula 1 compound can have the formula 2 structure or any other structure disclosed herein that is within the definition of formula 1 compounds. The phrase formula 1 compound or formula 1 compound(s) is sometimes abbreviated as “F1C” or “F1C(s)” and formula 1 compounds is usually abbreviated as “F1Cs”.

Reference to subject matter “as disclosed herein” such as a “therapeutic treatment or agent as disclosed herein”, a “dosing protocol as disclosed herein” or a “clinical condition or symptom as disclosed herein” or the like means a treatment, agent, protocol, condition, symptom or the like that is described herein or in any reference that is cited herein.

An “excipient”, “carrier”, “pharmaceutically acceptable excipient”, “pharmaceutically acceptable carrier” or similar terms mean one or more component(s) or ingredient(s) that is acceptable in the sense of being compatible with the modulator compound, the F1C or other ingredients of formulations and not overly deleterious to the patient, animal, tissues or cells to which the modulator compound, F1C, composition or formulation is to be administered.

The terms “effective amount”, “effective dose” or the like with reference to the treatments described herein or to a F1C(s) mean an amount of the treatment or the F1C(s) that is sufficient to elicit a desired response, e.g., detectable amelioration of a clinical condition or symptom.

Terms such as “use”, “treat”, “treatment”, “address” or the like in the context of practicing the methods or using therapeutic agents or using the F1Cs in the treatment methods or other methods disclosed herein mean that the method is practiced or a F1C is administered to a subject, delivered to the subject's tissues or contacted with tissues, cells or cell free systems in vivo or in vitro, e.g., as described herein or a reference cited herein. Typically such use or treatment results in, e.g., (1) detectable improvement in or amelioration of the condition or symptom being treated, (2) detectable modulation in the activity, level or numbers of a relevant biomolecule, therapeutic immune cell population or a pathological cell population, (3) slowing of the progression of a condition or delaying its onset, or reduction of the severity of a symptom(s) of the condition or (4) another detectable response as described herein. Any such amelioration may be transient, e.g., lasting for at least a few, e.g., about 1 to 24, hours or days, e.g., about 1, 2, 3, 4, 5, 6 or 7 days, or amelioration may be prolonged, e.g., lasting about 8, 9, 10, 11, 12, 13, 14, 15, 16, 17, 18, 19, 20, 21, 22, 24, 26, 28, 35, 42, 49, 56 to about 60 days or more, or amelioration may be permanent.

“Ameliorate”, “amelioration”, “improvement” or the like means a detectable improvement or a detectable change consistent with improvement occurs in a subject or in at least a minority of subjects, e.g., in at least about 2%, 5%, 10%, 15%, 20%, 25%, 30%, 40%, 50%, 60%, 70%, 75%, 80%, 85%, 90%, 95%, 98%, 100% or in a range about between any two of these values. Such improvement or change may be observed in treated subjects as compared to subjects not treated with a F1C, where the untreated subjects have, or are subject to developing, the same or similar disease, condition, symptom or the like. Amelioration of a disease, condition, symptom or assay parameter may be determined subjectively or objectively, e.g., self assessment by a subject(s), by a clinician's assessment or by conducting an appropriate assay or measurement, including, e.g., a quality of life assessment, a slowed progression of a disease(s) or condition(s), a reduced severity of a disease(s) or condition(s), or a suitable assay(s) for the level or activity(ies) of a biomolecule(s), or by measuring one or more biological or clinical parameters. Amelioration may be transient, prolonged or permanent or it may be variable at relevant times during or after a F1C is administered to a subject or is used in an assay or other method described herein or a cited reference, e.g., within about 1 hour of the administration or use of a F1C to about 3, 6, 9 months or more after a subject(s) has received a F1C.

The “modulation” of, e.g., a biological parameter, symptom, level or biological activity of a molecule, cellular response, cellular activity or the like, means that the cell, level or activity, or the like is detectably increased or decreased. Such increase or decrease may be observed in treated subjects as compared to subjects not subjected to identical or similar biological insults. Such increases or decreases may be at least about 2%, 5%, 10%, 15%, 20%, 25%, 30%, 40%, 50%, 60%, 70%, 75%, 80%, 85%, 90%, 95%, 98%, 100%, 150%, 200%, 250%, 300%, 400%, 500%, 1000% or more or about within any range between any two of these values. Modulation may be determined subjectively or objectively, e.g., by the subject's self assessment, by a clinician's assessment or by conducting an appropriate assay or measurement, including, e.g., quality of life assessments or suitable assays for the level or activity of molecules, cells or cell migration within a subject. Modulation may be transient, prolonged or permanent or it may be variable at relevant times.

At various locations in the present disclosure, e.g., in any disclosed embodiments or in the claims, reference is made to compounds, compositions, formulations, or methods that “comprise” one or more specified components, elements or steps. Invention embodiments also specifically include those compounds, compositions, formulations or methods that are or that consist of or that consist essentially of those specified components, elements or method or process steps. The terms “comprising”, “consist of” and “consist essentially of” have their normally accepted meanings under U.S. patent law. For example, disclosed compositions or methods that “comprise” a component or step are open and they include or read on those compositions or methods plus an additional component(s) or step(s). Similarly, disclosed compositions or methods that “consist of” a component or step are closed and they would not include or read on those compositions or methods having appreciable amounts of an additional component(s) or an additional step(s).

“Alkyl” as used here, e.g., to describe F1Cs, means linked normal, secondary, tertiary or cyclic carbon atoms, i.e., linear, branched, cyclic or any combination thereof. Alkyl moieties, as used herein, may be saturated, or unsaturated, i.e., the moiety may comprise one, two, three or more independently selected double bonds or triple bonds. Unsaturated alkyl moieties include all moieties described for the alkenyl and alkynyl groups described below. The number of carbon atoms in an alkyl group or moiety can vary and typically is 1 to about 50, e.g., about 1-30 or about 1-20, unless otherwise specified, e.g., C₁₋₈ alkyl or C1-C8 alkyl means an alkyl moiety containing 1, 2, 3, 4, 5, 6, 7 or 8 carbon atoms. Alkyl groups will typically have 1, 2, 3, 4, 5, 6, 7, 8, 9, 10, 11, 12, 13, 14, 15, 16, 17, 18, 18 or 20 carbon atoms. When an alkyl group is specified, species may include methyl, ethyl, 1-propyl (n-propyl), 2-propyl (i-propyl, —CH(CH₃)₂), 1-butyl (n-butyl), 2-methyl-1-propyl (i-butyl, —CH₂CH(CH₃)₂), 2-butyl (s-butyl, —CH(CH₃)CH₂CH₃), 2-methyl-2-propyl (t-butyl, —C(CH₃)₃), 1-pentyl (n-pentyl), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl, 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl —C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃), cyclopropyl (—CH<CH₂CH₂), cyclobutyl (—CH<CH₂CH₂CH₂), cyclopentyl, cyclohexyl, cycloheptyl, cyclooctyl, —(CH₂)ₙ—(CHCH₃)ₘ—(CH₂)ₒ—CH₃, —(CH₂)ₙ—(CHC₂H₅)ₘ—(CH₂)ₒ—CH₃ and species and groups described below for alkenyl, alkynyl groups aryl groups, arylalkyl groups alkylaryl groups and the like, where n, m and o independently are 0, 1, 2, 3, 4, 5, 6, 7 or 8.

For any group or moiety described by a given range of carbon atoms, the designated range means that any individual number of carbon atoms is described. Thus, reference to, e.g., “C1-C4 optionally substituted alkyl”, “C₂₋₆ alkenyl”, or “C2-C6 optionally substituted alkenyl”, specifically means that a 1, 2, 3 or 4 carbon optionally substituted alkyl moiety as defined herein is present, or a 2, 3, 4, 5 or 6 carbon alkenyl or optionally substituted alkenyl moiety as defined herein is present. All such designations are expressly intended to disclose all of the individual carbon atom groups and thus “C1-C4 optionally substituted alkyl” means, e.g., 3 carbon alkyl, 4 carbon substituted alkyl and the like are disclosed and can be expressly referred to or named.

“Alkenyl” as used here means a moiety that comprises linked normal, secondary, tertiary or cyclic carbon atoms, i.e., linear, branched, cyclic or any combination thereof, that comprises one or more double bonds (e.g., —CH═CH—), e.g., 1, 2, 3, 4, 5, 6 or more, typically 1 or 2. The number of carbon atoms in an alkenyl group or moiety can vary and typically is 2 to about 50, e.g., about 2-30 or about 2-20, unless otherwise specified, e.g., C₂₋₈ alkenyl or C2-8 alkenyl means an alkenyl moiety containing 2, 3, 4, 5, 6, 7 or 8 carbon atoms. Alkenyl groups will typically have 2, 3, 4, 5, 6, 7, 8, 9, 10, 11, 12, 13, 14, 15, 16, 17, 18, 18 or 20 carbon atoms. When an alkenyl group is specified, species may include methylene (═CH₂), methylmethylene (═CH—CH₃), ethylmethylene (═CH—CH₂—CH₃), ═CH—CH₂—CH₂—CH₃, vinyl (—CH═CH₂), allyl, —(CH₂)ₙ—(CH═CH)—(CH₂)ₘ—CH₃, —(CH₂)ₙ—(CCH₃═CH)—(CH₂)ₘ—CH₃, —(CH₂)ₙ—(CH═CCH₃)—(CH₂)ₘ—CH₃ and —(CH₂)ₙ—(CH═CH)₀₋₁—(CH₂)ₘ—CH₂CH═CH₂, where n and m independently are 0, 1, 2, 3, 4, 5, 6, 7 or 8.

“Alkynyl” as used here means a moiety that comprises linked normal, secondary, tertiary or cyclic carbon atoms, i.e., linear, branched, cyclic or any combination thereof, that comprises one or more triple bonds (—C≡C—), e.g., 1, 2, 3, 4, 5, 6 or more, typically 1 or 2 triple bonds, optionally comprising 1, 2, 3, 4, 5, 6 or more double bonds, with the remaining bonds being single bonds. The number of carbon atoms in an alkenyl group or moiety can vary and typically is 2 to about 50, e.g., about 2-30 or about 2-20, unless otherwise specified, e.g., C₂₋₈ alkynyl or C2-8 alkynyl means an alkynyl moiety containing 2, 3, 4, 5, 6, 7 or 8 carbon atoms. Alkynyl groups will typically have 2, 3, 4, 5, 6, 7, 8, 9, 10, 11, 12, 13, 14, 15, 16, 17, 18, 18 or 20 carbon atoms. When an alkynyl group is specified, groups and species may include —CCH, —CCCH₃, —CCCH₂CH₃, —CCC₃H₇, —CCCH₂C₃H₇, —(CH₂)ₙ—(C≡C)—(CH₂)ₘ—CH₃, and —(CH₂)ₙ—(C≡C)₀₋₁(CH₂)ₘ—CH₂C≡CH, where n and m independently are 0, 1, 2, 3, 4, 5, 6, 7 or 8.

“Aryl” means an aromatic ring or fused ring system with no ring heteroatoms, e.g., phenyl or naphthyl.

“Arylalkyl” means a moiety where an alkyl group is bonded to an aryl group, i.e., -alkyl-aryl, where alkyl and aryl groups are as described above, e.g., —CH₂—C₆H₅ or —CH₂CH(CH₃)—C₆H₅.

“Alkylaryl” means a moiety where an aryl group is bonded to an alkyl group, i.e., -aryl-alkyl, where aryl and alkyl groups are as described above, e.g., —C₆H₄—CH₃ or —C₆H₄—CH₂CH(CH₃).

“Substituted alkyl”, “substituted alkenyl”, “substituted alkynyl”, “substituted alkylaryl”, “substituted arylalkyl”, “substituted heterocycle”, “substituted aryl”, “substituted monosaccharide” and the like mean an alkyl, alkenyl, alkynyl, alkylaryl, arylalkyl heterocycle, aryl, monosaccharide or other group or moiety as defined or disclosed herein that has a substituent(s) that replaces a hydrogen atom(s) or a substituent(s) that interrupts a carbon atom chain. Substituted heterocycles may thus have a substituent bonded to a ring carbon or a ring heteroatom such as a nitrogen. Any of these substituted groups will typically have 1, 2, 3, 4, 5, 6, 7, 8, 9, 10, 11, 12, 13, 14, 15, 16, 17, 18, 18 or 20 carbon atoms. Substituents for any of these moieties include 1, 2, 3, 4, 5, 6, 7, 8, 9, or 10 or more independently selected —O—, —S—, —NH—, —C(O)—, —C(O)OH, —C(O)OR^{15A}, —C(O)OR^{PR}, —C(O)SR^{15A}, —C(O)SR^{PR}, —CHO, —CHS, —CH₂SH, —C═N—, —OH, ═O, —OR^{15A}, —OR^{PR}, —C(O)OR^{PR}, —O—(O)H, —C(O)CH₃, —C(S)CH₃, —C(S)SH, —C(S)SR^{15A}, —C(S)SR^{PR}, —C(O)CH₂OH, —C(O)CH₂F, —C(O)CH₂Cl, —C(O)CH₂Br, —C(O)CH₂I, —C(O)CF₂H, —C(O)CF₃, —C(O)NHCH₃, —C(O)NHC₂H₅, —C(O)NHC(CH₃)₃, —O—CH₂—C(O)—C(CH₃)₃, —C(O)—C(CH₃)₃, —O—CH(CH₃)—O—C(CH₃)₃, —C(O)O—, —C(S)OR^{PR}, —C(S)O—, —OC(O)—, —C(O)H, —OCH₂—, —CH₂—O—CH₂—, —(CH₂)₁₋₂—O—(CH₂)₂, —OCH₂CH₂—, —OCH₂O—, —OCH₂CH₂O—, —CH₂OH, —CH₂F, —CHF₂, —CF₃, —CH₂Cl, —CH₂Br, —CH₂I, —C₂H₄Cl, —C₂H₄Br, —C₂H₄I, —CH₂CH₂F, —CH₂CHF₂, —CH₂CF₃, —NH₂, —NHR^{15A}, —N(R^{15A})₂, —N(R^{PR})₂, —NHR^{PR}, —NHC(O)—, —CH₂—NR^{PR}—, —CH₂—NHR^{PR}, —CH₂—NHC(O)—, —C(O)NH—, —C(O)NHR^{PR}, —OC(O)NR^{PR}, —OC(O)NHR^{PR}—C(═NH)—NH₂, —C(═NH)OH, —C(═N—NH₂)OH, —C(O)NHOH, ═NOH, ═NOCH₃, ═NOC₂H₅, ═NOC₃H₇, ═NOC₄H₉, —NHR^{15A}, ═NR^{15A}, ═N—, —NR^{PR}C(O)NR^{PR}—, —NR^{PR}C(O)NHR^{PR}, —NR^{PR}CH₂—, —NR^{PR}CH₂CH₂—, —NO₂, —ONO₂, —S—, —SH, —SR^{15A}, —SR^{PR}, ═S, —S(O)R^{15A}, —S(O)OR^{15A}, —S(O)—, —S(O)(O)—, —O—S(O)(O)—NR^{PR}—, —O—S(O)(O)—NR^{PR}—CH₂—, —CH₂—O—S(O)(O)—NR^{PR}—, —CHR^{15A}—S(O)(O)—NR^{PR}—, —CHR^{15A}—S(O)(O)—NR^{PR}—CHR^{15A}—, —NH—S(O)(O)H, —CH₂—NH—S(O)(O)H, —CHR^{15A}—NH—S(O)(O)H, —O—S(O)(O)—CHR^{15A}—, —CHR^{15A}—O—S(O)(O)—, —CHR^{15A}—O—S(O)(O)—CHR^{15A}—, —S—(O)(O)H, —CHR^{15A}—S(O)(O)H, —NH—S(O)(O)—NH—, —CHR^{15A}—NH—S(O)(O)—NH—, —CHR^{15A}—NH—S—(O)(O)—NH—CHR^{15A}, —NH—S(O)(O)—NHR^{PR}, —NH—S(O)(O)—NH₂, —NH—S(O)(O)—NHCH₃, —NH—S(O)—NH—, —CHR^{15A}—NH—S—(O)—NH—, —CHR^{15A}—NH—S(O)—NH—CHR^{15A}, —NH—S(O)—NHR^{PR}, —NH—S(O)—NH₂, —NH—S(O)—NHCH₃, —NH—S(O)—, —CHR^{15A}—NH—SO(O)—, —NH—S(O)—CHR^{15A}, —S(O)—NHR^{PR}, —S(O)—NH₂, —S(O)—NHCH₃, —S(O)(O)—O—, —S(O)OR^{PR}, —S(O)(O)OH, —OSO₃H₂, —S(O)(O)OR^{15A}, —S(O)(O)OR^{PR}, —S(O)OH, —S(O)OR^{15A}, —S(O)OR^{PR}, —S(O)R^{15A}, —S(O)R^{PR}, —CN, —SCN, —C(O)OH, —C(O)OR^{15A}, —C(O)OR^{PR}, —C(O)SH, —C(O)SR^{15A}, —C(O)SR^{PR}, —C(S)OH, —C(S)OR^{15A}, —C(S)OR^{PR}, —O—P(O)(O)OH, —O—P(O)(O)OR^{15A}, —O—P(O)(O)OR^{PR}, —O—P(S)(O)OH, —O—P(S)(O)OH, —O—P(S)(O)OR^{15A}, —O—P(S)(O)OR^{PR}, —O—P(O)(O)SH, —O—P(O)(O)SR^{15A}, —O—P(O)(O)SR^{PR}, —F, —Cl, —Br, —I, —C═NH, —C═NCH₃, —C═NC₂H₅, —C(═S)—, —C₆H₅, —CH₂C₆H₅, —O-A8, —S-A8,—C(O)-A8, —OC(O)-A8, —C(O)O-A8, —OPO₃(R^{PR})₂, -amino acid-, —O-monosaccharide, —O-disaccharide, —S-monosaccharide, —S-disaccharide, a polymer, e.g., a PEG, and combinations of these moieties and salts on any of these moieties that can form a salt, where each R^{PR} independently is —H, an independently selected protecting group or both R^{PR} together are a protecting group, A8 is C1-C10 optionally substituted alkyl, and R^{15A} independently are —H, —CH₃, —C₂H₅, —C₃H₇, —C₄H₉, —C(CH₃)₃, —CH₂OH, —C₂H₄OH, —C₃H₆OH, —C₄H₈OH—C(CH₂OH)(CH₃)₂, —C₃H₅, —C₄H₇, optionally substituted C1-10 alkyl, C1-10 perfluoroalkyl, optionally substituted aryl, optionally substituted C1-12 alkylaryl, optionally substituted C1-12 arylalkyl, optionally substituted allyl, optionally substituted heterocycle, optionally substituted C1-4 alkyl-optionally substituted heterocycle or optionally substituted heterocycle-optionally substituted C1-4 alkyl. Substituents are independently chosen when more than one is present. Alkenyl and alkynyl groups that comprise a substituent(s), are optionally substituted at a carbon that is one or more methylene moiety removed from the double bond, e.g., the substituent is optionally separated by one, two, three or more independently selected —CH₂—, —CH(C₁₋₆ optionally substituted alkyl)-, —CH(C₁₋₆ optionally substituted alkenyl)-, —CH(C₁₋₆ optionally substituted alkynyl)-, —CH(optionally substituted heterocycle)-, —CH(optionally substituted aryl-optionally substituted alkyl)- or —CH(optionally substituted alkyl-optionally substituted aryl)- moieties. Other substituted alkenyl and alkynyl moieties include ═CHOH, ═CH-halogen, ═CH—COOR^{PR}, ═CH—(CH₂)ₘ—NH₂, ═CH—(CH₂)ₘ—NH(C1-C6 alkyl), ═CH—N(C1-C6 alkyl)₂, ═CH—CH₂OH, ═CH—CH₂-halogen, ═CH—CH₂—COOR^{PR}, ═CH—CH₂—NH₂, ═CH—CH₂—NH(C1-C6 alkyl), ═CH—CH₂—N(C1-C6 alkyl)₂, ═CH—CH₂—CH₂OH, ═CH—CH₂—CH₂-halogen, ═CH—CHOH—CH₃, ═CH—CHOH—CH₂—CH₃, ═CH—CH₂—CH₂—COOR^{PR}, ═CH—CH₂—CH₂—NH₂, ═CH—CH₂—CH₂—N(C1-C4 alkyl)₂, —CH═CH—(CH₂)ₘ—OH, —CH═CH-halogen, —CH═CH—CH₂OH, —CH═CH—CH₂-halogen, —C≡C-halogen, —C≡C—CH₂—NH₂, —C≡C—CH₂—NH(C1-C6 alkyl), —C≡C—CH₂—N(C1-C6 alkyl)₂, —C≡C—OH, —C≡C—COOR^{PR}, —C≡C—CH₂-halogen, —C≡C—CH₂—OH and —C≡C—CH₂—COOR^{PR}, where each alkyl moiety is the same or different, e.g., both are methyl, ethyl or propyl or one is methyl and the other is ethyl, propyl or butyl and m is 1, 2, 3 or 4. The organic moieties and substitutions described here, and for other any other moieties described herein, usually will exclude obviously unstable moieties, e.g., —O—O—, except where such unstable moieties are transient species that one can use to make a compound such as a F1C with sufficient chemical stability for the one or more of the uses described herein.

“Heterocycle” or “heterocyclic” includes by way of example and not limitation the heterocycles described in Paquette, Leo A.; “Principles of Modern Heterocyclic Chemistry” (W. A. Benjamin, New York, 1968), particularly Chapters 1, 3, 4, 6, 7, and 9; “The Chemistry of Heterocyclic Compounds, A series of Monographs” (John Wiley & Sons, New York, 1950 to present), in particular Volumes 13, 14, 16, 19, and 28; and J. Am. Chem. Soc. 1960, 82:5566. Heterocycles are typically bonded to the steroid nucleus through a carbon, nitrogen or sulfur atom in the heterocycle ring.

Examples of heterocycles include by way of example and not limitation pyridyl, thiazolyl, tetrahydrothiophenyl, sulfur oxidized tetrahydrothiophenyl, pyrimidinyl, furanyl, thienyl, pyrrolyl, pyrazolyl, imidazolyl, tetrazolyl, benzofuranyl, thianaphthalenyl, indolyl, indolenyl, quinolinyl, isoquinolinyl, benzimidazolyl, piperidinyl, 4-piperidonyl, pyrrolidinyl, 2-pyrrolidonyl, pyrrolinyl, tetrahydrofuranyl, tetrahydroquinolinyl, tetrahydroisoquinolinyl, decahydroquinolinyl, octahydroisoquinolinyl, azocinyl, triazinyl, 6H-1,2,5-thiadiazinyl, 2H,6H-1,5,2-dithiazinyl, thienyl, thianthrenyl, pyranyl, isobenzofuranyl, chromenyl, xanthenyl, phenoxathiinyl, 2H-pyrrolyl, isothiazolyl, isoxazolyl, pyrazinyl, pyridazinyl, indolizinyl, isoindolyl, 3H-indolyl, 1H-indazoly, purinyl, 4H-quinolizinyl, phthalazinyl, naphthyridinyl, quinoxalinyl, quinazolinyl, cinnolinyl, pteridinyl, 4aH-carbazolyl, carbazolyl, β-carbolinyl, phenanthridinyl, acridinyl, pyrimidinyl, phenanthrolinyl, phenazinyl, phenothiazinyl, furazanyl, phenoxazinyl, isochromanyl, chromanyl, imidazolidinyl, imidazolinyl, pyrazolidinyl, pyrazolinyl, piperazinyl, indolinyl, isoindolinyl, quinuclidinyl, morpholinyl, oxazolidinyl, benzotriazolyl, benzisoxazolyl, oxindolyl, benzoxazolinyl, and isatinoyl.

By way of example and not limitation, carbon bonded heterocycles are bonded at position 2, 3, 4, 5, or 6 of a pyridine, position 3, 4, 5, or 6 of a pyridazine, position 2, 4, 5, or 6 of a pyrimidine, position 2, 3, 5, or 6 of a pyrazine, position 2, 3, 4, or 5 of a furan, tetrahydrofuran, thiofuran, thiophene, pyrrole or tetrahydropyrrole, position 2, 4, or 5 of an oxazole, imidazole or thiazole, position 3, 4, or 5 of an isoxazole, pyrazole, or isothiazole, position 2 or 3 of an aziridine, position 2, 3, or 4 of an azetidine, position 2, 3, 4, 5, 6, 7, or 8 of a quinoline or position 1, 3, 4, 5, 6, 7, or 8 of an isoquinoline. Still more typically, carbon bonded heterocycles include 2-pyridyl, 3-pyridyl, 4-pyridyl, 5-pyridyl, 6-pyridyl, 3-pyridazinyl, 4-pyridazinyl, 5-pyridazinyl, 6-pyridazinyl, 2-pyrimidinyl, 4-pyrimidinyl, 5-pyrimidinyl, 6-pyrimidinyl, 2-pyrazinyl, 3-pyrazinyl, 5-pyrazinyl, 6-pyrazinyl, 2-thiazolyl, 4-thiazolyl, or 5-thiazolyl.

By way of example and not limitation, nitrogen bonded heterocycles are bonded at position 1 of an aziridine, azetidine, pyrrole, pyrrolidine, 2-pyrroline, 3-pyrroline, imidazole, imidazolidine, 2-imidazoline, 3-imidazoline, pyrazole, pyrazoline, 2-pyrazoline, 3-pyrazoline, piperidine, piperazine, indole, indoline, 1H-indazole, position 2 of a isoindole, or isoindoline, position 4 of a morpholine, and position 9 of a carbazole, or β-carboline. Typically, nitrogen bonded heterocycles include 1-aziridyl, 1-azetedyl, 1-pyrrolyl, 1-imidazolyl, 1-pyrazolyl, and 1-piperidinyl.

“Heteroaryl” means an aromatic ring or two or more fused rings that contain one or more aromatic rings where the ring or fused rings comprise 1, 2, 3 or more heteroatoms, usually oxygen (—O—), nitrogen (—NX—) or sulfur (—S—) where X is —H, a protecting group or C₁₋₆ optionally substituted alkyl, usually —H. Examples are as described for heterocycle.

“Alcohol” as used herein means an alcohol that comprises a C₁₋₁₂ alkyl moiety substituted at a hydrogen atom with one hydroxyl group. Alcohols include methanol, ethanol, n-propanol, i-propanol, n-butanol, i-butanol, s-butanol, t-butanol, n-pentanol, i-pentanol, n-hexanol, cyclohexanol, n-heptanol, n-octanol, n-nonanol and n-decanol. The carbon atoms in alcohols can be straight, branched or cyclic. Alcohol includes any subset of the foregoing, e.g., C₁₋₄ alcohols (alcohols having 1, 2, 3 or 4 carbon atoms).

“Halogen” means fluorine, chlorine, bromine or iodine.

“Protecting group” means a moiety that prevents or reduces the atom or functional group to which it is linked from participating in unwanted reactions. For example, for —OR^{PR}, R^{PR} may be hydrogen or a protecting group for the oxygen atom found in a hydroxyl, while for —C(O)—OR^{PR}, R^{PR} may be hydrogen or a carboxyl protecting group, for —SR^{PR}, R^{PR} may be hydrogen or a protecting group for sulfur in thiols for instance, and for —NHR^{PR} or —N(R^{PR})₂—, R^{PR} may be hydrogen or a nitrogen atom protecting group for primary or secondary amines. Hydroxyl, amine, ketones and other reactive groups are found in F1Cs at, e.g., R¹ or R². These groups may require protection against reactions taking place elsewhere in the molecule. The protecting groups for oxygen, sulfur or nitrogen atoms are usually used to prevent unwanted reactions with electrophilic compounds, such as acylating agents used, e.g., in steroid chemistry.

“Ester” means a moiety that contains a —C(O)—O— structure. Typically, esters as used here comprise an organic moiety containing about 1-50 carbon atoms (e.g., about 2-20 carbon atoms) and 0 to about 10 independently selected heteroatoms (e.g., O, S, N, P, Si), where the organic moiety is bonded to a formula 1 steroid nucleus at, e.g., R¹ or R² through the —C(O)—O— structure, e.g., organic moiety-C(O)—O-steroid or organic moiety-O—C(O)-steroid. The organic moiety usually comprises one or more of any of the organic groups described herein, e.g., C₁₋₂₀ alkyl moieties, C₂₋₂₀ alkenyl moieties, C₂₋₂₀ alkynyl moieties, aryl moieties, C₂₋₉ heterocycles or substituted derivatives of any of these, e.g., comprising 1, 2, 3, 4 or more substituents, where each substituent is independently chosen. Exemplary substitutions for hydrogen or carbon atoms in these organic groups are as described above for substituted alkyl and other substituted moieties. Substitutions are independently chosen. The organic moiety includes compounds defined by the R₄ variable. The organic moieties exclude obviously unstable moieties, e.g., —O—O—, except where such unstable moieties are transient species that one can use to make a compound with sufficient chemical stability for one or more of the uses described herein, including for synthesis of the formula 1 or other compounds. The substitutions listed above are typically substituents that one can use to replace one or more carbon atoms, e.g., —O— or —C(O)—, or one or more hydrogen atom, e.g., halogen, —NH₂ or —OH. Exemplary esters include one or more independently selected acetate, enanthate, propionate, isopropionate, isobutyrate, butyrate, valerate, caproate, isocaproate, hexanoate, heptanoate, octanoate, nonanoate, decanoate, undecanoate, phenylacetate or benzoate, which are typically hydroxyl esters.

“Thioester” means a moiety that comprises a —C(O)—S— structure. Typically, thioesters as used here comprise an organic moiety containing about 1-50 carbon atoms (e.g., about 1-20 carbon atoms) and 0 to about 10 independently selected heteroatoms (e.g., O, S, N, P, Si), where the organic moiety is bonded to a formula 1 steroid nucleus at a variable group such as R¹, R², R³, R⁴ or R¹⁰ through the —C(O)—S— structure, e.g., organic moiety-C(O)—S-steroid or organic moiety-S—C(O)-steroid. The organic moiety is as described above for esters.

“Thionoester” means a moiety that comprises a —C(S)—O— structure. Typically, thionoesters as used here comprise an organic moiety containing about 1-50 carbon atoms (e.g., about 1-20 carbon atoms) and 0 to about 10 independently selected heteroatoms (e.g., O, S, N, P, Si), where the organic moiety is bonded to a formula 1 steroid nucleus at a variable group such as R¹, R², R³, R⁴ or R¹⁰ through the —C(S)—O— structure, e.g., organic moiety-C(S)—O-steroid or organic moiety-O—C(S)-steroid. The organic moiety is as described above for esters.

“Acetal”, “thioacetal”, “ketal”, “thioketal” “spiro ring” and the like mean a cyclic organic moiety that is bonded to a steroid ring atom in the F1Cs, e.g., steroid nucleus atoms at one, two or more of the 1, 2, 3, 4, 6, 7, 11, 12, 15, 16, 17, 18 or 19 positions. Typically, acetals comprise an organic moiety containing about 1-20 carbon atoms (e.g., about 1-10 carbon atoms) and 0 to about 10 independently selected heteroatoms (e.g., O, S, N, P, Si). For acetals (or ketals), the steroid nucleus atoms are usually carbons and the acetal is bonded to a steroid carbon through two oxygen atoms. Thioacetals (or thioketals) are bonded to the steroid nucleus through one oxygen and one sulfur atom or, more often, through two sulfur atoms. One, two or more of e.g., R¹, R², R³, R⁴, R¹⁰ at the 2, 11 or 15 positions, R^{10A}, R^{10B}, R^{10C} and R^{10D}, may be an independently selected acetal, thioacetal or spiro ring in any of the F1Cs disclosed herein. The oxygen or sulfur atoms in ketals and thioketals are linked by an optionally substituted alkyl moiety. Typically the alkyl moiety is an optionally substituted C1-C6 alkylene or branched alkyl structure such as —C(CH₃)₂—, —CH(CH₃)—, —CH₂—, —CH₂—CH₂—, —C[(C2-C4 alkyl)₂]_{1, 2, 3}- or —[CH(C2-C4 alkyl)]_{1, 2, 3}-. Acetals include moieties having the structure —O—[C(R³⁶)₂]₁₋₆—O—, —O—CH₂—[C(R³⁶)₂]₂—O—, —O—CH₂—CH₂—[C(R³⁶)₂]₂—O—, —O—CH₂—[C(R³⁶)₂]₂—CH₂—O—, and —O—CH₂—C(R³⁶)₂—O—, where each R³⁶ independently is —H, —OH, ═O, ═S, —SH, —F, —Cl, —Br, —I or an organic moiety such as C1-C6 alkyl (e.g., methyl, ethyl, hydroxymethyl or halomethyl), C2-C6 alkenyl, C2-C6 alkenyl, aryl or an heterocycle, any of which are optionally substituted, e.g., —CF₃ or —CH₂OH. In some of these embodiments, one R³⁶ is —H and the other is another atom or moiety, e.g., —OH, methyl or a halogen. In other embodiments, neither R³⁶ is —H, e.g., both are methyl. Thioacetals include moieties that comprise a —S—[C(R³⁶)₂]₁₋₆—O— or —S—[C(R³⁶)₂]₁₋₆—S— structure where the open valences are bonded to the same carbon on the steroid nucleus. Typically, thioacetals as used here comprise an organic moiety containing about 1-50 carbon atoms (e.g., about 2-20 carbon atoms) and 0 to about 10 independently selected heteroatoms (e.g., O, S, N, P, Si), where the organic moiety is bonded to a formula 1 steroid nucleus at variable groups such as R¹, R², R³, R⁴ or R¹⁰ through the —S—[C(R³⁶)₂]ₘ—O— or —S—[C(R³⁶)₂]ₘ—S— structure, e.g., 17-steroid-S—[C(R³⁶)₂]ₘ—O-17-steroid, 17-steroid-S—CH₂—CH₂—O-17-steroid, 17-steroid-O—[C(R³⁶)₂]ₘ—S-17-steroid, 17-steroid-S—[C(R³⁶)₂]ₘ—S-17-steroid, 17-steroid-S—[C(R³⁶)₂]ₘ—O-17-steroid, where m is 1, 2, 3, 4, 5 or 6. The organic moiety is as described above for esters. Other exemplary acetal and thioacetals are —O—C(CH₃)₂—O—, —O—CH₂—CH₂—CH₂—O—, —O—CH₂—CH₂—O—, —O—CH₂—O—, —O—C(CH₃)(heterocycle)-O—, —O—CH(heterocycle)-O—O—C(CH₃)(aryl)-O—, —O—CH(aryl)-O—, —S—C(CH₃)₂—O—, —S—C(CH₃)₂—S—, —S—CH₂-CH₂—O—, —S—CH₂—CH₂—S—, —S—CH₂—O—, —S—CH₂—S—, —O—C(CH₃)₂—CH₂—O—, —O—C(CH₃)₂—C(CH₃)₂—O—, —S—C(CH₃)₂—CH₂—O— and —O—C(CH₃)₂—CH₂—S—. Some of these moieties can serve as protecting groups for a ketone or hydroxyl, e.g., acetals such as —O—CH₂—CH₂—CH₂—O— or —O—CH₂—CH₂—O— for ketones, which form a spiro ring that can be removed by chemical synthesis methods or by metabolism in cells or biological fluids. For any spiro ring disclosed herein and unless otherwise specified, the 1^{st} and 2^{nd} open valences can be bonded to the carbon in the steroid nucleus in the α- and β-configurations respectively or in the α- and β-configurations respectively. For example, in a spiro —NH—CH₂—CH₂—O— structure, the 1^{st} open valence, i.e., at the nitrogen atom, can be, e.g., at the 17-position in the β-configuration and the 2^{nd} open valence, i.e., at the oxygen, would then be in the α-configuration.

“Phosphoester” or “phosphate ester” means a moiety that comprises a —O—P(OR^{PR})(O)—O— structure where R^{PR} is hydrogen (—H), a protecting group or an organic moiety as described for esters. Typically, phosphoesters as used here comprise a hydrogen atom, a protecting group or an organic moiety containing about 1-50 carbon atoms and 0 to about 10 independently selected heteroatoms (e.g., O, S, N, P, Si) linked to a formula 1 steroid nucleus at a variable group such as R¹-R⁶, R¹⁰, R¹⁵, R¹⁷ or R¹⁸ through the —O—P(O)(O)—O— structure, e.g., organic moiety-O—P(O)(OH)—O-steroid. The organic moiety is as described above for esters. Exemplary phosphoesters include —O—P(O)(OH)—O—CH₃, —O—P(O)(OCH₃)—O—CH₃, —O—P(O)(OH)—O—CH₂—CH₃, —O—P(O)(OC₂H₅)—O—CH₂—CH₃, —O—P(O)(OH)—O—CH₂—CH₂—CH₃, —O—P(O)(OH)—O—CH(CH₃)—CH₃)—CH₃, —O—P(O)(OH)—O—CH₂—CH₂—CH₂—CH₃, —O—P(O)(O(CH₃)₃)—O—C(CH₃)₃ and —O—P(O)(OH)—O—C(CH₃)₃.

“Phosphothioester” means a moiety that comprises a —O—P(SR^{PR})(O)—O— structure where R^{PR} is —H, a protecting group or an organic moiety as described for esters. Typically, phosphothioesters as used here comprise a hydrogen atom, a protecting group or an organic moiety containing about 1-50 carbon atoms and 0 to about 10 independently selected heteroatoms (e.g., O, S, N, P, Si) linked to a formula 1 steroid nucleus at a variable group such as R¹-R⁶, R¹⁰, R¹⁵, R¹⁷ or R¹⁸ through the —O—P(O)(O)—O— structure, e.g., organic moiety-O—P(O)(SH)—O-steroid. The organic moiety is as described above for esters. Exemplary phosphothioesters are as described for phosphoesters, except that sulfur replaces the appropriate oxygen atom.

“Phosphonoester” means a moiety that comprises a —P(OR^{PR})(O)— structure where R^{PR} is —H, a protecting group or an organic moiety as described for esters. Typically, phosphonoesters as used here comprise a hydrogen atom, a protecting group or an organic moiety containing about 1-50 carbon atoms and 0 to about 10 independently selected heteroatoms (e.g., O, S, N, P, Si) linked to a formula 1 steroid nucleus at a variable group such as R¹-R⁶, R¹⁰, R¹⁵, R¹⁷ or R¹⁸ through the —P(OR^{PR})(O)—O— structure, i.e., organic moiety-P(OR^{PR})(O)—O-steroid or steroid-P(OR^{PR})(O)—O-organic moiety. The organic moiety is as described above for esters.

“Phosphiniester” means a moiety that comprises a —P(O)H— structure where R^{PR} is —H, a protecting group or an organic moiety as described for esters. Typically, phosphiniesters as used here comprise a hydrogen atom, a protecting group or an organic moiety containing about 1-50 carbon atoms and 0 to about 10 independently selected heteroatoms (e.g., O, S, N, P, Si) linked to a formula 1 steroid nucleus at a variable group such as R¹-R⁶, R¹⁰, R¹⁵, R¹⁷ or R¹⁸ through the —P(O)H— structure, i.e., organic moiety-P(O)H-steroid or steroid-P(O)H-organic moiety. The organic moiety is as described above for esters.

“Sulfate ester” means a moiety that comprises a —O—S(O)(O)—O— structure. Typically, sulfate esters as used here comprise a hydrogen atom, a protecting group or an organic moiety containing about 1-50 carbon atoms and 0 to about 10 independently selected heteroatoms (e.g., O, S, N, P, Si) linked to a formula 1 steroid nucleus at a variable group such as R¹—R⁶, R¹⁰, R¹⁵, R¹⁷ or R¹⁸ through the —O—S(O)(O)—O— structure, e.g., organic moiety-O—S(O)(O)—O-steroid. The organic moiety is as described above for esters.

“Sulfite ester” means a moiety that comprises a —O—S(O)—O— structure. Typically, sulfite esters as used here comprise an organic moiety containing about 1-50 carbon atoms and 0 to about 10 independently selected heteroatoms (e g., O, S, N, P, Si) linked to a formula 1 steroid nucleus at a variable group such as R¹—R⁶, R¹⁰, R¹⁵, R¹⁷ or R¹⁸ through the —O—S(O)—O— structure, e.g., organic moiety-O—S(O)—O-steroid. The organic moiety is as described above for esters.

“Sulfamate ester”, “sulfamate derivative”, “sulfamate” and the like mean a moiety that comprises a —O—S(O)(O)—NH— or —O—S(O)(O)—NH₂ structure. Typically, sulfamate derivatives as used here comprise an organic moiety containing about 1-50 carbon atoms and 0 to about 10 independently selected heteroatoms (e.g., O, S, N, P, Si) linked to a formula 1 steroid nucleus at a variable group such as R¹—R⁶, R¹⁰, R¹⁵, R¹⁷ or R¹⁸ through a suitable structure such as —O—S(O)(O)—NH—, e.g., organic moiety-O—S(O)(O)—NH-steroid, steroid-O—S(O)(O)—NH-organic moiety or steroid-O—(O)(O)—NH₂. The organic moiety is as described above for esters.

“Sulfamide” and the like mean a moiety that comprises a —NH—S(O)(O)—NH— or —NH—S(O)(O)—NH₂ structure. Typically, sulfamide moieties comprise an organic moiety containing about 1-50 carbon atoms and 0 to about 10 independently selected heteroatoms (e.g., O, S, N, P, Si) linked to a formula 1 steroid nucleus at a variable group such as R¹—R⁶, R¹⁰, R¹⁵, R¹⁷ or R¹⁸ through a suitable structure such as —NH—S(O)(O)—NH—, e.g., steroid-NH—S(O)(O)—NH-organic moiety, steroid-NH—S(O)(O)—NH₂, steroid-NH—S(O)(O)—NHR^{PR} or steroid-NH—S(O)(O)—N(R^{PR})₂, where R^{PR} independently or together are a protecting group such as C1-C8 optionally substituted alkyl. The organic moiety is as described above for esters.

“Sulfinamide” and the like mean a moiety that comprises a —C—S(O)—NH-structure. Typically, sulfinamide moieties comprise an organic moiety containing about 1-50 carbon atoms and 0 to about 10 independently selected heteroatoms (e.g., O, S, N, P, Si) linked to a formula 1 steroid nucleus at a variable group such as R¹—R⁶, R¹⁰, R¹⁵, R¹⁷ or R¹⁸ through a suitable structure such as steroid-S(O)—NH-organic moiety, steroid-NH—S(O)-organic moiety, steroid-S(O)—NH₂, steroid-S(O)—NHR^{PR} moiety or steroid-S(O)—N(R^{PR})₂, where R^{PR} independently or together are a protecting group such as C1-C8 optionally substituted alkyl. The organic moiety is as described above for esters.

“Sulfurous diamide” and the like mean a moiety that comprises a —NH—S(O)—NH— or —NH—S(O)—NH₂ structure. Typically, sulfurous diamide moieties comprise an organic moiety containing about 1-50 carbon atoms and 0 to about 10 independently selected heteroatoms (e.g., O, S, N, P, Si) linked to a formula 1 steroid nucleus at a variable group such as R¹—R⁶, R¹⁰, R¹⁵, R¹⁷ or R¹⁸ through a suitable structure such as —C—NH—S(O)—NH—C— or —CH₂—NH—S(O)—NH—CH₂—, e.g., steroid-NH—S(O)—NH-organic moiety, steroid-NH—S(O)—NH₂, steroid-NH—S(O)—NHR^{PR} or steroid-NH—S(O)—N(R^{PR})₂, where R^{PR} independently or together are a protecting group such as C1-C8 optionally substituted alkyl. The organic moiety is as described above for esters.

“Sulfonate ester”, “sulfonate derivative”, “sulfonate” and the like mean a moiety that comprises a —O—S(O)(O)— or —S(O)(O)—O— structure. Typically, sulfonate derivatives comprise an organic moiety containing about 1-50 carbon atoms and 0 to about 10 independently selected heteroatoms (e.g., O, S, N, P, Si) linked to a formula 1 steroid nucleus at a variable group such as R¹—R⁶, R¹⁰, R¹⁵, R¹⁷ or R¹⁸ through a suitable structure such as —S(O)(O)—O—, e.g., organic moiety-O—S(O)(O)-steroid, HO—S(O)(O)-steroid or organic moiety-S(O)(O)—O-steroid. The organic moiety is as described above for esters.

“Amide”, “amide derivative” and the like mean an organic moiety as described for ester that comprises a —C(O)—NR^{PR}— or —C(O)—NH— moiety, where R^{PR} is —H or a protecting group. In some embodiments, the —C(O)NR^{PR}— group is linked to the steroid nucleus at a variable group such as R¹-R⁶, R¹⁰, R¹⁵, R¹⁷ or R¹⁸, i.e., organic moiety organic moiety-C(O)—NH-steroid or steroid-C(O)NR^{PR}-organic moiety. The organic moiety is as described above for esters.

“Ether” means an organic moiety as described for ester that comprises 1, 2, 3, 4 or more —O— moieties, usually 1 or 2. In some embodiments, the —O— group is linked to the steroid nucleus at a variable group such as R¹—R⁶, R¹⁰, R¹⁵, R¹⁷ or R¹⁸, e.g., organic moiety-O-steroid. The organic moiety is as described above for esters.

“Thioether” means an organic moiety as described for ester that comprises 1, 2, 3, 4 or more —S— moieties, usually 1 or 2. In some embodiments, the —S— group is linked to the steroid nucleus at a variable group such as R¹—R⁶, R¹⁰, R¹⁵, R¹⁷ or R¹⁸, e.g., organic moiety-S-steroid, organic moiety-S—CH₂—S-steroid or organic moiety-S—S-steroid. The organic moiety is as described above for esters.

“Acyl group” or “acyl” means an organic moiety as described for ester that comprises 1, 2, 3, 4 or more —C(O)— groups. In some embodiments, the —C(O)— group is linked to the steroid nucleus at a variable group such as R¹—R⁶, R¹⁰, R¹⁵, R¹⁷ or R¹⁸, e.g., organic moiety-C(O)-steroid. The organic moiety is as described above for esters. Exemplary acyl moieties include moieties such as —C(O)—N(C1-C6 alkyl)₂, —C(O)—NH(C1-C6 alkyl), —C(O)—NH—C(CH₃)₃, —C(O)—NH—CH(CH₃)₂, —C(O)—NH—C(CH₃)₂—CH₃, —C(O)—NH—CH(CH₃)—CH₃, —C(O)—NH—C(CH₃)—CH₂—CH₃, —C(O)NH₂, —C(O)NHR^{PR}, —C(O)—CH₃, —C(O)—CH₂—CH₃, —C(O)—CH₂—CH₂—CH₃, —C(O)—CH₂OH, —C(O)—CH₂OR^{PR}, —C(O)—CH₂—CH₂OH, —C(O)—CH₂—CH₂OR^{PR}, —C(O)—CH₂-halogen, —C(O)—CH₂—CH₂-halogen, —C(O)—CH₂—COOR^{PR}, —C(O)—CH₂—CH₂—COOR^{PR}, —C(O)—CH₂—CH₂—CHOH, —C(O)—CH₂—NH₂, —C(O)—CH₂—NHR^{PR}, —C(O)—CH₂—N(R^{PR})₂, —C(O)—CH₂—NH—(C1-C6 alkyl), —C(O)—CH₂—N(C1-C6 alkyl)₂, —C(O)—NH—CH═CH₂, —C(O)—NH—C═CH, —C(O)—NH—CH₃, —C(O)—NH—CN, —C(O)—NH—CH₂—CN, where each alkyl is the same or different and is optionally independently substituted and each R^{PR} is —H or an independently selected protecting group for the atom or functional group to which it is attached, or two R^{PR} together are a protecting group for the atom or functional group to which they are attached.

“Thioacyl” means an organic moiety as described for ester that comprises 1, 2, 3, 4 or more —C(S)— groups. In some embodiments, the —C(S)— group is linked to the steroid nucleus at a variable group such as R¹—R⁶, R¹⁰, R¹⁵, R¹⁷ or R¹⁸, e.g., organic moiety-C(S)-steroid. The organic moiety is as described above for esters. Exemplary thioacyl moieties include moieties as described above for the acyl group, except that sulfur replaces the appropriate oxygen atom.

“Carbonate” means an organic moiety as described for ester that comprises 1, 2, 3, 4 or more —O—C(O)—O— structures. Typically, carbonate groups as used here comprise an organic moiety containing about 1-50 carbon atoms and 0 to about 10 independently selected heteroatoms (e.g., O, S, N, P, Si) linked to a formula 1 steroid nucleus at a variable group such as R¹—R⁶, R¹⁰, R¹⁵, R¹⁷ or R¹⁸ through the —O—C(O)—O— structure, e.g., organic moiety-O—C(O)—O-steroid. The organic moiety is as described above for esters.

“Carbamate” means an organic moiety as described for ester that comprises 1, 2, 3, 4 or more —O—C(O)NR^{PR}— structures where R^{PR} is —H, a protecting group or an organic moiety as described for ester. Typically, carbamate groups as used here comprise an organic moiety containing about 1-50 carbon atoms and 0 to about 10 independently selected heteroatoms (e.g., O, S, N, P, Si) linked to a formula 1 steroid nucleus at a variable group such as R¹—R⁶, R¹⁰, R¹⁵, R¹⁷ or R¹⁸ through the —O—C(O)—NR^{PR}— structure, e.g., organic moiety-O—C(O)—NR PRsteroid or steroid-O—C(O)—NR^{PR}-organic moiety. The organic moiety is as described above for esters.

As used herein, “monosaccharide” means a polyhydroxy aldehyde or ketone having the empirical formula (CH₂O)ₙ where n is 3, 4, 5, 6 or 7. Monosaccharide includes open chain and closed chain forms, but will usually be closed chain forms. Monosaccharide includes hexofuranose and pentofuranose sugars such as 2′-deoxyribose, ribose, arabinose, xylose, their 2′-deoxy and 3′-deoxy derivatives and their 2′,3′-dideoxy derivatives. Monosaccharide also includes the 2′,3′ dideoxydidehydro derivative of ribose. Monosaccharides include the D-, L- and DL-isomers of glucose, fructose, mannose, idose, galactose, allose, gulose, altrose, talose, fucose, erythrose, threose, lyxose, erythrulose, ribulose, xylulose, ribose, arabinose, xylose, psicose, sorbose, tagatose, glyceraldehyde, dihydroxyacetone and their monodeoxy or other derivatives such as rhamnose and glucuronic acid or a salt of glucuronic acid. Monosaccharides are optionally protected or partially protected. Exemplary monosaccharides include

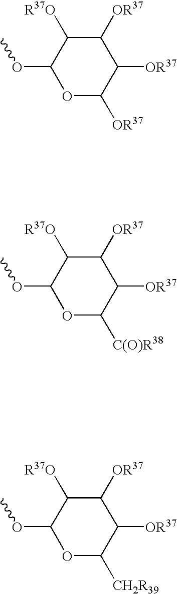

where R³⁷ independently is hydrogen, a protecting group, acetamido (—NH—Ac), optionally substituted alkyl such as methyl or ethyl, or an ester such as acetate or proprionate, R³⁸ is hydrogen, hydroxyl, —NH₂, —NHR^{PR}, optionally substituted alkyl such as methyl or ethyl, or a cation such as NH₄⁺, Na⁺ or K⁺ and R³⁹ is hydrogen, hydroxyl, acetate, proprionate, optionally substituted alkyl such as methyl, ethyl, methoxy or ethoxy.

Monosaccharides and disaccharides are optionally bonded at one or more of R¹, R⁴ or other variable groups in any F1C include 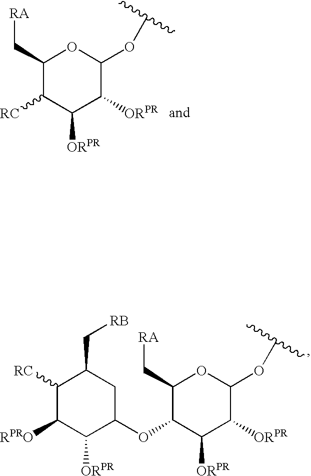
where RA and RB independently are —H, —OH, halogen, —NH₂, —NHR^{PR}, —N₃, C1-C6 alkoxy or —RD-RE, RC is —H, —OH, halogen, —NH₂, —NHR^{PR}, —N₃, C1-C6 alkoxy or a monosaccharide or disaccharide linked through a glycosidic bond, RD is —NH—C(O)—, —O—C(O)—, —O—C(O)—N(R^{PR})—, —NH—C(O)—N(R^{PR})—, —O—C(S)—N(R^{PR})— or —O—C(O)—N—(R^{PR})—, RE is aryl, arylalkyl, alkenyl, alkyl, cycloalkyl or cycloalkyl-alkyl, where each RE is optionally independently substituted with 1, 2 or 3 independently selected halogens, —OH, ═O, —SH, ═S, —NO₂, —CF₃, C1-C6 alkyl, phenoxy, C1-C6 alkoxy, methylenedioxy, C1-C6 alkylsulfanyl, C1-C6 alkylsulfinyl, C1-C6 alkylsulfonyl, dimethylamino, mono- or di-C1-C6 alkylaminocarbonyl, C1-C6 alkylcarbonyl, C1-C6 alkoxycarbonyl or pyrrolidinylcarbonyl, R^{PR} independently is —H or a protecting group such as C1-C6 optionally substituted alkyl, ester such as acetate or, if bonded to nitrogen, R^{PR} together with the nitrogen to which it is attached is pyrrolidinyl, piperidinyl, N-methylpiperazinyl, indolinyl or morpholinyl, where the cyclic group may be monosubstituted on a carbon atom with C1-C6 alkoxycarbonyl or C1-C6 optionally substituted alkyl. In some of these embodiments, RA, RB and RC are —OH.

Optionally substituted alkyl group, optionally substituted alkenyl group, optionally substituted alkynyl group, optionally substituted aryl moiety and optionally substituted heterocycle mean an alkyl, alkenyl, alkynyl, aryl or heterocycle moiety that contains an optional substitution(s). Such moieties include C₁₋₂₀ alkyl moieties, C₂₋₂₀ alkenyl moieties, C₂₋₂₀ alkynyl moieties, aryl moieties, C₂₋₉ heterocycles or substituted derivatives of any of these.

Optionally substituted “monosaccharide” comprise any C3-C7 sugar, D-, L- or DL-configurations, e.g., erythrose, glycerol, ribose, deoxyribose, arabinose, glucose, mannose, galactose, fucose, mannose, glucosamine, N-acetylneuraminic acid, N-acetylglucosamine, N-acetylgalactosamine that is optionally substituted at one or more hydroxyl groups or hydrogen or carbon atoms. Suitable substitutions are as described above for substituted alkyl moieties and include independently selected hydrogen, hydroxyl, protected hydroxyl, carboxyl, azido, cyano, —O—C₁₋₆ alkyl, —S—C₁₋₆ alkyl, —O—C₂₋₆ alkenyl, —S—C₂₋₆ alkenyl, ester, e.g., acetate or proprionate, optionally protected amine, optionally protected carboxyl, halogen, thiol or protected thiol. The linkage between the monosaccharide and the steroid is α or β.

Optionally substituted “oligosaccharide” comprises two, three, four or more of any C3-C7 sugars that are covalently linked to each other. The linked sugars may have D-, L- or DL-configurations. Suitable sugars and substitutions are as described for monosaccharides. The linkage between the oligosaccharide and the steroid is α or β, as are the linkages between the monosaccharides that comprise the oligosaccharide. Adjacent monosaccharides may be linked by, e.g., 1→2, 1→3, 1→4, and/or 1→6 glycosidic bonds.

Nucleoside includes 3TC, AZT, D4T, ddl, ddC, G, A, U, C, T, dG, dA, dT and dC.

Polymer includes biocompatible organic polymers, e.g., polyethyleneglycols (“PEGs”) and polyhydroxyalkyl polymers. PEG means an ethylene glycol polymer that contains about 2, 3, 4, 5, 6, 7, 8, 9, 10, 11, 12 or more linked monomers, e.g., about 50-1000 linked monomers. Average molecular weights typically are about 80, 100, 200, 300, 400 or 500, and mixtures thereof may are included, e.g., PEG100 and PEG200, PEG200 and PEG300, PEG100 and PEG300 or PEG200 and PEG400.

Embodiments of F1Cs. The F1Cs that can be used in the treatment and characterization methods described herein have the structure

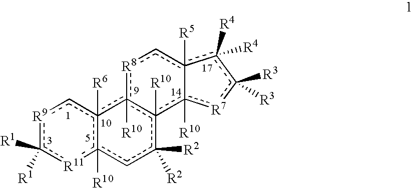

or a metabolic precursor, a metabolite, salt or tautomer thereof, wherein the dotted lines are optional double bonds and 0, 1, 2, 3, 4, 5 or more double bonds are present, some of which may be conjugated, each R¹, R², R³, R⁴, R⁵, R⁶and R¹⁰ independently or together are —H, —OH, —OR^{PR}, —SR^{PR}, —SH, —N(R^{PR})₂, —NHR^{PR}, —NH₂, —O—Si—(R¹³)₃, —CHO, —CHS, —CN, —SCN, —NO₂, —N₃, —COOH, —COOR^{PR}, —OSO₃H, —OSO₂H, —OPO₃H₂, ═O, ═S, ═N—OH, ═N—OCH₃, ═CH₂, ═CH—CH₃, ═CH-optionally substituted alkyl, ester, thioester, thionoester, phosphoester, phosphothioester, phosphonate, phosphonate ester, thiophosphonate, thiophosphonate ester, phosphiniester, sulfite ester, sulfate ester, sulfamate, sulfonate, sulfonamide, amide, amino acid, peptide, ether, thioether, acyl, thioacyl, carbonate, carbamate, halogen, optionally substituted alkyl, optionally substituted alkenyl, optionally substituted alkynyl, optionally substituted aryl, optionally substituted heteroaryl, optionally substituted heterocycle, optionally substituted monosaccharide, optionally substituted oligosaccharide, polymer, spiro ring, epoxide, acetal, thioacetal, ketal, thioketal, —S—S-optionally substituted alkyl, ═N—O-optionally substituted alkyl, ═N-optionally substituted alkyl, —NH-optionally substituted alkyl, —NH—S(O)(O)-optionally substituted alkyl, —N(optionally substituted alkyl)₂ where each optionally substituted alkyl is independently selected, or, one or more of two adjacent R¹, R², R³, R⁴, R⁵, R⁶ and R¹⁰ comprise an independently selected epoxide or optionally substituted saturated or unsaturated cyclopropyl, cyclobutyl, cyclopentyl, cyclohexyl, cycloheptyl or cyclooxyl ring any of which rings optionally contain a ring heteroatom such as —O—, —S—, —NH— or ═N—; R⁷ is —O—, —S—, —S(O)(O)—, —NR^{PR}—, —C(R¹⁰)₂—, —C(R¹⁰)₂—C(R¹⁰)₂—, —C(R¹⁰)₂—C(R¹⁰)₂—C(R¹⁰)₂—, —C(R¹⁰)₂—O—C(R¹⁰)₂—, —C(R¹⁰)₂—NR^{PR}—C(R¹⁰)₂—, —O—C(R¹⁰)₂—, —S—C(R¹⁰)₂— or —NR^{PR}—C(R¹⁰)₂—, where each R¹⁰ is independently selected; R⁸ and R⁹ independently are —C(R¹⁰)₂—, —C(R¹⁰)₂—C(R¹⁰)₂—, —O—, —O—C(R¹⁰)₂—, —S—, —S(O)(O)—, —S—C(R¹⁰)₂—, —S(O)(O)—C(R¹⁰)₂—, —NR^{PR}— or —NR^{PR}—C(R¹⁰)₂—, or one or both of R⁸ or R⁹ independently are absent, leaving a 5-membered ring, where each R¹⁰ is independently selected; R¹¹ is —O—, —S—, —S(O)(O)—, —NR^{PR}—, —CH₂—, —CHR¹⁰—, —C(R¹⁰)₂—, —C(R¹⁰)₂—O—C(R¹⁰)₂—, —C(R¹⁰)₂—S—C(R¹⁰)₂—, —C(R¹⁰)₂—NR^{PR}—C(R¹⁰)₂—, —O—C(R¹⁰)₂—, —S—C(R¹⁰)₂—, —S(O)(O)—C(R¹⁰)₂— or —NR^{PR}—C(R¹⁰)₂—, where each R¹⁰ is independently selected; R¹³ independently is C₁₋₆ alkyl; R^{PR} independently are —H or a protecting group; and optionally wherein one, two or three of the 1-, 4-, 6- and/or 12-positions are optionally substituted with (i) an independently selected R¹⁰ moiety when a double bond is present at the corresponding 1-, 4-, 6- or 12-position, or (ii) one or two independently selected R¹⁰ moieties when no double bond is present at the corresponding 1-, 4-, 6- and/or 12-position.

When two variable groups are present, ,e.g., two R¹, R³, R⁴ or R¹⁰, the groups can be the same or different. As is apparent from the F1C description, both variable groups at a given position cn both be —H. However, when a variable group is not —H, one variable group can be —H or a C-linked moiety, while the other variable Igroup may be an O-linked moiety, S-linked moiety, or N-linked moiety. In other cases, one variable group is a C-linked moiety, while the other variable Igroup is a C-linked moiety, O-linked moiety, S-linked moiety, or N-linked moiety. Both variable groups at a given position can be a C-linked moiety, O-linked moiety, S-linked moiety, or N-linked moiety, where each moiety is the same, different or where they form a ring such as a spiro ring.

A ‘C-linked moiety’ or ‘C-bonded moiety’ is a substituent that is bonded to the steroid through a carbon atom, e.g., an optionally substituted alkyl group such as —CH₃ or —C₂H₅. An ‘O-linked moiety’ or ‘O-bonded moiety’ is a substituent that is bonded to the steroid through an oxygen atom, e.g., an ether or ester. Similarly, an ‘S-linked moiety’ or ‘S-bonded moiety’ is a substituent that is bonded to the steroid through a sulfur atom atom, e.g., a thioether, and an ‘N-linked moiety’ or ‘N-bonded moiety’ is a substituent that is bonded to the steroid through a nitrogen atom, e.g., an amide or carbamate such as —NH—C(O)—CH₃ or —NH—C(O)—O—CH₃.

Invention embodiments include (1) compositions that comprise a formula 1 compound and one or more other compounds such as an excipient(s) or a reactant or by-product of synthesis of the formula 1 compound, (2) formulations that comprise a formula 1 compound and 1, 2, 3, 4, 5, 6 or more excipients and (3) compositions that comprise partially purified or purified formula 1 compounds, optionally in a composition that comprises 1, 2, 3, 4, 5, 6 or more excipients and/or other compounds. The formulations can be designed for human or pharmaceutical use or they can be suitable for veterinary use. A purified F1 C will usually be at least about 80% w/w pure or at least about 90% w/w pure or at least about 95% w/w or at least about 97% pure w/w, while partially purified F1Cs are typically at least about 30% w/w pure or at least about 94% w/w pure or at least about 50% w/w or at least about 60% w/w pure.

Therapeutic uses include the use of a formula 1 compound for the preparation of a medicament and use of a formula 1 compound for the preparation of a medicament for the prophylaxis, treatment or amelioration of a condition or symptom disclosed herein. Other embodiments are as described elsewhere in the specification or the claims.

As used herein, position numbers that are given for the F1Cs use the numbering convention for cholesterol. When a variable group such as R⁸ or R⁹ is absent and the ring is contracted to a 5-membered ring, the numbering of remaining ring atoms is not changed. Thus, when R⁹ is absent, ring numbering is as shown below.

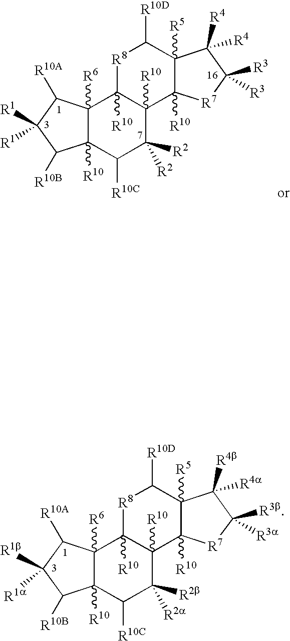

As shown in these structures, when two variable groups such as R¹, R², R³ or R⁴ are shown they may be in the α- or β-configuration and this may be specified in the variable group or it may not be specified, e.g., R^{1α} is an R¹ group in the α-configuration and R^{4β} is an R⁴ group in the β-configuration. R^{10A}, R^{10B}, R^{10C} and R^{10D} are independently chosen R¹⁰ moieties, where each is in the α- or β-configuration when no double bond is present in the steroid ring to which the R^{10A}, R^{10B}, R^{10C} and R^{10D} moiety is bonded. When R⁸ is absent, ring numbering is as shown

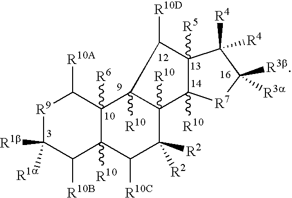

As is apparent from the foregoing, variable groups may or may not be specified in chemical structures as being in the α- or β-configuration. For any of these structures, 1, 2, 3, 4, 5 or more double bonds may be present at any of the steroid ring positions, and if double bonds are present, one variable group at the each position of the double may be absent. Double bonds in the steroid rings may thus be present at the 1-, 2-, 3-, 4-, 5-, 5(10)-, 6-, 7-, 8-, 9-, 9(11)-, 11-, 12-, 13(17)-, 14-, 15- and/or 16-positions for any of these structures or any othe F1C structures shown herein. Exemplary structures where R⁹ is absent and a double bond is present that displaces a variable group can have the structure

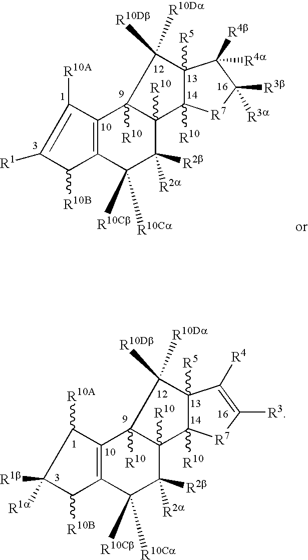

Spiro ring substituents are cyclic structures that are usually 3, 4, 5, 6, 7 or 8 membered rings, e.g., they include 3-, 4-, 5-, 6-, 7- or 8-sided rings. In some embodiments, spiro structures share a carbon atom that is present in the steroid ring system, e.g., at the 2, 3, 7, 11, 15, 16 or 17 positions of the F1Cs. Spiro structures include, acetals, thioacetals and lactone rings or cyclic esters. Spirolactones, spiro ring compounds and dihydroxy F1Cs containing cyclic diol groups include F1Cs having the structures

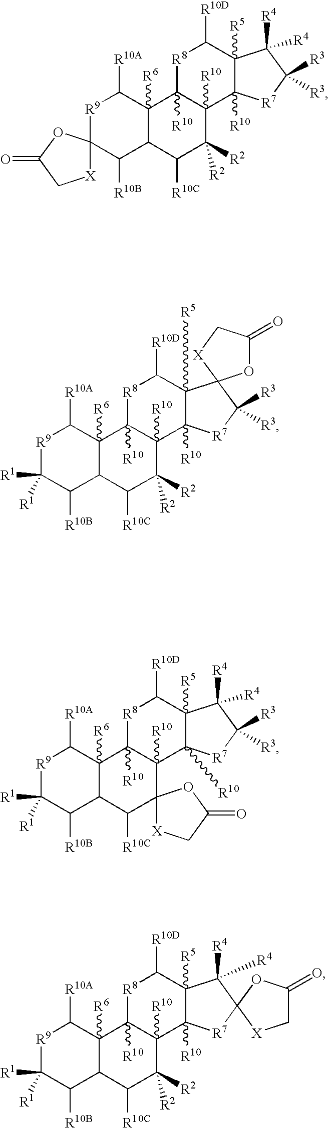

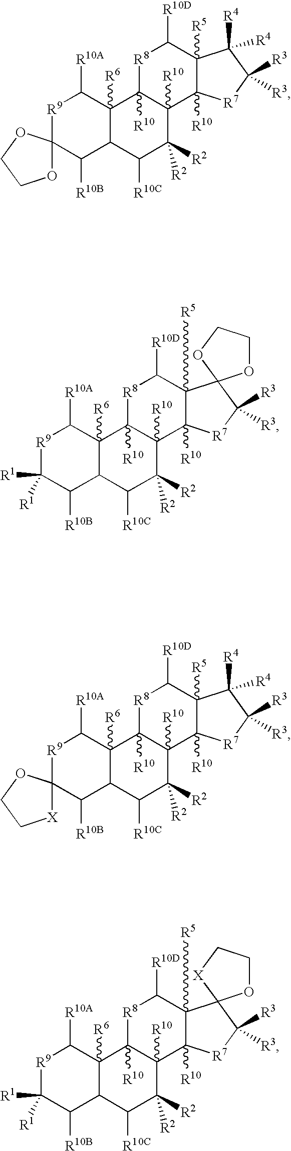

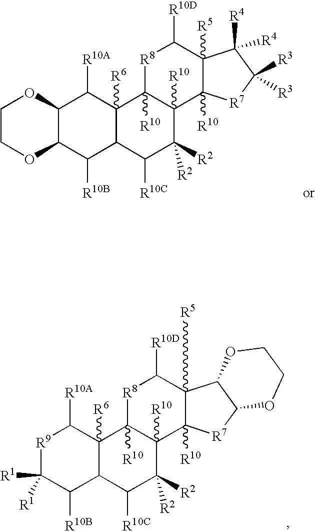

where 0, 1, 2, 3, 4 or 5 double bonds are present in the steroid rings, X is —C(R¹⁰)₂— or —CHR¹⁰—, and R¹⁰ are independently selected. In some of these embodiments, the R¹⁰, R^{10A}, R^{10B}, R^{10C} and R^{10D} variable groups are in the α- or β-configuration and are independently selected from —H, —F, —Cl, —Br, —OH, —OCH₃, —OC₂H₅, an optionally substituted ester such as acetate or propionate, an optionally substituted alkyl such as methyl or ethyl or an amino acid.

F1C structures 5, 6, 7, 8, 9, 10, 11, 12, 13 or 14 are

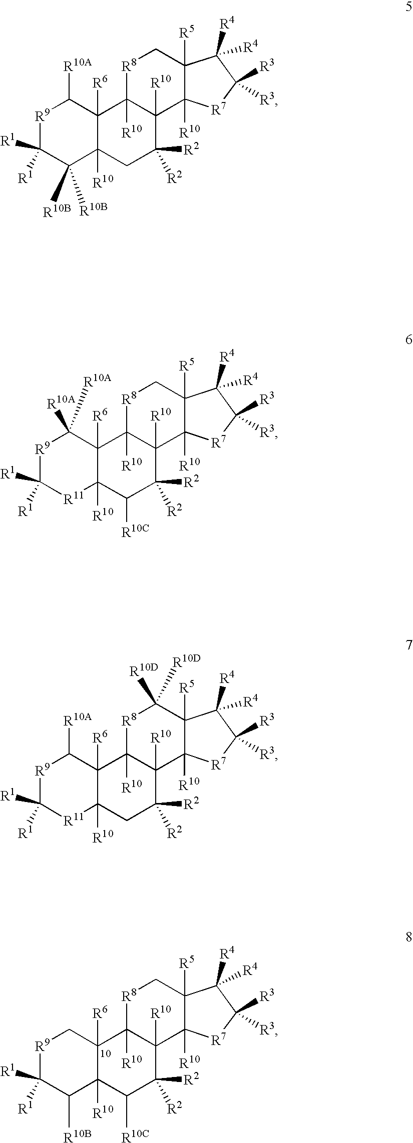

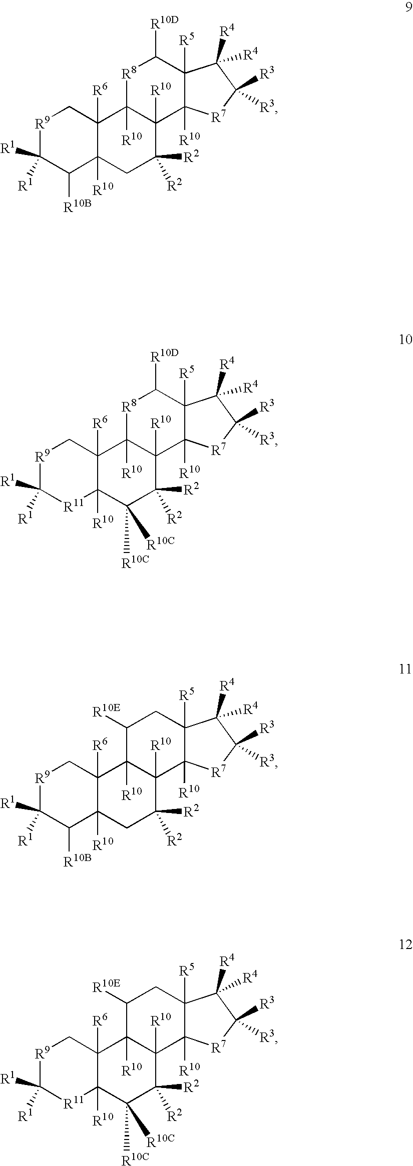

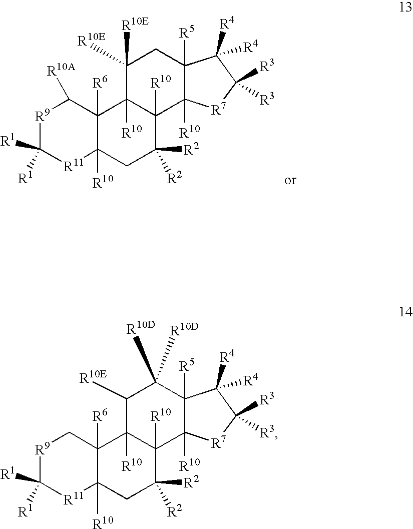

or a metabolic precursor or a metabolite thereof, wherein 0, 1, 2, 3, 4, 5 or more double bonds are present in the steroid rings, R¹⁰ moieties at the 5 (if present), 8, 9 and 14 positions respectively are in the α,α,αα, α,α,α,β, α,β,α,α, β,α,α,α, α,α,β,β, α,β,α,β, β,α,α,β, β,α,β,α, β,β,α,α, α,β,β,α, α,β,β,β, β,α,β,β, β,β,α,ββ,β,β,α, β,β,β,α or β,β,β,βconfigurations, R^{10A}, R^{10B}, R^{10C}, R^{10D} and R^{10E} respectively are in the α,α, α,β, β,α or β,β configurations and they are independently selected R¹⁰ moieties, or when two R^{10A}, R^{10B}, R^{10C}, R^{10D} or R^{10E} are present, each is an independently selected R¹⁰ moiety. Other variable groups are as elsewhere defined.

For formula 1 compounds (“F1Cs”), 2, 3 or more of R¹, R², R³ and R⁴ are usually not —H, and typically one or both R¹ and R⁴, R³ and R⁴, R², R³ and R⁴ or R² and R⁴ are not —H, and/or 1 or 2 of R^{10A}, R^{10B}, R^{10C} and R^{10D} are optionally not —H. For any F1C disclosed herein, steroid nucleus carbon atoms that contain two variable groups (e.g., two R¹⁰ at R⁸ or R⁹ or two R³ or R⁴ at the 16- or 17-position), each variable group is independently selected and each can thus be the same or different, e.g., both can be methyl, ethyl, methoxy, ethoxy, —F, —Cl, —Br, —I, or they can be different. As is apparent from the F1C structures, a double bond can be present at either the 4-5 position or at the 5-6 position, but not at both positions at the same time. Steroid nucleus carbon atoms refers generally to the carbons that make up the rings in F1Cs and carbons, if present, that are bonded to the 10, 13 and 17 positions. Additional carbons that may be at the 17-position are typically numbered using the cholesterol numbering system, although any other suitable nomenclature can be used to describe species or genera of F1C. Exemplary F1C embodiments are described below.

F1Cs include 16α-bromoepiandrosterone hemihydrate which has previously been described, e.g., WO 00/56757. This compound is used as a F1C either as a pure compound or substantially free of other forms, such as amorphous or anhydrous forms.

Salts and complexes of F1Cs, including pharmaceutically acceptable or salts that are relatively non-toxic, can be incorporated into treatment protocols. Some of the F1Cs have one or more moieties that carry at least a partial positive or negative charge in aqueous solutions, typically at a pH of about 4-10, that can participate in forming a salt, a complex, a composition with partial salt and partial complex properties or other noncovalent interactions, all of which we refer to as a “salt(s)”. Salts are usually biologically compatible or pharmaceutically acceptabje or non-toxic, particularly for mammalian cells. Salts that are biologically toxic are optionally used with synthetic intermediates of F1Cs. When a water-soluble composition is desired, monovalent salts are usually used.

Salt(s) of F1Cs may comprise a combination of appropriate cations such as alkali and alkaline earth metal ions or ammonium and quaternary ammonium ions with the acid anion moiety of the phosphoric acid or phosphonic acid group, which may be present in polymers or monomers. Metal salts include ones containing Na⁺, Li⁺, K⁺, Ca⁺⁺ or Mg⁺⁺. Other metal salts may contain aluminum, barium, strontium, cadmium, bismuth, arsenic or zinc ion.

Suitable amine salts include amines having sufficient basicity to form a stable salt, usually amines of low toxicity including trialkyl amines (tripropylamine, triethylamine, trimethylamine), procaine, dibenzylamine, N-benzyl-betaphenethylamine, ephenamine, N,N′-dibenzylethylenediamine, N-ethylpiperidine, benzylamine and dicyclohexylamine.

Salts include organic sulfonic acid or organic carboxylic acid salts, made for example by addition of the acids to basic centers, typically amines. Exemplary sulfonic acids include C₆₋₁₆ aryl sulfonic acids, C₆₋₁₆ heteroaryl sulfonic acids and C₄₋₁₆ alkyl sulfonic acids such as phenyl sulfonic acid, a-naphthalene sulfonic acid, β-naphthalene sulfonic acid, (S)-camphorsulfonic acid, methyl (CH₃SO₃H), ethyl (C₂H₅SO₃H), n-propyl, i-propyl, n-butyl, s-butyl, i-butyl, t-butyl, pentyl and hexyl sulfonic acids. Exemplary organic carboxylic and other acids include C₁₋₁₆ alkyl, C₆₋₁₆ aryl carboxylic acids and C₄₋₁₆ heteroaryl carboxylic acids such as acetic, glycolic, lactic, pyruvic, malonic, glutaric, tartaric, citric, fumaric, succinic, malic, maleic, oxalic, hydroxymaleic, benzoic, hydroxybenzoic, phenylacetic, cinnamic, salicylic, nicotinic, 2-phenoxybenzoic, methanesulfonic, pamoic, propionic, toluenesulfonic and trifluoroacetic acids.

Invention salts include those made from inorganic acids, e.g., HF, HCl, HBr, Hl, H₂SO₄, H₃PO₄, Na₂CO₃, K₂CO₃, CaCO₃, MgCO₃ and NaClO₃. Suitable anions, which are optionally present with a cation such a Ca⁺⁺, Mg⁺⁺, Li⁺, Na⁺ or K⁺, include arsenate, arsenite formate, sorbate, chlorate, perchlorate, periodate, dichromate, glycodeoxycholate, cholate, deoxycholate, desoxycholate, taurocholate, taurodeoxycholate, taurolithocholate, tetraborate, nitrate, nitrite, sulfite, sulfamate, hyposulfite, bisulfite, metabisulfite, thiosulfate, thiocyanate, silicate, metasilicate, CN⁻, gluconate, gulcuronate, hippurate, picrate, hydrosulfite, hexafluorophosphate, hypochlorite, hypochlorate, borate, metaborate, tungstate and urate.

Salts also include the F1C salts with one or more amino acids. Many amino acids are suitable, especially the naturally-occurring amino acids found as protein components, although the amino acid typically is one bearing a side chain with a basic or acidic group, e.g., lysine, arginine, histidine or glutamic acid, or a neutral group such as glycine, serine, threonine, alanine, isoleucine, or leucine.

The invention compositions include F1Cs, their hydrates and the compounds in their ionized, un-ionized, as well as zwitterionic form. Hydrates include hemihydrates, monohydrates, dihydrates, trihydrates and tetrahydrates. Thus, for any F1Cs or compounds described herein with any substituent that contains a moiety that is partially or completely ionizable, e.g., a carboxyl group, the ionizable atom, usually hydrogen, may be replaced with one or more suitable counter ions such as a monovalent metal, a multivalent metal, an alkaline metal, or an ionizable organic moiety, e.g., Li⁺, Na⁺, K⁺, Ca⁺², Mg⁺², SO₄⁻², PO₄⁻², CH₃C(O)O—, CF₃C(O)O⁻, F⁻, Cl⁻, Br⁻, I⁻, NH₄⁺, N⁺(CH₃)₄, N⁺(C₂H₅)₄, HN⁺(C₂H₅)₃, H₂N⁺(C₂H₅)₂, β-hydroxyethyltrimethylammon, piperazinium, pyridinium, N-methylpyridinium, morpholimium, N,N-dimethylmorpholinium, p-toluidinium or another ionizable moiety described herein. When a F1C is under conditions, e.g., in a solution, where such moieties can partially or completely ionize, the ionizable moiety may be partially or completely charged, e.g., —C(O)—O—, —NH₃⁺, —C(O)—NH₃⁺ or —O—S(O)(O)—O⁻ may be partially for fully ionized.

The F1Cs include enriched or resolved optical isomers at any or all asymmetric atoms as are apparent from the depictions or are included in the compound structures. Both racemic and diasteromeric mixtures, as well as the individual optical isomers can be isolated or synthesized so as to be substantially free of their enantiomeric or diastereomeric partners, and these are all within the scope of the invention. Chiral centers may be found in F1Cs at, for example, one or more of R¹, R², R³, R⁴ or R¹⁰.

In the F1Cs, each variable group, e.g., R¹, R², R³, R⁴ or R¹⁰, is independently selected. In some embodiments one of the R¹, R², R³, R⁴, R¹⁰ at the 2, 11 and 15 positions is hydrogen and the other is —H another moiety, but usually 2, 3, 4, 5 or 6 of the remaining variable groups are not —H, i.e., they are another moiety as defined for those groups. In other embodiments, both R¹, R², R³, R⁴, R¹⁰ at the 2, 11 and 15 positions, are independently selected moieties other than hydrogen, i.e., they are another moiety as defined for those groups such as a C1-C20 organic moiety or C1-C20 optionally substituted alkyl group. In many embodiments R¹ at the 1-position in the β-configuration or R¹ at the 1-position in the α-configuration is not —H and R⁴ at the 1-position in the β-configuration or R¹ at the 1-position in the α-configuration is not —H.

F1Cs include compounds having structure 2 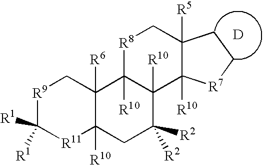
2 wherein there are 0, 1, 2, 3, 4 or 5 double bonds in the steroid rings at the 1-, 2-, 3-, 4-, 5-, 5(10), 6-, 7-, 8-, 8(14)-, 9-, 9(14)-, 11-, 12-, 13(17)-, 14-, 15- or 16- positions; and one or two independently selected R¹⁰ moieties is optionally present at 1, 2, 3 or more of the 1-, 2-, 4-, 6-, 11-, 12- or 14-positions; each variable group is independently chosen and has the meaning given above; and D is a heterocycle, a 4-, 5-, 6- or 7-membered carbon ring, or two fused rings, each being 4-, 5-, 6- or 7-membered carbon ring, wherein 1, 2 or 3 ring carbon atoms of the 4-, 5-, 6- or 7-membered carbon ring(s) are optionally independently substituted with 1 or 2 independently selected R¹⁰ moieties. In some embodiments, the D structure comprises two 5- or 6-membered rings, wherein the rings are fused or are linked by 1 or 2 bonds, optionally wherein 0, 1, 2 or 3 of R⁷, R⁸ and R⁹ are not —CHR¹⁰— or —C(R¹⁰)₂—.

Exemplary F1C of structure 2 include the following structures, 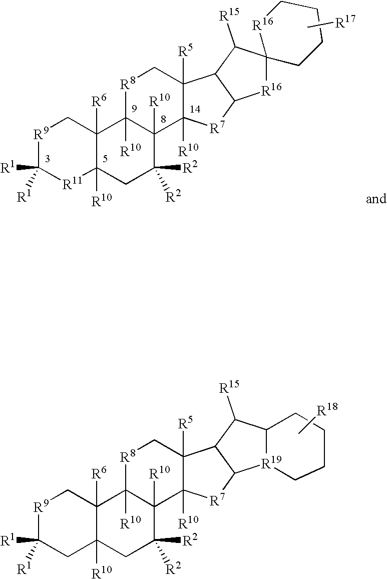
wherein, R¹⁶ independently are —CH₂—, —O—, —S— or —NH—; R¹⁵, R¹⁷ and R¹⁸ are independently selected R¹ moieties, e.g., —H, —OH, —OR^{PR}, ═O, —SR^{PR}, ═S, —O—Si—(R¹³)₃, ester, ether, acyl, halogen or optionally substituted alkyl; and R¹⁹ is nitrogen or CH; one or two independently selected R¹⁰ moieties are optionally present at one or two of the 1-, 6- and 12-positions and other variable groups are as described above. For F1Cs of structure 2 where two variable groups are bonded to the same carbon, e.g., R¹ at the 3-position, R² at the 7-position or R¹⁰ at the 11-position, the each variable group at that position is independently selected. As shown in the structure, the R¹⁷ moiety can be bonded to the ring carbon adjacent to R¹⁶, or it can be bonded to the adjacent 1, 2 or 3 ring carbons. Similarly, the R¹⁸ moiety can be bonded to the ring carbon adjacent to R¹⁹, or it can be bonded to the adjacent 1, 2 or 3 ring carbons. Structure 2 F1Cs can have 1, 2, 3 or 4 of R^{10A}, R^{10B}, R^{10C} and R^{10D} as —H, but usually 2 or 3 of R^{10A}, R^{10B}, R^{10C} and R^{10D} are —H.

Structure 2 compounds include structures wherein one, two or three of R⁷, R⁸ and R⁹ are independently —O—, —S—, or —NH— or wherein one or both of R⁵ and R⁶ independently are —H, —CH₃, —CH₂OR^{PR}, —CH₂OH, —CH₂SH, —CH₂SR^{PR}, —CH₂O—C(O)—C₁₋₁₀ alkyl, —CH₂S—C(O)—C₁₋₁₀ alkyl, —CH₂O—C(O)—C₁₋₁₀ alkenyl, —CH₂S—C(O)—C₁₋₁₀ alkenyl, —CH₂O—C(O)—C₀₋₄ alkyl-heterocycle, —CH₂S—C(O)—C₀₋₄ alkyl-heterocycle, —CH₂O—C(O)—C₀₋₄ alkyl-phenyl, —CH₂S—C(O)—C₀₋₄ alkyl-phenyl, wherein any C₁₋₁₀ alkyl, heterocycle or phenyl moiety is optionally substituted with one or more substituents, wherein the one or more substituents are one, two, three or more independently selected —O—, ═O, —OR^{PR}, —S—, ═S, —SR^{PR}, —NH—, —N(R^{PR})₂ or —C(O)—NH—, wherein each R^{PR} independently is —H or a protecting group.

The structure 2 compounds include 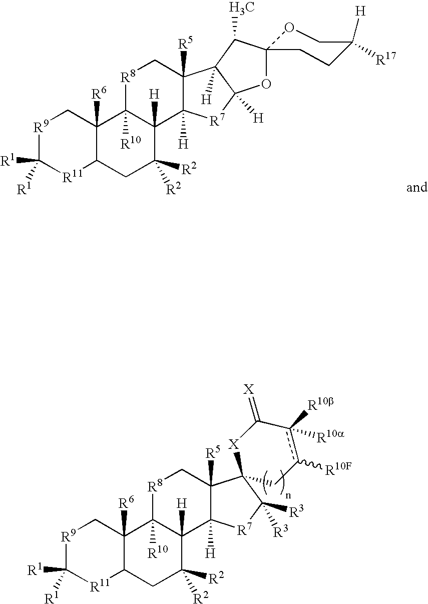
where X independently are O or S, e.g., both X can be O, R^{10α} is an independently selected R¹⁰ moiety in the α-configuration, or if a double bond is present, R^{10α} is absent, R^{10β} is an independently selected R¹⁰ moiety in the β-configuration, R^{10F} is an independently selected R¹⁰ moiety in the α- or β-configuration, n is 0, 1 or 2, and remaining variable groups are as defined above. These compounds include ones where R¹ in the α- and β-configurations independently are an R¹ moiety such as H, OH, halogen, an optionally substituted monosaccharide, an optionally substituted disaccharide or a dicarboxylic acid ester such as —OC(O)—(CH₂)₂—COOH, —OC(O)—(CH₂)₃—COOH or —OC(O)—(CH₂)₄—COOH, R² in the α- and β-configurations independently are an R² moiety such as —H, —OH, ═O, —SH, ═S, halogen, optionally substituted alkyl, a monosaccharide or a disaccharide, R⁵ is C1-C4 alkyl, R⁶ is —H, halogen or C1-C4 alkyl or R⁷ and R⁸ independently are moieties as previously defined such as independently selected —CH₂—, —CH(α-OR^{PR})—, —CH(β-OR^{PR})—, —C(O)— or —O—, R⁹ is a moiety as previously defined such as —CH₂—, —CH(α-halogen)-, —CH(α-OH)—, —CH(α-optionally substituted alkyl)-, —C(halogen)₂-, —C(β-optionally substituted alkyl)(α-OH)—, —CH(α-optionally substituted alkyl)-, R¹⁰ at the 9-position is a R¹⁰ moiety such as —H, —F, —Cl, or optionally substituted alkyl, R^{PR} is —H or a protecting group such as an ester or optionally substituted alkyl and other variable groups are as previously defined. For any of these compounds, 1, 2, 3 or 4 of R^{10A}, R^{10B}, R^{10C} and R^{10D} may be substituted, or they all be —H, while R¹⁷ may be a moiety defined previously such as C1-C6 optionally substituted alkyl, e.g., —CH₃ or —C₂H₅.

F1Cs that comprise a hydrolyzable or removable moiety(ies) may include one or more independently chosen —O—CHR²⁴C(O)OR²⁵, —S—CHR²⁴C(O)OR²⁵, —NH—CHR²⁴C(O)OR²⁵, —O—CHR²⁴C(S)OR²⁵, —S—CHR²⁴C(S)OR²⁵, —NH—CHR²⁴C(S)OR²⁵, —O—CHR²⁴OC(O)R²⁵, —S—CHR²⁴OC(O)R²⁵, —NH—CHR²⁴OC(O)R²⁵, —O—CHR²⁴C(O)N(R²⁵)₂, —S—CHR²⁴C(O)N(R²⁵)₂, —NH—CHR²⁴C(O)N(R²⁵)₂, —O—CHR²⁴OR²⁵, —S—CHR²⁴OR²⁵, —NH—CHR²⁴OR²⁵, —O—CHR²⁴C(R²⁵)₂CH₂OX, —S—CHR²⁴C(R²⁵)₂CH₂OX, —NH—CHR²⁴C(R²⁵)₂CH₂OX, —O—CHR²⁴C(R²⁵)₂OX, —S—CHR²⁴C(R²⁵)₂OX or —NH—CHR²⁴C(R²⁵)₂OX, groups that one or more of the variable groups can comprise, e.g., R¹—R⁶, R¹⁰, R¹⁵, R¹⁷ or R¹⁸. For these hydrolyzable moieties, R²⁴ independently is —H, —CH₂—C₆H₅, —CH₂CH₂—C₆H₅, C₁₋₈ alkyl, C₂₋₈ alkenyl, aryl or heterocycle where each alkyl, alkenyl, aryl and heterocycle moiety is independently optionally substituted with 1, 2, or 3, usually 1, —O—, —S—, —NH—, halogen, aryl, —OX, —SX, —NHX, ketone (═O) or —CN moieties or the C₁₋₈ alkyl is optionally substituted with 3, 4, 5 or 6 halogens, and X is —H or a protecting group. Exemplary R²⁴ are —H, —CH₃, —C₂H₅, —C(CH₃)₃, —CH₂—C₁₋₅ optionally substituted alkyl, —CH₂CH₂—C₁₋₄ optionally substituted alkyl and —CH₂CH₂—O—C₁₋₄ optionally substituted alkyl. R²⁵ independently is —H or a C₁₋₃₀ organic moiety such as —CH₂—C₆H₅, —CH₂CH₂—C₆H₅, C₁₋₁₂ alkyl, C₂₋₁₂ alkenyl, C₂₋₁₂ alkynyl, aryl, a heterocycle, —CH₂—heterocycle or —CH₂-aryl, where each alkyl, alkenyl, alkynyl, aryl, heterocycle, —CH₂-heterocycle or —CH₂-aryl moiety is independently optionally substituted with 1 or 2, usually 1, —O—, —S—, —NH—, halogen, aryl, —OX, —SX, —NHX, ketone (═O), —C(O)OX or —CN moieties or the C₁₋₁₂ alkyl, C₂₋₁₂ alkenyl or aryl, are optionally independently substituted with 3, 4, 5 or 6 halogens, where X is —H or a protecting group, or the aryl, heterocycle, —CH₂-heterocycle or —CH₂-aryl moieties are optionally independently substituted with 1, 2 or 3 C₁₋₄ alkyl moieties or with 1, 2 or 3 C₁₋₄ alkoxy moieties at the aryl moiety or at the heterocycle, usually at a ring carbon. Exemplary R²⁵ are —H, —CH₃, —C₂H₅, —C₃H₇, —C₄H₉, —C₆H₁₃, —C₆H₅, —C₆H₄OH, —C₆H₄OCH₃, —C₆H₄F, —CH₂—C₁₋₅ optionally substituted alkyl, —CH₂CH₂—(S)₀₋₁—C₁₋₄ optionally substituted alkyl and —CH₂CH₂—O—C₁₋₄ optionally substituted alkyl.

For any F1C structure, whenever a variable moiety such as R⁷, R⁸ or R⁹ or a substitution at a variable group includes moieties such as —O—CHR¹⁰—, —NR^{PR}—CHR¹⁰—, or ═N— it is intended that such moieties can be present in either orientation relative to the other ring atoms that may be present, i.e., —O—CHR¹⁰—, —NR^{PR}—CHR¹⁰—, —CHR¹⁰—O—, —CHR¹⁰—NR^{PR}, ═N— and —N═ are all included, unless defined or implied otherwise by the structure.

Invention embodiments include a composition comprising a F1C and 1, 2, 3, 4 or more nonaqueous liquid excipients. These compositions can contain less than about 3% w/v water, less than about 2% w/v water, less than about 1.5% w/v water, less than about 1% w/v water, less than about 0.8% w/v water, less than about 0.5% w/v water, less than about 0.3% w/v water or less than about 0. 1% w/v water. Typically, the nonaqueous liquid excipients include propylene glycol and a PEG or a PEG mixture and can optionally include one or both of benzyl alcohol and benzyl benzoate.

Embodiments of F1Cs include or exclude any subset of compounds within the definition of formula 1, provided that at least one F1C remains. For example, a subset of F1Cs that are may be included, for example in the invention nonaqueous formulations and in the invention intermittent dosing protocols and immune modulation methods, are (1) F1Cs where R² is hydroxyl, or a group that can hydrolyze or metabolize to hydroxyl or thiol, in either configuration and R⁵ and R⁶ are methyl in the α-configuration or (2) any 1, 2, 3, 4, 5, 6 or more of the F1Cs or genera of compounds that are disclosed herein. Another group of compounds that are optionally excluded from F1Cs comprises one or all compounds that are disclosed in one or more prior art references or publications, e.g., one or more compounds that are disclosed in one or more of the references cited herein, especially for those compounds that can render any claim or embodiment unpatentable for novelty, obviousness and/or inventive step reasons.

F1C structures include 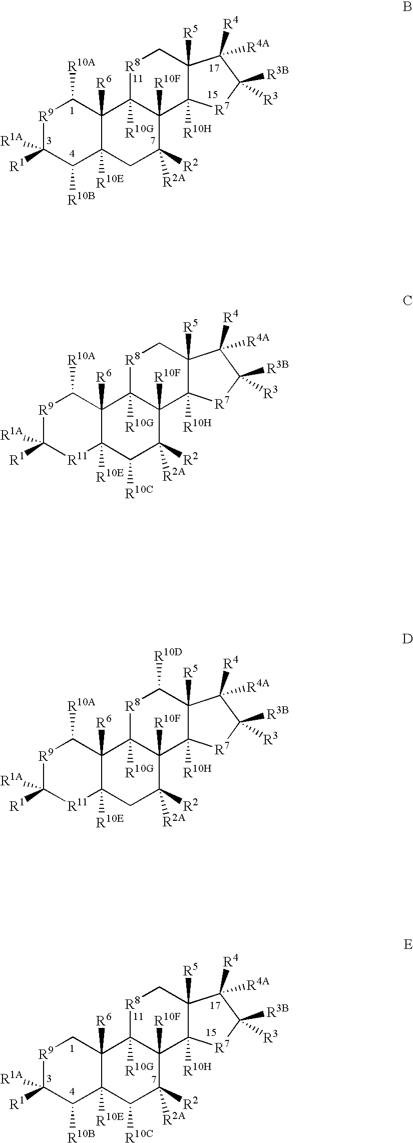 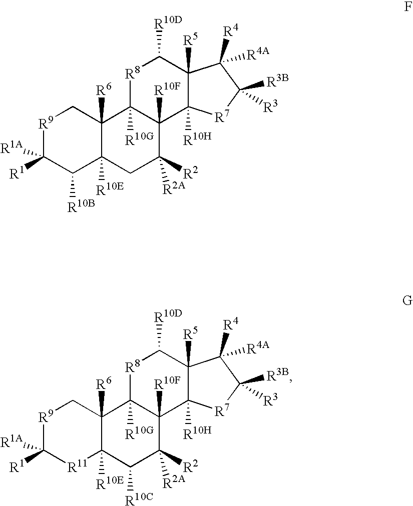
wherein 0, 1, 2, 3, 4, 5 double bonds are present in the steroid rings; R^{10E}, R^{10F}, R^{10G} and R^{10H} are independently selected R¹⁰ moieties respectively in the α,β,α,α configurations and R^{10A} and R^{10B} or R^{10A} and R^{10C} or R^{10A} and R^{10D} or R^{10B} and R^{10C} or R^{10B} add R^{10D} or R^{10C} and R^{10D} are both in α-configurations.

Similarly, when R^{10E}, R^{10F}, R^{10G} and R^{10H} respectively are in the α,β,α,α configurations and R^{10A} and R^{10B} or R^{10A} and R^{10C} or R^{10A} and R^{10D} or R^{10B} and R^{10C} or R^{10B} and R^{10D} or R^{10C} and R^{10D} respectively are in the β,α configurations exemplary B, C, D, E, F and G structures include 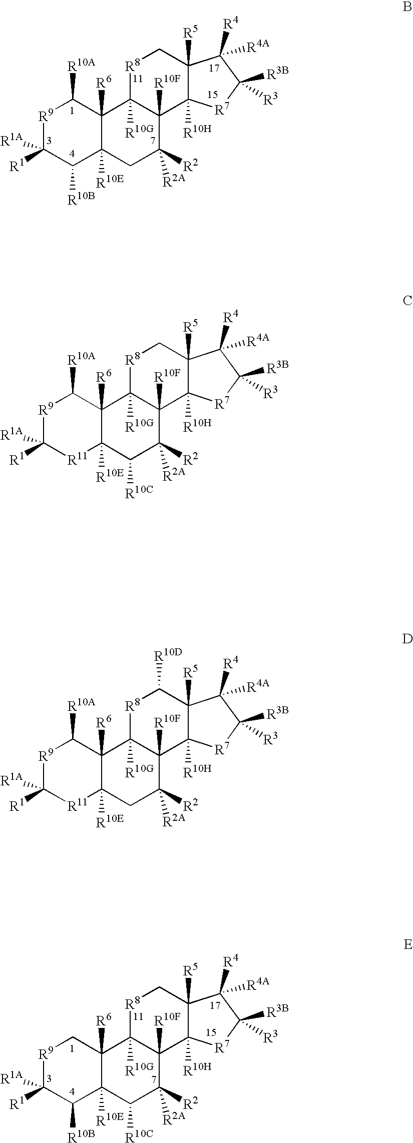 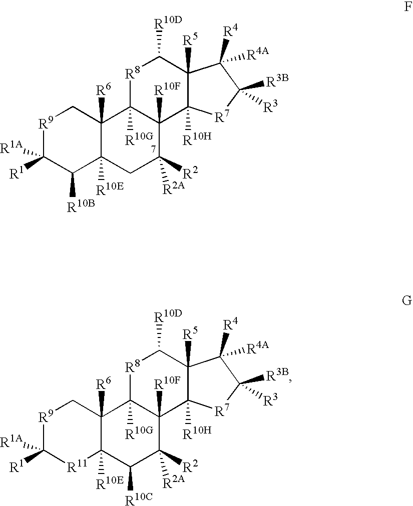
wherein 0, 1, 2, 3 or 4 double bonds are present in the steroid rings.

When R^{10E}, R^{10F}, R^{10G} and R^{10H} respectively are in the β,β,α,α configurations exemplary B, C, D, E, F and G structures include 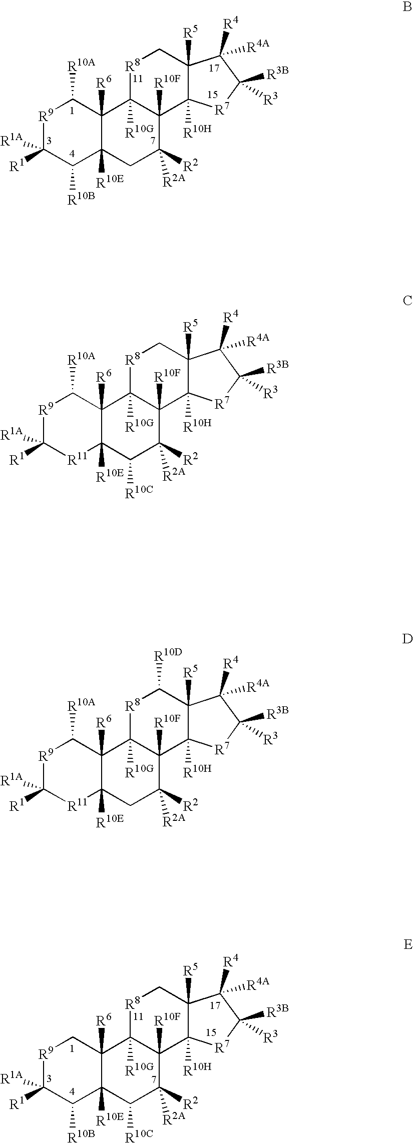 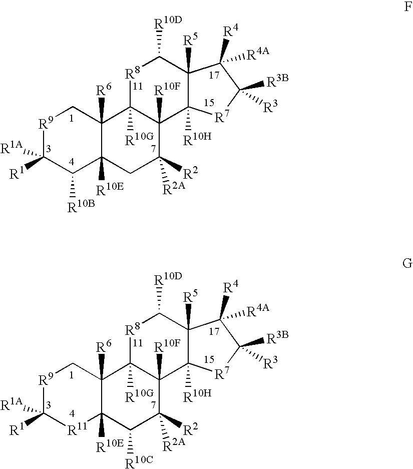 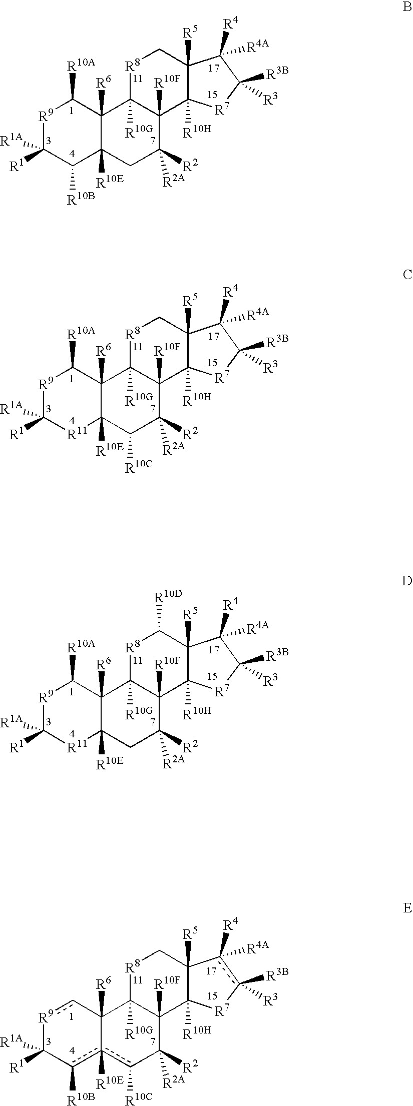 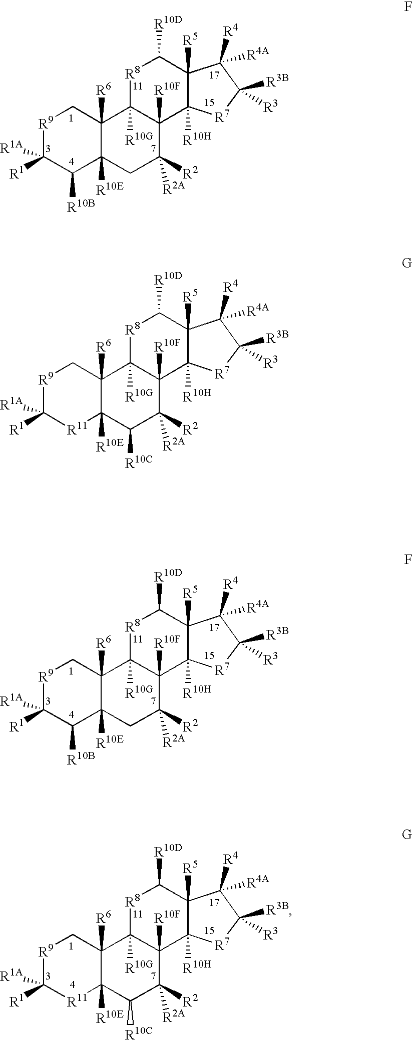
wherein 0, 1, 2, 3, 4, 5 double bonds are present in the steroid rings.

When R⁶ and R^{10C} are linked through a —CH₂—O— moiety there is no double bond at the 5-6 position and exemplary F1C structures include 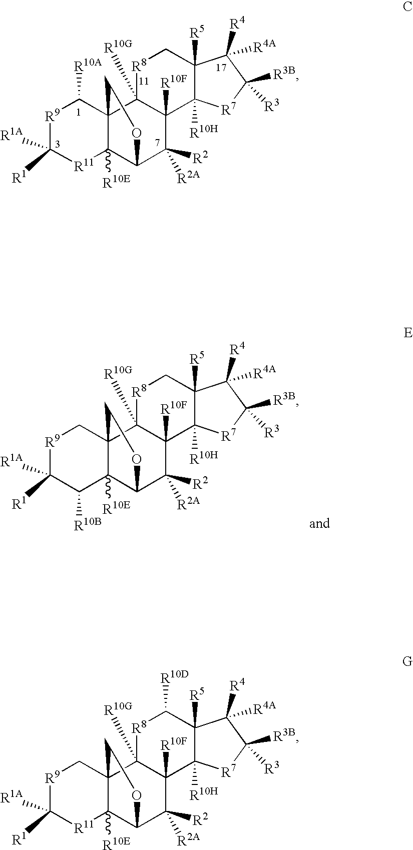
wherein 0, 1, 2, 3, 4, 5 or more double bonds are present in the steroid rings.

When adjacent variable groups are an epoxide or anoptionally substituted cyclopropyl ring exemplary F1C structures include

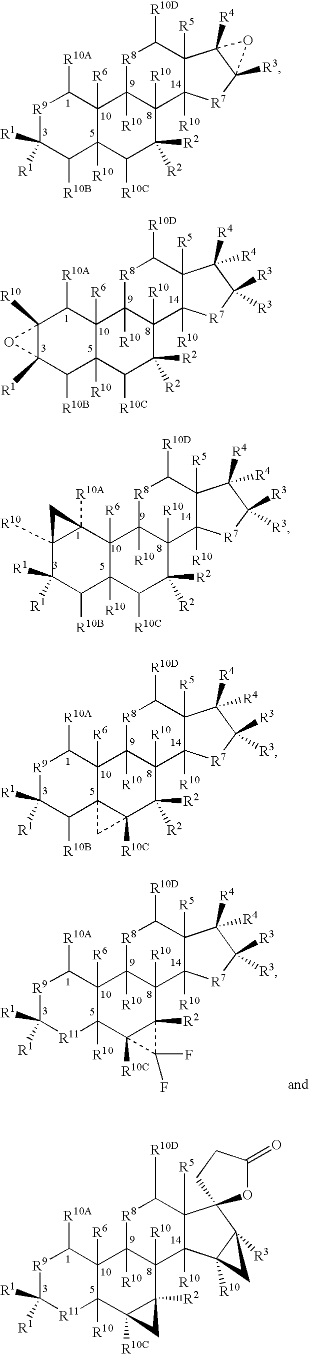

wherein 0, 1, 2, 3 or 4 double bonds are present in the steroid rings and wherein variable groups are independently selected and, when not specified otherwise, are in the α- or β-configuration. Substituents at the cyclopropyl ring include one or two halogen atoms, e.g., dichloro, dibromo or difluoro. Typically these F1C contain one or two epoxide or cyclopropyl moieties.

Other F1Cs and structures having B, C, D, E, F and G structures are apparent from the foregoing descriptions and variable group definitions.

For these exemplary F1C structures variable group is an independently selected atom or moiety as described herein, e.g., —H, —OH, ═O, —SH, ═S, —F, —Cl, —Br, —I, —CN, —SCN, —N₃, —NH—C1-C8 optionally substituted alkyl, —N(C1-C8 optionally substituted alkyl)₂ where each optionally substituted alkyl moiety is the same or different, protected ketone, e.g., ethylene ketal (—O—CH₂—CH₂—O—), —NO₂, —ONO₂, —(CH₂)ₙ—CH(O), —(CH₂)ₙ—COOH, —(CH₂)ₙ—COOR^{PR}, —(CH₂)ₙ—NHCH₃, —(CH₂)ₙ—NHR^{PR}, —(CH₂)ₙ—CH(S), —O—S(O)(O)—OH, —O—P(O)(OH)—OH, where n is 0, 1, 2, 3, 4, 5 or 6, —O-β-D-glucopyranosiduronate, —OP(O)(OH)—NH—C(═NH)—N(CH₃)—CH₂—C(O)OH, or a group such as:

optionally substituted alkyl, e.g., —CH₃, —C₂H₅, —CH₂CH₂CH₃, —CH₂(CH₃)₂, —CH₂CH₂(CH₃)₂, —CH₂CH₂CH₂CH₃, —CH₂CH₂OCH₂CH₃, —CH₂OH, —CH₂CH₂OH, —CHOHCH₃, —CH(OC(O)CH₃)—CH₃, —CH(OR^{PR})—CH₃, —CHOH—(CH₂)ₙ—OH, —CH(OR^{PR})—(CH₂)ₙ—OR^{PR}, —CHOH—(CH₂)ₙ—CH₂OH, —CH(OR^{PR})—(CH₂)n-CH₂OR^{PR}, —CHOH—(CH₂)ₙ—CH₂SH, —CH(OR^{PR})—(CH₂)ₙ—CH₂SR^{PR}, —CH₂—(CH₂) —OCH₃, —CF₃, —(CH₂)—CF₃, —(CH₂)ₜ—NH₂, —(CH₂)₂—NH₂, —(CH₂)₃—NH₂, —CH₂—NHCH₃, —(CH₂)₂—NHCH₃, —(CH₂)ₜ—N(CH₃)₂, —(CH₂), —CH₂OH, —(CH₂)ₙ—CH₂F, —(CH₂)ₙ—CH₂Cl, —(CH₂)ₙ—CH₂Br, —CH(CH₃)—(CH₂)₃—CH(CH₃)₂, —CH(CH₃)—(CH₂), —CH(CH₃)₂, —CH(CH₃)—(CH₂)₃—CH(CH₃)—CH₂OH, —CH(CH₃)—(CH₂)ₙ—CH(CH₃)—CH₂OH, —CH(CH₃)—(CH₂)₃—CH(CH₃)—CH₂F, —CH(CH₃)—(CH₂)ₙ—CH(CH₃)—CH₂F, —CH(CH₃)—(CH₂)₃—CH(CH₃)—CH₂Cl, —CH(CH₃)—(CH₂)ₙ—CH(CH₃)—CH₂Cl, —CH(CH₃)—(CH₂)₃—CH(CH₃)—CH₂Br, —CH(CH₃)—(CH₂)ₙ—CH(CH₃)—CH₂Br, —CH(CH₃)—(CH₂)₃—CH(CH₂F)₂, —CH(CH₃)—(CH₂)ₙ—CH(CH₂F)₂, —(CH₂)₃—CH(CH₃)₂, —(CH₂)ₙ—CH(CH₃)₂, —(CH₂)₃—CH(CH₃)—CH₂OH, —(CH₂)ₙ—CH(CH₃)—CH₂OH, —(CH₂)₃—CH(CH₃)—CH₂F, —(CH₂)ₙ—CH(CH₃)—CH₂F, —(CH₂)₃—CH(CH₃)—CH₂Cl, —(CH₂)ₙ—CH(CH₃)—CH₂Cl, —(CH₂)₃—CH(CH₃)—CH₂Br, —(CH₂)ₙ—CH(CH₃)—CH₂Br, —(CH₂)₃—CH(CH₂F)₂, —CH₃, —CH₂CH₃, —CH₂CH₂CH₃, —CH₂OH, —CH₂CH₂OH, —CH₂CH₂CH₂OH, —CH₂CH(OH)CH₃, —(CH₂)ₙ—CH(CH₂F)₂, —CH(CH₃)—(CH₂)ₙ—CH(CH₂CH₃)—CH(CH₃)ₘ(CH₂R⁵¹)ₚ, —C≡CH, —C≡CCH₃, —C≡CCF₃, —C≡CCl, —CH═CH₂, —CF═CF₂, —CF═CFCH₃, —CH═CHCH₃, —C(O)—NH—C₆H₅, —C(O)—NH—CH₃, —C(O)—NH—C₂H₅, —C(CH₃)═N—OH, —C(CH₃)═N—NH—C(O)—OC₂H₅, —C(CH₃)═N—NH—C(O)—OC₄H₉, —C(CH₃)═N—NH—C(O)—OC₆H₅, —CH₂—C₆H₅, —CH₂—C₆H₅(CH₂)ₙ—F, —C₆H₅, —C₆H₄(CH₂)ₙ—OH, —(CH₂)ₚ—CH=[z]CH—(CH₂)ₘ—C₆H₄-o-NH₂ (where o means ortho substituted), —(CH₂)ₚ—CH=[E]CH—(CH₂)ₘ—C₆H₄-o-NH₂, —(CH₂)ₚ—CH=[z]CH—(CH₂)ₘ—C₆H₄-m-NH₂ (where m means meta substituted), —(CH₂)ₚ—CH=[E]CH—(CH₂)ₘ—C₆H₄-m-NH₂, —(CH₂)ₚ—CH=[z]CH—(CH₂)ₘ, C₆H₄-p-NH₂ (where p means para substituted), —(CH₂)ₚ—CH=[E]CH—(CH₂)ₘ—C₆H₄-P-NH₂, —(CH₂)ₚ—CH=[z]CH—(CH₂)ₘ—C₆H₄-o-NHCH₃, —(CH₂)ₚ—CH=[E]CH—(CH₂)ₘ—C₆H₄-o-NHCH₃, —(CH₂)ₚ—CH=[z]CH—(CH₂)ₘ—C₆H₄-m-NHCH₃, —(CH₂)ₚ—CH=[E]CH—(CH₂)ₘ—C₆H₄-m-NHCH₃, —(CH₂)ₚ—CH=[z]CH—(CH₂)ₘ—C₆H₄-p-NHCH₃—(CH2)p-CH=[E]CH—(CH₂)ₘ—C₆H₄-p-NHCH₃, (CH₂)ₚ—CH=[z]CH—(CH₂)ₘ—C₆H₄-o-NHC₂H₅—(CH₂)ₚ—CH=[E]CH—(CH₂)ₘ—C₆H₄-o-NHCH₂H₅, —(CH₂)ₚ—CH=[z]CH—(CH₂)ₘ—C₆H₄-m-NHC₂H₅, —(CH₂)ₚ—CH=[E]CH—(CH₂)ₘ—C₆H₄-m-NHC₂H₅, —(CH₂)ₚ—CH=[z]CH—(CH₂)ₘ—C₆H₄-p-NHC₂H₅, —(CH₂)ₚ—CH=[E]CH—(CH₂)ₘ—C₆H₄-p-NHC₂H₅, —(CH₂)ₚ—CH=[z]CH—(CH₂)ₘ—C₆H₄-o-N(C₂H₅)₂, —(CH₂)ₚ—CH=[E]CH—(CH₂)ₘ—C₆H₄-o-N(C₂H₅)₂, —(CH₂)ₚ—CH=[z]CH—(CH₂)ₘ—C₆H₄-m-N(C₂H₅)₂, —(CH₂)ₚ—CH=[E]CH—(CH₂)ₘ—C₆H₄-m-N(C₂H₅)₂, —(CH₂)ₚ—CH=[z]CH—(CH₂)ₘ—C₆H₄-p-N—N(C₂H₅)₂, —(CH₂)ₚ—CH=[E]CH—(CH₂)ₘ—C₆H₄-p-N(C₂H₅)₂, —(CH₂)ₚ—CH=[z]CH—(CH₂)ₘ—C₆H₄-o-N(CH₃)₂, —(CH₂)ₚ—CH=[E]CH—(CH₂)ₘ—C₆H₄-o-N(CH₃)₂, —(CH₂)ₚ—CH=[z]CH—(CH₂)ₘ—C₆H₄-m-N(CH₃)₂, —(CH₂)ₚ—CH=[E]CH—(CH₂)ₘ—C₆H₄-m-N(CH₃)₂, —(CH₂)ₚ—CH=[z]CH—(CH₂)ₘ—C₆H₄-p-N—N(CH₃)₂, —(CH₂)ₚ—CH=[E]CH—(CH₂)ₘ—C₆H₄-p-N(CH₃)₂, —(CH₂)ₚ—CH=[z]CH—(CH₂)ₘ—C₆H₄-o-NH—C1-6 optionally substituted alkyl, —(CH₂)ₚ—CH=[E]CH—(CH₂)ₘ—C₆H₄-o-NH—C1-6 optionally substituted alkyl, —(CH₂)ₚ—CH=[z]CH—(CH₂)ₘ—C₆H₄-m-NH—C1-6 optionally substituted alkyl, —(CH₂)ₚ—CH=[E]CH—(CH₂)ₘ—C₆H₄-m-NH—C1 -6 optionally substituted alkyl, —(CH₂)ₚ—CH=[z]CH—(CH₂)ₘ—C₆H₄-p-NH—C1-6 optionally substituted alkyl, —(CH₂)ₚ—CH=[E]CH—(CH₂)ₘ—C₆H₄-p-NH—C1-6 optionally substituted alkyl, —(CH₂)ₚ—CH=[z]CH—(CH₂)ₘ—C₆H₄-o-N(C1-6 optionally substituted alkyl)₂, —(CH₂)ₚ—CH=[E]CH—(CH₂)ₘ—C₆H₄-o-N(C1-6 optionally substituted alkyl)₂, —(CH₂)ₚ—CH=[z]CH—(CH₂)ₘ—C₆H₄-m-N(C1-6 optionally substituted alkyl)₂, —(CH₂)ₚ—CH=[E]CH—(CH₂)ₘ—C₆H₄-m-N(C1-6 optionally substituted alkyl)₂, —(CH₂)ₚ—CH=[z]CH—(CH₂)ₘ—C₆H₄-p-N(C1-6 optionally substituted alkyl)₂, —(CH₂)ₚ—CH=[E]CH—(CH₂)ₘ—C₆H₄-p-N(C1-6 optionally substituted alkyl)₂, —C₆H₄(CH₂), —C(O)OH, —C₆H₄(CH₂), —C(O)OCH₃, —CH═CH—(CH₂)ₙ—CH₃, —CH(CH₃)—(CH₂)ₙ—CH₂—C(H)_{q}(CH₃)ₘ(CH₂R⁵¹)ₚ, —CH(CH₃)—(CH₂)ₙ—CH═C(CH₃)(CH₂OH), —CH(CH₂OH)—(CH₂)ₙ—CH═C(CH₃)₂, ═CH—(CH₂)ₙ—R⁴⁵, ═CH—(CH₂)ₜ—(CH═CH)—R⁴⁵, ═C(CH₃)—CH₂—C(O)—N(C1-C6 alkyl)₂, ═C(CH₃)—(CH₂)₂—C(O)—N(C1-C6 alkyl)₂, ═C(CH₃)—CH₂—C(O)—NH—C1-C6 alkyl, ═C(CH₃)—(CH₂)₂—C(O)—NH—C1-C6 alkyl, ═C(CH₃)—CH₂—N(C1-C6 alkyl)₂, ═C(CH₃)—(CH₂)₂—N(C1-C6 alkyl)₂, ═C(CH₃)—CH₂—NH—C1-C6 alkyl, ═C(CH₃)—(CH₂)₂—NH—C1-C6 alkyl, ═CH—CH₂—C(O)—N(C1-C6 alkyl)₂, ═CH—(CH₂)₂—C(O)—N(C1-C6 alkyl)₂, ═CH—CH₂—C(O)—NH—C1-C6 alkyl, ═CH—(CH₂)₂—C(O)—NH—C1-C6 alkyl, ═CH—CH₂—N(C1-C6 alkyl)₂, ═CH—(CH₂)₂—N(C1-C6 alkyl)₂, ═CH—CH₂—NH—C1-C6 alkyl, ═CH—(CH₂)₂—NH—C1-C6 alkyl, ═C(CH₃)—CH₂—C(O)—NH₂, ═C(CH₃)—(CH₂)₂—C(O)—NH₂, ═C(CH₃)—(CH₂)₂—NH₂, ═C(CH₃)—CH₂—NH₂, ═CH—CH₂—C(O)—NH₂, =CH—(CH₂)₂—C(O)—NH₂, ═CH—CH₂—NH₂, ═CH—(CH₂)₂—NH₂, —(CH₂)₃—X—(CH₂)₃—C₂F₅, —(CH₂)5-N(CH3)-(CH2)3-S—(CH2)3-C2F5,—(CH2)5-NH—(CH2)3-S—(CH2)3-C2F5,—(CH2)5-N(CH3)-(CH2)3-S—CH2-2-pyridyl, —(CH2)5-N(CH3)-(CH2)3-SO—CH2-2-pyridyl, —(CH2)5-N(CH3)-(CH3)-(CH2)3-S—CH2-p-CF3-phenyl, —(CH2)5-N(CH3)-(CH2)3-SO—CH2-p-CF3-phenyl, —(CH2)5-[2-pyrrolidine-1-yl]-CH2-S-p-CF3-phenyl, —(CH2)5-[2-pyrrolidine-1-yl]-CH2-SO-p-CF3-phenyl, —(CH2)5-N(CH3)-(CH2)3C2F5, —(CH2)5-N(CH3)-(CH2)6C2F5,—(CH2)5-N(CH3)-(CH2)7-C2F5, —(CH2)5-N(CH3)-(CH2)8-C2F5, —(CH2)6-N(CH3)-(CH2)6-C2F5, —(CH2)6-N(CH3)-(CH2)7-C2F5, —(CH2)6-N(CH3)-(CH2)8-C2F5, —(CH2)5-N(CH3)-(CH2)2-C4F9, —(CH2)5-N(CH3)-(CH2)3-C6F13, —(CH2)5-N(CH3)-(CH2)3-C8F17, —(CH2)5-N(CH3)-(CH2)6-C4F9, —(CH2)5-N(CH3)-(CH2)6-C6F13, —(CH2)5-N(CH2)6-C8F17, —(CH2)5-N(CH3)H, —(CH2)5-N(CH3)(CH2)9-H, —(CH2)5-N(CH3)CH2 CH═CF—C2F5, —(CH2)5-N(CH3)CH2-CH═CF—C3F7, —(CH2)5-N(CH3)CH2 CH═CF—C5F11, —(CH2)5-N(CH3)CH2 CH═CF—C7F1 5, —(CH2)5-1-pyrrolidinyl, —(CH2)5-N(CH3)(CH2)3-O-phenyl, —(CH2)5-N(CH3)-(CH2)3-O-benzyl, —(CH2)5-N(CH3)(CH2)30(CH2)3C2F5, —(CH2)5-N(CH3)(CH2)3-CH(CH3)2, —(CH2)5-N(CH3)-(CH2)3-pyridyl, —(CH2)5-N(CH3)-(CH2)3-phenyl, —(CH2)5-N(CH3)-(CH2)3-p-tolyl, —(CH2)5-N(CH3)(CH2)3-p-ethoxyphenyl, —(CH2)5-N(CH3)(CH2)3-p-tolyl, —(CH2)5-N(CH3)-(CH2)3-p-chlorophenyl, —(CH2)5-N(CH3)-(CH2)3-O-CH2-phenyl, —(CH₂)₃—N(C₁₋₃ alkyl)-R⁴⁵, —(CH₂)₄—N(C₁₋₃ alkyl)-R⁴⁵, —(CH₂)₅—N(C₁₋₃ alkyl)-R⁴⁵, —(CH₂)₆—N(C₁₋₃ alkyl)-R⁴⁵, —(CH₂)₇—N(C₁₋₃ alkyl)-R⁴⁵, where R⁴⁵ is an R¹ substituent disclosed herein, e.g., —H, —OH, —F, —Cl, —Br, —I, —OCH₃, —C(O)OH, —C(O)OCH₃, —OR^{PR}, —SH, —SR^{PR}, —NH₂—NH—C1-C8 optionally substituted alkyl, —N(C1-C8 optionally substituted alkyl)₂ where each optionally substituted alkyl moiety is the same or different, —NHR^{PR}, R⁵¹ independently are an R¹ substituent disclosed herein, e.g., an ester, —F, —Cl, —Br, —I, alkyl (e.g., —CH₃), an ether (e.g., (—OCH₃), a thioether (e.g., (—SCH₃), an optionally substituted heterocycle, —C(O)OH, —NH₂ or —CN, X is —O— or —S—, m is 0, 1, 2 or 3, n independently are 0, 1, 2, 3, 4, 5 or 6, p is 0, 1, 2 or 3, q is 0, 1, 2 or 3, t is 1, 2, 3 4, 5 or 6 and R^{PR} are —H or independently selected protecting groups, or

optionally substituted alkenyl, e.g., ═CH₂, ═CH₂CH₃, ═CH—CH₂OH, ═CH—(CH₂)ₙ—OR^{PR}, —CH═CH₂, —CH═CHF, —CH═CHCl, —CH═CHBr, —CH═CHI, —CH═CH—(CH₂)ₙ—OH, —CH═CH—(CH₂)ₙ—F, —CH═CH—(CH₂)ₙ—Cl, —CH═CH—(CH₂)ₙ—Br, —CH═CH—(CH₂)ₙ—I, —CH═NCH₃, —CH═NR^{PR}; —CH═N—CH₃, —CH═CH—CH₃, —CH═CH—(CH₂)ₙ—COOR^{PR}, —CH═CH—(CH₂)ₙ—NHR^{PR}, —CH═CH—CH₂—OR^{PR}, —CH═CH—CH₂—CF₃, —CH═CH₂—CH₂-halogen, —CH═CH—(CH₂)ₙ—OCH₃, —CH═CH—(CH₂)ₙ—C(O)—O-optionally substituted alkyl, —CH═CH—(CH₂)ₙ—C(O)—S-optionally substituted alkyl, ═CH—CH₂—(CH₂)ₙ—SR^{PR}, ═CH—(CH₂)ₙ—C(O)NHR^{PR}, ═CH—(CH₂)ₙ—C(O)NHCH₃, ═CH(CH₂)ₙ—C(O)NHC₂H₅, ═CH—CH₂CH₃, ═CH—(CH₂)ₙ—CH(CH₃)₂, ═CH—(CH₂)ₙ—CH(CH₃)(CH₂OR^{PR}), ═CH—(CH₂)ₙ—CH(CH₃)(CH₂C(O)OR^{PR}), ═CH—(CH₂)ₙ—OH, ═CCH₃—(CH₂)ₙ—OR^{PR}, ═CCH₃—(CH₂)ₙ—C(O)OR^{PR}, ═CCH₃—(CH₂)ₙ—C(O)NHR^{PR}, ═CCH₃—(CH₂)ₙ-halogen, ═CH—CHOH—CH₂—OH or ═CH—CH₂CH₂-halogen, where R^{PR} is —H or a protecting group and n is 0, 1, 2, 3, 4, 5 or 6, or

optionally substituted alkynyl, e.g., —C≡CH, —C≡C—(CH₂)ₘ—OH, —C≡C-halogen, —C≡C—CH₃, —C═CCF₃, —C≡CCH₂F, —C≡CCH₂Cl, —C≡CCH₂Br, —C≡CCH₂I, —C≡C—CH₂OH, —C≡C—CH₂-halogen, —C≡C—CH₂—C(O)OR^{PR}, —C≡C—CH₂—CH₃, —C═CCH₂CF₃, —C≡C—CH₂—CH₂OH, —C≡C—CH₂—CH₂-halogen, —C≡C—(CH₂)ₙ—C₆H₅, —C≡C—(CH₂)ₙ—C₆H₄OH, —C≡C—(CH₂)ₙ—C₆H₄COOR^{PR}, —C═C—(CH₂)n-C₆H₃(OH)₂, —C═C—(CH₂)ₙ—C₆H₄F, —C≡C—(CH₂)ₙ—C₆H₄Br, —C≡C—CH₂—CH₂—C(O)OR^{PR}, —C≡C—(CH₂)ₙ—CH₃, —C≡C—CH(CH₃)—(CH₂)ₙ—CH₃, —C≡C—(CH₂)ₙ—CHOR^{PR}, —C≡C—CH(CH₃)—(CH₂)ₙ—CHOR^{PR}, —C≡C—CHCOOR^{PR}, —C≡C—CH(CH₃)—(CH₂)ₙ—NHR^{PR}, —C≡C—(CH₂)ₙ—NHR^{PR},—C≡C—(CH₂)ₙ—C(O)NHR^{PR}, —C≡C—(CH₂)ₙ—C(O)NH—(CH₂)ₙ—CH₃, —C≡C—C≡C—(CH₂)ₙ—CH₃, —C≡C—C≡C—(CH₂)ₙ-halogen, —C≡C—(CH₂)ₙ—OS(O)(O)—O—R^{PR}, —C≡C—(CH₂)ₙ—OS(O)(O)—O-optionally substituted alkyl, —C≡C—C≡C—(CH₂)ₙ—OR^{PR} or —C≡C—CH(CH₃)—(CH₂)ₙ—CHOR^{PR} where n is 0, 1, 2, 3, 4, 5 or 6, m is 1, 2, 3 or 4 and R^{PR} is —H or a protecting group, or

optionally substituted aryl, optionally substituted alkylaryl, optionally substituted alkenylaryl or optionally substituted alkynylaryl, e.g., optionally substituted phenyl, optionally substituted benzyl, —(CH₂)ₙ—C₆H₄OH, —(CH₂)ₙ—C₆H₄OR^{PR}, —(CH₂)ₙ—C₆H₃(OH)₂, —(CH₂)ₙ—C₆H₄F, —(CH₂)ₙ—C₆H₄Br, —(CH₂)ₙ—C₆H₄C(O)OR^{PR}, —(CH₂)ₙ—C₆H₄C(O)SR^{PR}, or analogs where the aromatic ring contains 1, 2, 3 or 4 independently chosen substituents such as independently chosen halogen, —OH, —SH, —NO₂, —CN, —SCN, —N₃, C1-C6 ester, C1-C6 alkyl, C1-C6 ether, C1-C6 thioether, —OR^{PR}, —(CH₂)ₙ—C(O)OR^{PR}, —(CH₂)ₙ—NHR^{PR}, —(CH₂)ₙ—OR^{PR} or —(CH₂)ₘ—O—(CH₂)ₘ—OR^{PR} where n is 0, 1, 2, 3, 4, 5 or 6, m independently are 1, 2 or 3 and R^{PR} independently are —H or a protecting group, or

ether, e.g., optionally substituted alkoxy, optionally substituted alkenyloxy, optionally substituted alkynyloxy, optionally substituted aryloxy, —OCH₃, —OC₂H₅, —OC₃H₇, —OC₄H₉, —OC₂H₃, —OC₃H₅, —OC₄H₇, —O—C(CH₃)₃, —OCH₂CH₂OH, —O(CH₂)₂—O—CH₃, —O(CH₂)₃—O—CH₃, —O—CH(CH₃)CH₃, —O—CH₂CH₂CH₃, —OCH₂CH₂F, —OCH₂CHF₂, —OCH₂CF₃, —OCH₂CH₂Cl, —OCH₂CH₂Br, —OCH₂CH₂I, —OCH₂CH₂CH₂F, —O—CH₂—CH(C(O)—NH—CH₂C(O)OH)—NH—C(O)—(CH₂)₂—CH(NH₂)—C(O)—OH, —O—(CH₂)₂—N⁺(CH₃)₃,), —O—(CH₂)₀₋₃—(CH═CH)₀₋₁—(CH₂)₀₋₃—CH₃, —O—(CH₂)₀₋₃—(CH═CH)—(CH₂)₀₋₃—CH₂F, —O—(CH₂)₁₋₃—(C≡C)—(CH₂)₀₋₃—CH₃, —O—(CH₂)₁₋₃—(C≡C)—(CH₂)₀₋₃—CH₂F, —O—C₆H₅, —O—CH₂—C₆H₅, —O—C1-C20 organic moiety where the organic moiety is, e.g., —CH₃, —C₂H₅, i-propyl, n-propyl, t-butyl, n-butyl, i-butyl, n-hexyl, n-octyl, n-decyl, —(CH₂)₁₋₈—OH, —CHO, —(CH₂)₁₋₈—NH₂, —(CH₂)₁₋₆—C(O)—OH, —(CH₂)₀₋₃—(CH═CH)₀₋₃—(CH₂)₀₋₃—CH₃, —(CH₂)₀₋₃—(CH═CH)₀₋₁—(CH₂)₀₋₃—CH₂F, —(CH₂)₀₋₃—(CH═CH)₀₋₁—(CH₂)₀₋₃—CH₂Br, —(CH₂)₀₋₃—(CH═CH)₀₋₁—(CH₂)₀₋₃—C(O)—OR^{PR}, —(CH₂)₀₋₃—(CH═CH )₀₋₁—(CH₂)₀₋₃—NHR^{PR}, —C(O)—CH₃, —C(O)—C₂H₅, —C(O)—C₆H₅, —CF₃, —CH₂CF₃ or —C₂F₅, wherein R^{PR} is —H or a protecting group, —O—C₁₋₁₀ optionally substituted alkyl such as i-propyl, n-propyl, t-butyl, n-butyl, n-hexyl, n-octyl, n-decyl, —(CH₂)₁₋₈—OH, —(CH₂)₁₋₈—NH₂, —(CH₂)₁₋₈—C(O)—OH, —(CH₂)₀₋₃—(CH═CH)₀₋₁—(CH₂)₀₋₃—CH₃, —(CH₂)₀₋₃—(H═CH)₀₋₁—(CH₂)₀₋₃—CH₂F, —(CH₂)₀₋₃—(CH═CH)₀₋₁—(CH₂)₀₋₃—CH₂Br, —(CH₂)₀₋₃—(CH—CH)₀₋₁—(CH₂)₀₋₃—C(O)—OR^{PR}, —(CH₂)₀₋₃—(CH═CH)₀₋₁—(CH₂)₀₋₃—NHR^{PR}, —CF₃ and —C₂F₅, wherein R^{PR} is —H or a protecting group, or

ester, e.g., —OC(O)CH₃, —OC(O)C₂H₅, —OC(O)C₂H₃, —OC(O)CH₂CH₂CH₃, —OC(O)CH(CH₃)₂, —O—C(O)—(CH₂)₂—C(O)OH, —O—C(O)—(CH₂)₂—C(O)OR^{PR}, —O—C(O)—(CH₂)₃—C(O)OH, —O—C(O)—(CH₂)₃—C(O)OR^{PR}—O—C(O)—(CH₂)₄—C(O)OH, —O—C(O)—(CH₂)₅—C(O)OH, —O—C(O)—(CH₂)₅—C(O)OR^{PR}, —O—C(O)—(CH₂)₄—C(O)OR^{PR}, —O—C(O)—(CH₂)₂—C(O)ONH₂, —O—C(O)—(CH₂)₂—C(O)ONHCH₃, —O—C(O)—(CH₂)₂—C(O)ONHC₂H₅, —O—C(O)—(CH₂)₂—C(O)ONHC₃H₇, —O—C(O)—(CH₂)₂—C(O)ONHC₃H₅, —O—C(O)—(CH₂)₂—C(O)ONHR^{PR}, —O—C(O)—(CH₂)₂—C(O)ON(R^{PR})₂, —OC(O)—C(CH₃)₂—(CH₂)ₘ—CH₃, —OC(O)—(CH₂)ₘ—CH₃, —OC(O)—CH(CH₃)—(CH₂)ₘ—CH₃, —OC(O)—C(CF₃)₂—(CH₂)ₘ—CH₃, —OC(O)—CH(CF₃)—(CH₂)ₘ—CH₃, —OC(O)C₃H₇, —OC(O)C₃H₅, —OC(O)C₄H₉, —OC(O)C₄H₇, —OC(O)C(CH₃)₃, —OC(O)CH₂CH₂CH₂CH₃, —OC(O)C₆H₅, —OC(O)CH₂C₆H₅, —OC(O)—(CH₂)₂—C(O)OH, —OC(O)—(CH₂)₂—C(O)OCH₃, —OC(O)—(CH₂)₃—C(O)OH, —OC(O)—(CH₂)₃—C(O)OCH₃, —OC(O)—(CH₂)₄—C(O)OH, —OC(O)—(CH₂)₄—C(O)OCH₃, —OC(O)—CH(CH₃)—CH₂—C(O)OH, —OC(O)—CH(CH₃)—CH₂—C(O)OCH₃, —OC(O)—CH(CH₃)—(CH₂)₂—C(O)OH, —OC(O)—CH(CH₃)—(CH₂)₂—C(O)OCH₃, —OC(O)—C(CH₃)₂—CH₂—C(O)OH, —OC(O)—C(CH₃)₂—CH₂—C(O)OCH₃, —OC(O)—C(CH₃)₂—(CH₂)₂—C(O)OH, —OC(O)—C(CH₃)₂—(CH₂)₂—C(O)OCH₃, —OC(O)—(CH₂)₂—C(O)OH, —O—C(O)—C(O)—O—(CH₂)ₘ—CH₃, —O—C(O)—C(O)—O—(CH₂)ₘ—CH2OH, —O—C(O)—(CH₂)ₙ—C(O)—O—(CH₂)ₘ—CH₃, —O—C(O)—(CH₂)ₙ—C(O)—O—(CH₂)ₘ—CH₂OH, —O—C(O)—CH(NH₂)—CH₂OH, —O—C(O)—CH₂—N(CH₃)—C(═NH)—NH₂, —O—C(O)—CH₂—NH—C(O)—CH(CH₂SH)—NH—C(O)—(CH₂)₂—CH(NH₂)—C(O)—OH, a C2-C20 ester such as —O—C(O)—CH₃, —O—C(O)—CF₃, —O—C(O)—CCL₃, —O—C(O)—C₂H₅, —O—C(O)—C₄H₇, —O—C(O)—C₆H₅, —O—C(O)—(CH₂)₂-CH₃, —O—C(O)—(CH₂)₃—CH₃, —O—C(O)—(CH₂)₄—CH₃, —O—C(O)—(CH₂)₅—CH₃, —O—C(O)—(CH₂)₆—CH₃, —O—C(O)-2 furanyl, —O—C(O)-2 thiophenyl, —o—C(O)-2 pyrrolyl, —O—C(O)-2 pyrimid inyl, —O—C(O)-3 pyrimidinyl, —O—C(O)-2 pyridyl, —O—C(O)-3 pyridyl, —O—C(O )-heterocycle, —O—C(O)—(CH₂)ₘ—C(O)O—C1-C10 optionally substituted alkyl, —O—C(O)—(CH₂)ₘ—C(O)O—C2C10 optionally substituted alkenyl, —O—C(O)—(CH₂)ₘ—O—(CH₂)ₘ—C(O)O—C1-C10 optionally substituted alkyl, —O—C(O)—(CH₂)ₘ—O—(CH₂)ₘ—C(O)OR^{PR}, —O—C(O)—(CH₂)ₘ—S——(CH₂)ₘ—C(O)O—C1-C10 optionally substituted alkyl, —O—C(O)—(CH₂)ₘ—S—(CH₂)ₘ—C(O)OR^{PR}, —O—C(O)—(CH₂)ₘ—NR^{PR}—(CH₂)ₘ—C(O)O—C1-C10 optionally substituted alkyl, —O—C(O)—(CH₂)ₘ—NR^{PR}—(CH₂)ₘ—C(O)OR^{PR}, —O—C(O)—C₁₋₁₂ optionally substituted alkyl, —OC(O)—(CH₂)_{q}—C(O)OH, —OC(O)—(CH₂)_{q}—C(O)O—C₁₋₈ optionally substituted alkyl, —OC(O)—CH(CH₃)—(CH₂)_{q}—C(O)OH, —OC(O)—CH(CH₃)—(CH₂)_{q}—C(O)O—C₁₋₈ optionally substituted alkyl, —OC(O)—C(CH₃)₂—(CH₂)_{q}—C(O)OH, —OC(O)—C(CH₃)₂—(CH₂)_{q}—C(O)O—C₁₋₈ optionally substituted alkyl, —OC(O)—C(C2H₅)(CH₃)—(CH₂)_{q}—C(O)OH, —OC(O)—C(C₂H₅)(CH₃)—(CH₂)_{q}—C(O)O—C₁₋₈ optionally substituted alkyl, —OC(O)—C(C₂H₅)₂—(CH₂)_{q}—C(O)OH, —OC(O)—C(C₂H₅)₂—(CH₂)_{q}—C(O)O—C₁₋₈ optionally substituted alkyl, —OC(O)—C(C₂H₅)(C₃H₇)—(CH₂)_{q}—C(O)OH, —OC(O)—C(C₂H₅)(C₃H₇)—(CH₂)_{q}—C(O)O—C₁₋₈ optionally substituted alkyl, where the optionally substituted alkyl optionally is methyl, ethyl, i-propyl, n-propyl, t-butyl, n-butyl, n-hexyl, n-octyl, n-decyl, vinyl, allyl, phenyl, monosubstituted phenyl, disubstituted phenyl, trisubstituted phenyl, —CH₂OH, —CH₂OR^{PR}, —CH₂F, —CF₂H, —(CH₂)ₙ—CH₃, —(CH₂)ₙ—OH, —(CH₂)ₙ—F, —(CH₂)ₙ—Br, —(CH₂)ₙ—NH₂, —(CH₂)ₙ—C(O)—OR^{PR}—(CH₂)ₙ—O—CH₃, —(CH₂)ₙ—S—CH₃, —(CH₂)ₘ—(CH═CH)ₚ—(CH₂)_{q}—CH₃, —(CH₂)ₘ—(CH═CH)ₚ—(CH₂)_{q}—CH₂F, —(CH₂)ₘ—(CH═CH)ₚ—(CH₂)_{q}—CH₂Br, —(CH₂)ₘ—(CH═CH)ₚ—(CH₂)_{q}—C(O)—OR^{PR}, —(CH₂)ₘ—(CH═CH)ₚ—(CH₂)_{q}—NHR^{PR}, —CF₃, —CH₂CF₃ or —C₂F₅, wherein R^{PR} independently are —H, a protecting group such as C1-C10 optionally substituted alkyl (e.g., —CH₃, —C₂H₅, —C₃H₆OH) or together are a protecting group, n is 1, 2, 3, 4, 5, 6, 7 or 8, m is 0, 1, 2, 3, 4, 5 or 6, p is 0 or 1 and q is 0, 1, 2, 3, 4, 5 or 6, or

acyl, e.g., —C(O)OH, —C(O)—CH₂OH, —C(O)—CH₂F, —C(O)—CH₂Cl, —C(O)—CH₂Br, —C(O)—CH₂I, —C(O)—CH₂COOH, —C(O)—CH₂COOR^{PR}, —C(O)—CH₃, —C(O)—CF₃, —C(O)—CH₂CF₃, —C(O)—CH(NH₂)—CH₂OH, —C(O)—CH₂—N(CH₃)—C(═NH)—NH₂, —C(O)—(CH₂)ₙ—CH₂OH, —C(O)—O—C(O)—C(CH₃)₃, —C(O)—O—C(O)—CH(CH₃)₂, —C(O)—O—C(O)—CH₃, —C(O)—O—C(O)—C₂H₅, —C(O)—(CH₂)ₙ—CH₂F, —C(O)—N(CH₃)₂, —C(O)—N(C₂H₅)₂, —C(O)—N(CH₃)(C₂H₅), —C(O)—NH[C(CH₃)₃], —C(O)—NH(CH₃), —C(O)NH₂, —C(O)—N(R^{PR})₂, —C(O)—(CH₂), —CH₂Cl, —C(O)—(CH₂)ₙ—CH₂Br, —C(O)—(CH₂)ₙ—CH₂—C(O)OH, —C(O)—(CH₂)ₙ—CH₂—NH₂, —C(O)—CH(CH₃)—(CH₂)₃—CH(CH₃)₂, —C(O)—CH(CH₃)—(CH₂)ₙ—CH(CH₃)₂, —C(O)—CH(CH₃)—(CH₂)₃—CH(CH₃)—CH₂OH, —C(O)—CH(CH₃)—(CH₂)ₙ—CH(CH₃)—CH₂OH, —C(O)—CH(CH₃)—(CH₂)₃—CH(CH₃)—CH₂F, —C(O)—CH(CH₃)—(CH₂)ₙ—CH(CH₃)—CH₂F, —C(O)—CH(CH₃)—(CH₂)₃—CH(CH₃)—CH₂Cl, —C(O)—CH(CH₃)—(CH₂)ₙ—CH(CH₃)—CH₂Cl, —C(O)CH₃, —C(O)CHO, —C(O)CH₂OH, —C(O)CH₂F, —C(O)CH₂Cl, —C(O)CH₂Br, —C(O)—CH₂OH, —C(O)—CH₂OR^{PR}, —C(O)—(CH₂)ₙ—CH₂OH, —C(O)—(CH₂)ₙ—CH₂OR^{PR}, —C(O)—S—(CH₂)ₙ—CH₂F, —C(O)—S—(CH₂)ₙ—CHF₂, —C(O)—S—(CH₂)ₙCF₃, —C(O)-2 furanyl, —C(O)-2 thiophenyl, —C(O)-2 pyrrolyl, —C(O)-2 pyrimidinyl, —C(O)-3 pyrimidinyl, —C(O)-2 pyridyl, —C(O)-3 pyridyl, —C(O)-heterocycle, —C(O)—C1-C10-optionally substituted alkyl, —C(O)—NH-optionally substituted phenyl, —C(O)—NH-optionally substituted heterocycle, —C(O)—(CH₂)ₙ-optionally substituted heterocycle, —C(O)—(CH₂)ₙ-optionally substituted phenyl, or —C(O)NR⁵⁰R⁵¹ where R^{PR} independently are —H or a protecting group such as C1-C10 optionally substituted alkyl, m is 0 or 1, n is 0, 1, 2, 3, 4, 5 or 6, and R⁵⁰ and R⁵¹ independently are —H, optionally substituted phenyl, optionally substituted phenylalkyl, optionally substituted alkyl optionally substituted alkenyl, or an optionally substituted heterocycle, e.g., pyridyl, pyrrolyl, pyrimidyl, benzimidazolyl, benzoxazolyl, benzofuranyl, —CH₃, —C₂H₅, 2-, 3- or 4-fluorophenyl, 2-, 3- or 4-chlorophenyl, 2-, 3- or 4-methoxyphenyl 2-, 3- or 4-methylphenyl or 2-, 3- or 4-trifluoromethylphenyl, or

thioester, e.g., —SC(O)CH₃, —SC(O)C₂H₅, —SC(O)C₃H₇, —SC(O)C₄H₉, —SC(O)C₆H₅, —SC(O)CH₂C₆H₅, —C(O)SCH₃, —CS(O)C₂H₅, —CS(O)C₃H₇, —CS(O)C₄H₉, —CS(O)C₆H₅, —CS(O)CH₂C₆H₅, —S—C(O)—(CH₂)₂—C(O)OH, —S—C(O)—(CH₂)₂—C(O)OR^{PR}, —S—C(O)—(CH₂)₃—C(O)OH, —S—C(O)—(CH₂)₃—C(O)OR^{PR}—S—C(O)—(CH₂)₄—C(O)OH, —S—C(O)—(CH₂)₅—C(O)OH, —S—C(O)—(CH₂)₅—C(O)OR^{PR}, —S—C(O)—(CH₂)₄—C(O)OR^{PR}, —S—C(O)—CH(NH₂)—CH₂OH, —S—C(O)—CH₂—N(CH₃)—C(═NH)—NH₂, —S—C(O)—CH₂—NH—C(O)—CH(CH₂SH)—NH—C(O)—(CH₂)₂—CH(NH₂)—C(O)—OH), a C2-C20 such as —S—C(O)—CH₃, —S—C(O)—CF₃, —S—C(O)—CCl₃, —S—C(O)—C₂H₅, —S—C(O)—C₆H₅, —S—C(O)—C₆H₄—OCH₃, —S—C(O)—C₆H₄—F, —S—C(O)—C₆H₄—Cl, —S—C(O)—C₆H₄—CH₃, —S—C(O)—C₁₋₁₂ optionally substituted alkyl, —S—C(O)—CH₂—NHR^{PR}, —S—C(O)—CHOH—NHR^{PR}, —S—C(O)—CH[(CH(OH)(CH₃)]—NHR^{PR}, —S—C(O)—CH(CH₃)—NHR^{PR}, —S—C(O)—CH[(CH₂)₂C(O)OR^{PR}]—NHR^{PR}, —S—C(O)—CH(CH₂C(O)OR^{PR}—NHR^{PR}, —S—C(O)—CH[(CH₂)₄NHR^{PR}]—NHR^{PR}, —S—C(O)—CH[(CH₂)₂C(O)NHR^{PR}]—NHR^{PR}, —S—C(O)—CH(CH₂C(O)NHR^{PR})—NHR^{PR}, —S—C(O)—(CH₂)ₘ—C(O)ON(R^{PR})₂, —S—C(O)—(CH₂)ₘ—O—(CH₂)ₘ—C(O)OR^{PR}, —S—C(O)—(CH₂)ₘ—S—(CH₂)ₘ—C(O)OR^{PR}, —S—C(O)—(CH₂)ₘ—NR^{PR}(CH₂)ₘ—C(O)OR^{PR}, —S—C(O)—(CH₂)ₘ—O—(CH₂)ₘ—C(O)ON(R^{PR})₂, —S—C(O)—(CH₂)ₘ—O—(CH₂)ₘ—C(O)O—C1-C10 optionally substituted alkyl, —S—C(O)—(CH₂)ₘ—O—(CH₂)ₘ—C(O)OR^{PR}, —S—C—(O)—(CH₂)ₘ—S—(CH₂)ₘ—C(O)O—C1-C10 optionally substituted alkyl, —S—C(O)—(CH₂)ₘ—S—(CH₂)ₘ—C(O)OR^{PR}—S—C(O)—(CH₂)ₘ—NR^{PR}—(CH₂)ₘ—C(O)O—C1-C10 optionally substituted alkyl, —S—C(O)—(CH₂)ₘ—NR^{PR}—(CH₂)ₘ—C(O)OR^{PR}, where the optionally substituted alkyl optionally is methyl, ethyl, i-propyl, n-propyl, t-butyl, n-butyl, n-hexyl, n-octyl, n-decyl, vinyl, allyl, phenyl, —CH₂OH, —CH₂F, —CF₂H, —(CH₂)ₙ—CH₃, —(CH₂)ₙ—OH, —(CH₂)ₙ—F, —(CH₂)ₙ—Br, —(CH₂)ₙ—NH₂, —(CH₂)ₙ—C(O)—OR^{PR}, —(CH₂)ₙ—O—CH₃, —(CH₂)ₙ—S—CH₃, —(CH₂)ₘ—(CH═CH)ₚ—(CH₂)_{q}—CH₃, —(CH₂)ₘ—(CH═CH)ₚ—(CH₂)_{q}—CH₂F, —(CH₂)ₘ—(CH═CH)ₚ—(CH₂)_{q}—CH₂Br, —(CH₂)ₘ—(CH═CH)ₚ—(CH₂)_{q}—C(O)OR^{PR}, —(CH₂)ₘ—(CH═CH)ₚ—(CH₂)_{q}—NHR^{PR},—CF₃, —CH₂CF₃, —C₂F₅, or a thio analog of any ester moiety described herein, wherein R^{PR} independently are —H, a protecting group such as C1-C10 optionally substituted alkyl (e.g., —CH₃, —C₂H₅, —C₃H₆OH) or together are a protecting group, n is 1, 2, 3, 4, 5, 6, 7 or 8, m is 0, 1, 2, 3, 4, 5 or 6, p is 0 or 1 and q is 0, 1, 2, 3, 4, 5 or 6, or

thioether, e.g., —SCH₃, —SC₂H₅, —SC₃H₇, —SC₄H₉, —SC₂H₃, —SC₄H₇, —SCH₂CH₂OH, —S—CH₂—CH(C(O)—NH—CH₂C(O)OH)—NH—C(O)—(CH₂)₂—CH(NH₂)—C(O)—OH, —S—(CH₂)₂—N⁺(CH₃)₃,), —SCH₂CH₂F, —SCH₂CHF₂, —SCH₂CF₃, —SCH₂CH₂Cl, —SCH₂CH₂Br, —SCH₂CH₂I, —SCH₂CH₂CH₂F, —S—SCH₃, —S—SC₂H₅, —S—SC₃H₇, —S—SC₄H₉, —S—C₁₋₂₀ organic moiety, —S—S—C₁₋₂₀ organic moiety, —S—CH₂—S—C₁₋₂₀ organic moiety, —S—(CH₂)₂—S—C₁₋₂₀ organic moiety, —S—(CH₂)₂—O—C₁₋₂₀ organic moiety, —S—S—CH₃, —S—S—C₂H₅, where the organic moiety is any moiety described herein such as —CH₃, —C₂H₅, i-propyl, n-propyl, t-butyl, n-butyl, n-hexyl, n-octyl, n-decyl, —(CH₂)₁₋₈—OH, —(CH₂)₁₋₈—NH₂, —(CH₂)₁₋₈—C(O)—OH, —(CH₂)₀₋₃—(CH═CH)₀₋₁—(CH₂)₀₋₃—CH₃, —(CH₂)₀₋₃—(CH═CH)₀₋₁—(CH₂)₀₋₃—CH₂F, —(CH₂)₀₋₃—(CH═CH)₀₋₁—(CH₂)₀₋₃—CH₂Br, —(CH₂)₀₋₃—(CH═CH)₀₋₁—(CH₂)₀₋₃—C(O)—OR^{PR}, —(CH₂)₀₋₃—(CH═CH)₀₋₁—(CH₂)₀₋₃—NHR^{PR}, —C(O)—CH₃, —C(O)—C₂H₅, —C(O)—C₆H₅, —S—C₃₋₈ alkyl, —S—C₃₋₈ substituted alkyl, —CF₃, —CH₂CF₃ or —C₂F₅, wherein R^{PR} is —H or a protecting group, —S—C₁₋₁₀ optionally substituted alkyl such as i-propyl, n-propyl, t-butyl, n-butyl, n-hexyl, n-octyl, n-decyl, —(CH₂)₁₋₈—OH, —(CH₂)₁₋₈—NH₂, —(CH₂)₁₋₈—C(O)—OH, —(CH₂)₀₋₃—(CH═CH)₀₋₁—(CH₂)₀₋₃—CH₃, —(CH₂)₀₋₃—(CH═CH)O—, —(CH₂)₀₋₃—CH₂F, —(CH₂)₀₋₃—(CH═CH)₀₋₁—(CH₂)₀₋₃—CH₂Br, —(CH₂)₀₋₃—(CH═CH)₀₋₁—(CH₂)₀₋₃—C(O)—OR^{PR}—(CH₂)₀₋₃—(CH═CH)₀₋₁—(CH₂)₀₋₃—NHR^{PR}, —CF₃, —C₂F₅, wherein R^{PR} is —H or a protecting group, or

thioacyl, e.g., —C(S)—(CH₂)ₙ—CH₂OH, —C(S)—(CH₂)ₙ—CH₂F, —C(S)—(CH₂)ₙ—CH₂Cl, —C(S)—(CH₂)ₙ—CH₂Br, —C(S)—CH(CH₃)—(CH₂)₃—CH(CH₃)₂, —C(S)—CH(CH₃)—(CH₂)ₙ—CH(CH₃)₂, —C(S)—CH(CH₃)—(CH₂)₃—CH(CH₃)—CH₂OH, —C(S)—CH(CH₃)—(CH₂), —CH(CH₃)—CH₂OH, —C(S)—CH(CH₃)—(CH₂)₃—CH(CH₃)—CH₂F, —C(S)—CH(CH₃)—(CH₂)ₙ—CH(CH₃)—CH₂F, —C(S)—CH(CH₃)—(CH₂)₃—CH(CH₃)—CH₂Cl, —C(S)—CH(CH₃)—(CH₂)ₙ—CH(CH₃)—CH₂Cl, —C(S)CH₃, —C(S)CH₂OH, —C(S)CH₂F, —C(S)CH₂Cl, —C(S)CH₂Br, —C(S)-2 furanyl, —C(S)-2 thiophenyl, —C(S)-2 pyrrolyl, —C(S)-2 pyrimidinyl, —C(S)-3 pyrimidinyl, —C(S)-2 pyridyl, —C(S)-3 pyridyl, —C(S)-heterocycle, —C(S)—C1-C20-optionally substituted alkyl or a thio analog of any acyl moiety described herein, where n is 0, 1, 2, 3, 4, 5 or 6, or

optionally substituted amine, e.g., —NH₂, —NH₃⁺Cl⁻, —NH₃⁺Br⁻, —NH₃⁺I⁻, alkylamine, dialkylamine, —NH—CH₃, —N(CH₃)₂, —N⁺(CH₃)₃, —N⁺(C₂H₅)₃, —NHOH, —NHR^{PR}, —N(R^{PR})₂, —NH—C(O)CH₃, —NH—C(O)CF₃, —N(C(O)CF₃)₂, —NH—C(O)CCl₃, —N(C(O)CCl₃)₂, —NH—C(O)C₆H₅, —N(C(O)C₆H₅)₂, —NH—C₂H₅, —N(C₂H₅)₂, —NH—CH₂OH, —NH—CH₂—CH₂OH, —NH—C₃H₇, —NH—C(═NH)—N(CH₃)—CH₂—C(O)OR^{PR}, —N(CH₃)₂, —N(C₂H₅)₂, —N(CH₃)(C₂H₅)—N(CH₂OH)(CH₃), —N═C[(CH₂)ₙ—H]—OH, —NH—NH—C(O)-optionally substituted alkyl, —NH—C(NH-optionally substituted alkyl)=N-optionally substituted alkyl, —N═C[(CH₂)ₙ—H]—O-optionally substituted alkyl, —NH-organic moiety, —NH—C(O)-organic moiety, e.g., —NH—C(O)—CH₃, —NH—(CH₂)ₙ-optionally substituted phenyl, —NH-optionally substituted alkyl, —N(optionally substituted alkyl)₂, —N(C(O)-optionally substituted alkyl)₂, —NH—C(O)-optionally substituted alkyl or —NH—(CH₂)ₙ-optionally substituted alkyl, wherein any of the phenyl or alkyl moieties are the same or different and are optionally substituted with 1, 2, 3 or more independently selected with substituents described herein, e.g., —O—, —NH—, —S—, —F, —Cl, —Br, —I, —OH, —OR^{PR}, —SH, —SR^{PR}, —CH₃, —C₂H₅, —O—CH₃, —O—C₂H₅, —NO₂, —CN, —SCN, —NH₂, —C(O)OR^{PR} or —(CH₂)ₙ—C(O)—OR^{PR}, wherein n is 0, 1, 2, 3 or 4, R^{PR} independently or together are —H or a protecting group and the organic moiety is as described herein, e.g., optionally substituted alkyl or an ester, or

optionally substituted amide, e.g., —C(O)—NH₂, —C(O)—NH—C(CH₃)₃, —C(O)—NH—C(CH₂OH)(CH₃)₂, —C(O)—NH—CH₃, —C(O)—NH—(CH₂)ₘ—CH₃, —C(O)—NH—(CH₂)ₘ—NH₂, —C(O)—NH—(CH₂)ₘ—NHR^{PR}, —C(O)—NH—(CH₂)ₘ—NH—(CH₂)ₙ—CH₃, —NH—C(O)H, —NH—C(O)—CH₂—CH₂—C(O)OH, —NH—C(O)—CH₂—CH₂—C(O)OR^{PR}, —NH—C(O)—(CH₂)ₘ—C(O)OH, —NH—C(O)—(CH₂)ₘ—C(O)OR^{PR}, —NH—C(O)—CH₃, —NH—C(O)—(CH₂)ₘ—CH₃, —NH—C(O)—(CH₂)ₘ—NH₂, —NH—C(O)—(CH₂)ₘ—NHR^{PR}, —NH—C(O)—O—C(CH₃)₃, —NH—C(O)—O—CH₃, —NH—C(O)—(CH₂)ₘ—NH—(CH₂)ₙ—CH₃, —C(O)—NH-organic moiety, —C(O)—NH-optionally substituted alkyl, —C(O)—NR⁴⁹—(O)ₚ-organic moiety, —C(O)—NH—(O)ₚ—(CH₂)ₙ-optionally substituted phenyl, —C(O)—NH—(CH₂)ₙ-(O)ₚ-optionally substituted alkyl, —NH—C(O)—(O)ₚ-optionally substituted alkyl, —NH—C(S)—(O)ₚ-optionally substituted alkyl, —NH—C(O)—(S)ₚ-optionally substituted alkyl, wherein 1, 2 or more of any organic, phenyl, alkyl, alkylene, e.g., —(CH₂)—, —(CH₂)ₘ— or —(CH₂)ₙ—, methyl, ethyl, n-butyl or t-butyl, moieties are optionally substituted with 1, 2, 3, 4, 5 or more independently selected substituents described herein, e.g., —F, —Cl, —Br, —I, —OH, —CH₃, —C₂H₅, —O—CH₃, —O—C₂H₅, —NO₂, —CN, —SCN, —NH₂, —C(O)OR^{PR} or —(CH₂)₁₋₄—C(O)—OR^{PR}, where R⁴⁹ is a protecting group, an organic moiety comprising about 1-10 carbon atoms or R⁴⁹ together with the organic moiety is a protecting group and the organic group optionally is optionally substituted alkyl such as i-propyl, n-propyl, t-butyl, n-butyl, n-hexyl, n-octyl, n-decyl, —(CH₂)ₘ—OH, —(CH₂)ₘ—F, —(CH₂)ₘ—Cl, —(CH₂)ₘ—Br, —(CH₂)ₘ—NH₂, —(CH₂)ₘ—C(O)—OH, —(CH₂)ₘ—C(O)—H, —(CH₂)ₘ—C(O)—CH₃, —(CH₂)ₙ—(CH═CH)ₚ—(CH₂)ₙ—CH₃, —(CH₂)ₙ—(CH═CH)ₚ—(CH₂)ₙ—CH₂F, —(CH₂)ₙ—(CH═CH)ₚ—(CH₂)ₙ—CH₂Br, —(CH₂)ₙ—(CH═CH)ₚ—(CH₂)ₙ—C(O)—OR^{PR}, —(CH₂)ₙ—(CH═CH)ₚ—(CH₂ₙ—NHR^{PR}, —CF₃ or —C₂F₅, and R^{PR} is —H or a protecting group and wherein m independently are 1, 2, 3, 4, 5 or 6, n independently are 0, 1, 2, 3 or 4 and p is 0 or 1, or

epoxide or optionally substituted cyclopropyl, when taken together with a hydrogen at an adjacent position on the steroid nucleus, usually where the epoxide or optionally substituted cyclopropyl bonds are both in the α-configuration or the β-configuration, e.g., one or more independently selected epoxide or optionally substituted cyclopropyl ring is present at the 1-2 positions, the 2-3 positions, the 4-5 positions, the 5-6 positions, the 10-11 positions, the 11-12 positions, the 15-16 positions, the 16-17 positions, or at the 2-3 and 16-17 positions of the steroid nucleus, or

—O—Si(C1-C6 alkyl)₃ where each alkyl is independently chosen, e.g., —O—Si(CH₃)₃, —O—Si[C(CH₃)₃](CH₃)₂, —O—Si[C(CH₃)₃](C₂H₅)₂, or

phosphate, phosphate ester, phosphoester, or an ether or thioether derivative thereof, e.g., —O—P(O)(OH)—OCH₃, —O—P(O)(OH)—OC₂H₅, —O—P(O)(OH)—OC₃H₇, —O—P(O)(OH)—OCH₂CH═CH₂, —O—P(O)(OCH₃)—OCH₃, —O—P(O)(OC₂H₅)—OC₂H₅, —O—P(O)(OH)—O—(CH₂)₂—N⁺(CH₃)₃, —O—P(O)(OH)—O—(CH₂)₂—NH₂), —O—P(O)(OH)—OH, —O—P(O)(OH)—SH, —O—P(O)(OR^{PR})—OH, —O—P(O)(OR^{PR})—SH, —S—P(O)(OH)—OH, —O—P(O)(OH)—S—(CH₂)₂—NH—(CH₂)₃—NH₂, —O—P(O)(OH)—O—CH₃, —O—P(O)(OCH₃)₂, —O—P(O)(OH)—O—C₂H₅, —O—P(O)(OC₂H₅)₂, —O—P(O)(OH)—O—C₃H₇, —O—P(O)(OC₃H₇)₂, —O—P—(O)(OH)—O—CH₂—CH(O—C(O)—(CH₂)_{y}(CH═CH)_{q}(CH₂)_{y}—CH₃)—CH₂—O—C(O)—(CH₂)_{y}(CH═CH)_{q}(CH₂)_{y}—CH₃, —O—P—(O)(OH)—O—CH₂—CH(O—C(O)—(CH₂)ₓCH₃)—CH₂—O—C(O)—(CH₂)ₓCH₃), —O—P—(O)(OH)—O—CH₂—CH(O—C(O)—(CH₂)₁₄CH₃)—CH₂—O—C(O)—(CH₂)₁₄CH₃), —O—P—(O)(OH)—O—CH₂—CH(O—C(O)—(CH₂)₁₂CH₃)—CH₂—O—C(O)—(CH₂)₁₂CH₃), —O—P(O)(OH)—O-optionally substituted alkyl, —S—P(O)(OH)—O-optionally substituted alkyl, —O—P(O)(OH)—S-optionally substituted alkyl, —O—P(O)(O-optionally substituted alkyl)-O-optionally substituted alkyl, —S—P(O)(O-optionally substituted alkyl)-O-optionally substituted alkyl; —O—P(O)(O-optionally substituted alkyl)-S-optionally substituted alkyl, where the optionally substituted alkyl moieties are as described herein and are independently selected, e.g., i-propyl, n-propyl, t-butyl, n-butyl, n-hexyl, n-octyl, n-decyl, —(CH₂)ₘ—OH, —(CH₂)ₘ—F, —(CH₂)ₘ—Cl, —(CH₂)ₘ—Br, —(CH₂)ₘ—NH₂, —(CH₂)ₘ—C(O)—OH, —(CH₂)ₘ—C(O)—H, —(CH₂)ₘ—C(O)—CH₃, —(CH₂)ₙ—(O)ₚ—(CH₂)ₙ—CH₃, —(CH₂) (O)ₚ—(CH₂)ₙ—CH₂OH, —(CH₂)ₙ—(CH═CH)ₚ—(CH₂)ₙ—CH₃, —(CH₂)ₙ—(CH═CH)ₚ—(CH₂)ₙ—CH₂OH, —(CH₂)ₙ—(CH═CH)ₚ—(CH₂)ₙ—CH₂F, —(CH₂)ₙ—(CH═CH)ₚ—(CH₂)ₙ—CH₂Br, —(CH₂)ₙ—(CH═CH)ₚ—(CH₂)ₙ—C(O)—OR^{PR}, —(CH₂)ₙ—(CH═CH)ₚ—(CH₂)ₙ—NHR^{PR}, —(CH₂)ₙ—(C═C)ₚ—(CH₂)ₙ—CH₃, —(CH₂)ₙ—(C═C)ₚ—(CH₂)ₙ—CH₂OH, —(CH₂)ₙ—(C═C)ₚ—(CH₂)ₙ—CH₂F, —(CH₂)ₙ—(C═C)ₚ—(CH₂)ₙ—CH₂Br, —(CH₂)ₙ—(C═C)ₚ—(CH₂)ₙ—C(O)—OR^{PR}, —(CH₂)ₙ—(C═C)ₚ—(CH₂)ₙ—NHR^{PR}, —CF₃ or —C₂F₅, wherein R^{PR} is —H or a protecting group, m is 1, 2, 3, 4, 5 or 6, n independently are 0, 1, 2, 3, 4, 5 or 6 and p is 0 or 1, q is 0 or 1, x independently are 0, 1, 2, 3, 4, 5, 6, 7, 8, 9, 10, 11, 12, 13, 14, 15, 16 or 17, y independently are 0, 1, 2, 3, 4, 5, 6, 7, 8 or 9 and substituents bonded at double bonds are in the cis, trans or mixed cis and trans configuration, wherein In some embodiments, both n and p are 1 or p is 1 and both n are 2 or one n is 1, the other n is 2 and p is 1, or

thionoester, e.g., a C2-C20 thionoester such as —O—C(S)—CH₃, —O—C(S)—CF₃, —O—C(S)—C₂H₅ or —O—C(S)—C₁₋₁₂ optionally substituted alkyl where the optionally substituted alkyl optionally is i-propyl, n-propyl, t-butyl, n-butyl, n-hexyl, n-octyl, n-decyl, vinyl, allyl, phenyl, —CH₂OH, —CH₂F, —CF₂H, —(CH₂)ₙ—CH₃, —(CH₂)ₙ—OH, —(CH₂)ₙ—F, —(CH₂)ₙ—Br, —(CH₂)ₙ—NH₂, —(CH₂)ₙ—C(O)—OR^{PR}, —(CH₂)ₙ—O—CH₃, —(CH₂)ₙ—S—CH₃, —(CH₂)ₘ—(CH═CH)ₚ—(CH₂)_{q}—CH₃, —(CH₂)ₘ—(CH═CH)ₚ—(CH₂)_{q}—CH₂F, —(CH₂)ₘ—(CH═CH)ₚ—(CH₂)_{q}—CH₂Br, —(CH₂)ₘ—(CH═CH)ₚ—(CH₂)_{q}—C(O)—OR^{PR}, —CF₃, —CH₂CF₃ or —C₂F₅, wherein R^{PR} is —H or a protecting group, n is 1, 2, 3, 4, 5, 6, 7 or 8, m is 0, 1, 2, 3, 4, 5 or 6, p is 0 or 1 and q is 0, 1, 2, 3, 4, 5 or 6, or

amino acid or peptide, e.g., a dipeptide, —O—C(O)—CH₂—NHR^{PR}, —O—C(O)—CHOH—NHR^{PR}, —O—C(O)—CH[(CH(OH)(CH₃)]—NHR^{PR}, —O—C(O)—CH(CH₃)—NHR^{PR}, —O—C(O)—CH[(CH₂)₂C(O)OR^{PR}]—NHR^{PR}, —O—C(O)—CH(CH₂C(O)OR^{PR}—NHR^{PR}, —O—C(O)—CH[(CH₂)₄NHR^{PR}]—NHR^{PR}, —O—C(O)—CH[(CH₂)₂C(O)NHR^{PR}]—NHR^{PR}, —O—C(O)—CH(CH₂C(O)NHR^{PR})—NHR^{PR}, —O—C(O)—CHR⁴²—NHR^{PR}, —NH—(CH₂)₁₋₄—C(O)OR⁴⁶ or —O—C(O)—(CH₂)₁₋₄—NHR⁴⁷ where R⁴² is —H, —CH₃, —C₂H₅, —(CH₂)ₙ—C(O)—OR^{PR}, —CH₂—C(O)—OH, —CH₂—C(O)—NHR^{PR}, —CH₂F, —CH₂Cl, —CH₂Br, —CHOH—CH₃ or —CH₂OH, R⁴⁶ is —H, optionally substituted alkyl (e.g., —CH₃, —C₂H₅, —C₂H₃, —C₃H₇, —C₃H₅, —(CH₂)₁₋₈—NH₂, —(CH₂)₁₋₈—C(O)—OH, —(CH₂)₀₋₃—(CH═CH)₀₋₁—(CH₂)₀₋₃—CH₃, —(CH₂)₀₋₃—(CH═CH)₀₋₁—(CH₂)₀₋₃—CH₂F, —(CH₂)₀₋₃—(CH═CH)₀₋₁—(CH₂)₀₋₃—CH₂Br, —(CH₂)₀₋₃—(CH₂)₀₋₃C(O)—OH, —(CH₂)₀₋₃—(CH═CH)₀₋₁—(CH₂)₀₋₃—NH₂, —CF₃ or —C₂F₅) or a protecting group (e.g., t-butyl, phenyl, benzyl or substituted phenyl), R⁴⁷ is —H, optionally substituted alkyl (e.g., —CH₃, —C₂H₅, —C₂H₃, —C₃H₇, —C₃H₅, —(CH₂)₁₋₈—OH, —(CH₂)₁₋₈—C(O)—OH, —(CH₂)₀₋₃—(CH═CH)₀₋₁—(CH₂)₀₋₃—CH₃, —(CH₂)₀₋₃—(CH═CH)₀₋₁—(CH₂)₀₋₃—CH₂F, —(CH₂)₀₋₃—(CH═CH)₀₋₁—(CH₂)₀₋₃—CH₂Br, —(CH₂)₀₋₃—(CH═CH)₀₋₁—(CH₂)₀₋₃—C(O)—OH, —(CH₂)₀₋₃—(CH═CH)₀₋₁—(CH₂)₀₋₃—NH₂, —CF₃ or —C₂F₅) or a protecting group (e.g., t-butyl, phenyl, benzyl or substituted phenyl) and R^{PR} is —H or an independently selected protecting group such as C1-C8 optionally substituted alkyl and n is 0, 1, 2, or 3, or

optionally substituted heterocycle, —O—[C(O)]ₘ—(CH₂)ₙ-optionally substituted heterocycle, —(CH₂)ₙ-optionally substituted heterocycle or optionally substituted cycloalkyl, where the heterocycle is C-linked or N-linked, e.g., 2-pyridinyl, N-pyridinyl, 3-pyridinyl, 4-pyridinyl, 2-pyridyl, 3-pyridyl, 4-pyridyl, 5-pyridyl, 6-pyridyl, 1-pyrimidinyl, 2- pyrimidinyl, 4-pyrimidinyl, 5-pyrimidinyl, 6-pyrimidinyl, 3-pyridazinyl, 4-pyridazinyl, 2-pyrazinyl, 3-pyrazinyl, 5-pyrazinyl, 6-pyrazinyl, N-pyrrolyl, 2-pyrrolyl, 3-pyrrolyl, 2-imidazolyl, 4-imidazolyl, 5-imidazolyl, N-imidazolyl, 3-pyrazolyl, 4-pyrazolyl, 3-pyridazinyl, 4-pyridazinyl, 5-pyridazinyl, 6-pyridazinyl, 3-isothiazolyl, 4-isothiazolyl, 5-isothiazolyl, 3-isoxazolyl, 4-isoxazolyli 5-isoxazolyl, 2-thiazolyl, 4-thiazolyl, 5-thiazolyl, 2-oxazolyl, 3-oxazolyl, 4-oxazolyl, 5-oxazolyl, 1,2,4-oxadiazol-5-yl, 1,2,4-triazol-3-yl, 1,2,3-triazol-4-yl, tetrazol-5-yl, benzimidazol-2-yl, indol-3-yl, 1 H-indazol-3-yl, 1 H-pyrrolo[2,3-b]pyrazin-2-yl, 1H-pyrrolo[2,3-b]pyridin-6yl, 1H-imidazo[4,5-b]pyridin-2-yl, 1H-imidazo[4,5-c]pyridin-2-yl, 1H-imidazo[4,5-b]pyrazin-2-yl, benzopyranyl, furyl, thienyl, pyrrolyl, oxazolyl, isoxazolyl, thiazolyl, isothiazolyl, imidazolyl, pyrazolyl, 1,2,3-oxadiazolyl, 1,2,4,-oxadiazolyl, 1,3,4-oxadiazolyl, furazanyl, 1 ,2,3-thiadiazolyl, 1,2,4-thiadiazolyl, 1,3,4-thiadiazolyl, 1,2,3-triazolyl, 1,2,4-triazolyl, 1-isoquinolyl, 4-isoquinolyl, 2-quinazolinyl, 1-methyl-2-indolyl, tetrazolyl, pyridyl, pyridazinyl, pyrimidinyl, pyrazinyl, triazinyl, benzofuranyl, isobenzofuranyl, benzo[b]thienyl, indolyl, isoindolyl, 1H-indazolyl, benzimidazolyl, benzoxazolyl, 1,2-benzoisoxazolyl, benzothiazolyl, 1,2-benzoisothiazolyl, 1H-benzotriazolyl, 2-silabenzenyl, 3-silabenzenyl, 4-silabenzenyl, 5-silabenzenyl, quinolyl, isoquinolyl, cinnolinyl, quinazolinyl, quinoxalinyl, phthalazinyl, naphthylidinyl, purinyl, pteridinyl, carbazolyl, α-carbolinyl, β-carbolinyl, γ-carbolinyl, acridinyl, phenoxazinyl, phenothiazinyl, phenazinyl, phenoxathiinyl, thianthrenyl, phenathridinyl, phenathrolinyl, indolizinyl, pyrrolo[1,2-b]pyridazinyl, pyrazolo[1,5-a]pyridyl, imidazo[1,2-a]pyridyl, imidazo[1,5-a]pyridyl, imidazo[1,2-b]pyridazinyl, imidazo[1,2-a]pyrimidinyl, 1,2,4-triazolo[4,3-a]pyridyl, 1,2,4-triazolo[4,3-b]pyridazinyl, 2-benzothiazolyl, 2-benzoxazolyl, 2-benzimidazolyl, 3-isoxazolyl, 5-isoxazolyl, 5-methyl-3-isoxazolyl, 5-phenyl-3-isoxazolyl, 4-thiazolyl, 3-methyl-2-pyrazinyl, 5-methyl-2-pyrazinyl, 6-methyl-2-pyrazinyl, 5-chloro-2-thienyl, 3-furyl, benzofuran-2-yl, benzothien-2-yl, 2H-1-benzopyran-3-yl, 2,3-dihydrobenzopyran-5-yl, 1-methylimidazol-2-yl, q uinoxalin-2-yl, piperon-5-yl, 4,7-dichlorobenzoxazol-2-yl, 4,6-dimethyl-pyrimidin-2-yl, 4-methylpyrimidin-2-yl, 2,4-dimethylpyrimidin-6-yl, 2-methylpyrimidin-4-yl, 4-methylpyrimidin-6-yl, 6-chloropiperon-5-yl, 5-chloroimidazo[1,2-a]pyridin-2-yl, 1-H-inden-3-yl, 1-H-2-methyl-inden-2-yl, 3,4-dihydronaphth-1-yl, S-4-isopropenylcyclohexen-1-yl, 4-dihydronaphth-2-yl, 3-hydroxy-5-hydroxymethyl-2-methyl-4-pyridinyl, 5-methoxycarbonyl-2-furanyl, cycloheptyl, cyclohexyl, cyclopentyl, cyclooxyl, cyclobutyl, cyclobutenyl, 5-chloro-2-hydroxyphenyl, 5-chloro-2-methoxyphenyl, 2-methanesulfonylaminophenyl, 3-aminophenyl, 2-methoxyphenyl, 5-ethyl-2-furanyl, 3-methoxyphenyl, 2-aminophenyl, 2-furanyl, 3,5-dimethyl-4-hydroxyphenyl, 5-acetyloxymethyl-2-furanyl, 5-(4-carboxyphenyl)-2-furanyl, 5-(4-methanesulfonylphenyl)-2-furanyl, 5-(3,4-dimethoxyphenyl)-2-furanyl, 5-(4-methanesulfonylaminophenyl)-2-furanyl, 5-(4-bromophenyl)-2-oxazolyl, 5-(4-methoxyphenyl)-2-furanyl, 5-(1-cyclohexen-1-yl)-2-furanyl, 5-cyclohexyl-2-furanyl, 5-(3-trifluoromethylphenyl-2-furanyl, 5-(4-methylphenyl)-2-furanyl, 2-(4-chlorophenyl)-3-furanyl, 5-(4-chlorophenyl)-2-furanyl, 5-(4-fluorophenyl)-2-furanyl, 2-benzyloxy-5-chlorophenyl, 4-benzyloxyphenyl, 3-(4-t-butylphenyloxy)phenyl, 3-benzoyl-2,4-dichlorophenyl, 2-chloro-3-benzyloxyphenyl, 3-(4-chlorophenoxyl) phenyl, 1H-indol-3-yl, 2-fluorenyl, 2-naphthyl, 2-hydroxy-1-naphthyl, 2-quinolinyl, 5-chloro-2-benzofuranyl, 1-aziridinyl, 2-aziridinyl, N-pyrrolidinyl, 1-pyrrolidinyl, 2-pyrrolidinyl, 1-aziridyl, 1-azetedyl, 1-pyrrolyl, 1-imidazolyl, 1-pyrazolyl, 1-piperidinyl, 3-oxathiolanyl, 4-oxathiolanyl, 5-oxathiolanyl, N-2H-1,5,2-dithiazinyl, 3-2H-1,5,2-dithiazinyl, 4-2H-1,5,2-dithiazinyl, 6-2H-1,5,2-dithiazinyl, 1-cyclohexenyl, 2-cyclohexenyl, 3-cyclohexenyl, 4-cyclohexenyl, 5-cyclohexenyl, 1-cyclopentenyl, 2-cyclopentenyl, 3-cyclopentenyl, 1,3-cyclopentadienyl, 1-cycloheptenyl, 1,3-cycloheptadienyl, isothiazolyl, isoxazolyl, oxiranyl, azetidinyl, oxetanyl, thietanyl, pyrrolidinyl, tetrahydrofuryl, thiolanyl, piperidyl, tetrahydropyranyl, morpholinyl, thiomorpholinyl, piperazinyl, pyrrolidino, piperidino, N-morpholino, morpholino or thiomorpholino, any of which optionally has 1, 2, 3 or 4 independently selected substitutions as described herein, e.g., —OH, —OR^{PR}, ═O, —SH, —SR^{PR}, ═S, —F, —Cl, —Br, —I, —C(O)OR^{PR}, —C(O)SRPR, —C(O)OCH₃, —C(O)O—C1-8 optionally substituted alkyl, C1-8 optionally substituted alkyl, C1-8 ether, C1-8 thioether, C1-8 ester, C1-8 thioester, —CN, —SCN, —NO₂, —N₃, —NH₂, —NHR^{PR}, —NH—C1-8 optionally substituted alkyl, —N(C1-8 optionally substituted alkyl)₂, where each optionally substituted alkyl is independently selected, C1-8 haloalkyl, C1-8 hydroxyalkyl, C1-8 aralkyl, C1-8 alkenyl, C1-8 alkoxy, C1-8 haloalkyloxy, C1-8 alkylthio, C1-8 cycloalkyl, C1-8 cycloalkylalkyl, C1-8 cycloalkyloxy C1-8 alkylsulfonyl, C1-8 sulfamoyl, C1-8 alkanoyl, C1-8 alkoxycarbonyl or another substituent described herein, where R^{PR} independently are —H or a protecting group, m is 0 or 1 and n is 0, 1, 2 or 3, e.g., m and n are both 0, m is 1 and n is 0, m is 0 and n is 1 or m and n are both 1, and where exemplary substitutions include a halogen such as —F or —Cl at the 1-, 2-, 3-, 4- or 5- position of any of these moieties, an ester or hydroxyl at the 1-, 2-, 3-, 4- or 5-position of any of these moieties, an ether or thioether at the 1-, 2-, 3-, 4- or 5- position of any of these moieties and/or optionally substituted alkyl at the 1-, 2-, 3-, 4- or 5- position of any of these moieties, where any such substitution is compatible with the chemical structure and/or nomenclature of the cyclic moiety, e.g., cyclobutyl moieties can not be substituted at the 5-position and ring oxygen atoms can not be substituted, or

carboxyl which is optionally substituted, e.g., —C(O)OH, —C(O)OR^{PR}, —C(O)OM, —C(O)O—CH₃, —C(O)—O—(CH₂)ₙ—CH₃, —C(O)—O—CH(CH₃)—(CH₂)ₙ—CH₃, —C(O)—O—C(CH₃)₂—(CH₂)ₙ—CH₃, —C(O)—O—(CH₂)ₙ—C(O)OR^{PR}, —C(O)—O—CH(CH³)—(CH²)ₙ—C(O)OR^{PR}, —C(O)—O—C(CH₃)₂—(CH₂)ₙ—C(O)OR^{PR}, —C(O)—O—(CH₂)ₙ—CH₂OR^{PR}, —C(O)—O—CH(CH₃)—(CH₂)ₙ—CH₂OR^{PR}, —C(O)—O—C(CH₃)₂—(CH₂)ₙ—CH₂OR^{PR}, —C(O)—O—(CH₂)ₙ—CH₂NHR^{PR}, —C(O)—O—CH(CH₃)—(CH₂)ₙ—CH₂NHR^{PR}, —C(O)—O—C(CH₃)₂—(CH₂)ₙ—CH₂NHR^{PR}, —C(O)—O—(CH₂)ₙ—CH₂SR^{PR}, —C(O)—O—CH(CH₃)—(CH₂)ₙ—CH₂SR^{PR}, —C(O)—O—C(CH₃)₂—(CH₂)ₙ—CH₂SR^{PR}, —C(O)O-organic moiety, —C(O)O—(CH₂)ₙ-optionally substituted phenyl or —C(O)O—(CH₂)ₙ-optionally substituted alkyl, wherein the phenyl or alkyl moieties are optionally substituted with 1, 2 or 3 independently selected with substituents described herein, e.g., —F, —Cl, —Br, —I, —OH, —CH₃, —C₂H₅, —O—CH₃, —O—C₂H₅, —NO₂, —CN, —SCN, —NH₂, —C(O)OR^{PR} or —(CH₂)₁₋₄—C(O)—OR^{PR}, where n is 0, 1, 2, 3, 4, 5 or 6, R^{PR} is —H or a protecting group such as methyl, ethyl, propyl or butyl, and M is a metal such as an alkali metal, e.g., Li⁺, Na⁺ or K⁺ or M is another counter ion such as an ammonium ion, or

carbonate, e.g., —O—C(O)—O—CH₃, —O—C(O)—O—(CH₂)ₙ—CH₃, —O—C(O)—O—CH(CH₃)—(CH₂)ₙ—CH₃, —O—C(O)—O—CH₂-halogen, —O—C(O)—O—(CH₂)ₙ—CH₂-halogen, —O—C(O)—O—CH(CH₃)—(CH₂)ₙ—CH₂-halogen, —O—C(O)—O—C(CH₃)₂—(CH₂)ₙ—CH₃, —O—C(O)—O—(CH₂)ₙ—C(O)OR^{PR}, —O—C(O)—O—CH(CH₃)—(CH₂)ₙ—C(O)OR^{PR}, —O—C—(O)—O—C(CH₃)₂—(CH₂)ₙ—C(O)OR^{PR}, —O—C(O)—O—CH(CH₃)—(CH₂)ₙ—C(O)OR^{PR}, —O—C(O)—O—C(CH₃)₂—(CH₂)ₙ—C(O)OR^{PR}, —O—C(O)—O—(CH₂)ₙ—CH₂OR^{PR}, —O—C(O)—O—CH(CH₃)—(CH₂)ₙ—CH₂OR^{PR}, —O—C(O)—O—C(CH₃)₂—(CH₂)ₙ—CH₂OR^{PR}, —O—C(O)—O—(CH₂)ₙ—CH₂NHR^{PR}, —O—C(O)—O—CH(CH₃)—(CH₂)ₙ—CH₂NHR^{PR}, —O—C(O)—O—C(CH₃)₂—(CH₂)ₙ—CH₂NHR^{PR}, —O—C(O)—O—(CH₂)ₙ—CH₂SR^{PR}, —O—C(O)—O—CH(CH₃)—(CH₂)ₙ—CH₂SR^{PR}, —O—C(O)—O—C(CH₃)₂—(CH₂)ₙ—CH₂SR^{PR}, —O—C(O)—O—organic moiety, —O—C(O)—O(CH₂)ₙ-optionally substituted phenyl or —C(O)—O—(CH₂)ₙ-optionally substituted alkyl, wherein the phenyl or alkyl moieties are optionally substituted with 1, 2 or 3, 4 or more independently selected with substituents described herein, e.g., —F, —Cl, —Br, —I, —OH, —CH₃, —C₂H₅, —O—CH₃, —O—C₂H₅, —NO₂, —CN, —SCN, —NH₂, —C(O)OR^{PR} or —(CH₂)₁₋₄—C(O)—OR^{PR}, and wherein n is 0, 1, 2, 3, 4, 5 or 6 and R^{PR} is —H or a protecting group, or

carbamate, e.g., —O—C(O)—NH₂, —O—C(O)—NH—CH₃, —O—C(O)—NH—C₂H₅, —O—C(O)—NH—C₃H₇, —O—C(O)—NH—C₄H₉, —O—C(O)—NH—C₂H₃, —O—C(O)—NH—C₃H₅, —O—C(O)—NH—C₄H₇, —O—C(O)—NHR^{PR}, —O—C(O)—N[(CH₂)ₙCH₃]—CH₃, —O—C(O)—N[(CH₂)ₙCH₃]—C₂H₅, —O—C(O)—N[(CH₂)ₙCH₃]—C₃H₇, —O—C(O)—N[(CH₂)ₙCH₃]—C₄H₉, —O—C(O)—N[(CH₂)ₙCH₃, —O—C(O)—N[(CH₂)ₙCH₃]—C₃H₅, —O—C(O)—N[(CH₂)ₙCH₃]—C₄H₇, —O—C(O)—NH-organic moiety, —O—C(O)—NR⁴⁸-organic moiety, —NH—C(O)—O-organic moiety, —NR48—C(O)—O-organic moiety, wherein the organic moiety is as described herein, e.g., it optionally comprises about 1-20 carbon atoms, and wherein R⁴⁸ is —H, a protecting group, an organic moiety or R⁴⁸ together with the organic moiety is a protecting group and the organic moiety optionally is optionally substituted alkyl such as i-propyl, n-propyl, t-butyl, n-butyl, n-hexyl, n-octyl, n-decyl, —(CH₂)ₘ—OH, —(CH₂)ₘ—F, —(CH₂)ₘ—Cl, —(CH₂)ₘ—Br, —(CH₂)ₘ—NH₂, —(CH₂)ₘ—C(O)—OH, —(CH₂)ₘ—C(O)—H, —(CH₂)ₘ—C(O)—CH₃, —(CH₂)ₙ—(O)ₚ—(CH₂)ₙ—CH₃, —(CH₂)ₙ—(O)ₚ—(CH₂)ₙ—CH₂OH, —(CH₂)ₙ—(CH═CH)ₚ—(CH₂)ₙ—CH₃, —(CH₂)ₙ—(CH═CH)ₚ—(CH₂)ₙ—CH₂OH, —(CH₂)ₙ—(CH═CH)ₚ—(CH₂)ₙ—CH₂F, —(CH₂)ₙ—(CH═CH)ₚ—(CH₂)ₙ—CH₂Br —(CH₂)ₙ—(CH═CH)ₚ—(CH₂)ₙ—C(O)—OR^{PR}, —(CH₂)ₙ—(CH═CH)ₚ—(CH₂)ₙ—NHR^{PR}, —(CH₂)ₙ—(C≡C)ₚ—(CH₂)ₙ—(C≡C)ₚ—(CH₂)ₙ—CH₂OH, —(CH₂)ₙ—(C≡C)ₚ—(CH₂)ₙ—CH₂F, —(CH₂)ₙ—(C≡C)ₚ—(CH₂)ₙ—CH₂Br, —(CH₂)ₙ—(C≡C)ₚ—(CH₂)ₙ—C(O)—OR^{PR}, —(CH₂)ₙ—(C≡C)ₙNHR^{PR}, —CF₃ or —C₂F₅, wherein R^{PR} is —H or a protecting group, m is 1, 2, 3, 4, 5 or 6, n independently are 0, 1, 2, 3, 4, 5 or 6 and p is 0 or 1, e.g., both n and p are 1 or p is 1 and both n are 2 or one n is 1, the other n is 2 and p is 1, or

phosphothioester or thiophosphate or an ether or thioether derivative thereof, e.g., —O—P(S)(OH)—OH, —O—P(S)(OH)—SH, —O—P(S)(OR^{PR})—OH, —O—P(S)(OR^{PR})—SH, —S—P(S)(OH)—OH, —O—P(S)(OH)—O—CH₃, —O—P(S)(OH)—O—C₂H₅, —O—P(S)(OH)—O—C₃H₇, —O—P(S)(OH)—O-optionally substituted alkyl, —S—P(S)(OH)—O-optionally substituted alkyl, —O—P(S)(OH)—S-optionally substituted alkyl, —O—P(S)(O-optionally substituted alkyl)-O-optionally substituted alkyl, —S—P(S)(O-optionally substituted alkyl)-O-optionally substituted alkyl, —O—P(S)(O-optionally substituted alkyl)-S-optionally substituted alkyl, where the optionally substituted alkyl moieties are as described herein and are independently selected, e.g., i-propyl, n-propyl, t-butyl, n-butyl, n-hexyl, n-octyl, n-decyl, —(CH₂)ₘ—OH, —(CH₂)ₘ—F, —(CH₂)ₘ—Cl, —(CH₂)ₘ—Br, —(CH₂)ₘ—NH₂, —(CH₂)ₘ—C(O)—OH, —(CH₂)ₘ—C(O)—H, —(CH₂)ₘ—C(O)—CH₃, —(CH₂)ₙ—(O)ₚ—(CH₂)ₙ—CH₃, —(CH₂)ₙ—(O)ₚ—(CH₂)ₙ—CH₂OH, (CH₂)ₙ—(CH═CH)ₚ—(CH₂)ₙ—CH₃, —(CH₂)ₙ—(CH═CH)ₚ—(CH₂)ₙ—CH₂OH, —(CH₂)ₙ—(CH═CH)ₚ—(CH₂)ₙ—CH₂F, —(CH₂)ₙ—(CH═CH)ₚ—(CH₂)ₙ—CH₂Br, —(CH₂)ₙ—(CH═CH)ₚ—(CH₂)ₙ—C(O)—OR^{PR}, —(CH₂)ₙ—(CH═CH)ₚ—(CH₂)ₙ—NHR^{PR}, —(CH₂)ₙ(C≡C)ₚ—(CH₂)ₙ—CH₃, —(CH₂)ₙ—(C≡C)ₚ—(CH₂)ₙ—CH₂OH, —(CH₂)ₙ—(C≡C)ₚ—(CH₂)ₙ—CH₂F, —(CH₂)ₙ—(C≡C)ₚ—(CH₂)ₙ—CH₂Br, —(CH₂)ₙ—(C≡C)ₚ—(CH₂)ₙ—C(O)—OR^{PR}, —(CH₂)ₙ(C≡C)ₚ—(CH₂)ₙ—NHR^{PR}, —CF₃ or —C₂F₅, wherein R^{PR} is —H or a protecting group, m is 1, 2, 3, 4, 5 or 6, n independently are 0, 1, 2, 3, 4, 5 or 6 and p is 0 or 1, e.g., both n and p are 1 or p is 1 and both n are 2 or one n is 1, the other n is 2 and p is 1, or

phosphonoester, phosphonate or an ether or thioether derivative thereof, e.g., —P(O)(OH)—OH, —P(O)(OH)—SH, —P(O)(OR^{PR})—OH, —P(O)(OR^{PR})—SH, —P(O)(OH)—OH, —P(O)(OH)—O—CH₃, —P(O)(OH)—O—C₂H₅, —P(O)(OH)—O—C₃H₇, —O—P(O)(OH)—H, —S—P(O)(OH)—H, —O—P(O)(OR^{PR})—H, —S—P(O)(OR^{PR})—H, —O—P(O)(OH)—CH₃, —O—P(O)(OH)—C₂H₅, —O—P(O)(OH)—C₃H₇, —O—P(O)(OH)-optionally substituted alkyl, —S—P(O)(OH)-optionally substituted alkyl, —P(O)(OH)—O-optionally substituted alkyl, —P(O)(OH)—S-optionally substituted alkyl, —P(O)(O-optionally substituted alkyl)-O-optionally substituted alkyl, —P(O)(O-optionally substituted alkyl)-S-optionally substituted alkyl, where the optionally substituted alkyl moieties are as described herein and are independently selected, e.g., i-propyl, n-propyl, t-butyl, n-butyl, n-hexyl, n-octyl, n-decyl, —(CH₂)ₘ—OH, —(CH₂)ₘ—F, —(CH₂)ₘ—Cl, —(CH₂)ₘ—Br, —(CH₂)ₘ—NH₂, —(CH₂)ₘ—C(O)—OH, —(CH₂)ₘ—C(O)—H, —(CH₂)ₘ—C(O)—CH₃, —(CH₂)ₙ—(O)ₚ—(CH₂)ₙ—CH₃, —(CH₂)ₙ—(O)ₚ—(CH₂)ₙ—CH₂OH, —(CH₂)ₙ—(CH═CH)ₚ—(CH₂)ₙ—CH₃, —(CH₂)ₙ—(CH═CH)ₚ—(CH₂)ₙ—CH₂OH, —(CH₂)ₙ—(CH═CH)ₚ—(CH₂)ₙ—CH₂F, —(CH₂)ₙ—(CH═CH)ₚ—(CH₂)ₙ—CH₂Br, —(CH₂)—(CH═CH)ₚ—(CH₂)ₙ—C(O)—OR^{PR}—(CH₂)ₙ—(CH═CH)ₚ—(CH₂)ₙ—NHR^{PR}, —(CH₂)ₙ—(C≡C)ₚ—(CH₂)ₙ—CH₃, —(CH₂)ₙ—(C≡C)ₚ—(CH₂)ₙ—CH₂OH, —(CH₂)ₙ—(C≡C)ₚ—(CH₂)ₙ—CH₂F, —(CH₂)ₙ—(C≡C)ₚ—(CH₂)ₙ—CH₂Br, —(CH₂)—(C≡C)ₚ—(CH₂)ₙ—C(O)—OR^{PR}, —(CH₂)ₙ—(C≡C)ₚ—(CH₂)ₙ—NHR^{PR}, —CF₃ or —C₂F₅, wherein R^{PR} is —H or a protecting group, m is 1, 2, 3, 4, 5 or 6, n independently are 0, 1, 2, 3, 4, 5 or 6 and p is 0 or 1, e.g., both n and p are 1 or p is 1 and both n are 2 or one n is 1, the other n is 2 and p is 1, or

sulfate, sulfate ester or an ether or thioether derivative thereof, e.g., —O—S(O)(O)—OH, —O—S(O)(O)—SH, —O—S(O)(O)—OR^{PR}, —O—S(O)(O)—O—CH₃, —O—S(O)(O)—O—C₂H₅, —O—S(O)(O)—O—C₃H₇, —O—S(O)(O)—S—CH₃, —O—S—(O)(O)—O—CH₂—CH(O—C(O)—(CH2)_{y}(CH═CH)_{q}(CH₂)_{y}—CH₃)—CH₂—O—C(O)—(CH₂)_{y}(CH═CH)_{q}(CH₂)_{y}—CH₃, O—S—(O)(O)—O—CH₂—CH(O—C(O)—(CH₂)ₓCH₃)—CH₂—O—C(O)—(CH₂)ₓCH₃), —O—S—(O)(O)—O—CH₂—CH(O—C(O)—(CH₂)₁₄CH₃)—CH₂—O—C(O)—(CH₂)₁₄CH₃), —O—S—(O)(O)—O—CH₂—CH(O—C(O)—(CH₂)₁₂CH₃)—CH₂—O—C(O)—(CH₂)₁₂CH₃), —O—S(O)(O)—O-optionally substituted alkyl, —O—S(O)(OH)—S-optionally optionally substituted alkyl, where the optionally substituted alkyl moiety is as described herein, e.g., i-propyl, n-propyl, t-butyl, n-butyl, n-hexyl, n-octyl, n-decyl, —(CH₂)ₘ—OH, —(CH₂)ₘ—F, —(CH₂)ₘ—Cl, —(CH₂)ₘ—Br, —(CH₂)ₘ—NH₂, —(CH₂)ₘ—C(O)-OH, —(CH₂)ₘ—C(O)—H, —(CH₂)ₘ—C(O)—CH₃, —(CH₂)ₙ—(O)ₚ—(CH₂)ₙ—CH₃, —(CH₂)ₙ—(O)ₚ—(CH₂)ₙ—CH₂OH, —(CH2)ₙ—(CH═CH)ₚ—(CH₂)ₙ—CH₃, —(CH₂)ₙ—(CH═CH)ₚ—(CH₂)ₙ—CH₂OH, —(CH₂)ₙ—(CH═CH)ₚ—(CH₂)ₙ—CH₂F, —(CH₂)ₙ—(CH═CH)ₚ—(CH₂)ₙ—CH₂Br, —(CH₂)ₙ—(CH═CH)ₚ—(CH₂)ₙ—C(O)—OR^{PR}, —(CH₂)ₙ—(CH═CH)ₚ—(CH₂)ₙ—NHR^{PR}, —(CH₂)ₙ—(C≡C)ₚ—(CH₂)ₙ—CH₃, —(CH₂)ₙ—(C≡C)ₚ—(CH₂)ₙ—CH₂OH, —(CH₂)ₙ—(C≡C)ₚ—(CH₂)ₙ—CH₂F, —(CH₂)ₙ—(C≡C)ₚ—(CH₂)ₙ—CH₂Br, —(CH₂)ₙ—(C≡C)ₚ—(CH₂)ₙ—C(O)—OR^{PR}, —(CH₂)ₙ—(C≡C)ₚ—(CH₂)ₙ—NHR^{PR}, —CF₃ or —C₂F₅, wherein R^{PR} is —H or a protecting group, m is 1, 2, 3, 4, 5 or 6, n independently are 0, 1, 2, 3, 4, 5 or 6, p is 0 or 1, q is 0 or 1, x independently are 0, 1, 2, 3, 4, 5, 6, 7, 8, 9, 10, 11, 12, 13, 14, 15, 16 or 17, y independently are 0, 1, 2, 3, 4, 5, 6, 7, 8 or 9 and substituents bonded at double bonds are in the cis, trans or mixed cis and trans configuration, wherein In some embodiments, both n and p are 1 or p is 1 and both n are 2 or one n is 1, the other n is 2 and p is 1, or

optionally substituted oxime, e.g., ═NOH, ═NOCH₃, ═NOC₂H₅, ═NOC₃H₇, ═N—(CH₂)ₙ—(X)_{q}—(CH₂)ₙ-optionally substituted alkyl, where X is —O—, —C(O)—, —S— or —NH— and the optionally substituted alkyl moiety is as described herein, e.g., i-propyl, n-propyl, t-butyl, n-butyl, n-hexyl, n-octyl, n-decyl, —(CH₂)ₘ—OH, —(CH₂)ₘ—F, —(CH₂)ₘ—Cl, —(CH₂)ₘ—Br, —(CH₂)ₘ—NH₂, —(CH₂)ₘ—C(O)—OH, —(CH₂)ₘ—C(O)—H, —(CH₂)ₘ—C(O)—CH₃, —(CH₂)ₘ-heterocycle, —(CH₂)ₙ—(O)ₚ—(CH₂)ₙ—CH₃, —(CH₂)ₙ—(O)ₚ—(CH₂)ₙ—CH₂OH, —(CH₂)ₙ—(CH═CH)ₚ—(CH₂)ₙ—CH₃, —(CH₂)ₙ—(CH═CH)ₚ—(CH₂)ₙ—CH₂OH, —(CH₂)ₙ—(CH═CH)ₚ—(CH₂)ₙ—CH₂F, —(CH₂)ₙ—(CH═CH)ₚ—(CH₂)ₙ—CH₂Br, —(CH₂)ₙ—(CH═CH)ₚ—(CH₂)ₙ—C(O)—OR^{PR}, —(CH₂)ₙ—(CH═CH)ₚ—(CH₂)ₙ—NHR^{PR}, —(CH₂)ₙ—(C≡C)ₚ—(CH₂)ₙ—CH₃, —(CH₂)ₙ—(C≡C)ₚ—(CH₂)ₙ—CH₂OH, —(CH₂)ₙ—(C≡C)ₚ—(CH₂)ₙ—CH₂F, —(CH₂)ₙ—(C≡C)ₚ—(CH₂)ₙ—CH₂Br, —(CH₂)ₙ—(C≡C)ₚ—(CH₂)ₙ—C(O)—OR^{PR}, —(CH₂)ₙ—(C≡C)ₚ—(CH₂)ₙ—NHR^{PR}, —CF₃ or —C₂F₅, wherein R^{PR} is —H or a protecting group, m is 1, 2, 3, 4, 5 or 6, n independently are 0, 1, 2, 3, 4, 5 or 6, p is 0 or 1, and q is 0 or 1, e.g., both n and p are 1 or p is 1 and both n are 2 or one n is 1, the other n is 2 and p is 1, or

sulfite, sulfite ester, sulfite ether, sulfite or sulfoxide, e.g., —O—S(O)—OH, —O—S(O)—OR^{PR}, —O—S(O)—O—CH₃, —O—S(O)—O—C₂H₅, —O—S(O)—O—C₃H₇, —O—S(O)—O-organic moiety, —O—S(O)—O-optionally substituted alkyl, —S(O)—O—CH₃, —S(O)—O—C₂H₅, —S(O)—O—C₃H₇, —S(O)-organic moiety, —S(O)-optionally substituted alkyl, where the optionally substituted alkyl moiety is as described herein, e.g., i-propyl, n-propyl, t-butyl, n-butyl, n-hexyl, n-octyl, n-decyl, —(CH₂)ₘ—OH, —(CH₂)ₘ—F, —(CH₂)ₘ—Cl, —(CH₂)ₘ—Br, —(CH₂)ₘ—NH₂, —(CH₂)ₘ—C(O)—OH, —(CH₂)ₘ—C(O)—H, —(CH₂)ₘ—C(O)—CH₃, —(CH₂)ₙ—(O)ₚ—(CH₂)ₙ—CH₃, —(CH₂)ₙ—(O)ₚ—(CH₂)ₙ—CH₂OH, —(CH₂)ₙ—(CH═CH)ₚ—(CH₂)ₙ—CH₃, —(CH₂)ₙ—(CH═CH)ₚ—(CH₂)ₙ—CH₂OH, —(CH₂)ₙ—(CH═CH)ₚ—(CH₂)ₙ—CH₂F, —(CH₂)ₙ—(CH═CH)ₚ—(CH₂)ₙ—CH₂Br, —(CH₂)ₙ—(CH═CH)ₚ—(CH₂)ₙ—C(O)—OR^{PR}, —(CH₂)ₙ—(CH═CH)ₚ—(CH₂)ₙ—NHR^{PR}, —(CH₂)ₙ—(C≡C)ₚ—(CH₂)ₙ—CH₃, —(CH₂)ₙ—(C≡C)ₚ—(CH₂)ₙ—CH₂OH, —(CH₂)ₙ—(C≡C)ₚ—(CH₂)ₙ—CH₂F, —(CH₂)ₙ—(C≡C)ₚ—(CH₂)ₙ—CH₂Br, —(CH₂)ₙ—(C≡C)ₚ—(CH₂)ₙ—C(O)—OR^{PR}, —(CH₂)ₙ—(C≡C)ₚ—(CH₂)ₙ—NHR^{PR}, —CF₃ or —C₂F₅, wherein R^{PR} is —H or a protecting group, m is 1, 2, 3, 4, 5 or 6, n independently are 0, 1, 2, 3, 4, 5 or 6 and p is 0 or 1, e.g., both n and p are 1 or p is 1 and both n are 2 or one n is 1, the other n is 2 and p is 1, and the organic moiety is as described herein, or

sulfonamide or a sulfonamide derivative, e.g., —S(O)(O)—NH₂, —S(O)(O)—NHR^{PR}, —S(O)(O)—NH-optionally substituted alkyl, —NH—S(O)(O)-optionally substituted alkyl, —S(O)(O)—NH—CH₃, —S(O)(O)—NH—C₂H₅, —S(O)(O)—NH—C₃H₇, —NH—S(O)(O)—CH₃, —NH—S(O)(O)—C₂H₅, —NH—S(O)(O)—C₃H₇, where the optionally substituted alkyl moiety is as described herein, e.g., i-propyl, n-propyl, t-butyl, n-butyl, n-hexyl, n-octyl, n-decyl, —(CH₂)ₘ—OH, —(CH₂)ₘ—F, —(CH₂)ₘ—Cl, —(CH₂)ₘ—Br, —(CH₂)ₘ—NH₂, —(CH₂)ₘ—C(O)—OH, —(CH₂)ₘ—C(O)—H, —(CH₂)ₘ—C(O)—CH₃, —(CH₂)ₙ—(O)ₚ—(CH₂)ₙ—CH₃, —(CH₂)ₙ—(O)ₚ—(CH₂)ₙ—CH₂OH, —(CH₂)ₙ—(CH═CH)ₚ—(CH₂)ₙ—CH₃, —(CH₂)ₙ—(CH═CH)ₚ—(CH₂)ₙ—CH₂OH, —(CH₂)ₙ—(CH═CH)ₚ—(CH₂)ₙ—CH₂F, —(CH₂)ₙ—(CH═CH)ₚ—(CH₂)ₙ—CH₂Br, —(CH₂)—(CH═CH)ₚ—(CH₂)—C(O)—OR^{PR}, —(CH₂)ₙ—(CH═CH)ₚ—(CH₂)ₙ—NHR^{PR}, —(CH₂)ₙ—(C≡C)ₚ—(CH₂)ₙ—CH₃, —(CH₂)ₙ—(C≡C)ₚ—(CH₂)ₙ—CH₂OH, —(CH₂)ₙ—(C≡C)ₚ—(CH₂)ₙ—CH₂F, —(CH₂)ₙ—(C≡C)ₚ—(CH₂)ₙ—CH₂Br, —(CH₂)ₙ—(C≡C)ₚ—(CH₂)ₙ—C(O)—OR^{PR}, —(CH₂)ₙ—(C≡C)ₚ—(CH₂)—NHR^{PR}, —CF₃ or —C₂F₅, wherein R^{PR} is —H or a protecting group, m is 1, 2, 3, 4, 5 or 6, n independently are 0, 1, 2, 3, 4, 5 or 6 and p is 0 or 1, e.g., both n and p are 1 or p is 1 and both n are 2 or one n is 1, the other n is 2 and p is 1, or

sulfamate or a sulfamate derivative, e.g., —O—S(O)(O)—NH₂, —O—S(O)(O)—NHR^{PR}, —O—S(O)(O)—N(RD)₂, —O—S(O)(O)—NH-optionally substituted alkyl, —NH—S(O)(O)—O-optionally substituted alkyl, —O—S(O)(O)—NH—C(O)—CH₃, —O—S(O)(O)—NH—C(O)-optionally substituted alkyl, —O—S(O)(O)—NH—CH₃, —O—S(O)(O)—NH—C₂H₅, —O—S(O)(O)—NH—C₃H₇, —O—S(O)(O)—N(C(O)-optionally substituted alkyl)-R⁵², —O—S(O)(O)—N(C(O)—N-optionally substituted alkyl)-R⁵², —NH—S(O)(O)—O—CH₃, —NH—S(O)(O)—O—C₂H₅, —NH—S(O)(O)—O—C₃H₇, —NH—S(O)(O)—O-optionally substituted alkyl, where any optionally substituted alkyl moiety is as described herein, e.g., i-propyl, n-propyl, t-butyl, n-butyl, n-hexyl, n-octyl, n-decyl, —(CH₂)ₘ—OH, —(CH₂)ₘ—F, —(CH₂)ₘ—Cl, —(CH₂)ₘ—Br, —(CH₂)ₘ—NH₂, —(CH₂)ₘ—C(O)—OH, —(CH₂)ₘ—C(O)—H, —(CH₂)ₘ—C(O)—CH₃, —(CH₂)ₙ—(O)ₚ—(CH₂)ₙ—CH₃, —(CH₂)ₙ—(O)ₚ—(CH₂)ₙ—CH₂OH, —(CH₂)ₙ—(CH═CH)ₚ—(CH₂)ₙ—CH₃, —(CH₂)ₙ—(CH═CH)ₚ—(CH₂)ₙ—CH₂OH, —(CH₂)ₙ—(CH═CH)ₚ—(CH₂)ₙ—CH₂F, —(CH₂)ₙ—(CH═CH)ₚ—(CH₂)ₙ—CH₂Br, —(CH₂)ₙ—(CH═CH)ₚ—(CH₂)—C(O)—OR^{PR}, —(CH₂)ₙ—(CH═CH)ₚ—(CH₂)ₙ—NHR^{PR}, —(CH₂)ₙ—(C≡C)ₚ—(CH₂)ₙ—CH₃, —(CH₂)ₙ—(C≡C)ₚ—(CH₂)ₙ—CH₂OH, —(CH₂)ₙ—(C≡C)ₚ—(CH₂)ₙ—CH₂F, —(CH₂)ₙ—(C≡C)ₚ—(CH₂)ₙ—CH₂Br, —(CH₂)ₙ—(C≡C)ₚ—(CH₂)ₙ—C(O)—OR^{PR}, —(CH₂)ₙ—(C≡C)ₚ—(CH₂)ₙ—NHR^{PR}, —CF₃ or —C₂F₅, wherein R^{PR} is —H or a protecting group, RD independently are —H, optionally substituted alkyl (e.g., —CH₃, —C₂H₅, —C₃H₇, —CHO, —CH₂OH), acyl, benzoyl or benzyl, R⁵² is —H, optionally substituted alkyl, —COOH, —COOR^{PR}, —COO-optionally substituted alkyl or —C(O)—N(R⁵³)₂, R⁵³ independently are —H, optionally substituted alkyl, optionally substituted aryl, optionally substituted alkylaryl or optionally substituted arylalkyl, or both R⁵³ together with the nitrogen atom to which they are bonded are an N-containing ring such as morpholino or a C2-C6 polyemthyleneimino residue, m is 1, 2, 3, 4, 5 or 6, n independently are 0, 1, 2, 3, 4, 5 or 6 and p is 0 or 1, e.g., both n and p are 1 or p is 1 and both n are 2 or one n is 1, the other n is 2 and p is 1, or

a sulfonate, a sulfamide, a sulfinamide or a sulfurous diamide, e.g., —O—S(O)(O)—CH₂-optionally substituted alkyl, —O—S(O)(O)-optionally substituted alkyl, —NH—S(O)(O)—NHR^{PR}, —NH—S(O)(O)—NH-optionally substituted alkyl, —NH—S(O)—NHR^{PR}, —NH—S(O)—NH-opyionally substituted alkyl, —S(O)—NHR^{PR}, —S(O)—NHCH₃, —S(O)—N(CH₃)₂, —S(O)—NHC₂H₅, —S(O)—NH-optionally substituted alkyl, —NH—S(C)—NHR^{PR}, —NH—S(O)—NHCH₃, —NH—S(O)—NHC₂H₅ or —NH—S(O)—NH-optionally substituted alkyl, or

a monosaccharide, e.g., a D-, L- or DL-mixture of glucose, fructose, mannose, idose, galactose, allose, gulose, altrose, talose, fucose, erythrose, threose, lyxose, erythrulose, ribulose, xylulose, ribose, arabinose, xylose, psicose, sorbose, tagatose, glyceraldehyde, dihydroxyacetone, a monodeoxy derivative of these monosaccharides such as rhamnose, glucuronic acid or a salt of glucuronic acid, any of which are unprotected, partially protected (e.g., less than all hydroxyl groups are protected) or fully protected with independently selected protecting groups (e.g., acetoxy or propionoxy), or

an oligosaccharide, e.g., 2, 3, 4 or more linked and independently selected monosaccharides that comprise a D-, L- or DL-mixture of glucose, fructose, mannose, idose, galactose, allose, gulose, altrose, talose, fucose, erythrose, threose, lyxose, erythrulose, ribulose, xylulose, ribose, arabinose, xylose, psicose, sorbose, tagatose, glyceraldehyde, N-acetylglucosamine, dihydroxyacetone or a monodeoxy or dideoxy derivative of any of these, with adjacent monosaccharides having the glycosidic linkage at the anomeric carbon of each monosaccharide unit independently alpha or beta linked, e.g., 1→2, 1→3, 1→4, and/or 1→6 glycosidic bonds in the α- and/or β-configuration, e.g., -glucose-mannose, -glucose-mannose-mannose, -mannose-mannose, -mannose-mannose-mannose, -glucose-galactose, -galactose-glucose, -fructose-galactose, -galactose-fructose, -galactose-galactose, -galactose-mannose, -glucuronic acid-glucose, -glucose-glucose, —(O-1β)-D-glucopyranosyl-(1α-O-4)-D-glucopyranoside, —(O-1β)-tetra-O-acetyl-D-glucopyranosyl-(1α-O-4)-tri-O-acetyl-D-glucopyranoside, —(O-1β)-D-galactopyranosyl-(1β-O-4)-D-glucopyranoside, wherein one or more of the monosaccharides are optionally partially or fully protected, e.g., with —C(O)—CH₃ or —C(O)—C₂H₅ to protect 1, 2, 3, 4 or more hydroxyl groups, or

a glycol or polyethyleneglycol or a derivative, e.g., propylene glycol, ethylene glycol, 1,4-butylene glycol, 1,3-butylene glycol, 1,2-butylene glycol, —O—C(O)—O—(CH₂CH₂O)ₙ—H, —C(O)—CH₂—O—C(O)—O—(CH₂CH₂O)ₙ—H or —O—(CH₂CH₂O)ₙ—H, where n is 1, 2, 3, 4, 5, 6, 7, 8, 9 or 10, or

an acetal or spiro ring, e.g., —O—CH₂—O—, —O—(CH₂)₂—O—, —O—(CH₂)₃—O— or —[C(R³⁶)₂]₁₋₄—O—, —O—C(O)—CH₂—, —O—C(O)—CH₂—CH₂—, —O—C(O)—CH₂—CH₂—CH₂—, —O—C(O)—CHR¹⁰—, —O—C(O)—CHR¹⁰—CHR¹⁰—, —O—C(O)—(CHR¹⁰)₃—, —NH—(CH₂)₂—O—, —NH—(CH₂)₂—NH—, —NH—(CH₂)₂—S—, —CH₂—N═CH—NH—, —NH—(CH₂)₃—O—, —NH—(CH₂)₃—S—, —NH—(CH₂)₃—O—, where R¹⁰ are independently selected and optionally independently are —H, —F, —Cl, —Br, —I, —CH₃, —C₂H₅, —CF₃, —C₂F₅, —CH₂CF₃, —OH, —CN, —SCN, —OCH₃ or —OC₂H₅, and where each R³⁶ independently is —H, —F, —Cl, —Br, —I or an organic moiety such as C1-C10 optionally substituted alkyl (e.g., methyl or ethyl), C2-10 alkenyl, aryl or a heterocycle, any of which are optionally substituted as described herein, e.g., —CF₃ or —CH₂OH, or

thioacetal, e.g., —S—CH₂—O—, —S—(CH₂)₂—O—, —S—(CH₂)₃—O—, —S—CH₂—S—, —S—(CH₂)₂—S—, —S—(CH₂)₃—S— or —S—[C(R³⁶)₂]₁₋₄—S— where each R³⁶ independently is —H, —F, —Cl, —Br, —I or an organic moiety such as C1-C10 optionally substituted alkyl (e.g., methyl or ethyl), C2-10 alkenyl, aryl or a heterocycle, any of which are optionally substituted as described herein, e.g., —CF₃ or —CH₂OH. The salts, ionized forms and solvates of any of these moieties are also included, e.g., where a group such as —NH₂ or —COOH is ionized to generate a moiety such as —NH₃⁺Cl⁻, —NH₃⁺Br⁻, —COO⁻Na⁺ or —COO⁻K⁺.

For any F1C structure, some embodiments are characterized by the presence of one or two independently selected substitutions at the 1-, 4-, 6-, and 12-positions or at R^{10A}, R^{10B}, R^{10C} and R^{10D} and optionally:

(a) R^{10E} (when present at the 5-position), R^{10F}, R^{10G} and R^{10H} are independently selected R¹⁰ groups in the α,β,α,α or β,β,α,α configurations respectively, R¹ is an oxygen-bonded, nitrogen-bonded or a sulfur-bonded moiety such as —OH, ═O, —SH, ═NOH, —NH(C1-C8 optionally substituted alkyl), an ester, an ether, a thioester, or a thioether, R^{1A} is —H, absent, a carbon-bonded moiety such as an acyl moiety, optionally substituted alkyl or optionally substituted alkylaryl, R² is a halogen or an oxygen-bonded or a sulfur-bonded moiety, R^{2A} is —H, absent, a carbon-bonded moiety, R³ is a halogen or an oxygen-bonded or a sulfur-bonded moiety, R^{3B} is —H, absent, a carbon-bonded moiety, R⁴ is a halogen, an oxygen-bonded or a sulfur-bonded moiety, R^{4A} is —H, absent, a carbon-bonded moiety such as an acyl moiety, optionally substituted alkyl or optionally substituted alkylaryl,

b) R^{10E} (if present), R^{10F}, R^{10G} and R^{10H} are independently selected R¹⁰ groups in the α,β,α,α or β,β,α,α configurations respectively, R^{1A} is —H, an oxygen-bonded, nitrogen-bonded or a sulfur-bonded moiety, R¹ is —H, a carbon-bonded moiety, R² is a halogen or an oxygen-bonded or a sulfur-bonded moiety, R^{2A} is —H, absent, a carbon-bonded moiety, R³ is a halogen or an oxygen-bonded or a sulfur-bonded moiety, R^{3B} is —H, absent, a carbon-bonded moiety, R⁴ is a halogen, an oxygen-bonded or a sulfur-bonded moiety, R^{4A} is —H, absent or a carbon-bonded moiety,

(c) R^{10E} (if present), R^{10F}, R^{10G} and R^{10H} are independently selected R¹⁰ groups in the α,β,α,α or β,β,α,α configurations respectively, R¹ is an oxygen-bonded, nitrogen-bonded or a sulfur-bonded moiety, R^{1A} is —H, absent or a carbon-bonded moiety, R² is a halogen or an oxygen-bonded or a sulfur-bonded moiety, R^{2A} is —H, absent or a carbon-bonded moiety, R³ is a halogen or an oxygen-bonded or a sulfur-bonded moiety, R^{3B} is —H, absent or a carbon-bonded moiety, R^{4A} is a halogen, an oxygen-bonded or a sulfur-bonded moiety, R⁴ is —H, a halogen or a carbon-bonded moiety,

(d) R^{10E} (if present), R^{10F}, R^{10G} and R^{10H} are independently selected R¹⁰ groups in the α,β,α,α or β,β,α,α configurations respectively, R¹ is an oxygen-bonded, nitrogen-bonded or a sulfur-bonded moiety, R^{1A} is —H, absent, a carbon-bonded moiety, R² is a halogen or an oxygen-bonded or a sulfur-bonded moiety, R^{2A} is —H, absent or a carbon-bonded moiety, R³ is a halogen or an oxygen-bonded or a sulfur-bonded moiety, R^{3B} is —H, absent or a carbon-bonded moiety, R⁴is a halogen, an oxygen-bonded or a sulfur-bonded moiety, R^{4A} is —H, absent or a carbon-bonded moiety,

(e) R^{10E} (if present), R^{10F}, R^{10G} and R^{10H} are independently selected R¹⁰ groups in the α,β,α,α or β,β,α,α configurations respectively, R¹ is an oxygen-bonded, nitrogen-bonded or a sulfur-bonded moiety, R^{1A} is —H, absent or a carbon-bonded moiety, R² is a halogen or an oxygen-bonded or a sulfur-bonded moiety, R^{2A} is —H, absent or a carbon-bonded moiety, R^{3B} is a halogen or an oxygen-bonded or a sulfur-bonded moiety, R³ is —H, a carbon-bonded moiety, R⁴ is a halogen, an oxygen-bonded or a sulfur-bonded moiety, R^{4A} is —H, absent or a carbon-bonded moiety,

(f) R^{10E} (if present), R^{10F}, R^{10G} and R^{10H} are independently selected R¹⁰ groups in the α,β,α,α or β,β,α,α configurations respectively, R^{1A} is —H, an oxygen-bonded, nitrogen-bonded or a sulfur-bonded moiety, R¹ is —H, a carbon-bonded moiety, R² is a halogen or an oxygen-bonded or a sulfur-bonded moiety, R^{2A} is —H, absent or a carbon-bonded moiety, R³ is a halogen or an oxygen-bonded or a sulfur-bonded moiety, R^{3B} is —H, absent or a carbon-bonded moiety, R^{4A} is a halogen, an oxygen-bonded or a sulfur-bonded moiety, R⁴ is —H, a carbon-bonded moiety, or

(g) R^{10E} (if present), R^{10F}, R^{10G} and R^{10H} are independently selected R¹⁰ groups in the α,β,α,α or β,β,α,α configurations respectively, R¹ is a halogen or an oxygen-bonded, nitrogen-bonded, carbon bonded or a sulfur-bonded moiety, R^{1A} is —H, a carbon-bonded or nitrogen-bonded moiety and R², R^{2A}, R³ R^{3B}, R⁴ and R⁴A are as described any of in the foregoing embodiments or elsewhere herein. In any of these embodiments, R⁵-R⁹ are independently selected moieties as described herein and the oxygen-bonded, nitrogen-bonded, carbon bonded or sulfur-bonded moieties at R¹, R^{1A}, R², R^{2A}, R³, R^{3B}, R⁴, and R^{4A} include atoms or groups described herein. These embodiments contain formula B, C, D, E, F and G compounds wherein one or two of R¹, R^{1A}, R², R^{2A}, R³, R^{3B}, R⁴, and R^{4A} are independently selected nitrogen-bonded moieties, one, two or three of R¹, R^{1A}, R², R^{2A}, R³, R^{3B}, R⁴, and R^{4A} are independently selected carbon-bonded moieties and one, two, three, four or five of R², R^{2A}, R³, R^{3B}, R⁴, and R^{4A} are independently selected or halogen atoms or oxygen-bonded or sulfur-bonded moieties.

Any of the F1C can contain two independently selected R⁴ groups, i.e., no 16-17 or 13-17 double bond is present, and both are the same, such as optionally substituted 20 alkyl, halogen, ether, ester, thioether, thioester, e.g., —OR^{PR}, —SR^{PR}, —F, —Cl, —Br, —I, alkyl, ethyl, methoxy, ethoxy acetate or propionate. In other embodiments, each R⁴ is an independently selected dissimilar moiety, e.g., independently selected —H, —OH, —OR^{PR}, an ester (e.g., —OC(O)—CH₃, —OC(O)—C₂H₅, —OC(O)—C3 alkyl, —OC(O)—C4 alkyl,), ether (e.g., —OCH₃, —OC₂H₅, —OCH₂CH₂CH₃, or —OCH(CH₃)CH₃, —O—C4 alkyl, —O—C5 alkyl or —O—C6 alkyl), a thioester, a thioether, an acyl moiety, a carbonate, a carbamate an amide, a monosaccharide, a disaccharide, or an amino acid, optionally substituted alkyl, optionally substituted alkenyl, optionally substituted alkynyl or another moiety described herein.

For any F1C, e.g., any F1C structure or any F1C species or genus described in the compound groups disclosed below, examples of dissimilar R⁴ and R^{4A} moieties at the 17-position include (a-ester, P-optionally substituted alkynyl), (β-ester, α-optionally substituted alkynyl), (α-thioester, β-optionally substituted alkynyl), (β-thioester, α-optionally substituted alkynyl), (α-ester, β-optionally substituted alkenyl), (β-ester, α-optionally substituted alkenyl), (α-thioester, β-optionally substituted alkenyl), (β-thioester, α-optionally substituted alkenyl), (α-optionally substituted alkyl, β-ester), (β-optionally substituted alkyl, α-ester), (α-optionally substituted alkyl, β-optionally substituted amine), (β-optionally substituted alkyl, α-optionally substituted amine), (α-optionally substituted alkyl, β-halogen)-, (β-optionally substituted alkyl, α-halogen), (α-halogen, β-ether), (β-halogen, α-ether), (α-halogen, β-optionally substituted alkyl), (β-halogen, α-optionally substituted alkyl), (β-ester, α-acyl), (α-ester, β-acyl), (β-ester, α-C(O)—C1-C10 optionally substituted alkyl), (α-ester, β-C(O)—C1-C10 optionally substituted alkyl), (β-thioester, α-C(O)—C1-C10 optionally substituted alkyl), (α-thioester, β-C(O)—C1-C10 optionally substituted alkyl), (β-OH, α-ester), (α-OH, β-ester), (β-OH, α-ether), (α-OH, β-ether), (β-OH, α-acyl), (α-OH, β-acyl), (α-halogen, β-OR^{PR}), (β-halogen, α-OR^{PR}), (α-F, β-ester), (β-F, α-ester), (α-F, β-ether), (β-F, α-ether), (α-Br, β-ether), (β-Br, α-ether), (α-OH, β-optionally substituted alkyl), (β-OH, α-optionally substituted alkyl), (α-OH, β-optionally substituted alkenyl), (β-OH, °-optionally substituted alkenyl), (α-OH, β-optionally substituted alkynyl), (β-OH, α-optionally substituted alkynyl), (α-OH, β-C≡CCH₂-halogen), (β-OH, α-C≡CCH₂-halogen), (α-OH, β-C≡C-halogen), (β-OH, α-C≡C-halogen), (β-epoxy, α-halogen, where the epoxy is formed with an adjacent steroid nucleus atom), (α-epoxy, pβhalogen), (α-cyclopropyl, β-halogen), (β-cyclopropyl, α-halogen), (α-cyclopropyl, β-optionally substituted alkyl), (β-cyclopropyl, α-optionally substituted alkyl), (α-optionally substituted alkyl, β-NH—C1-C8 optionally substituted alkyl), (β-optionally substituted alkyl, α-NH—C1-C8 optionally substituted alkyl), (α-ether, β-NH—C1-C8 optionally substituted alkyl), (β-ether, α-NH—C1-C8 optionally substituted alkyl), (α-thioester, β-NH—C1-C8 optionally substituted alkyl), (β-thioester, α-NH—C1-C8 optionally substituted alkyl), (α-ester, β-NH—C1-C8 optionally substituted alkyl), (β-ester, α-NH—C1-C8 optionally substituted alkyl), (α-C(O)CH₃, β-NH—C1-C8 optionally substituted alkyl), (β-C(O)CH₃, α-NH—C1-C8 optionally substituted alkyl), (α-OH, β-NH—C1-C8 optionally substituted alkyl), (β-OH, α-NH—C1-C8 optionally substituted alkyl) and other combinations of groups that are within the scope of R⁴ and R^{4A}. Such moieties, which are the same or different can also be at 1, 2, 3 or more R¹, R², R³ or R¹⁰ variable groups, including the R¹⁰ variable groups at R⁷, R⁸, R⁹ or R¹¹ or the 4-position.

Specific dissimilar R⁴ moieties include, e.g., (α-OH, β-CH₃), (β-OH, α-CH₃), (α-OH, β-C₂H₅), (β-OH, α-C₂H₅), (α-CH₃, β-OCH₃), (β-CH₃, α-OCH₃), (α-C₂H₅, β-C₂H₅, α-OCH₃), (α-OH, β-CHCH₂), (β-OH, α-CHCH₂), (α-OH, β-CCCH₃), (β-OH, α-CCCH₃), (α-OH, β-CCCH₂OH), (β-OH, α-CCCH₂OH), (α-OH, β-CCH), (β-OH, α-CCH), (α-OH, β-C(O)CH₃), (β-OH, α-C(O)CH₃), (α-OH, β-C(O)CH₂OH), (β-OH, α-C(O)CH₂OH), (α-OH, β-C(O)CH₂Cl), (β-OH, α-C(O)CH₂Cl), (α-CH₃, β-OC(O)CH₃), (β-CH₃, α-OC(O)CH₃), (α-C₂H₅, β-OC(O)CH₃), (β-C₂H₅, α-OC(O)CH₃), (α-C₃H₇, β-OC(O)CH₃), (β-C₃H₇, α-OC(O)CH₃), (α-C₄H₉, β-OC(O)CH₃), (β-C₄H₉, α-OC(O)CH₃), (α-C₂H₃, β-OC(O)CH₃), (β-C₂H₃, α-OC(O)CH₃), (α-C₂H₄OH, β-OC(O)CH₃), (β-C₂H₄OH, α-OC(O)CH₃), (α-C₃H₅, β-OC(O)CH₃), (β-C₃H₅, α-OC(O)CH₃), (α-C₄H₇, β-OC(O)CH₃), (β-C₄H₇, α-OC(O)CH₃), (α-C₃H₃, β-OC(O)CH₃), (β-C₃H₃, α-OC(O)CH₃), (α-C₄H₅, β-OC(O)CH₃), (β-C₄H₅, α-OC(O)CH₃), (α-CH₃, β-OC(O)C₂H₅), (β-CH₃, α-OC(O)C₂H₅), (αC₂H₅, β-OC(O)C₂H₅), (β-C₂H₅, α-OC(O)C₂H₅), (α-C₃H₇, β-OC(O)C₂H₅), (β-C₃H₇, α-OC(O)C₂H₅), (α-C₄H₉, β-OC(O)C₂H₅), (β-C₄H₉, α-OC(O)C₂H₅), (α-C₂H₃, β-OC(O)C₂H₅), (β-C₂H₃, α-OC(O)C₂H₅), (α-C₂H₄OH, β-OC(O)C₂H₅), (β-C₂H₄OH, α-OC(O)C₂H₅), (α-C₃H₅, β-OC(O)C₂H₅), (β-C₃H₅, α-OC(O)C₂H₅), (α-C₄H₇, β-OC(O)C₂H₅), (β-C₄H₇, α-OC(O)C₂H₅), (α-C₃H₃, β-OC(O)C₂H₅), (β-C₃H₃, α-OC(O)C₂H₅), (α-C₄H₅, β-OC(O)C₂H₅), (β-C₄H₅, α-OC(O)C₂H₅), (α-C(O)CH₃, β-OC(O)CH₃), (β-C(O)CH₃, 60 -OC(O)CH₃), (α-C(O)C₂H₅, β-OC(O)CH₃), (β-C(O)C₂H₅, α-OC(O)CH₃), (α-CH₃, β-SC(O)CH₃), (β-CH₃, α-SC(O)CH₃), (α-C₂H₅, β-SC(O)CH₃), (β-C₂H₅, α-SC(O)CH₃), (α-C₃H₇, β-SC(O)CH₃), (β-C₃H₇, α-SC(O)CH₃), (α-C₄H₉, β-SC(O)CH₃), (β-C₄H₉, α-SC(O)CH₃), (α-C₂H₃, β-SC(O)CH₃), (β-C₂H₃, α-SC(O)CH₃), (α-C₂H₄OH, β-SC(O)CH₃), (β-C₂H₄OH, α-SC(O)CH₃), (α-C₃H₅, β-SC(O)CH₃), (β-C₃H₅, α-SC(O)CH₃), (α-C₄H₇, β-SC(O)CH₃), (β-C₄H₇, α-SC(O)CH₃), (α-C₃H₃, β-SC(O)CH₃), (β-C₃H₃, α-SC(O)CH₃), (α-C₄H₅, β-SC(O)CH₃), (β-C₄H₅, α-SC(O)CH₃), (α-CH₃, β-SC(O)C₂H₅), (β-CH₃, α-SC(O)C₂H₅), (α-C₂H₅, β-SC(O)C₂H₅), (β-C₂H₅, α-SC(O)C₂H₅), (α-C₃H₇, α-SC(O)C₂H₅), (α-C₄H₉, β-SC(O)C₂H₅), (β-C₄H₉, α-SC(O)C₂H₃, β-SC(O)C₂H₅), (β-C₂H₃, α-SC(O)C₂H₅), (α-C₂H₄OH, β-SC(O)C₂H₅), (β-C₂H₄OH, α-SC(O)C₂H₅), (α-C₃H₅, β-SC(O)C₂H₅), (β-C₃H₅, α-SC(O)C₂H₅), (α-C₄H₇, β-SC(O)C₂H₅), (β-C₄H₇, α-SC(O)C₂H₅), (α-C₃H₃, β-SC(O)C₂H₅), (β-C₃H₃, α-SC(O)C₂H₅), (α-C₄H₅, β-SC(O)C₂H₅), (β-C₄H₅, α-SC(O)C₂H₅), (α-C(O)CH₃, β-SC(O)CH₃), (β-C(O)CH₃, α-SC(O)CH₃), (α-C(O)C₂H₅, β-SC(O)CH₃), (β-C(O)C₂H₅, α-SC(O)CH₃), (α-C(O)CH₃, β-NH-CH₃), (β-C(O)CH₃, α-NH—CH₃), (α-OH, β-NH—CH₃), (β-OH, α-NH—CH₃), (α-C(O)CH₃, β-N(CH₃)₂), (β-C(O)CH₃, α-N(CH₃)₂), (α-OH, β-N(CH₃)₂), (β-OH, α-N(CH₃)₂), (α-C(O)CH₃, β-N(C₂H₅)₂), α-N(C₂H₅)₂), (α-OH, β-N(C₂H₅)₂), (β-OH, α-N(C₂H₅)₂), (β-epoxy, α-H), (α-epoxy, β-H), (β-epoxy, α-Br), (α-epoxy, β-Br), (β-epoxy, α-F), (α-epoxy, β-F), (β-cyclopropyl, α-H), (α-cyclopropyl, β-H), (β-cyclopropyl, α-F) and (α-cyclopropyl, β-F). For moieties that contain an epoxy, cyclopropyl or other cyclic moiety, the cyclic moiety can be formed with an adjacent variable group, e.g., R³ or R³⁸.

As is apparent from the foregoing disclosure, these or other dissimilar moieties can also be present at one or more of, e.g., the 2-, 3-, 7-, 11-, 15- or 16-positions. Thus, other variable groups such as R¹, R³ or R¹⁰ can be any of the same or dissimilar pairs of moieties as described above for the R⁴ moieties.

In the F1Cs, one or both of R⁵ or R⁶ can independently be —H, —CH₂SH, —CHO, —CH₂NRPR, —CH₂NH₂, —C₄H₉, —C₃H₇, —C₂H₅, —CH₃, —C₂H₄OH, —C₂H₄SH, —C₂H₄NH₂, —CH₂CHO, —CH₂CH₂NR^{PR}, —CH₂CH₂OH, —CH₂CH₂SH, —CH₂CH₂C₆H₅, —CH₂C₆H₅, —C₆H₅ or optionally substituted alkyl wherein any phenyl (C₆H₅) moiety in the foregoing groups is optionally substituted at the phenyl ring with 1, 2, 3, 4 or 5 moieties independently selected from those described for esters herein and including C1-C6 alkyl (optionally substituted with 1 or 2 independently selected —OH, —SH, —O—, —S— or —NH—) Cl -C6 alkoxy, —F, —Cl, —Br, —I, —CN, —NO₂, —OH, —SH, —COOR^{PR}, —NHR^{PR} and —C(O)—C1-C6 alkyl Typically R⁵ or R⁶ are both in the β-configuration, but they may be in, e.g., the α,β,β,α or α,αconfigurations respectively.

F1C embodiments also include compounds where 1,2 or more of, e.g., R¹, R², R³, R⁴ and R¹⁰ are a lipid moiety such as a fatty acid, a monoacylglyceride, a diacylglyceride, a phospholipid, a glycolipid, a sphingolipid or a glycerophospholipid that is esterified, linked through an ether (—O—) or acyl moiety or otherwise bonded to the F1C. Exemplary fatty acid esters include —C(O)—(CH₂)ₘ—H where m is 4, 5, 6, 7, 8, 9, 10, 11, 12, 13, 15, 17, 19 or 21 and —C(O)—(CH₂)ₙ—CH═CH—(CH₂)ₙ—H where each n independently is 1, 2, 3, 4, 5, 6, 7 or 8. Other lipid moieties that can be bonded to the steroid include phosphatidic acid, phosphatidylethanolamine, phosphatidylcholine, phosphatidylserine and phosphatidylglycerol. The lipid moiety may be bonded to the steroid through a hydroxyl or oxygen, phosphate, sulfate or amine at a variable group. Such lipid moieties may be bonded to any of the F1Cs or genera of F1Cs disclosed herein.

Specific F1Cs that can be used in the clinical treatments and other methods described herein include the following groups of compounds.

Group 1. Exemplary embodiments include the formula 1 compounds named according to the compound structure designations given in Tables A and B below. Each compound named in Table B is depicted as a compound having the structure

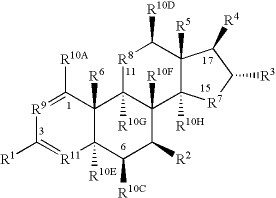

where R⁵ and R⁶ are both —CH₃, there is a double bond at the 1-2- and 3-4 positions, R⁷, R⁸ and R⁹ are all —CH₂— or ═CH—, R¹¹ is ═CR^{10B}, R^{10A}, R^{10B}, R^{10C}, R^{10D}, R^{10E}, R^{10F}, R^{10G} and R^{10H} are all —H and R¹, R², R³ and R⁴ are the substituents designated in Table A. The compounds named according to Tables A and B are referred to as “group 1” compounds.

Compounds named in Table B are named by numbers assigned to R¹, R² R³ and R⁴ according to the following compound naming convention, R¹.R².R³.R⁴, using the numbered chemical substituents in Table A. Each Table A number specifies a different structure for each of R¹, R², R³ and R⁴. When R¹, R², R³ or R⁴ is a divalent moiety, e.g., ═O, the hydrogen at the corresponding position is absent. Thus, the group 1 compound named 1.2.4.9 is a group 1 compound with a β-hydroxyl bonded to carbons at the 3- and 7-positions (the variable groups R¹ and R² respectively), an α-fluorine bonded to carbon 16 (the variable group R³) and acetate at carbon 17 (the variable group R⁴), i.e., 1.2.4.9 is 3,7β,17β-trihydroxy-16α-fluoroandrost-1,3-diene, which has the structure

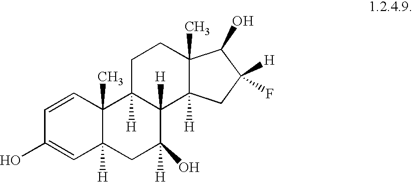

Similarly, group 1 compound 1.2.4.1 is 3,7β-dihydroxy-16α-fluoro-17β-aminoandrost-1,3-diene, group 1 compound 1.1.5.9 is 3,17β-dihydroxyandrost-1,3-diene, 1.1.7.1, which is 3-hydroxy-16α-acetoxy-17β-aminoandrost-1,3-diene and compound 1.1.4.10, which is 3-hydroxy-16α-fluoro-17β-acetoxyandrost-1,3-diene. Other exemplary group 1 compounds include 3,17β-dihydroxy-7β-acetoxyandrost-1,3-diene, 3,17β-dihydroxy-7β-methylandrost-1,3-diene, 3,17β-dihydroxy-7β-methoxyandrost-1,3-diene, 3,7β,17β-trihydroxyandrost-1,3-diene, 3-amino-17β-hydroxyandrost-1,3-diene, 3-amino-7β,17β-dihydroxyandrost-1,3-diene, 3-hydroxy-17β-aminoandrost-1,3-diene, 3,7β-dihydroxy-17β-aminoandrost-1,3-diene, 3,17β-dihydroxy-7β-aminoandrost-1,3-diene, 3-hydroxy-7β,17β-diacetylaminoandrost-1,3-diene, 3-hydroxy-7β,17β-dimethylaminoandrost-1,3-diene and 16α-hydroxy, 16α-methyl, 16α-amino, 16α-aminomethyl, 16α-acetate and 16α-halo analogs of any of these compounds.

TABLE A

| | |
|---|---|
| R¹ | R² |

| | | | |
|---|---|---|---|
| 1 | —OH | 1 | —H |
| 2 | —OCH₃ | 2 | —OH |
| 3 | —SH | 3 | —OCH₃ |
| 4 | —O—C(O)—CH₃ | 4 | —N(CH₃)₂ |
| 5 | —NHCH₃ | 5 | —CH₃ |
| 6 | —NH₂ | 6 | —NH₂ |
| 7 | —NH—C(O)—CH₃ | 7 | —NH—C(O)—CH₃ |
| 8 | —N(CH₃)₂ | 8 | —NH—CH₃ |
| 9 | —O—D-β-glucoside | 9 | —O—C(O)—CH₃ |
| 10 | —O—S(O)(OH)—OH | 10 | —SH |

| | |
|---|---|
| R³ | R⁴ |

| | | | |
|---|---|---|---|
| 1 | —Br | 1 | —NH₂ |
| 2 | —Cl | 2 | —NH—C(O)—CH₃ |
| 3 | —I | 3 | —NH—C(O)—OCH₃ |
| 4 | —F | 4 | —NH—CH₃ |
| 5 | —H | 5 | —N(CH₃)₂ |
| 6 | —OH | 6 | —OCH₃ |
| 7 | —O—C(O)—CH₃ | 7 | —O—S(O)(OH)—OH |
| 8 | —CH₃ | 8 | —O—C(O)—CH₂CH₃ |
| 9 | —NH₂ | 9 | —OH |
| 10 | —NHCH₃ | 10 | —O—C(O)—CH₃ |

TABLE B
1.1.1.1, 1.1.1.2, 1.1.1.3, 1.1.1.4, 1.1.1.5, 1.1.1.6, 1.1.1.7, 1.1.1.8, 1.1.1.9, 1.1.1.10, 1.1.2.1,
1.1.2.2, 1.1.2.3, 1.1.2.4, 1.1.2.5, 1.1.2.6, 1.1.2.7, 1.1.2.8, 1.1.2.9, 1.1.2.10, 1.1.3.1, 1.1.3.2,
1.1.3.3, 1.1.3.4, 1.1.3.5, 1.1.3.6, 1.1.3.7, 1.1.3.8, 1.1.3.9, 1.1.3.10, 1.1.4.1, 1.1.4.2, 1.1.4.3,
1.1.4.4, 1.1.4.5, 1.1.4.6, 1.1.4.7, 1.1.4.8, 1.1.4.9, 1.1.4.10, 1.1.5.1, 1.1.5.2, 1.1.5.3, 1.1.5.4,
1.1.5.5, 1.1.5.6, 1.1.5.7, 1.1.5.8, 1.1.5.9, 1.1.5.10, 1.1.6.1, 1.1.6.2, 1.1.6.3, 1.1.6.4, 1.1.6.5,
1.1.6.6, 1.1.6.7, 1.1.6.8, 1.1.6.9, 1.1.6.10, 1.1.7.1, 1.1.7.2, 1.1.7.3, 1.1.7.4, 1.1.7.5, 1.1.7.6,
1.1.7.7, 1.1.7.8, 1.1.7.9, 1.1.7.10, 1.1.8.1, 1.1.8.2, 1.1.8.3, 1.1.8.4, 1.1.8.5, 1.1.8.6, 1.1.8.7,
1.1.8.8, 1.1.8.9, 1.1.8.10, 1.1.9.1, 1.1.9.2, 1.1.9.3, 1.1.9.4, 1.1.9.5, 1.1.9.6, 1.1.9.7, 1.1.9.8,
1.1.9.9, 1.1.9.10, 1.1.10.1, 1.1.10.2, 1.1.10.3, 1.1.10.4, 1.1.10.5, 1.1.10.6, 1.1.10.7, 1.1.10.8,
1.1.10.9, 1.1.10.10, 1.2.1.1, 1.2.1.2, 1.2.1.3, 1.2.1.4, 1.2.1.5, 1.2.1.6, 1.2.1.7, 1.2.1.8, 1.2.1.9,
1.2.1.10, 1.2.2.1, 1.2.2.2, 1.2.2.3, 1.2.2.4, 1.2.2.5, 1.2.2.6, 1.2.2.7, 1.2.2.8, 1.2.2.9, 1.2.2.10,
1.2.3.1, 1.2.3.2, 1.2.3.3, 1.2.3.4, 1.2.3.5, 1.2.3.6, 1.2.3.7, 1.2.3.8, 1.2.3.9, 1.2.3.10, 1.2.4.1,
1.2.4.2, 1.2.4.3, 1.2.4.4, 1.2.4.5, 1.2.4.6, 1.2.4.7, 1.2.4.8, 1.2.4.9, 1.2.4.10, 1.2.5.1, 1.2.5.2,
1.2.5.3, 1.2.5.4, 1.2.5.5, 1.2.5.6, 1.2.5.7, 1.2.5.8, 1.2.5.9, 1.2.5.10, 1.2.6.1, 1.2.6.2, 1.2.6.3,
1.2.6.4, 1.2.6.5, 1.2.6.6, 1.2.6.7, 1.2.6.8, 1.2.6.9, 1.2.6.10, 1.2.7.1, 1.2.7.2, 1.2.7.3, 1.2.7.4,
1.2.7.5, 1.2.7.6, 1.2.7.7, 1.2.7.8, 1.2.7.9, 1.2.7.10, 1.2.8.1, 1.2.8.2, 1.2.8.3, 1.2.8.4, 1.2.8.5,
1.2.8.6, 1.2.8.7, 1.2.8.8, 1.2.8.9, 1.2.8.10, 1.2.9.1, 1.2.9.2, 1.2.9.3, 1.2.9.4, 1.2.9.5, 1.2.9.6,
1.2.9.7, 1.2.9.8, 1.2.9.9, 1.2.9.10, 1.2.10.1, 1.2.10.2, 1.2.10.3, 1.2.10.4, 1.2.10.5, 1.2.10.6,
1.2.10.7, 1.2.10.8, 1.2.10.9, 1.2.10.10, 1.3.1.1, 1.3.1.2, 1.3.1.3, 1.3.1.4, 1.3.1.5, 1.3.1.6, 1.3.1.7,
1.3.1.8, 1.3.1.9, 1.3.1.10, 1.3.2.1, 1.3.2.2, 1.3.2.3, 1.3.2.4, 1.3.2.5, 1.3.2.6, 1.3.2.7, 1.3.2.8,
1.3.2.9, 1.3.2.10, 1.3.3.1, 1.3.3.2, 1.3.3.3, 1.3.3.4, 1.3.3.5, 1.3.3.6, 1.3.3.7, 1.3.3.8, 1.3.3.9,
1.3.3.10, 1.3.4.1, 1.3.4.2, 1.3.4.3, 1.3.4.4, 1.3.4.5, 1.3.4.6, 1.3.4.7, 1.3.4.8, 1.3.4.9, 1.3.4.10,
1.3.5.1, 1.3.5.2, 1.3.5.3, 1.3.5.4, 1.3.5.5, 1.3.5.6, 1.3.5.7, 1.3.5.8, 1.3.5.9, 1.3.5.10, 1.3.6.1,
1.3.6.2, 1.3.6.3, 1.3.6.4, 1.3.6.5, 1.3.6.6, 1.3.6.7, 1.3.6.8, 1.3.6.9, 1.3.6.10, 1.3.7.1, 1.3.7.2,
1.3.7.3, 1.3.7.4, 1.3.7.5, 1.3.7.6, 1.3.7.7, 1.3.7.8, 1.3.7.9, 1.3.7.10, 1.3.8.1, 1.3.8.2, 1.3.8.3,
1.3.8.4, 1.3.8.5, 1.3.8.6, 1.3.8.7, 1.3.8.8, 1.3.8.9, 1.3.8.10, 1.3.9.1, 1.3.9.2, 1.3.9.3, 1.3.9.4,
1.3.9.5, 1.3.9.6, 1.3.9.7, 1.3.9.8, 1.3.9.9, 1.3.9.10, 1.3.10.1, 1.3.10.2, 1.3.10.3, 1.3.10.4, 1.3.10.5,
1.3.10.6, 1.3.10.7, 1.3.10.8, 1.3.10.9, 1.3.10.10, 1.4.1.1, 1.4.1.2, 1.4.1.3, 1.4.1.4, 1.4.1.5, 1.4.1.6,
1.4.1.7, 1.4.1.8, 1.4.1.9, 1.4.1.10, 1.4.2.1, 1.4.2.2, 1.4.2.3, 1.4.2.4, 1.4.2.5, 1.4.2.6, 1.4.2.7,
1.4.2.8, 1.4.2.9, 1.4.2.10, 1.4.3.1, 1.4.3.2, 1.4.3.3, 1.4.3.4, 1.4.3.5, 1.4.3.6, 1.4.3.7, 1.4.3.8,
1.4.3.9, 1.4.3.10, 1.4.4.1, 1.4.4.2, 1.4.4.3, 1.4.4.4, 1.4.4.5, 1.4.4.6, 1.4.4.7, 1.4.4.8, 1.4.4.9,
1.4.4.10, 1.4.5.1, 1.4.5.2, 1.4.5.3, 1.4.5.4, 1.4.5.5, 1.4.5.6, 1.4.5.7, 1.4.5.8, 1.4.5.9, 1.4.5.10,
1.4.6.1, 1.4.6.2, 1.4.6.3, 1.4.6.4, 1.4.6.5, 1.4.6.6, 1.4.6.7, 1.4.6.8, 1.4.6.9, 1.4.6.10, 1.4.7.1,
1.4.7.2, 1.4.7.3, 1.4.7.4, 1.4.7.5, 1.4.7.6, 1.4.7.7, 1.4.7.8, 1.4.7.9, 1.4.7.10, 1.4.8.1, 1.4.8.2,
1.4.8.3, 1.4.8.4, 1.4.8.5, 1.4.8.6, 1.4.8.7, 1.4.8.8, 1.4.8.9, 1.4.8.10, 1.4.9.1, 1.4.9.2, 1.4.9.3,
1.4.9.4, 1.4.9.5, 1.4.9.6, 1.4.9.7, 1.4.9.8, 1.4.9.9, 1.4.9.10, 1.4.10.1, 1.4.10.2, 1.4.10.3, 1.4.10.4,
1.4.10.5, 1.4.10.6, 1.4.10.7, 1.4.10.8, 1.4.10.9, 1.4.10.10, 1.5.1.1, 1.5.1.2, 1.5.1.3, 1.5.1.4,
1.5.1.5, 1.5.1.6, 1.5.1.7, 1.5.1.8, 1.5.1.9, 1.5.1.10, 1.5.2.1, 1.5.2.2, 1.5.2.3, 1.5.2.4, 1.5.2.5,
1.5.2.6, 1.5.2.7, 1.5.2.8, 1.5.2.9, 1.5.2.10, 1.5.3.1, 1.5.3.2, 1.5.3.3, 1.5.3.4, 1.5.3.5, 1.5.3.6,
1.5.3.7, 1.5.3.8, 1.5.3.9, 1.5.3.10, 1.5.4.1, 1.5.4.2, 1.5.4.3, 1.5.4.4, 1.5.4.5, 1.5.4.6, 1.5.4.7,
1.5.4.8, 1.5.4.9, 1.5.4.10, 1.5.5.1, 1.5.5.2, 1.5.5.3, 1.5.5.4, 1.5.5.5, 1.5.5.6, 1.5.5.7, 1.5.5.8,
1.5.5.9, 1.5.5.10, 1.5.6.1, 1.5.6.2, 1.5.6.3, 1.5.6.4, 1.5.6.5, 1.5.6.6, 1.5.6.7, 1.5.6.8, 1.5.6.9,
1.5.6.10, 1.5.7.1, 1.5.7.2, 1.5.7.3, 1.5.7.4, 1.5.7.5, 1.5.7.6, 1.5.7.7, 1.5.7.8, 1.5.7.9, 1.5.7.10,
1.5.8.1, 1.5.8.2, 1.5.8.3, 1.5.8.4, 1.5.8.5, 1.5.8.6, 1.5.8.7, 1.5.8.8, 1.5.8.9, 1.5.8.10, 1.5.9.1,
1.5.9.2, 1.5.9.3, 1.5.9.4, 1.5.9.5, 1.5.9.6, 1.5.9.7, 1.5.9.8, 1.5.9.9, 1.5.9.10, 1.5.10.1, 1.5.10.2,
1.5.10.3, 1.5.10.4, 1.5.10.5, 1.5.10.6, 1.5.10.7, 1.5.10.8, 1.5.10.9, 1.5.10.10, 1.6.1.1, 1.6.1.2,
1.6.1.3, 1.6.1.4, 1.6.1.5, 1.6.1.6, 1.6.1.7, 1.6.1.8, 1.6.1.9, 1.6.1.10, 1.6.2.1, 1.6.2.2, 1.6.2.3,
1.6.2.4, 1.6.2.5, 1.6.2.6, 1.6.2.7, 1.6.2.8, 1.6.2.9, 1.6.2.10, 1.6.3.1, 1.6.3.2, 1.6.3.3, 1.6.3.4,
1.6.3.5, 1.6.3.6, 1.6.3.7, 1.6.3.8, 1.6.3.9, 1.6.3.10, 1.6.4.1, 1.6.4.2, 1.6.4.3, 1.6.4.4, 1.6.4.5,
1.6.4.6, 1.6.4.7, 1.6.4.8, 1.6.4.9, 1.6.4.10, 1.6.5.1, 1.6.5.2, 1.6.5.3, 1.6.5.4, 1.6.5.5, 1.6.5.6,
1.6.5.7, 1.6.5.8, 1.6.5.9, 1.6.5.10, 1.6.6.1, 1.6.6.2, 1.6.6.3, 1.6.6.4, 1.6.6.5, 1.6.6.6, 1.6.6.7,
1.6.6.8, 1.6.6.9, 1.6.6.10, 1.6.7.1, 1.6.7.2, 1.6.7.3, 1.6.7.4, 1.6.7.5, 1.6.7.6, 1.6.7.7, 1.6.7.8,
1.6.7.9, 1.6.7.10, 1.6.8.1, 1.6.8.2, 1.6.8.3, 1.6.8.4, 1.6.8.5, 1.6.8.6, 1.6.8.7, 1.6.8.8, 1.6.8.9,
1.6.8.10, 1.6.9.1, 1.6.9.2, 1.6.9.3, 1.6.9.4, 1.6.9.5, 1.6.9.6, 1.6.9.7, 1.6.9.8, 1.6.9.9, 1.6.9.10,
1.6.10.1, 1.6.10.2, 1.6.10.3, 1.6.10.4, 1.6.10.5, 1.6.10.6, 1.6.10.7, 1.6.10.8, 1.6.10.9, 1.6.10.10,
1.7.1.1, 1.7.1.2, 1.7.1.3, 1.7.1.4, 1.7.1.5, 1.7.1.6, 1.7.1.7, 1.7.1.8, 1.7.1.9, 1.7.1.10, 1.7.2.1,
1.7.2.2, 1.7.2.3, 1.7.2.4, 1.7.2.5, 1.7.2.6, 1.7.2.7, 1.7.2.8, 1.7.2.9, 1.7.2.10, 1.7.3.1, 1.7.3.2,
1.7.3.3, 1.7.3.4, 1.7.3.5, 1.7.3.6, 1.7.3.7, 1.7.3.8, 1.7.3.9, 1.7.3.10, 1.7.4.1, 1.7.4.2, 1.7.4.3,
1.7.4.4, 1.7.4.5, 1.7.4.6, 1.7.4.7, 1.7.4.8, 1.7.4.9, 1.7.4.10, 1.7.5.1, 1.7.5.2, 1.7.5.3, 1.7.5.4,
1.7.5.5, 1.7.5.6, 1.7.5.7, 1.7.5.8, 1.7.5.9, 1.7.5.10, 1.7.6.1, 1.7.6.2, 1.7.6.3, 1.7.6.4, 1.7.6.5,
1.7.6.6, 1.7.6.7, 1.7.6.8, 1.7.6.9, 1.7.6.10, 1.7.7.1, 1.7.7.2, 1.7.7.3, 1.7.7.4, 1.7.7.5, 1.7.7.6,
1.7.7.7, 1.7.7.8, 1.7.7.9, 1.7.7.10, 1.7.8.1, 1.7.8.2, 1.7.8.3, 1.7.8.4, 1.7.8.5, 1.7.8.6, 1.7.8.7,
1.7.8.8, 1.7.8.9, 1.7.8.10, 1.7.9.1, 1.7.9.2, 1.7.9.3, 1.7.9.4, 1.7.9.5, 1.7.9.6, 1.7.9.7, 1.7.9.8,
1.7.9.9, 1.7.9.10, 1.7.10.1, 1.7.10.2, 1.7.10.3, 1.7.10.4, 1.7.10.5, 1.7.10.6, 1.7.10.7, 1.7.10.8,
1.7.10.9, 1.7.10.10, 1.8.1.1, 1.8.1.2, 1.8.1.3, 1.8.1.4, 1.8.1.5, 1.8.1.6, 1.8.1.7, 1.8.1.8, 1.8.1.9,
1.8.1.10, 1.8.2.1, 1.8.2.2, 1.8.2.3, 1.8.2.4, 1.8.2.5, 1.8.2.6, 1.8.2.7, 1.8.2.8, 1.8.2.9, 1.8.2.10,
1.8.3.1, 1.8.3.2, 1.8.3.3, 1.8.3.4, 1.8.3.5, 1.8.3.6, 1.8.3.7, 1.8.3.8, 1.8.3.9, 1.8.3.10, 1.8.4.1,
1.8.4.2, 1.8.4.3, 1.8.4.4, 1.8.4.5, 1.8.4.6, 1.8.4.7, 1.8.4.8, 1.8.4.9, 1.8.4.10, 1.8.5.1, 1.8.5.2,
1.8.5.3, 1.8.5.4, 1.8.5.5, 1.8.5.6, 1.8.5.7, 1.8.5.8, 1.8.5.9, 1.8.5.10, 1.8.6.1, 1.8.6.2, 1.8.6.3,
1.8.6.4, 1.8.6.5, 1.8.6.6, 1.8.6.7, 1.8.6.8, 1.8.6.9, 1.8.6.10, 1.8.7.1, 1.8.7.2, 1.8.7.3, 1.8.7.4,
1.8.7.5, 1.8.7.6, 1.8.7.7, 1.8.7.8, 1.8.7.9, 1.8.7.10, 1.8.8.1, 1.8.8.2, 1.8.8.3, 1.8.8.4, 1.8.8.5,
1.8.8.6, 1.8.8.7, 1.8.8.8, 1.8.8.9, 1.8.8.10, 1.8.9.1, 1.8.9.2, 1.8.9.3, 1.8.9.4, 1.8.9.5, 1.8.9.6,
1.8.9.7, 1.8.9.8, 1.8.9.9, 1.8.9.10, 1.8.10.1, 1.8.10.2, 1.8.10.3, 1.8.10.4, 1.8.10.5, 1.8.10.6,
1.8.10.7, 1.8.10.8, 1.8.10.9, 1.8.10.10, 1.9.1.1, 1.9.1.2, 1.9.1.3, 1.9.1.4, 1.9.1.5, 1.9.1.6, 1.9.1.7,
1.9.1.8, 1.9.1.9, 1.9.1.10, 1.9.2.1, 1.9.2.2, 1.9.2.3, 1.9.2.4, 1.9.2.5, 1.9.2.6, 1.9.2.7, 1.9.2.8,
1.9.2.9, 1.9.2.10, 1.9.3.1, 1.9.3.2, 1.9.3.3, 1.9.3.4, 1.9.3.5, 1.9.3.6, 1.9.3.7, 1.9.3.8, 1.9.3.9,
1.9.3.10, 1.9.4.1, 1.9.4.2, 1.9.4.3, 1.9.4.4, 1.9.4.5, 1.9.4.6, 1.9.4.7, 1.9.4.8, 1.9.4.9, 1.9.4.10,
1.9.5.1, 1.9.5.2, 1.9.5.3, 1.9.5.4, 1.9.5.5, 1.9.5.6, 1.9.5.7, 1.9.5.8, 1.9.5.9, 1.9.5.10, 1.9.6.1,
1.9.6.2, 1.9.6.3, 1.9.6.4, 1.9.6.5, 1.9.6.6, 1.9.6.7, 1.9.6.8, 1.9.6.9, 1.9.6.10, 1.9.7.1, 1.9.7.2,
1.9.7.3, 1.9.7.4, 1.9.7.5, 1.9.7.6, 1.9.7.7, 1.9.7.8, 1.9.7.9, 1.9.7.10, 1.9.8.1, 1.9.8.2, 1.9.8.3,
1.9.8.4, 1.9.8.5, 1.9.8.6, 1.9.8.7, 1.9.8.8, 1.9.8.9, 1.9.8.10, 1.9.9.1, 1.9.9.2, 1.9.9.3, 1.9.9.4,
1.9.9.5, 1.9.9.6, 1.9.9.7, 1.9.9.8, 1.9.9.9, 1.9.9.10, 1.9.10.1, 1.9.10.2, 1.9.10.3, 1.9.10.4, 1.9.10.5,
1.9.10.6, 1.9.10.7, 1.9.10.8, 1.9.10.9, 1.9.10.10, 1.10.1.1, 1.10.1.2, 1.10.1.3, 1.10.1.4, 1.10.1.5,
1.10.1.6, 1.10.1.7, 1.10.1.8, 1.10.1.9, 1.10.1.10, 1.10.2.1, 1.10.2.2, 1.10.2.3, 1.10.2.4, 1.10.2.5,
1.10.2.6, 1.10.2.7, 1.10.2.8, 1.10.2.9, 1.10.2.10, 1.10.3.1, 1.10.3.2, 1.10.3.3, 1.10.3.4, 1.10.3.5,
1.10.3.6, 1.10.3.7, 1.10.3.8, 1.10.3.9, 1.10.3.10, 1.10.4.1, 1.10.4.2, 1.10.4.3, 1.10.4.4, 1.10.4.5,
1.10.4.6, 1.10.4.7, 1.10.4.8, 1.10.4.9, 1.10.4.10, 1.10.5.1, 1.10.5.2, 1.10.5.3, 1.10.5.4, 1.10.5.5,
1.10.5.6, 1.10.5.7, 1.10.5.8, 1.10.5.9, 1.10.5.10, 1.10.6.1, 1.10.6.2, 1.10.6.3, 1.10.6.4, 1.10.6.5,
1.10.6.6, 1.10.6.7, 1.10.6.8, 1.10.6.9, 1.10.6.10, 1.10.7.1, 1.10.7.2, 1.10.7.3, 1.10.7.4, 1.10.7.5,
1.10.7.6, 1.10.7.7, 1.10.7.8, 1.10.7.9, 1.10.7.10, 1.10.8.1, 1.10.8.2, 1.10.8.3, 1.10.8.4, 1.10.8.5,
1.10.8.6, 1.10.8.7, 1.10.8.8, 1.10.8.9, 1.10.8.10, 1.10.9.1, 1.10.9.2, 1.10.9.3, 1.10.9.4, 1.10.9.5,
1.10.9.6, 1.10.9.7, 1.10.9.8, 1.10.9.9, 1.10.9.10, 1.10.10.1, 1.10.10.2, 1.10.10.3, 1.10.10.4,
1.10.10.5, 1.10.10.6, 1.10.10.7, 1.10.10.8, 1.10.10.9, 1.10.10.10, 2.1.1.1, 2.1.1.2, 2.1.1.3, 2.1.1.4,
2.1.1.5, 2.1.1.6, 2.1.1.7, 2.1.1.8, 2.1.1.9, 2.1.1.10, 2.1.2.1, 2.1.2.2, 2.1.2.3, 2.1.2.4, 2.1.2.5,
2.1.2.6, 2.1.2.7, 2.1.2.8, 2.1.2.9, 2.1.2.10, 2.1.3.1, 2.1.3.2, 2.1.3.3, 2.1.3.4, 2.1.3.5, 2.1.3.6,
2.1.3.7, 2.1.3.8, 2.1.3.9, 2.1.3.10, 2.1.4.1, 2.1.4.2, 2.1.4.3, 2.1.4.4, 2.1.4.5, 2.1.4.6, 2.1.4.7,
2.1.4.8, 2.1.4.9, 2.1.4.10, 2.1.5.1, 2.1.5.2, 2.1.5.3, 2.1.5.4, 2.1.5.5, 2.1.5.6, 2.1.5.7, 2.1.5.8,
2.1.5.9, 2.1.5.10, 2.1.6.1, 2.1.6.2, 2.1.6.3, 2.1.6.4, 2.1.6.5, 2.1.6.6, 2.1.6.7, 2.1.6.8, 2.1.6.9,
2.1.6.10, 2.1.7.1, 2.1.7.2, 2.1.7.3, 2.1.7.4, 2.1.7.5, 2.1.7.6, 2.1.7.7, 2.1.7.8, 2.1.7.9, 2.1.7.10,
2.1.8.1, 2.1.8.2, 2.1.8.3, 2.1.8.4, 2.1.8.5, 2.1.8.6, 2.1.8.7, 2.1.8.8, 2.1.8.9, 2.1.8.10, 2.1.9.1,
2.1.9.2, 2.1.9.3, 2.1.9.4, 2.1.9.5, 2.1.9.6, 2.1.9.7, 2.1.9.8, 2.1.9.9, 2.1.9.10, 2.1.10.1, 2.1.10.2,
2.1.10.3, 2.1.10.4, 2.1.10.5, 2.1.10.6, 2.1.10.7, 2.1.10.8, 2.1.10.9, 2.1.10.10, 2.2.1.1, 2.2.1.2,
2.2.1.3, 2.2.1.4, 2.2.1.5, 2.2.1.6, 2.2.1.7, 2.2.1.8, 2.2.1.9, 2.2.1.10, 2.2.2.1, 2.2.2.2, 2.2.2.3,
2.2.2.4, 2.2.2.5, 2.2.2.6, 2.2.2.7, 2.2.2.8, 2.2.2.9, 2.2.2.10, 2.2.3.1, 2.2.3.2, 2.2.3.3, 2.2.3.4,
2.2.3.5, 2.2.3.6, 2.2.3.7, 2.2.3.8, 2.2.3.9, 2.2.3.10, 2.2.4.1, 2.2.4.2, 2.2.4.3, 2.2.4.4, 2.2.4.5,
2.2.4.6, 2.2.4.7, 2.2.4.8, 2.2.4.9, 2.2.4.10, 2.2.5.1, 2.2.5.2, 2.2.5.3, 2.2.5.4, 2.2.5.5, 2.2.5.6,
2.2.5.7, 2.2.5.8, 2.2.5.9, 2.2.5.10, 2.2.6.1, 2.2.6.2, 2.2.6.3, 2.2.6.4, 2.2.6.5, 2.2.6.6, 2.2.6.7,
2.2.6.8, 2.2.6.9, 2.2.6.10, 2.2.7.1, 2.2.7.2, 2.2.7.3, 2.2.7.4, 2.2.7.5, 2.2.7.6, 2.2.7.7, 2.2.7.8,
2.2.7.9, 2.2.7.10, 2.2.8.1, 2.2.8.2, 2.2.8.3, 2.2.8.4, 2.2.8.5, 2.2.8.6, 2.2.8.7, 2.2.8.8, 2.2.8.9,
2.2.8.10, 2.2.9.1, 2.2.9.2, 2.2.9.3, 2.2.9.4, 2.2.9.5, 2.2.9.6, 2.2.9.7, 2.2.9.8, 2.2.9.9, 2.2.9.10,
2.2.10.1, 2.2.10.2, 2.2.10.3, 2.2.10.4, 2.2.10.5, 2.2.10.6, 2.2.10.7, 2.2.10.8, 2.2.10.9, 2.2.10.10,
2.3.1.1, 2.3.1.2, 2.3.1.3, 2.3.1.4, 2.3.1.5, 2.3.1.6, 2.3.1.7, 2.3.1.8, 2.3.1.9, 2.3.1.10, 2.3.2.1,
2.3.2.2, 2.3.2.3, 2.3.2.4, 2.3.2.5, 2.3.2.6, 2.3.2.7, 2.3.2.8, 2.3.2.9, 2.3.2.10, 2.3.3.1, 2.3.3.2,
2.3.3.3, 2.3.3.4, 2.3.3.5, 2.3.3.6, 2.3.3.7, 2.3.3.8, 2.3.3.9, 2.3.3.10, 2.3.4.1, 2.3.4.2, 2.3.4.3,
2.3.4.4, 2.3.4.5, 2.3.4.6, 2.3.4.7, 2.3.4.8, 2.3.4.9, 2.3.4.10, 2.3.5.1, 2.3.5.2, 2.3.5.3, 2.3.5.4,
2.3.5.5, 2.3.5.6, 2.3.5.7, 2.3.5.8, 2.3.5.9, 2.3.5.10, 2.3.6.1, 2.3.6.2, 2.3.6.3, 2.3.6.4, 2.3.6.5,
2.3.6.6, 2.3.6.7, 2.3.6.8, 2.3.6.9, 2.3.6.10, 2.3.7.1, 2.3.7.2, 2.3.7.3, 2.3.7.4, 2.3.7.5, 2.3.7.6,
2.3.7.7, 2.3.7.8, 2.3.7.9, 2.3.7.10, 2.3.8.1, 2.3.8.2, 2.3.8.3, 2.3.8.4, 2.3.8.5, 2.3.8.6, 2.3.8.7,
2.3.8.8, 2.3.8.9, 2.3.8.10, 2.3.9.1, 2.3.9.2, 2.3.9.3, 2.3.9.4, 2.3.9.5, 2.3.9.6, 2.3.9.7, 2.3.9.8,
2.3.9.9, 2.3.9.10, 2.3.10.1, 2.3.10.2, 2.3.10.3, 2.3.10.4, 2.3.10.5, 2.3.10.6, 2.3.10.7, 2.3.10.8,
2.3.10.9, 2.3.10.10, 2.4.1.1, 2.4.1.2, 2.4.1.3, 2.4.1.4, 2.4.1.5, 2.4.1.6, 2.4.1.7, 2.4.1.8, 2.4.1.9,
2.4.1.10, 2.4.2.1, 2.4.2.2, 2.4.2.3, 2.4.2.4, 2.4.2.5, 2.4.2.6, 2.4.2.7, 2.4.2.8, 2.4.2.9, 2.4.2.10,
2.4.3.1, 2.4.3.2, 2.4.3.3, 2.4.3.4, 2.4.3.5, 2.4.3.6, 2.4.3.7, 2.4.3.8, 2.4.3.9, 2.4.3.10, 2.4.4.1,
2.4.4.2, 2.4.4.3, 2.4.4.4, 2.4.4.5, 2.4.4.6, 2.4.4.7, 2.4.4.8, 2.4.4.9, 2.4.4.10, 2.4.5.1, 2.4.5.2,
2.4.5.3, 2.4.5.4, 2.4.5.5, 2.4.5.6, 2.4.5.7, 2.4.5.8, 2.4.5.9, 2.4.5.10, 2.4.6.1, 2.4.6.2, 2.4.6.3,
2.4.6.4, 2.4.6.5, 2.4.6.6, 2.4.6.7, 2.4.6.8, 2.4.6.9, 2.4.6.10, 2.4.7.1, 2.4.7.2, 2.4.7.3, 2.4.7.4,
2.4.7.5, 2.4.7.6, 2.4.7.7, 2.4.7.8, 2.4.7.9, 2.4.7.10, 2.4.8.1, 2.4.8.2, 2.4.8.3, 2.4.8.4, 2.4.8.5,
2.4.8.6, 2.4.8.7, 2.4.8.8, 2.4.8.9, 2.4.8.10, 2.4.9.1, 2.4.9.2, 2.4.9.3, 2.4.9.4, 2.4.9.5, 2.4.9.6,
2.4.9.7, 2.4.9.8, 2.4.9.9, 2.4.9.10, 2.4.10.1, 2.4.10.2, 2.4.10.3, 2.4.10.4, 2.4.10.5, 2.4.10.6,
2.4.10.7, 2.4.10.8, 2.4.10.9, 2.4.10.10, 2.5.1.1, 2.5.1.2, 2.5.1.3, 2.5.1.4, 2.5.1.5, 2.5.1.6, 2.5.1.7,
2.5.1.8, 2.5.1.9, 2.5.1.10, 2.5.2.1, 2.5.2.2, 2.5.2.3, 2.5.2.4, 2.5.2.5, 2.5.2.6, 2.5.2.7, 2.5.2.8,
2.5.2.9, 2.5.2.10, 2.5.3.1, 2.5.3.2, 2.5.3.3, 2.5.3.4, 2.5.3.5, 2.5.3.6, 2.5.3.7, 2.5.3.8, 2.5.3.9,
2.5.3.10, 2.5.4.1, 2.5.4.2, 2.5.4.3, 2.5.4.4, 2.5.4.5, 2.5.4.6, 2.5.4.7, 2.5.4.8, 2.5.4.9, 2.5.4.10,
2.5.5.1, 2.5.5.2, 2.5.5.3, 2.5.5.4, 2.5.5.5, 2.5.5.6, 2.5.5.7, 2.5.5.8, 2.5.5.9, 2.5.5.10, 2.5.6.1,
2.5.6.2, 2.5.6.3, 2.5.6.4, 2.5.6.5, 2.5.6.6, 2.5.6.7, 2.5.6.8, 2.5.6.9, 2.5.6.10, 2.5.7.1, 2.5.7.2,
2.5.7.3, 2.5.7.4, 2.5.7.5, 2.5.7.6, 2.5.7.7, 2.5.7.8, 2.5.7.9, 2.5.7.10, 2.5.8.1, 2.5.8.2, 2.5.8.3,
2.5.8.4, 2.5.8.5, 2.5.8.6, 2.5.8.7, 2.5.8.8, 2.5.8.9, 2.5.8.10, 2.5.9.1, 2.5.9.2, 2.5.9.3, 2.5.9.4,
2.5.9.5, 2.5.9.6, 2.5.9.7, 2.5.9.8, 2.5.9.9, 2.5.9.10, 2.5.10.1, 2.5.10.2, 2.5.10.3, 2.5.10.4, 2.5.10.5,
2.5.10.6, 2.5.10.7, 2.5.10.8, 2.5.10.9, 2.5.10.10, 2.6.1.1, 2.6.1.2, 2.6.1.3, 2.6.1.4, 2.6.1.5, 2.6.1.6,
2.6.1.7, 2.6.1.8, 2.6.1.9, 2.6.1.10, 2.6.2.1, 2.6.2.2, 2.6.2.3, 2.6.2.4, 2.6.2.5, 2.6.2.6, 2.6.2.7,
2.6.2.8, 2.6.2.9, 2.6.2.10, 2.6.3.1, 2.6.3.2, 2.6.3.3, 2.6.3.4, 2.6.3.5, 2.6.3.6, 2.6.3.7, 2.6.3.8,
2.6.3.9, 2.6.3.10, 2.6.4.1, 2.6.4.2, 2.6.4.3, 2.6.4.4, 2.6.4.5, 2.6.4.6, 2.6.4.7, 2.6.4.8, 2.6.4.9,
2.6.4.10, 2.6.5.1, 2.6.5.2, 2.6.5.3, 2.6.5.4, 2.6.5.5, 2.6.5.6, 2.6.5.7, 2.6.5.8, 2.6.5.9, 2.6.5.10,
2.6.6.1, 2.6.6.2, 2.6.6.3, 2.6.6.4, 2.6.6.5, 2.6.6.6, 2.6.6.7, 2.6.6.8, 2.6.6.9, 2.6.6.10, 2.6.7.1,
2.6.7.2, 2.6.7.3, 2.6.7.4, 2.6.7.5, 2.6.7.6, 2.6.7.7, 2.6.7.8, 2.6.7.9, 2.6.7.10, 2.6.8.1, 2.6.8.2,
2.6.8.3, 2.6.8.4, 2.6.8.5, 2.6.8.6, 2.6.8.7, 2.6.8.8, 2.6.8.9, 2.6.8.10, 2.6.9.1, 2.6.9.2, 2.6.9.3,
2.6.9.4, 2.6.9.5, 2.6.9.6, 2.6.9.7, 2.6.9.8, 2.6.9.9, 2.6.9.10, 2.6.10.1, 2.6.10.2, 2.6.10.3, 2.6.10.4,
2.6.10.5, 2.6.10.6, 2.6.10.7, 2.6.10.8, 2.6.10.9, 2.6.10.10, 2.7.1.1, 2.7.1.2, 2.7.1.3, 2.7.1.4,
2.7.1.5, 2.7.1.6, 2.7.1.7, 2.7.1.8, 2.7.1.9, 2.7.1.10, 2.7.2.1, 2.7.2.2, 2.7.2.3, 2.7.2.4, 2.7.2.5,
2.7.2.6, 2.7.2.7, 2.7.2.8, 2.7.2.9, 2.7.2.10, 2.7.3.1, 2.7.3.2, 2.7.3.3, 2.7.3.4, 2.7.3.5, 2.7.3.6,
2.7.3.7, 2.7.3.8, 2.7.3.9, 2.7.3.10, 2.7.4.1, 2.7.4.2, 2.7.4.3, 2.7.4.4, 2.7.4.5, 2.7.4.6, 2.7.4.7,
2.7.4.8, 2.7.4.9, 2.7.4.10, 2.7.5.1, 2.7.5.2, 2.7.5.3, 2.7.5.4, 2.7.5.5, 2.7.5.6, 2.7.5.7, 2.7.5.8,
2.7.5.9, 2.7.5.10, 2.7.6.1, 2.7.6.2, 2.7.6.3, 2.7.6.4, 2.7.6.5, 2.7.6.6, 2.7.6.7, 2.7.6.8, 2.7.6.9,
2.7.6.10, 2.7.7.1, 2.7.7.2, 2.7.7.3, 2.7.7.4, 2.7.7.5, 2.7.7.6, 2.7.7.7, 2.7.7.8, 2.7.7.9, 2.7.7.10,
2.7.8.1, 2.7.8.2, 2.7.8.3, 2.7.8.4, 2.7.8.5, 2.7.8.6, 2.7.8.7, 2.7.8.8, 2.7.8.9, 2.7.8.10, 2.7.9.1,
2.7.9.2, 2.7.9.3, 2.7.9.4, 2.7.9.5, 2.7.9.6, 2.7.9.7, 2.7.9.8, 2.7.9.9, 2.7.9.10, 2.7.10.1, 2.7.10.2,
2.7.10.3, 2.7.10.4, 2.7.10.5, 2.7.10.6, 2.7.10.7, 2.7.10.8, 2.7.10.9, 2.7.10.10, 2.8.1.1, 2.8.1.2,
2.8.1.3, 2.8.1.4, 2.8.1.5, 2.8.1.6, 2.8.1.7, 2.8.1.8, 2.8.1.9, 2.8.1.10, 2.8.2.1, 2.8.2.2, 2.8.2.3,
2.8.2.4, 2.8.2.5, 2.8.2.6, 2.8.2.7, 2.8.2.8, 2.8.2.9, 2.8.2.10, 2.8.3.1, 2.8.3.2, 2.8.3.3, 2.8.3.4,
2.8.3.5, 2.8.3.6, 2.8.3.7, 2.8.3.8, 2.8.3.9, 2.8.3.10, 2.8.4.1, 2.8.4.2, 2.8.4.3, 2.8.4.4, 2.8.4.5,
2.8.4.6, 2.8.4.7, 2.8.4.8, 2.8.4.9, 2.8.4.10, 2.8.5.1, 2.8.5.2, 2.8.5.3, 2.8.5.4, 2.8.5.5, 2.8.5.6,
2.8.5.7, 2.8.5.8, 2.8.5.9, 2.8.5.10, 2.8.6.1, 2.8.6.2, 2.8.6.3, 2.8.6.4, 2.8.6.5, 2.8.6.6, 2.8.6.7,
2.8.6.8, 2.8.6.9, 2.8.6.10, 2.8.7.1, 2.8.7.2, 2.8.7.3, 2.8.7.4, 2.8.7.5, 2.8.7.6, 2.8.7.7, 2.8.7.8,
2.8.7.9, 2.8.7.10, 2.8.8.1, 2.8.8.2, 2.8.8.3, 2.8.8.4, 2.8.8.5, 2.8.8.6, 2.8.8.7, 2.8.8.8, 2.8.8.9,
2.8.8.10, 2.8.9.1, 2.8.9.2, 2.8.9.3, 2.8.9.4, 2.8.9.5, 2.8.9.6, 2.8.9.7, 2.8.9.8, 2.8.9.9, 2.8.9.10,
2.8.10.1, 2.8.10.2, 2.8.10.3, 2.8.10.4, 2.8.10.5, 2.8.10.6, 2.8.10.7, 2.8.10.8, 2.8.10.9, 2.8.10.10,
2.9.1.1, 2.9.1.2, 2.9.1.3, 2.9.1.4, 2.9.1.5, 2.9.1.6, 2.9.1.7, 2.9.1.8, 2.9.1.9, 2.9.1.10, 2.9.2.1,
2.9.2.2, 2.9.2.3, 2.9.2.4, 2.9.2.5, 2.9.2.6, 2.9.2.7, 2.9.2.8, 2.9.2.9, 2.9.2.10, 2.9.3.1, 2.9.3.2,
2.9.3.3, 2.9.3.4, 2.9.3.5, 2.9.3.6, 2.9.3.7, 2.9.3.8, 2.9.3.9, 2.9.3.10, 2.9.4.1, 2.9.4.2, 2.9.4.3,
2.9.4.4, 2.9.4.5, 2.9.4.6, 2.9.4.7, 2.9.4.8, 2.9.4.9, 2.9.4.10, 2.9.5.1, 2.9.5.2, 2.9.5.3, 2.9.5.4,
2.9.5.5, 2.9.5.6, 2.9.5.7, 2.9.5.8, 2.9.5.9, 2.9.5.10, 2.9.6.1, 2.9.6.2, 2.9.6.3, 2.9.6.4, 2.9.6.5,
2.9.6.6, 2.9.6.7, 2.9.6.8, 2.9.6.9, 2.9.6.10, 2.9.7.1, 2.9.7.2, 2.9.7.3, 2.9.7.4, 2.9.7.5, 2.9.7.6,
2.9.7.7, 2.9.7.8, 2.9.7.9, 2.9.7.10, 2.9.8.1, 2.9.8.2, 2.9.8.3, 2.9.8.4, 2.9.8.5, 2.9.8.6, 2.9.8.7,
2.9.8.8, 2.9.8.9, 2.9.8.10, 2.9.9.1, 2.9.9.2, 2.9.9.3, 2.9.9.4, 2.9.9.5, 2.9.9.6, 2.9.9.7, 2.9.9.8,
2.9.9.9, 2.9.9.10, 2.9.10.1, 2.9.10.2, 2.9.10.3, 2.9.10.4, 2.9.10.5, 2.9.10.6, 2.9.10.7, 2.9.10.8,
2.9.10.9, 2.9.10.10, 2.10.1.1, 2.10.1.2, 2.10.1.3, 2.10.1.4, 2.10.1.5, 2.10.1.6, 2.10.1.7, 2.10.1.8,
2.10.1.9, 2.10.1.10, 2.10.2.1, 2.10.2.2, 2.10.2.3, 2.10.2.4, 2.10.2.5, 2.10.2.6, 2.10.2.7, 2.10.2.8,
2.10.2.9, 2.10.2.10, 2.10.3.1, 2.10.3.2, 2.10.3.3, 2.10.3.4, 2.10.3.5, 2.10.3.6, 2.10.3.7, 2.10.3.8,
2.10.3.9, 2.10.3.10, 2.10.4.1, 2.10.4.2, 2.10.4.3, 2.10.4.4, 2.10.4.5, 2.10.4.6, 2.10.4.7, 2.10.4.8,
2.10.4.9, 2.10.4.10, 2.10.5.1, 2.10.5.2, 2.10.5.3, 2.10.5.4, 2.10.5.5, 2.10.5.6, 2.10.5.7, 2.10.5.8,
2.10.5.9, 2.10.5.10, 2.10.6.1, 2.10.6.2, 2.10.6.3, 2.10.6.4, 2.10.6.5, 2.10.6.6, 2.10.6.7, 2.10.6.8,
2.10.6.9, 2.10.6.10, 2.10.7.1, 2.10.7.2, 2.10.7.3, 2.10.7.4, 2.10.7.5, 2.10.7.6, 2.10.7.7, 2.10.7.8,
2.10.7.9, 2.10.7.10, 2.10.8.1, 2.10.8.2, 2.10.8.3, 2.10.8.4, 2.10.8.5, 2.10.8.6, 2.10.8.7, 2.10.8.8,
2.10.8.9, 2.10.8.10, 2.10.9.1, 2.10.9.2, 2.10.9.3, 2.10.9.4, 2.10.9.5, 2.10.9.6, 2.10.9.7, 2.10.9.8,
2.10.9.9, 2.10.9.10, 2.10.10.1, 2.10.10.2, 2.10.10.3, 2.10.10.4, 2.10.10.5, 2.10.10.6, 2.10.10.7,
2.10.10.8, 2.10.10.9, 2.10.10.10, 3.1.1.1, 3.1.1.2, 3.1.1.3, 3.1.1.4, 3.1.1.5, 3.1.1.6, 3.1.1.7,
3.1.1.8, 3.1.1.9, 3.1.1.10, 3.1.2.1, 3.1.2.2, 3.1.2.3, 3.1.2.4, 3.1.2.5, 3.1.2.6, 3.1.2.7, 3.1.2.8,
3.1.2.9, 3.1.2.10, 3.1.3.1, 3.1.3.2, 3.1.3.3, 3.1.3.4, 3.1.3.5, 3.1.3.6, 3.1.3.7, 3.1.3.8, 3.1.3.9,
3.1.3.10, 3.1.4.1, 3.1.4.2, 3.1.4.3, 3.1.4.4, 3.1.4.5, 3.1.4.6, 3.1.4.7, 3.1.4.8, 3.1.4.9, 3.1.4.10,
3.1.5.1, 3.1.5.2, 3.1.5.3, 3.1.5.4, 3.1.5.5, 3.1.5.6, 3.1.5.7, 3.1.5.8, 3.1.5.9, 3.1.5.10, 3.1.6.1,
3.1.6.2, 3.1.6.3, 3.1.6.4, 3.1.6.5, 3.1.6.6, 3.1.6.7, 3.1.6.8, 3.1.6.9, 3.1.6.10, 3.1.7.1, 3.1.7.2,
3.1.7.3, 3.1.7.4, 3.1.7.5, 3.1.7.6, 3.1.7.7, 3.1.7.8, 3.1.7.9, 3.1.7.10, 3.1.8.1, 3.1.8.2, 3.1.8.3,
3.1.8.4, 3.1.8.5, 3.1.8.6, 3.1.8.7, 3.1.8.8, 3.1.8.9, 3.1.8.10, 3.1.9.1, 3.1.9.2, 3.1.9.3, 3.1.9.4,
3.1.9.5, 3.1.9.6, 3.1.9.7, 3.1.9.8, 3.1.9.9, 3.1.9.10, 3.1.10.1, 3.1.10.2, 3.1.10.3, 3.1.10.4, 3.1.10.5,
3.1.10.6, 3.1.10.7, 3.1.10.8, 3.1.10.9, 3.1.10.10, 3.2.1.1, 3.2.1.2, 3.2.1.3, 3.2.1.4, 3.2.1.5, 3.2.1.6,
3.2.1.7, 3.2.1.8, 3.2.1.9, 3.2.1.10, 3.2.2.1, 3.2.2.2, 3.2.2.3, 3.2.2.4, 3.2.2.5, 3.2.2.6, 3.2.2.7,
3.2.2.8, 3.2.2.9, 3.2.2.10, 3.2.3.1, 3.2.3.2, 3.2.3.3, 3.2.3.4, 3.2.3.5, 3.2.3.6, 3.2.3.7, 3.2.3.8,
3.2.3.9, 3.2.3.10, 3.2.4.1, 3.2.4.2, 3.2.4.3, 3.2.4.4, 3.2.4.5, 3.2.4.6, 3.2.4.7, 3.2.4.8, 3.2.4.9,
3.2.4.10, 3.2.5.1, 3.2.5.2, 3.2.5.3, 3.2.5.4, 3.2.5.5, 3.2.5.6, 3.2.5.7, 3.2.5.8, 3.2.5.9, 3.2.5.10,
3.2.6.1, 3.2.6.2, 3.2.6.3, 3.2.6.4, 3.2.6.5, 3.2.6.6, 3.2.6.7, 3.2.6.8, 3.2.6.9, 3.2.6.10, 3.2.7.1,
3.2.7.2, 3.2.7.3, 3.2.7.4, 3.2.7.5, 3.2.7.6, 3.2.7.7, 3.2.7.8, 3.2.7.9, 3.2.7.10, 3.2.8.1, 3.2.8.2,
3.2.8.3, 3.2.8.4, 3.2.8.5, 3.2.8.6, 3.2.8.7, 3.2.8.8, 3.2.8.9, 3.2.8.10, 3.2.9.1, 3.2.9.2, 3.2.9.3,
3.2.9.4, 3.2.9.5, 3.2.9.6, 3.2.9.7, 3.2.9.8, 3.2.9.9, 3.2.9.10, 3.2.10.1, 3.2.10.2, 3.2.10.3, 3.2.10.4,
3.2.10.5, 3.2.10.6, 3.2.10.7, 3.2.10.8, 3.2.10.9, 3.2.10.10, 3.3.1.1, 3.3.1.2, 3.3.1.3, 3.3.1.4,
3.3.1.5, 3.3.1.6, 3.3.1.7, 3.3.1.8, 3.3.1.9, 3.3.1.10, 3.3.2.1, 3.3.2.2, 3.3.2.3, 3.3.2.4, 3.3.2.5,
3.3.2.6, 3.3.2.7, 3.3.2.8, 3.3.2.9, 3.3.2.10, 3.3.3.1, 3.3.3.2, 3.3.3.3, 3.3.3.4, 3.3.3.5, 3.3.3.6,
3.3.3.7, 3.3.3.8, 3.3.3.9, 3.3.3.10, 3.3.4.1, 3.3.4.2, 3.3.4.3, 3.3.4.4, 3.3.4.5, 3.3.4.6, 3.3.4.7,
3.3.4.8, 3.3.4.9, 3.3.4.10, 3.3.5.1, 3.3.5.2, 3.3.5.3, 3.3.5.4, 3.3.5.5, 3.3.5.6, 3.3.5.7, 3.3.5.8,
3.3.5.9, 3.3.5.10, 3.3.6.1, 3.3.6.2, 3.3.6.3, 3.3.6.4, 3.3.6.5, 3.3.6.6, 3.3.6.7, 3.3.6.8, 3.3.6.9,
3.3.6.10, 3.3.7.1, 3.3.7.2, 3.3.7.3, 3.3.7.4, 3.3.7.5, 3.3.7.6, 3.3.7.7, 3.3.7.8, 3.3.7.9, 3.3.7.10,
3.3.8.1, 3.3.8.2, 3.3.8.3, 3.3.8.4, 3.3.8.5, 3.3.8.6, 3.3.8.7, 3.3.8.8, 3.3.8.9, 3.3.8.10, 3.3.9.1,
3.3.9.2, 3.3.9.3, 3.3.9.4, 3.3.9.5, 3.3.9.6, 3.3.9.7, 3.3.9.8, 3.3.9.9, 3.3.9.10, 3.3.10.1, 3.3.10.2,
3.3.10.3, 3.3.10.4, 3.3.10.5, 3.3.10.6, 3.3.10.7, 3.3.10.8, 3.3.10.9, 3.3.10.10, 3.4.1.1, 3.4.1.2,
3.4.1.3, 3.4.1.4, 3.4.1.5, 3.4.1.6, 3.4.1.7, 3.4.1.8, 3.4.1.9, 3.4.1.10, 3.4.2.1, 3.4.2.2, 3.4.2.3,
3.4.2.4, 3.4.2.5, 3.4.2.6, 3.4.2.7, 3.4.2.8, 3.4.2.9, 3.4.2.10, 3.4.3.1, 3.4.3.2, 3.4.3.3, 3.4.3.4,
3.4.3.5, 3.4.3.6, 3.4.3.7, 3.4.3.8, 3.4.3.9, 3.4.3.10, 3.4.4.1, 3.4.4.2, 3.4.4.3, 3.4.4.4, 3.4.4.5,
3.4.4.6, 3.4.4.7, 3.4.4.8, 3.4.4.9, 3.4.4.10, 3.4.5.1, 3.4.5.2, 3.4.5.3, 3.4.5.4, 3.4.5.5, 3.4.5.6,
3.4.5.7, 3.4.5.8, 3.4.5.9, 3.4.5.10, 3.4.6.1, 3.4.6.2, 3.4.6.3, 3.4.6.4, 3.4.6.5, 3.4.6.6, 3.4.6.7,
3.4.6.8, 3.4.6.9, 3.4.6.10, 3.4.7.1, 3.4.7.2, 3.4.7.3, 3.4.7.4, 3.4.7.5, 3.4.7.6, 3.4.7.7, 3.4.7.8,
3.4.7.9, 3.4.7.10, 3.4.8.1, 3.4.8.2, 3.4.8.3, 3.4.8.4, 3.4.8.5, 3.4.8.6, 3.4.8.7, 3.4.8.8, 3.4.8.9,
3.4.8.10, 3.4.9.1, 3.4.9.2, 3.4.9.3, 3.4.9.4, 3.4.9.5, 3.4.9.6, 3.4.9.7, 3.4.9.8, 3.4.9.9, 3.4.9.10,
3.4.10.1, 3.4.10.2, 3.4.10.3, 3.4.10.4, 3.4.10.5, 3.4.10.6, 3.4.10.7, 3.4.10.8, 3.4.10.9, 3.4.10.10,
3.5.1.1, 3.5.1.2, 3.5.1.3, 3.5.1.4, 3.5.1.5, 3.5.1.6, 3.5.1.7, 3.5.1.8, 3.5.1.9, 3.5.1.10, 3.5.2.1,
3.5.2.2, 3.5.2.3, 3.5.2.4, 3.5.2.5, 3.5.2.6, 3.5.2.7, 3.5.2.8, 3.5.2.9, 3.5.2.10, 3.5.3.1, 3.5.3.2,
3.5.3.3, 3.5.3.4, 3.5.3.5, 3.5.3.6, 3.5.3.7, 3.5.3.8, 3.5.3.9, 3.5.3.10, 3.5.4.1, 3.5.4.2, 3.5.4.3,
3.5.4.4, 3.5.4.5, 3.5.4.6, 3.5.4.7, 3.5.4.8, 3.5.4.9, 3.5.4.10, 3.5.5.1, 3.5.5.2, 3.5.5.3, 3.5.5.4,
3.5.5.5, 3.5.5.6, 3.5.5.7, 3.5.5.8, 3.5.5.9, 3.5.5.10, 3.5.6.1, 3.5.6.2, 3.5.6.3, 3.5.6.4, 3.5.6.5,
3.5.6.6, 3.5.6.7, 3.5.6.8, 3.5.6.9, 3.5.6.10, 3.5.7.1, 3.5.7.2, 3.5.7.3, 3.5.7.4, 3.5.7.5, 3.5.7.6,
3.5.7.7, 3.5.7.8, 3.5.7.9, 3.5.7.10, 3.5.8.1, 3.5.8.2, 3.5.8.3, 3.5.8.4, 3.5.8.5, 3.5.8.6, 3.5.8.7,
3.5.8.8, 3.5.8.9, 3.5.8.10, 3.5.9.1, 3.5.9.2, 3.5.9.3, 3.5.9.4, 3.5.9.5, 3.5.9.6, 3.5.9.7, 3.5.9.8,
3.5.9.9, 3.5.9.10, 3.5.10.1, 3.5.10.2, 3.5.10.3, 3.5.10.4, 3.5.10.5, 3.5.10.6, 3.5.10.7, 3.5.10.8,
3.5.10.9, 3.5.10.10, 3.6.1.1, 3.6.1.2, 3.6.1.3, 3.6.1.4, 3.6.1.5, 3.6.1.6, 3.6.1.7, 3.6.1.8, 3.6.1.9,
3.6.1.10, 3.6.2.1, 3.6.2.2, 3.6.2.3, 3.6.2.4, 3.6.2.5, 3.6.2.6, 3.6.2.7, 3.6.2.8, 3.6.2.9, 3.6.2.10,
3.6.3.1, 3.6.3.2, 3.6.3.3, 3.6.3.4, 3.6.3.5, 3.6.3.6, 3.6.3.7, 3.6.3.8, 3.6.3.9, 3.6.3.10, 3.6.4.1,
3.6.4.2, 3.6.4.3, 3.6.4.4, 3.6.4.5, 3.6.4.6, 3.6.4.7, 3.6.4.8, 3.6.4.9, 3.6.4.10, 3.6.5.1, 3.6.5.2,
3.6.5.3, 3.6.5.4, 3.6.5.5, 3.6.5.6, 3.6.5.7, 3.6.5.8, 3.6.5.9, 3.6.5.10, 3.6.6.1, 3.6.6.2, 3.6.6.3,
3.6.6.4, 3.6.6.5, 3.6.6.6, 3.6.6.7, 3.6.6.8, 3.6.6.9, 3.6.6.10, 3.6.7.1, 3.6.7.2, 3.6.7.3, 3.6.7.4,
3.6.7.5, 3.6.7.6, 3.6.7.7, 3.6.7.8, 3.6.7.9, 3.6.7.10, 3.6.8.1, 3.6.8.2, 3.6.8.3, 3.6.8.4, 3.6.8.5,
3.6.8.6, 3.6.8.7, 3.6.8.8, 3.6.8.9, 3.6.8.10, 3.6.9.1, 3.6.9.2, 3.6.9.3, 3.6.9.4, 3.6.9.5, 3.6.9.6,
3.6.9.7, 3.6.9.8, 3.6.9.9, 3.6.9.10, 3.6.10.1, 3.6.10.2, 3.6.10.3, 3.6.10.4, 3.6.10.5, 3.6.10.6,
3.6.10.7, 3.6.10.8, 3.6.10.9, 3.6.10.10, 3.7.1.1, 3.7.1.2, 3.7.1.3, 3.7.1.4, 3.7.1.5, 3.7.1.6, 3.7.1.7,
3.7.1.8, 3.7.1.9, 3.7.1.10, 3.7.2.1, 3.7.2.2, 3.7.2.3, 3.7.2.4, 3.7.2.5, 3.7.2.6, 3.7.2.7, 3.7.2.8,
3.7.2.9, 3.7.2.10, 3.7.3.1, 3.7.3.2, 3.7.3.3, 3.7.3.4, 3.7.3.5, 3.7.3.6, 3.7.3.7, 3.7.3.8, 3.7.3.9,
3.7.3.10, 3.7.4.1, 3.7.4.2, 3.7.4.3, 3.7.4.4, 3.7.4.5, 3.7.4.6, 3.7.4.7, 3.7.4.8, 3.7.4.9, 3.7.4.10,
3.7.5.1, 3.7.5.2, 3.7.5.3, 3.7.5.4, 3.7.5.5, 3.7.5.6, 3.7.5.7, 3.7.5.8, 3.7.5.9, 3.7.5.10, 3.7.6.1,
3.7.6.2, 3.7.6.3, 3.7.6.4, 3.7.6.5, 3.7.6.6, 3.7.6.7, 3.7.6.8, 3.7.6.9, 3.7.6.10, 3.7.7.1, 3.7.7.2,
3.7.7.3, 3.7.7.4, 3.7.7.5, 3.7.7.6, 3.7.7.7, 3.7.7.8, 3.7.7.9, 3.7.7.10, 3.7.8.1, 3.7.8.2, 3.7.8.3,
3.7.8.4, 3.7.8.5, 3.7.8.6, 3.7.8.7, 3.7.8.8, 3.7.8.9, 3.7.8.10, 3.7.9.1, 3.7.9.2, 3.7.9.3, 3.7.9.4,
3.7.9.5, 3.7.9.6, 3.7.9.7, 3.7.9.8, 3.7.9.9, 3.7.9.10, 3.7.10.1, 3.7.10.2, 3.7.10.3, 3.7.10.4, 3.7.10.5,
3.7.10.6, 3.7.10.7, 3.7.10.8, 3.7.10.9, 3.7.10.10, 3.8.1.1, 3.8.1.2, 3.8.1.3, 3.8.1.4, 3.8.1.5, 3.8.1.6,
3.8.1.7, 3.8.1.8, 3.8.1.9, 3.8.1.10, 3.8.2.1, 3.8.2.2, 3.8.2.3, 3.8.2.4, 3.8.2.5, 3.8.2.6, 3.8.2.7,
3.8.2.8, 3.8.2.9, 3.8.2.10, 3.8.3.1, 3.8.3.2, 3.8.3.3, 3.8.3.4, 3.8.3.5, 3.8.3.6, 3.8.3.7, 3.8.3.8,
3.8.3.9, 3.8.3.10, 3.8.4.1, 3.8.4.2, 3.8.4.3, 3.8.4.4, 3.8.4.5, 3.8.4.6, 3.8.4.7, 3.8.4.8, 3.8.4.9,
3.8.4.10, 3.8.5.1, 3.8.5.2, 3.8.5.3, 3.8.5.4, 3.8.5.5, 3.8.5.6, 3.8.5.7, 3.8.5.8, 3.8.5.9, 3.8.5.10,
3.8.6.1, 3.8.6.2, 3.8.6.3, 3.8.6.4, 3.8.6.5, 3.8.6.6, 3.8.6.7, 3.8.6.8, 3.8.6.9, 3.8.6.10, 3.8.7.1,
3.8.7.2, 3.8.7.3, 3.8.7.4, 3.8.7.5, 3.8.7.6, 3.8.7.7, 3.8.7.8, 3.8.7.9, 3.8.7.10, 3.8.8.1, 3.8.8.2,
3.8.8.3, 3.8.8.4, 3.8.8.5, 3.8.8.6, 3.8.8.7, 3.8.8.8, 3.8.8.9, 3.8.8.10, 3.8.9.1, 3.8.9.2, 3.8.9.3,
3.8.9.4, 3.8.9.5, 3.8.9.6, 3.8.9.7, 3.8.9.8, 3.8.9.9, 3.8.9.10, 3.8.10.1, 3.8.10.2, 3.8.10.3, 3.8.10.4,
3.8.10.5, 3.8.10.6, 3.8.10.7, 3.8.10.8, 3.8.10.9, 3.8.10.10, 3.9.1.1, 3.9.1.2, 3.9.1.3, 3.9.1.4,
3.9.1.5, 3.9.1.6, 3.9.1.7, 3.9.1.8, 3.9.1.9, 3.9.1.10, 3.9.2.1, 3.9.2.2, 3.9.2.3, 3.9.2.4, 3.9.2.5,
3.9.2.6, 3.9.2.7, 3.9.2.8, 3.9.2.9, 3.9.2.10, 3.9.3.1, 3.9.3.2, 3.9.3.3, 3.9.3.4, 3.9.3.5, 3.9.3.6,
3.9.3.7, 3.9.3.8, 3.9.3.9, 3.9.3.10, 3.9.4.1, 3.9.4.2, 3.9.4.3, 3.9.4.4, 3.9.4.5, 3.9.4.6, 3.9.4.7,
3.9.4.8, 3.9.4.9, 3.9.4.10, 3.9.5.1, 3.9.5.2, 3.9.5.3, 3.9.5.4, 3.9.5.5, 3.9.5.6, 3.9.5.7, 3.9.5.8,
3.9.5.9, 3.9.5.10, 3.9.6.1, 3.9.6.2, 3.9.6.3, 3.9.6.4, 3.9.6.5, 3.9.6.6, 3.9.6.7, 3.9.6.8, 3.9.6.9,
3.9.6.10, 3.9.7.1, 3.9.7.2, 3.9.7.3, 3.9.7.4, 3.9.7.5, 3.9.7.6, 3.9.7.7, 3.9.7.8, 3.9.7.9, 3.9.7.10,
3.9.8.1, 3.9.8.2, 3.9.8.3, 3.9.8.4, 3.9.8.5, 3.9.8.6, 3.9.8.7, 3.9.8.8, 3.9.8.9, 3.9.8.10, 3.9.9.1,
3.9.9.2, 3.9.9.3, 3.9.9.4, 3.9.9.5, 3.9.9.6, 3.9.9.7, 3.9.9.8, 3.9.9.9, 3.9.9.10, 3.9.10.1, 3.9.10.2,
3.9.10.3, 3.9.10.4, 3.9.10.5, 3.9.10.6, 3.9.10.7, 3.9.10.8, 3.9.10.9, 3.9.10.10, 3.10.1.1, 3.10.1.2,
3.10.1.3, 3.10.1.4, 3.10.1.5, 3.10.1.6, 3.10.1.7, 3.10.1.8, 3.10.1.9, 3.10.1.10, 3.10.2.1, 3.10.2.2,
3.10.2.3, 3.10.2.4, 3.10.2.5, 3.10.2.6, 3.10.2.7, 3.10.2.8, 3.10.2.9, 3.10.2.10, 3.10.3.1, 3.10.3.2,
3.10.3.3, 3.10.3.4, 3.10.3.5, 3.10.3.6, 3.10.3.7, 3.10.3.8, 3.10.3.9, 3.10.3.10, 3.10.4.1, 3.10.4.2,
3.10.4.3, 3.10.4.4, 3.10.4.5, 3.10.4.6, 3.10.4.7, 3.10.4.8, 3.10.4.9, 3.10.4.10, 3.10.5.1, 3.10.5.2,
3.10.5.3, 3.10.5.4, 3.10.5.5, 3.10.5.6, 3.10.5.7, 3.10.5.8, 3.10.5.9, 3.10.5.10, 3.10.6.1, 3.10.6.2,
3.10.6.3, 3.10.6.4, 3.10.6.5, 3.10.6.6, 3.10.6.7, 3.10.6.8, 3.10.6.9, 3.10.6.10, 3.10.7.1, 3.10.7.2,
3.10.7.3, 3.10.7.4, 3.10.7.5, 3.10.7.6, 3.10.7.7, 3.10.7.8, 3.10.7.9, 3.10.7.10, 3.10.8.1, 3.10.8.2,
3.10.8.3, 3.10.8.4, 3.10.8.5, 3.10.8.6, 3.10.8.7, 3.10.8.8, 3.10.8.9, 3.10.8.10, 3.10.9.1, 3.10.9.2,
3.10.9.3, 3.10.9.4, 3.10.9.5, 3.10.9.6, 3.10.9.7, 3.10.9.8, 3.10.9.9, 3.10.9.10, 3.10.10.1, 3.10.10.2,
3.10.10.3, 3.10.10.4, 3.10.10.5, 3.10.10.6, 3.10.10.7, 3.10.10.8, 3.10.10.9, 3.10.10.10, 4.1.1.1,
4.1.1.2, 4.1.1.3, 4.1.1.4, 4.1.1.5, 4.1.1.6, 4.1.1.7, 4.1.1.8, 4.1.1.9, 4.1.1.10, 4.1.2.1, 4.1.2.2,
4.1.2.3, 4.1.2.4, 4.1.2.5, 4.1.2.6, 4.1.2.7, 4.1.2.8, 4.1.2.9, 4.1.2.10, 4.1.3.1, 4.1.3.2, 4.1.3.3,
4.1.3.4, 4.1.3.5, 4.1.3.6, 4.1.3.7, 4.1.3.8, 4.1.3.9, 4.1.3.10, 4.1.4.1, 4.1.4.2, 4.1.4.3, 4.1.4.4,
4.1.4.5, 4.1.4.6, 4.1.4.7, 4.1.4.8, 4.1.4.9, 4.1.4.10, 4.1.5.1, 4.1.5.2, 4.1.5.3, 4.1.5.4, 4.1.5.5,
4.1.5.6, 4.1.5.7, 4.1.5.8, 4.1.5.9, 4.1.5.10, 4.1.6.1, 4.1.6.2, 4.1.6.3, 4.1.6.4, 4.1.6.5, 4.1.6.6,
4.1.6.7, 4.1.6.8, 4.1.6.9, 4.1.6.10, 4.1.7.1, 4.1.7.2, 4.1.7.3, 4.1.7.4, 4.1.7.5, 4.1.7.6, 4.1.7.7,
4.1.7.8, 4.1.7.9, 4.1.7.10, 4.1.8.1, 4.1.8.2, 4.1.8.3, 4.1.8.4, 4.1.8.5, 4.1.8.6, 4.1.8.7, 4.1.8.8,
4.1.8.9, 4.1.8.10, 4.1.9.1, 4.1.9.2, 4.1.9.3, 4.1.9.4, 4.1.9.5, 4.1.9.6, 4.1.9.7, 4.1.9.8, 4.1.9.9,
4.1.9.10, 4.1.10.1, 4.1.10.2, 4.1.10.3, 4.1.10.4, 4.1.10.5, 4.1.10.6, 4.1.10.7, 4.1.10.8, 4.1.10.9,
4.1.10.10, 4.2.1.1, 4.2.1.2, 4.2.1.3, 4.2.1.4, 4.2.1.5, 4.2.1.6, 4.2.1.7, 4.2.1.8, 4.2.1.9, 4.2.1.10,
4.2.2.1, 4.2.2.2, 4.2.2.3, 4.2.2.4, 4.2.2.5, 4.2.2.6, 4.2.2.7, 4.2.2.8, 4.2.2.9, 4.2.2.10, 4.2.3.1,
4.2.3.2, 4.2.3.3, 4.2.3.4, 4.2.3.5, 4.2.3.6, 4.2.3.7, 4.2.3.8, 4.2.3.9, 4.2.3.10, 4.2.4.1, 4.2.4.2,
4.2.4.3, 4.2.4.4, 4.2.4.5, 4.2.4.6, 4.2.4.7, 4.2.4.8, 4.2.4.9, 4.2.4.10, 4.2.5.1, 4.2.5.2, 4.2.5.3,
4.2.5.4, 4.2.5.5, 4.2.5.6, 4.2.5.7, 4.2.5.8, 4.2.5.9, 4.2.5.10, 4.2.6.1, 4.2.6.2, 4.2.6.3, 4.2.6.4,
4.2.6.5, 4.2.6.6, 4.2.6.7, 4.2.6.8, 4.2.6.9, 4.2.6.10, 4.2.7.1, 4.2.7.2, 4.2.7.3, 4.2.7.4, 4.2.7.5,
4.2.7.6, 4.2.7.7, 4.2.7.8, 4.2.7.9, 4.2.7.10, 4.2.8.1, 4.2.8.2, 4.2.8.3, 4.2.8.4, 4.2.8.5, 4.2.8.6,
4.2.8.7, 4.2.8.8, 4.2.8.9, 4.2.8.10, 4.2.9.1, 4.2.9.2, 4.2.9.3, 4.2.9.4, 4.2.9.5, 4.2.9.6, 4.2.9.7,
4.2.9.8, 4.2.9.9, 4.2.9.10, 4.2.10.1, 4.2.10.2, 4.2.10.3, 4.2.10.4, 4.2.10.5, 4.2.10.6, 4.2.10.7,
4.2.10.8, 4.2.10.9, 4.2.10.10, 4.3.1.1, 4.3.1.2, 4.3.1.3, 4.3.1.4, 4.3.1.5, 4.3.1.6, 4.3.1.7, 4.3.1.8,
4.3.1.9, 4.3.1.10, 4.3.2.1, 4.3.2.2, 4.3.2.3, 4.3.2.4, 4.3.2.5, 4.3.2.6, 4.3.2.7, 4.3.2.8, 4.3.2.9,
4.3.2.10, 4.3.3.1, 4.3.3.2, 4.3.3.3, 4.3.3.4, 4.3.3.5, 4.3.3.6, 4.3.3.7, 4.3.3.8, 4.3.3.9, 4.3.3.10,
4.3.4.1, 4.3.4.2, 4.3.4.3, 4.3.4.4, 4.3.4.5, 4.3.4.6, 4.3.4.7, 4.3.4.8, 4.3.4.9, 4.3.4.10, 4.3.5.1,
4.3.5.2, 4.3.5.3, 4.3.5.4, 4.3.5.5, 4.3.5.6, 4.3.5.7, 4.3.5.8, 4.3.5.9, 4.3.5.10, 4.3.6.1, 4.3.6.2,
4.3.6.3, 4.3.6.4, 4.3.6.5, 4.3.6.6, 4.3.6.7, 4.3.6.8, 4.3.6.9, 4.3.6.10, 4.3.7.1, 4.3.7.2, 4.3.7.3,
4.3.7.4, 4.3.7.5, 4.3.7.6, 4.3.7.7, 4.3.7.8, 4.3.7.9, 4.3.7.10, 4.3.8.1, 4.3.8.2, 4.3.8.3, 4.3.8.4,
4.3.8.5, 4.3.8.6, 4.3.8.7, 4.3.8.8, 4.3.8.9, 4.3.8.10, 4.3.9.1, 4.3.9.2, 4.3.9.3, 4.3.9.4, 4.3.9.5,
4.3.9.6, 4.3.9.7, 4.3.9.8, 4.3.9.9, 4.3.9.10, 4.3.10.1, 4.3.10.2, 4.3.10.3, 4.3.10.4, 4.3.10.5,
4.3.10.6, 4.3.10.7, 4.3.10.8, 4.3.10.9, 4.3.10.10, 4.4.1.1, 4.4.1.2, 4.4.1.3, 4.4.1.4, 4.4.1.5, 4.4.1.6,
4.4.1.7, 4.4.1.8, 4.4.1.9, 4.4.1.10, 4.4.2.1, 4.4.2.2, 4.4.2.3, 4.4.2.4, 4.4.2.5, 4.4.2.6, 4.4.2.7,
4.4.2.8, 4.4.2.9, 4.4.2.10, 4.4.3.1, 4.4.3.2, 4.4.3.3, 4.4.3.4, 4.4.3.5, 4.4.3.6, 4.4.3.7, 4.4.3.8,
4.4.3.9, 4.4.3.10, 4.4.4.1, 4.4.4.2, 4.4.4.3, 4.4.4.4, 4.4.4.5, 4.4.4.6, 4.4.4.7, 4.4.4.8, 4.4.4.9,
4.4.4.10, 4.4.5.1, 4.4.5.2, 4.4.5.3, 4.4.5.4, 4.4.5.5, 4.4.5.6, 4.4.5.7, 4.4.5.8, 4.4.5.9, 4.4.5.10,
4.4.6.1, 4.4.6.2, 4.4.6.3, 4.4.6.4, 4.4.6.5, 4.4.6.6, 4.4.6.7, 4.4.6.8, 4.4.6.9, 4.4.6.10, 4.4.7.1,
4.4.7.2, 4.4.7.3, 4.4.7.4, 4.4.7.5, 4.4.7.6, 4.4.7.7, 4.4.7.8, 4.4.7.9, 4.4.7.10, 4.4.8.1, 4.4.8.2,
4.4.8.3, 4.4.8.4, 4.4.8.5, 4.4.8.6, 4.4.8.7, 4.4.8.8, 4.4.8.9, 4.4.8.10, 4.4.9.1, 4.4.9.2, 4.4.9.3,
4.4.9.4, 4.4.9.5, 4.4.9.6, 4.4.9.7, 4.4.9.8, 4.4.9.9, 4.4.9.10, 4.4.10.1, 4.4.10.2, 4.4.10.3, 4.4.10.4,
4.4.10.5, 4.4.10.6, 4.4.10.7, 4.4.10.8, 4.4.10.9, 4.4.10.10, 4.5.1.1, 4.5.1.2, 4.5.1.3, 4.5.1.4,
4.5.1.5, 4.5.1.6, 4.5.1.7, 4.5.1.8, 4.5.1.9, 4.5.1.10, 4.5.2.1, 4.5.2.2, 4.5.2.3, 4.5.2.4, 4.5.2.5,
4.5.2.6, 4.5.2.7, 4.5.2.8, 4.5.2.9, 4.5.2.10, 4.5.3.1, 4.5.3.2, 4.5.3.3, 4.5.3.4, 4.5.3.5, 4.5.3.6,
4.5.3.7, 4.5.3.8, 4.5.3.9, 4.5.3.10, 4.5.4.1, 4.5.4.2, 4.5.4.3, 4.5.4.4, 4.5.4.5, 4.5.4.6, 4.5.4.7,
4.5.4.8, 4.5.4.9, 4.5.4.10, 4.5.5.1, 4.5.5.2, 4.5.5.3, 4.5.5.4, 4.5.5.5, 4.5.5.6, 4.5.5.7, 4.5.5.8,
4.5.5.9, 4.5.5.10, 4.5.6.1, 4.5.6.2, 4.5.6.3, 4.5.6.4, 4.5.6.5, 4.5.6.6, 4.5.6.7, 4.5.6.8, 4.5.6.9,
4.5.6.10, 4.5.7.1, 4.5.7.2, 4.5.7.3, 4.5.7.4, 4.5.7.5, 4.5.7.6, 4.5.7.7, 4.5.7.8, 4.5.7.9, 4.5.7.10,
4.5.8.1, 4.5.8.2, 4.5.8.3, 4.5.8.4, 4.5.8.5, 4.5.8.6, 4.5.8.7, 4.5.8.8, 4.5.8.9, 4.5.8.10, 4.5.9.1,
4.5.9.2, 4.5.9.3, 4.5.9.4, 4.5.9.5, 4.5.9.6, 4.5.9.7, 4.5.9.8, 4.5.9.9, 4.5.9.10, 4.5.10.1, 4.5.10.2,
4.5.10.3, 4.5.10.4, 4.5.10.5, 4.5.10.6, 4.5.10.7, 4.5.10.8, 4.5.10.9, 4.5.10.10, 4.6.1.1, 4.6.1.2,
4.6.1.3, 4.6.1.4, 4.6.1.5, 4.6.1.6, 4.6.1.7, 4.6.1.8, 4.6.1.9, 4.6.1.10, 4.6.2.1, 4.6.2.2, 4.6.2.3,
4.6.2.4, 4.6.2.5, 4.6.2.6, 4.6.2.7, 4.6.2.8, 4.6.2.9, 4.6.2.10, 4.6.3.1, 4.6.3.2, 4.6.3.3, 4.6.3.4,
4.6.3.5, 4.6.3.6, 4.6.3.7, 4.6.3.8, 4.6.3.9, 4.6.3.10, 4.6.4.1, 4.6.4.2, 4.6.4.3, 4.6.4.4, 4.6.4.5,
4.6.4.6, 4.6.4.7, 4.6.4.8, 4.6.4.9, 4.6.4.10, 4.6.5.1, 4.6.5.2, 4.6.5.3, 4.6.5.4, 4.6.5.5, 4.6.5.6,
4.6.5.7, 4.6.5.8, 4.6.5.9, 4.6.5.10, 4.6.6.1, 4.6.6.2, 4.6.6.3, 4.6.6.4, 4.6.6.5, 4.6.6.6, 4.6.6.7,
4.6.6.8, 4.6.6.9, 4.6.6.10, 4.6.7.1, 4.6.7.2, 4.6.7.3, 4.6.7.4, 4.6.7.5, 4.6.7.6, 4.6.7.7, 4.6.7.8,
4.6.7.9, 4.6.7.10, 4.6.8.1, 4.6.8.2, 4.6.8.3, 4.6.8.4, 4.6.8.5, 4.6.8.6, 4.6.8.7, 4.6.8.8, 4.6.8.9,
4.6.8.10, 4.6.9.1, 4.6.9.2, 4.6.9.3, 4.6.9.4, 4.6.9.5, 4.6.9.6, 4.6.9.7, 4.6.9.8, 4.6.9.9, 4.6.9.10,
4.6.10.1, 4.6.10.2, 4.6.10.3, 4.6.10.4, 4.6.10.5, 4.6.10.6, 4.6.10.7, 4.6.10.8, 4.6.10.9, 4.6.10.10,
4.7.1.1, 4.7.1.2, 4.7.1.3, 4.7.1.4, 4.7.1.5, 4.7.1.6, 4.7.1.7, 4.7.1.8, 4.7.1.9, 4.7.1.10, 4.7.2.1,
4.7.2.2, 4.7.2.3, 4.7.2.4, 4.7.2.5, 4.7.2.6, 4.7.2.7, 4.7.2.8, 4.7.2.9, 4.7.2.10, 4.7.3.1, 4.7.3.2,
4.7.3.3, 4.7.3.4, 4.7.3.5, 4.7.3.6, 4.7.3.7, 4.7.3.8, 4.7.3.9, 4.7.3.10, 4.7.4.1, 4.7.4.2, 4.7.4.3,
4.7.4.4, 4.7.4.5, 4.7.4.6, 4.7.4.7, 4.7.4.8, 4.7.4.9, 4.7.4.10, 4.7.5.1, 4.7.5.2, 4.7.5.3, 4.7.5.4,
4.7.5.5, 4.7.5.6, 4.7.5.7, 4.7.5.8, 4.7.5.9, 4.7.5.10, 4.7.6.1, 4.7.6.2, 4.7.6.3, 4.7.6.4, 4.7.6.5,
4.7.6.6, 4.7.6.7, 4.7.6.8, 4.7.6.9, 4.7.6.10, 4.7.7.1, 4.7.7.2, 4.7.7.3, 4.7.7.4, 4.7.7.5, 4.7.7.6,
4.7.7.7, 4.7.7.8, 4.7.7.9, 4.7.7.10, 4.7.8.1, 4.7.8.2, 4.7.8.3, 4.7.8.4, 4.7.8.5, 4.7.8.6, 4.7.8.7,
4.7.8.8, 4.7.8.9, 4.7.8.10, 4.7.9.1, 4.7.9.2, 4.7.9.3, 4.7.9.4, 4.7.9.5, 4.7.9.6, 4.7.9.7, 4.7.9.8,
4.7.9.9, 4.7.9.10, 4.7.10.1, 4.7.10.2, 4.7.10.3, 4.7.10.4, 4.7.10.5, 4.7.10.6, 4.7.10.7, 4.7.10.8,
4.7.10.9, 4.7.10.10, 4.8.1.1, 4.8.1.2, 4.8.1.3, 4.8.1.4, 4.8.1.5, 4.8.1.6, 4.8.1.7, 4.8.1.8, 4.8.1.9,
4.8.1.10, 4.8.2.1, 4.8.2.2, 4.8.2.3, 4.8.2.4, 4.8.2.5, 4.8.2.6, 4.8.2.7, 4.8.2.8, 4.8.2.9, 4.8.2.10,
4.8.3.1, 4.8.3.2, 4.8.3.3, 4.8.3.4, 4.8.3.5, 4.8.3.6, 4.8.3.7, 4.8.3.8, 4.8.3.9, 4.8.3.10, 4.8.4.1,
4.8.4.2, 4.8.4.3, 4.8.4.4, 4.8.4.5, 4.8.4.6, 4.8.4.7, 4.8.4.8, 4.8.4.9, 4.8.4.10, 4.8.5.1, 4.8.5.2,
4.8.5.3, 4.8.5.4, 4.8.5.5, 4.8.5.6, 4.8.5.7, 4.8.5.8, 4.8.5.9, 4.8.5.10, 4.8.6.1, 4.8.6.2, 4.8.6.3,
4.8.6.4, 4.8.6.5, 4.8.6.6, 4.8.6.7, 4.8.6.8, 4.8.6.9, 4.8.6.10, 4.8.7.1, 4.8.7.2, 4.8.7.3, 4.8.7.4,
4.8.7.5, 4.8.7.6, 4.8.7.7, 4.8.7.8, 4.8.7.9, 4.8.7.10, 4.8.8.1, 4.8.8.2, 4.8.8.3, 4.8.8.4, 4.8.8.5,
4.8.8.6, 4.8.8.7, 4.8.8.8, 4.8.8.9, 4.8.8.10, 4.8.9.1, 4.8.9.2, 4.8.9.3, 4.8.9.4, 4.8.9.5, 4.8.9.6,
4.8.9.7, 4.8.9.8, 4.8.9.9, 4.8.9.10, 4.8.10.1, 4.8.10.2, 4.8.10.3, 4.8.10.4, 4.8.10.5, 4.8.10.6,
4.8.10.7, 4.8.10.8, 4.8.10.9, 4.8.10.10, 4.9.1.1, 4.9.1.2, 4.9.1.3, 4.9.1.4, 4.9.1.5, 4.9.1.6, 4.9.1.7,
4.9.1.8, 4.9.1.9, 4.9.1.10, 4.9.2.1, 4.9.2.2, 4.9.2.3, 4.9.2.4, 4.9.2.5, 4.9.2.6, 4.9.2.7, 4.9.2.8,
4.9.2.9, 4.9.2.10, 4.9.3.1, 4.9.3.2, 4.9.3.3, 4.9.3.4, 4.9.3.5, 4.9.3.6, 4.9.3.7, 4.9.3.8, 4.9.3.9,
4.9.3.10, 4.9.4.1, 4.9.4.2, 4.9.4.3, 4.9.4.4, 4.9.4.5, 4.9.4.6, 4.9.4.7, 4.9.4.8, 4.9.4.9, 4.9.4.10,
4.9.5.1, 4.9.5.2, 4.9.5.3, 4.9.5.4, 4.9.5.5, 4.9.5.6, 4.9.5.7, 4.9.5.8, 4.9.5.9, 4.9.5.10, 4.9.6.1,
4.9.6.2, 4.9.6.3, 4.9.6.4, 4.9.6.5, 4.9.6.6, 4.9.6.7, 4.9.6.8, 4.9.6.9, 4.9.6.10, 4.9.7.1, 4.9.7.2,
4.9.7.3, 4.9.7.4, 4.9.7.5, 4.9.7.6, 4.9.7.7, 4.9.7.8, 4.9.7.9, 4.9.7.10, 4.9.8.1, 4.9.8.2, 4.9.8.3,
4.9.8.4, 4.9.8.5, 4.9.8.6, 4.9.8.7, 4.9.8.8, 4.9.8.9, 4.9.8.10, 4.9.9.1, 4.9.9.2, 4.9.9.3, 4.9.9.4,
4.9.9.5, 4.9.9.6, 4.9.9.7, 4.9.9.8, 4.9.9.9, 4.9.9.10, 4.9.10.1, 4.9.10.2, 4.9.10.3, 4.9.10.4, 4.9.10.5,
4.9.10.6, 4.9.10.7, 4.9.10.8, 4.9.10.9, 4.9.10.10, 4.10.1.1, 4.10.1.2, 4.10.1.3, 4.10.1.4, 4.10.1.5,
4.10.1.6, 4.10.1.7, 4.10.1.8, 4.10.1.9, 4.10.1.10, 4.10.2.1, 4.10.2.2, 4.10.2.3, 4.10.2.4, 4.10.2.5,
4.10.2.6, 4.10.2.7, 4.10.2.8, 4.10.2.9, 4.10.2.10, 4.10.3.1, 4.10.3.2, 4.10.3.3, 4.10.3.4, 4.10.3.5,
4.10.3.6, 4.10.3.7, 4.10.3.8, 4.10.3.9, 4.10.3.10, 4.10.4.1, 4.10.4.2, 4.10.4.3, 4.10.4.4, 4.10.4.5,
4.10.4.6, 4.10.4.7, 4.10.4.8, 4.10.4.9, 4.10.4.10, 4.10.5.1, 4.10.5.2, 4.10.5.3, 4.10.5.4, 4.10.5.5,
4.10.5.6, 4.10.5.7, 4.10.5.8, 4.10.5.9, 4.10.5.10, 4.10.6.1, 4.10.6.2, 4.10.6.3, 4.10.6.4, 4.10.6.5,
4.10.6.6, 4.10.6.7, 4.10.6.8, 4.10.6.9, 4.10.6.10, 4.10.7.1, 4.10.7.2, 4.10.7.3, 4.10.7.4, 4.10.7.5,
4.10.7.6, 4.10.7.7, 4.10.7.8, 4.10.7.9, 4.10.7.10, 4.10.8.1, 4.10.8.2, 4.10.8.3, 4.10.8.4, 4.10.8.5,
4.10.8.6, 4.10.8.7, 4.10.8.8, 4.10.8.9, 4.10.8.10, 4.10.9.1, 4.10.9.2, 4.10.9.3, 4.10.9.4, 4.10.9.5,
4.10.9.6, 4.10.9.7, 4.10.9.8, 4.10.9.9, 4.10.9.10, 4.10.10.1, 4.10.10.2, 4.10.10.3, 4.10.10.4,
4.10.10.5, 4.10.10.6, 4.10.10.7, 4.10.10.8, 4.10.10.9, 4.10.10.10, 5.1.1.1, 5.1.1.2, 5.1.1.3, 5.1.1.4,
5.1.1.5, 5.1.1.6, 5.1.1.7, 5.1.1.8, 5.1.1.9, 5.1.1.10, 5.1.2.1, 5.1.2.2, 5.1.2.3, 5.1.2.4, 5.1.2.5,
5.1.2.6, 5.1.2.7, 5.1.2.8, 5.1.2.9, 5.1.2.10, 5.1.3.1, 5.1.3.2, 5.1.3.3, 5.1.3.4, 5.1.3.5, 5.1.3.6,
5.1.3.7, 5.1.3.8, 5.1.3.9, 5.1.3.10, 5.1.4.1, 5.1.4.2, 5.1.4.3, 5.1.4.4, 5.1.4.5, 5.1.4.6, 5.1.4.7,
5.1.4.8, 5.1.4.9, 5.1.4.10, 5.1.5.1, 5.1.5.2, 5.1.5.3, 5.1.5.4, 5.1.5.5, 5.1.5.6, 5.1.5.7, 5.1.5.8,
5.1.5.9, 5.1.5.10, 5.1.6.1, 5.1.6.2, 5.1.6.3, 5.1.6.4, 5.1.6.5, 5.1.6.6, 5.1.6.7, 5.1.6.8, 5.1.6.9,
5.1.6.10, 5.1.7.1, 5.1.7.2, 5.1.7.3, 5.1.7.4, 5.1.7.5, 5.1.7.6, 5.1.7.7, 5.1.7.8, 5.1.7.9, 5.1.7.10,
5.1.8.1, 5.1.8.2, 5.1.8.3, 5.1.8.4, 5.1.8.5, 5.1.8.6, 5.1.8.7, 5.1.8.8, 5.1.8.9, 5.1.8.10, 5.1.9.1,
5.1.9.2, 5.1.9.3, 5.1.9.4, 5.1.9.5, 5.1.9.6, 5.1.9.7, 5.1.9.8, 5.1.9.9, 5.1.9.10, 5.1.10.1, 5.1.10.2,
5.1.10.3, 5.1.10.4, 5.1.10.5, 5.1.10.6, 5.1.10.7, 5.1.10.8, 5.1.10.9, 5.1.10.10, 5.2.1.1, 5.2.1.2,
5.2.1.3, 5.2.1.4, 5.2.1.5, 5.2.1.6, 5.2.1.7, 5.2.1.8, 5.2.1.9, 5.2.1.10, 5.2.2.1, 5.2.2.2, 5.2.2.3,
5.2.2.4, 5.2.2.5, 5.2.2.6, 5.2.2.7, 5.2.2.8, 5.2.2.9, 5.2.2.10, 5.2.3.1, 5.2.3.2, 5.2.3.3, 5.2.3.4,
5.2.3.5, 5.2.3.6, 5.2.3.7, 5.2.3.8, 5.2.3.9, 5.2.3.10, 5.2.4.1, 5.2.4.2, 5.2.4.3, 5.2.4.4, 5.2.4.5,
5.2.4.6, 5.2.4.7, 5.2.4.8, 5.2.4.9, 5.2.4.10, 5.2.5.1, 5.2.5.2, 5.2.5.3, 5.2.5.4, 5.2.5.5, 5.2.5.6,
5.2.5.7, 5.2.5.8, 5.2.5.9, 5.2.5.10, 5.2.6.1, 5.2.6.2, 5.2.6.3, 5.2.6.4, 5.2.6.5, 5.2.6.6, 5.2.6.7,
5.2.6.8, 5.2.6.9, 5.2.6.10, 5.2.7.1, 5.2.7.2, 5.2.7.3, 5.2.7.4, 5.2.7.5, 5.2.7.6, 5.2.7.7, 5.2.7.8,
5.2.7.9, 5.2.7.10, 5.2.8.1, 5.2.8.2, 5.2.8.3, 5.2.8.4, 5.2.8.5, 5.2.8.6, 5.2.8.7, 5.2.8.8, 5.2.8.9,
5.2.8.10, 5.2.9.1, 5.2.9.2, 5.2.9.3, 5.2.9.4, 5.2.9.5, 5.2.9.6, 5.2.9.7, 5.2.9.8, 5.2.9.9, 5.2.9.10,
5.2.10.1, 5.2.10.2, 5.2.10.3, 5.2.10.4, 5.2.10.5, 5.2.10.6, 5.2.10.7, 5.2.10.8, 5.2.10.9, 5.2.10.10,
5.3.1.1, 5.3.1.2, 5.3.1.3, 5.3.1.4, 5.3.1.5, 5.3.1.6, 5.3.1.7, 5.3.1.8, 5.3.1.9, 5.3.1.10, 5.3.2.1,
5.3.2.2, 5.3.2.3, 5.3.2.4, 5.3.2.5, 5.3.2.6, 5.3.2.7, 5.3.2.8, 5.3.2.9, 5.3.2.10, 5.3.3.1, 5.3.3.2,
5.3.3.3, 5.3.3.4, 5.3.3.5, 5.3.3.6, 5.3.3.7, 5.3.3.8, 5.3.3.9, 5.3.3.10, 5.3.4.1, 5.3.4.2, 5.3.4.3,
5.3.4.4, 5.3.4.5, 5.3.4.6, 5.3.4.7, 5.3.4.8, 5.3.4.9, 5.3.4.10, 5.3.5.1, 5.3.5.2, 5.3.5.3, 5.3.5.4,
5.3.5.5, 5.3.5.6, 5.3.5.7, 5.3.5.8, 5.3.5.9, 5.3.5.10, 5.3.6.1, 5.3.6.2, 5.3.6.3, 5.3.6.4, 5.3.6.5,
5.3.6.6, 5.3.6.7, 5.3.6.8, 5.3.6.9, 5.3.6.10, 5.3.7.1, 5.3.7.2, 5.3.7.3, 5.3.7.4, 5.3.7.5, 5.3.7.6,
5.3.7.7, 5.3.7.8, 5.3.7.9, 5.3.7.10, 5.3.8.1, 5.3.8.2, 5.3.8.3, 5.3.8.4, 5.3.8.5, 5.3.8.6, 5.3.8.7,
5.3.8.8, 5.3.8.9, 5.3.8.10, 5.3.9.1, 5.3.9.2, 5.3.9.3, 5.3.9.4, 5.3.9.5, 5.3.9.6, 5.3.9.7, 5.3.9.8,
5.3.9.9, 5.3.9.10, 5.3.10.1, 5.3.10.2, 5.3.10.3, 5.3.10.4, 5.3.10.5, 5.3.10.6, 5.3.10.7, 5.3.10.8,
5.3.10.9, 5.3.10.10, 5.4.1.1, 5.4.1.2, 5.4.1.3, 5.4.1.4, 5.4.1.5, 5.4.1.6, 5.4.1.7, 5.4.1.8, 5.4.1.9,
5.4.1.10, 5.4.2.1, 5.4.2.2, 5.4.2.3, 5.4.2.4, 5.4.2.5, 5.4.2.6, 5.4.2.7, 5.4.2.8, 5.4.2.9, 5.4.2.10,
5.4.3.1, 5.4.3.2, 5.4.3.3, 5.4.3.4, 5.4.3.5, 5.4.3.6, 5.4.3.7, 5.4.3.8, 5.4.3.9, 5.4.3.10, 5.4.4.1,
5.4.4.2, 5.4.4.3, 5.4.4.4, 5.4.4.5, 5.4.4.6, 5.4.4.7, 5.4.4.8, 5.4.4.9, 5.4.4.10, 5.4.5.1, 5.4.5.2,
5.4.5.3, 5.4.5.4, 5.4.5.5, 5.4.5.6, 5.4.5.7, 5.4.5.8, 5.4.5.9, 5.4.5.10, 5.4.6.1, 5.4.6.2, 5.4.6.3,
5.4.6.4, 5.4.6.5, 5.4.6.6, 5.4.6.7, 5.4.6.8, 5.4.6.9, 5.4.6.10, 5.4.7.1, 5.4.7.2, 5.4.7.3, 5.4.7.4,
5.4.7.5, 5.4.7.6, 5.4.7.7, 5.4.7.8, 5.4.7.9, 5.4.7.10, 5.4.8.1, 5.4.8.2, 5.4.8.3, 5.4.8.4, 5.4.8.5,
5.4.8.6, 5.4.8.7, 5.4.8.8, 5.4.8.9, 5.4.8.10, 5.4.9.1, 5.4.9.2, 5.4.9.3, 5.4.9.4, 5.4.9.5, 5.4.9.6,
5.4.9.7, 5.4.9.8, 5.4.9.9, 5.4.9.10, 5.4.10.1, 5.4.10.2, 5.4.10.3, 5.4.10.4, 5.4.10.5, 5.4.10.6,
5.4.10.7, 5.4.10.8, 5.4.10.9, 5.4.10.10, 5.5.1.1, 5.5.1.2, 5.5.1.3, 5.5.1.4, 5.5.1.5, 5.5.1.6, 5.5.1.7,
5.5.1.8, 5.5.1.9, 5.5.1.10, 5.5.2.1, 5.5.2.2, 5.5.2.3, 5.5.2.4, 5.5.2.5, 5.5.2.6, 5.5.2.7, 5.5.2.8,
5.5.2.9, 5.5.2.10, 5.5.3.1, 5.5.3.2, 5.5.3.3, 5.5.3.4, 5.5.3.5, 5.5.3.6, 5.5.3.7, 5.5.3.8, 5.5.3.9,
5.5.3.10, 5.5.4.1, 5.5.4.2, 5.5.4.3, 5.5.4.4, 5.5.4.5, 5.5.4.6, 5.5.4.7, 5.5.4.8, 5.5.4.9, 5.5.4.10,
5.5.5.1, 5.5.5.2, 5.5.5.3, 5.5.5.4, 5.5.5.5, 5.5.5.6, 5.5.5.7, 5.5.5.8, 5.5.5.9, 5.5.5.10, 5.5.6.1,
5.5.6.2, 5.5.6.3, 5.5.6.4, 5.5.6.5, 5.5.6.6, 5.5.6.7, 5.5.6.8, 5.5.6.9, 5.5.6.10, 5.5.7.1, 5.5.7.2,
5.5.7.3, 5.5.7.4, 5.5.7.5, 5.5.7.6, 5.5.7.7, 5.5.7.8, 5.5.7.9, 5.5.7.10, 5.5.8.1, 5.5.8.2, 5.5.8.3,
5.5.8.4, 5.5.8.5, 5.5.8.6, 5.5.8.7, 5.5.8.8, 5.5.8.9, 5.5.8.10, 5.5.9.1, 5.5.9.2, 5.5.9.3, 5.5.9.4,
5.5.9.5, 5.5.9.6, 5.5.9.7, 5.5.9.8, 5.5.9.9, 5.5.9.10, 5.5.10.1, 5.5.10.2, 5.5.10.3, 5.5.10.4, 5.5.10.5,
5.5.10.6, 5.5.10.7, 5.5.10.8, 5.5.10.9, 5.5.10.10, 5.6.1.1, 5.6.1.2, 5.6.1.3, 5.6.1.4, 5.6.1.5, 5.6.1.6,
5.6.1.7, 5.6.1.8, 5.6.1.9, 5.6.1.10, 5.6.2.1, 5.6.2.2, 5.6.2.3, 5.6.2.4, 5.6.2.5, 5.6.2.6, 5.6.2.7,
5.6.2.8, 5.6.2.9, 5.6.2.10, 5.6.3.1, 5.6.3.2, 5.6.3.3, 5.6.3.4, 5.6.3.5, 5.6.3.6, 5.6.3.7, 5.6.3.8,
5.6.3.9, 5.6.3.10, 5.6.4.1, 5.6.4.2, 5.6.4.3, 5.6.4.4, 5.6.4.5, 5.6.4.6, 5.6.4.7, 5.6.4.8, 5.6.4.9,
5.6.4.10, 5.6.5.1, 5.6.5.2, 5.6.5.3, 5.6.5.4, 5.6.5.5, 5.6.5.6, 5.6.5.7, 5.6.5.8, 5.6.5.9, 5.6.5.10,
5.6.6.1, 5.6.6.2, 5.6.6.3, 5.6.6.4, 5.6.6.5, 5.6.6.6, 5.6.6.7, 5.6.6.8, 5.6.6.9, 5.6.6.10, 5.6.7.1,
5.6.7.2, 5.6.7.3, 5.6.7.4, 5.6.7.5, 5.6.7.6, 5.6.7.7, 5.6.7.8, 5.6.7.9, 5.6.7.10, 5.6.8.1, 5.6.8.2,
5.6.8.3, 5.6.8.4, 5.6.8.5, 5.6.8.6, 5.6.8.7, 5.6.8.8, 5.6.8.9, 5.6.8.10, 5.6.9.1, 5.6.9.2, 5.6.9.3,
5.6.9.4, 5.6.9.5, 5.6.9.6, 5.6.9.7, 5.6.9.8, 5.6.9.9, 5.6.9.10, 5.6.10.1, 5.6.10.2, 5.6.10.3, 5.6.10.4,
5.6.10.5, 5.6.10.6, 5.6.10.7, 5.6.10.8, 5.6.10.9, 5.6.10.10, 5.7.1.1, 5.7.1.2, 5.7.1.3, 5.7.1.4,
5.7.1.5, 5.7.1.6, 5.7.1.7, 5.7.1.8, 5.7.1.9, 5.7.1.10, 5.7.2.1, 5.7.2.2, 5.7.2.3, 5.7.2.4, 5.7.2.5,
5.7.2.6, 5.7.2.7, 5.7.2.8, 5.7.2.9, 5.7.2.10, 5.7.3.1, 5.7.3.2, 5.7.3.3, 5.7.3.4, 5.7.3.5, 5.7.3.6,
5.7.3.7, 5.7.3.8, 5.7.3.9, 5.7.3.10, 5.7.4.1, 5.7.4.2, 5.7.4.3, 5.7.4.4, 5.7.4.5, 5.7.4.6, 5.7.4.7,
5.7.4.8, 5.7.4.9, 5.7.4.10, 5.7.5.1, 5.7.5.2, 5.7.5.3, 5.7.5.4, 5.7.5.5, 5.7.5.6, 5.7.5.7, 5.7.5.8,
5.7.5.9, 5.7.5.10, 5.7.6.1, 5.7.6.2, 5.7.6.3, 5.7.6.4, 5.7.6.5, 5.7.6.6, 5.7.6.7, 5.7.6.8, 5.7.6.9,
5.7.6.10, 5.7.7.1, 5.7.7.2, 5.7.7.3, 5.7.7.4, 5.7.7.5, 5.7.7.6, 5.7.7.7, 5.7.7.8, 5.7.7.9, 5.7.7.10,
5.7.8.1, 5.7.8.2, 5.7.8.3, 5.7.8.4, 5.7.8.5, 5.7.8.6, 5.7.8.7, 5.7.8.8, 5.7.8.9, 5.7.8.10, 5.7.9.1,
5.7.9.2, 5.7.9.3, 5.7.9.4, 5.7.9.5, 5.7.9.6, 5.7.9.7, 5.7.9.8, 5.7.9.9, 5.7.9.10, 5.7.10.1, 5.7.10.2,
5.7.10.3, 5.7.10.4, 5.7.10.5, 5.7.10.6, 5.7.10.7, 5.7.10.8, 5.7.10.9, 5.7.10.10, 5.8.1.1, 5.8.1.2,
5.8.1.3, 5.8.1.4, 5.8.1.5, 5.8.1.6, 5.8.1.7, 5.8.1.8, 5.8.1.9, 5.8.1.10, 5.8.2.1, 5.8.2.2, 5.8.2.3,
5.8.2.4, 5.8.2.5, 5.8.2.6, 5.8.2.7, 5.8.2.8, 5.8.2.9, 5.8.2.10, 5.8.3.1, 5.8.3.2, 5.8.3.3, 5.8.3.4,
5.8.3.5, 5.8.3.6, 5.8.3.7, 5.8.3.8, 5.8.3.9, 5.8.3.10, 5.8.4.1, 5.8.4.2, 5.8.4.3, 5.8.4.4, 5.8.4.5,
5.8.4.6, 5.8.4.7, 5.8.4.8, 5.8.4.9, 5.8.4.10, 5.8.5.1, 5.8.5.2, 5.8.5.3, 5.8.5.4, 5.8.5.5, 5.8.5.6,
5.8.5.7, 5.8.5.8, 5.8.5.9, 5.8.5.10, 5.8.6.1, 5.8.6.2, 5.8.6.3, 5.8.6.4, 5.8.6.5, 5.8.6.6, 5.8.6.7,
5.8.6.8, 5.8.6.9, 5.8.6.10, 5.8.7.1, 5.8.7.2, 5.8.7.3, 5.8.7.4, 5.8.7.5, 5.8.7.6, 5.8.7.7, 5.8.7.8,
5.8.7.9, 5.8.7.10, 5.8.8.1, 5.8.8.2, 5.8.8.3, 5.8.8.4, 5.8.8.5, 5.8.8.6, 5.8.8.7, 5.8.8.8, 5.8.8.9,
5.8.8.10, 5.8.9.1, 5.8.9.2, 5.8.9.3, 5.8.9.4, 5.8.9.5, 5.8.9.6, 5.8.9.7, 5.8.9.8, 5.8.9.9, 5.8.9.10,
5.8.10.1, 5.8.10.2, 5.8.10.3, 5.8.10.4, 5.8.10.5, 5.8.10.6, 5.8.10.7, 5.8.10.8, 5.8.10.9, 5.8.10.10,
5.9.1.1, 5.9.1.2, 5.9.1.3, 5.9.1.4, 5.9.1.5, 5.9.1.6, 5.9.1.7, 5.9.1.8, 5.9.1.9, 5.9.1.10, 5.9.2.1,
5.9.2.2, 5.9.2.3, 5.9.2.4, 5.9.2.5, 5.9.2.6, 5.9.2.7, 5.9.2.8, 5.9.2.9, 5.9.2.10, 5.9.3.1, 5.9.3.2,
5.9.3.3, 5.9.3.4, 5.9.3.5, 5.9.3.6, 5.9.3.7, 5.9.3.8, 5.9.3.9, 5.9.3.10, 5.9.4.1, 5.9.4.2, 5.9.4.3,
5.9.4.4, 5.9.4.5, 5.9.4.6, 5.9.4.7, 5.9.4.8, 5.9.4.9, 5.9.4.10, 5.9.5.1, 5.9.5.2, 5.9.5.3, 5.9.5.4,
5.9.5.5, 5.9.5.6, 5.9.5.7, 5.9.5.8, 5.9.5.9, 5.9.5.10, 5.9.6.1, 5.9.6.2, 5.9.6.3, 5.9.6.4, 5.9.6.5,
5.9.6.6, 5.9.6.7, 5.9.6.8, 5.9.6.9, 5.9.6.10, 5.9.7.1, 5.9.7.2, 5.9.7.3, 5.9.7.4, 5.9.7.5, 5.9.7.6,
5.9.7.7, 5.9.7.8, 5.9.7.9, 5.9.7.10, 5.9.8.1, 5.9.8.2, 5.9.8.3, 5.9.8.4, 5.9.8.5, 5.9.8.6, 5.9.8.7,
5.9.8.8, 5.9.8.9, 5.9.8.10, 5.9.9.1, 5.9.9.2, 5.9.9.3, 5.9.9.4, 5.9.9.5, 5.9.9.6, 5.9.9.7, 5.9.9.8,
5.9.9.9, 5.9.9.10, 5.9.10.1, 5.9.10.2, 5.9.10.3, 5.9.10.4, 5.9.10.5, 5.9.10.6, 5.9.10.7, 5.9.10.8,
5.9.10.9, 5.9.10.10, 5.10.1.1, 5.10.1.2, 5.10.1.3, 5.10.1.4, 5.10.1.5, 5.10.1.6, 5.10.1.7, 5.10.1.8,
5.10.1.9, 5.10.1.10, 5.10.2.1, 5.10.2.2, 5.10.2.3, 5.10.2.4, 5.10.2.5, 5.10.2.6, 5.10.2.7, 5.10.2.8,
5.10.2.9, 5.10.2.10, 5.10.3.1, 5.10.3.2, 5.10.3.3, 5.10.3.4, 5.10.3.5, 5.10.3.6, 5.10.3.7, 5.10.3.8,
5.10.3.9, 5.10.3.10, 5.10.4.1, 5.10.4.2, 5.10.4.3, 5.10.4.4, 5.10.4.5, 5.10.4.6, 5.10.4.7, 5.10.4.8,
5.10.4.9, 5.10.4.10, 5.10.5.1, 5.10.5.2, 5.10.5.3, 5.10.5.4, 5.10.5.5, 5.10.5.6, 5.10.5.7, 5.10.5.8,
5.10.5.9, 5.10.5.10, 5.10.6.1, 5.10.6.2, 5.10.6.3, 5.10.6.4, 5.10.6.5, 5.10.6.6, 5.10.6.7, 5.10.6.8,
5.10.6.9, 5.10.6.10, 5.10.7.1, 5.10.7.2, 5.10.7.3, 5.10.7.4, 5.10.7.5, 5.10.7.6, 5.10.7.7, 5.10.7.8,
5.10.7.9, 5.10.7.10, 5.10.8.1, 5.10.8.2, 5.10.8.3, 5.10.8.4, 5.10.8.5, 5.10.8.6, 5.10.8.7, 5.10.8.8,
5.10.8.9, 5.10.8.10, 5.10.9.1, 5.10.9.2, 5.10.9.3, 5.10.9.4, 5.10.9.5, 5.10.9.6, 5.10.9.7, 5.10.9.8,
5.10.9.9, 5.10.9.10, 5.10.10.1, 5.10.10.2, 5.10.10.3, 5.10.10.4, 5.10.10.5, 5.10.10.6, 5.10.10.7,
5.10.10.8, 5.10.10.9, 5.10.10.10, 6.1.1.1, 6.1.1.2, 6.1.1.3, 6.1.1.4, 6.1.1.5, 6.1.1.6, 6.1.1.7,
6.1.1.8, 6.1.1.9, 6.1.1.10, 6.1.2.1, 6.1.2.2, 6.1.2.3, 6.1.2.4, 6.1.2.5, 6.1.2.6, 6.1.2.7, 6.1.2.8,
6.1.2.9, 6.1.2.10, 6.1.3.1, 6.1.3.2, 6.1.3.3, 6.1.3.4, 6.1.3.5, 6.1.3.6, 6.1.3.7, 6.1.3.8, 6.1.3.9,
6.1.3.10, 6.1.4.1, 6.1.4.2, 6.1.4.3, 6.1.4.4, 6.1.4.5, 6.1.4.6, 6.1.4.7, 6.1.4.8, 6.1.4.9, 6.1.4.10,
6.1.5.1, 6.1.5.2, 6.1.5.3, 6.1.5.4, 6.1.5.5, 6.1.5.6, 6.1.5.7, 6.1.5.8, 6.1.5.9, 6.1.5.10, 6.1.6.1,
6.1.6.2, 6.1.6.3, 6.1.6.4, 6.1.6.5, 6.1.6.6, 6.1.6.7, 6.1.6.8, 6.1.6.9, 6.1.6.10, 6.1.7.1, 6.1.7.2,
6.1.7.3, 6.1.7.4, 6.1.7.5, 6.1.7.6, 6.1.7.7, 6.1.7.8, 6.1.7.9, 6.1.7.10, 6.1.8.1, 6.1.8.2, 6.1.8.3,
6.1.8.4, 6.1.8.5, 6.1.8.6, 6.1.8.7, 6.1.8.8, 6.1.8.9, 6.1.8.10, 6.1.9.1, 6.1.9.2, 6.1.9.3, 6.1.9.4,
6.1.9.5, 6.1.9.6, 6.1.9.7, 6.1.9.8, 6.1.9.9, 6.1.9.10, 6.1.10.1, 6.1.10.2, 6.1.10.3, 6.1.10.4, 6.1.10.5,
6.1.10.6, 6.1.10.7, 6.1.10.8, 6.1.10.9, 6.1.10.10, 6.2.1.1, 6.2.1.2, 6.2.1.3, 6.2.1.4, 6.2.1.5, 6.2.1.6,
6.2.1.7, 6.2.1.8, 6.2.1.9, 6.2.1.10, 6.2.2.1, 6.2.2.2, 6.2.2.3, 6.2.2.4, 6.2.2.5, 6.2.2.6, 6.2.2.7,
6.2.2.8, 6.2.2.9, 6.2.2.10, 6.2.3.1, 6.2.3.2, 6.2.3.3, 6.2.3.4, 6.2.3.5, 6.2.3.6, 6.2.3.7, 6.2.3.8,
6.2.3.9, 6.2.3.10, 6.2.4.1, 6.2.4.2, 6.2.4.3, 6.2.4.4, 6.2.4.5, 6.2.4.6, 6.2.4.7, 6.2.4.8, 6.2.4.9,
6.2.4.10, 6.2.5.1, 6.2.5.2, 6.2.5.3, 6.2.5.4, 6.2.5.5, 6.2.5.6, 6.2.5.7, 6.2.5.8, 6.2.5.9, 6.2.5.10,
6.2.6.1, 6.2.6.2, 6.2.6.3, 6.2.6.4, 6.2.6.5, 6.2.6.6, 6.2.6.7, 6.2.6.8, 6.2.6.9, 6.2.6.10, 6.2.7.1,
6.2.7.2, 6.2.7.3, 6.2.7.4, 6.2.7.5, 6.2.7.6, 6.2.7.7, 6.2.7.8, 6.2.7.9, 6.2.7.10, 6.2.8.1, 6.2.8.2,
6.2.8.3, 6.2.8.4, 6.2.8.5, 6.2.8.6, 6.2.8.7, 6.2.8.8, 6.2.8.9, 6.2.8.10, 6.2.9.1, 6.2.9.2, 6.2.9.3,
6.2.9.4, 6.2.9.5, 6.2.9.6, 6.2.9.7, 6.2.9.8, 6.2.9.9, 6.2.9.10, 6.2.10.1, 6.2.10.2, 6.2.10.3, 6.2.10.4,
6.2.10.5, 6.2.10.6, 6.2.10.7, 6.2.10.8, 6.2.10.9, 6.2.10.10, 6.3.1.1, 6.3.1.2, 6.3.1.3, 6.3.1.4,
6.3.1.5, 6.3.1.6, 6.3.1.7, 6.3.1.8, 6.3.1.9, 6.3.1.10, 6.3.2.1, 6.3.2.2, 6.3.2.3, 6.3.2.4, 6.3.2.5,
6.3.2.6, 6.3.2.7, 6.3.2.8, 6.3.2.9, 6.3.2.10, 6.3.3.1, 6.3.3.2, 6.3.3.3, 6.3.3.4, 6.3.3.5, 6.3.3.6,
6.3.3.7, 6.3.3.8, 6.3.3.9, 6.3.3.10, 6.3.4.1, 6.3.4.2, 6.3.4.3, 6.3.4.4, 6.3.4.5, 6.3.4.6, 6.3.4.7,
6.3.4.8, 6.3.4.9, 6.3.4.10, 6.3.5.1, 6.3.5.2, 6.3.5.3, 6.3.5.4, 6.3.5.5, 6.3.5.6, 6.3.5.7, 6.3.5.8,
6.3.5.9, 6.3.5.10, 6.3.6.1, 6.3.6.2, 6.3.6.3, 6.3.6.4, 6.3.6.5, 6.3.6.6, 6.3.6.7, 6.3.6.8, 6.3.6.9,
6.3.6.10, 6.3.7.1, 6.3.7.2, 6.3.7.3, 6.3.7.4, 6.3.7.5, 6.3.7.6, 6.3.7.7, 6.3.7.8, 6.3.7.9, 6.3.7.10,
6.3.8.1, 6.3.8.2, 6.3.8.3, 6.3.8.4, 6.3.8.5, 6.3.8.6, 6.3.8.7, 6.3.8.8, 6.3.8.9, 6.3.8.10, 6.3.9.1,
6.3.9.2, 6.3.9.3, 6.3.9.4, 6.3.9.5, 6.3.9.6, 6.3.9.7, 6.3.9.8, 6.3.9.9, 6.3.9.10, 6.3.10.1, 6.3.10.2,
6.3.10.3, 6.3.10.4, 6.3.10.5, 6.3.10.6, 6.3.10.7, 6.3.10.8, 6.3.10.9, 6.3.10.10, 6.4.1.1, 6.4.1.2,
6.4.1.3, 6.4.1.4, 6.4.1.5, 6.4.1.6, 6.4.1.7, 6.4.1.8, 6.4.1.9, 6.4.1.10, 6.4.2.1, 6.4.2.2, 6.4.2.3,
6.4.2.4, 6.4.2.5, 6.4.2.6, 6.4.2.7, 6.4.2.8, 6.4.2.9, 6.4.2.10, 6.4.3.1, 6.4.3.2, 6.4.3.3, 6.4.3.4,
6.4.3.5, 6.4.3.6, 6.4.3.7, 6.4.3.8, 6.4.3.9, 6.4.3.10, 6.4.4.1, 6.4.4.2, 6.4.4.3, 6.4.4.4, 6.4.4.5,
6.4.4.6, 6.4.4.7, 6.4.4.8, 6.4.4.9, 6.4.4.10, 6.4.5.1, 6.4.5.2, 6.4.5.3, 6.4.5.4, 6.4.5.5, 6.4.5.6,
6.4.5.7, 6.4.5.8, 6.4.5.9, 6.4.5.10, 6.4.6.1, 6.4.6.2, 6.4.6.3, 6.4.6.4, 6.4.6.5, 6.4.6.6, 6.4.6.7,
6.4.6.8, 6.4.6.9, 6.4.6.10, 6.4.7.1, 6.4.7.2, 6.4.7.3, 6.4.7.4, 6.4.7.5, 6.4.7.6, 6.4.7.7, 6.4.7.8,
6.4.7.9, 6.4.7.10, 6.4.8.1, 6.4.8.2, 6.4.8.3, 6.4.8.4, 6.4.8.5, 6.4.8.6, 6.4.8.7, 6.4.8.8, 6.4.8.9,
6.4.8.10, 6.4.9.1, 6.4.9.2, 6.4.9.3, 6.4.9.4, 6.4.9.5, 6.4.9.6, 6.4.9.7, 6.4.9.8, 6.4.9.9, 6.4.9.10,
6.4.10.1, 6.4.10.2, 6.4.10.3, 6.4.10.4, 6.4.10.5, 6.4.10.6, 6.4.10.7, 6.4.10.8, 6.4.10.9, 6.4.10.10,
6.5.1.1, 6.5.1.2, 6.5.1.3, 6.5.1.4, 6.5.1.5, 6.5.1.6, 6.5.1.7, 6.5.1.8, 6.5.1.9, 6.5.1.10, 6.5.2.1,
6.5.2.2, 6.5.2.3, 6.5.2.4, 6.5.2.5, 6.5.2.6, 6.5.2.7, 6.5.2.8, 6.5.2.9, 6.5.2.10, 6.5.3.1, 6.5.3.2,
6.5.3.3, 6.5.3.4, 6.5.3.5, 6.5.3.6, 6.5.3.7, 6.5.3.8, 6.5.3.9, 6.5.3.10, 6.5.4.1, 6.5.4.2, 6.5.4.3,
6.5.4.4, 6.5.4.5, 6.5.4.6, 6.5.4.7, 6.5.4.8, 6.5.4.9, 6.5.4.10, 6.5.5.1, 6.5.5.2, 6.5.5.3, 6.5.5.4,
6.5.5.5, 6.5.5.6, 6.5.5.7, 6.5.5.8, 6.5.5.9, 6.5.5.10, 6.5.6.1, 6.5.6.2, 6.5.6.3, 6.5.6.4, 6.5.6.5,
6.5.6.6, 6.5.6.7, 6.5.6.8, 6.5.6.9, 6.5.6.10, 6.5.7.1, 6.5.7.2, 6.5.7.3, 6.5.7.4, 6.5.7.5, 6.5.7.6,
6.5.7.7, 6.5.7.8, 6.5.7.9, 6.5.7.10, 6.5.8.1, 6.5.8.2, 6.5.8.3, 6.5.8.4, 6.5.8.5, 6.5.8.6, 6.5.8.7,
6.5.8.8, 6.5.8.9, 6.5.8.10, 6.5.9.1, 6.5.9.2, 6.5.9.3, 6.5.9.4, 6.5.9.5, 6.5.9.6, 6.5.9.7, 6.5.9.8,
6.5.9.9, 6.5.9.10, 6.5.10.1, 6.5.10.2, 6.5.10.3, 6.5.10.4, 6.5.10.5, 6.5.10.6, 6.5.10.7, 6.5.10.8,
6.5.10.9, 6.5.10.10, 6.6.1.1, 6.6.1.2, 6.6.1.3, 6.6.1.4, 6.6.1.5, 6.6.1.6, 6.6.1.7, 6.6.1.8, 6.6.1.9,
6.6.1.10, 6.6.2.1, 6.6.2.2, 6.6.2.3, 6.6.2.4, 6.6.2.5, 6.6.2.6, 6.6.2.7, 6.6.2.8, 6.6.2.9, 6.6.2.10,
6.6.3.1, 6.6.3.2, 6.6.3.3, 6.6.3.4, 6.6.3.5, 6.6.3.6, 6.6.3.7, 6.6.3.8, 6.6.3.9, 6.6.3.10, 6.6.4.1,
6.6.4.2, 6.6.4.3, 6.6.4.4, 6.6.4.5, 6.6.4.6, 6.6.4.7, 6.6.4.8, 6.6.4.9, 6.6.4.10, 6.6.5.1, 6.6.5.2,
6.6.5.3, 6.6.5.4, 6.6.5.5, 6.6.5.6, 6.6.5.7, 6.6.5.8, 6.6.5.9, 6.6.5.10, 6.6.6.1, 6.6.6.2, 6.6.6.3,
6.6.6.4, 6.6.6.5, 6.6.6.6, 6.6.6.7, 6.6.6.8, 6.6.6.9, 6.6.6.10, 6.6.7.1, 6.6.7.2, 6.6.7.3, 6.6.7.4,
6.6.7.5, 6.6.7.6, 6.6.7.7, 6.6.7.8, 6.6.7.9, 6.6.7.10, 6.6.8.1, 6.6.8.2, 6.6.8.3, 6.6.8.4, 6.6.8.5,
6.6.8.6, 6.6.8.7, 6.6.8.8, 6.6.8.9, 6.6.8.10, 6.6.9.1, 6.6.9.2, 6.6.9.3, 6.6.9.4, 6.6.9.5, 6.6.9.6,
6.6.9.7, 6.6.9.8, 6.6.9.9, 6.6.9.10, 6.6.10.1, 6.6.10.2, 6.6.10.3, 6.6.10.4, 6.6.10.5, 6.6.10.6,
6.6.10.7, 6.6.10.8, 6.6.10.9, 6.6.10.10, 6.7.1.1, 6.7.1.2, 6.7.1.3, 6.7.1.4, 6.7.1.5, 6.7.1.6, 6.7.1.7,
6.7.1.8, 6.7.1.9, 6.7.1.10, 6.7.2.1, 6.7.2.2, 6.7.2.3, 6.7.2.4, 6.7.2.5, 6.7.2.6, 6.7.2.7, 6.7.2.8,
6.7.2.9, 6.7.2.10, 6.7.3.1, 6.7.3.2, 6.7.3.3, 6.7.3.4, 6.7.3.5, 6.7.3.6, 6.7.3.7, 6.7.3.8, 6.7.3.9,
6.7.3.10, 6.7.4.1, 6.7.4.2, 6.7.4.3, 6.7.4.4, 6.7.4.5, 6.7.4.6, 6.7.4.7, 6.7.4.8, 6.7.4.9, 6.7.4.10,
6.7.5.1, 6.7.5.2, 6.7.5.3, 6.7.5.4, 6.7.5.5, 6.7.5.6, 6.7.5.7, 6.7.5.8, 6.7.5.9, 6.7.5.10, 6.7.6.1,
6.7.6.2, 6.7.6.3, 6.7.6.4, 6.7.6.5, 6.7.6.6, 6.7.6.7, 6.7.6.8, 6.7.6.9, 6.7.6.10, 6.7.7.1, 6.7.7.2,
6.7.7.3, 6.7.7.4, 6.7.7.5, 6.7.7.6, 6.7.7.7, 6.7.7.8, 6.7.7.9, 6.7.7.10, 6.7.8.1, 6.7.8.2, 6.7.8.3,
6.7.8.4, 6.7.8.5, 6.7.8.6, 6.7.8.7, 6.7.8.8, 6.7.8.9, 6.7.8.10, 6.7.9.1, 6.7.9.2, 6.7.9.3, 6.7.9.4,
6.7.9.5, 6.7.9.6, 6.7.9.7, 6.7.9.8, 6.7.9.9, 6.7.9.10, 6.7.10.1, 6.7.10.2, 6.7.10.3, 6.7.10.4, 6.7.10.5,
6.7.10.6, 6.7.10.7, 6.7.10.8, 6.7.10.9, 6.7.10.10, 6.8.1.1, 6.8.1.2, 6.8.1.3, 6.8.1.4, 6.8.1.5, 6.8.1.6,
6.8.1.7, 6.8.1.8, 6.8.1.9, 6.8.1.10, 6.8.2.1, 6.8.2.2, 6.8.2.3, 6.8.2.4, 6.8.2.5, 6.8.2.6, 6.8.2.7,
6.8.2.8, 6.8.2.9, 6.8.2.10, 6.8.3.1, 6.8.3.2, 6.8.3.3, 6.8.3.4, 6.8.3.5, 6.8.3.6, 6.8.3.7, 6.8.3.8,
6.8.3.9, 6.8.3.10, 6.8.4.1, 6.8.4.2, 6.8.4.3, 6.8.4.4, 6.8.4.5, 6.8.4.6, 6.8.4.7, 6.8.4.8, 6.8.4.9,
6.8.4.10, 6.8.5.1, 6.8.5.2, 6.8.5.3, 6.8.5.4, 6.8.5.5, 6.8.5.6, 6.8.5.7, 6.8.5.8, 6.8.5.9, 6.8.5.10,
6.8.6.1, 6.8.6.2, 6.8.6.3, 6.8.6.4, 6.8.6.5, 6.8.6.6, 6.8.6.7, 6.8.6.8, 6.8.6.9, 6.8.6.10, 6.8.7.1,
6.8.7.2, 6.8.7.3, 6.8.7.4, 6.8.7.5, 6.8.7.6, 6.8.7.7, 6.8.7.8, 6.8.7.9, 6.8.7.10, 6.8.8.1, 6.8.8.2,
6.8.8.3, 6.8.8.4, 6.8.8.5, 6.8.8.6, 6.8.8.7, 6.8.8.8, 6.8.8.9, 6.8.8.10, 6.8.9.1, 6.8.9.2, 6.8.9.3,
6.8.9.4, 6.8.9.5, 6.8.9.6, 6.8.9.7, 6.8.9.8, 6.8.9.9, 6.8.9.10, 6.8.10.1, 6.8.10.2, 6.8.10.3, 6.8.10.4,
6.8.10.5, 6.8.10.6, 6.8.10.7, 6.8.10.8, 6.8.10.9, 6.8.10.10, 6.9.1.1, 6.9.1.2, 6.9.1.3, 6.9.1.4,
6.9.1.5, 6.9.1.6, 6.9.1.7, 6.9.1.8, 6.9.1.9, 6.9.1.10, 6.9.2.1, 6.9.2.2, 6.9.2.3, 6.9.2.4, 6.9.2.5,
6.9.2.6, 6.9.2.7, 6.9.2.8, 6.9.2.9, 6.9.2.10, 6.9.3.1, 6.9.3.2, 6.9.3.3, 6.9.3.4, 6.9.3.5, 6.9.3.6,
6.9.3.7, 6.9.3.8, 6.9.3.9, 6.9.3.10, 6.9.4.1, 6.9.4.2, 6.9.4.3, 6.9.4.4, 6.9.4.5, 6.9.4.6, 6.9.4.7,
6.9.4.8, 6.9.4.9, 6.9.4.10, 6.9.5.1, 6.9.5.2, 6.9.5.3, 6.9.5.4, 6.9.5.5, 6.9.5.6, 6.9.5.7, 6.9.5.8,
6.9.5.9, 6.9.5.10, 6.9.6.1, 6.9.6.2, 6.9.6.3, 6.9.6.4, 6.9.6.5, 6.9.6.6, 6.9.6.7, 6.9.6.8, 6.9.6.9,
6.9.6.10, 6.9.7.1, 6.9.7.2, 6.9.7.3, 6.9.7.4, 6.9.7.5, 6.9.7.6, 6.9.7.7, 6.9.7.8, 6.9.7.9, 6.9.7.10,
6.9.8.1, 6.9.8.2, 6.9.8.3, 6.9.8.4, 6.9.8.5, 6.9.8.6, 6.9.8.7, 6.9.8.8, 6.9.8.9, 6.9.8.10, 6.9.9.1,
6.9.9.2, 6.9.9.3, 6.9.9.4, 6.9.9.5, 6.9.9.6, 6.9.9.7, 6.9.9.8, 6.9.9.9, 6.9.9.10, 6.9.10.1, 6.9.10.2,
6.9.10.3, 6.9.10.4, 6.9.10.5, 6.9.10.6, 6.9.10.7, 6.9.10.8, 6.9.10.9, 6.9.10.10, 6.10.1.1, 6.10.1.2,
6.10.1.3, 6.10.1.4, 6.10.1.5, 6.10.1.6, 6.10.1.7, 6.10.1.8, 6.10.1.9, 6.10.1.10, 6.10.2.1, 6.10.2.2,
6.10.2.3, 6.10.2.4, 6.10.2.5, 6.10.2.6, 6.10.2.7, 6.10.2.8, 6.10.2.9, 6.10.2.10, 6.10.3.1, 6.10.3.2,
6.10.3.3, 6.10.3.4, 6.10.3.5, 6.10.3.6, 6.10.3.7, 6.10.3.8, 6.10.3.9, 6.10.3.10, 6.10.4.1, 6.10.4.2,
6.10.4.3, 6.10.4.4, 6.10.4.5, 6.10.4.6, 6.10.4.7, 6.10.4.8, 6.10.4.9, 6.10.4.10, 6.10.5.1, 6.10.5.2,
6.10.5.3, 6.10.5.4, 6.10.5.5, 6.10.5.6, 6.10.5.7, 6.10.5.8, 6.10.5.9, 6.10.5.10, 6.10.6.1, 6.10.6.2,
6.10.6.3, 6.10.6.4, 6.10.6.5, 6.10.6.6, 6.10.6.7, 6.10.6.8, 6.10.6.9, 6.10.6.10, 6.10.7.1, 6.10.7.2,
6.10.7.3, 6.10.7.4, 6.10.7.5, 6.10.7.6, 6.10.7.7, 6.10.7.8, 6.10.7.9, 6.10.7.10, 6.10.8.1, 6.10.8.2,
6.10.8.3, 6.10.8.4, 6.10.8.5, 6.10.8.6, 6.10.8.7, 6.10.8.8, 6.10.8.9, 6.10.8.10, 6.10.9.1, 6.10.9.2,
6.10.9.3, 6.10.9.4, 6.10.9.5, 6.10.9.6, 6.10.9.7, 6.10.9.8, 6.10.9.9, 6.10.9.10, 6.10.10.1, 6.10.10.2,
6.10.10.3, 6.10.10.4, 6.10.10.5, 6.10.10.6, 6.10.10.7, 6.10.10.8, 6.10.10.9, 6.10.10.10, 7.1.1.1,
7.1.1.2, 7.1.1.3, 7.1.1.4, 7.1.1.5, 7.1.1.6, 7.1.1.7, 7.1.1.8, 7.1.1.9, 7.1.1.10, 7.1.2.1, 7.1.2.2,
7.1.2.3, 7.1.2.4, 7.1.2.5, 7.1.2.6, 7.1.2.7, 7.1.2.8, 7.1.2.9, 7.1.2.10, 7.1.3.1, 7.1.3.2, 7.1.3.3,
7.1.3.4, 7.1.3.5, 7.1.3.6, 7.1.3.7, 7.1.3.8, 7.1.3.9, 7.1.3.10, 7.1.4.1, 7.1.4.2, 7.1.4.3, 7.1.4.4,
7.1.4.5, 7.1.4.6, 7.1.4.7, 7.1.4.8, 7.1.4.9, 7.1.4.10, 7.1.5.1, 7.1.5.2, 7.1.5.3, 7.1.5.4, 7.1.5.5,
7.1.5.6, 7.1.5.7, 7.1.5.8, 7.1.5.9, 7.1.5.10, 7.1.6.1, 7.1.6.2, 7.1.6.3, 7.1.6.4, 7.1.6.5, 7.1.6.6,
7.1.6.7, 7.1.6.8, 7.1.6.9, 7.1.6.10, 7.1.7.1, 7.1.7.2, 7.1.7.3, 7.1.7.4, 7.1.7.5, 7.1.7.6, 7.1.7.7,
7.1.7.8, 7.1.7.9, 7.1.7.10, 7.1.8.1, 7.1.8.2, 7.1.8.3, 7.1.8.4, 7.1.8.5, 7.1.8.6, 7.1.8.7, 7.1.8.8,
7.1.8.9, 7.1.8.10, 7.1.9.1, 7.1.9.2, 7.1.9.3, 7.1.9.4, 7.1.9.5, 7.1.9.6, 7.1.9.7, 7.1.9.8, 7.1.9.9,
7.1.9.10, 7.1.10.1, 7.1.10.2, 7.1.10.3, 7.1.10.4, 7.1.10.5, 7.1.10.6, 7.1.10.7, 7.1.10.8, 7.1.10.9,
7.1.10.10, 7.2.1.1, 7.2.1.2, 7.2.1.3, 7.2.1.4, 7.2.1.5, 7.2.1.6, 7.2.1.7, 7.2.1.8, 7.2.1.9, 7.2.1.10,
7.2.2.1, 7.2.2.2, 7.2.2.3, 7.2.2.4, 7.2.2.5, 7.2.2.6, 7.2.2.7, 7.2.2.8, 7.2.2.9, 7.2.2.10, 7.2.3.1,
7.2.3.2, 7.2.3.3, 7.2.3.4, 7.2.3.5, 7.2.3.6, 7.2.3.7, 7.2.3.8, 7.2.3.9, 7.2.3.10, 7.2.4.1, 7.2.4.2,
7.2.4.3, 7.2.4.4, 7.2.4.5, 7.2.4.6, 7.2.4.7, 7.2.4.8, 7.2.4.9, 7.2.4.10, 7.2.5.1, 7.2.5.2, 7.2.5.3,
7.2.5.4, 7.2.5.5, 7.2.5.6, 7.2.5.7, 7.2.5.8, 7.2.5.9, 7.2.5.10, 7.2.6.1, 7.2.6.2, 7.2.6.3, 7.2.6.4,
7.2.6.5, 7.2.6.6, 7.2.6.7, 7.2.6.8, 7.2.6.9, 7.2.6.10, 7.2.7.1, 7.2.7.2, 7.2.7.3, 7.2.7.4, 7.2.7.5,
7.2.7.6, 7.2.7.7, 7.2.7.8, 7.2.7.9, 7.2.7.10, 7.2.8.1, 7.2.8.2, 7.2.8.3, 7.2.8.4, 7.2.8.5, 7.2.8.6,
7.2.8.7, 7.2.8.8, 7.2.8.9, 7.2.8.10, 7.2.9.1, 7.2.9.2, 7.2.9.3, 7.2.9.4, 7.2.9.5, 7.2.9.6, 7.2.9.7,
7.2.9.8, 7.2.9.9, 7.2.9.10, 7.2.10.1, 7.2.10.2, 7.2.10.3, 7.2.10.4, 7.2.10.5, 7.2.10.6, 7.2.10.7,
7.2.10.8, 7.2.10.9, 7.2.10.10, 7.3.1.1, 7.3.1.2, 7.3.1.3, 7.3.1.4, 7.3.1.5, 7.3.1.6, 7.3.1.7, 7.3.1.8,
7.3.1.9, 7.3.1.10, 7.3.2.1, 7.3.2.2, 7.3.2.3, 7.3.2.4, 7.3.2.5, 7.3.2.6, 7.3.2.7, 7.3.2.8, 7.3.2.9,
7.3.2.10, 7.3.3.1, 7.3.3.2, 7.3.3.3, 7.3.3.4, 7.3.3.5, 7.3.3.6, 7.3.3.7, 7.3.3.8, 7.3.3.9, 7.3.3.10,
7.3.4.1, 7.3.4.2, 7.3.4.3, 7.3.4.4, 7.3.4.5, 7.3.4.6, 7.3.4.7, 7.3.4.8, 7.3.4.9, 7.3.4.10, 7.3.5.1,
7.3.5.2, 7.3.5.3, 7.3.5.4, 7.3.5.5, 7.3.5.6, 7.3.5.7, 7.3.5.8, 7.3.5.9, 7.3.5.10, 7.3.6.1, 7.3.6.2,
7.3.6.3, 7.3.6.4, 7.3.6.5, 7.3.6.6, 7.3.6.7, 7.3.6.8, 7.3.6.9, 7.3.6.10, 7.3.7.1, 7.3.7.2, 7.3.7.3,
7.3.7.4, 7.3.7.5, 7.3.7.6, 7.3.7.7, 7.3.7.8, 7.3.7.9, 7.3.7.10, 7.3.8.1, 7.3.8.2, 7.3.8.3, 7.3.8.4,
7.3.8.5, 7.3.8.6, 7.3.8.7, 7.3.8.8, 7.3.8.9, 7.3.8.10, 7.3.9.1, 7.3.9.2, 7.3.9.3, 7.3.9.4, 7.3.9.5,
7.3.9.6, 7.3.9.7, 7.3.9.8, 7.3.9.9, 7.3.9.10, 7.3.10.1, 7.3.10.2, 7.3.10.3, 7.3.10.4, 7.3.10.5,
7.3.10.6, 7.3.10.7, 7.3.10.8, 7.3.10.9, 7.3.10.10, 7.4.1.1, 7.4.1.2, 7.4.1.3, 7.4.1.4, 7.4.1.5, 7.4.1.6,
7.4.1.7, 7.4.1.8, 7.4.1.9, 7.4.1.10, 7.4.2.1, 7.4.2.2, 7.4.2.3, 7.4.2.4, 7.4.2.5, 7.4.2.6, 7.4.2.7,
7.4.2.8, 7.4.2.9, 7.4.2.10, 7.4.3.1, 7.4.3.2, 7.4.3.3, 7.4.3.4, 7.4.3.5, 7.4.3.6, 7.4.3.7, 7.4.3.8,
7.4.3.9, 7.4.3.10, 7.4.4.1, 7.4.4.2, 7.4.4.3, 7.4.4.4, 7.4.4.5, 7.4.4.6, 7.4.4.7, 7.4.4.8, 7.4.4.9,
7.4.4.10, 7.4.5.1, 7.4.5.2, 7.4.5.3, 7.4.5.4, 7.4.5.5, 7.4.5.6, 7.4.5.7, 7.4.5.8, 7.4.5.9, 7.4.5.10,
7.4.6.1, 7.4.6.2, 7.4.6.3, 7.4.6.4, 7.4.6.5, 7.4.6.6, 7.4.6.7, 7.4.6.8, 7.4.6.9, 7.4.6.10, 7.4.7.1,
7.4.7.2, 7.4.7.3, 7.4.7.4, 7.4.7.5, 7.4.7.6, 7.4.7.7, 7.4.7.8, 7.4.7.9, 7.4.7.10, 7.4.8.1, 7.4.8.2,
7.4.8.3, 7.4.8.4, 7.4.8.5, 7.4.8.6, 7.4.8.7, 7.4.8.8, 7.4.8.9, 7.4.8.10, 7.4.9.1, 7.4.9.2, 7.4.9.3,
7.4.9.4, 7.4.9.5, 7.4.9.6, 7.4.9.7, 7.4.9.8, 7.4.9.9, 7.4.9.10, 7.4.10.1, 7.4.10.2, 7.4.10.3, 7.4.10.4,
7.4.10.5, 7.4.10.6, 7.4.10.7, 7.4.10.8, 7.4.10.9, 7.4.10.10, 7.5.1.1, 7.5.1.2, 7.5.1.3, 7.5.1.4,
7.5.1.5, 7.5.1.6, 7.5.1.7, 7.5.1.8, 7.5.1.9, 7.5.1.10, 7.5.2.1, 7.5.2.2, 7.5.2.3, 7.5.2.4, 7.5.2.5,
7.5.2.6, 7.5.2.7, 7.5.2.8, 7.5.2.9, 7.5.2.10, 7.5.3.1, 7.5.3.2, 7.5.3.3, 7.5.3.4, 7.5.3.5, 7.5.3.6,
7.5.3.7, 7.5.3.8, 7.5.3.9, 7.5.3.10, 7.5.4.1, 7.5.4.2, 7.5.4.3, 7.5.4.4, 7.5.4.5, 7.5.4.6, 7.5.4.7,
7.5.4.8, 7.5.4.9, 7.5.4.10, 7.5.5.1, 7.5.5.2, 7.5.5.3, 7.5.5.4, 7.5.5.5, 7.5.5.6, 7.5.5.7, 7.5.5.8,
7.5.5.9, 7.5.5.10, 7.5.6.1, 7.5.6.2, 7.5.6.3, 7.5.6.4, 7.5.6.5, 7.5.6.6, 7.5.6.7, 7.5.6.8, 7.5.6.9,
7.5.6.10, 7.5.7.1, 7.5.7.2, 7.5.7.3, 7.5.7.4, 7.5.7.5, 7.5.7.6, 7.5.7.7, 7.5.7.8, 7.5.7.9, 7.5.7.10,
7.5.8.1, 7.5.8.2, 7.5.8.3, 7.5.8.4, 7.5.8.5, 7.5.8.6, 7.5.8.7, 7.5.8.8, 7.5.8.9, 7.5.8.10, 7.5.9.1,
7.5.9.2, 7.5.9.3, 7.5.9.4, 7.5.9.5, 7.5.9.6, 7.5.9.7, 7.5.9.8, 7.5.9.9, 7.5.9.10, 7.5.10.1, 7.5.10.2,
7.5.10.3, 7.5.10.4, 7.5.10.5, 7.5.10.6, 7.5.10.7, 7.5.10.8, 7.5.10.9, 7.5.10.10, 7.6.1.1, 7.6.1.2,
7.6.1.3, 7.6.1.4, 7.6.1.5, 7.6.1.6, 7.6.1.7, 7.6.1.8, 7.6.1.9, 7.6.1.10, 7.6.2.1, 7.6.2.2, 7.6.2.3,
7.6.2.4, 7.6.2.5, 7.6.2.6, 7.6.2.7, 7.6.2.8, 7.6.2.9, 7.6.2.10, 7.6.3.1, 7.6.3.2, 7.6.3.3, 7.6.3.4,
7.6.3.5, 7.6.3.6, 7.6.3.7, 7.6.3.8, 7.6.3.9, 7.6.3.10, 7.6.4.1, 7.6.4.2, 7.6.4.3, 7.6.4.4, 7.6.4.5,
7.6.4.6, 7.6.4.7, 7.6.4.8, 7.6.4.9, 7.6.4.10, 7.6.5.1, 7.6.5.2, 7.6.5.3, 7.6.5.4, 7.6.5.5, 7.6.5.6,
7.6.5.7, 7.6.5.8, 7.6.5.9, 7.6.5.10, 7.6.6.1, 7.6.6.2, 7.6.6.3, 7.6.6.4, 7.6.6.5, 7.6.6.6, 7.6.6.7,
7.6.6.8, 7.6.6.9, 7.6.6.10, 7.6.7.1, 7.6.7.2, 7.6.7.3, 7.6.7.4, 7.6.7.5, 7.6.7.6, 7.6.7.7, 7.6.7.8,
7.6.7.9, 7.6.7.10, 7.6.8.1, 7.6.8.2, 7.6.8.3, 7.6.8.4, 7.6.8.5, 7.6.8.6, 7.6.8.7, 7.6.8.8, 7.6.8.9,
7.6.8.10, 7.6.9.1, 7.6.9.2, 7.6.9.3, 7.6.9.4, 7.6.9.5, 7.6.9.6, 7.6.9.7, 7.6.9.8, 7.6.9.9, 7.6.9.10,
7.6.10.1, 7.6.10.2, 7.6.10.3, 7.6.10.4, 7.6.10.5, 7.6.10.6, 7.6.10.7, 7.6.10.8, 7.6.10.9, 7.6.10.10,
7.7.1.1, 7.7.1.2, 7.7.1.3, 7.7.1.4, 7.7.1.5, 7.7.1.6, 7.7.1.7, 7.7.1.8, 7.7.1.9, 7.7.1.10, 7.7.2.1,
7.7.2.2, 7.7.2.3, 7.7.2.4, 7.7.2.5, 7.7.2.6, 7.7.2.7, 7.7.2.8, 7.7.2.9, 7.7.2.10, 7.7.3.1, 7.7.3.2,
7.7.3.3, 7.7.3.4, 7.7.3.5, 7.7.3.6, 7.7.3.7, 7.7.3.8, 7.7.3.9, 7.7.3.10, 7.7.4.1, 7.7.4.2, 7.7.4.3,
7.7.4.4, 7.7.4.5, 7.7.4.6, 7.7.4.7, 7.7.4.8, 7.7.4.9, 7.7.4.10, 7.7.5.1, 7.7.5.2, 7.7.5.3, 7.7.5.4,
7.7.5.5, 7.7.5.6, 7.7.5.7, 7.7.5.8, 7.7.5.9, 7.7.5.10, 7.7.6.1, 7.7.6.2, 7.7.6.3, 7.7.6.4, 7.7.6.5,
7.7.6.6, 7.7.6.7, 7.7.6.8, 7.7.6.9, 7.7.6.10, 7.7.7.1, 7.7.7.2, 7.7.7.3, 7.7.7.4, 7.7.7.5, 7.7.7.6,
7.7.7.7, 7.7.7.8, 7.7.7.9, 7.7.7.10, 7.7.8.1, 7.7.8.2, 7.7.8.3, 7.7.8.4, 7.7.8.5, 7.7.8.6, 7.7.8.7,
7.7.8.8, 7.7.8.9, 7.7.8.10, 7.7.9.1, 7.7.9.2, 7.7.9.3, 7.7.9.4, 7.7.9.5, 7.7.9.6, 7.7.9.7, 7.7.9.8,
7.7.9.9, 7.7.9.10, 7.7.10.1, 7.7.10.2, 7.7.10.3, 7.7.10.4, 7.7.10.5, 7.7.10.6, 7.7.10.7, 7.7.10.8,
7.7.10.9, 7.7.10.10, 7.8.1.1, 7.8.1.2, 7.8.1.3, 7.8.1.4, 7.8.1.5, 7.8.1.6, 7.8.1.7, 7.8.1.8, 7.8.1.9,
7.8.1.10, 7.8.2.1, 7.8.2.2, 7.8.2.3, 7.8.2.4, 7.8.2.5, 7.8.2.6, 7.8.2.7, 7.8.2.8, 7.8.2.9, 7.8.2.10,
7.8.3.1, 7.8.3.2, 7.8.3.3, 7.8.3.4, 7.8.3.5, 7.8.3.6, 7.8.3.7, 7.8.3.8, 7.8.3.9, 7.8.3.10, 7.8.4.1,
7.8.4.2, 7.8.4.3, 7.8.4.4, 7.8.4.5, 7.8.4.6, 7.8.4.7, 7.8.4.8, 7.8.4.9, 7.8.4.10, 7.8.5.1, 7.8.5.2,
7.8.5.3, 7.8.5.4, 7.8.5.5, 7.8.5.6, 7.8.5.7, 7.8.5.8, 7.8.5.9, 7.8.5.10, 7.8.6.1, 7.8.6.2, 7.8.6.3,
7.8.6.4, 7.8.6.5, 7.8.6.6, 7.8.6.7, 7.8.6.8, 7.8.6.9, 7.8.6.10, 7.8.7.1, 7.8.7.2, 7.8.7.3, 7.8.7.4,
7.8.7.5, 7.8.7.6, 7.8.7.7, 7.8.7.8, 7.8.7.9, 7.8.7.10, 7.8.8.1, 7.8.8.2, 7.8.8.3, 7.8.8.4, 7.8.8.5,
7.8.8.6, 7.8.8.7, 7.8.8.8, 7.8.8.9, 7.8.8.10, 7.8.9.1, 7.8.9.2, 7.8.9.3, 7.8.9.4, 7.8.9.5, 7.8.9.6,
7.8.9.7, 7.8.9.8, 7.8.9.9, 7.8.9.10, 7.8.10.1, 7.8.10.2, 7.8.10.3, 7.8.10.4, 7.8.10.5, 7.8.10.6,
7.8.10.7, 7.8.10.8, 7.8.10.9, 7.8.10.10, 7.9.1.1, 7.9.1.2, 7.9.1.3, 7.9.1.4, 7.9.1.5, 7.9.1.6, 7.9.1.7,
7.9.1.8, 7.9.1.9, 7.9.1.10, 7.9.2.1, 7.9.2.2, 7.9.2.3, 7.9.2.4, 7.9.2.5, 7.9.2.6, 7.9.2.7, 7.9.2.8,
7.9.2.9, 7.9.2.10, 7.9.3.1, 7.9.3.2, 7.9.3.3, 7.9.3.4, 7.9.3.5, 7.9.3.6, 7.9.3.7, 7.9.3.8, 7.9.3.9,
7.9.3.10, 7.9.4.1, 7.9.4.2, 7.9.4.3, 7.9.4.4, 7.9.4.5, 7.9.4.6, 7.9.4.7, 7.9.4.8, 7.9.4.9, 7.9.4.10,
7.9.5.1, 7.9.5.2, 7.9.5.3, 7.9.5.4, 7.9.5.5, 7.9.5.6, 7.9.5.7, 7.9.5.8, 7.9.5.9, 7.9.5.10, 7.9.6.1,
7.9.6.2, 7.9.6.3, 7.9.6.4, 7.9.6.5, 7.9.6.6, 7.9.6.7, 7.9.6.8, 7.9.6.9, 7.9.6.10, 7.9.7.1, 7.9.7.2,
7.9.7.3, 7.9.7.4, 7.9.7.5, 7.9.7.6, 7.9.7.7, 7.9.7.8, 7.9.7.9, 7.9.7.10, 7.9.8.1, 7.9.8.2, 7.9.8.3,
7.9.8.4, 7.9.8.5, 7.9.8.6, 7.9.8.7, 7.9.8.8, 7.9.8.9, 7.9.8.10, 7.9.9.1, 7.9.9.2, 7.9.9.3, 7.9.9.4,
7.9.9.5, 7.9.9.6, 7.9.9.7, 7.9.9.8, 7.9.9.9, 7.9.9.10, 7.9.10.1, 7.9.10.2, 7.9.10.3, 7.9.10.4, 7.9.10.5,
7.9.10.6, 7.9.10.7, 7.9.10.8, 7.9.10.9, 7.9.10.10, 7.10.1.1, 7.10.1.2, 7.10.1.3, 7.10.1.4, 7.10.1.5,
7.10.1.6, 7.10.1.7, 7.10.1.8, 7.10.1.9, 7.10.1.10, 7.10.2.1, 7.10.2.2, 7.10.2.3, 7.10.2.4, 7.10.2.5,
7.10.2.6, 7.10.2.7, 7.10.2.8, 7.10.2.9, 7.10.2.10, 7.10.3.1, 7.10.3.2, 7.10.3.3, 7.10.3.4, 7.10.3.5,
7.10.3.6, 7.10.3.7, 7.10.3.8, 7.10.3.9, 7.10.3.10, 7.10.4.1, 7.10.4.2, 7.10.4.3, 7.10.4.4, 7.10.4.5,
7.10.4.6, 7.10.4.7, 7.10.4.8, 7.10.4.9, 7.10.4.10, 7.10.5.1, 7.10.5.2, 7.10.5.3, 7.10.5.4, 7.10.5.5,
7.10.5.6, 7.10.5.7, 7.10.5.8, 7.10.5.9, 7.10.5.10, 7.10.6.1, 7.10.6.2, 7.10.6.3, 7.10.6.4, 7.10.6.5,
7.10.6.6, 7.10.6.7, 7.10.6.8, 7.10.6.9, 7.10.6.10, 7.10.7.1, 7.10.7.2, 7.10.7.3, 7.10.7.4, 7.10.7.5,
7.10.7.6, 7.10.7.7, 7.10.7.8, 7.10.7.9, 7.10.7.10, 7.10.8.1, 7.10.8.2, 7.10.8.3, 7.10.8.4, 7.10.8.5,
7.10.8.6, 7.10.8.7, 7.10.8.8, 7.10.8.9, 7.10.8.10, 7.10.9.1, 7.10.9.2, 7.10.9.3, 7.10.9.4, 7.10.9.5,
7.10.9.6, 7.10.9.7, 7.10.9.8, 7.10.9.9, 7.10.9.10, 7.10.10.1, 7.10.10.2, 7.10.10.3, 7.10.10.4,
7.10.10.5, 7.10.10.6, 7.10.10.7, 7.10.10.8, 7.10.10.9, 7.10.10.10, 8.1.1.1, 8.1.1.2, 8.1.1.3, 8.1.1.4,
8.1.1.5, 8.1.1.6, 8.1.1.7, 8.1.1.8, 8.1.1.9, 8.1.1.10, 8.1.2.1, 8.1.2.2, 8.1.2.3, 8.1.2.4, 8.1.2.5,
8.1.2.6, 8.1.2.7, 8.1.2.8, 8.1.2.9, 8.1.2.10, 8.1.3.1, 8.1.3.2, 8.1.3.3, 8.1.3.4, 8.1.3.5, 8.1.3.6,
8.1.3.7, 8.1.3.8, 8.1.3.9, 8.1.3.10, 8.1.4.1, 8.1.4.2, 8.1.4.3, 8.1.4.4, 8.1.4.5, 8.1.4.6, 8.1.4.7,
8.1.4.8, 8.1.4.9, 8.1.4.10, 8.1.5.1, 8.1.5.2, 8.1.5.3, 8.1.5.4, 8.1.5.5, 8.1.5.6, 8.1.5.7, 8.1.5.8,
8.1.5.9, 8.1.5.10, 8.1.6.1, 8.1.6.2, 8.1.6.3, 8.1.6.4, 8.1.6.5, 8.1.6.6, 8.1.6.7, 8.1.6.8, 8.1.6.9,
8.1.6.10, 8.1.7.1, 8.1.7.2, 8.1.7.3, 8.1.7.4, 8.1.7.5, 8.1.7.6, 8.1.7.7, 8.1.7.8, 8.1.7.9, 8.1.7.10,
8.1.8.1, 8.1.8.2, 8.1.8.3, 8.1.8.4, 8.1.8.5, 8.1.8.6, 8.1.8.7, 8.1.8.8, 8.1.8.9, 8.1.8.10, 8.1.9.1,
8.1.9.2, 8.1.9.3, 8.1.9.4, 8.1.9.5, 8.1.9.6, 8.1.9.7, 8.1.9.8, 8.1.9.9, 8.1.9.10, 8.1.10.1, 8.1.10.2,
8.1.10.3, 8.1.10.4, 8.1.10.5, 8.1.10.6, 8.1.10.7, 8.1.10.8, 8.1.10.9, 8.1.10.10, 8.2.1.1, 8.2.1.2,
8.2.1.3, 8.2.1.4, 8.2.1.5, 8.2.1.6, 8.2.1.7, 8.2.1.8, 8.2.1.9, 8.2.1.10, 8.2.2.1, 8.2.2.2, 8.2.2.3,
8.2.2.4, 8.2.2.5, 8.2.2.6, 8.2.2.7, 8.2.2.8, 8.2.2.9, 8.2.2.10, 8.2.3.1, 8.2.3.2, 8.2.3.3, 8.2.3.4,
8.2.3.5, 8.2.3.6, 8.2.3.7, 8.2.3.8, 8.2.3.9, 8.2.3.10, 8.2.4.1, 8.2.4.2, 8.2.4.3, 8.2.4.4, 8.2.4.5,
8.2.4.6, 8.2.4.7, 8.2.4.8, 8.2.4.9, 8.2.4.10, 8.2.5.1, 8.2.5.2, 8.2.5.3, 8.2.5.4, 8.2.5.5, 8.2.5.6,
8.2.5.7, 8.2.5.8, 8.2.5.9, 8.2.5.10, 8.2.6.1, 8.2.6.2, 8.2.6.3, 8.2.6.4, 8.2.6.5, 8.2.6.6, 8.2.6.7,
8.2.6.8, 8.2.6.9, 8.2.6.10, 8.2.7.1, 8.2.7.2, 8.2.7.3, 8.2.7.4, 8.2.7.5, 8.2.7.6, 8.2.7.7, 8.2.7.8,
8.2.7.9, 8.2.7.10, 8.2.8.1, 8.2.8.2, 8.2.8.3, 8.2.8.4, 8.2.8.5, 8.2.8.6, 8.2.8.7, 8.2.8.8, 8.2.8.9,
8.2.8.10, 8.2.9.1, 8.2.9.2, 8.2.9.3, 8.2.9.4, 8.2.9.5, 8.2.9.6, 8.2.9.7, 8.2.9.8, 8.2.9.9, 8.2.9.10,
8.2.10.1, 8.2.10.2, 8.2.10.3, 8.2.10.4, 8.2.10.5, 8.2.10.6, 8.2.10.7, 8.2.10.8, 8.2.10.9, 8.2.10.10,
8.3.1.1, 8.3.1.2, 8.3.1.3, 8.3.1.4, 8.3.1.5, 8.3.1.6, 8.3.1.7, 8.3.1.8, 8.3.1.9, 8.3.1.10, 8.3.2.1,
8.3.2.2, 8.3.2.3, 8.3.2.4, 8.3.2.5, 8.3.2.6, 8.3.2.7, 8.3.2.8, 8.3.2.9, 8.3.2.10, 8.3.3.1, 8.3.3.2,
8.3.3.3, 8.3.3.4, 8.3.3.5, 8.3.3.6, 8.3.3.7, 8.3.3.8, 8.3.3.9, 8.3.3.10, 8.3.4.1, 8.3.4.2, 8.3.4.3,
8.3.4.4, 8.3.4.5, 8.3.4.6, 8.3.4.7, 8.3.4.8, 8.3.4.9, 8.3.4.10, 8.3.5.1, 8.3.5.2, 8.3.5.3, 8.3.5.4,
8.3.5.5, 8.3.5.6, 8.3.5.7, 8.3.5.8, 8.3.5.9, 8.3.5.10, 8.3.6.1, 8.3.6.2, 8.3.6.3, 8.3.6.4, 8.3.6.5,
8.3.6.6, 8.3.6.7, 8.3.6.8, 8.3.6.9, 8.3.6.10, 8.3.7.1, 8.3.7.2, 8.3.7.3, 8.3.7.4, 8.3.7.5, 8.3.7.6,
8.3.7.7, 8.3.7.8, 8.3.7.9, 8.3.7.10, 8.3.8.1, 8.3.8.2, 8.3.8.3, 8.3.8.4, 8.3.8.5, 8.3.8.6, 8.3.8.7,
8.3.8.8, 8.3.8.9, 8.3.8.10, 8.3.9.1, 8.3.9.2, 8.3.9.3, 8.3.9.4, 8.3.9.5, 8.3.9.6, 8.3.9.7, 8.3.9.8,
8.3.9.9, 8.3.9.10, 8.3.10.1, 8.3.10.2, 8.3.10.3, 8.3.10.4, 8.3.10.5, 8.3.10.6, 8.3.10.7, 8.3.10.8,
8.3.10.9, 8.3.10.10, 8.4.1.1, 8.4.1.2, 8.4.1.3, 8.4.1.4, 8.4.1.5, 8.4.1.6, 8.4.1.7, 8.4.1.8, 8.4.1.9,
8.4.1.10, 8.4.2.1, 8.4.2.2, 8.4.2.3, 8.4.2.4, 8.4.2.5, 8.4.2.6, 8.4.2.7, 8.4.2.8, 8.4.2.9, 8.4.2.10,
8.4.3.1, 8.4.3.2, 8.4.3.3, 8.4.3.4, 8.4.3.5, 8.4.3.6, 8.4.3.7, 8.4.3.8, 8.4.3.9, 8.4.3.10, 8.4.4.1,
8.4.4.2, 8.4.4.3, 8.4.4.4, 8.4.4.5, 8.4.4.6, 8.4.4.7, 8.4.4.8, 8.4.4.9, 8.4.4.10, 8.4.5.1, 8.4.5.2,
8.4.5.3, 8.4.5.4, 8.4.5.5, 8.4.5.6, 8.4.5.7, 8.4.5.8, 8.4.5.9, 8.4.5.10, 8.4.6.1, 8.4.6.2, 8.4.6.3,
8.4.6.4, 8.4.6.5, 8.4.6.6, 8.4.6.7, 8.4.6.8, 8.4.6.9, 8.4.6.10, 8.4.7.1, 8.4.7.2, 8.4.7.3, 8.4.7.4,
8.4.7.5, 8.4.7.6, 8.4.7.7, 8.4.7.8, 8.4.7.9, 8.4.7.10, 8.4.8.1, 8.4.8.2, 8.4.8.3, 8.4.8.4, 8.4.8.5,
8.4.8.6, 8.4.8.7, 8.4.8.8, 8.4.8.9, 8.4.8.10, 8.4.9.1, 8.4.9.2, 8.4.9.3, 8.4.9.4, 8.4.9.5, 8.4.9.6,
8.4.9.7, 8.4.9.8, 8.4.9.9, 8.4.9.10, 8.4.10.1, 8.4.10.2, 8.4.10.3, 8.4.10.4, 8.4.10.5, 8.4.10.6,
8.4.10.7, 8.4.10.8, 8.4.10.9, 8.4.10.10, 8.5.1.1, 8.5.1.2, 8.5.1.3, 8.5.1.4, 8.5.1.5, 8.5.1.6, 8.5.1.7,
8.5.1.8, 8.5.1.9, 8.5.1.10, 8.5.2.1, 8.5.2.2, 8.5.2.3, 8.5.2.4, 8.5.2.5, 8.5.2.6, 8.5.2.7, 8.5.2.8,
8.5.2.9, 8.5.2.10, 8.5.3.1, 8.5.3.2, 8.5.3.3, 8.5.3.4, 8.5.3.5, 8.5.3.6, 8.5.3.7, 8.5.3.8, 8.5.3.9,
8.5.3.10, 8.5.4.1, 8.5.4.2, 8.5.4.3, 8.5.4.4, 8.5.4.5, 8.5.4.6, 8.5.4.7, 8.5.4.8, 8.5.4.9, 8.5.4.10,
8.5.5.1, 8.5.5.2, 8.5.5.3, 8.5.5.4, 8.5.5.5, 8.5.5.6, 8.5.5.7, 8.5.5.8, 8.5.5.9, 8.5.5.10, 8.5.6.1,
8.5.6.2, 8.5.6.3, 8.5.6.4, 8.5.6.5, 8.5.6.6, 8.5.6.7, 8.5.6.8, 8.5.6.9, 8.5.6.10, 8.5.7.1, 8.5.7.2,
8.5.7.3, 8.5.7.4, 8.5.7.5, 8.5.7.6, 8.5.7.7, 8.5.7.8, 8.5.7.9, 8.5.7.10, 8.5.8.1, 8.5.8.2, 8.5.8.3,
8.5.8.4, 8.5.8.5, 8.5.8.6, 8.5.8.7, 8.5.8.8, 8.5.8.9, 8.5.8.10, 8.5.9.1, 8.5.9.2, 8.5.9.3, 8.5.9.4,
8.5.9.5, 8.5.9.6, 8.5.9.7, 8.5.9.8, 8.5.9.9, 8.5.9.10, 8.5.10.1, 8.5.10.2, 8.5.10.3, 8.5.10.4, 8.5.10.5,
8.5.10.6, 8.5.10.7, 8.5.10.8, 8.5.10.9, 8.5.10.10, 8.6.1.1, 8.6.1.2, 8.6.1.3, 8.6.1.4, 8.6.1.5, 8.6.1.6,
8.6.1.7, 8.6.1.8, 8.6.1.9, 8.6.1.10, 8.6.2.1, 8.6.2.2, 8.6.2.3, 8.6.2.4, 8.6.2.5, 8.6.2.6, 8.6.2.7,
8.6.2.8, 8.6.2.9, 8.6.2.10, 8.6.3.1, 8.6.3.2, 8.6.3.3, 8.6.3.4, 8.6.3.5, 8.6.3.6, 8.6.3.7, 8.6.3.8,
8.6.3.9, 8.6.3.10, 8.6.4.1, 8.6.4.2, 8.6.4.3, 8.6.4.4, 8.6.4.5, 8.6.4.6, 8.6.4.7, 8.6.4.8, 8.6.4.9,
8.6.4.10, 8.6.5.1, 8.6.5.2, 8.6.5.3, 8.6.5.4, 8.6.5.5, 8.6.5.6, 8.6.5.7, 8.6.5.8, 8.6.5.9, 8.6.5.10,
8.6.6.1, 8.6.6.2, 8.6.6.3, 8.6.6.4, 8.6.6.5, 8.6.6.6, 8.6.6.7, 8.6.6.8, 8.6.6.9, 8.6.6.10, 8.6.7.1,
8.6.7.2, 8.6.7.3, 8.6.7.4, 8.6.7.5, 8.6.7.6, 8.6.7.7, 8.6.7.8, 8.6.7.9, 8.6.7.10, 8.6.8.1, 8.6.8.2,
8.6.8.3, 8.6.8.4, 8.6.8.5, 8.6.8.6, 8.6.8.7, 8.6.8.8, 8.6.8.9, 8.6.8.10, 8.6.9.1, 8.6.9.2, 8.6.9.3,
8.6.9.4, 8.6.9.5, 8.6.9.6, 8.6.9.7, 8.6.9.8, 8.6.9.9, 8.6.9.10, 8.6.10.1, 8.6.10.2, 8.6.10.3, 8.6.10.4,
8.6.10.5, 8.6.10.6, 8.6.10.7, 8.6.10.8, 8.6.10.9, 8.6.10.10, 8.7.1.1, 8.7.1.2, 8.7.1.3, 8.7.1.4,
8.7.1.5, 8.7.1.6, 8.7.1.7, 8.7.1.8, 8.7.1.9, 8.7.1.10, 8.7.2.1, 8.7.2.2, 8.7.2.3, 8.7.2.4, 8.7.2.5,
8.7.2.6, 8.7.2.7, 8.7.2.8, 8.7.2.9, 8.7.2.10, 8.7.3.1, 8.7.3.2, 8.7.3.3, 8.7.3.4, 8.7.3.5, 8.7.3.6,
8.7.3.7, 8.7.3.8, 8.7.3.9, 8.7.3.10, 8.7.4.1, 8.7.4.2, 8.7.4.3, 8.7.4.4, 8.7.4.5, 8.7.4.6, 8.7.4.7,
8.7.4.8, 8.7.4.9, 8.7.4.10, 8.7.5.1, 8.7.5.2, 8.7.5.3, 8.7.5.4, 8.7.5.5, 8.7.5.6, 8.7.5.7, 8.7.5.8,
8.7.5.9, 8.7.5.10, 8.7.6.1, 8.7.6.2, 8.7.6.3, 8.7.6.4, 8.7.6.5, 8.7.6.6, 8.7.6.7, 8.7.6.8, 8.7.6.9,
8.7.6.10, 8.7.7.1, 8.7.7.2, 8.7.7.3, 8.7.7.4, 8.7.7.5, 8.7.7.6, 8.7.7.7, 8.7.7.8, 8.7.7.9, 8.7.7.10,
8.7.8.1, 8.7.8.2, 8.7.8.3, 8.7.8.4, 8.7.8.5, 8.7.8.6, 8.7.8.7, 8.7.8.8, 8.7.8.9, 8.7.8.10, 8.7.9.1,
8.7.9.2, 8.7.9.3, 8.7.9.4, 8.7.9.5, 8.7.9.6, 8.7.9.7, 8.7.9.8, 8.7.9.9, 8.7.9.10, 8.7.10.1, 8.7.10.2,
8.7.10.3, 8.7.10.4, 8.7.10.5, 8.7.10.6, 8.7.10.7, 8.7.10.8, 8.7.10.9, 8.7.10.10, 8.8.1.1, 8.8.1.2,
8.8.1.3, 8.8.1.4, 8.8.1.5, 8.8.1.6, 8.8.1.7, 8.8.1.8, 8.8.1.9, 8.8.1.10, 8.8.2.1, 8.8.2.2, 8.8.2.3,
8.8.2.4, 8.8.2.5, 8.8.2.6, 8.8.2.7, 8.8.2.8, 8.8.2.9, 8.8.2.10, 8.8.3.1, 8.8.3.2, 8.8.3.3, 8.8.3.4,
8.8.3.5, 8.8.3.6, 8.8.3.7, 8.8.3.8, 8.8.3.9, 8.8.3.10, 8.8.4.1, 8.8.4.2, 8.8.4.3, 8.8.4.4, 8.8.4.5,
8.8.4.6, 8.8.4.7, 8.8.4.8, 8.8.4.9, 8.8.4.10, 8.8.5.1, 8.8.5.2, 8.8.5.3, 8.8.5.4, 8.8.5.5, 8.8.5.6,
8.8.5.7, 8.8.5.8, 8.8.5.9, 8.8.5.10, 8.8.6.1, 8.8.6.2, 8.8.6.3, 8.8.6.4, 8.8.6.5, 8.8.6.6, 8.8.6.7,
8.8.6.8, 8.8.6.9, 8.8.6.10, 8.8.7.1, 8.8.7.2, 8.8.7.3, 8.8.7.4, 8.8.7.5, 8.8.7.6, 8.8.7.7, 8.8.7.8,
8.8.7.9, 8.8.7.10, 8.8.8.1, 8.8.8.2, 8.8.8.3, 8.8.8.4, 8.8.8.5, 8.8.8.6, 8.8.8.7, 8.8.8.8, 8.8.8.9,
8.8.8.10, 8.8.9.1, 8.8.9.2, 8.8.9.3, 8.8.9.4, 8.8.9.5, 8.8.9.6, 8.8.9.7, 8.8.9.8, 8.8.9.9, 8.8.9.10,
8.8.10.1, 8.8.10.2, 8.8.10.3, 8.8.10.4, 8.8.10.5, 8.8.10.6, 8.8.10.7, 8.8.10.8, 8.8.10.9, 8.8.10.10,
8.9.1.1, 8.9.1.2, 8.9.1.3, 8.9.1.4, 8.9.1.5, 8.9.1.6, 8.9.1.7, 8.9.1.8, 8.9.1.9, 8.9.1.10, 8.9.2.1,
8.9.2.2, 8.9.2.3, 8.9.2.4, 8.9.2.5, 8.9.2.6, 8.9.2.7, 8.9.2.8, 8.9.2.9, 8.9.2.10, 8.9.3.1, 8.9.3.2,
8.9.3.3, 8.9.3.4, 8.9.3.5, 8.9.3.6, 8.9.3.7, 8.9.3.8, 8.9.3.9, 8.9.3.10, 8.9.4.1, 8.9.4.2, 8.9.4.3,
8.9.4.4, 8.9.4.5, 8.9.4.6, 8.9.4.7, 8.9.4.8, 8.9.4.9, 8.9.4.10, 8.9.5.1, 8.9.5.2, 8.9.5.3, 8.9.5.4,
8.9.5.5, 8.9.5.6, 8.9.5.7, 8.9.5.8, 8.9.5.9, 8.9.5.10, 8.9.6.1, 8.9.6.2, 8.9.6.3, 8.9.6.4, 8.9.6.5,
8.9.6.6, 8.9.6.7, 8.9.6.8, 8.9.6.9, 8.9.6.10, 8.9.7.1, 8.9.7.2, 8.9.7.3, 8.9.7.4, 8.9.7.5, 8.9.7.6,
8.9.7.7, 8.9.7.8, 8.9.7.9, 8.9.7.10, 8.9.8.1, 8.9.8.2, 8.9.8.3, 8.9.8.4, 8.9.8.5, 8.9.8.6, 8.9.8.7,
8.9.8.8, 8.9.8.9, 8.9.8.10, 8.9.9.1, 8.9.9.2, 8.9.9.3, 8.9.9.4, 8.9.9.5, 8.9.9.6, 8.9.9.7, 8.9.9.8,
8.9.9.9, 8.9.9.10, 8.9.10.1, 8.9.10.2, 8.9.10.3, 8.9.10.4, 8.9.10.5, 8.9.10.6, 8.9.10.7, 8.9.10.8,
8.9.10.9, 8.9.10.10, 8.10.1.1, 8.10.1.2, 8.10.1.3, 8.10.1.4, 8.10.1.5, 8.10.1.6, 8.10.1.7, 8.10.1.8,
8.10.1.9, 8.10.1.10, 8.10.2.1, 8.10.2.2, 8.10.2.3, 8.10.2.4, 8.10.2.5, 8.10.2.6, 8.10.2.7, 8.10.2.8,
8.10.2.9, 8.10.2.10, 8.10.3.1, 8.10.3.2, 8.10.3.3, 8.10.3.4, 8.10.3.5, 8.10.3.6, 8.10.3.7, 8.10.3.8,
8.10.3.9, 8.10.3.10, 8.10.4.1, 8.10.4.2, 8.10.4.3, 8.10.4.4, 8.10.4.5, 8.10.4.6, 8.10.4.7, 8.10.4.8,
8.10.4.9, 8.10.4.10, 8.10.5.1, 8.10.5.2, 8.10.5.3, 8.10.5.4, 8.10.5.5, 8.10.5.6, 8.10.5.7, 8.10.5.8,
8.10.5.9, 8.10.5.10, 8.10.6.1, 8.10.6.2, 8.10.6.3, 8.10.6.4, 8.10.6.5, 8.10.6.6, 8.10.6.7, 8.10.6.8,
8.10.6.9, 8.10.6.10, 8.10.7.1, 8.10.7.2, 8.10.7.3, 8.10.7.4, 8.10.7.5, 8.10.7.6, 8.10.7.7, 8.10.7.8,
8.10.7.9, 8.10.7.10, 8.10.8.1, 8.10.8.2, 8.10.8.3, 8.10.8.4, 8.10.8.5, 8.10.8.6, 8.10.8.7, 8.10.8.8,
8.10.8.9, 8.10.8.10, 8.10.9.1, 8.10.9.2, 8.10.9.3, 8.10.9.4, 8.10.9.5, 8.10.9.6, 8.10.9.7, 8.10.9.8,
8.10.9.9, 8.10.9.10, 8.10.10.1, 8.10.10.2, 8.10.10.3, 8.10.10.4, 8.10.10.5, 8.10.10.6, 8.10.10.7,
8.10.10.8, 8.10.10.9, 8.10.10.10, 9.1.1.1, 9.1.1.2, 9.1.1.3, 9.1.1.4, 9.1.1.5, 9.1.1.6, 9.1.1.7,
9.1.1.8, 9.1.1.9, 9.1.1.10, 9.1.2.1, 9.1.2.2, 9.1.2.3, 9.1.2.4, 9.1.2.5, 9.1.2.6, 9.1.2.7, 9.1.2.8,
9.1.2.9, 9.1.2.10, 9.1.3.1, 9.1.3.2, 9.1.3.3, 9.1.3.4, 9.1.3.5, 9.1.3.6, 9.1.3.7, 9.1.3.8, 9.1.3.9,
9.1.3.10, 9.1.4.1, 9.1.4.2, 9.1.4.3, 9.1.4.4, 9.1.4.5, 9.1.4.6, 9.1.4.7, 9.1.4.8, 9.1.4.9, 9.1.4.10,
9.1.5.1, 9.1.5.2, 9.1.5.3, 9.1.5.4, 9.1.5.5, 9.1.5.6, 9.1.5.7, 9.1.5.8, 9.1.5.9, 9.1.5.10, 9.1.6.1,
9.1.6.2, 9.1.6.3, 9.1.6.4, 9.1.6.5, 9.1.6.6, 9.1.6.7, 9.1.6.8, 9.1.6.9, 9.1.6.10, 9.1.7.1, 9.1.7.2,
9.1.7.3, 9.1.7.4, 9.1.7.5, 9.1.7.6, 9.1.7.7, 9.1.7.8, 9.1.7.9, 9.1.7.10, 9.1.8.1, 9.1.8.2, 9.1.8.3,
9.1.8.4, 9.1.8.5, 9.1.8.6, 9.1.8.7, 9.1.8.8, 9.1.8.9, 9.1.8.10, 9.1.9.1, 9.1.9.2, 9.1.9.3, 9.1.9.4,
9.1.9.5, 9.1.9.6, 9.1.9.7, 9.1.9.8, 9.1.9.9, 9.1.9.10, 9.1.10.1, 9.1.10.2, 9.1.10.3, 9.1.10.4, 9.1.10.5,
9.1.10.6, 9.1.10.7, 9.1.10.8, 9.1.10.9, 9.1.10.10, 9.2.1.1, 9.2.1.2, 9.2.1.3, 9.2.1.4, 9.2.1.5, 9.2.1.6,
9.2.1.7, 9.2.1.8, 9.2.1.9, 9.2.1.10, 9.2.2.1, 9.2.2.2, 9.2.2.3, 9.2.2.4, 9.2.2.5, 9.2.2.6, 9.2.2.7,
9.2.2.8, 9.2.2.9, 9.2.2.10, 9.2.3.1, 9.2.3.2, 9.2.3.3, 9.2.3.4, 9.2.3.5, 9.2.3.6, 9.2.3.7, 9.2.3.8,
9.2.3.9, 9.2.3.10, 9.2.4.1, 9.2.4.2, 9.2.4.3, 9.2.4.4, 9.2.4.5, 9.2.4.6, 9.2.4.7, 9.2.4.8, 9.2.4.9,
9.2.4.10, 9.2.5.1, 9.2.5.2, 9.2.5.3, 9.2.5.4, 9.2.5.5, 9.2.5.6, 9.2.5.7, 9.2.5.8, 9.2.5.9, 9.2.5.10,
9.2.6.1, 9.2.6.2, 9.2.6.3, 9.2.6.4, 9.2.6.5, 9.2.6.6, 9.2.6.7, 9.2.6.8, 9.2.6.9, 9.2.6.10, 9.2.7.1,
9.2.7.2, 9.2.7.3, 9.2.7.4, 9.2.7.5, 9.2.7.6, 9.2.7.7, 9.2.7.8, 9.2.7.9, 9.2.7.10, 9.2.8.1, 9.2.8.2,
9.2.8.3, 9.2.8.4, 9.2.8.5, 9.2.8.6, 9.2.8.7, 9.2.8.8, 9.2.8.9, 9.2.8.10, 9.2.9.1, 9.2.9.2, 9.2.9.3,
9.2.9.4, 9.2.9.5, 9.2.9.6, 9.2.9.7, 9.2.9.8, 9.2.9.9, 9.2.9.10, 9.2.10.1, 9.2.10.2, 9.2.10.3, 9.2.10.4,
9.2.10.5, 9.2.10.6, 9.2.10.7, 9.2.10.8, 9.2.10.9, 9.2.10.10, 9.3.1.1, 9.3.1.2, 9.3.1.3, 9.3.1.4,
9.3.1.5, 9.3.1.6, 9.3.1.7, 9.3.1.8, 9.3.1.9, 9.3.1.10, 9.3.2.1, 9.3.2.2, 9.3.2.3, 9.3.2.4, 9.3.2.5,
9.3.2.6, 9.3.2.7, 9.3.2.8, 9.3.2.9, 9.3.2.10, 9.3.3.1, 9.3.3.2, 9.3.3.3, 9.3.3.4, 9.3.3.5, 9.3.3.6,
9.3.3.7, 9.3.3.8, 9.3.3.9, 9.3.3.10, 9.3.4.1, 9.3.4.2, 9.3.4.3, 9.3.4.4, 9.3.4.5, 9.3.4.6, 9.3.4.7,
9.3.4.8, 9.3.4.9, 9.3.4.10, 9.3.5.1, 9.3.5.2, 9.3.5.3, 9.3.5.4, 9.3.5.5, 9.3.5.6, 9.3.5.7, 9.3.5.8,
9.3.5.9, 9.3.5.10, 9.3.6.1, 9.3.6.2, 9.3.6.3, 9.3.6.4, 9.3.6.5, 9.3.6.6, 9.3.6.7, 9.3.6.8, 9.3.6.9,
9.3.6.10, 9.3.7.1, 9.3.7.2, 9.3.7.3, 9.3.7.4, 9.3.7.5, 9.3.7.6, 9.3.7.7, 9.3.7.8, 9.3.7.9, 9.3.7.10,
9.3.8.1, 9.3.8.2, 9.3.8.3, 9.3.8.4, 9.3.8.5, 9.3.8.6, 9.3.8.7, 9.3.8.8, 9.3.8.9, 9.3.8.10, 9.3.9.1,
9.3.9.2, 9.3.9.3, 9.3.9.4, 9.3.9.5, 9.3.9.6, 9.3.9.7, 9.3.9.8, 9.3.9.9, 9.3.9.10, 9.3.10.1, 9.3.10.2,
9.3.10.3, 9.3.10.4, 9.3.10.5, 9.3.10.6, 9.3.10.7, 9.3.10.8, 9.3.10.9, 9.3.10.10, 9.4.1.1, 9.4.1.2,
9.4.1.3, 9.4.1.4, 9.4.1.5, 9.4.1.6, 9.4.1.7, 9.4.1.8, 9.4.1.9, 9.4.1.10, 9.4.2.1, 9.4.2.2, 9.4.2.3,
9.4.2.4, 9.4.2.5, 9.4.2.6, 9.4.2.7, 9.4.2.8, 9.4.2.9, 9.4.2.10, 9.4.3.1, 9.4.3.2, 9.4.3.3, 9.4.3.4,
9.4.3.5, 9.4.3.6, 9.4.3.7, 9.4.3.8, 9.4.3.9, 9.4.3.10, 9.4.4.1, 9.4.4.2, 9.4.4.3, 9.4.4.4, 9.4.4.5,
9.4.4.6, 9.4.4.7, 9.4.4.8, 9.4.4.9, 9.4.4.10, 9.4.5.1, 9.4.5.2, 9.4.5.3, 9.4.5.4, 9.4.5.5, 9.4.5.6,
9.4.5.7, 9.4.5.8, 9.4.5.9, 9.4.5.10, 9.4.6.1, 9.4.6.2, 9.4.6.3, 9.4.6.4, 9.4.6.5, 9.4.6.6, 9.4.6.7,
9.4.6.8, 9.4.6.9, 9.4.6.10, 9.4.7.1, 9.4.7.2, 9.4.7.3, 9.4.7.4, 9.4.7.5, 9.4.7.6, 9.4.7.7, 9.4.7.8,
9.4.7.9, 9.4.7.10, 9.4.8.1, 9.4.8.2, 9.4.8.3, 9.4.8.4, 9.4.8.5, 9.4.8.6, 9.4.8.7, 9.4.8.8, 9.4.8.9,
9.4.8.10, 9.4.9.1, 9.4.9.2, 9.4.9.3, 9.4.9.4, 9.4.9.5, 9.4.9.6, 9.4.9.7, 9.4.9.8, 9.4.9.9, 9.4.9.10,
9.4.10.1, 9.4.10.2, 9.4.10.3, 9.4.10.4, 9.4.10.5, 9.4.10.6, 9.4.10.7, 9.4.10.8, 9.4.10.9, 9.4.10.10,
9.5.1.1, 9.5.1.2, 9.5.1.3, 9.5.1.4, 9.5.1.5, 9.5.1.6, 9.5.1.7, 9.5.1.8, 9.5.1.9, 9.5.1.10, 9.5.2.1,
9.5.2.2, 9.5.2.3, 9.5.2.4, 9.5.2.5, 9.5.2.6, 9.5.2.7, 9.5.2.8, 9.5.2.9, 9.5.2.10, 9.5.3.1, 9.5.3.2,
9.5.3.3, 9.5.3.4, 9.5.3.5, 9.5.3.6, 9.5.3.7, 9.5.3.8, 9.5.3.9, 9.5.3.10, 9.5.4.1, 9.5.4.2, 9.5.4.3,
9.5.4.4, 9.5.4.5, 9.5.4.6, 9.5.4.7, 9.5.4.8, 9.5.4.9, 9.5.4.10, 9.5.5.1, 9.5.5.2, 9.5.5.3, 9.5.5.4,
9.5.5.5, 9.5.5.6, 9.5.5.7, 9.5.5.8, 9.5.5.9, 9.5.5.10, 9.5.6.1, 9.5.6.2, 9.5.6.3, 9.5.6.4, 9.5.6.5,
9.5.6.6, 9.5.6.7, 9.5.6.8, 9.5.6.9, 9.5.6.10, 9.5.7.1, 9.5.7.2, 9.5.7.3, 9.5.7.4, 9.5.7.5, 9.5.7.6,
9.5.7.7, 9.5.7.8, 9.5.7.9, 9.5.7.10, 9.5.8.1, 9.5.8.2, 9.5.8.3, 9.5.8.4, 9.5.8.5, 9.5.8.6, 9.5.8.7,
9.5.8.8, 9.5.8.9, 9.5.8.10, 9.5.9.1, 9.5.9.2, 9.5.9.3, 9.5.9.4, 9.5.9.5, 9.5.9.6, 9.5.9.7, 9.5.9.8,
9.5.9.9, 9.5.9.10, 9.5.10.1, 9.5.10.2, 9.5.10.3, 9.5.10.4, 9.5.10.5, 9.5.10.6, 9.5.10.7, 9.5.10.8,
9.5.10.9, 9.5.10.10, 9.6.1.1, 9.6.1.2, 9.6.1.3, 9.6.1.4, 9.6.1.5, 9.6.1.6, 9.6.1.7, 9.6.1.8, 9.6.1.9,
9.6.1.10, 9.6.2.1, 9.6.2.2, 9.6.2.3, 9.6.2.4, 9.6.2.5, 9.6.2.6, 9.6.2.7, 9.6.2.8, 9.6.2.9, 9.6.2.10,
9.6.3.1, 9.6.3.2, 9.6.3.3, 9.6.3.4, 9.6.3.5, 9.6.3.6, 9.6.3.7, 9.6.3.8, 9.6.3.9, 9.6.3.10, 9.6.4.1,
9.6.4.2, 9.6.4.3, 9.6.4.4, 9.6.4.5, 9.6.4.6, 9.6.4.7, 9.6.4.8, 9.6.4.9, 9.6.4.10, 9.6.5.1, 9.6.5.2,
9.6.5.3, 9.6.5.4, 9.6.5.5, 9.6.5.6, 9.6.5.7, 9.6.5.8, 9.6.5.9, 9.6.5.10, 9.6.6.1, 9.6.6.2, 9.6.6.3,
9.6.6.4, 9.6.6.5, 9.6.6.6, 9.6.6.7, 9.6.6.8, 9.6.6.9, 9.6.6.10, 9.6.7.1, 9.6.7.2, 9.6.7.3, 9.6.7.4,
9.6.7.5, 9.6.7.6, 9.6.7.7, 9.6.7.8, 9.6.7.9, 9.6.7.10, 9.6.8.1, 9.6.8.2, 9.6.8.3, 9.6.8.4, 9.6.8.5,
9.6.8.6, 9.6.8.7, 9.6.8.8, 9.6.8.9, 9.6.8.10, 9.6.9.1, 9.6.9.2, 9.6.9.3, 9.6.9.4, 9.6.9.5, 9.6.9.6,
9.6.9.7, 9.6.9.8, 9.6.9.9, 9.6.9.10, 9.6.10.1, 9.6.10.2, 9.6.10.3, 9.6.10.4, 9.6.10.5, 9.6.10.6,
9.6.10.7, 9.6.10.8, 9.6.10.9, 9.6.10.10, 9.7.1.1, 9.7.1.2, 9.7.1.3, 9.7.1.4, 9.7.1.5, 9.7.1.6, 9.7.1.7,
9.7.1.8, 9.7.1.9, 9.7.1.10, 9.7.2.1, 9.7.2.2, 9.7.2.3, 9.7.2.4, 9.7.2.5, 9.7.2.6, 9.7.2.7, 9.7.2.8,
9.7.2.9, 9.7.2.10, 9.7.3.1, 9.7.3.2, 9.7.3.3, 9.7.3.4, 9.7.3.5, 9.7.3.6, 9.7.3.7, 9.7.3.8, 9.7.3.9,
9.7.3.10, 9.7.4.1, 9.7.4.2, 9.7.4.3, 9.7.4.4, 9.7.4.5, 9.7.4.6, 9.7.4.7, 9.7.4.8, 9.7.4.9, 9.7.4.10,
9.7.5.1, 9.7.5.2, 9.7.5.3, 9.7.5.4, 9.7.5.5, 9.7.5.6, 9.7.5.7, 9.7.5.8, 9.7.5.9, 9.7.5.10, 9.7.6.1,
9.7.6.2, 9.7.6.3, 9.7.6.4, 9.7.6.5, 9.7.6.6, 9.7.6.7, 9.7.6.8, 9.7.6.9, 9.7.6.10, 9.7.7.1, 9.7.7.2,
9.7.7.3, 9.7.7.4, 9.7.7.5, 9.7.7.6, 9.7.7.7, 9.7.7.8, 9.7.7.9, 9.7.7.10, 9.7.8.1, 9.7.8.2, 9.7.8.3,
9.7.8.4, 9.7.8.5, 9.7.8.6, 9.7.8.7, 9.7.8.8, 9.7.8.9, 9.7.8.10, 9.7.9.1, 9.7.9.2, 9.7.9.3, 9.7.9.4,
9.7.9.5, 9.7.9.6, 9.7.9.7, 9.7.9.8, 9.7.9.9, 9.7.9.10, 9.7.10.1, 9.7.10.2, 9.7.10.3, 9.7.10.4, 9.7.10.5,
9.7.10.6, 9.7.10.7, 9.7.10.8, 9.7.10.9, 9.7.10.10, 9.8.1.1, 9.8.1.2, 9.8.1.3, 9.8.1.4, 9.8.1.5, 9.8.1.6,
9.8.1.7, 9.8.1.8, 9.8.1.9, 9.8.1.10, 9.8.2.1, 9.8.2.2, 9.8.2.3, 9.8.2.4, 9.8.2.5, 9.8.2.6, 9.8.2.7,
9.8.2.8, 9.8.2.9, 9.8.2.10, 9.8.3.1, 9.8.3.2, 9.8.3.3, 9.8.3.4, 9.8.3.5, 9.8.3.6, 9.8.3.7, 9.8.3.8,
9.8.3.9, 9.8.3.10, 9.8.4.1, 9.8.4.2, 9.8.4.3, 9.8.4.4, 9.8.4.5, 9.8.4.6, 9.8.4.7, 9.8.4.8, 9.8.4.9,
9.8.4.10, 9.8.5.1, 9.8.5.2, 9.8.5.3, 9.8.5.4, 9.8.5.5, 9.8.5.6, 9.8.5.7, 9.8.5.8, 9.8.5.9, 9.8.5.10,
9.8.6.1, 9.8.6.2, 9.8.6.3, 9.8.6.4, 9.8.6.5, 9.8.6.6, 9.8.6.7, 9.8.6.8, 9.8.6.9, 9.8.6.10, 9.8.7.1,
9.8.7.2, 9.8.7.3, 9.8.7.4, 9.8.7.5, 9.8.7.6, 9.8.7.7, 9.8.7.8, 9.8.7.9, 9.8.7.10, 9.8.8.1, 9.8.8.2,
9.8.8.3, 9.8.8.4, 9.8.8.5, 9.8.8.6, 9.8.8.7, 9.8.8.8, 9.8.8.9, 9.8.8.10, 9.8.9.1, 9.8.9.2, 9.8.9.3,
9.8.9.4, 9.8.9.5, 9.8.9.6, 9.8.9.7, 9.8.9.8, 9.8.9.9, 9.8.9.10, 9.8.10.1, 9.8.10.2, 9.8.10.3, 9.8.10.4,
9.8.10.5, 9.8.10.6, 9.8.10.7, 9.8.10.8, 9.8.10.9, 9.8.10.10, 9.9.1.1, 9.9.1.2, 9.9.1.3, 9.9.1.4,
9.9.1.5, 9.9.1.6, 9.9.1.7, 9.9.1.8, 9.9.1.9, 9.9.1.10, 9.9.2.1, 9.9.2.2, 9.9.2.3, 9.9.2.4, 9.9.2.5,
9.9.2.6, 9.9.2.7, 9.9.2.8, 9.9.2.9, 9.9.2.10, 9.9.3.1, 9.9.3.2, 9.9.3.3, 9.9.3.4, 9.9.3.5, 9.9.3.6,
9.9.3.7, 9.9.3.8, 9.9.3.9, 9.9.3.10, 9.9.4.1, 9.9.4.2, 9.9.4.3, 9.9.4.4, 9.9.4.5, 9.9.4.6, 9.9.4.7,
9.9.4.8, 9.9.4.9, 9.9.4.10, 9.9.5.1, 9.9.5.2, 9.9.5.3, 9.9.5.4, 9.9.5.5, 9.9.5.6, 9.9.5.7, 9.9.5.8,
9.9.5.9, 9.9.5.10, 9.9.6.1, 9.9.6.2, 9.9.6.3, 9.9.6.4, 9.9.6.5, 9.9.6.6, 9.9.6.7, 9.9.6.8, 9.9.6.9,
9.9.6.10, 9.9.7.1, 9.9.7.2, 9.9.7.3, 9.9.7.4, 9.9.7.5, 9.9.7.6, 9.9.7.7, 9.9.7.8, 9.9.7.9, 9.9.7.10,
9.9.8.1, 9.9.8.2, 9.9.8.3, 9.9.8.4, 9.9.8.5, 9.9.8.6, 9.9.8.7, 9.9.8.8, 9.9.8.9, 9.9.8.10, 9.9.9.1,
9.9.9.2, 9.9.9.3, 9.9.9.4, 9.9.9.5, 9.9.9.6, 9.9.9.7, 9.9.9.8, 9.9.9.9, 9.9.9.10, 9.9.10.1, 9.9.10.2,
9.9.10.3, 9.9.10.4, 9.9.10.5, 9.9.10.6, 9.9.10.7, 9.9.10.8, 9.9.10.9, 9.9.10.10, 9.10.1.1, 9.10.1.2,
9.10.1.3, 9.10.1.4, 9.10.1.5, 9.10.1.6, 9.10.1.7, 9.10.1.8, 9.10.1.9, 9.10.1.10, 9.10.2.1, 9.10.2.2,
9.10.2.3, 9.10.2.4, 9.10.2.5, 9.10.2.6, 9.10.2.7, 9.10.2.8, 9.10.2.9, 9.10.2.10, 9.10.3.1, 9.10.3.2,
9.10.3.3, 9.10.3.4, 9.10.3.5, 9.10.3.6, 9.10.3.7, 9.10.3.8, 9.10.3.9, 9.10.3.10, 9.10.4.1, 9.10.4.2,
9.10.4.3, 9.10.4.4, 9.10.4.5, 9.10.4.6, 9.10.4.7, 9.10.4.8, 9.10.4.9, 9.10.4.10, 9.10.5.1, 9.10.5.2,
9.10.5.3, 9.10.5.4, 9.10.5.5, 9.10.5.6, 9.10.5.7, 9.10.5.8, 9.10.5.9, 9.10.5.10, 9.10.6.1, 9.10.6.2,
9.10.6.3, 9.10.6.4, 9.10.6.5, 9.10.6.6, 9.10.6.7, 9.10.6.8, 9.10.6.9, 9.10.6.10, 9.10.7.1, 9.10.7.2,
9.10.7.3, 9.10.7.4, 9.10.7.5, 9.10.7.6, 9.10.7.7, 9.10.7.8, 9.10.7.9, 9.10.7.10, 9.10.8.1, 9.10.8.2,
9.10.8.3, 9.10.8.4, 9.10.8.5, 9.10.8.6, 9.10.8.7, 9.10.8.8, 9.10.8.9, 9.10.8.10, 9.10.9.1, 9.10.9.2,
9.10.9.3, 9.10.9.4, 9.10.9.5, 9.10.9.6, 9.10.9.7, 9.10.9.8, 9.10.9.9, 9.10.9.10, 9.10.10.1, 9.10.10.2,
9.10.10.3, 9.10.10.4, 9.10.10.5, 9.10.10.6, 9.10.10.7, 9.10.10.8, 9.10.10.9, 9.10.10.10, 10.1.1.1,
10.1.1.2, 10.1.1.3, 10.1.1.4, 10.1.1.5, 10.1.1.6, 10.1.1.7, 10.1.1.8, 10.1.1.9, 10.1.1.10, 10.1.2.1,
10.1.2.2, 10.1.2.3, 10.1.2.4, 10.1.2.5, 10.1.2.6, 10.1.2.7, 10.1.2.8, 10.1.2.9, 10.1.2.10, 10.1.3.1,
10.1.3.2, 10.1.3.3, 10.1.3.4, 10.1.3.5, 10.1.3.6, 10.1.3.7, 10.1.3.8, 10.1.3.9, 10.1.3.10, 10.1.4.1,
10.1.4.2, 10.1.4.3, 10.1.4.4, 10.1.4.5, 10.1.4.6, 10.1.4.7, 10.1.4.8, 10.1.4.9, 10.1.4.10, 10.1.5.1,
10.1.5.2, 10.1.5.3, 10.1.5.4, 10.1.5.5, 10.1.5.6, 10.1.5.7, 10.1.5.8, 10.1.5.9, 10.1.5.10, 10.1.6.1,
10.1.6.2, 10.1.6.3, 10.1.6.4, 10.1.6.5, 10.1.6.6, 10.1.6.7, 10.1.6.8, 10.1.6.9, 10.1.6.10, 10.1.7.1,
10.1.7.2, 10.1.7.3, 10.1.7.4, 10.1.7.5, 10.1.7.6, 10.1.7.7, 10.1.7.8, 10.1.7.9, 10.1.7.10, 10.1.8.1,
10.1.8.2, 10.1.8.3, 10.1.8.4, 10.1.8.5, 10.1.8.6, 10.1.8.7, 10.1.8.8, 10.1.8.9, 10.1.8.10, 10.1.9.1,
10.1.9.2, 10.1.9.3, 10.1.9.4, 10.1.9.5, 10.1.9.6, 10.1.9.7, 10.1.9.8, 10.1.9.9, 10.1.9.10, 10.1.10.1,
10.1.10.2, 10.1.10.3, 10.1.10.4, 10.1.10.5, 10.1.10.6, 10.1.10.7, 10.1.10.8, 10.1.10.9, 10.1.10.10,
10.2.1.1, 10.2.1.2, 10.2.1.3, 10.2.1.4, 10.2.1.5, 10.2.1.6, 10.2.1.7, 10.2.1.8, 10.2.1.9, 10.2.1.10,
10.2.2.1, 10.2.2.2, 10.2.2.3, 10.2.2.4, 10.2.2.5, 10.2.2.6, 10.2.2.7, 10.2.2.8, 10.2.2.9, 10.2.2.10,
10.2.3.1, 10.2.3.2, 10.2.3.3, 10.2.3.4, 10.2.3.5, 10.2.3.6, 10.2.3.7, 10.2.3.8, 10.2.3.9, 10.2.3.10,
10.2.4.1, 10.2.4.2, 10.2.4.3, 10.2.4.4, 10.2.4.5, 10.2.4.6, 10.2.4.7, 10.2.4.8, 10.2.4.9, 10.2.4.10,
10.2.5.1, 10.2.5.2, 10.2.5.3, 10.2.5.4, 10.2.5.5, 10.2.5.6, 10.2.5.7, 10.2.5.8, 10.2.5.9, 10.2.5.10,
10.2.6.1, 10.2.6.2, 10.2.6.3, 10.2.6.4, 10.2.6.5, 10.2.6.6, 10.2.6.7, 10.2.6.8, 10.2.6.9, 10.2.6.10,
10.2.7.1, 10.2.7.2, 10.2.7.3, 10.2.7.4, 10.2.7.5, 10.2.7.6, 10.2.7.7, 10.2.7.8, 10.2.7.9, 10.2.7.10,
10.2.8.1, 10.2.8.2, 10.2.8.3, 10.2.8.4, 10.2.8.5, 10.2.8.6, 10.2.8.7, 10.2.8.8, 10.2.8.9, 10.2.8.10,
10.2.9.1, 10.2.9.2, 10.2.9.3, 10.2.9.4, 10.2.9.5, 10.2.9.6, 10.2.9.7, 10.2.9.8, 10.2.9.9, 10.2.9.10,
10.2.10.1, 10.2.10.2, 10.2.10.3, 10.2.10.4, 10.2.10.5, 10.2.10.6, 10.2.10.7, 10.2.10.8, 10.2.10.9,
10.2.10.10, 10.3.1.1, 10.3.1.2, 10.3.1.3, 10.3.1.4, 10.3.1.5, 10.3.1.6, 10.3.1.7, 10.3.1.8, 10.3.1.9,
10.3.1.10, 10.3.2.1, 10.3.2.2, 10.3.2.3, 10.3.2.4, 10.3.2.5, 10.3.2.6, 10.3.2.7, 10.3.2.8, 10.3.2.9,
10.3.2.10, 10.3.3.1, 10.3.3.2, 10.3.3.3, 10.3.3.4, 10.3.3.5, 10.3.3.6, 10.3.3.7, 10.3.3.8, 10.3.3.9,
10.3.3.10, 10.3.4.1, 10.3.4.2, 10.3.4.3, 10.3.4.4, 10.3.4.5, 10.3.4.6, 10.3.4.7, 10.3.4.8, 10.3.4.9,
10.3.4.10, 10.3.5.1, 10.3.5.2, 10.3.5.3, 10.3.5.4, 10.3.5.5, 10.3.5.6, 10.3.5.7, 10.3.5.8, 10.3.5.9,
10.3.5.10, 10.3.6.1, 10.3.6.2, 10.3.6.3, 10.3.6.4, 10.3.6.5, 10.3.6.6, 10.3.6.7, 10.3.6.8, 10.3.6.9,
10.3.6.10, 10.3.7.1, 10.3.7.2, 10.3.7.3, 10.3.7.4, 10.3.7.5, 10.3.7.6, 10.3.7.7, 10.3.7.8, 10.3.7.9,
10.3.7.10, 10.3.8.1, 10.3.8.2, 10.3.8.3, 10.3.8.4, 10.3.8.5, 10.3.8.6, 10.3.8.7, 10.3.8.8, 10.3.8.9,
10.3.8.10, 10.3.9.1, 10.3.9.2, 10.3.9.3, 10.3.9.4, 10.3.9.5, 10.3.9.6, 10.3.9.7, 10.3.9.8, 10.3.9.9,
10.3.9.10, 10.3.10.1, 10.3.10.2, 10.3.10.3, 10.3.10.4, 10.3.10.5, 10.3.10.6, 10.3.10.7, 10.3.10.8,
10.3.10.9, 10.3.10.10, 10.4.1.1, 10.4.1.2, 10.4.1.3, 10.4.1.4, 10.4.1.5, 10.4.1.6, 10.4.1.7, 10.4.1.8,
10.4.1.9, 10.4.1.10, 10.4.2.1, 10.4.2.2, 10.4.2.3, 10.4.2.4, 10.4.2.5, 10.4.2.6, 10.4.2.7, 10.4.2.8,
10.4.2.9, 10.4.2.10, 10.4.3.1, 10.4.3.2, 10.4.3.3, 10.4.3.4, 10.4.3.5, 10.4.3.6, 10.4.3.7, 10.4.3.8,
10.4.3.9, 10.4.3.10, 10.4.4.1, 10.4.4.2, 10.4.4.3, 10.4.4.4, 10.4.4.5, 10.4.4.6, 10.4.4.7, 10.4.4.8,
10.4.4.9, 10.4.4.10, 10.4.5.1, 10.4.5.2, 10.4.5.3, 10.4.5.4, 10.4.5.5, 10.4.5.6, 10.4.5.7, 10.4.5.8,
10.4.5.9, 10.4.5.10, 10.4.6.1, 10.4.6.2, 10.4.6.3, 10.4.6.4, 10.4.6.5, 10.4.6.6, 10.4.6.7, 10.4.6.8,
10.4.6.9, 10.4.6.10, 10.4.7.1, 10.4.7.2, 10.4.7.3, 10.4.7.4, 10.4.7.5, 10.4.7.6, 10.4.7.7, 10.4.7.8,
10.4.7.9, 10.4.7.10, 10.4.8.1, 10.4.8.2, 10.4.8.3, 10.4.8.4, 10.4.8.5, 10.4.8.6, 10.4.8.7, 10.4.8.8,
10.4.8.9, 10.4.8.10, 10.4.9.1, 10.4.9.2, 10.4.9.3, 10.4.9.4, 10.4.9.5, 10.4.9.6, 10.4.9.7, 10.4.9.8,
10.4.9.9, 10.4.9.10, 10.4.10.1, 10.4.10.2, 10.4.10.3, 10.4.10.4, 10.4.10.5, 10.4.10.6, 10.4.10.7,
10.4.10.8, 10.4.10.9, 10.4.10.10, 10.5.1.1, 10.5.1.2, 10.5.1.3, 10.5.1.4, 10.5.1.5, 10.5.1.6,
10.5.1.7, 10.5.1.8, 10.5.1.9, 10.5.1.10, 10.5.2.1, 10.5.2.2, 10.5.2.3, 10.5.2.4, 10.5.2.5, 10.5.2.6,
10.5.2.7, 10.5.2.8, 10.5.2.9, 10.5.2.10, 10.5.3.1, 10.5.3.2, 10.5.3.3, 10.5.3.4, 10.5.3.5, 10.5.3.6,
10.5.3.7, 10.5.3.8, 10.5.3.9, 10.5.3.10, 10.5.4.1, 10.5.4.2, 10.5.4.3, 10.5.4.4, 10.5.4.5, 10.5.4.6,
10.5.4.7, 10.5.4.8, 10.5.4.9, 10.5.4.10, 10.5.5.1, 10.5.5.2, 10.5.5.3, 10.5.5.4, 10.5.5.5, 10.5.5.6,
10.5.5.7, 10.5.5.8, 10.5.5.9, 10.5.5.10, 10.5.6.1, 10.5.6.2, 10.5.6.3, 10.5.6.4, 10.5.6.5, 10.5.6.6,
10.5.6.7, 10.5.6.8, 10.5.6.9, 10.5.6.10, 10.5.7.1, 10.5.7.2, 10.5.7.3, 10.5.7.4, 10.5.7.5, 10.5.7.6,
10.5.7.7, 10.5.7.8, 10.5.7.9, 10.5.7.10, 10.5.8.1, 10.5.8.2, 10.5.8.3, 10.5.8.4, 10.5.8.5, 10.5.8.6,
10.5.8.7, 10.5.8.8, 10.5.8.9, 10.5.8.10, 10.5.9.1, 10.5.9.2, 10.5.9.3, 10.5.9.4, 10.5.9.5, 10.5.9.6,
10.5.9.7, 10.5.9.8, 10.5.9.9, 10.5.9.10, 10.5.10.1, 10.5.10.2, 10.5.10.3, 10.5.10.4, 10.5.10.5,
10.5.10.6, 10.5.10.7, 10.5.10.8, 10.5.10.9, 10.5.10.10, 10.6.1.1, 10.6.1.2, 10.6.1.3, 10.6.1.4,
10.6.1.5, 10.6.1.6, 10.6.1.7, 10.6.1.8, 10.6.1.9, 10.6.1.10, 10.6.2.1, 10.6.2.2, 10.6.2.3, 10.6.2.4,
10.6.2.5, 10.6.2.6, 10.6.2.7, 10.6.2.8, 10.6.2.9, 10.6.2.10, 10.6.3.1, 10.6.3.2, 10.6.3.3, 10.6.3.4,
10.6.3.5, 10.6.3.6, 10.6.3.7, 10.6.3.8, 10.6.3.9, 10.6.3.10, 10.6.4.1, 10.6.4.2, 10.6.4.3, 10.6.4.4,
10.6.4.5, 10.6.4.6, 10.6.4.7, 10.6.4.8, 10.6.4.9, 10.6.4.10, 10.6.5.1, 10.6.5.2, 10.6.5.3, 10.6.5.4,
10.6.5.5, 10.6.5.6, 10.6.5.7, 10.6.5.8, 10.6.5.9, 10.6.5.10, 10.6.6.1, 10.6.6.2, 10.6.6.3, 10.6.6.4,
10.6.6.5, 10.6.6.6, 10.6.6.7, 10.6.6.8, 10.6.6.9, 10.6.6.10, 10.6.7.1, 10.6.7.2, 10.6.7.3, 10.6.7.4,
10.6.7.5, 10.6.7.6, 10.6.7.7, 10.6.7.8, 10.6.7.9, 10.6.7.10, 10.6.8.1, 10.6.8.2, 10.6.8.3, 10.6.8.4,
10.6.8.5, 10.6.8.6, 10.6.8.7, 10.6.8.8, 10.6.8.9, 10.6.8.10, 10.6.9.1, 10.6.9.2, 10.6.9.3, 10.6.9.4,
10.6.9.5, 10.6.9.6, 10.6.9.7, 10.6.9.8, 10.6.9.9, 10.6.9.10, 10.6.10.1, 10.6.10.2, 10.6.10.3,
10.6.10.4, 10.6.10.5, 10.6.10.6, 10.6.10.7, 10.6.10.8, 10.6.10.9, 10.6.10.10, 10.7.1.1, 10.7.1.2,
10.7.1.3, 10.7.1.4, 10.7.1.5, 10.7.1.6, 10.7.1.7, 10.7.1.8, 10.7.1.9, 10.7.1.10, 10.7.2.1, 10.7.2.2,
10.7.2.3, 10.7.2.4, 10.7.2.5, 10.7.2.6, 10.7.2.7, 10.7.2.8, 10.7.2.9, 10.7.2.10, 10.7.3.1, 10.7.3.2,
10.7.3.3, 10.7.3.4, 10.7.3.5, 10.7.3.6, 10.7.3.7, 10.7.3.8, 10.7.3.9, 10.7.3.10, 10.7.4.1, 10.7.4.2,
10.7.4.3, 10.7.4.4, 10.7.4.5, 10.7.4.6, 10.7.4.7, 10.7.4.8, 10.7.4.9, 10.7.4.10, 10.7.5.1, 10.7.5.2,
10.7.5.3, 10.7.5.4, 10.7.5.5, 10.7.5.6, 10.7.5.7, 10.7.5.8, 10.7.5.9, 10.7.5.10, 10.7.6.1, 10.7.6.2,
10.7.6.3, 10.7.6.4, 10.7.6.5, 10.7.6.6, 10.7.6.7, 10.7.6.8, 10.7.6.9, 10.7.6.10, 10.7.7.1, 10.7.7.2,
10.7.7.3, 10.7.7.4, 10.7.7.5, 10.7.7.6, 10.7.7.7, 10.7.7.8, 10.7.7.9, 10.7.7.10, 10.7.8.1, 10.7.8.2,
10.7.8.3, 10.7.8.4, 10.7.8.5, 10.7.8.6, 10.7.8.7, 10.7.8.8, 10.7.8.9, 10.7.8.10, 10.7.9.1, 10.7.9.2,
10.7.9.3, 10.7.9.4, 10.7.9.5, 10.7.9.6, 10.7.9.7, 10.7.9.8, 10.7.9.9, 10.7.9.10, 10.7.10.1, 10.7.10.2,
10.7.10.3, 10.7.10.4, 10.7.10.5, 10.7.10.6, 10.7.10.7, 10.7.10.8, 10.7.10.9, 10.7.10.10, 10.8.1.1,
10.8.1.2, 10.8.1.3, 10.8.1.4, 10.8.1.5, 10.8.1.6, 10.8.1.7, 10.8.1.8, 10.8.1.9, 10.8.1.10, 10.8.2.1,
10.8.2.2, 10.8.2.3, 10.8.2.4, 10.8.2.5, 10.8.2.6, 10.8.2.7, 10.8.2.8, 10.8.2.9, 10.8.2.10, 10.8.3.1,
10.8.3.2, 10.8.3.3, 10.8.3.4, 10.8.3.5, 10.8.3.6, 10.8.3.7, 10.8.3.8, 10.8.3.9, 10.8.3.10, 10.8.4.1,
10.8.4.2, 10.8.4.3, 10.8.4.4, 10.8.4.5, 10.8.4.6, 10.8.4.7, 10.8.4.8, 10.8.4.9, 10.8.4.10, 10.8.5.1,
10.8.5.2, 10.8.5.3, 10.8.5.4, 10.8.5.5, 10.8.5.6, 10.8.5.7, 10.8.5.8, 10.8.5.9, 10.8.5.10, 10.8.6.1,
10.8.6.2, 10.8.6.3, 10.8.6.4, 10.8.6.5, 10.8.6.6, 10.8.6.7, 10.8.6.8, 10.8.6.9, 10.8.6.10, 10.8.7.1,
10.8.7.2, 10.8.7.3, 10.8.7.4, 10.8.7.5, 10.8.7.6, 10.8.7.7, 10.8.7.8, 10.8.7.9, 10.8.7.10, 10.8.8.1,
10.8.8.2, 10.8.8.3, 10.8.8.4, 10.8.8.5, 10.8.8.6, 10.8.8.7, 10.8.8.8, 10.8.8.9, 10.8.8.10, 10.8.9.1,
10.8.9.2, 10.8.9.3, 10.8.9.4, 10.8.9.5, 10.8.9.6, 10.8.9.7, 10.8.9.8, 10.8.9.9, 10.8.9.10, 10.8.10.1,
10.8.10.2, 10.8.10.3, 10.8.10.4, 10.8.10.5, 10.8.10.6, 10.8.10.7, 10.8.10.8, 10.8.10.9, 10.8.10.10,
10.9.1.1, 10.9.1.2, 10.9.1.3, 10.9.1.4, 10.9.1.5, 10.9.1.6, 10.9.1.7, 10.9.1.8, 10.9.1.9, 10.9.1.10,
10.9.2.1, 10.9.2.2, 10.9.2.3, 10.9.2.4, 10.9.2.5, 10.9.2.6, 10.9.2.7, 10.9.2.8, 10.9.2.9, 10.9.2.10,
10.9.3.1, 10.9.3.2, 10.9.3.3, 10.9.3.4, 10.9.3.5, 10.9.3.6, 10.9.3.7, 10.9.3.8, 10.9.3.9, 10.9.3.10,
10.9.4.1, 10.9.4.2, 10.9.4.3, 10.9.4.4, 10.9.4.5, 10.9.4.6, 10.9.4.7, 10.9.4.8, 10.9.4.9, 10.9.4.10,
10.9.5.1, 10.9.5.2, 10.9.5.3, 10.9.5.4, 10.9.5.5, 10.9.5.6, 10.9.5.7, 10.9.5.8, 10.9.5.9, 10.9.5.10,
10.9.6.1, 10.9.6.2, 10.9.6.3, 10.9.6.4, 10.9.6.5, 10.9.6.6, 10.9.6.7, 10.9.6.8, 10.9.6.9, 10.9.6.10,
10.9.7.1, 10.9.7.2, 10.9.7.3, 10.9.7.4, 10.9.7.5, 10.9.7.6, 10.9.7.7, 10.9.7.8, 10.9.7.9, 10.9.7.10,
10.9.8.1, 10.9.8.2, 10.9.8.3, 10.9.8.4, 10.9.8.5, 10.9.8.6, 10.9.8.7, 10.9.8.8, 10.9.8.9, 10.9.8.10,
10.9.9.1, 10.9.9.2, 10.9.9.3, 10.9.9.4, 10.9.9.5, 10.9.9.6, 10.9.9.7, 10.9.9.8, 10.9.9.9, 10.9.9.10,
10.9.10.1, 10.9.10.2, 10.9.10.3, 10.9.10.4, 10.9.10.5, 10.9.10.6, 10.9.10.7, 10.9.10.8, 10.9.10.9,
10.9.10.10, 10.10.1.1, 10.10.1.2, 10.10.1.3, 10.10.1.4, 10.10.1.5, 10.10.1.6, 10.10.1.7, 10.10.1.8,
10.10.1.9, 10.10.1.10, 10.10.2.1, 10.10.2.2, 10.10.2.3, 10.10.2.4, 10.10.2.5, 10.10.2.6, 10.10.2.7,
10.10.2.8, 10.10.2.9, 10.10.2.10, 10.10.3.1, 10.10.3.2, 10.10.3.3, 10.10.3.4, 10.10.3.5, 10.10.3.6,
10.10.3.7, 10.10.3.8, 10.10.3.9, 10.10.3.10, 10.10.4.1, 10.10.4.2, 10.10.4.3, 10.10.4.4, 10.10.4.5,
10.10.4.6, 10.10.4.7, 10.10.4.8, 10.10.4.9, 10.10.4.10, 10.10.5.1, 10.10.5.2, 10.10.5.3, 10.10.5.4,
10.10.5.5, 10.10.5.6, 10.10.5.7, 10.10.5.8, 10.10.5.9, 10.10.5.10, 10.10.6.1, 10.10.6.2, 10.10.6.3,
10.10.6.4, 10.10.6.5, 10.10.6.6, 10.10.6.7, 10.10.6.8, 10.10.6.9, 10.10.6.10, 10.10.7.1, 10.10.7.2,
10.10.7.3, 10.10.7.4, 10.10.7.5, 10.10.7.6, 10.10.7.7, 10.10.7.8, 10.10.7.9, 10.10.7.10, 10.10.8.1,
10.10.8.2, 10.10.8.3, 10.10.8.4, 10.10.8.5, 10.10.8.6, 10.10.8.7, 10.10.8.8, 10.10.8.9, 10.10.8.10,
10.10.9.1, 10.10.9.2, 10.10.9.3, 10.10.9.4, 10.10.9.5, 10.10.9.6, 10.10.9.7, 10.10.9.8, 10.10.9.9,
10.10.9.10, 10.10.10.1, 10.10.10.2, 10.10.10.3, 10.10.10.4, 10.10.10.5, 10.10.10.6, 10.10.10.7,
10.10.10.8, 10.10.10.9, 10.10.10.10

Additional exemplary compound groups include the following compound groups disclosed below. Unless otherwise specified, the configurations of all hydrogen atoms and R groups for the following compound groups are as defined for the group 1 compounds above. As is apparent from the description, each of the compound groups disclose a number of unique compounds or generic structures. The compounds or generic structures specifically described in any of the compound groups are thus exemplary only and the remaining compounds or structures in each group are described by Tables A and B.

As used in the description of compounds in the compound groups, the definitive structure of compounds in the various compound groups is specified only by the structure defining portion of the compound group and in Tables A and B, which together definitively name or specify individual compound or genus structures. The structure-defining portion of the compound groups is generally contained in the first sentence of the compound groups below and in the following paragraph. This applies regardless of any name or structure, including chemical names in the exemplary compounds that are named in some of the compound groups. Thus, any name or structure for any compound or compound genus that refers to a compound or genus in a compound group and is given anywhere in the disclosure is intended only to refer to the compound or genus that is definitively specified by the compound groups together with Tables A and B.

For the following compound groups, reference to an androstene or a 5α-androstene with no double bond at the 4-5 or 5-6 position means that the hydrogen atom or other moiety at the 5-position is in the α-configuration. For androstenes with no double bond at the 4-5 or 5-6 position and a hydrogen atom or other moiety at the 5-position in the β-configuration will usually be referred to as a 5β-androstene. For compound groups where a double bond is present at the 1-2 or 2-3 position and/or when R⁹ is substituted, R⁹ will be ═CH—, ═CR¹⁰—, —CHR¹⁰—, —C(R¹⁰)₂— or another moiety defined for R⁹ herein, instead of —CH₂—. For compound groups where a double bond is present at the 9-11 position and/or when R⁸ is substituted, R⁸ will be ═CH—, ═CR¹⁰—, —CHR¹⁰—, —C(R¹⁰)₂— or another moiety defined for R⁸ herein, instead of —CH₂—. 9-11 and/or 15-16 positions. For compound groups where a double bond is present at the 15-16 position and/or when R⁷is substituted, R⁷ will be ═CH—, ═CR¹⁰—, —CHR¹⁰—, —C(R¹⁰)₂— or another moiety defined for R⁷ herein, instead of —CH₂—.

Group 2. This group comprises compounds named in Table B having R¹, R², R³ and R⁴ substituents defined in Table A wherein the R¹, R², R³ and R⁴ substituents are bonded to the steroid nucleus described for group 1 compounds, except that R^{10E} is hydrogen in the β-configuration. Exemplary group 2 compounds include 1.2.4.1, which is 3,7β-dihydroxy-16α-fluoro-17β-amino-5β-androst-1,3-diene, 1.1.5.9, which is 3,17β-dihydroxy-5β-androst-1,3-diene, 1.1.7.1, which is 3-hydroxy-16α-acetoxy-17β-amino-5β-androst-1,3-diene and compound 1.1.4.10, which is 3-hydroxy-16α-fluoro-17β-acetoxy-5β-androst-1,3-diene. Other exemplary group 2 compounds include 3,17β-dihydroxy-7α-methyl-5β-androst-1,3-diene, 3,17β-dihydroxy-7β-ethynyl-5β-androst-1,3-diene, 3,17β-dihydroxy-7β-methoxy-5β-androst-1,3-diene, 3,7β,17β-trihydroxy-5β-androst-1,3-diene, 3-amino-17β-hydroxy-5β-androst-1,3-diene, 3-amino-7β,17β-dihydroxy-5β-androst-1,3-diene, 3-hydroxy-17β-amino-5β-androst-1,3-diene, 3,7β-dihydroxy-17β-amino-5β-androst-1,3-diene, 3,17β-dihydroxy-7β-amino-5β-androst-1,3-diene, 3-hydroxy-7β,17β-diacetylamino-5β-androst-1,3-diene, 3-hydroxy-7β,17β-dimethylamino-5β-androst-1,3-diene and 16α-hydroxy, 16β-hydroxy, 16α-methyl, 16α-amino, 16α-aminomethyl, 16α-acetate and 16α-halo analogs of any group 2 compound.

Group 3. This group comprises compounds named in Table B having R¹, R², R³ and R⁴ substituents defined in Table A wherein the R¹, R², R³ and R⁴ substituents are bonded to the steroid nucleus described for group 1 compounds, except that R^{10E} is absent and double bonds are present at the 1-2, 3-4 and 5-6 positions. Exemplary group 3 compounds include 1.2.4.1, which is 3,7β-dihydroxy-16α-fluoro-17β-aminoandrost-1,3,5-triene, 1.1.5.9, which is 3,17β-dihydroxyandrost-1,3,5-triene, 1.1.7.1, which is 3-hydroxy-16α-acetoxy-17β-aminoandrost-1,3,5-triene and compound 1.1.4.10, which is 3-hydroxy-16α-fluoro-17β-acetoxyandrost-1,3,5-triene. Other exemplary group 3 compounds include 3,17β-dihydroxy-7β-acetoxyandrost-1,3,5-triene, 3,17β-dihydroxy-17α-methylandrost-1,3,5-triene, 3,17β-dihydroxy-17α-ethynylandrost-1,3,5-triene, and 16α-hydroxy, 16-oxo, 16β-hydroxy, 16α-methyl, 16α-amino, 16α-aminomethyl, 16α-acetate and 16α-halo analogs of any group 3 compound.

Group 4. This group comprises compounds named in Table B having R¹, R², R³ and R⁴ substituents defined in Table A wherein the R¹, R², R³ and R⁴ substituents are bonded to the steroid nucleus described for group 1 compounds, except that double bonds are present at the 1-2, 3-4 and 16-17 positions. Exemplary group 4 compounds include 1.2.4.1, which is 3,7β-dihydroxy-16-fluoro-17-aminoandrost-1,3,16-triene, 1.1.5.9, which is 3,17-dihydroxyandrost-1,3,16-triene, 1.1.7.1, which is 3-hydroxy-16-acetoxy-17-aminoandrost-1,3,16-triene and compound 1.1.4.10, which is 3-hydroxy-16-fluoro-17-acetoxyandrost-1,3,16-triene. Other exemplary group 4 compounds include 3,17-dihydroxy-7β-acetoxyandrost-1,3,16-triene, 3,17-dihydroxy-7β-methylandrost-1,3,16-triene, 3,17-dihydroxy-7β-methoxyandrost-1,3,16-triene, 3,7β,17-trihydroxyandrost-1,3,16-triene, 3-amino-17-hydroxyandrost-1,3,16-triene, 3-amino-7β,17-dihydroxyandrost-1,3,16-triene, 3-hydroxy-17-aminoandrost-1,3,16-triene, 3,7β-dihydroxy-17-aminoandrost-1,3,16-triene, 3,17-dihydroxy-7β-aminoandrost-1,3,16-triene, 3-hydroxy-7β,17-diacetylaminoandrost-1,3,16-triene, 3-hydroxy-7β,17-dimethylaminoandrost-1,3,16-triene and 16-hydroxy, 16-methyl, 16-amino, 16-aminomethyl, 16-acetate and 16-halo analogs of any of these compounds.

Group 5. This group comprises compounds named in Table B having R¹, R², R³ and R⁴ substituents defined in Table A wherein the R¹, R², R³ and R⁴ substituents are bonded to the steroid nucleus described for group 1 compounds, except that R^{10E} is present in the β-configuration and double bonds are present at the 1-2, 3-4 and 16-17 positions. Exemplary group 5 compounds include 1.2.4.1, which is 3,7β-dihydroxy-16-fluoro-17-amino-5β-androst-1,3,16-triene, 1.1.5.9, which is 3,17-dihydroxy-5β-androst-1,3,16-triene, 1.1.7.1, which is 3-hydroxy-16-acetoxy-17-amino-5β-androst-1,3,16-triene and compound 1.1.4.10, which is 3-hydroxy-16-fluoro-17-acetoxy-5β-androst-1,3,16-triene. Other exemplary group 5 compounds include 3,17-dihydroxy-7β-acetoxy-5β-androst-1,3,16-triene, 3,17-dihydroxy-7β-methyl-5β-androst-1,3,16-triene, 3,17-dihydroxymethoxy-5β-androst-1,3,16-triene, 3,7β,17-trihydroxy-5β-androst-1,3,16-triene, 3-amino-17-hydroxy-5β-androst-1,3,16-triene, 3-amino-7β,17-dihydroxy-5β-androst-1,3,16-triene, 3-hydroxy-17-amino-5β-androst-1,3,16-triene, 3,7β-dihydroxy-17-amino-5β-androst-1,3,16-triene, 3,17-dihydroxy-7β-amino-5β-androst-1,3,16-triene, 3-hydroxy-7β,17-diacetylamino-5β-androst-1,3,16-triene, 3-hydroxy-7β,17-dimethylamino-5β-androst-1,3,16-triene and 16-hydroxy, 16-methyl, 16-amino, 16-aminomethyl, 16-acetate and 16-halo analogs of any of these compounds.

Group 6. This group comprises compounds named in Table B having R¹, R², R³ and R⁴ substituents defined in Table A wherein the R¹, R², R³ and R⁴ substituents are bonded to the steroid nucleus described for group 1 compounds, except that R¹is in the β-configuration, R^{10E} is absent and double bonds are present at the 1-2 and 5-6 positions. Exemplary group 6 compounds include 1.2.4.1, which is 3β,7β-dihydroxy-16α-fluoro-17β-aminoandrost-1,5-diene, 1.1.5.9, which is 3β,17β-dihydroxyandrost-1,5-diene, 1.1.7.1, which is 3β-hydroxy-16α-acetoxy-17β-aminoandrost-1,5-diene and compound 1.1.4.10, which is 3β-hydroxy-16α-fluoro-17β-acetoxyandrost-1,5-diene. Other exemplary group 6 compounds include 3β,17β-dihydroxy-7β-acetoxyandrost-1,5-diene, 3β,17β-dihydroxy-7β-methylandrost-1,5-diene, 3β,17β-dihydroxy-7β-methoxyandrost-1,5-diene, 3β,7β,17β-trihydroxyandrost-1,5-diene, 3β-amino-17β-hydroxyandrost-1,5-diene, 3β-amino-7β,17β-dihydroxyandrost-1,5-diene, 3β-hydroxy-17β-aminoandrost-1,5-diene, 3β,7β-dihydroxy-17β-aminoandrost-1,5-diene, 3β,17β-dihydroxy-7β-aminoandrost-1,5-diene, 3β-hydroxy-7β,17β-diacetylaminoandrost-1,5-diene, 3β-hydroxy-7β,17β-dimethylaminoandrost-1,5-diene and 16α-hydroxy, 16α-methyl, 16α-amino, 16α-aminomethyl, 16α-acetate and 16α-halo analogs of any of these compounds.

Group 7. This group comprises compounds named in Table B having R¹, R², R³ and R⁴ substituents defined in Table A wherein the R¹, R², R³ and R⁴ substituents are bonded to the steroid nucleus described for group 1 compounds, except that R¹ is in the β-configuration and double bonds are present at the 1-2 and 6-7 positions. Exemplary group 7 compounds include 1.2.4.1, which is 3β,7β-dihydroxy-16α-fluoro-17β-aminoandrost-1,6-diene, 1.1.5.9, which is 3β,17β-dihydroxyandrost-1,6-diene, 1.1.7.1, which is 3β-hydroxy-16α-acetoxy-17β-aminoandrost-1,6-diene and compound 1.1.4.10, which is 3β-hydroxy-16α-fluoro-17β-acetoxyandrost-1,6-diene. Other exemplary group 7 compounds include 3β,17β-dihydroxy-7-acetoxyandrost-1,6-diene, 3β,17β-dihydroxy-7-methylandrost-1,6-diene, 3β,17β-dihydroxy-7-methoxyandrost-1,6-diene, 3β,7,17β-trihydroxyandrost-1,6-diene, 3β-amino-17β-hydroxyandrost-1,6-diene, 3β-amino-7,17β-dihydroxyandrost-1,6-diene, 3β-hydroxy-17β-aminoandrost-1,6-diene, 3β,7-dihydroxy-17β-aminoandrost-1,6-diene, 3β,17β-dihydroxy-7β-aminoandrost-1,6-diene, 3β-hydroxy-7,17β-diacetylaminoandrost-1,6-diene, 3β-hydroxy-7,17β-dimethylaminoandrost-1,6-diene and 16α-hydroxy, 16α-methyl, 16α-amino, 16α-aminomethyl, 16α-acetate and 16α-halo analogs of any of these compounds.

Group 8. This group comprises compounds named in Table B having R¹, R², R³ and R⁴ substituents defined in Table A wherein the R¹, R², R³ and R⁴ substituents are bonded to the steroid nucleus described for group 1 compounds, except that R¹ is in the β-configuration, R^{10E} is in the β-configuration and double bonds are present at the 1-2 and 6-7 positions. Exemplary group 8 compounds include 1.2.4.1, which is 3β,7β-dihydroxy-16α-fluoro-17β-amino-5β-androst-1,6-diene, 1.1.5.9, which is 3β,17β-dihydroxy-5β-androst-1,6-diene, 1.1.7.1, which is 3β-hydroxy-16α-acetoxy-17β-amino-5β-androst-1,6-diene and compound 1.1.4.10, which is 3β-hydroxy-16α-fluoro-17β-acetoxy-5β-androst-1,6-diene. Other exemplary group 8 compounds include 3β,17β-dihydroxy-7-acetoxy-5β-androst-1,6-diene, 3β,17β-dihydroxy-7-methyl-5β-androst-1,6-diene, 3β,17β-dihydroxy-7-methoxy-5β-androst-1,6-diene, 3β,7,17β-trihydroxy-5β-androst-1,6-diene, 3β-amino-17β-hydroxy-5β-androst-1,6-diene, 3β-amino-7,17β-dihydroxy-5β-androst-1,6-diene, 3β-hydroxy-17β-amino-5β-androst-1,6-diene, 3β,7-dihydroxy-17β-amino-5β-androst-1,6-diene, 3β,17β-dihydroxy-7β-amino-5β-androst-1,6-diene, 3β-hydroxy-7,17β-diacetylamino-5β-androst-1,6-diene, 3β-hydroxy-7,17β-dimethylamino-5β-androst-1,6-diene and 16α-hydroxy, 16α-methyl, 16α-amino, 16α-aminomethyl, 16α-acetate and 16α-halo analogs of any of these compounds.

Group 9. This group comprises compounds named in Table B having R¹, R², R³ and R⁴ substituents defined in Table A wherein the R¹, R², R³ and R⁴ substituents are bonded to the steroid nucleus described for group 1 compounds, except that R¹ is in the β-configuration, R^{10F} is absent and double bonds are present at the 1-2 and 7-8 positions. Exemplary group 9 compounds include 1.2.4.1, which is 3β,7β-dihydroxy-16α-fluoro-17β-aminoandrost-1,7-diene, 1.1.5.9, which is 3β,17β-dihydroxyandrost-1,7-diene, 1.1.7.1, which is 3β-hydroxy-16α-acetoxy-17β-aminoandrost-1,7-diene and compound 1.1.4.10, which is 3β-hydroxy-16α-fluoro-17β-acetoxyandrost-1,7-diene. Other exemplary group 9 compounds include 3β,17β-dihydroxy-7-acetoxyandrost-1,7-diene, 3β,17β-dihydroxy-7-methylandrost-1,7-diene, 3β,17β-dihydroxy-7-methoxyandrost-1,7-diene, 3β,7,17β-trihydroxyandrost-1,7-diene, 3β-amino-17β-hydroxyandrost-1,7-diene, 3β-amino-7,17β-dihydroxyandrost-1,7-diene, 3β-hydroxy-17β-aminoandrost-1,7-diene, 3β,7-dihydroxy-17β-aminoandrost-1,7-diene, 3β,17-dihydroxy-7β-aminoandrost-1,7-diene, 3β-hydroxy-7,17β-diacetylaminoandrost-1,7-diene, 3β-hydroxy-7,17β-dimethylaminoandrost-1,7-diene and 16α-hydroxy, 16α-methyl, 16α-amino, 16α-aminomethyl, 16α-acetate and 16α-halo analogs of any of these compounds.

Group 10. This group comprises compounds named in Table B having R¹, R², R³ and R⁴ substituents defined in Table A wherein the R¹, R², R³ and R⁴ substituents are bonded to the steroid nucleus described for group 1 compounds, except that R¹ is in the β-configuration, R^{10E} is in the β-configuration, R^{10F} is absent and double bonds are present at the 1-2 and 7-8 positions. Exemplary group 10 compounds include 1.2.4.1, which is 3β,7-dihydroxy-16α-fluoro-17β-amino-5β-androst-1,7-diene, 1.1.5.9, which is 3β,17β-dihydroxy-5β-androst-1,7-diene, 1.1.7.1, which is 3β-hydroxy-16α-acetoxy-17β-amino-5β-androst-1,7-diene and compound 1.1.4.10, which is 3β-hydroxy-16α-fluoro-17β-acetoxy-5β-androst-1,7-diene. Other exemplary group 10 compounds include 3β,17β-dihydroxy-7-acetoxy-5β-androst-1,7-diene, 3β,17β-dihydroxy-7-methyl-5β-androst-1,7-diene, 3β,17β-dihydroxy-7-methoxy-5β-androst-1,7-diene, 3β,7β,17β-trihydroxy-5β-androst-1,7-diene, 3β-amino-17β-hydroxy-5β-androst-1,7-diene, 3β-amino-7,17β-dihydroxy-5β-androst-1,7-diene, 3β-hydroxy-17β-amino-5β-androst-1,7-diene, 3β,7-dihydroxy-17β-amino-5β-androst-1,7-diene, 3β,17-dihydroxy-7β-amino-5β-androst-1,7-diene, 3β-hydroxy-7,17β-diacetylamino-5β-androst-1,7-diene, 3β-hydroxy-7,17β-dimethylamino-5β-androst-1,7-diene and 16α-hydroxy, 16α-methyl, 16α-amino, 16α-aminomethyl, 16α-acetate and 16α-halo analogs of any of these compounds.

Group 11. This group comprises compounds named in Table B having R¹, R R³ and R⁴ substituents defined in Table A wherein the R¹, R², R³ and R⁴ substituents are bonded to the steroid nucleus described for group 1 compounds, except that R¹ is in the β-configuration, R^{10F} and R^{10G} are absent and double bonds are present at the 1-2 and 8-9 positions. Exemplary group 11 compounds include 1.2.4. 1, which is 3β,7β-dihydroxy-16α-fluoro-17β-aminoandrost-1,8(9)-diene, 1.1.5.9, which is 3β,17β-dihydroxyandrost-1,8(9)-diene, 1.1.7.1, which is 3β-hydroxy-16α-acetoxy-17β-aminoandrost-1,8(9)-diene and compound 1.1.4.10, which is 3β-hydroxy-16α-fluoro-17β-acetoxyandrost-1,8(9)-diene. Other exemplary group 11 compounds include 3β,17β-dihydroxy-7β-acetoxyandrost-1,8(9)-diene, 3β,17β-dihydroxy-7β-methylandrost-1,8(9)-diene, 3β,17β-dihydroxy-7β-methoxyandrost-1,8(9)-diene, 3β,7β,17β-trihydroxyandrost-1,8(9)-diene, 3β-amino-17β-hydroxyandrost-1,8(9)-diene, 3β-amino-7β,17β-dihydroxyandrost-1,8(9)-diene, 3β-hydroxy-17β-aminoandrost-1,8(9)-diene, 3β,7β-dihydroxy-17β-aminoandrost-1,8(9)-diene, 3β,17β-dihydroxy-7β-aminoandrost-1,8(9)-diene, 3β-hydroxy-7β,17β-diacetylaminoandrost-1,8(9)-diene, 3β-hydroxy-7β,17β-dimethylaminoandrost-1,8(9)-diene and 16α-hydroxy, 16α-methyl, 16α-amino, 16α-aminomethyl, 16α-acetate and 16α-halo analogs of any of these compounds.

Group 12. This group comprises compounds named in Table B having R¹, R², R³ and R⁴ substituents defined in Table A wherein the R¹, R², R³ and R⁴ substituents are bonded to the steroid nucleus described for group 1 compounds, except that R¹ is in the β-configuration, R^{10E} is in the β-configuration, R^{10F} and R^{10G} are absent and double bonds are present at the 1-2 and 8-9 positions. Exemplary group 12 compounds include 1.2.4.1, which is 3β,7β-dihydroxy-16α-fluoro-17β-amino-5β-androst-1,8(9)-diene, 1.1.5.9, which is 3β,17β-dihydroxy-5β-androst-1,8(9)-diene, 1.1.7.1, which is 3β-hydroxy-16α-acetoxy-17β-amino-5β-androst-1,8(9)-diene and compound 1.1.4.10, which is 3β-hydroxy-16α-fluoro-17β-acetoxy-5β-androst-1,8(9)-diene. Other exemplary group 12 compounds include 3β,17β-dihydroxy-7β-acetoxy-5β-androst-1,8(9)-diene, 3β,17β-dihydroxy-7β-methyl-5β-androst-1,8(9)-diene, 3β,17β-dihydroxy-7β-methoxy-5β-androst-1,8(9)-diene, 3β,7β,17β-trihydroxy-5β-androst-1,8(9)-diene, 3β-amino-17β-hydroxy-5β-androst-1,8(9)-diene, 3β-amino-7β,17β-dihydroxy-5β-androst-1,8(9)-diene, 3β-hydroxy-17β-amino-5β-androst-1,8(9)-diene, 3β,7β-dihydroxy-17β-amino-5β-androst-1,8(9)-diene, 3β,17β-dihydroxy-7β-amino-5β-androst-1,8(9)-diene, 3β-hydroxy-7β,17β-diacetylamino-5β-androst-1,8(9)-diene, 3β-hydroxy-7β,17β-dimethylamino-5β-androst-1,8(9)-diene and 16α-hydroxy, 16α-methyl, 16α-amino, 16α-aminomethyl, 16α-acetate and 16α-halo analogs of any of these compounds.

Group 13. This group comprises compounds named in Table B having R¹, R², R³ and R⁴ substituents defined in Table A wherein the R¹, R², R³ and R⁴ substituents are bonded to the steroid nucleus described for group 1 compounds, except that R¹ is in the β-configuration, R^{10F} and R^{10H} are absent and double bonds are present at the 1-2 and 8-14 positions. Exemplary group 13 compounds include 1.2.4.1, which is 3β,7β-dihydroxy-16α-fluoro-17β-aminoandrost-1,8(14)-diene, 1.1.5.9, which is 3β,17β-dihydroxyandrost-1,8(14)-diene, 1.1.7.1, which is 3β-hydroxy-16α-acetoxy-17β-aminoandrost-1,8(14)-diene and compound 1.1.4.10, which is 3β-hydroxy-16α-fluoro-17β-acetoxyandrost-1,8(14)-diene. Other exemplary group 13 compounds include 3β,17β-dihydroxy-7β-acetoxyandrost-1,8(14)-diene, 3β,17β-dihydroxy-7β-methylandrost-1,8(14)-diene, 3β,17β-dihydroxy-7β-methoxyandrost-1,8(14)-diene, 3β,7β,17β-trihydroxyandrost-1,8(14)-diene, 3β-amino-17β-hydroxyandrost-1,8(14)-diene, 3β-amino-7β,17β-dihydroxyandrost-1,8(14)-diene, 3β-hydroxy-17β-aminoandrost-1,8(14)-diene, 3β,7β-dihydroxy-17β-aminoandrost-1,8(14)-diene, 3β,17β-dihydroxy-7β-aminoandrost-1,8(14)-diene, 3β-hydroxy-7β,17β-diacetylaminoandrost-1,8(14)-diene, 3β-hydroxy-7β,17β-dimethylaminoandrost-1,8(14)-diene and 16α-hydroxy, 16α-methyl, 16α-amino, 16α-aminomethyl, 16α-acetate and 16α-halo analogs of any of these compounds.

Group 14. This group comprises compounds named in Table B having R¹, R², R³ and R⁴ substituents defined in Table A wherein the R¹, R², R³ and R⁴ substituents are bonded to the steroid nucleus described for group 1 compounds, except that R¹ is in the β-configuration, R^{10E} is in the β-configuration, R^{10F} and R^{10H} are absent and double bonds are present at the 1-2 and 8-9 positions. Exemplary group 14 compounds include 1.2.4.1, which is 3β,7β-dihydroxy-16α-fluoro-17β-amino-5β-androst-1,8(14)-diene, 1.1.5.9, which is 3β,17β-dihydroxy-5β-androst-1,8(14)-diene, 1.1.7.1, which is 3β-hydroxy-16α-acetoxy-17β-amino-5β-androst-1,8(14)-diene and compound 1.1.4.10, which is 3β-hydroxy-16α-fluoro-17β-acetoxy-5β-androst-1,8(14)-diene. Other exemplary group 14 compounds include 3β,17β-dihydroxy-7β-acetoxy-5β-androst-1,8(14)-diene, 3β,17β-dihydroxy-7β-methyl-5β-androst-1,8(14)-diene, 3β,17β-dihydroxy-7β-methoxy-5β-androst-1,8(14)-diene, 3β,7β,17β-trihydroxy-5β-androst-1,8(14)-diene, 3β-amino-17β-hydroxy-5β-androst-1,8(14)-diene, 3β-amino-7β,17β-dihydroxy-5β-androst-1,8(14)-diene, 3β-hydroxy-17β-amino-5β-androst-1,8(14)-diene, 3β,7β-dihydroxy-17β-amino-5β-androst-1,8(14)-diene, 3β,17β-dihydroxy-7β-amino-5β-androst-1,8(14)-diene, 3β,17β-dihydroxy-7β-aminoandrost-1,8(14)-diene, 3β-hydroxy-7β,17β-diacetylamino-5β-androst-1,8(14)-diene, 3β-hydroxy-7β,17β-dimethylamino-5β-androst-1,8(14)-diene and 16α-hydroxy, 16α-methyl, 16α-amino, 16α-aminomethyl, 16α-acetate and 16α-halo analogs of any of these compounds.

Group 15. This group comprises compounds named in Table B having R¹, R², R³ and R⁴ substituents defined in Table A wherein the R¹, R², R³ and R⁴ substituents are bonded to the steroid nucleus described for group 1 compounds, except that R¹ is in the β-configuration and double bonds are present at the 1-2 and 15-16 positions. Exemplary group 15 compound 1.2.4.1 is 3β,7β-dihydroxy-16-fluoro-17β-aminoandrost-1,15-diene, compound 1.1.5.9 is 3β,17β-dihydroxyandrost-1,15-diene, 1.1.7.1, which is 3β-hydroxy-16-acetoxy-17β-aminoandrost-1,15-diene and compound 1.1.4.10, which is 3β-hydroxy-16-fluoro-17β-acetoxyandrost-1,15-diene. Other exemplary group 15 compounds include 3,17β-dihydroxy-7β-acetoxyandrost-1,15-diene, 3β,17β-dihydroxy-7β-methylandrost-1,15-diene, 3β,17β-dihydroxy-7β-methoxyandrost-1,15-diene, 3β,7β,17β-trihydroxyandrost-1,15-diene, 3β-amino-17β-hydroxyandrost-1,15-diene, 3β-amino-7β,17β-dihydroxyandrost-1,15-diene, 3β-hydroxy-17β-aminoandrost-1,15-diene, 3β,7β-dihydroxy-17β-aminoandrost-1,15-diene, 3β,17β-dihydroxy-7β-aminoandrost-1,15-diene, 3,17β-dihydroxy-7β-aminoandrost-1,15-diene, 3β-hydroxy-7β,17β-diacetylaminoandrost-1,15-diene, 3β-hydroxy-7β,17β-dimethylaminoandrost-1,15-diene and 16-hydroxy, 16-methyl, 16-amino, 16-aminomethyl, 16-acetate and 16-halo analogs of any of these compounds.

Group 16. This group comprises compounds named in Table B having R¹, R², R³ and R⁴ substituents defined in Table A wherein the R¹, R², R³ and R⁴ substituents are bonded to the steroid nucleus described for group 1 compounds, except that R¹ is in the β-configuration, R^{10E} is in the β-configuration and double bonds are present at the 1-2 and 15-16 positions. Exemplary group 16 compound 1.2.4.1 is 3⊖,7β-dihydroxy-16-fluoro-17β-amino-5β-androst-1,15-diene, compound 1.1.5.9 is 3β,17β-dihydroxy-5β-androst-1,15-diene, 1.1.7.1, which is 3β-hydroxy-16-acetoxy-17β-amino-5β-androst-1,15-diene and compound 1.1.4.10, which is 3β-hydroxy-16-fluoro-17β-acetoxy-5β-androst-1,15-diene. Other exemplary group 16 compounds include 3β,17β-dihydroxy-7β-acetoxy-5β-androst-1,15-diene, 3β,17β-dihydroxy-7β-methyl-5β-androst-1,15-diene, 3β,17β-dihydroxy-7β-methoxy-5β-androst-1,15-diene, 3β,7β,17β-trihydroxy-5β-androst-1,15-diene, 3β-amino-17β-hydroxy-5β-androst-1,15-diene, 3β-amino-7β,17β-dihydroxy-5β-androst-1,15-diene, 3β-hydroxy-17β-amino-5β-androst-1,15-diene, 3β,7β-dihydroxy-17β-amino-5β-androst-1,15-diene, 3β,17β-dihydroxy-7β-amino-5β-androst-1,15-diene, 3β-hydroxy-7β,17β-diacetylamino-5β-androst-1,15-diene, 3β-hydroxy-7β,17β-dimethylamino-5β-androst-1,15-diene and 16-hydroxy, 16-methyl, 16-amino, 16-aminomethyl, 16-acetate and 16-halo analogs of any of these compounds.

Group 17. This group comprises compounds named in Table B having R¹, R², R³ and R⁴ substituents defined in Table A wherein the R¹, R², R³ and R⁴ substituents are bonded to the steroid nucleus described for group 1 compounds, except that R¹ is in the β-configuration and double bonds are present at the 1-2 and 16-17 positions. Exemplary group 17 compound 1.2.4.1 is 3β,7β-dihydroxy-16-fluoro-17-aminoandrost-1,16-diene, 1.1.5.9 is 3β,17-dihydroxyandrost-1,16-diene, 1.1.7.1 is 3β-hydroxy-16-acetoxy-17-aminoandrost-1,16-diene and compound 1.1.4.10 is 3β-hydroxy-16-fluoro-17-acetoxyandrost-1,16-diene. Other exemplary group 17 compounds include 3β,17-dihydroxy-7β-acetoxyandrost-1,16-diene, 3β,17-dihydroxy-7β-methylandrost-1,16-diene, 3β,17-dihydroxy-7β-methoxyandrost-1,16-diene, 3β,7β,17-trihydroxyandrost-1,16-diene, 3β-amino-17-hydroxyandrost-1,16-diene, 3β-amino-7β,17-dihydroxyandrost-1,16-diene, 3β-hydroxy-17-aminoandrost-1,16-diene, 3β,7β-dihydroxy-17-aminoandrost-1,16-diene, 3β,17-dihydroxy-7β-aminoandrost-1,16-diene, 3β-hydroxy-7β,17-diacetylaminoandrost-1,16-diene, 3β-hydroxy-7β,17-dimethylaminoandrost-1,16-diene and 16-hydroxy, 16-methyl, 16-amino, 16-aminomethyl, 16-acetate and 16-halo analogs of any of these compounds.

Group 18. This group comprises compounds named in Table B having R¹, R², R³ and R⁴ substituents defined in Table A wherein the R¹, R², R³ and R⁴ substituents are bonded to the steroid nucleus described for group 1 compounds, except that R¹ is in the β-configuration, R^{10E} is in the β-configuration and double bonds are present at the 1-2 and 16-17 positions. Exemplary group 18 compound 1.2.4.1 is 3β,7β-dihydroxy-16-fluoro-17-amino-5β-androst-1,16-diene, 1.1.5.9 is 3β,17-dihydroxy-5β-androst-1,16-diene, 1.1.7.1 is 3β-hydroxy-16-acetoxy-17-amino-5β-androst-1,16-diene and compound 1.1.4.10 is 3β-hydroxy-16-fluoro-17-acetoxy-5β-androst-1,16-diene. Other exemplary group 18 compounds include 3β,17-dihydroxy-7β-acetoxy-5β-androst-1,16-diene, 3β,17-dihydroxy-7β-methyl-5β-androst-1,16-diene, 3β,17-dihydroxy-7β-methoxy-5β-androst-1,16-diene, 3β,7β,17-trihydroxy-5β-androst-1,16-diene, 3β-amino-17-hydroxy-5β-androst-1,16-diene, 3β-amino-7β,17-dihydroxy-5β-androst-1,16-diene, 3β-hydroxy-17-amino-5β-androst-1,16-diene, 3β,7β-dihydroxy-17-amino-5β-androst-1,16-diene, 3β,17-dihydroxy-7β-amino-5β-androst-1,16-diene, 3β-hydroxy-7β,17-diacetylamino-5β-androst-1,16-diene, 3β-hydroxy-7β,17-dimethylamino-5β-androst-1,16-diene and 16-hydroxy, 16-methyl, 16-amino, 16-aminomethyl, 16-acetate and 16-halo analogs of any of these compounds.

Group 19. This group comprises compounds named in Table B having R¹, R², R³ and R⁴ substituents defined in Table A wherein the R¹, R², R³ and R⁴ substituents are bonded to the steroid nucleus described for group 1 compounds, except that R¹ is in the β-configuration, R^{10F} and R^{10G} are absent and double bonds are present at the 1-2, 8-9 and 15-16 positions. Exemplary group 19 compounds include 1.2.4.1, which is 3β,7β-dihydroxy-16-fluoro-17β-aminoandrost-1,8(9),15-triene, 1.1.5.9, which is 3β,17β-dihydroxyandrost-1,8(9),15-triene, 1.1.7.1, which is 3β-hydroxy-16-acetoxy-17β-aminoandrost-1,8(9),15-triene and compound 1.1.4.10, which is 3β-hydroxy-16-fluoro-17β-acetoxyandrost-1,8(9),15-triene. Other exemplary group 19 compounds include 3β,17β-dihydroxy-7β-acetoxyandrost-1,8(9),15-triene, 3β,17β-dihydroxy-7β-methylandrost-1,8(9),15-triene, 3β,17β-dihydroxy-7β-methoxyandrost-1,8(9),15-triene, 3β,7β,17β-trihydroxyandrost-1,8(9),15-triene, 3β-amino-17β-hydroxyandrost-1,8(9),15-triene, 3β-amino-7β,17β-dihydroxyandrost-1,8(9),15-triene, 3β-hydroxy-17β-aminoandrost-1,8(9),15-triene, 3β,7β-dihydroxy-17β-aminoandrost-1,8(9),15-triene, 3β,17β-dihydroxy-7β-aminoandrost-1,8(9),15-triene, 3β-hydroxy-7β,17β-diacetylaminoandrost-1,8(9),15-triene, 3β-hydroxy-7β,17β-dimethylaminoandrost-1,8(9),15-triene and 16-hydroxy, 16-methyl, 16-amino, 16-aminomethyl, 16-acetate and 16-halo analogs of any of these compounds.

Group 20. This group comprises compounds named in Table B having R¹, R², R³ and R⁴ substituents defined in Table A wherein the R¹, R², R³ and R⁴ substituents are bonded to the steroid nucleus described for group 1 compounds, except that R¹ is in the β-configuration, R^{10E} is in the β-configuration, R^{10F} and R^{10G} are absent and double bonds are present at the 1-2, 8-9 and 15-16 positions. Exemplary group 20 compounds include 1.2.4.1, which is 3β,7β-dihydroxy-16-fluoro-17β-amino-5β-androst-1,8(9),15-triene, 1.1.5.9, which is 3β,17β-dihydroxy-5β-androst-1,8(9),15-triene, 1.1.7.1, which is 3β-hydroxy-16-acetoxy-17β-amino-5β-androst-1,8(9),15-triene and compound 1.1.4.10, which is 3β-hydroxy-16-fluoro-17β-acetoxy-5β-androst-1,8(9),15-triene. Other exemplary group 20 compounds include 3β,17β-dihydroxy-7β-acetoxy-5β-androst-1,8(9),15-triene, 3β,17β-dihydroxy-7β-methyl-5β-androst-1,8(9),15-triene, 3β,17β-dihydroxy-7β-methoxy-5β-androst-1,8(9),15-triene, 3β,7β,17β-trihydroxy-5β-androst-1,8(9),15-triene, 3β-amino-17β-hydroxy-5β-androst-1,8(9),15-triene, 3β-amino-7β,17β-dihydroxy-5β-androst-1,8(9),15-triene, 3β-hydroxy-17β-amino-5β-androst-1,8(9),15-triene, 3β,7β-dihydroxy-17β-amino-5β-androst-1,8(9),15-triene, 3β,17β-dihydroxy-7β-amino-5β-androst-1,8(9),15-triene, 3β,17β-dihydroxy-7β-aminoandrost-1,8(9),15-triene, 3β-hydroxy-7β,17β-diacetylamino-5β-androst-1,8(9),15-triene, 3β-hydroxy-7β,17β-dimethylamino-5β-androst-1,8(9),15-triene and 16-hydroxy, 16-methyl, 16-amino, 16-aminomethyl, 16-acetate and 16-halo analogs of any of these compounds.

Group 21. This group comprises compounds named in Table B having R¹, R², R³ and R⁴ substituents defined in Table A wherein the R¹, R², R³ and R⁴ substituents are bonded to the steroid nucleus described for group 1 compounds, except that R¹ is in the β-configuration, R^{10F} and R^{10H} are absent and double bonds are present at the 1-2, 8-14 and 15-16 positions. Exemplary group 21 compounds include 1.2.4.1, which is 3β,7β-dihydroxy-16-fluoro-17β-aminoandrost-1,8(14),15-triene, 1.1.5.9, which is 3β,17β-dihydroxyandrost-1,8(14),15-triene, 1.1.7.1, which is 3β-hydroxy-16-acetoxy-17β-aminoandrost-1,8(14),15-triene and compound 1.1.4.10, which is 3β-hydroxy-16-fluoro-17β-acetoxyandrost-1,8(14),15-triene. Other exemplary group 21 compounds include 3β,17β-dihydroxy-7β-acetoxyandrost-1,8(14),15-triene, 3β,17β-dihydroxy-7β-methylandrost-1,8(14),15-triene, 3β,17β-dihydroxy-7β-methoxyandrost-1,8(14),15-triene, 3β,7β,17β-trihydroxyandrost-1,8(14),15-triene, 3β-amino-17β-hydroxyandrost-1,8(14),15-triene, 3β-amino-7β,17β-dihydroxyandrost-1,8(14),15-triene, 3β-hydroxy-17β-aminoandrost-1,8(14),15-triene, 3β,7β-dihydroxy-17β-aminoandrost-1,8(14),15-triene, 3β,17β-dihydroxy-7β-aminoandrost-1,8(14),15-triene, 3β,17β-dihydroxy-7β-aminoandrost-1,8(9),15-triene, 3β-hydroxy-7β,17β-diacetylaminoandrost-1,8(14),15-triene, 3β-hydroxy-7β,17β-dimethylaminoandrost-1,8(14),15-triene and 16-hydroxy, 16-methyl, 16-amino, 16-aminomethyl, 16-acetate and 16-halo analogs of any of these compounds.

Group 22. This group comprises compounds named in Table B having R¹, R², R³ and R⁴ substituents defined in Table A wherein the R¹, R², R³ and R⁴ substituents are bonded to the steroid nucleus described for group 1 compounds, except that R¹ is in the β-configuration, R^{10E} is in the β-configuration, R^{10F} and R^{10H} are absent and double bonds are present at the 1-2, 8-14 and 15-16 positions. Exemplary group 22 compounds include 1.2.4.1, which is 3β,7β-dihydroxy-16-fluoro-17β-amino-5β-androst-1,8(14),15-triene, 1.1.5.9, which is 3β,17β-dihydroxy-5β-androst-1,8(14),15-triene, 1.1.7.1, which is 3β-hydroxy-16-acetoxy-17β-amino-5β-androst-1,8(14),15-triene and compound 1.1.4.10, which is 3β-hydroxy-16-fluoro-17β-acetoxy-5β-androst-1,8(14),15-triene. Other exemplary group 22 compounds include 3β,17β-dihydroxy-7β-acetoxy-5β-androst-1,8(14),15-triene, 3β,17β-dihydroxy-7β-methyl-5β-androst-1,8(14),15-triene, 3β,17β-dihydroxy-7β-methoxy-5β-androst-1,8(14),15-triene, 3β,7β,17β-trihydroxy-5β-androst-1,8(14),15-triene, 3β-amino-17β-hydroxy-5β-androst-1,8(14),15-triene, 3β-amino-7β,17β-dihydroxy-5β-androst-1,8(14),15-triene, 3β-hydroxy-17β-amino-5β-androst-1,8(14),15-triene, 3β,7β-dihydroxy-17β-amino-5β-androst-1,8(14),15-triene, 3β,17β-dihydroxy-7β-amino-5β-androst-1,8(14),15-triene, 3β-hydroxy-7β,17β-diacetylamino-5β-androst-1,8(14),15-triene, 3β-hydroxy-7β,17β-dimethylamino-5β-androst-1,8(14),15-triene and 16-hydroxy, 16-methyl, 16-amino, 16-aminomethyl, 16-acetate and 16-halo analogs of any of these compounds.

Group 23. This group comprises compounds named in Table B having R¹, R², R³ and R⁴ substituents defined in Table A wherein the R¹, R², R³ and R⁴ substituents are bonded to the steroid nucleus described for group 1 compounds, except that R¹ is in the β-configuration, R^{10E}, R^{10F} and R^{10H} are absent and double bonds are present at the 4-5, and 8-14 positions. Exemplary group 23 compounds include 1.2.4.1, which is 3β,7β-dihydroxy-16α-fluoro-17β-aminoandrost-4,8(14)-diene, 1.1.5.9, which is 3β,17β-dihydroxyandrost-4,8(14)-diene, 1.1.7.1, which is 3β-hydroxy-16α-acetoxy-17β-aminoandrost-4,8(14)-diene and compound 1.1.4.10, which is 3β-hydroxy-16α-fluoro-17β-acetoxyandrost-4,8(14)-diene. Other exemplary group 23 compounds include 3β,7β,17β-trihydroxyandrost-4,8(14)-diene, 3β,17β-dihydroxy-7β-methylandrost-4,8(14)-diene, 3β,17β-dihydroxy-7β-methoxyandrost-4,8(14)-diene, 3β,7β,17β-trihydroxyandrost-4,8(14)-diene, 3β-amino-17β-hydroxyandrost-4,8(14)-diene, 3β-amino-7β,17β-dihydroxyandrost-4,8(14)-diene, 3β-hydroxy-17β-aminoandrost-4,8(14)-diene, 3β,7β-dihydroxy-17β-aminoandrost-4,8(14)-diene, 3β,17β-dihydroxy-7β-aminoandrost-4,8(14)-diene, 3β-hydroxy-7β,17β-diacetylaminoandrost-1,8(14)-diene, 3β-hydroxy-7β,17β-dimethylaminoandrost-1,8(14)-diene and 16α-hydroxy, 16α-methyl, 16α-amino, 16α-aminomethyl, 16α-acetate and 16α-halo analogs of any of these compounds.

Group 24. This group comprises compounds named in Table B having R¹, R², R³ and R⁴ substituents defined in Table A wherein the R¹, R², R³ and R⁴ substituents are bonded to the steroid nucleus described for group 1 compounds, except that R¹ is in the β-configuration, R^{10E}, R^{10F} and R^{10G} are absent and double bonds are present at the 4-5, and 8-9 positions. Exemplary group 24 compounds include 1.2.4.1, which is 3β,7β-dihydroxy-16α-fluoro-17β-aminoandrost-4,8(9)-diene, 1.1.5.9, which is 3β,17β-dihydroxyandrost-4,8(9)-diene, 1.1.7.1, which is 3β-hydroxy-16α-acetoxy-17β-aminoandrost-4,8(9)-diene and compound 1.1.4.10, which is 3β-hydroxy-16α-fluoro-17β-acetoxyandrost-4,8(9)-diene. Other exemplary group 24 compounds include 3β,17β-dihydroxyandrost-4,8(9)-diene, 3β,7β,17β-trihydroxyandrost-4,8(9)-diene, 3β,17β-dihydroxy-7β-methylandrost-4,8(9)-diene, 3β,17β-dihydroxy-7β-methoxyandrost-4,8(9)-diene, 3β,7β,17β-trihydroxyandrost-4,8(9)-diene, 3β-amino-17β-hydroxyandrost-4,8(9)-diene, 3β-amino-7β,17β-dihydroxyandrost-4,8(9)-diene, 3β-hydroxy-17β-aminoandrost-4,8(9)-diene, 3β,7β-dihydroxy-17β-aminoandrost-4,8(9)-diene, 3β,17β-dihydroxy-7β-aminoandrost-4,8(9)-diene, 3β-hydroxy-7β,17β-diacetylaminoandrost-4,8(9)-diene, 3β-hydroxy-7β,17β-dimethylaminoandrost-4,8(9)-diene and 16α-hydroxy, 16α-methyl, 16α-amino, 16α-aminomethyl, 16α-acetate and 16α-halo analogs of any of these compounds.

Group 25. This group comprises compounds named in Table B having R¹, R², R³ and R⁴ substituents defined in Table A wherein the R¹, R², R³ and R⁴ substituents are bonded to the steroid nucleus described for group 1 compounds, except that double bonds are present at the 3-4, and 16-17 positions. Exemplary group 25 compounds include 1.2.4.1, which is 3,7β-dihydroxy-16-fluoro-17-aminoandrost-3,16-diene, 1.1.5.9, which is 3,17-dihydroxyandrost-3,16-diene, 1.1.7.1, which is 3-hydroxy-16-acetoxy-17-aminoandrost-3,16-diene and compound 1.1.4.10, which is 3-hydroxy-16-fluoro-17-acetoxyandrost-3,16-diene. Other exemplary group 25 compounds include 3,17-dihydroxyandrost-3,16-diene, 3,7β,17-trihydroxyandrost-3,16-diene, 3,17-dihydroxy-7β-methylandrost-3,16-diene, 3,17-dihydroxy-7β-methoxyandrost-3,16-diene, 3,7β,17-trihydroxyandrost-3,16-diene, 3-amino-17-hydroxyandrost-3,16-diene, 3-amino-7β,17-dihydroxyandrost-3,16-diene, 7β-amino-3,17-dihydroxyandrost-3,16-diene, 3-hydroxy-17-aminoandrost-3,16-diene, 3,7β-dihydroxy-17-aminoandrost-3,16-diene, 3-hydroxy-7β,17-diacetylaminoandrost-3,16-diene, 3-hydroxy-7β,17-dimethylaminoandrost-3,16-diene and 16-hydroxy, 16-methyl, 16-amino, 16-aminomethyl, 16-acetate and 16-halo analogs of any of these compounds.

Group 26. This group comprises compounds named in Table B having R¹, R², R³ and R⁴ substituents defined in Table A-wherein the R¹, R², R³ and R⁴ substituents are bonded to the steroid nucleus described for group 1 compounds, except that R^{10E} is present in the β-configuration and double bonds are present at the 3-4, and 16-17 positions. Exemplary group 26 compounds include 1.2.4.1, which is 3,7β-dihydroxy-16-fluoro-17-amino-5β-androst-3,16-diene, 1.1.5.9, which is 3,17-dihydroxy-5β-androst-3,16-diene, 1.1.7.1, which is 3-hydroxy-16-acetoxy-17-amino-5β-androst-3,16-diene and compound 1.1.4.10, which is 3-hydroxy-16-fluoro-17-acetoxy-5β-androst-3,16-diene. Other exemplary group 26 compounds include 3,17-dihydroxy-5β-androst-3,16-diene, 3,7β,17-trihydroxy-5β-androst-3,16-diene, 3,17-dihydroxy-7β-methyl-5β-androst-3,16-diene, 3,17-dihydroxy-7β-methoxy-5β-androst-3,16-diene, 3,7β,17-trihydroxy-5β-androst-3,16-diene, 3-amino-17-hydroxy-5β-androst-3,16-diene, 3-amino-7β,17-dihydroxy-5β-androst-3,16-diene, 3-hydroxy-17-amino-5β-androst-3,16-diene, 3,7β-dihydroxy-17-amino-5β-androst-3,16-diene, 3,17-dihydroxy-7β-amino-5β-androst-3,16-diene, 3-hydroxy-7β,17-diacetylamino-5β-androst-3,16-diene, 3-hydroxy-7β,17-dimethylamino-5β-androst-3,16-diene and 16-hydroxy, 16-methyl, 16-amino, 16-aminomethyl, 16-acetate and 16-halo analogs of any of these compounds.

Group 27. This group comprises compounds named in Table B having R¹, R², R³ and R⁴ substituents defined in Table A wherein the R¹, R², R³ and R⁴ substituents are bonded to the steroid nucleus described for group 1 compounds, except that double bonds are present at the 3-4, and 15-16 positions. Exemplary group 27 compounds include 1.2.4.1, which is 3,7β-dihydroxy-16-fluoro-17β-aminoandrost-3,15-diene, 1.1.5.9, which is 3,17β-dihydroxyandrost-3,15-diene, 1.1.7.1, which is 3-hydroxy-16-acetoxy-17β-aminoandrost-3,15-diene and compound 1.1.4.10, which is 3-hydroxy-16-fluoro-17β-acetoxyandrost-3,15-diene. Other exemplary group 27 compounds include 3,17β-dihydroxyandrost-3,15-diene, 3,7β,17β-trihydroxyandrost-3,15-diene, 3,17β-dihydroxy-7β-methylandrost-3,15-diene, 3,17β-dihydroxy-7β-methoxyandrost-3,15-diene, 3,7β,17β-trihydroxyandrost-3,15-diene, 3-amino-17β-hydroxyandrost-3,15-diene, 3-amino-7β,17β-dihydroxyandrost-3,15-diene, 3-hydroxy-17β-aminoandrost-3,15-diene, 3,7β-dihydroxy-17β-aminoandrost-3,15-diene, 3,17β-dihydroxy-7β-aminoandrost-3,15-diene, 3-hydroxy-7β,17β-diacetylaminoandrost-3,15-diene, 3-hydroxy-7β,17β-dimethylaminoandrost-3,15-diene and 16-hydroxy, 16-methyl, 16-amino, 16-aminomethyl, 16-acetate and 16-halo analogs of any of these compounds.

Group 28. This group comprises compounds named in Table B having R¹, R², R³ and R⁴ substituents defined in Table A wherein the R¹, R², R³ and R⁴ substituents are bonded to the steroid nucleus described for group 1 compounds, except that R^{10E} is present in the β-configuration and double bonds are present at the 3-4, and 15-16 positions. Exemplary group 28 compounds include 1.2.4.1, which is 3,7β-dihydroxy-16-fluoro-17β-amino-5β-androst-3,16-diene, 1.1.5.9, which is 3,17β-dihydroxy-5β-androst-3,16-diene, 1.1.7.1, which is 3-hydroxy-16-acetoxy-17β-amino-5β-androst-3,16-diene and compound 1.1.4.10, which is 3-hydroxy-16-fluoro-17β-acetoxy-5β-androst-3,16-diene. Other exemplary group 28 compounds include 3,7β,17β-trihydroxy-5β-androst-3,16-diene, 3,17β-dihydroxy-7β-methyl-5β-androst-3,16-diene, 3,17β-dihydroxy-7β-methoxy-5β-androst-3,16-diene, 3,7β,17β-trihydroxy-5β-androst-3,16-diene, 3-amino-17β-hydroxy-5β-androst-3,16-diene, 3-amino-7β,17β-dihydroxy-5β-androst-3,16-diene, 3-hydroxy-17β-amino-5β-androst-3,16-diene, 3,7β-dihydroxy-17β-amino-5β-androst-3,16-diene, 3,17β-dihydroxy-7β-amino-5β-androst-3,16-diene, 3-hydroxy-7β,17β-diacetylamino-5β-androst-3,15-diene, 3-hydroxy-7β,17β-dimethylamino-5β-androst-3,15-diene and 16-hydroxy, 16-methyl, 16-amino, 16-aminomethyl, 16-acetate and 16-halo analogs of any of these compounds.

Group 29. This group comprises compounds named in Table B having R¹, R², R³ and R⁴ substituents defined in Table A wherein the R¹, R², R³ and R⁴ substituents are bonded to the steroid nucleus described for group 1 compounds, except that R^{10E} and R⁶ are absent and double bonds are present at the 1-2, 3-4 and 5-10 positions, i.e., the A ring is aromatic. Exemplary group 29 compounds include 1.2.4.1, which is 3,7β-dihydroxy-16α-fluoro-17β-aminoandrost-1,3,5(10)-triene, 1.1.5.9, which is 3,17β-dihydroxyandrost-1,3,5(10)-triene, 1.1.7.1, which is 3-hydroxy-16β-acetoxy-17β-aminoandrost-1,3,5(10)-triene and compound 1.1.4.10, which is 3-hydroxy-16α-fluoro-17β-acetoxyandrost-1,3,5(10)-triene. Other exemplary group 29 compounds include 3,17β-dihydroxy-7β-acetoxyandrost-1,3,5(10)-triene, 3,17β-dihydroxy-7β-methylandrost-1,3,5(10)-triene, 3,17β-dihydroxy-7β-methoxyandrost-1,3,5(10)-triene, 3,7β,17β-trihydroxyandrost-1,3,5(10)-triene, 3-amino-17β-hydroxyandrost-1,3,5(10)-triene, 3-amino-7β,17β-dihydroxyandrost-1,3,5(10)-triene, 3-hydroxy-17β-aminoandrost-1,3,5(10)-triene, 3,7β-dihydroxy-17β-aminoandrost-1,3,5(10)-triene, 3,17β-dihydroxy-7β-aminoandrost-1,3,5(10)-triene, 3-hydroxy-7β,17β-diacetylaminoandrost-1,3,5(10)-triene, 3-hydroxy-7β,17β-dimethylaminoandrost-1,3,5(10)-triene and 16α-hydroxy, 16α-methyl, 16α-amino, 16α-aminomethyl, 16α-acetate and 16α-halo analogs of any of these compounds.

Group 30. This group comprises compounds named in Table B having R¹, R², R³ and R⁴ substituents defined in Table A wherein the R¹, R², R³ and R⁴ substituents are bonded to the steroid nucleus described for group 1 compounds, except that R¹ is in the β-configuration, R^{10E} is absent and double bonds are present at the 1-2, 4-5 and 6-7 positions. Exemplary group 30 compounds include 1.2.4.1, which is 3β,7-dihydroxy-16α-fluoro-17β-aminoandrost-1,4,6-triene, 1.1.5.9, which is 3β,17β-dihydroxyandrost-1,4,6-triene, 1.1.7.1, which is 3β-hydroxy-16α-acetoxy-17β-aminoandrost-1,4,6-triene and compound 1.1.4.10, which is 3β-hydroxy-16α-fluoro-17β-acetoxyandrost-1,4,6-triene. Other exemplary group 30 compounds include 3β,17β-dihydroxy-7-acetoxyandrost-1,4,6-triene, 3β,17β-dihydroxy-7-methylandrost-1,4,6-triene, 3,17β-dihydroxy-7-methoxyandrost-1,4,6-triene, 3β,7,17β-trihydroxyandrost-1,4,6-triene, 3β-amino-17β-hydroxyandrost-1,4,6-triene, 3β-amino-7,17β-dihydroxyandrost-1,4,6-triene, 3β-hydroxy-17β-aminoandrost-1,4,6-triene, 3β,7-dihydroxy-17β-aminoandrost-1,4,6-triene, 3,17β-dihydroxy-7β-aminoandrost-1,4,6-triene, 3β-hydroxy-7β,17β-diacetylaminoandrost-1,4,6-triene, 3β-hydroxy-7β,17β-dimethylaminoandrost-1,4,6-triene and 16α-hydroxy, 16α-methyl, 16α-amino, 16α-aminomethyl, 16α-acetate and 16α-halo analogs of any of these compounds.

Group 31. This group comprises compounds named in Table B having R¹, R², R³ and R⁴ substituents defined in Table A wherein the R¹, R², R³ and R⁴ substituents are bonded to the steroid nucleus described for group 1 compounds, except that R¹ is in the β-configuration, R^{10E} is absent and double bonds are present at the 1-2, 5-6 and 7-8 positions. Exemplary group 31 compounds include 1.2.4.1, which is 3β,7-dihydroxy-16α-fluoro-17β-aminoandrost-1,5,7-triene, 1.1.5.9, which is 3β,17β-dihydroxyandrost-1,5,7-triene, 1.1.7.1, which is 3β-hydroxy-16α-acetoxy-17β-aminoandrost-1,5,7-triene and compound 1.1.4.10, which is 3β-hydroxy-16α-fluoro-17β-acetoxyandrost-1,5,7-triene. Other exemplary group 31 compounds include 3β,17β-dihydroxy-7-acetoxyandrost-1,5,7-triene, 3β,17β-dihydroxy-7-methylandrost-1,5,7-triene, 3β,17β-dihydroxy-7-methoxyandrost-1,5,7-triene, 3β,7,17β-trihydroxyandrost-1,5,7-triene, 3β-amino-17βB-hydroxyandrost-1,5,7-triene, 3β-amino-7,17β-dihydroxyandrost-1,5,7-triene, 3β-hydroxy-17β-aminoandrost-1,5,7-triene, 3β,7-dihydroxy-17β-aminoandrost-1,5,7-triene, 3β,17β-dihydroxy-7β-aminoandrost-1,5,7-triene, 3β-hydroxy-7,17β-diacetylaminoandrost-1,5,7-triene, 3β-hydroxy-7,17β-dimethylaminoandrost-1,5,7-triene and 16α-hydroxy, 16α-methyl, 16α-amino, 16α-aminomethyl, 16α-acetate and 16α-halo analogs of any of these compounds.

Group 32. This group comprises compounds named in Table B having R¹, R², R³ and R⁴ substituents defined in Table A wherein the R¹, R², R³ and R⁴ substituents are bonded to the steroid nucleus described for group 1 compounds, except that R¹ is in the β-configuration, R^{10E} and R^{10F} are absent and double bonds are present at the 1-2, 5-6 and 15-16 positions. Exemplary group 32 compounds include 1.2.4.1, which is 3β,7β-dihydroxy-16-fluoro-17β-aminoandrost-1,5,15-triene, 1.1.5.9, which is 3β,17β-dihydroxyandrost-1,5,15-triene, 1.1.7.1, which is 3β-hydroxy-16-acetoxy-17β-aminoandrost-1,5,15-triene and compound 1.1.4.10, which is 3β-hydroxy-16-fluoro-17β-acetoxyandrost-1,5,15-triene. Other exemplary group 32 compounds include 3β,16-dihydroxy-17β-aminoandrost-1,5,15-triene, 3β,17β-dihydroxy-7β-acetoxyandrost-1,5,15-triene, 3β,17β-dihydroxy-7β-methylandrost-1,5,15-triene, 3β,17β-dihydroxy-7β-methoxyandrost-1,5,15-triene, 3β,7β,17β-trihydroxyandrost-1,5,15-triene, 3β-amino-17β-hydroxyandrost-1,5,15-triene, 3β-amino-7β,17β-dihydroxyandrost-1,5,15-triene, 3β-hydroxy-17β-aminoandrost-1,5,15-triene, 3β,7β-dihydroxy-17β-aminoandrost-1,5,15-triene, 3β,17β-dihydroxy-7β-aminoandrost-1,5,15-triene, 3β-hydroxy-7β,17β-diacetylaminoandrost-1,5,15-triene, 3β-hydroxy-7β,17β-dimethylaminoandrost-1,5,15-triene and 16-hydroxy, 16-methyl, 16-amino, 16-aminomethyl, 16-acetate and 16-halo analogs of any of these compounds.

Group 33. This group comprises compounds named in Table B having R¹, R², R³ and R⁴ substituents defined in Table A wherein the R¹, R², R³ and R⁴ substituents are bonded to the steroid nucleus described for group 1 compounds, except that R¹ is in the β-configuration, R^{10E} is absent and double bonds are present at the 1-2, 5-6 and 16-17 positions. Exemplary group 33 compounds include 1.2.4.1, which is 3β,7β-dihydroxy-16-fluoro-17-aminoandrost-1,5,16-triene, 1.1.5.9, which is 3β,17-dihydroxyandrost-1,5,16-triene, 1.1.7.1, which is 3β-hydroxy-16-acetoxy-17-aminoandrost-1,5,16-triene and compound 1.1.4.10, which is 3β-hydroxy-16-fluoro-17-acetoxyandrost-1,5,16-triene. Other exemplary group 33 compounds include 3β,16-dihydroxy-17-aminoandrost-1,5,16-triene, 3β,17-dihydroxy-7β-acetoxyandrost-1,5,16-triene, 3β,17-dihydroxy-7β-methylandrost-1,5,16-triene, 3β,17-dihydroxy-7β-methoxyandrost-1,5,16-triene, 3β,7β,17-trihydroxyandrost-1,5,16-triene, 3β-amino-17-hydroxyandrost-1,5,16-triene, 3β-amino-7β,17-dihydroxyandrost-1,5,16-triene, 3β-hydroxy-17-aminoandrost-1,5,16-triene, 3β,7β-dihydroxy-17-aminoandrost-1,5,16-triene, 3β,17β-dihydroxy-7β-aminoandrost-1,5,16-triene, 3β-hydroxy-7β,17-diacetylaminoandrost-1,5,16-triene, 3β-hydroxy-7β,17-dimethylaminoandrost-1,5,16-triene and 16-hydroxy, 16-methyl, 16-amino, 16-aminomethyl, 16-acetate and 16-halo analogs of any of these compounds.

Group 34. This group comprises compounds named in Table B having R¹, R², R³ and R⁴ substituents defined in Table A wherein the R¹, R², R³ and R⁴ substituents are bonded to the steroid nucleus described for group 1 compounds, except that R^{10E} and R⁶ are absent and double bonds are present at the 1-2, 3-4, 5-10 and 6-7 positions. Thus, for this group, the A ring is aromatic and a double bond is present at the 6-7 position. Exemplary group 34 compounds include 1.2.4.1, which is 3,7-dihydroxy-16α-fluoro-17β-aminoandrost-1,3,5(10),6-tetraene, 1.1.5.9, which is 3,17β-dihydroxyandrost-1,3,5(10),6-tetraene, 1.1.7.1, which is 3-hydroxy-16α-acetoxy-17β-aminoandrost-1,3,5(10),6-tetraene and compound 1.1.4.10, which is 3-hydroxy-16α-fluoro-17β-acetoxyandrost-1,3,5(10),6-tetraene. Other exemplary group 34 compounds include 3,17β-dihydroxy-7-acetoxyandrost-1,3,5(10),6-tetraene, 3,17β-dihydroxy-7-methylandrost-1,3,5(10),6-tetraene, 3,17β-dihydroxy-7-methoxyandrost-1,3,5(10),6-tetraene, 3,7,17β-trihydroxyandrost-1,3,5(10),6-tetraene, 3-amino-17β-hydroxyandrost-1,3,5(10),6-tetraene, 3-amino-7,17β-dihydroxyandrost-1,3,5(10),6-tetraene, 3-hydroxy-17β-aminoandrost-1,3,5(10),6-tetraene, 3,7β-dihydroxy-17β-aminoandrost-1,3,5(10),6-tetraene, 3,17β-dihydroxy-7β-aminoandrost-1,3,5(10),6-tetraene, 3-hydroxy-7,17β-diacetylaminoandrost-1,3,5(10),6-tetraene, 3-hydroxy-7,17β-dimethylaminoandrost-1,3,5(10),6-tetraene and 16α-hydroxy, 16α-methyl, 16α-amino, 16α-aminomethyl, 16α-acetate and 16α-halo analogs of any of these compounds.

Group 35. This group comprises compounds named in Table B having R¹, R², R³ and R⁴ substituents defined in Table A wherein the R¹, R², R³ and R⁴ substituents are bonded to the steroid nucleus described for group 1 compounds, except that R^{10E}, R^{10F} and R⁶ are absent and double bonds are present at the 1-2, 3-4, 5-10 and 7-8 positions. Thus, for this group, the A ring is aromatic and a double bond is present at the 7-8 position. Exemplary group 35 compounds include 1.2.4.1, which is 3,7-dihydroxy-16α-fluoro-17β-aminoandrost-1,3,5(10),7-tetraene, 1.1.5.9, which is 3,17β-dihydroxyandrost-1,3,5(10),7-tetraene, 1.1.7.1, which is 3-hydroxy-16α-acetoxy-17β-aminoandrost-1,3,5(10),7-tetraene and compound 1.1.4.10, which is 3-hydroxy-16α-fluoro-17-acetoxyandrost-1,3,5(10),7-tetraene. Other exemplary group 35 compounds include 3,17β-dihydroxy-7-acetoxyandrost-1,3,5(10), 7-tetraene, 3,17β-dihydroxy-7-methylandrost-1,3,5(10),7-tetraene, 3,17β-dihydroxy-7-methoxyandrost-1,3,5(10),7-tetraene, 3,7,17β-trihydroxyandrost-1,3,5(10),7-tetraene, 3-amino-17β-hydroxyandrost-1,3,5(10),7-tetraene, 3-amino-7,17β-dihydroxyandrost-1 ,3,5(10),7-tetraene, 3-hydroxy-17β-aminoandrost-1,3,5(10),7-tetraene, 3,7β-dihydroxy-17β-aminoandrost-1,3,5(10),7-tetraene, 3,17β-dihydroxy-7β-aminoandrost-1,3,5(10),7-tetraene, 3-hydroxy-7,17β-diacetylaminoandrost-1,3,5(10),7-tetraene, 3-hydroxy-7,17β-dimethylaminoandrost-1,3,5(10),7-tetraene and 16α-hydroxy, 16α-methyl, 16α-amino, 16α-aminomethyl, 16α-acetate and 16α-halo analogs of any of these compounds.

Group 36. This group comprises compounds named in Table B having R¹, R², R³ and R⁴ substituents defined in Table A wherein the R¹, R², R³ and R⁴ substituents are bonded to the steroid nucleus described for group 1 compounds, except that R^{10E}, R^{10F}, R^{10G} and R⁶ are absent and double bonds are present at the 1-2, 3-4, 5-10and 8-9 positions. Thus, for this group, the A ring is aromatic and a double bond is present at the 8-9 position. Exemplary group 36 compounds include 1.2.4.1, which is 3,7β-dihydroxy-16α-fluoro-17β-aminoandrost-1,3,5(10),8(9)-tetraene, 1.1.5.9, which is 3,17β-dihydroxyandrost-1,3,5(10),8(9)-tetraene, 1.1.7.1, which is 3-hydroxy-16α-acetoxy-17β-aminoandrost-1,3,5(10),8(9)-tetraene and compound 1.1.4.10, which is 3-hydroxy-16α-fluoro-17β-acetoxyandrost-1,3,5(10),8(9)-tetraene. Other exemplary group 36 compounds include 3,17β-dihydroxy-7β-acetoxyandrost-1,3,5(10),8(9)-tetraene, 3,17β-dihydroxy-7β-methylandrost-1,3,5(10),8(9)-tetraene, 3,17β-dihydroxy-7β-methoxyandrost-1,3,5(10),8(9)-tetraene, 3,7β,17β-trihydroxyandrost-1,3,5(10),8(9)-tetraene, 3-amino-17β-hydroxyandrost-1,3,5(10),8(9)-tetraene, 3-amino-7β,17β-dihydroxyandrost-1,3,5(10),8(9)-tetraene, 3-hydroxy-17β-aminoandrost-1,3,5(10),8(9)-tetraene, 3,7β-dihydroxy-17β-aminoandrost-1,3,5(10),8(9)-tetraene, 3,17β-dihydroxy-7β-aminoandrost-1,3,5(10),8(9)-tetraene, 3-hydroxy-7β,17β-diacetylaminoandrost-1,3,5(10),8(9)-tetraene, 3-hydroxy-7β,17β-dimethylaminoandrost-1,3,5(10),8(9)-tetraene and 16α-hydroxy, 16α-methyl, 16α-amino, 16α-aminomethyl, 16α-acetate and 16α-halo analogs of any of these compounds.

Group 37. This group comprises compounds named in Table B having R¹, R², R³ and R⁴ substituents defined in Table A wherein the R¹, R², R³ and R⁴ substituents are bonded to the steroid nucleus described for group 1 compounds, except that R^{10E}, R^{10F}, R^{10H} and R⁶ are absent and double bonds are present at the 1-2, 3-4, 5-10 and 8-14 positions. Thus, for this group, the A ring is aromatic and a double bond is present at the 8-14 position. Exemplary group 37 compounds include 1.2.4.1, which is 3,7β-dihydroxy-16α-fluoro-17β-aminoandrost-1,3,5(10),8(14)-tetraene, 1.1.5.9, which is 3,17β-dihydroxyandrost-1,3,5(10),8(14)-tetraene, 1.1.7.1, which is 3-hydroxy-16α-acetoxy-17β-aminoandrost-1,3,5(10),8(14)-tetraene and compound 1.1.4.10, which is 3-hydroxy-16α-fluoro-17β-acetoxyandrost-1,3,5(10),8(14)-tetraene. Other exemplary group 37 compounds include 3,17β-dihydroxy-7β-acetoxyandrost-1,3,5(10),8(14)-tetraene, 3,17β-dihydroxy-7β-methylandrost-1,3,5(10), 8(14)-tetraene, 3,17β-dihydroxy-7β-methoxyandrost-1,3,5(10),8(14)-tetraene, 3,7β,17β-trihydroxyandrost-1,3,5(10),8(14)-tetraene, 3-amino-17β-hydroxyandrost-1,3,5(10),8(14)-tetraene, 3-amino-7β,17β-dihydroxyandrost-1,3,5(10),8(14)-tetraene, 3-hydroxy-17β-aminoandrost-1,3,5(10),8(14)-tetraene, 3,7β-dihydroxy-17β-aminoandrost-1,3,5(10),8(14)-tetraene, 3,17β-dihydroxy-7β-aminoandrost-1,3,5(10),8(14)-tetraene, 3-hydroxy-7β,17β-diacetylaminoandrost-1,3,5(10),8(14)-tetraene, 3-hydroxy-7β,17β-dimethylaminoandrost-1,3,5(10),8(14)-tetraene and 16α-hydroxy, 16α-methyl, 16α-amino, 16α-aminomethyl, 16α-acetate and 16α-halo analogs of any of these compounds.

Group 38. This group comprises compounds named in Table B having R¹, R², R³ and R⁴ substituents defined in Table A wherein the R¹, R², R³ and R⁴ substituents are bonded to the steroid nucleus described for group 1 compounds, except that R^{10E} and R⁶ are absent and double bonds are present at the 1-2, 3-4, 5-10 and 15-16 positions. Thus, for this group, the A ring is aromatic and a double bond is present at the 15-16 position. Exemplary group 38 compounds include 1.2.4.1, which is 3,7β-dihydroxy-16-fluoro-17β-aminoandrost-1,3,5(10),15-tetraene, 1.1.5.9, which is 3,17β-dihydroxyandrost-1,3,5(10),15-tetraene, 1.1.7.1, which is 3-hydroxy-16-acetoxy-17β-aminoandrost-1,3,5(10),15-tetraene and compound 1.1.4.10, which is 3-hydroxy-16-fluoro-17β-acetoxyandrost-1,3,5(10),15-tetraene. Other exemplary group 38 compounds include 3,17β-dihydroxy-7β-acetoxyandrost-1,3,5(10),15-tetraene, 3,17β-dihydroxy-7β-methylandrost-1,3,5(10),15-tetraene, 3,17β-dihydroxy-7β-methoxyandrost-1,3,5(10),15-tetraene, 3,7β,17β-trihydroxyandrost-1,3,5(10),15-tetraene, 3-amino-17β-hydroxyandrost-1,3,5(10),15-tetraene, 3-amino-7β,17β-dihydroxyandrost-1,3,5(10),15-tetraene, 3-hydroxy-17β-aminoandrost-1,3,5(10),15-tetraene, 3,7β-dihydroxy-17β-aminoandrost-1,3,5(10),15-tetraene, 3,17β-dihydroxy-7β-aminoandrost-1,3,5(10),15-tetraene, 3-hydroxy-7β,17β-diacetylaminoandrost-1,3,5(10),15-tetraene, 3-hydroxy-7β,17β-dimethylaminoandrost-1,3,5(10),15-tetraene and 16-hydroxy, 16-methyl, 16-amino, 16-aminomethyl, 16-acetate and 16-halo analogs of any of these compounds.

Group 39. This group comprises compounds named in Table B having R¹, R², R³ and R⁴ substituents defined in Table A wherein the R¹, R², R³ and R⁴ substituents are bonded to the steroid nucleus described for group 1 compounds, except that R^{10E} and R⁶ are absent and double bonds are present at the 1-2, 3-4, 5-10 and 16-17 positions. Thus, for this group, the A ring is aromatic and a double bond is present at the 15-16 position. Exemplary group 39 compounds include 1.2.4.1, which is 3,7β-dihydroxy-16-fluoro-17-aminoandrost-1,3,5(10),16-tetraene, 1.1.5.9, which is 3,17-dihydroxyandrost-1,3,5(10),16-tetraene, 1.1.7.1, which is 3-hydroxy-16-acetoxy-17-aminoandrost-1,3,5(10),16-tetraene and compound 1.1.4.10, which is 3-hydroxy-16-fluoro-17-acetoxyandrost-1,3,5(10),16-tetraene. Other exemplary group 39 compounds include 3,17-dihydroxy-7β-acetoxyandrost-1,3,5(10),16-tetraene, 3,17-dihydroxy-7β-methylandrost-1,3,5(10),16-tetraene, 3,17-dihydroxy-7β-methoxyandrost-1,3,5(10),16-tetraene, 3,7β,17-trihydroxyandrost-1,3,5(10),16-tetraene, 3-amino-17-hydroxyandrost-1,3,5(10),16-tetraene, 3-amino-7β,17-dihydroxyandrost-1,3,5(10),16-tetraene, 3-hydroxy-17-aminoandrost-1,3,5(10),16-tetraene, 3,7β-dihydroxy-17-aminoandrost-1,3,5(10),16-tetraene, 3-hydroxy-7β,17-diacetylaminoandrost-1,3,5(10),16-tetraene, 3-hydroxy-7β,17-dimethylaminoandrost-1,3,5(10),16-tetraene and 16-hydroxy, 16-methyl, 16-amino, 16-aminomethyl, 16-acetate and 16-halo analogs of any of these compounds.

Group 40. This group comprises compounds named in Table B having R¹, R², R³ and R⁴ substituents defined in Table A wherein the R¹, R², R³ and R⁴ substituents are bonded to the steroid nucleus described for group 1 compounds, except that R^{10E} and R⁶ are absent and double bonds are present at the 1-2, 5-6, 7-8 and 15-16 positions. Thus, for this group, the A ring is aromatic and a double bond is present at the 15-16 position. Exemplary group 40 compounds include 1.2.4.1, which is 3β,7-dihydroxy-16-fluoro-17β-aminoandrost-1,5,7,15-tetraene, 1.1.5.9, which is 3β,17β-dihydroxyandrost-1,5,7,15-tetraene, 1.1.7.1, which is 3β-hydroxy-16-acetoxy-17β-aminoandrost-1,5,7,15-tetraene and compound 1.1.4.10, which is 3β-hydroxy-16-fluoro-17β-acetoxyandrost-1,5,7,15-tetraene. Other exemplary group 40 compounds include 3β,17β-dihydroxy-7-acetoxyandrost-1,5,7,15-tetraene, 3β,17β-dihydroxy-7-methylandrost-1,5,7,15-tetraene, 3β,17β-dihydroxy-7-methoxyandrost-1,5,7,15-tetraene, 3β,7,17β-trihydroxyandrost-1,5,7,15-tetraene, 3β-amino-17β-hydroxyandrost-1,5,7,15-tetraene, 3β-amino-7,17β-dihydroxyandrost-1,5,7,15-tetraene, 3β-hydroxy-17β-aminoandrost-1,5,7,15-tetraene, 3β,7-dihydroxy-17β-aminoandrost-1,5,7,15-tetraene, 3β-hydroxy-7,17β-diacetylaminoandrost-1,5,7,15-tetraene, 3β-hydroxy-7,17β-dimethylaminoandrost-1,5,7,15-tetraene and 16-hydroxy, 16-methyl, 16-amino, 16-aminomethyl, 16-acetate and 16-halo analogs of any of these compounds.

Group 41. This group comprises compounds named in Table B having R¹, R², R³ and R⁴ substituents defined in Table A wherein the R¹, R², R³ and R⁴ substituents are bonded to the steroid nucleus described for group 1 compounds, except that R¹ is in the β-configuration, R^{10G} is absent and double bonds are present at the 1-2 and 9-11 positions. Exemplary group 41 compounds include 1.2.4.1, which is 3β,7β-dihydroxy-16α-fluoro-17β-aminoandrost-1,9(11)-diene, 1. 1.5.9, which is 3β,17β-dihydroxyandrost-1,9(11)-diene, 1.1.7.1, which is 3β-hydroxy-16α-acetoxy-17β-aminoandrost-1,9(11)-diene and compound 1.1.4.10, which is 3β-hydroxy-16α-fluoro-17β-acetoxyandrost-1,9(11)-diene. Other exemplary group 41 compounds include 3β,17β-dihydroxy-7β-acetoxyandrost-1,9(11)-diene, 3β,17β-dihydroxy-7β-methylandrost-1,9(11)-diene, 3β,17β-dihydroxy-7β-methoxyandrost-1,9(11)-diene, 3β,7β,17β-trihydroxyandrost-1,9(11)-diene, 3β-amino-17β-hydroxyandrost-1,9(11)-diene, 3β-amino-7β,17β-dihydroxyandrost-1,9(11)-diene, 3β-hydroxy-17β-aminoandrost-1,9(11)-diene, 3β,7β-dihydroxy-17β-aminoandrost-1,9(11)-diene, 3,17β-dihydroxy-7β-aminoandrost-1,9(11)-diene, 3β-hydroxy-7β,17β-diacetylaminoandrost-1,9(11)-diene, 3β-hydroxy-7β,17β-dimethylaminoandrost-1,9(11)-diene and 16α-hydroxy, 16α-methyl, 16α-amino, 16α-aminomethyl, 16α-acetate and 16α-halo analogs of any of these compounds.

Group 42. This group comprises compounds named in Table B having R¹, R², R³ and R⁴ substituents defined in Table A wherein the R¹, R², R³ and R⁴ substituents are bonded to the steroid nucleus described for group 1 compounds, except that R¹ and R^{10E} are in the β-configuration, R^{10G} is absent and double bonds are present at the 1-2 and 9-11 positions. Exemplary group 42 compounds include 1.2.4.1, which is 3β,7β-dihydroxy-16α-fluoro-17β-amino-5β-androst-1,9(11)-diene, 1.1.5.9, which is 3β,17β-dihydroxy-5β-androst-1,9(11)-diene, 1.1.7.1, which is 3β-hydroxy-16α-acetoxy-17β-amino-5β-androst-1,9(11)-diene and compound 1.1.4.10, which is 3β-hydroxy-16α-fluoro-17β-acetoxy-5β-androst-1,9(11)-diene. Other exemplary group 42 compounds include 3β,17β-dihydroxy-7β-acetoxy-5β-androst-1,9(11)-diene, 3β,17β-dihydroxy-7β-methyl-5β-androst-1,9(11)-diene, 3β,17β-dihydroxy-7β-methoxy-5β-androst-1,9(11)-diene, 3β,7β,17β-trihydroxy-5β-androst-1,9(11)-diene, 3β-amino-17β-hydroxy-5β-androst-1,9(11)-diene, 3β-amino-7β,17β-dihydroxy-5β-androst-1,9(11)-diene, 3β-hydroxy-17β-amino-5β-androst-1,9(11)-diene, 3β,7β-dihydroxy-17β-amino-5β-androst-1,9(11)-diene, 3β,17β-dihydroxy-7β-amino-5β-androst-1,9(11)-diene, 3β-hydroxy-7β,17β-diacetylamino-5β-androst-1,9(11)-diene, 3β-hydroxy-7β,17β-dimethylamino-5β-androst-1,9(11)-diene and 16α-hydroxy, 16α-methyl, 16α-amino, 16α-aminomethyl, 16α-acetate and 16α-halo analogs of any of these compounds.

Group 43. This group comprises compounds named in Table B having R¹, R², R³ and R⁴ substituents defined in Table A wherein the R¹, R², R³ and R⁴ substituents are bonded to the steroid nucleus described for group 1 compounds, except that R¹ is in the β-configuration, R^{10E} and R^{10G} are absent and double bonds are present at the 1-2, 4-5 and 9-11 positions. Exemplary group 43 compounds include 1.2.4.1, which is 3β,7β-dihydroxy-16α-fluoro-17β-aminoandrost-1,4,9(11)-triene, 1.1.5.9, which is 3β,17β-dihydroxyandrost-1,4,9(11)-triene, 1.1.7.1, which is 3β-hydroxy-16α-acetoxy-17β-aminoandrost-1,4,9(11)-triene and compound 1.1.4.10, which is 3β-hydroxy-16α-fluoro-17β-acetoxyandrost-1,4,9(11)-triene. Other exemplary group 43 compounds include 3β,17β-dihydroxy-7β-acetoxyandrost-1,4,9(11)-triene, 3β,17β-dihydroxy-7β-methylandrost-1,4,9(11)-triene, 3β,17β-dihydroxy-7β-methoxyandrost-1,4,9(11)-triene, 3β,7β,17β-trihydroxyandrost-1,4,9(11)-triene, 3β-amino-17β-hydroxyandrost-1,4,9(11)-triene, 3β-amino-7β,17β-dihydroxyandrost-1,4,9(11)-triene, 3β-hydroxy-17β-aminoandrost-1,4,9(11)-triene, 3β,7β-dihydroxy-17β-aminoandrost-1,4,9(11)-triene, 3β,17β-dihydroxy-7β-aminoandrost-1,4,9(11)-triene, 3β-hydroxy-7β,17β-diacetylaminoandrost-1,4,9(11)-triene, 3β-hydroxy-7β,17β-dimethylaminoandrost-1,4,9(11)-triene and 16α-hydroxy, 16α-methyl, 16α-amino, 16α-aminomethyl, 16α-acetate and 16α-halo analogs of any of these compounds.

Group 44. This group comprises compounds named in Table B having R¹, R², R³ and R⁴ substituents defined in Table A wherein the R¹, R², R³ and R⁴ substituents are bonded to the steroid nucleus described for group 1 compounds, except that R¹ is in the β-configuration, R^{10E} and R^{10F} are absent and double bonds are present at the 5-6 and 7-8 positions. Exemplary group 44 compounds include 1.2.4.1, which is 3β,7-dihydroxy-16α-fluoro-17β-aminoandrost-5,7-diene, 1.1.5.9, which is 3β,17β-dihydroxyandrost-5,7-diene, 1.1.7.1, which is 3β-hydroxy-16α-acetoxy-17β-aminoandrost-5,7-diene and compound 1.1.4.10, which is 3β-hydroxy-16α-fluoro-17β-acetoxyandrost-5,7-diene. Other exemplary group 44 compounds include 3β,17β-dihydroxy-7β-acetoxyandrost-5,7-diene, 3β,17β-dihydroxy-7-methylandrost-5,7-diene, 3β,17β-dihydroxy-7-methoxyandrost-5,7-diene, 3β,7,17β-trihydroxyandrost-5,7-diene, 3β-amino-17β-hydroxyandrost-5,7-diene, 3β-amino-7,17β-dihydroxyandrost-5,7-diene, 3β-hydroxy-17β-aminoandrost-5,7-diene, 3β,7-dihydroxy-17β-aminoandrost-5,7-diene, 3β,17β-dihydroxy-7-aminoandrost-5,7-diene, 3β-hydroxy-7,17β-diacetylaminoandrost-5,7-diene, 3β-hydroxy-7,17β-dimethylaminoandrost-5,7-diene and 16α-hydroxy, 16α-methyl, 16α-amino, 16α-aminomethyl, 16α-acetate and 16α-halo analogs of any of these compounds.

Group 45. This group comprises compounds named in Table B having R¹, R², R³ and R⁴ substituents defined in Table A wherein the R¹, R², R³ and R⁴ substituents are bonded to the steroid nucleus described for group 1 compounds, except that R¹ is in the β-configuration, R^{10E}, R^{10G} and R⁶ are absent and double bonds are present at the 4-5 and 9-10 positions. Exemplary group 45 compounds include 1.2.4.1, which is 3β,7β-dihydroxy-16α-fluoro-17β-aminoandrost-4,9(10)-diene, 1.1.5.9, which is 3β,17β-dihydroxyandrost-4,9(10)-diene, 1.1.7.1, which is 3β-hydroxy-16α-acetoxy-17β-aminoandrost-4,9(10)-diene and compound 1.1.4.10, which is 3β-hydroxy-16-fluoro-17β-acetoxyandrost-4,9(10)-diene. Other exemplary group 45 compounds include 3β,17β-dihydroxy-7β-acetoxyandrost-4,9(10)-diene, 3β,17β-dihydroxy-7β-methylandrost-4,9(10)-diene, 3β,17β-dihydroxy-7β-methoxyandrost-4,9(10)-diene, 3β,7β,17β-trihydroxyandrost-4,9(10)-diene, 3β-amino-17β-hydroxyandrost-4,9(10)-diene, 3β-amino-7β,17β-dihydroxyandrost-4,9(10)-diene, 3β-hydroxy-17β-aminoandrost-4,9(10)-diene, 3β,7β-dihydroxy-17β-aminoandrost-4,9(10)-diene, 3β,17β-dihydroxy-7β-aminoandrost-4,9(10)-diene, 3β-hydroxy-7β,17β-diacetylaminoandrost-4,9(10)-diene, 3β-hydroxy-7β,17β-dimethylaminoandrost-4,9(10)-diene and 16α-hydroxy, 16α-methyl, 16α-amino, 16α-aminomethyl, 16α-acetate and 16α-halo analogs of any of these compounds.

Group 46. This group comprises compounds named in Table B having R¹, R², R³ and R⁴ substituents defined in Table A wherein the R¹, R², R³ and R⁴ substituents are bonded to the steroid nucleus described for group 1 compounds, except that R^{10E} and R⁶ are absent and double bonds are present at the 2-3 and 5-10 positions. Exemplary group 46 compounds include 1.2.4.1, which is 3,7β-dihydroxy-16α-fluoro-17β-aminoandrost-2,5(10)-diene, 1.1.5.9, which is 3,17β-dihydroxyandrost-2,5(10)-diene, 1.1.7.1, which is 3-hydroxy-16α-acetoxy-17β-aminoandrost-2,5(10)-diene and compound 1.1.4.10, which is 3-hydroxy-16α-fluoro-17β-acetoxyandrost-2,5(10)-diene. Other exemplary group 46 compounds include 3,17β-dihydroxy-7β-acetoxyandrost-2,5(10)-diene, 3,17β-dihydroxy-7β-methylandrost-2,5(10)-diene, 3,17β-dihydroxy-7β-methoxyandrost-2,5(10)-diene, 3,7β,17β-trihydroxyandrost-2,5(10)-diene, 3-amino-17β-hydroxyandrost-2,5(10)-diene, 3-amino-7β,17β-dihydroxyandrost-2,5(10)-diene, 3-hydroxy-17β-aminoandrost-2,5(10)-diene, 3,7β-dihydroxy-17β-aminoandrost-2,5(10)-diene, 3,17β-dihydroxy-7β-aminoandrost-2,5(10)-diene, 3-hydroxy-7β,17β-diacetylaminoandrost-2,5(10)-diene, 3-hydroxy-7β,17β-dimethylaminoandrost-2,5(10)-diene and 16α-hydroxy, 16α-methyl, 16α-amino, 16α-aminomethyl, 16α-acetate and 16α-halo analogs of any of these compounds.

Group 47. This group comprises compounds named in Table B having R¹, R², R³ and R⁴substituents defined in Table A wherein the R¹, R², R³ and R⁴ substituents are bonded to the steroid nucleus described for group 1 compounds, except that R¹ is in the β-configuration, R^{10E} and R⁶ are absent and a double bond is present at the 5-10 position. Exemplary group 47 compounds include 1.2.4.1, which is 3,7β-dihydroxy-16α-fluoro-17β-aminoandrost-5(10)-ene, 1.1.5.9, which is 3,17β-dihydroxyandrost-5(10)-ene, 1.1.7.1, which is 3-hydroxy-16α-acetoxy-17β-aminoandrost-5(10)-ene and compound 1.1.4.10, which is 3-hydroxy-16α-fluoro-17β-acetoxyandrost-5(10)-ene. Other exemplary group 47 compounds include 3,17β-dihydroxy-7β-acetoxyandrost-5(10)-ene, 3,17β-dihydroxy-7β-methylandrost-5(10)-ene, 3,17β-dihydroxy-7β-methoxyandrost-5(10)-ene, 3,7β,17β-trihydroxyandrost-5(10)-ene, 3-amino-17β-hydroxyandrost-5(10)-ene, 3-amino-7≢,17β-dihydroxyandrost-5(10)-ene, 3-hydroxy-17β-aminoandrost-5(10)-ene, 3,7β-dihydroxy-17β-aminoandrost-5(10)-ene, 3,17β-dihydroxy-7β-aminoandrost-5(10)-ene, 3-hydroxy-7β,17β-diacetylaminoandrost-5(10)-ene, 3-hydroxy-7β,17β-dimethylaminoandrost-5(10)-ene and 16α-hydroxy, 16α-methyl, 16α-amino, 16α-aminomethyl, 16α-acetate and 16α-halo analogs of any of these compounds.

Group 48. This group comprises compounds named in Table B having R¹, R², R³ and R⁴ substituents defined in Table A wherein the R¹, R², R³ and R⁴ substituents are bonded to the steroid nucleus described for group 1 compounds, except that R¹ is in the β-configuration, R^{10E} and R⁶ are absent and double bonds are present at the 5-10 and 15-16 positions. Exemplary group 48 compounds include 1.2.4.1, which is 3,7β-dihydroxy-16-fluoro-17β-aminoandrost-5(10),15-diene, 1.1.5.9, which is 3,17β-dihydroxyandrost-5(10),15-diene, 1.1.7.1, which is 3-hydroxy-16-acetoxy-17β-aminoandrost-5(10),15-diene and compound 1.1.4.10, which is 3-hydroxy-16-fluoro-17β-acetoxyandrost-5(10),15-diene. Other exemplary group 48 compounds include 3,17β-dihydroxy-7β-acetoxyandrost-5(10),15-diene, 3,17β-dihydroxy-7β-methylandrost-5(10),15-diene, 3,17β-dihydroxy-7β-methoxyandrost-5(10),15-diene, 3,7β,17β-trihydroxyandrost-5(10),15-diene, 3-amino-17β-hydroxyandrost-5(10),15-diene, 3-amino-7β,17β-dihydroxyandrost-5(10),15-diene, 3-hydroxy-17β-aminoandrost-5(10),15-diene, 3,7β-dihydroxy-17β-aminoandrost-5(10),15-diene, 3,17β-dihydroxy-7β-aminoandrost-5(10),15-diene, 3-hydroxy-7β,17β-diacetylaminoandrost-5(10),15-diene, 3-hydroxy-7β,17β-dimethylaminoandrost-5(10),15-diene and 16-hydroxy, 16-methyl, 16-amino, 16-aminomethyl, 16-acetate and 16-halo analogs of any of these compounds.

Group 49. This group comprises compounds named in Table B having R¹, R², R³ and R⁴ substituents defined in Table A wherein the R¹, R², R³ and R⁴ substituents are bonded to the steroid nucleus described for group 1 compounds, except that R¹ is in the β-configuration, R^{10E} and R⁶ are absent and double bonds are present at the 5-10 and 16-17 positions. Exemplary group 49 compounds include 1.2.4.1, which is 3β,7β-dihydroxy-16-fluoro-17-aminoandrost-5(10),16-diene, 1.1.5.9, which is 3β,17-dihydroxyandrost-5(10),16-diene, 1.1.7.1, which is 3β-hydroxy-16-acetoxy-17-aminoandrost-5(10),16-diene and compound 1.1.4.10, which is 3β-hydroxy-16-fluoro-17-acetoxyandrost-5(10),16-diene. Other exemplary group 49 compounds include 3β,17-dihydroxy-7β-acetoxyandrost-5(10),16-diene, 3β,17-dihydroxy-7β-methylandrost-5(10),16-diene, 3β,17-dihydroxy-7β-methoxyandrost-5(10),16-diene, 3β,7β,17-trihydroxyandrost-5(10),16-diene, 3β-amino-17-hydroxyandrost-5(10),16-diene, 3β-amino-7β,17-dihydroxyandrost-5(10),16-diene, 3β-hydroxy-17-aminoandrost-5(10),16-diene, 3β,7β-dihydroxy-17-aminoandrost-5(10),16-diene, 3β,17-dihydroxy-7β-aminoandrost-5(10),16-diene, 3β-hydroxy-7β,17-diacetylaminoandrost-5(10),16-diene, 3β-hydroxy-7β,17-dimethylaminoandrost-5(10),16-diene and 16-hydroxy, 16-methyl, 16-amino, 16-aminomethyl, 16-acetate and 16-halo analogs of any of these compounds.

Group 50. This group comprises compounds in compound groups 1-49 described above where no double bond is present at the 16-17 position, i.e., groups 1-3, 6-16, 19-24, 27-32, 34-38 and 40-48, and R⁴ is in the α-configuration instead of in the β-configuration. These compound groups are specified by adding group number 50- to the included group numbers. Thus, for example, compounds in group 50-1 are compounds in group 1 where R⁴ is in the α-configuration. Similarly, compounds in group 50-2 are compounds in group 2 where R⁴ is in the α-configuration and compounds in group 50-3 are compounds in group 3 where R⁴ is in the α-configuration. Other group 50 compound groups where R⁴ is in the α-configuration are defined in a similar manner and therefore are 50-6, 50-7, 50-8, 50-9, 50-10, 50-11, 50-12, 50-13, 50-14, 50-15, 50-16, 50-19, 50-20, 50-21, 50-22, 50-23, 50-24, 50-27, 50-28, 50-29, 50-30, 50-31, 50-32, 50-34, 50-35, 50-36, 50-37, 50-38, 50-40, 50-41, 50-42, 50-43, 50-44, 50-45, 50-46, 50-47 and 50-48. For each of these compound groups, compounds 1.1.1.1 through 10.10.10.10 in Table B specifies a compound as defined by the Table A substituents and the R⁴ α-configuration as specified in this group.

Exemplary group 50-1 compounds include 1.2.4.1, which is 3,7β-dihydroxy-16α-fluoro-17α-aminoandrost-1,3-diene, 1.1.5.9, which is 3,17α-dihydroxyandrost-1,3-diene, 1.1.6.1, which is 3,16α-dihydroxy-17α-aminoandrost-1,3-diene and 1.1.4.9, which is 3,17α-dihydroxy-16α-fluoroandrost-1,3-diene. Exemplary group 50-2 compounds include 1.2.4.1, which is 3,7β-dihydroxy-16α-fluoro-17α-amino-5β-androst-1,3-diene, 1.1.5.9, which is 3,17α-dihydroxy-5β-androst-1,3-diene, 1.1.6.1, which is 3,16α-dihydroxy-17α-amino-5β-androst-1,3-diene and 1.1.4.9, which is 3,17α-dihydroxy-16α-fluoro-5β-androst-1,3-diene. Exemplary group 50-3 compounds include 1.2.4.1, which is 3,7β-dihydroxy-16α-fluoro-17α-aminoandrost-1,3,5-triene, 1.1.5.9, which is 3,17α-dihydroxyandrost-1,3,5-triene, 1.1.6.1, which is 3,16α-dihydroxy-17α-aminoandrost-1,3,5-triene and 1.1.4.9, which is 3,17α-dihydroxy-16α-fluoroandrost-1,3,5-triene. Exemplary group 50-48 compounds include 1.2.4.1, which is 3β,7β-di-hydroxy-16α-fluoro-17α-aminoandrost-5(10),15-diene, 1.1.5.9, which is 3β,17α-dihydroxyandrost-5(10),15-diene, 1.1.6.1, which is 3β,16α-dihydroxy-17α-aminoandrost-5(10),15-diene and 1.1.4.9, which is 3β,17α-dihydroxy-16α-fluoroandrost-5(10),15-diene. Compounds in the other group 50 compound groups are specified or defined in an analogous manner.

Group 51. This group comprises compounds in compound groups 1-50 described above, wherein no double bond is present at the 2-3 or 3-4 positions and R¹ is in the α-configuration instead of in the β-configuration, i.e., groups 6 through 24, 30 through 33, 40 through 45, 47 through 49, 50-6 through 50-16, 50-19 through 50-24, 50-30 through 50-32, 50-40 through 50-45, 50-47 and 50-48. These compound groups are specified in a manner that is similar to that described for group 50, i.e., by adding group number 51- to the included group numbers. Thus, compounds in group 51-6 are compounds in group 6 where R¹ is in the α-configuration, compounds in group 51-7 are compounds in group 7 where R¹ is in the α-configuration, compounds in group 51-47 are compounds in group 47 where R¹ is in the α-configuration are compounds in group where R¹ is in the α-configuration, group 51-50-6 are compounds in group 50-6 where R¹ is in the α-configuration, group 51-50-7 are compounds in group 50-7 where R¹ is in the α-configuration, group 51-50-47 are compounds in group 50-47 where R¹ is in the α-configuration and group 51-50-48 are compounds in group 50-48 where R¹ is in the α-configuration. Other group 51 compound groups where R¹ is in the α-configuration are defined in a similar manner and therefore are 51-8, 51-9, 51-10, 51-11, 51-12, 51-13, 51-14, 51-15, 51-16, 51-17, 51-18, 51-19, 51-20, 51-21, 51-22, 51-23, 51-24, 51-30, 51-31, 51-32, 51-33, 51-40, 51-41, 51-42, 51-43, 51-44, 51-45, 51-47, 51-48, 51-49, 51-50-6, 51-50-7, 51-50-8, 51-50-9, 51-50-10, 51-50-11, 51-50-12, 51-50-13, 51-50-14, 51-50-15, 51-50-16, 51-50-19, 51-50-20, 51-50-21, 51-50-22, 51-50-23, 51-50-24, 51-50-30, 51-50-31, 51-50-32, 51-50-40, 51-50-41, 51-50-42, 51-50-43, 51-50-44, 51-50-45, 51-5--47 and 51-50-48. For each of these compound groups, compounds 1.1.1.1 through 10.10.10.10 in Table B specifies a compound as defined by the Table A substituents and the R¹ α-configuration as specified in this group.

Exemplary group 51-6 compounds include 1.2.4.1, which is 3α,7β-dihydroxy-16α-fluoro-17β-aminoandrost-1,5-diene, 1.1.5.9, which is 3α,17β-dihydroxyandrost-1,5-diene, 1.1.6.1, which is 3α,16α-dihydroxy-17β-aminoandrost-1,5-diene and 1.1.4.9, which is 3α,17β-dihydroxy-16α-fluoroandrost-1,5-diene. Exemplary group 51-7 compounds include 1.2.4.1, which is 3α,7-dihydroxy-16α-fluoro-17β-aminoandrost-1,6-diene, 1.1.5.9, which is 3α,17β-dihydroxyandrost-1,6-diene, 1.1.6.1, which is 3β,16β-dihydroxy-17β-aminoandrost-1,6-diene and 1.1.4.9, which is 3α,17β-dihydroxy-16α-fluoroandrost-1,6-diene. Exemplary group 51-50-47 compounds include 1.2.4.1, which is 3α,7β-dihydroxy-16α-fluoro-17α-aminoandrost-5(10)-ene, 1.1.5.9, which is 3α,17α-dihydroxyandrost-5(10)-ene, 1.1.6.1, which is 3α,16α-dihydroxy-17α-aminoandrost-5(10)-ene and 1.1.4.9, which is 3α,17α-dihydroxy-16α-fluoroandrost-5(10)-ene. Exemplary group 51-50-48 compounds include 1.2.4.1, which is 3α,7β-dihydroxy-16α-fluoro-17α-aminoandrost-5(10),15-diene, 1.1.5.9, which is 3α,17α-dihydroxyandrost-5(10),15-diene, 1.1.6.1, which is 3α,16α-dihydroxy-17α-aminoandrost-5(10),15-diene and 1.1.4.9, which is 3α,17α-dihydroxy-16α-fluoroandrost-5(10),15-diene. Compounds in the other group 51 compound groups are defined in an analogous manner.

Group 52. This group comprises compounds in compound groups 1-51 described above, wherein no double bond is present at the 15-16 or 16-17 positions and R³ is in the β-configuration instead of in the α-configuration, i.e., groups 1 through 3, 6 through 14, 23, 24, 29 through 37, 41 through 47, 50-1, 50-2, 50-3, 50-6 through 50-14, 50-23, 50-24, 50-29, 50-30, 50-31, 50-34 through 50-37, 50-41 through 50-47, 51-47, 51-6 through 51-14, 51-23, 51-24, 51-30, 51-31, 51-41 through 51-45 and 51-47. Compound groups in group 52 where R³ is in the β-configuration are 52-1, 52-2, 52-3, 52-6, 52-7, 52-8, 52-9, 52-10, 52-11, 52-12, 52-13, 52-14, 52-23, 52-24, 52-29, 52-30, 52-31, 52-32, 52-33, 52-34, 52-35, 52-36, 52-37, 52-41, 52-42, 52-43, 52-44, 52-45, 52-46, 52-47, 52-50-1, 52-50-2, 52-50-3, 52-50-6, 52-50-7, 52-50-8, 52-50-9, 52-50-10, 52-50-11, 52-50-12, 52-50-13, 52-50-14, 52-50-23, 52-50-24, 52-50-29, 52-50-30, 52-50-31, 52-50-34, 52-50-35, 52-50-36, 52-50-37, 52-50-41, 52-50-42, 52-50-43, 52-50-44, 52-50-45, 52-50-46, 52-50-47, 52-51-6, 52-51-7, 52-51-8, 52-51-9, 52-51-10, 52-51-11, 52-51-12, 52-51-13, 52-51-14, 52-51-23, 52-51-24, 52-51-30, 52-51-31, 52-51-41, 52-51-42, 52-51-43, 52-51-44, 52-51-45, 52-51-47, 52-51-50-6, 52-51-50-7, 52-51-50-8, 52-51-50-9, 52-51-50-10, 52-51-50-11, 52-51-50-12, 52-51-50-13, 52-51-50-14, 52-51-50-23, 52-51-50-24, 52-51-50-30, 52-51-50-31, 52-51-50-41, 52-51-50-42, 52-51-50-43, 52-51-50-44, 52-51-50-45 and 52-51-50-47. For each of these compound groups, compounds 1.1.1.1 through 10.10.10.10 in Table B specifies a compound as defined by the Table A substituents and the R³ β-configuration as specified in this group.

These compound groups are specified in a manner that is similar to that described for groups 50 and 51, i.e., by adding group number 52- to the included group numbers. Thus, for example, compounds in group 52-1 are compounds in group 1 where R³ is in the β-configuration, compounds in group 52-6 are compounds in group 6 where R³ is in the β-configuration, compounds in group 52-7 are compounds in group 7 where R³ is in the β-configuration compounds in group 52-50-1 are compounds in group 50-1 where R³ is in the β-configuration, compounds in group 52-51-50-6 are compounds in group 51-50-6 where R³ is in the β-configuration and group 52-51-50-47 are compounds in group 51-50-47 where R³ is in the β-configuration.

Exemplary group 52-6 compounds include 1.2.4.1, which is 3β,7β-dihydroxy-16β-fluoro-17β-aminoandrost-1,5-diene, 1.1.6.9, which is 3β,16β,17β-trihydroxyandrost-1,5-diene, 1.1.6.1, which is 3β,16β-dihydroxy-17β-aminoandrost-1,5-diene and 1.1.4.9, which is 3β,17β-dihydroxy-16β-fluoroandrost-1,5-diene. Exemplary group 52-50-7 compounds include 1.2.4.1, which is 3β,7-dihydroxy-16β-fluoro-17α-aminoandrost-1,6-diene, 1.1.6.9, which is 3β,16β,17α-dihydroxyandrost-1,6-diene, 1.1.6.1, which is 3β,16β-dihydroxy-17α-aminoandrost-1,6-diene and 1.1.4.9, which is 3β,17α-dihydroxy-16β-fluoroandrost-1,6-diene. Exemplary group 52-50-8 compounds include 1.2.4.1, which is 3β,7-dihydroxy-16β-fluoro-17α-amino-5β-androst-1,6-diene, 1.1.6.9, which is 3β,16β,17α-dihydroxy-5β-androst-1,6-diene, 1.1.6.1, which is 3β,16β-dihydroxy-17α-amino-5β-androst-1,6-diene and 1.1.4.9, which is 3β,17α-dihydroxy-16β-fluoro-5β-androst-1,6-diene. Exemplary group 52-51-7 compounds include 1.2.4.1, which is 3α,7-dihydroxy-16β-fluoro-17β-aminoandrost-1,6-diene, 1.1.6.9, which is 3α,16β,17β-dihydroxyandrost-1,6-diene, 1.1.6.1, which is 3α,16β-dihydroxy-17β-aminoandrost-1,6-diene and 1.1.4.9, which is 3α,17β-dihydroxy-16β-fluoroandrost-1,6-diene. Exemplary group 52-51-50-7 compounds include 1.2.4.1, which is 3α,7-dihydroxy-16β-fluoro-17α-aminoandrost-1,6-diene, 1.1.6.9, which is 3α,16β,17α-dihydroxyandrost-1,6-diene, 1.1.6.1, which is 3α,16β-dihydroxy-17α-aminoandrost-1,6-diene and 1.1.4.9, which is 3α,17α-dihydroxy-16β-fluoroandrost-1,6-diene. Exemplary group 52-51-47 compounds include 1.2.4.1, which is 3α,7β-dihydroxy-16β-fluoro-17β-aminoandrost-5(10)-ene, 1.1.6.9, which is 3α,16β,17β-dihydroxyandrost-5(10)-ene, 1.1.6.1, which is 3α,16β-dihydroxy-17β-aminoandrost-5(10)-ene and 1.1.4.9, which is 3α,17β-dihydroxy-16β-fluoroandrost-5(10)-ene. Exemplary group 52-51-50-47 compounds include 1.2.4.1, which is 3α,7β-dihydroxy-16β-fluoro-17α-aminoandrost-5(10)-ene, 1.1.6.9, which is 3α,16β,17α-dihydroxyandrost-5(10)-ene, 1.1.6.1, which is 3α,16β-dihydroxy-17α-aminoandrost-5(10)-ene and 1.1.4.9, which is 3α,17α-dihydroxy-16β-fluoroandrost-5(10)-ene. Compounds in the other group 52 compound groups are defined in an analogous manner.

Group 53. This group comprises compounds in the compound groups 1-52 described above, wherein R⁹ is a moiety other than —CH₂— or ═CH—. As is apparent from the moieties that R⁹ can be, compounds and genera of compounds are defined in this group. Exemplary R⁹ include —O—, —NH—, —NCH₃—, ═N—, —S—, —S(O)—, —S(O)(O)—, —S⁺(optionally substituted alkyl)-, —CHR¹⁰—, —C(R¹⁰)₂— or ═CR¹⁰— where R¹⁰ are independently selected and a single R¹⁰ can be in the α-configuration or the β-configuration. When one or both R¹⁰ are not —H, exemplary R⁹ include —CH(α-OH)—, —CH(β-OH)—, —C(β-CH₃)(α-OH)—, —C(α-CH₃)(β-OH)—, —CH(α-C1-6 ester)-, —CH(β-C1-6 ester)-, —CH(α-O—C1-6 alkyl)-, —CH(β-O—C1-6 alkyl)-, —CH(α-S—C1-6 alkyl)-, —CH(β-S—C1-6 alkyl)-, —CH(α-NH—C1-6 alkyl)-, —CH(β-NH—C1-6 alkyl)-, —CH(α-O—C2-6 alkenyl)-, —CH(β-O—C2-6 alkenyl)-, —CH(α-O—C2-6 alkynyl)-, —CH(β-O—C2-6 alkynyl)-, —CH(α-O—C1-6 alkoxy)-, —CH(β-O—C1-6 alkoxy)-, —CH(α-O—CH₂—C2-6 alkenyl)-, —CH(β-O—CH₂—C2-6 alkenyl)-, —CH(α-O—CH₂—C2-6 alkynyl)-, —CH(β-O—CH₂—C2-6 alkynyl)-, —CH(α-C-linked heterocycle)-, —CH(β-C-linked heterocycle)-, —CH(α-N-linked heterocycle)-, —CH(β-N-linked heterocycle)-, —CH(α-halogen)-, —CH(β-halogen)-, —C(F)₂—, —C(CI)₂—, —C(Br)₂—, —C(I)₂—, —C(CH₃)₂—, —C(C₂H₅)₂—, —CH(α-SH)—, —CH(β-SH)—, —CH(α-NH₂)—, —CH(β-NH₂)—, —CH(α-NHCH₃)—, —CH(β-NHCH₃)—, —CH(α-N[CH₃]₂)—, —CH(β-N[CH₃]₂)—, —CH(α-N[C₂H₅]₂)—, —CH(β-N[C₂H₅]₂)—, —CH(α-NO₂)—, —CH(β-NO₂)—, —CH(α-N₃)—, —CH(β-N₃)—, —CH(α-CN)—, —CH(β-CN)—, —CH(α-SCN)—, —CH(β-SCN)—, —C(β-CH₃)(α-CN)—, —C(α-CH₃)(β-CN)—, —CH(α-NC(O)—(CH₂)ₘ—CH₃)—, —CH(β-NC(O)—(CH₂)ₘ—CH₃)—, —CH(α-NC(O)O—(CH₂)ₘ—CH₃)—, —CH(β-NC(O)O—(CH₂)ₘ—CH₃)—, —C(C1-4 alkyl)₂, —C(C1-4 alkenyl)₂-, where m is 0, 1, 2, 3, 4, 5 or 6, and any alkyl, alkenyl, alkynyl, alkoxy, alkenyloxy, alkynyloxy or heterocycle moiety is optionally substituted and each is independently chosen. When no double bond is present at the 1-2 or 2-3 positions, R⁹ can be —O—, —NH— or —S—, or it can be linked to a double bonded R¹⁰ moiety such as ═O, ═S, ═NOH, ═NCH₃, ═NH, ═CH₂, ═CH₂CH₃, ═CH₂CH₂OH, ═CH₂C(O)OH or another moiety as defined herein for R¹⁰. In these cases, R⁹ is a moiety such as —C(O)—, —C(NOH)— or —C(═CH₂)—. When a double bond is present at the 1-2 or 2-3 positions, R⁹ can be ═N—. In other embodiments, R⁹ is absent, leaving a 5-membered ring.

Groups of compounds in this group are defined essentially as described above for groups 50, 51 and 52. Compound groups in group 53 where R⁹ is substituted or is absent thus include 53-1, 53-2, 53-3, 53-4, 53-5, 53-6, 53-7, 53-8, 53-9, 53-10, 53-11, 53-12, 53-13, 53-14, 53-15, 53-16, 53-17, 53-18, 53-19, 53-20, 53-21, 53-22, 53-23, 53-24, 53-25, 53-26, 53-27, 53-28, 53-29, 53-30, 53-31, 53-32, 53-33, 53-34, 53-35, 53-36, 53-37, 53-38, 53-39, 53-40, 53-41, 53-42, 53-43, 53-44, 53-45, 53-46, 53-47, 53-48, 53-49, 53-51-6, 53-51-7, 53-51-8, 53-51-9, 53-51-10, 53-51-11, 53-51-12, 53-51-13, 53-51-14, 53-51-15, 53-51-16, 53-51-17, 53-51-18, 53-51-19, 53-51-20, 53-51-21, 53-51-22, 53-51-23, 53-51-24, 53-51-30, 53-51-31, 53-51-32, 53-51-33, 53-51-40, 53-51-41, 53-51-42, 53-51-43, 53-51-44, 53-51-45, 53-51-47, 53-51-48, 53-51-49, 53-51-50-6, 53-51-50-7, 53-51-50-8, 53-51-50-9, 53-51-50-10, 53-51-50-11, 53-51-50-12, 52-51-50-13, 53-51-50-14, 53-51-50-15, 53-51-50-16, 53-51-50-19, 53-51-50-20, 53-51-50-21, 53-51-50-22, 53-51-50-23, 53-51-50-24, 53-51-50-30, 53-51-50-31, 53-51-50-32, 53-51-50-40, 53-51-50-41, 53-51-50-42, 53-51-50-43, 53-51-50-44, 53-51-50-45, 53-51-50-47, 53-51-50-48, 53-52-1, 53-52-2, 53-52-3, 53-52-6, 53-52-7, 53-52-8, 53-52-9, 53-52-10, 53-52-11, 53-52-12, 53-52-13, 53-52-14, 53-52-23, 53-52-24, 53-52-29, 53-52-30, 53-52-31, 53-52-32, 53-52-33, 53-52-34, 53-52-35, 53-52-36, 53-52-37, 53-52-41, 53-52-42, 53-52-43, 53-52-44, 53-52-45, 53-52-46, 53-52-47, 53-52-50-1, 53-52-2, 53-52-50-3, 53-52-50-6, 53-52-50-7, 53-52-50-8, 53-52-50-9, 53-52-50-10, 53-52-50-11, 53-52-50-12, 53-52-50-13, 53-52-50-14, 53-52-50-23, 53-52-50-24, 53-52-50-29, 53-52-50-30, 53-52-50-31, 53-52-50-34, 53-52-50-35, 53-52-50-36, 53-52-50-37, 53-52-50-41, 53-52-50-42, 53-52-50-43, 53-52-50-44, 53-52-50-45, 53-52-50-46, 53-52-50-47, 53-52-51-6, 53-52-51-7, 53-52-51-8, 53-52-51-9, 53-52-51-10, 53-52-51-11, 53-52-51-12, 53-52-51-13, 53-52-51-14, 53-52-51-23, 53-52-51-24, 53-52-51-30, 53-52-51-31, 53-52-51-41, 53-52-51-42, 53-52-51-43, 53-52-51-44, 53-52-51-45, 53-52-51-47, 53-52-51-50-6, 53-52-51-50-7, 53-52-51-50-8, 53-52-51-50-9, 53-52-51-50-10, 53-52-51-50-11, 53-52-51-50-12, 53-52-51-50-13, 53-52-51-50-14, 53-52-51-50-23, 53-52-51-50-24, 53-52-51-50-30, 53-52-51-50-31, 53-52-51-50-41, 53-52-51-50-42, 53-52-51-50-43, 53-52-51-50-44, 53-52-51-50-45 and 53-52-51-50-47. For each of these compound groups, designations 1.1.1.1 through 10.10.10.10 in Table B specifies a compound or genus of compounds as defined by the Table A substituents and any R⁹ moiety as described here or elsewhere herein.

Exemplary compounds in group 53-44 when R⁹ is —O— include compound 1.2.4.1, which is 2-oxa-3β,7β-dihydroxy-16α-fluoro-17β-aminoandrost-5,7-diene, 1.1.5.9, which is 2-oxa-3β,17β-dihydroxyandrost-5,7-diene, 1.1.6.9, which is 2-oxa-3β,16,17β-trihydroxyandrost-5,7-diene, 1.1.6.1, which is 2-oxa-3β,16α-dihydroxy-17β-aminoandrost-5,7-diene and 1.1.4.9, which is 2-oxa-3β,17β-dihydroxy-16α-fluoroandrost-5,7-diene. Exemplary compounds in group 53-44 when R⁹ is —NH— include compound 1.2.4.1, which is 2-aza-3β,7β-dihydroxy-16α-fluoro-17β-aminoandrost-5,7-diene, 1.1.5.9, which is 2-aza-3β,17β-dihydroxyandrost-5,7-diene, 1.1.6.9, which is 2-aza-3β,16α,17β-trihydroxyandrost-5,7-diene, 1.1.6.1, which is 2-aza-3β,16α-dihydroxy-17β-aminoandrost-5,7-diene and 1.1.4.9, which is 2-aza-3β,17β-dihydroxy-16α-fluoroandrost-5,7-diene. Exemplary compounds in group 53-44 when R⁹ is —S— include compound 1.2.4.1, which is 2-thia-3β,7β-dihydroxy-16α-fluoro-17β-aminoandrost-5,7-diene, 1.1.5.9, which is 2-thia-3β,17β-dihydroxyandrost-5,7-diene, 1.1.6.9, which is 2-thia-3β,16α,17β-trihydroxyandrost-5,7-diene, 1.1.6.1, which is 2-thia-3α,16α-dihydroxy-17β-aminoandrost-5,7-diene and 1.1.4.9, which is 2-thia-3β,17β-dihydroxy-16α-fluoroandrost-5,7-diene. Exemplary compounds in group 53-44 when R⁹ is —CH(α-NH[CH₃])— include compound 1.2.4.1, which is 2α-methylamino-3β,7β-dihydroxy-16α-fluoro-17β-aminoandrost-5,7-diene, 1.1.5.9, which is 2α-methylamino-3β,17β-dihydroxyandrost-5,7-diene, 1.1.6.9, which is 2α-methylamino-3β,16α,17β-trihydroxyandrost-5,7-diene, 1.1.6.1, which is 2α-methylamino-3β,16α-dihydroxy-17β-aminoandrost-5,7-diene and 1.1.4.9, which is 2α-methylamino-3β,17β-dihydroxy-16α-fluoroandrost-5,7-diene. Exemplary compounds in group 53-44 when R⁹ is —CH(α-OH)— include compound 1.2.4.1, which is 2α,3β,7β-trihydroxy-16α-fluoro-17β-aminoandrost-5,7-diene, 1.1.5.9, which is 2α,3β,17β-trihydroxyandrost-5,7-diene, 1.1.6.9, which is 2α,3β,16α,17β-tetrahydroxyandrost-5,7-diene, 1.1.6.1, which is 2α,3β,16α-trihydroxy-17β-aminoandrost-5,7-diene and 1.1.4.9, which is 2α,3β,17β-trihydroxy-16α-fluoroandrost-5,7-diene. Exemplary compounds in group 53-44 when R⁹ is —CH(α-OCH₃)— include compound 1.2.4.1, which is 2α-methoxy-3β,7β-dihydroxy-16α-fluoro-17β-aminoandrost-5,7-diene, 1.1.5.9, which is 2α-methoxy-3β,17β-dihydroxyandrost-5,7-diene, 1.1.6.9, which is 2α-methoxy-3β,16α,17β-trihydroxyandrost-5,7-diene, 1.1.6.1, which is 2α-methoxy-3β,16α-dihydroxy-17β-aminoandrost-5,7-diene and 1.1.4.9, which is 2α-methoxy-3β,17β-dihydroxy-16α-fluoroandrost-5,7-diene. Exemplary compounds in group 53-44 when R9 is —CH(β-OC(O)CH₃)— include compound 1.2.4.1, which is 2β-acetoxy-3β,7β-dihydroxy-16α-fluoro-17β-aminoandrost-5,7-diene, 1.1.5.9, which is 2β-acetoxy-3β,17β-dihydroxyandrost-5,7-diene, 1.1.6.9, which is 2β-acetoxy-3β,16α,17β-trihydroxyandrost-5,7-diene, 1.1.6.1, which is 2β-acetoxy-3β,16α-dihydroxy-17β-aminoandrost-5,7-diene and 1.1.4.9, which is 2β-acetoxy-3β,17β-dihydroxy-16α-fluoroandrost-5,7-diene. Exemplary compounds in group 53-50-44 when R⁹ is —O— include compound 1.2.4.1, which is 2-oxa-3β,7β-dihydroxy-16α-fluoro-17α-aminoandrost-5,7-diene, 1.1.5.9, which is 2-oxa-3β,17α-dihydroxyandrost-5,7-diene, 1.1.6.9, which is 2-oxa-3β,16α,17α-trihydroxyandrost-5,7-diene, 1.1.6.1, which is 2-oxa-3β,16α-dihydroxy-17α-aminoandrost-5,7-diene and 1.1.4.9, which is 2-oxa-3β,17α-dihydroxy-16α-fluoroandrost-5,7-diene. Exemplary compounds in group 53-51-44 when R⁹ is —O— include compound 1.2.4.1, which is 2-oxa-3α,7β-dihydroxy-16α-fluoro-17β-aminoandrost-5,7-diene, 1.1.5.9, which is 2-oxa-3α,17β-dihydroxyandrost-5,7-diene, 1.1.6.9, which is 2-oxa-3,16α,17β-trihydroxyandrost-5,7-diene, 1.1.6.1, which is 2-oxa-3α,16α-dihydroxy-17β-aminoandrost-5,7-diene and 1.1.4.9, which is 2-oxa-3α,17β-dihydroxy-16α-fluoroandrost-5,7-diene. Exemplary compounds in group 53-51-50-44 when R⁹ is —O— include compound 1.2.4.1, which is 2-oxa-3α,7β-dihydroxy-16α-fluoro-17α-aminoandrost-5,7-diene, 1.1.5.9, which is 2-oxa-3α,17α-dihydroxyandrost-5,7-diene, 1.1.6.9, which is 2-oxa-3α,16α,17α-trihydroxyandrost-5,7-diene, 1.1.6.1, which is 2-oxa-3α,16α-dihydroxy-17α-aminoandrost-5,7-diene and 1.1.4.9, which is-2-oxa-3α,17α-dihydroxy-16α-fluoroandrost-5,7-diene. Compounds or genera of compounds in the other group 53 compound groups where R⁹ is a moiety described here or elsewhere herein are defined as described in Tables A and B in the same manner.

Group 54. This group comprises compounds and compound genera in compound groups 1-53 described above, wherein R⁸ is a moiety other than —CH₂— or ═CH—. Exemplary R⁸ include —O—, —NH—, —NCH₃—, ═N—, —S—, —S(O)—, —S(O)(O)—, —CHR¹⁰—, —C(R¹⁰)₂— or ═CR¹⁰— where R¹⁰ are independently selected and each R¹⁰ can be in the α-configuration or the β-configuration. When one or both R¹⁰ are not —H, exemplary R⁸ include —O—, —NH—, —NCH₃—, ═N—, —S—, —S(O)—, —S(O)(O)—, —S⁺(optionally substituted alkyl)-, —CHR¹⁰—, —C(R¹⁰)₂— or ═CR¹⁰— where R¹⁰ are independently selected and a single R¹⁰ can be in the α-configuration or the β-configuration. When one or both R¹⁰ are not —H, exemplary R⁹ include —CH(α-OH)—, —CH(β-OH)—, —CH(α-C1-6 ester)-, —CH(β-C1-6 ester)-, —CH(α-O—C1-6 alkyl)-, —CH(βO—C1-6 alkyl)-, —CH(α-S—C1-6 alkyl)-, —CH(β-S—C1-6 alkyl)-, —CH(α-NH-C1-6 alkyl)-, —CH(β-NH—C1-6 alkyl)-, —CH(α-O—C2-6 alkenyl)-, —CH(β-O—C2-6 alkenyl)-, —CH(α-O—C2-6 alkynyl)-, —CH(β-O—C2-6 alkynyl)-, —CH(α-O—C1-6 alkoxy)-, —CH(β-O—C1-6 alkoxy)-, —CH(α-O—CH₂-C2-6 alkenyl)-, —CH(β-O—CH₂-C2-6 alkenyl)-, —CH(α-O—CH₂-C2-6 alkynyl)-, —CH(β-O—CH₂-C2-6 alkynyl)-, —CH(α-C-linked heterocycle)-, —CH(β-C-linked heterocycle)-, —CH(α-N-linked heterocycle)-, —CH(β-N-linked heterocycle)-, —CH(α-halogen)-, —CH(β-halogen)-, —C(F)₂—, —C(Cl)₂—, —C(Br)₂—, —C(I)₂—, —C(CH₃)₂—, —C(C₂H₅)₂—, —CH(α-SH)—, —CH(β-SH)—, —CH(α-NH₂)—, —CH(β-NH₂)—, —CH(α-NHCH₃)—, —CH(β-NHCH₃)—, —CH(α-N[CH₃]₂)—, —CH(β-N[CH₃]₂)—, —CH(α-N[C₂H₅]₂)—, —CH(β-N[C_{C}₂H₅]₂)—, —CH(α-NO₂)—, —CH(β-NO₂)—, —CH(α-N₃)—, —CH(β-N₃)—, —CH(α-CN)—, —CH(β-CN)—, —CH(α-SCN)—, —CH(β-SCN)—, —CH(α-NC(O)—(CH₂)ₘ—CH₃)—, —CH(β-NC(O)—(CH₂)ₘ—CH₃)—, —CH(α-NC(O)O—(CH₂)ₘ—CH₃)—, —CH(β-NC(O)O—(CH₂)ₘCH₃)—, —C(C1-4 alkyl)₂—, —C(C1-4 alkenyl)₂-, where m is 0, 1, 2, 3, 4, 5 or 6, and any alkyl, alkenyl, alkynyl, alkoxy, alkenyloxy, alkynyloxy or heterocycle moiety is optionally substituted and each is independently chosen. When no double bond is present at the 9-11 position, R⁸ can be a ═N—, —O— or —S-heteroatom, or R⁸ can be linked to a double bonded R¹⁰ moiety such as ═O, ═S, ═NOH, ═NCH₃, ═NH, ═CH₂, ═CH₂CH₃, ═CH₂CH₂-halogen, ═CH₂CH₂OH, ═CH₂C(O)OH or another moiety as defined herein for R¹⁰. In these cases, R⁸ is a moiety such as —C(O)—, —C(NOH)— or —C(═CH₂)—. When a double bond is present at the 9-11 position, R⁸ can be ═N—. In other embodiments, R⁸ is absent, leaving a 5-membered ring.

Groups of compounds in this group are defined essentially as described above, e.g., for groups 52 and 53. Compound groups in group 54 where R⁸ is substituted or is absent thus include 54-1, 54-2, 54-3, 54-4, 54-5, 54-6, 54-7, 54-8, 54-9, 54-10, 54-11, 54-12, 54-13, 54-14, 54-15, 54-16, 54-17, 54-18, 54-19, 54-20, 54-21, 54-22, 54-23, 54-24, 54-25, 54-26, 54-27, 54-28, 54-29, 54-30, 54-31, 54-32, 54-33, 54-34, 54-35, 54-36, 54-37, 54-38, 54-39, 54-40, 54-41, 54-42, 54-43, 54-44, 54-45, 54-46, 54-47, 54-48, 54-49, 54-50-1, 54-50-2, 54-50-3, 54-50-6, 54-50-7, 54-50-8, 54-50-9, 54-50-10, 54-50-11, 54-50-12, 54-50-13, 54-50-14, 54-50-15, 54-50-16, 54-50-19, 54-50-20, 54-50-21, 54-50-22, 54-50-23, 54-50-24, 54-50-27, 54-50-28, 54-50-29, 54-50-30, 54-50-31, 54-50-32, 54-50-34, 54-50-35, 54-50-36, 54-50-37, 54-50-38, 54-50-40, 54-50-41, 54-50-42, 54-50-43, 54-50-44, 54-50-45, 54-50-46, 54-50-47, 54-50-48, 54-51-6, 54-51-7, 54-51-8, 54-51-9, 54-51-10, 54-51-11, 54-51-12, 54-51-13, 54-51-14, 54-51-15, 54-51-16, 54-51-17, 54-51-18, 54-51-19, 54-51-20, 54-51-21, 54-51-22, 54-51-23, 54-51-24, 54-51-30, 54-51-31, 54-51-32, 54-51-33, 54-51-40, 54-51-41, 54-51-42, 54-51-43, 54-51-44, 54-51-45, 54-51-47, 54-51-48, 54-51-49, 54-51-50-6, 54-51-50-7, 54-51-50-8, 54-51-50-9, 54-51-50-10, 54-51-50-11, 54-51-50-12, 54-51-50-13, 54-51-50-14, 54-51-50-15, 54-51-50-16, 54-51-50-19, 54-51-50-20, 54-51-50-21, 54-51-50-22, 54-51-50-23, 54-51-50-24, 54-51-50-30, 54-51-50-31, 54-51-50-32, 54-51-50-40, 54-51-50-41, 54-51-50-42, 54-51-50-43, 54-51-50-44, 54-51-50-45, 54-51-50-47, 54-51-50-48, 54-52-1, 54-52-2, 54-52-3, 54-52-6, 54-52-7, 54-52-8, 54-52-9, 54-52-10, 54-52-11, 54-52-12, 54-52-13, 54-52-14, 54-52-23, 54-52-24, 54-52-29, 54-52-30, 54-52-31, 54-52-32, 54-52-33, 54-52-34, 54-52-35, 54-52-36, 54-52-37, 54-52-41, 54-52-42, 54-52-43, 54-52-44, 54-52-45, 54-52-46, 54-52-47, 54-52-50-1, 54-52-50-2, 54-52-50-3, 54-52-50-6, 54-52-50-7, 54-52-50-8, 54-52-50-9, 54-52-50-10, 54-52-50-11, 54-52-50-12, 54-52-50-13, 54-52-50-14, 54-52-50-23, 54-52-50-24, 54-52-50-29, 54-52-50-30, 54-52-50-31, 54-52-50-34, 54-52-50-35, 54-52-50-36, 54-52-50-37, 54-52-50-41, 54-52-50-42, 54-52-50-43, 54-52-50-44, 54-52-50-45, 54-52-50-46, 54-52-50-47, 54-52-51-6, 54-52-51-7, 54-52-51-8, 54-52-51-9, 54-52-51-10, 54-52-51-11, 54-52-51-12, 54-52-51-13, 54-52-51-14, 54-52-51-23, 54-52-51-24, 54-52-51-30, 54-52-51-31, 54-52-51-41, 54-52-51-42, 54-52-51-43, 54-52-51-44, 54-52-51-45, 54-52-51-47, 54-52-51-50-6, 54-52-51-50-7, 54-52-51-50-8, 54-52-51-50-9, 54-52-51-50-10, 54-52-51-50-11, 54-52-51-50-12, 54-52-51-50-13, 54-52-51-50-14, 54-52-51-50-23, 54-52-51-50-24, 54-52-51-50-30, 54-52-51-50-31, 54-52-51-50-41, 54-52-51-50-42, 54-52-51-50-43, 54-52-51-50-44, 54-52-51-50-45, 54-52-51-50-47, 54-53-1, 54-53-2, 54-53-3, 54-53-4, 54-53-5, 54-53-6, 54-53-7, 54-53-8, 54-53-9, 54-53-10, 54-53-11, 54-53-12, 54-53-13, 54-53-14, 54-53-15,54-53-16, 54-53-17, 54-53-18, 54-53-19, 54-53-20, 54-53-21, 54-53-22, 54-53-23, 54-53-24, 54-53-25, 54-53-26, 54-53-27, 54-53-28, 54-53-29, 54-53-30, 54-53-31, 54-53-32, 54-53-33, 54-53-34, 54-53-35, 54-53-36, 54-53-37, 54-53-38, 54-53-39, 54-53-40, 54-53-41, 54-53-42, 54-53-43, 54-53-44, 54-53-45, 54-53-46, 54-53-47, 54-53-48, 54-53-49, 54-53-50-1, 54-53-50-2, 54-53-50-3, 54-53-50-6, 54-53-50-7, 54-53-50-8, 54-53-50-9, 54-53-50-10, 54-53-50-11, 54-53-50-12, 54-53-50-13, 54-53-50-14, 54-53-50-15, 54-53-50-16, 54-53-50-19, 54-53-50-20, 54-53-50-21, 54-53-50-22, 54-53-50-23, 54-53-50-24, 54-53-50-27, 54-53-50-28, 54-53-50-29, 54-53-50-30, 54-53-50-31, 54-53-50-32, 54-53-50-34, 54-53-50-35, 54-53-50-36, 54-53-50-37, 54-53-50-38, 54-53-50-40, 54-53-50-41, 54-53-50-42, 54-53-50-43, 54-53-50-44, 54-53-50-45, 54-53-50-46, 54-53-50-47, 54-53-50-48, 54-53-51-6, 54-53-51-7, 54-53-51-8, 54-53-51-9, 54-53-51-10, 54-53-51-11, 54-53-51-12, 54-53-51-13, 54-53-51-14, 54-53-51-15, 54-53-51-16, 54-53-51-17, 54-53-51-18, 54-53-51-19, 54-53-51-20, 54-53-51-21, 54-53-51-22, 54-53-51-23, 54-53-51-24, 54-53-51-30, 54-53-51-31, 54-53-51-32, 54-53-51-33, 54-53-51-40, 54-53-51-41, 54-53-51-42, 54-53-51-42, 54-53-51-44, 54-53-51-45, 54-53-51-47, 54-53-51-48, 54-53-51-49, 54-53-51-50-6, 54-53-51-50-7, 54-53-51-50-8, 54-53-51-50-9, 54-53-51-50-10, 54-53-51-50-11, 54-53-51-50-12, 54-53-51-50-13, 54-53-51-50-14, 54-53-51-50-15, 54-53-51-50-16, 54-53-51-50-19, 54-53-51-50-20, 54-53-51-50-21, 54-53-51-50-22, 54-53-51-50-23, 54-53-51-50-24, 54-53-51-50-30, 54-53-51-50-31, 54-53-51-50-32, 54-53-51-50-40, 54-53-51-50-41, 54-53-51-50-42, 54-53-51-50-43, 54-53-51-50-44, 54-53-51-50-45, 54-53-51-50-47, 54-53-51-50-48, 54-53-52-1, 54-53-52-2, 54-53-52-3, 54-53-52-6, 54-53-52-7, 54-53-52-8, 54-53-52-9, 54-53-52-10, 54-53-52-11, 54-53-52-12, 54-53-52-13, 54-53-52-14, 54-53-52-23, 54-53-52-24, 54-53-52-29, 54-53-52-30, 54-53-52-31, 54-53-52-32, 54-53-52-33, 54-53-52-34, 54-53-52-35, 54-53-52-36, 54-53-52-37, 54-53-52-41, 54-53-52-42, 54-53-52-43, 54-53-53-44, 54-53-52-45, 54-53-52-46, 54-53-52-47, 54-53-52-50-1, 54-53-52-50-2, 54-53-52-50-3, 54-53-52-50-6, 54-53-52-50-7, 54-53-52-50-8, 54-53-52-50-9, 54-53-52-50-10, 54-53-52-50-11, 54-53-52-50-12, 54-53-52-50-13, 54-53-52-50-14, 54-53-52-50-23, 54-53-52-50-24, 54-53-52-50-29, 54-53-52-50-30, 54-53-52-50-31, 54-53-52-50-34, 54-53-52-50-35, 54-53-52-50-36, 54-53-52-50-37, 54-53-52-50-41, 54-53-52-50-42, 54-53-52-50-43, 54-53-52-50-44, 54-53-52-50-45, 54-53-52-50-46, 54-53-52-50-47, 54-53-52-51-6, 54-53-52-51-7, 54-53-52-51-8, 54-53-52-51-9, 54-53-52-51-10, 54-53-52-51-11, 54-53-52-51-12, 54-53-52-51-13, 54-53-52-51-14, 54-53-52-51-23, 54-53-52-51-24, 54-53-52-51-30, 54-53-52-51-31, 54-53-52-51-41, 54-53-52-51-42, 54-53-52-51-43, 54-53-52-51-44, 54-53-52-51-45, 54-53-52-51-47, 54-53-52-51-50-6, 54-53-52-51-50-7, 54-53-52-51-50-8, 54-53-52-51-50-9, 54-53-52-51-50-10, 54-53-52-51-50-11, 54-53-52-51-50-12, 54-53-52-51-50-13, 54-53-52-51-50-14, 54-53-52-51-50-23, 54-53-52-51-50-24, 54-53-52-51-50-30, 54-53-52-51-50-31, 54-53-52-51-50-41, 54-53-52-51-50-42, 54-53-52-51-50-43, 54-53-52-51-50-44, 54-53-52-51-50-45 and 54-53-52-51-50-47. For each of these compound groups, designations 1.1.1.1 through 10.10.10.10 in Table B specifies a compound or genus of compounds as defined by the Table A substituents and any R⁸ moiety as described here or elsewhere herein.

Exemplary compounds in group 54-1 when R⁸ is —O— include compound 1.2.4.1, which is 11-oxa-3,7β-dihydroxy-16α-fluoro-17β-aminoandrost-1,3-diene, 1.1.5.9, which is 11-oxa-3,17β-dihydroxyandrost-1,3-diene, 1.1.6.9, which is 11-oxa-3,16α,17β-trihydroxyandrost-1,3-diene, 1.1.6.1, which is 11-oxa-3,16α-dihydroxy-17β-aminoandrost-1,3-diene and 1.1.4.9, which is 11-oxa-3,17β-dihydroxy-16α-fluoroandrost-1,3-diene. Exemplary compounds in group 54-7 when R⁸ is —O— include compound 1.2.4.1, which is 11-oxa-3β,7-dihydroxy-16α-fluoro-17β-aminoandrost-1,6-diene, 1.1.5.9, which is 11-oxa-3β,17β-dihydroxyandrost-1,6-diene, 1.1.6.9, which is 11-oxa-3β,16α,17β-trihydroxyandrost-1,6-diene, 1.1.6.1, which is 11-oxa-3β,16α-dihydroxy-17β-aminoandrost-1,6-diene and 1.1.4.9, which is 11-oxa-3β,17β-dihydroxy-16α-fluoroandrost-1,6-diene. Exemplary compounds in group 54-1 when R⁸ is —NH— include compound 1.2.4.1, which is 11-aza-3,7β-dihydroxy-16α-fluoro-17β-aminoandrost-1,3-diene, 1.1.5.9, which is 11-aza-3,17β-dihydroxyandrost-1,3-diene, 1.1.6.9, which is 11-aza-3,16α,17β-trihydroxyandrost-1,3-diene, 1.1.6.1, which is 11-aza-3,16α-dihydroxy-17β-aminoandrost-1,3-diene and 1.1.4.9, which is 11-aza-3,17β-dihydroxy-16α-fluoroandrost-1,3-diene. Exemplary compounds in group 54-1 when R⁸ is —S— include compound 1.2.4.1, which is 11-thia-3,7β-dihydroxy-16α-fluoro-17β-aminoandrost-1,3-diene, 1.1.5.9, which is 11-thia-3,17β-dihydroxyandrost-1,3-diene, 1.1.6.9, which is 11-thia-3,16α,17β-trihydroxyandrost-1,3-diene, 1.1.6.1, which is 11-thia-3,16α-dihydroxy-17β-aminoandrost-1,3-diene and 1.1.4.9, which is 11-thia-3,17β-dihydroxy-16α-fluoroandrost-1,3-diene. Exemplary compounds in group 54-53-1 when R⁸ and R⁹ are —O— include compound 1.2.4.1, which is 2,11-dioxa-3,7β-dihydroxy-16α-fluoro-17β-aminoandrost-1,3-diene, 1.1.5.9, which is 2,11-dioxa-3,17β-dihydroxyandrost-1,3-diene, 1.1.6.9, which is 2,11-dioxa-3,16α,17β-trihydroxyandrost-1,3-diene, 1.1.6.1, which is 2,11-dioxa-3,16α-dihydroxy-17β-aminoandrost-1,3-diene and 1.1.4.9, which is 2,11-dioxa-3,17β-dihydroxy-16α-fluoroandrost-1,3-diene. Exemplary compounds in group 54-44 when R⁸ is —CH(α-NH[CH₃])— include compound 1.2.4.1, which is 11α-methylamino-3β,7β-dihydroxy-16α-fluoro-17β-aminoandrost-5,7-diene, 1.1.5.9, which is 11α-methylamino-3β,17β-dihydroxyandrost-5,7-diene, 1.1.6.9, which is 11α-methylamino-3β,16α,17β-trihydroxyandrost-5,7-diene, 1.1.6.1, which is 11α-methylamino-3β,16α-dihydroxy-17β-aminoandrost-5,7-diene and 1.1.4.9, which is 11α-methylamino-3β,17β-dihydroxy-16α-fluoroandrost-5,7-diene. Exemplary compounds in group 54-2 when R⁸ is —CH(β-OH)— include compound 1.2.4.1, which is 11β,3,7β-trihydroxy-16α-fluoro-17β-amino-5β-androst-1,3-diene, 1.1.5.9, which is 11β,3,17β-trihydroxy-5β-androst-1,3-diene, 1.1.6.9, which is 11β,3,16α,17β-tetrahydroxy-5β-androst-1,3-diene, 1.1.6.1, which is 11β,3,16α-trihydroxy-17β-amino-5β-androst-1,3-diene and 1.1.4.9, which is 11β,3,17β-trihydroxy-16α-fluoro-5β-androst-1,3-diene. Exemplary compounds in group 54-3 when R⁸ is —CH(β-F)— include compound 1.2.4.1, which is 11β-fluoro-3,7β-dihydroxy-16α-fluoro-17β-aminoandrost-1,3,5-triene, 1.1.5.9, which is 11β-fluoro-3,17β-trihydroxyandrost-1,3,5-triene, 1.1.6.9, which is 11β-fluoro-3,16α,17β-tetrahydroxyandrost-1,3,5-triene, 1.1.6.1, which is 11β-fluoro-3,16α-trihydroxy-17β-aminoandrost-1,3,5-triene and 1.1.4.9, which is 11β-fluoro-3,17β-trihydroxy-16α-fluoroandrost-1,3,5-triene. Exemplary compounds in group 54-3 when R⁸ is —CH(β-C1-3 alkyl)- include compound 1.2.4.1, which is 11β-C1-3 alkyl-3,7β-dihydroxy-16α-fluoro-17β-aminoandrost-1,3,5-triene, 1.1.5.9, which is 11β-C1-3 alkyl-3,17β-trihydroxyandrost-1,3,5-triene, 1.1.6.9, which is 11β-C1-3 alkyl-3,16α,17β-tetrahydroxyandrost-1,3,5-triene, 1.1.6.1, which is 11β-C1-3 alkyl-3,16α-trihydroxy-17β-aminoandrost-1,3,5-triene and 1.1.4.9, which is 11β-C1-3 alkyl-3,17β-trihydroxy-16α-fluoroandrost-1,3,5-triene. Compounds or genera of compounds in the other group 54 compound groups where R⁸ is a moiety described here or elsewhere herein are defined as described in Tables A and B in the same manner.

Group 55. This group comprises compounds and compound genera in compound groups 1-54 described above, wherein R^{10G} is (1) a moiety other than hydrogen in the α-configuration or (2) hydrogen or another moiety as defined for this variable group in the β-configuration, instead of being in the α-configuration as shown in group 1. Exemplary R^{10G} moieties include —F, —Cl, —Br, —I, —OH, —H, ester, carbonate, C1-4 optionally substituted alkyl, C2-4 optionally substituted alkenyl or C2-4 optionally substituted alkynyl such as —CH₃, —C₂H₅, —CH₂OH, —CH₂F, —CHO, —CH═CH₂, —CH═CHOH, —C═CH, —C═C—CH₃ or another moiety described herein for R^{10G}, where any of these moieties is in the α-configuration or the β-configuration.

Groups of compounds in this group are defined essentially as described above, e.g., for groups 53 and 54. Compound groups in group 53 where R^{10G} is substituted or is in the β-configuration thus include 55-1, 55-2, 55-3, 55-4, 55-5, 55-6, 55-7, 55-8, 55-9, 55-10, 55-11, 55-12, 55-13, 55-14, 55-15, 55-16, 55-17, 55-18, 55-19, 55-20, 55-21, 51-22, 55-23, 55-24, 55-25, 55-26, 55-27, 55-28, 55-29, 55-30, 55-31, 55-32, 55-33, 55-34, 55-35, 55-36, 55-37, 55-38, 55-39, 55-40, 55-41, 55-42, 55-43, 55-44, 55-45, 55-46, 55-47, 55-48, 55-49, 55-50-1, 55-50-2, 55-50-3, 55-50-6, 55-50-7, 55-50-8, 55-50-9, 55-50-10, 55-50-11, 55-50-12, 55-50-13, 55-50-14, 55-50-15, 55-50-16, 55-50-19, 55-50-20, 55-50-21, 55-50-22, 55-50-23, 55-50-24, 55-50-27, 55-50-28, 55-50-29, 55-50-30, 55-50-31, 55-50-32, 55-50-34, 55-50-35, 55-50-36, 55-50-37, 55-50-38, 55-50-40, 55-50-41, 55-50-42, 55-50-43, 55-50-44, 55-50-45, 55-50-46, 55-50-47, 55-50-48, 55-51-6, 55-51-7, 55-51-8, 55-51-9, 55-51-10, 55-51-11, 55-51-12, 55-51-13, 55-51-14, 55-51-15, 55-51-16, 55-51-17, 55-51-18, 55-51-19, 55-51-20, 55-51-21, 55-51-22, 55-51-23, 55-51-24, 55-51-30, 55-51-31, 55-51-32, 55-51-33, 55-51-40, 55-51-41, 55-51-42, 55-51-43, 55-51-44, 55-51-45, 55-51-47, 55-51-48, 55-51-49, 55-51-50-6, 55-51-50-7, 55-51-50-8, 55-51-50-9, 55-51-50-10, 55-51-50-11, 55-51-50-12, 55-51-50-13, 55-51-50-14, 55-51-50-15, 55-51-50-16, 55-51-50-19, 55-51-50-20, 55-51-50-21, 55-51-50-22, 55-51-50-23, 55-51-50-24, 55-51-50-30, 55-51-50-31, 55-51-50-32, 55-51-50-40, 55-51-50-41, 55-51-50-42, 55-51-50-43, 55-51-50-44, 55-51-50-45, 55-51-50-48, 55-52-1, 55-52-2, 55-52-3, 55-52-6, 55-52-7, 55-52-8, 55-52-9, 55-52-10, 55-52-11, 55-52-12, 55-52-13, 55-52-14, 55-52-23, 55-52-24, 55-52-29, 55-52-30, 55-52-31, 55-52-32, 55-52-33, 55-52-34, 55-52-35, 55-52-36, 55-52-37, 55-52-41, 55-52-42, 55-52-43, 55-52-44, 55-52-45, 55-52-46, 55-52-47, 55-52-50-1, 55-52-50-2, 55-52-50-3, 55-52-50-6, 55-52-50-7, 55-52-50-8, 55-52-50-9, 55-52-50-10, 55-52-50-11, 55-52-50-12, 55-52-50-13, 55-52-50-14, 55-52-50-23, 55-52-50-24, 55-52-50-29, 55-52-50-30, 55-52-50-31, 55-52-50-34, 55-52-50-35, 55-52-50-36, 55-52-50-37, 55-52-50-41, 55-52-50-42, 55-52-50-43, 55-52-50-44, 55-52-50-45, 55-52-50-46, 55-52-50-47, 55-52-51-6, 55-52-51-7, 55-52-51-8, 55-52-51-9, 55-52-51-10, 55-52-51-11, 55-52-51-12, 55-52-51-13, 55-52-51-14, 55-52-51-23, 55-52-51-24, 55-52-51-30, 55-52-51-31, 55-52-51-41, 55-52-51-42, 55-52-51-43, 55-52-51-44, 55-52-51-45, 55-52-51-47, 55-52-51-50-6, 55-52-51-50-7, 55-52-51-50-8, 55-52-51-50-9, 55-52-51-50-10, 55-52-51-50-11, 55-52-51-50-12, 55-52-51-50-13, 55-52-51-50-14, 55-52-51-50-23, 55-52-51-50-24, 55-52-51-50-30, 55-52-51-50-31, 55-52-51-50-41, 55-52-51-50-42, 55-52-51-50-43, 55-52-51-50-44, 55-52-51-50-45, 55-52-51-50-47, 55-53-1, 55-53-2, 55-53-3, 55-53-4, 55-53-5, 55-53-6, 55-53-7, 55-53-8, 55-53-9, 55-53-10, 55-53-11, 55-53-12, 55-53-13, 55-53-14, 55-53-15, 55-53-16, 55-53-17, 55-53-18, 55-53-19, 55-53-20, 55-53-21, 55-53-22, 55-53-23, 55-53-24, 55-53-25, 55-53-26, 55-53-27, 55-53-28, 55-53-29, 55-53-30, 55-53-31, 55-53-32, 55-53-33, 55-53-34, 55-53-35, 55-53-36, 55-53-37, 55-53-38, 55-53-39, 55-53-40, 55-53-41, 55-53-42, 55-53-43, 55-53-44, 55-53-45, 55-53-46, 55-53-47, 55-53-48, 55-53-49, 55-53-50-1, 55-53-50-2, 55-53-50-3, 55-53-50-6, 55-53-50-7, 55-53-50-8, 55-53-50-9, 55-53-50-10, 55-53-50-11, 55-53-50-12, 55-53-50-13, 55-53-50-14, 55-53-50-15, 55-53-50-16, 55-53-50-19, 55-53-50-20, 55-53-50-21, 55-53-50-22, 55-53-50-23, 55-53-50-24, 55-53-50-27, 55-53-50-28, 55-53-50-29, 55-53-50-30, 55-53-50-31, 55-53-50-32, 55-53-50-34, 55-53-50-35, 55-53-50-36, 55-53-50-37, 55-53-50-38, 55-53-50-40, 55-53-50-41, 55-53-50-42, 55-53-50-43, 55-53-50-44, 55-53-50-45, 55-53-50-46, 55-53-50-47, 55-53-50-48, 55-53-51-6, 55-53-51-7, 55-53-51-8, 55-53-51-9, 55-53-51-10, 55-53-51-11, 55-53-51-12, 55-53-51-13, 55-53-51-14, 55-53-51-15, 55-53-51-16, 55-53-51-17, 55-53-51-18, 55-53-51-19, 55-53-51-20, 55-53-51-21, 55-53-51-22, 55-53-51-23, 55-53-51-24, 55-53-51-30, 55-53-51-31, 55-53-51-32, 55-53-51-33, 55-53-51-40, 55-53-51-41, 55-53-51-42, 55-53-51-43, 55-53-51-44, 55-53-51-45, 55-53-51-47, 55-53-51-48, 55-53-51-49, 55-53-51-50-6, 55-53-51-50-7, 55-53-51-50-8, 55-53-51-50-9, 55-53-51-50-10, 55-53-51-50-11, 55-53-51-50-12, 55-53-51-50-13, 55-53-51-50-14, 55-53-51-50-15, 55-53-51-50-16, 55-53-51-50-19, 55-53-51-50-20, 55-53-51-50-21, 55-53-51-50-22, 55-53-51-50-23, 55-53-51-50-24, 55-53-51-50-30, 55-53-51-50-31, 55-53-51-50-32, 55-53-51-50-40, 55-53-51-50-41, 55-53-51-50-42, 55-53-51-50-43, 55-53-51-50-44, 55-53-51-50-45, 55-53-51-50-47, 55-53-51-50-48, 55-53-52-1, 55-53-52-2, 55-53-52-3, 55-53-52-6, 55-53-52-7, 55-53-52-8, 55-53-52-9, 55-53-52-10, 55-53-52-11, 55-53-52-12, 55-53-52-13, 55-53-52-14, 55-53-52-23, 55-53-52-24, 55-53-52-29, 55-53-52-30, 55-53-52-31, 55-53-52-32, 55-53-53-33, 55-53-52-34, 55-53-52-35, 55-53-52-36, 55-53-52-37, 55-53-52-41, 55-53-52-42, 55-53-52-43, 55-53-52-44, 55-53-52-45, 55-53-52-46, 55-53-52-47, 55-53-52-50-1, 55-53-52-50-2, 55-53-52-50-3, 55-53-52-50-6, 55-53-52-50-7, 55-53-52-50-8, 55-53-52-50-9, 55-53-52-50-10, 55-53-52-50-11, 55-53-52-50-12, 55-53-52-50-13, 55-53-52-50-14, 55-53-52-50-23, 55-53-52-50-24, 55-53-52-50-29, 55-53-52-50-30, 55-53-52-50-31, 55-53-52-50-34, 55-53-52-50-35, 55-53-52-50-36, 55-53-52-50-37, 55-53-52-50-41, 55-53-52-50-42, 55-53-52-50-43, 55-53-52-50-44, 55-53-52-50-45, 55-53-52-50-46, 55-53-52-50-47, 55-53-52-51-6, 55-53-52-51-7, 55-53-52-51-8, 55-53-52-51-9, 55-53-52-51-10, 55-53-52-51-11, 55-53-52-51-12, 55-53-52-51-13, 55-53-52-51-14, 55-53-52-51-23, 55-53-52-51-24, 55-53-52-51-30, 55-53-52-51-31, 55-53-52-51-41, 55-53-52-51-42, 55-53-52-51-43, 55-53-52-51-44, 55-53-52-51-45, 55-53-52-51-47, 55-53-52-51-50-6, 55-53-52-51-50-7, 55-53-52-51-50-8, 55-53-52-51-50-9, 55-53-52-51-50-10, 55-53-52-51-50-11, 55-53-52-51-50-12, 55-53-52-51-50-13, 55-53-52-51-50-14, 55-53-52-51-50-23, 55-53-52-51-50-24, 55-53-52-51-50-30, 55-53-52-51-50-31, 55-53-52-51-50-41, 55-53-52-51-50-42, 55-53-52-51-50-43, 55-53-52-51-50-44, 55-53-52-51-50-45, 55-53-52-51-50-47, 55-54-1, 55-54-2, 55-54-3, 55-54-4, 55-54-5, 55-54-6, 55-54-7, 55-54-8, 55-54-9, 55-54-10, 55-54-11, 55-54-12, 55-54-13, 55-54-14, 55-54-15, 55-54-16, 55-54-17, 55-54-18, 55-54-19, 55-54-20, 55-54-21, 55-54-22, 55-54-23, 55-54-24, 55-54-25, 55-54-26, 55-54-27, 55-54-28, 55-54-29, 55-54-30, 55-54-31, 55-54-32, 55-54-33, 55-54-34, 55-54-35, 55-54-36, 55-54-37, 55-54-38, 55-54-39, 55-54-40, 55-54-41, 55-54-42, 55-54-43, 55-54-44, 55-54-45, 55-54-46, 55-54-47, 55-54-48, 55-54-49, 55-54-50-1, 55-54-50-2, 55-54-50-3, 55-54-50-6, 55-54-50-7, 55-54-50-8, 55-54-50-9, 55-54-50-10, 55-54-50-11, 55-54-50-12, 55-54-50-13, 55-54-50-14, 55-54-50-15, 55-54-50-16, 55-54-50-19, 55-54-50-20, 55-54-50-21, 55-54-50-22, 55-54-50-23, 55-54-50-24, 55-54-50-27, 55-54-50-28, 55-54-50-29, 55-54-50-30, 55-54-50-31, 55-54-50-32, 55-54-50-34, 55-54-50-35, 55-54-50-36, 55-54-50-37, 55-54-50-38, 55-54-50-40, 55-54-50-41, 55-54-50-42, 55-54-50-43, 55-54-50-44, 55-54-50-45, 55-54-50-46, 55-54-50-47, 55-54-50-48, 55-54-51-6, 55-54-51-7, 55-54-51-8, 55-54-51-9, 55-54-51-10, 55-54-51-11, 55-54-51-12, 55-54-51-13, 55-54-51-14, 55-54-51-15, 55-54-51-16, 55-54-51-17, 55-54-51-18, 55-54-51-19, 55-54-51-20, 55-54-51-21, 55-54-51-22, 55-54-51-23, 55-54-51-24, 55-54-51-30, 55-54-51-31, 55-54-51-32, 55-54-51-33, 55-54-51-40, 55-54-51-41, 55-54-51-42, 55-54-51-43, 55-54-51-44, 55-54-51-45, 55-54-51-47, 55-54-51-48, 55-54-51-49, 55-54-51-50-6, 55-54-51-50-7, 55-54-51-50-8, 55-54-51-50-9, 55-54-51-50-10, 55-54-51-50-11, 55-54-51-50-12, 55-54-51-50-13, 55-54-51-50-14, 55-54-51-50-15, 55-54-51-50-16, 55-54-51-50-19, 55-54-51-50-20, 55-54-51-50-21, 55-54-51-50-22, 55-54-51-50-23, 55-54-51-50-24, 55-54-51-50-30, 55-54-51-50-31, 55-54-51-50-32, 55-54-51-50-40, 55-54-51-50-41, 55-54-51-50-42, 55-54-51-50-43, 55-54-51-50-44, 55-54-51-50-45, 55-54-51-50-47, 55-54-51-50-48, 55-54-52-1, 55-54-52-2, 55-54-52-3, 55-54-52-6, 55-54-52-7, 55-54-52-8, 55-54-52-9, 55-54-52-10, 55-54-52-11, 55-54-52-12, 55-54-52-13, 55-54-52-14, 55-54-52-23, 55-54-52-24, 55-54-52-29, 55-54-52-30, 55-54-52-31, 55-54-52-32, 55-54-52-33, 55-54-52-34, 55-54-52-35, 55-54-52-36, 55-54-52-37, 55-54-52-41, 55-54-52-42, 55-54-52-43, 55-54-52-44, 55-54-52-45, 55-54-52-46, 55-54-52-47, 55-54-52-50-1, 55-54-52-50-2, 55-54-52-50-3, 55-54-52-50-6, 55-54-52-50-7, 55-54-52-50-8, 55-54-52-50-9, 55-54-52-50-10, 55-54-52-50-11, 55-54-52-50-12, 55-54-52-50-13, 55-54-52-50-14, 55-54-52-50-23, 55-54-52-50-24, 55-54-52-50-29, 55-54-52-50-30, 55-54-52-50-31, 55-54-52-50-34, 55-54-52-50-35, 55-54-52-50-36, 55-54-52-50-37, 55-54-52-50-41, 55-54-52-50-42, 55-54-52-50-43, 55-54-52-50-44, 55-54-52-50-45, 55-54-52-50-46, 55-54-52-50-47, 55-54-52-51-6, 55-54-52-51-7, 55-54-52-51-8, 55-54-52-51-9, 55-54-52-51-10, 55-54-52-51-11, 55-54-52-51-12, 55-54-52-51-13, 55-54-52-51-14, 55-54-52-51-23, 55-54-52-51-24, 55-54-52-51-30, 55-54-52-51-31, 55-54-52-51-41, 55-54-52-51-42, 55-54-52-51-43, 55-54-52-51-44, 55-54-52-51-45, 55-54-52-51-47, 55-54-52-51-50-6, 55-54-52-51-50-7, 55-54-52-51-50-8, 55-54-52-51-50-9, 55-54-52-51-50-10, 55-54-52-51-50-11, 55-54-52-51-50-12, 55-54-52-51-50-13, 55-54-52-51-50-14, 55-54-52-51-50-23, 55-54-52-51-50-24, 55-54-52-51-50-30, 55-54-52-51-50-31, 55-54-52-51-50-41, 55-54-52-51-50-42, 55-54-52-51-50-43, 55-54-52-51-50-44, 55-54-52-51-50-45, 55-54-52-51-50-47, 55-54-53-1, 55-54-53-2, 55-54-53-3, 55-54-53-4, 55-54-53-5, 55-54-53-6, 55-54-53-7, 55-54-53-8, 55-54-53-9, 55-54-53-10, 55-54-53-11, 55-54-53-12, 55-54-53-13, 55-54-53-14, 55-54-53-15, 55-54-53-16, 55-54-53-17, 55-54-53-18, 55-54-53-19, 55-54-53-20, 55-54-53-21, 55-54-53-22, 55-54-53-23, 55-54-53-24, 55-54-53-25, 55-54-53-26, 55-54-53-27, 55-54-53-28, 55-54-53-29, 55-54-53-30, 55-54-53-31, 55-54-53-32, 55-54-53-33, 55-54-53-34, 55-54-53-35, 55-54-53-36, 55-54-53-37, 55-54-53-38, 55-54-53-39, 55-54-53-40, 55-54-53-41, 55-54-53-42, 55-54-53-43, 55-54-53-44, 55-54-53-45, 55-54-53-46, 55-54-53-47, 55-54-53-48, 55-54-53-49, 55-54-53-50-1, 55-54-53-50-2, 55-54-53-50-3, 55-54-53-50-6, 55-54-53-50-7, 55-54-53-50-8, 55-54-53-50-9, 55-54-53-50-10, 55-54-53-50-11, 55-54-53-50-12, 55-54-53-50-13, 55-54-53-50-14, 55-54-53-50-15, 55-54-53-50-16, 55-54-53-50-19, 55-54-53-50-20, 50-54-53-50-21, 55-54-53-50-22, 55-54-53-50-23, 55-54-53-50-24, 55-54-53-50-27, 55-54-53-50-28, 55-54-53-50-29, 55-54-53-50-30, 55-54-53-50-31, 55-54-53-50-32, 55-54-53-50-34, 55-54-53-50-35, 55-54-53-50-36, 55-54-53-50-37, 55-54-53-50-38, 55-54-53-50-40, 55-54-53-50-41, 55-54-53-50-42, 55-54-53-50-43, 55-54-54-50-44, 55-54-53-50-45, 55-54-53-50-46, 55-54-53-50-47, 55-54-53-50-48, 55-54-53-51-6, 55-54-53-51-7, 55-54-53-51-8, 55-54-53-51-9, 55-54-53-51-10, 55-54-53-51-11, 55-54-53-51-12, 55-54-53-51-13, 55-54-53-51-14, 55-54-53-51-15, 55-54-53-51-16, 55-54-53-51-17, 55-54-53-51-18, 55-54-53-51-19, 55-54-53-51-20, 55-54-53-51-21, 55-54-53-51-22, 55-54-53-51-23, 55-54-53-51-24, 55-54-53-51-30, 55-54-53-51-31, 55-54-53-51-32, 55-54-53-51-33, 55-54-53-51-40, 55-54-53-51-41, 55-54-53-51-42, 55-54-53-51-43, 55-54-53-51-44, 55-54-53-51-45, 55-54-53-51-47, 55-54-53-51-48, 55-54-53-51-49, 55-54-53-51-50-6, 55-54-53-51-50-7, 55-54-53-51-50-8, 55-54-53-51-50-9, 55-54-53-51-50-10, 55-54-53-51-50-11, 55-54-53-51-50-12, 55-54-53-51-50-13, 55-54-53-51-50-14, 55-54-53-51-50-15, 55-54-53-51-50-16, 55-54-53-51-50-19, 55-54-53-51-50-20, 55-54-53-51-50-51, 55-54-53-51-50-22, 55-54-53-51-50-23, 55-54-53-51-50-24, 55-54-53-51-50-30, 55-54-53-51-50-31, 55-54-53-51-50-32, 55-54-53-51-50-40, 55-54-53-51-50-41, 55-54-53-51-50-42, 55-54-53-51-50-43, 55-54-53-51-50-44, 55-54-53-51-50-45, 55-54-53-51-50-47, 55-54-53-51-50-48, 55-54-53-52-1, 55-54-53-52-2, 55-54-53-52-3, 55-55-53-52-6, 55-54-53-53-7, 55-54-53-52-8, 55-54-53-52-9, 55-54-53-52-10, 55-54-53-52-11, 55-54-53-52-12, 55-54-53-52-13, 55-54-53-52-14, 55-54-53-52-23, 55-54-53-52-24, 55-54-53-52-29, 55-54-53-52-30, 55-54-53-52-31, 55-54-53-52-32, 55-54-53-52-33, 55-54-53-52-34, 55-54-53-52-35, 55-54-53-52-36, 55-54-53-52-37, 55-54-53-52-41, 55-54-53-52-42, 55-54-53-52-43, 55-54-53-52-44, 55-54-53-52-45, 55-54-53-52-46, 55-54-53-52-47, 55-54-53-52-50-1, 55-54-53-52-50-2, 55-54-53-52-50-3, 55-54-53-52-50-6, 55-54-53-52-50-7, 55-54-53-52-50-8, 55-54-53-52-50-9, 55-54-53-52-50-10, 55-54-53-52-50-11, 55-54-53-52-50-12, 55-54-53-52-50-13, 55-54-53-52-50-14, 55-54-53-52-50-23, 55-54-53-52-50-24, 55-54-53-52-50-29, 55-54-53-52-50-30, 55-54-53-52-50-31, 55-54-53-52-50-34, 55-54-53-52-50-35, 55-54-53-52-50-36, 55-54-53-52-50-37, 55-54-53-52-50-41, 55-54-53-52-50-42, 55-54-53-52-50-43, 55-54-53-52-50-44, 55-54-53-52-50-45, 55-54-53-52-50-46, 55-54-53-52-50-47, 55-54-53-52-51-6, 55-54-53-52-51-7, 55-54-53-52-51-8, 55-54-53-52-51-9, 55-54-53-52-51-10, 55-54-53-52-51-11, 55-54-53-52-51-12, 55-54-53-52-51-13, 55-54-53-52-51-14, 55-54-53-52-51-23, 55-54-53-52-51-24, 55-54-53-52-51-30, 55-54-53-52-51-31, 55-54-53-52-51-41, 55-54-53-52-51-42, 55-54-53-52-51-43, 55-54-53-52-51-44, 55-54-53-52-51-45, 55-54-53-52-51-47, 55-54-53-52-51-50-6, 55-54-53-52-51-50-7, 55-54-53-52-51-50-8, 55-54-53-52-51-50-9, 55-54-53-52-51-50-10, 55-54-53-52-51-50-11, 55-54-53-52-51-50-12, 55-54-53-52-51-50-13, 55-54-53-52-51-50-14, 55-54-53-52-51-50-23, 55-54-53-52-51-50-24, 55-54-53-52-51-50-30, 55-54-53-52-51-50-31, 55-54-53-52-51-50-41, 55-54-53-52-51-50-42, 55-54-53-52-51-50-43, 55-54-53-52-51-50-44, 55-54-53-52-51-50-45 and 55-54-53-52-51-50-47. For each of these compound groups, designations 1.1.1.1 through 10.10.10.10 in Table B specifies a compound or genus of compounds as defined by the Table A substituents and any R^{10G} moiety as described here or elsewhere herein.

Exemplary group 55-1 compounds where R^{10G} is fluorine in the α-configuration include 1.2.4.1, which is 3,70-dihydroxy-16α,9α-difluoro-17β-aminoandrost-1,3-diene, 1.1.6.9, which is 3,16α,17β-trihydroxy-9α-fluoroandrost-1,3-diene, 1.1.6.1, which is 3,16α-dihydroxy-9α-fluoro-17β-aminoandrost-1,3-diene and 1.1.4.9, which is 3,17β-dihydroxy-16α,9α-difluoroandrost-1,3-diene. Exemplary group 55-2 compounds where R^{10G} is fluorine in the α-configuration include 1.2.4.1, which is 3,7β-dihydroxy-16α,9α-difluoro-17β-amino-5β-androst-1,3-diene, 1.1.6.9, which is 3,16α,17β-trihydroxy-9α-fluoro-5β-androst-1,3-diene, 1.1.6.1, which is 3,16α-dihydroxy-9α-fluoro-17β-amino-5β-androst-1,3-diene and 1.1.4.9, which is 3,17β-dihydroxy-16α,9α-difluoro-5β-androst-1,3-diene. Exemplary group 55-3 compounds where R^{10G} is fluorine in the β-configuration include 1.2.4.1, which is 3,7β-dihydroxy-16α,9β-difluoro-17β-aminoandrost-1,3,5-triene, 1.1.6.9, which is 3,16α,17β-trihydroxy-9β-fluoroandrost-1,3,5-triene, 1.1.6.1, which is 3,16α-dihydroxy-9β-fluoro-17β-aminoandrost-1,3,5-triene and 1.1.4.9, which is 3,17β-dihydroxy-16α,9β-difluoroandrost-1,3,5-triene. Exemplary group 55-51-7 compounds where R^{10G} is fluorine in the α-configuration include 1.2.4.1, which is 3α,7-dihydroxy-16α,9α-difluoro-17β-aminoandrost-1,6-diene, 1.1.6.9, which is 3α,16α,17β-trihydroxy-9α-fluoroandrost-1,6-diene, 1.1.6.1, which is 3α,16α-dihydroxy-9α-fluoro-17β-aminoandrost-1,6-diene and 1.1.4.9, which is 3α,17β-dihydroxy-16α,9α-difluoroandrost-1,6-diene. Exemplary group 55-51-7 compounds where R^{10G} is chlorine in the α-configuration include 1.2.4.1, which is 3α,7-dihydroxy-9α-chloro-16α-fluoro-17β-aminoandrost-1,6-diene, 1.1.6.9, which is 3α,16α,17β-trihydroxy-9α-chloroandrost-1,6-diene, 1.1.6.1, which is 3α,16α-dihydroxy-9α-chloro-17β-aminoandrost-1,6-diene and 1.1.4.9, which is 3α,17β-dihydroxy-9α-chloro-16α-fluoroandrost-1,6-diene. Exemplary group 55-51-7 compounds where R^{10G} is fluorine in the β-configuration include 1.2.4.1, which is 3α,7-dihydroxy-16α,9β-difluoro-17β-aminoandrost-1,6-diene, 1.1.6.9, which is 3α,16α,17β-trihydroxy-9β-fluoroandrost-1,6-diene, 1.1.6.1, which is 3α,16α-dihydroxy-9β-fluoro-17β-aminoandrost-1,6-diene and 1.1.4.9, which is 3α,17β-dihydroxy-16α,9β-difluoroandrost-1,6-diene. Exemplary group 55-51-7 compounds where R^{10G} is hydroxyl in the α-configuration include 1.2.4.1, which is 3α,7,9α-trihydroxy-16α-fluoro-17β-aminoandrost-1,6-diene, 1.1.6.9, which is 3α,9α,16α,17β-tetrahydroxyandrost-1,6-diene, 1.1.6.1, which is 3α,16α-dihydroxy-9α-fluoro-17β-aminoandrost-1,6-diene and 1.1.4.9, which is 3α,9α,17β-trihydroxy-16α-fluoroandrost-1,6-diene. Compounds or genera of compounds in the other group 55 compound groups where R^{10G} is a moiety described here or elsewhere herein are defined as described in Tables A and B in the same manner. Exemplary R^{10G} moieties include C1-6 optionally substituted alkyl, —Cl, —Br, —I, —OH, —SH, —NH₂, —NHR^{PR}, ether, thioether, ester, thioester, C2-6 optionally substituted alkenyl and C2-6 optionally substituted alkynyl, which is in the α- or β-configuration.

Group 56. This group comprises compounds in the compound groups 1-55 described above, wherein (1) one, two, three or four of R^{10A}, R^{10B}, R^{10C} and R^{10D} is an independently selected moiety other than hydrogen and (2) each of R^{10A}, R^{10B}, R^{10C} and R^{10D} independently is in the α-configuration or the β-configuration when a double bond is not present at the steroid carbon atom to which it is bonded, i.e., there is no double bond at the 1-, 4- or 6-position. In this group, one or more of R^{10A}, R^{10B}, R^{10C} and R^{10D} is an independently selected moiety as defined herein, e.g., —H, halogen, hydroxyl, ketone, thiol, amino or optionally substituted alkyl. Other exemplary moieties include independently selected C1-4 optionally substituted alkyl, C1-4 optionally substituted alkenyl, C1-4 optionally substituted alkynyl, C1-4 optionally substituted alkoxy, optionally substituted monosaccharide, optionally substituted disaccharide, carbonate, carbamate, amide, amino acid and thioether. Exemplary moieties include independently selected —H, —²H, —³H, —F, —Cl, —Br, —I, —OH, ═O, —SH, ═S, —NH₂, ═NOH, ═NCH₃, ═NC₂H₅, ═NH, —CH₃, —C₂H₅, —CH₂OR^{X}, —OCH₃, —OC₂H₅, —OCH₂OR^{X}, —SCH₃, —SC₂H₅, —SCH₂OR^{X}, —OR^{X},—SR^{X}, —NHR^{X}, —N(R^{X})₂, —CH₂F, —CH═CH₂, —CH═CHOR^{X}, —C≡CH, —C≡CF, —C≡CCI, —C≡CBr, —C≡CI, —C≡COR^{X}, —C≡C—CH₃, —C≡CCH₂F, —C≡CCH₂Cl, —C≡CCH₂Br, —C≡CCH₂I, —C≡CCH₂OR^{X}, —C(O)CH₃, —C(O)CH₂R^{X}, —C(O)OCH₃, —C(O)CHOR^{X}, —C(O)OCH₃, ═CH₂, ═CHCH₃, ═CHCH₂OR^{X}, ═CHCH₂SR^{X}, ═CHCH₂NHR^{X}, ═CH₂CH₂OR^{X}, ═CH₂C(O)OR^{X}, —OCH₂OR^{X}, —OCH₂C(O)OR^{X}, —OCH₂CH₂C(O)OR^{X}, —OCH₂CH₂CH₂C(O)OR^{X}, —OCH₂NHR^{X}, —OCH₂CH₂NHR^{X}, —OCH₂CH₂CH₂NHR^{X}, —OC(O)CH₂NHR^{X}, —OC(O)CH₂CH₂NHR^{X}, —OC(O)CH₂CH₂CH₂NHR^{X}, —OCF₃, —OC₂H₄OR^{X}, —OC(O)CH₃, —OC(O)C₂H₅, —OC(O)C₂H₄OR^{X}, —OC(S)CH₃, —OC(S)C₂H₅, —SCH₂C(O)OR^{X}, —SCF₃, —SC₂H₄OR^{X}, —SC(O)CH₃, —SC(O)C₂H₅, —SC(O)C₂H₄OR^{X}, —NHCH₃, —N(CH₃)₂, —NHCH₂OR^{X}, —NHCH₂C(O)OR^{X}, —NHCF₃, —NHC₂H₅, —N(C₂H₅)₂, —N(C₃H₇)₂, —NHC₂H₄OR^{X}, —NHC(O)CH₃, —NHC(O)CF₃, —NHC(O)C₂H₅, —NHC(O)C₂H₄OR^{X}, —NHC(O)C₂H₄C(O)OR^{X}, —NHC(O)OCH₃, —NHC(O)OCF₃, —NHC(O)OC₂H₅, —NHC(O)OC₂H₄OR^{X}, —NHC(O)OC₂H₄C(O)OR^{X}, —NHCH₂C(O)OR^{X}, —NHCH(CH₃)C(O)OR^{X}, —O—S(O)(O)OR^{X}, —O—P(O)(O)OR^{X}, —S—P(O)(O)OR^{X} and —O—P(S)(O)OR^{X} moieties, where R^{X} independently are —H, a protecting group, optionally substituted alkyl or a counter ion for ionizable moieties, e.g., —CH₃, —C₂H₅, —C₃H₇, Na⁺, K⁺, chloride, bromide, iodide, methyl sulfonate, ethyl sulfonate, fumarate, lactate, succinate, amino, methylamine, diethylamine or another ion or another suitable salt or ion described herein.

As is apparent from the foregoing description, when no double bond is present at the carbon atoms at the 1-, 4-, 6- or 12-positions, R^{10A}, R^{10B}, R^{10C} and R^{10D} respectively can be in the α,α,α,α, α,α,α,β, α,α,β,α, α,β,α,α, β,α,α,α, α,α,β,β, α,β,α,β, β,α,α,β, α,β,β,α, β,α,β,α, β,β,α,α, α,β,β,β, β,α,β,β, β,β,α,β, β,β,β,α or β,β,β,β configurations. As used here, reference to, e.g., R^{10A}, R^{10B}, R^{10C} and R^{10D} respectively being in the α,β,α,β configurations means that R^{10A} is in the α-configuration, R^{10B} is in the β-configuration, R^{10C} is in the α-configuration and R^{10D} is in the β-configuration. Similarly, when R^{10A}, R^{10B}, R^{10C} and R^{10D} respectively are in the α,α,β,α configurations, R^{10A} is in the α-configuration, R^{10B} is in the α-configuration, R^{10C} is in the β-configuration and R^{10D} is in the α-configuration.

Thus, when a double bond is present at one or more of the 1-, 4- or 6-positions, the corresponding R^{10A}, R^{10B} or R^{10C} moiety will not be in a specified configuration. Group 56 contains compounds and genera of compounds in groups 1 through 55 above having structures where (1) a double bond is present at the 1-position, R^{10B}, R^{10C} and R^{10D} respectively are in the α,α,α, α,α,β, α,β,α, β,α,α, α,β,β, β,α,β, β,β,α or β,β,β configurations and R^{10A} is present at the 1-position with no specified configuration, (2) a double bond is present at the 4-position, R¹⁰, R^{10C} and R^{10D} respectively are in the α,α,α, α,α,β, α,β,α, β,α,α, α,β,β, β,α,β, β,β,α or β,β,β configurations and R^{10B} is present at the 4-position with no specified configuration, (3) a double bond is present at the 6-position, R^{10A}, R^{10B} and R^{10D} respectively are in the α,α,α, α,α,β, α,β,α, β,α,α, α, β, β, β,α,β, β,β,α or β,β,β configurations, and R^{10C} is present at the 6-position with no specified configuration, (4) a double bond is present at the 1-position and at the 4-position, R^{10C} and R^{10D} respectively are in the α,α, α,β, β,α, or β,β configurations and R^{10A} and R^{10B} are present at the 1- and 4-positions with no specified configuration, (5) a double bond is present at the 1-position and at the 6-position, R^{10B} and R^{10D} respectively are in the α,α, α,β, β,α, or β,β configurations and R^{10A} and R^{10C} are present at the 1- and 6-positions with no specified configuration, (6) a double bond is present at the 4-position and at the 6-position, R^{10A} and R^{10D} respectively are in the α,α, α,β, β,α, or β,β configurations and R^{10B} and R^{10C} are present at the 4- and 6-positions with no specified configuration, (7) a double bond is present at the 1-, 4- and 6-position, R^{10D} is in the α-configuration or the β-configuration, while R^{10A}, R^{10B} and R^{10C} are present at the 1-, 4- and 6-positions with no specified configuration and (8) one, two or more additional double bonds are optionally also present at the 8-, 9-, 11-, 14-, 15- or 16-positions for any compound or genus of compounds described in (1), (2), (3), (4), (5), (6) or (7).

Groups of compounds in this group are defined essentially as described above, e.g., for groups 53, 54.and 55. Exemplary compound groups with structures (1), (2), (3), (4), (5), (6), (7) or (8) described above include 56-1, 56-2, 56-3, 56-4, 56-5, 56-6, 56-7, 56-8, 56-9, 56-10, 56-11, 56-12, 56-13, 56-14, 56-15, 56-16, 56-17, 56-18, 56-19, 56-20, 56-21, 56-22, 56-23, 56-24, 56-25, 56-26, 56-27, 56-28, 56-29, 56-30, 56-31, 56-32, 56-33, 56-34, 56-35, 56-36, 56-37, 56-38, 56-39, 56-40, 56-41, 56-42, 56-43, 56-44, 56-45, 56-46, 56-47, 56-48, 56-49, 56-50-1, 56-50-2, 56-50-3, 56-50-6, 56-50-7, 56-50-8, 56-50-9, 56-50-10, 56-50-11, 56-50-12, 56-50-13, 56-50-14, 56-50-15, 56-50-16, 56-50-19, 56-50-20, 56-50-21, 56-50-22, 56-50-23, 56-50-24, 56-50-27, 56-50-28, 56-50-29, 56-50-30, 56-50-31, 56-50-32, 56-50-34, 56-50-35, 56-50-36, 56-50-37, 56-50-38, 56-50-40, 56-50-41, 56-50-42, 56-50-43, 56-50-44, 56-50-45, 56-50-46, 56-50-47, 56-50-48, 56-51-6, 56-51-7, 56-51-8, 56-51-9, 56-51-10, 56-51-11, 56-51-12, 56-51-13, 56-51-14, 56-51-15, 56-51-16, 56-51-17, 56-51-18, 56-51-19, 56-51-20, 56-51-21, 56-51-22, 56-51-23, 56-51-24, 56-51-30, 56-51-31, 56-51-32, 56-51-33, 56-51-40, 56-51-41, 56-51-42, 56-51-43, 56-51-44, 56-51-45, 56-51-47, 56-51-48, 56-51-49, 56-51-50-6, 56-51-50-7, 56-51-50-8, 56-51-50-9, 56-51-50-10, 56-51-50-11, 56-51-50-12, 56-51-50-13, 56-51-50-14, 56-51-50-15, 56-51-50-16, 56-51-50-19, 56-51-50-20, 56-51-50-21, 56-51-50-22, 56-51-50-23, 56-51-50-24, 56-51-50-30, 56-51-50-31, 56-51-50-32, 56-51-50-40, 56-51-50-41, 56-51-50-42, 56-51-50-43, 56-51-50-44, 56-51-50-45, 56-51-50-47, 56-51-50-48, 56-52-1, 56-52-2, 56-52-3, 56-52-6, 56-52-7, 56-52-8, 56-52-9, 56-52-10, 56-52-11, 56-52-12, 56-52-13, 56-52-14, 56-52-23, 56-52-24, 56-52-29, 56-52-30, 56-52-31, 56-52-32, 56-52-33, 56-52-34, 56-52-35, 56-52-36, 56-52-37, 56-52-41, 56-52-42, 56-52-43, 56-52-44, 56-52-45, 56-52-46, 56-52-47, 56-52-50-1, 56-52-50-2, 56-52-50-3, 56-52-50-6, 56-52-50-7, 56-52-50-8, 56-52-50-9, 56-52-50-10, 56-52-50-11, 56-52-50-12, 56-52-50-13, 56-52-50-14, 56-52-50-23, 56-52-50-24, 56-52-50-29, 56-52-50-30, 56-52-50-31, 56-52-50-34, 56-52-50-35, 56-52-50-36, 56-52-50-37, 56-52-50-41, 56-52-50-42, 56-52-50-43, 56-52-50-44, 56-52-50-45, 56-52-50-46, 56-52-50-47, 56-52-51-6, 56-52-51-7, 56-52-51-8, 56-52-51-9, 56-52-51-10, 56-52-51-11, 56-52-51-12, 56-52-51-13, 56-52-51-14, 56-52-51-23, 56-52-51-24, 56-52-51-30, 56-52-51-31, 56-52-51-41, 56-52-51-42, 56-52-51-43, 56-52-51-44, 56-52-51-45, 56-52-51-47, 56-52-51-50-6, 56-52-51-50-7, 56-52-51-50-8, 56-52-51-50-9, 56-52-51-50-10, 56-52-51-50-11, 56-52-51-50-12, 56-52-51-50-13, 56-52-51-50-14, 56-52-51-50-23, 56-52-51-50-24, 56-52-51-50-30, 56-52-51-50-31, 56-52-51-50-41, 56-52-51-50-42, 56-52-51-50-43, 56-52-51-50-44, 56-52-51-50-45, 56-52-51-50-47, 56-53-1, 56-53-2, 56-53-3, 56-53-4, 56-53-5, 56-53-6, 56-53-7, 56-52-8, 56-53-9, 56-53-10, 56-53-11, 56-53-12, 56-53-13, 56-53-14, 56-53-15, 56-52-16, 56-53-17, 56-53-18, 56-53-19, 56-53-20, 56-53-21, 56-53-22, 56-53-23, 56-53-24, 56-53-25, 56-53-26, 56-53-27, 56-53-28, 56-53-29, 56-53-30, 56-53-31, 56-53-32, 56-53-33, 56-53-34, 56-53-35, 56-53-36, 56-53-37, 56-53-38, 56-53-39, 56-53-40, 56-53-41, 56-53-42, 56-53-43, 56-53-44, 56-53-45, 56-53-46, 56-53-47, 56-53-48, 56-53-49, 56-52-50-1, 56-53-50-2, 56-53-50-3, 56-53-50-6, 56-53-50-7, 56-53-50-8, 56-53-50-9, 56-53-50-10, 56-53-50-11, 56-53-50-12, 56-53-50-13, 56-53-50-14, 56-53-50-15, 56-53-50-16, 56-53-50-19, 56-53-50-20, 56-53-50-21, 56-53-50-22, 56-53-50-23, 56-53-50-24, 56-53-50-27, 56-53-50-28, 56-53-50-29, 56-53-50-30, 56-53-50-31, 56-53-50-32, 56-53-50-34, 56-53-50-35, 56-53-50-36, 56-53-50-37, 56-53-50-38, 56-53-50-40, 56-53-50-41, 56-53-50-42, 56-53-50-43, 56-53-50-44, 56-53-50-45, 56-53-50-46, 56-53-50-47, 56-53-50-48, 56-53-51-50-6, 56-53-51-50-7, 56-53-51-50-8, 56-53-51-50-9, 56-53-51-50-10, 56-53-51-50-11, 56-53-51-50-12, 56-53-51-50-13, 56-53-51-50-14, 56-53-51-50-15, 56-53-51-50-16, 56-53-51-50-19, 56-53-51-50-20, 56-53-51-50-21, 56-53-51-50-22, 56-53-51-50-23, 56-53-51-50-24, 56-53-51-50-30, 56-53-51-50-31, 56-53-51-50-32, 56-53-51-50-40, 56-53-51-50-41, 56-53-51-50-42, 56-53-51-50-43, 56-53-51-50-44, 56-53-51-50-45, 56-53-51-50-47, 56-53-51-50-48, 53-51-6, 53-51-7, 53-51-8, 53-51-9, 53-51-10, 53-51-11, 53-51-12, 53-51-13, 53-51-14, 53-51-15, 53-51-16, 53-51-17, 53-51-18, 53-51-19, 53-51-20, 53-51-21, 53-51-22, 53-51-23, 53-51-24, 53-51-30, 53-51-31, 53-51-32, 53-51-33, 53-51-40, 53-51-41, 53-51-42, 53-51-43, 53-51-44, 53-51-45, 53-51-47, 53-52-48, 53-51-49, 56-53-52-1, 56-53-52-2, 56-53-52-3, 56-53-52-6, 56-53-52-7, 56-53-52-8, 56-53-52-9, 56-53-52-10, 56-53-52-11, 56-53-52-12, 56-53-52-13, 56-53-52-14, 56-53-52-23, 56-53-52-24, 56-53-52-29, 56-53-52-30, 56-53-52-31, 56-53-52-32, 56-53-52-33, 56-53-52-34, 56-53-52-35, 56-53-52-36, 56-53-52-37, 56-53-52-41, 56-53-52-42, 56-53-52-43, 56-53-52-44, 56-53-52-45, 56-53-52-46, 56-53-52-47, 56-53-52-50-1, 56-53-52-50-2, 56-53-52-50-3, 56-53-52-50-6, 56-53-52-50-7, 56-53-52-50-8, 56-53-52-50-9, 56-53-52-50-10, 56-53-52-50-11, 56-53-52-50-12, 56-53-52-50-13, 56-53-52-50-14, 56-53-52-50-23, 56-53-52-50-24, 56-53-52-50-29, 56-53-52-50-30, 56-53-52-50-31, 56-52-52-50-34, 56-53-52-50-35, 56-53-52-50-36, 56-53-52-50-37, 56-53-52-50-41, 56-53-52-50-42, 56-53-52-50-43, 56-53-52-50-44, 56-53-52-50-45, 56-53-52-50-46, 56-53-52-50-47, 56-53-52-51-6, 56-53-52-51-7, 56-53-52-51-8, 56-53-52-51-9, 56-53-52-51-10, 56-53-52-51-11, 56-53-52-51-12, 56-53-52-51-13, 56-53-52-51-14, 56-53-52-51-23, 56-53-52-51-24, 56-53-52-51-30, 56-53-52-51-31, 56-53-52-51-41, 56-53-52-51-42, 56-53-52-51-43, 56-53-52-51-44, 56-53-52-51-45, 56-53-52-51-47, 56-53-52-51-50-6, 56-53-52-51-50-7, 56-53-52-51-50-8, 56-53-52-51-50-9, 56-53-52-51-50-10, 56-53-52-51-50-11, 56-53-52-51-50-12, 56-53-52-51-50-13, 56-53-52-51-50-14, 56-53-52-51-50-23, 56-53-52-51-50-24, 56-53-52-51-50-30, 56-53-52-51-50-31, 56-53-52-51-50-41, 56-53-52-51-50-42, 56-53-52-51-50-43, 56-53-52-51-50-44, 56-53-52-51-50-45, 56-53-52-51-50-47, 56-54-1, 56-54-2, 56-54-3, 56-54-4, 56-54-5, 56-54-6, 56-54-7, 56-54-8, 56-54-9, 56-54-10, 56-11, 56-54-12, 56-54-13, 56-54-14, 56-54-15, 56-54-16, 56-54-17, 56-54-18, 56-54-19, 56-54-20, 56-54-21, 56-54-22, 56-54-23, 56-54-24, 56-54-25, 56-54-26, 56-54-27, 56-54-28, 56-54-29, 56-54-30, 56-54-31, 56-54-32, 56-54-33, 56-54-34, 56-54-35, 56-54-36, 56-54-37, 56-54-38, 56-54-39, 56-54-40, 56-54-41, 56-54-42, 56-54-43, 56-54-44-56-54-45, 56-54-46, 56-54-47, 56-54-48, 56-54-49, 56-54-50-1, 56-54-50-2, 56-54-50-3, 56-54-50-6, 56-54-50-7, 56-54-50-8, 56-54-50-9, 56-54-50-10, 56-54-50-11, 56-54-50-12, 56-54-50-13, 56-54-50-14, 56-54-50-15, 56-54-50-16, 56-54-50-19, 56-54-50-20, 56-54-50-21, 56-54-50-22, 56-54-50-23, 56-54-50-24, 56-54-50-27, 56-54-50-28, 56-54-50-29, 56-54-50-30, 56-54-50-31, 56-54-50-32, 56-54-50-34, 56-54-50-35, 56-54-50-36, 56-54-50-37, 56-54-50-38, 56-54-50-40, 56-54-50-41, 56-54-50-42, 56-54-50-43, 56-54-50-44, 56-54-50-45, 56-54-50-46, 56-54-50-47, 56-54-50-48, 56-54-51-6, 56-54-51-7, 56-54-51-8, 56-54-51-9, 56-54-51-10, 56-54-51-11, 56-54-51-12, 56-54-51-13, 56-54-51-14, 56-54-51-15, 56-54-51-16, 56-54-51-17, 56-54-51-18, 56-54-51-19, 56-54-51-20, 56-54-50-21, 56-54-51-22, 56-54-51-23, 56-54-51-24, 56-54-51-30, 56-54-51-31, 56-54-51-32, 56-54-51-33, 56-54-51-40, 56-54-51-41, 56-54-51-42, 56-54-51-43, 56-54-51-44, 56-54-51-45, 56-54-51-47, 56-54-51-48, 56-54-51-49, 56-54-51-50-6, 56-54-51-50-7, 56-54-51-50-8, 56-54-51-50-9, 56-54-51-50-10, 56-54-51-50-11, 56-54-51-50-12, 56-54-51-50-13, 56-54-51-50-14, 56-54-51-50-15, 56-54-51-50-16, 56-54-51-50-19, 56-54-51-50-20, 56-54-51-50-21, 56-54-51-50-22, 56-54-51-50-23, 56-54-51-50-24, 56-54-51-50-50, 56-54-51-50-31, 56-54-51-50-32, 56-54-51-50-40, 56-54-51-50-41, 56-54-51-50-42, 56-54-51-50-43, 56-54-51-50-44, 56-54-51-50-45, 56-54-51-50-47, 56-54-51-50-48, 56-54-52-1, 56-54-52-2, 56-54-52-3, 56-54-52-6, 56-54-52-7, 56-54-52-8, 56-54-52-9, 56-54-52-10, 56-54-52-11, 56-54-52-12, 56-54-52-13, 56-54-52-14, 56-54-52-23, 56-54-52-24, 56-54-52-29, 56-54-52-30, 56-54-52-31, 56-54-52-32, 56-54-52-33, 56-54-52-34, 56-54-52-35, 56-54-52-36, 56-54-52-37, 56-54-52-41, 56-54-52-42, 56-54-52-43, 56-54-42-44, 56-56-54-52-45, 56-54-52-46, 56-54-52-47, 56-54-52-50-1, 56-54-52-50-2, 56-54-52-50-3, 56-54-52-50-6, 56-54-52-50-7, 56-54-52-50-8, 56-54-52-50-9, 56-54-52-50-10, 56-54-52-50-11, 56-54-52-50-12, 56-54-52-50-13, 56-54-52-50-14, 56-54-52-50-23, 56-54-52-50-24, 56-54-52-50-29, 56-54-52-50-30, 56-54-52-50-31, 56-54-52-50-34, 56-54-52-50-35, 56-54-52-50-36, 56-54-52-50-37, 56-54-52-50-41, 56-54-52-50-42, 56-54-52-50-43, 56-54-52-50-44, 56-54-52-50-45, 56-54-52-50-46, 56-54-52-50-47, 56-54-52-51-6, 56-54-52-51-7, 56-54-52-51-8, 56-54-52-51-9, 56-54-52-51-10, 56-54-52-51-11, 56-54-52-51-12, 56-54-52-51-13, 56-54-52-51-14, 56-54-52-51-23, 56-54-52-51-24, 56-54-52-51-30, 56-54-52-51-31, 56-54-52-51-41, 56-54-52-51-42, 56-54-52-51-43, 56-54-52-51-44, 56-54-52-51-45, 56-54-52-51-47, 56-54-52-51-50-6, 56-54-52-51-50-7, 56-54-52-51-50-8, 56-54-52-51-50-9, 56-54-52-51-50-10, 56-54-52-5.1-50-11, 56-54-52-51-50-12, 56-54-52-51-50-13, 56-54-52-51-50-14, 56-54-52-51-50-23, 56-54-52-51-50-24, 56-54-52-51-50-30, 56-54-52-51-50-31, 56-54-52-51-50-41, 56-54-52-51-50-42, 56-54-52-51-50-43, 56-54-52-51-50-44, 56-54-52-51-50-45, 56-54-52-51-50-47, 56-54-53-1, 56-54-53-2, 56-54-53-3, 56-54-53-4, 56-54-53-5, 56-54-53-6, 56-54-53-7, 56-54-53-8, 56-54-53-9, 56-54-53-10, 56-54-53-11, 56-54-53-12, 56-54-53-13, 56-54-53-14, 56-54-53-15, 56-54-53-16, 56-54-53-17, 56-54-53-18, 56-54-53-19, 56-54-53-20, 56-54-53-21, 56-54-53-22, 56-54-53-23, 56-54-53-24, 56-54-53-25, 56-54-53-26, 56-54-53-27, 56-54-53-28, 56-54-53-29, 56-54-53-30, 56-54-53-31, 56-54-53-32, 56-54-53-33, 56-54-53-34, 56-54-53-35, 56-54-53-36, 56-54-53-37, 56-54-53-38, 56-54-53-39, 56-54-53-40, 56-54-53-41, 56-54-43-42, 56-54-53-43, 56-54-53-44, 56-54-53-45, 56-54-53-46, 56-54-53-47, 56-54-53-48, 56-54-53-49, 56-54-53-50-1, 56-54-53-50-2, 56-54-53-50-3, 56-54-53-50-6, 56-54-53-50-7, 56-54-53-50-8, 56-54-53-50-9, 56-54-53-50-10, 56-54-53-50-11, 56-54-53-50-12, 56-54-53-50-13, 56-54-53-50-14, 56-54-53-50-15, 56-54-53-50-16, 56-54-53-50-19, 56-54-53-50-20, 56-54-53-50-21, 56-54-53-50-22, 56-54-53-50-23, 56-54-53-50-24, 56-54-53-50-27, 56-54-53-50-28, 56-54-53-50-29, 56-54-53-50-30, 56-54-53-50-31, 56-54-53-50-32, 56-54-53-50-34, 56-54-53-50-35, 56-54-53-50-36, 56-54-53-50-37, 56-54-53-50-38, 56-54-53-50-40, 56-54-53-50-41, 56-54-53-50-42, 56-54-53-50-43, 56-54-53-50-44, 56-54-53-50-45, 56-54-53-50-46, 56-54-53-50-47, 56-54-53-50-48, 56-54-53-51-6, 56-54-53-51-7, 56-54-53-51-8, 56-54-53-51-9, 56-54-53-51-10, 56-54-53-51-11, 56-54-53-51-12, 56-54-53-51-13, 56-54-53-51-14, 56-54-53-51-15, 56-54-53-51-16, 56-54-53-51-17, 56-54-53-51-18, 56-54-53-51-19, 56-54-53-51-20, 56-54-53-51-21, 56-54-53-51-22, 56-54-53-51-23, 56-54-53-51-24, 56-54-53-51-30, 56-54-53-51-31, 56-54-53-51-32, 56-54-53-51-33, 56-54-53-51-40, 56-54-53-51-41, 56-54-53-51-42, 56-54-53-51-43, 56-54-52-51-44, 56-54-53-51-45, 56-54-53-51-47, 56-54-53-51-48, 56-54-53-51-49, 56-54-53-51-50-6, 56-54-53-51-50-7, 56-54-53-51-50-8, 56-54-53-51-50-9, 56-54-53-51-50-10, 56-54-53-51-50-11, 56-54-53-51-50-12, 56-54-53-51-50-13, 56-54-53-51-50-14, 56-54-53-51-50-15, 56-54-53-51-50-16, 56-54-53-51-50-19, 56-54-53-51-50-20, 56-54-53-51-50-21, 56-54-53-51-50-22, 56-54-53-51-50-23, 56-54-53-51-50-24, 56-54-53-51-50-30, 56-54-53-51-50-31, 56-54-53-51-50-32, 56-54-53-51-50-40, 56-54-53-51-50-41, 56-54-53-51-50-43, 56-54-53-51-50-43, 56-54-53-51-50-44, 56-54-53-51-50-45, 56-54-53-51-50-47, 56-54-53-51-50-48, 56-54-53-52-1, 56-54-53-52-2, 56-54-53-52-3, 56-54-53-52-6, 56-54-53-52-7, 56-54-53-52-8, 56-54-53-52-9, 56-54-53-52-10, 56-54-53-52-11, 56-54-53-52-12, 56-54-53-52-13, 56-54-53-52-14, 56-54-53-52-23, 56-54-53-52-24, 56-54-53-52-29, 56-54-53-53-30, 56-54-53-52-31, 56-54-53-52-32, 56-54-53-52-33, 56-54-53-52-34, 56-54-53-52-35, 56-54-53-52-36, 56-54-53-52-37, 56-54-53-52-41, 56-54-53-52-42, 56-54-53-52-43, 56-54-53-52-44, 56-54-53-52-45, 56-54-53-52-46, 56-54-53-52-47, 56-54-53-53-50-1, 56-54-53-52-50-2, 56-54-53-52-50-3, 56-54-53-52-50-6, 56-54-53-52-50-7, 56-54-53-53-50-8, 56-54-53-52-50-9, 56-54-53-52-50-10, 56-54-53-52-50-11, 56-54-53-52-50-12, 56-54-53-52-50-13, 56-54-53-52-50-14, 56-54-53-52-50-23, 56-54-53-52-50-24, 56-54-53-52-50-29, 56-54-53-52-50-30, 56-54-53-52-50-31, 56-54-53-52-50-34, 56-54-53-52-50-35, 56-54-53-52-50-36, 56-54-53-52-50-37, 56-54-53-52-50-41, 56-54-53-52-50-42, 56-54-53-52-50-43, 56-54-53-52-50-44, 56-54-53-52-50-45, 56-54-53-52-50-46, 56-54-53-52-50-47, 56-54-53-52-51-6, 56-54-53-52-51-7, 56-54-53-52-51-8, 56-54-53-52-51-9, 56-54-53-52-51-10, 56-54-53-52-51-11, 56-54-53-52-51-12, 56-54-53-52-51-13, 56-54-53-52-51-14, 56-54-53-52-51-23, 56-54-53-52-51-24, 56-54-53-52-51-30, 56-54-53-52-51-31, 56-54-53-52-51-41, 56-54-53-52-51-42, 56-54-53-52-51-43, 56-54-53-52-51-44, 56-54-53-52-51-45, 56-54-53-52-51-47, 56-54-53-52-51-50-6, 56-54-53-52-51-50-7, 56-54-53-52-51-50-8, 56-54-53-52-51-50-9, 56-54-53-52-51-50-10, 56-54-53-52-51-50-11, 56-54-53-52-51-50-12, 56-54-53-52-51-50-13, 56-54-53-52-51-50-14, 56-54-53-52-51-50-23, 56-54-53-52-51-50-24, 56-54-53-52-51-50-30, 56-54-53-52-51-50-31, 56-54-53-52-51-50-41, 56-54-53-52-51-50-42, 56-54-53-52-51-50-43, 56-54-53-52-51-50-44, 56-54-53-52-51-50-45, 56-54-53-52-51-50-47, 56-55-1, 56-55-2, 56-55-3, 56-55-4, 56-55-5, 56-55-6, 56-55-7, 56-55-8, 56-55-9, 56-55-10, 56-55-11, 56-55-12, 55-56-13, 56-55-14, 56-55-15, 56-55-16, 56-55-17, 56-55-18, 56-55-19, 56-55-20, 56-55-21, 56-55-22, 56-55-23, 56-55-24, 56-55-25, 56-55-26, 56-55-27, 56-55-28, 56-55-29, 56-55-30, 56-55-31, 56-55-32, 56-55-33, 56-55-34, 56-55-35, 56-55-36, 56-55-37, 56-55-38, 56-55-39, 56-55-40, 56-55-41, 56-55-42, 56-55-43, 56-55-44, 56-55-45, 56-55-46, 56-55-47, 56-55-48, 56-55-49, 56-55-50-1, 56-55-50-2, 56-55-50-3, 56-55-50-6, 56-55-50-7, 56-55-50-8, 56-55-50-9, 56-55-50-10, 56-55-50-11, 56-55-50-12, 56-55-50-13, 56-55-50-14, 56-55-50-15, 56-55-50-16, 56-55-50-19, 56-55-50-20, 56-55-50-21, 56-55-50-22, 56-55-50-23, 56-55-50-24, 56-55-50-27, 56-55-50-28, 56-55-50-29, 56-55-50-30, 56-55-50-31, 56-55-50-32, 56-55-50-34, 56-55-50-35, 56-55-50-36, 56-55-50-37, 56-55-50-38, 56-55-50-40, 56-55-50-41, 56-55-50-42, 56-55-50-43, 56-55-50-44, 56-55-50-45, 56-55-50-46, 56-55-50-47, 56-55-50-48, 56-55-51-6, 56-55-51-7, 56-55-51-8, 56-55-51-9, 56-55-51-10, 56-55-51-11, 56-55-51-12, 56-55-51-13, 56-55-51-14, 56-55-51-15, 56-55-51-16, 56-55-51-17, 56-55-51-18, 56-55-51-19, 56-55-51-20, 56-55-51-21, 56-55-51-22, 56-55-51-23, 56-55-51-24, 56-55-51-30, 56-55-51-31, 56-55-51-32, 56-55-51-33, 56-55-51-40, 56-55-51-41, 56-55-51-42, 56-55-51-43, 56-55-51-44, 56-55-51-45, 56-55-51-47, 56-55-51-48, 56-55-51-49, 56-55-51-50-6, 56-55-51-50-7, 56-55-51-50-8, 55-55-51-50-9, 56-55-51-50-10, 56-55-51-50-11, 56-55-51-50-12, 56-55-51-50-13, 56-55-51-50-14, 56-55-51-50-15, 56-55-51-50-16, 56-55-51-50-19, 56-55-51-50-20, 56-55-51-50-21, 56-55-51-50-22, 56-55-51-50-23, 56-55-51-50-24, 56-55-51-50-30, 56-55-51-50-31, 56-55-51-50-32, 56-55-51-50-40, 56-55-51-50-41, 56-55-51-50-42, 56-55-51-50-43, 56-55-51-50-44, 56-55-51-50-45, 56-55-51-50-47, 56-55-51-50-48, 56-55-52-1, 56-55-52-2, 56-55-52-3, 56-55-52-6, 56-55-52-7, 56-55-52-8, 56-55-52-9, 56-55-52-10, 56-55-52-11, 56-55-52-12, 56-55-52-13, 56-55-52-14, 56-55-52-23, 56-55-52-24, 56-55-52-29, 56-55-52-30, 56-55-52-31, 56-55-52-32, 56-55-52-33, 56-55-52-34, 56-55-52-35, 56-55-52-36, 56-5-52-37, 56-55-52-41, 56-55-52-42, 56-55-52-43, 56-55-52-44, 56-55-52-45, 56-55-52-46, 56-55-52-47, 56-55-52-50-1, 56-55-52-50-2, 56-55-52-50-3, 56-55-52-50-6, 56-55-53-50-7, 56-55-52-50-8, 56-55-52-50-9, 56-55-52-50-10, 56-55-52-50-11, 56-55-52-50-12, 56-55-52-50-13, 56-55-52-50-14, 56-55-52-50-23, 56-55-52-50-24, 56-55-52-50-29, 56-55-52-50-30, 56-55-52-50-31, 56-55-52-50-34, 56-55-52-50-35, 56-55-52-50-36, 56-55-52-50-37, 56-55-52-50-41, 56-55-52-50-42, 56-55-52-50-43, 56-55-52-50-44, 56-55-52-50-45, 56-55-52-50-46, 56-55-52-50-47, 56-55-52-51-6, 56-55-52-51-7, 56-55-52-51-8, 56-55-52-51-9, 56-55-52-51-10, 56-55-52-51-11, 56-55-52-51-12, 56-55-52-51-13, 56-55-52-51-14, 56-55-52-51-23, 56-55-52-51-24, 56-55-52-51-30, 56-55-52-51-31, 56-55-52-51-41, 56-55-52-51-42, 56-55-52-51-43, 56-55-52-51-44, 56-55-52-51-45, 56-55-52-51-47, 56-55-52-51-50-6, 56-55-52-51-50-7, 56-55-52-51-50-8, 56-55-52-51-50-9, 56-55-52-51-50-10, 56-55-52-51-50-11, 56-55-52-51-50-12, 56-55-52-51-50-13, 56-55-52-51-50-14, 56-55-52-51-50-23, 56-55-52-51-50-24, 56-55-52-51-50-30, 56-55-52-51-50-31, 56-55-52-51-50-41, 56-55-52-51-50-42, 56-55-52-51-50-43, 56-55-52-51-50-44, 56-55-52-51-50-45, 56-55-52-51-50-47, 56-55-53-1, 56-55-53-2, 56-55-53-3, 56-55-53-4, 56-55-53-5, 56-55-53-6, 56-55-53-7, 56-55-53-8, 56-55-53-9, 56-55-53-10, 56-55-53-11, 56-55-53-12, 56-55-53-13, 56-55-53-14, 56-55-53-15, 56-55-53-16, 56-55-53-17, 56-55-53-18, 56-55-53-19, 56-55-53-20, 56-55-53-21, 56-55-53-22, 56-55-53-23, 56-55-53-24, 56-55-53-25, 56-55-53-26, 56-55-53-27, 56-55-53-28, 56-55-53-29, 56-55-53-30, 56-55-53-31, 56-55-53-32, 56-55-53-33, 56-55-53-34, 56-55-53-35, 56-55-53-36, 56-55-53-37, 56-55-53-38, 56-55-53-39, 56-55-53-40, 56-55-53-41, 56-55-53-42, 56-55-53-43, 56-55-53-44, 56-55-53-45, 56-55-53-46, 56-55-53-47, 56-55-53-48, 56-55-53-49, 56-55-53-50-1, 56-55-53-50-2, 56-55-53-50-3, 56-55-53-50-6, 56-55-53-50-7, 56-55-53-50-8, 56-55-53-50-9, 56-55-53-50-10, 56-55-53-50-11, 56-55-53-50-12, 56-55-53-50-13, 56-55-53-50-14, 56-55-5-50-15, 56-55-53-50-16, 56-55-53-50-19, 56-55-53-50-20, 56-55-53-50-21, 56-55-53-50-22, 56-55-53-50-23, 56-55-53-50-24, 56-55-53-50-27, 56-55-53-50-28, 56-55-52-50-29, 56-55-53-50-30, 56-55-53-50-31, 56-55-53-50-32, 56-55-53-50-34, 56-55-53-50-35, 56-55-53-50-36, 56-55-53-50-37, 56-55-53-50-38, 56-55-53-50-40, 56-55-53-50-41, 56-55-53-50-42, 56-55-53-50-43, 56-55-53-50-44, 56-55-53-50-45, 56-55-53-50-46, 56-55-52-50-47, 56-55-53-50-48, 56-55-53-51-6, 56-55-53-51-7, 56-55-53-51-8, 56-55-53-51-9, 56-55-53-51-10, 56-55-53-51-11, 56-55-53-51-12, 56-55-53-51-13, 56-55-53-51-14, 56-55-53-51-15, 56-55-53-51-16, 56-55-53-51-17, 56-55-53-51-18, 56-55-53-51-19, 56-55-53-51-20, 56-55-53-51-21, 56-55-53-51-22, 56-55-53-51-23, 56-55-53-51-24, 56-55-53-51-30, 56-55-53-51-31, 56-55-53-51-32, 56-55-53-51-33, 56-55-53-51-40, 56-55-53-51-41, 56-55-53-51-42, 56-55-53-51-43, 56-55-53-51-44, 56-55-53-51-45, 56-55-53-51-47, 56-55-53-51-48, 56-55-53-51-49, 56-55-53-51-50-6, 56-55-53-51-50-7, 56-55-53-51-50-8, 56-55-53-51-50-9, 56-55-53-51-50-10, 56-55-53-51-50-11, 56-55-53-51-50-12, 56-55-53-51-50-13, 56-55-53-51-50-14, 56-55-53-51-50-15, 56-55-53-51-50-16, 56-55-53-51-50-19, 56-55-53-51-50-20, 56-55-53-51-50-21, 56-55-53-51-50-22, 56-55-53-51-50-23, 56-55-53-51-50-24, 56-55-53-51-50-30, 56-55-53-51-50-31, 56-55-53-51-50-32, 56-55-53-51-50-40, 56-55-53-51-50-41, 56-55-53-51-50-42, 56-55-53-51-50-43, 56-55-53-51-50-44, 56-55-53-51-50-45, 56-55-53-51-50-47, 56-55-53-51-50-48, 56-55-53-52-1, 56-55-53-52-2, 56-55-53-52-3, 56-55-53-52-6, 56-55-53-52-7, 56-55-53-52-8, 56-55-53-52-9, 56-55-53-52-10, 56-55-53-52-11, 56-55-53-52-12, 56-55-53-52-13, 56-55-53-52-14, 56-55-53-52-23, 56-55-53-52-24, 56-55-53-52-29, 56-55-53-52-30, 56-55-53-52-31, 56-55-53-52-32, 56-55-53-52-33, 56-55-53-52-34, 56-55-53-52-54, 56-55-53-52-36, 56-55-53-52-37, 56-55-53-52-41, 56-55-53-52-42, 56-55-53-52-43, 56-55-53-52-44, 56-55-53-52-45, 56-55-53-52-46, 56-55-53-52-47, 56-55-53-52-50-1, 56-55-53-52-50-2, 56-55-53-52-50-3, 56-55-53-52-50-6, 56-55-53-52-50-7, 56-55-53-52-50-8, 56-55-53-52-50-9, 56-55-53-52-50-10, 56-55-53-52-50-11, 56-55-53-52-50-12, 56-55-53-52-50-13, 56-55-53-52-50-14, 56-55-53-52-50-23, 56-55-53-52-50-24, 56-55-53-52-50-29, 56-55-53-52-50-30, 56-55-53-52-50-31, 56-55-53-52-50-34, 56-55-53-52-50-35, 56-55-53-52-50-36, 56-55-53-52-50-37, 56-55-53-52-50-41, 56-55-53-52-50-42, 56-55-53-52-50-43, 56-55-53-52-50-44, 56-55-53-52-50-45, 56-55-53-52-50-46, 56-55-53-52-50-47, 56-55-53-52-51-6, 56-55-53-52-51-7, 56-55-53-52-51-8, 56-55-53-52-51-9, 56-55-53-52-51-10, 56-55-53-52-51-11, 56-55-53-52-51-12, 56-55-53-52-51-13, 56-55-53-52-51-14, 56-55-53-52-51-23, 56-55-53-52-51-24, 56-55-53-52-51-30, 56-55-53-52-51-31, 56-55-53-52-51-41, 56-55-53-52-51-42, 56-55-53-52-51-43, 56-55-53-52-51-44, 56-55-53-52-51-45, 56-55-53-52-51-47, 56-55-53-52-51-50-6, 56-55-53-52-51-50-7, 56-55-53-52-51-50-8, 56-55-53-52-51-50-9, 56-55-53-52-51-50-10, 56-55-53-52-51-50-11, 56-55-53-52-51-50-12, 56-55-53-52-51-50-13, 56-55-53-52-51-50-14, 56-55-53-52-51-50-23, 56-55-53-52-51-50-24, 56-55-53-52-51-50-30, 56-55-53-52-51-50-31, 56-55-53-52-51-50-41, 56-55-53-52-51-50-42, 56-55-53-52-51-50-43, 56-55-53-52-51-50-44, 56-55-53-52-51-50-45, 56-55-53-52-51-50-47, 56-55-54-1, 56-55-54-2, 56-55-54-3, 56-55-54-4, 56-55-54-5, 56-55-54-6, 56-55-54-7, 56-55-54-8, 56-55-54-9, 56-55-54-10, 56-55-54-11, 56-55-54-12, 56-55-54-13, 56-55-54-14, 56-55-54-15, 56-55-54-16, 56-55-54-17, 56-55-54-18, 56-55-54-19, 56-55-54-20, 56-55-54-21, 56-55-54-22, 56-55-54-23, 56-55-54-24, 56-55-54-25, 56-55-54-26, 56-55-54-27, 56-55-54-28, 56-55-54-29, 56-55-54-30, 56-55-54-31, 56-55-54-32, 56-55-54-33, 56-55-54-34, 56-55-54-35, 56-55-54-36, 56-55-54-37, 56-55-54-38, 56-55-54-39, 56-55-54-40, 56-55-54-41, 56-55-54-42, 56-55-54-43, 56-55-54-44, 56-55-54-45, 56-55-54-46, 56-55-54-47, 56-55-54-48, 56-55-54-49, 56-55-54-50-1, 56-55-54-50-2, 56-55-54-50-3, 56-55-54-50-6, 56-55-54-50-7, 56-55-54-50-8, 56-55-54-50-9, 56-55-54-50-10, 56-55-54-50-11, 56-55-54-50-12, 56-55-54-50-13, 56-55-54-50-14, 56-55-54-50-15, 56-55-54-50-16, 56-55-54-50-19, 56-55-54-50-20, 56-55-54-50-21, 56-55-54-50-22, 56-55-54-50-23, 56-55-54-50-24, 56-55-54-50-27, 56-55-54-50-28, 56-55-54-50-29, 56-55-54-50-30, 56-55-54-50-31, 56-55-54-50-32, 56-55-54-50-34, 56-55-54-50-35, 56-55-54-50-36, 56-55-54-50-37, 56-55-54-50-38, 56-55-54-50-40, 56-55-54-50-41, 56-55-54-50-42, 56-55-54-50-43, 56-55-54-50-44, 56-55-54-50-45, 56-55-54-50-46, 56-55-54-50-47, 56-55-54-50-48, 56-55-54-51-6, 56-55-54-51-7, 56-55-54-51-8, 56-55-54-51-9, 56-55-54-51-10, 56-55-54-51-11, 56-55-54-51-12, 56-55-54-51-13, 56-55-54-51-14, 56-55-54-51-15, 56-55-54-51-16, 56-55-54-51-17, 56-55-54-51-18, 56-55-54-51-19, 56-55-54-51-20, 56-55-54-51-21, 56-55-54-51-22, 56-55-54-51-23, 56-55-54-51-24, 56-55-54-51-30, 56-55-54-51-31, 56-55-54-51-32, 56-55-54-51-33, 56-55-54-51-40, 56-55-54-51-41, 56-55-54-51-42, 56-55-54-51-43, 56-55-54-51-44, 56-55-54-51-45, 56-55-54-51-47, 56-55-54-51-48, 56-55-54-51-49, 56-55-54-51-50-6, 56-55-54-51-50-7, 56-55-54-51-50-8, 56-55-54-51-50-9, 56-55-54-51-50-10, 56-55-54-51-50-11, 56-55-54-51-50-12, 56-55-54-51-50-13,56-55-54-51-50-14, 56-55-54-51-50-15, 56-55-54-51-50-16, 56-55-54-51-50-19, 56-55-54-51-50-20, 56-55-54-51-50-21, 56-55-54-51-50-22, 56-55-54-51-50-23, 56-55-54-51-50-24, 56-55-54-51-50-30, 56-55-54-51-50-31, 56-55-54-51-50-32, 56-55-54-51-50-40, 56-55-54-51-50-41, 56-55-54-51-50-42, 56-55-54-51-50-43, 56-55-54-51-50-44, 56-55-54-51-50-45, 56-55-54-51-50-47, 56-55-54-51-50-48, 56-55-54-52-1, 56-55-54-52-2, 56-55-54-52-3, 56-55-54-52-6, 56-55-54-52-7, 56-55-54-52-8, 56-55-54-52-9, 56-55-54-52-10, 56-55-54-52-11, 56-55-54-52-12, 56-55-54-52-13, 56-55-54-52-14, 56-55-54-52-23, 56-55-54-52-24, 56-55-54-52-29, 56-55-54-52-30, 56-55-54-52-31, 56-55-54-52-32, 56-55-54-52-33, 56-55-54-52-34, 56-55-54-52-35, 56-55-54-52-36, 56-55-54-52-37, 56-55-54-52-41, 56-55-54-52-42, 56-55-54-52-43, 56-55-54-52-44, 56-55-54-52-45, 56-55-54-52-46, 56-55-54-52-47, 56-55-54-52-50-1, 56-55-54-52-50-2, 56-55-54-52-50-3, 56-55-54-52-50-6, 56-55-54-52-50-7, 56-55-54-52-50-8, 56-55-54-52-50-9, 56-55-54-52-50-10, 56-55-54-52-50-11, 56-55-54-52-50-12, 56-55-54-52-50-12, 56-55-54-52-50-14, 56-55-54-52-50-23, 56-55-54-52-50-24, 56-55-54-52-50-29, 56-55-54-52-50-30, 56-55-54-52-50-31, 56-55-54-52-50-34, 56-55-54-52-50-35, 56-55-54-52-50-36, 56-55-54-52-50-37, 56-55-54-52-50-41, 56-55-54-52-50-42, 56-55-54-52-50-43, 56-55-54-52-50-44, 56-55-54-52-50-45, 56-55-54-52-50-46, 56-55-54-52-50-47, 56-55-54-52-51-6, 56-55-54-52-51-7, 56-55-54-52-51-8, 56-55-54-52-51-9, 56-55-54-52-51-10, 56-55-54-52-51-11, 56-55-54-52-51-12, 56-55-54-52-51-13, 56-55-54-52-51-14, 56-55-54-52-51-23, 56-55-54-52-51-24, 56-55-54-52-51-30, 56-55-54-52-51-31, 56-55-54-52-51-41, 56-55-54-52-51-42, 56-55-54-52-51-43, 56-55-54-52-51-44, 56-55-54-52-51-45, 56-55-54-52-51-47, 56-55-54-52-51-50-6, 56-55-54-52-51-50-7, 56-55-54-52-51-50-8, 56-55-54-52-51-50-9, 56-55-54-52-51-50-10, 56-55-54-52-51-50-11, 56-55-54-52-51-50-12, 56-55-54-52-51-50-13, 56-55-54-52-51-50-14, 56-55-54-52-51-50-23, 56-55-54-52-51-50-24, 56-55-54-52-51-50-30, 56-55-54-52-51-50-31, 56-55-54-52-51-50-41, 56-55-54-52-51-50-42, 56-55-54-52-51-50-43, 56-55-54-52-51-50-44, 56-55-54-52-51-50-45, 56-55-54-52-51-50-47, 56-55-54-53-1, 56-55-54-53-2, 56-55-55-53-3, 56-55-54-53-4, 56-55-54-53-5, 56-55-54-53-6, 56-55-54-53-7, 56-55-54-53-8, 56-55-54-53-9, 56-55-54-53-10, 56-55-54-53-11, 56-55-54-53-12, 56-55-54-53-13, 56-55-54-53-14, 56-55-54-53-15, 56-55-54-53-16, 56-55-54-53-17, 56-55-54-53-18, 56-55-54-53-19, 56-55-54-53-20, 56-55-54-53-21, 56-55-54-53-22, 56-55-54-53-23, 56-55-54-53-24, 56-55-54-53-25, 56-55-54-53-26, 56-55-54-53-27, 56-55-54-53-28, 56-55-54-53-29, 56-55-54-53-30, 56-55-54-53-31, 56-55-54-53-32, 56-55-54-53-33, 56-55-54-53-34, 56-55-54-53-35, 56-55-54-53-36, 56-55-54-53-37, 56-55-54-53-38, 56-55-54-53-39, 56-55-54-53-40, 56-55-54-53-41, 56-55-54-53-42, 56-55-54-53-43, 56-55-54-53-44, 56-55-54-53-45, 56-55-54-53-46, 56-55-54-53-47, 56-55-54-53-48, 56-55-54-53-49, 56-55-54-53-50-1, 56-55-54-53-50-2, 56-55-54-53-50-3, 56-55-54-53-50-6, 56-55-54-53-50-7, 56-55-54-53-50-8, 56-55-54-53-50-9, 56-55-54-53-50-10, 56-55-54-53-50-11, 56-55-54-53-50-12, 56-55-54-53-50-13, 56-55-54-53-50-14, 56-55-54-53-50-15, 56-55-54-53-50-16, 56-55-54-53-50-19, 56-55-54-53-50-20, 56-55-54-53-50-21, 56-55-54-53-50-22, 56-55-54-53-50-23, 56-55-54-53-50-24, 56-55-54-53-50-27, 56-55-54-53-50-28, 56-55-54-53-50-29, 56-55-54-53-50-30, 56-55-54-53-50-31, 56-55-54-53-50-32, 56-55-54-53-50-34, 56-55-54-53-50-35, 56-55-54-53-50-36, 56-55-54-53-50-37, 56-55-54-53-50-38, 56-55-54-53-50-40, 56-55-54-53-50-41, 56-55-54-53-50-42, 56-55-54-53-50-43, 56-55-54-53-50-44, 56-55-54-53-50-45, 56-55-54-53-50-46, 56-55-54-53-50-47, 56-55-54-53-50-48, 56-55-54-53-51-6, 56-55-54-53-51-7, 56-55-54-53-51-8, 56-55-54-53-51-9, 56-55-54-53-51-10, 56-55-54-53-51-11, 56-55-54-53-51-12, 56-55-54-53-51-13, 56-55-54-53-51-14, 56-55-54-53-51-15, 56-55-54-53-51-16, 56-55-54-53-51-17, 56-55-54-53-51-18, 56-55-54-53-51-19, 56-55-54-53-51-20, 56-55-54-53-51-21, 56-55-54-53-51-22, 56-55-54-53-51-23, 56-55-54-53-51-24, 56-55-54-53-51-30, 56-55-54-53-51-31, 56-55-54-53-51-32, 56-55-54-53-51-33, 56-55-54-53-51-40, 56-55-54-53-51-41, 56-55-54-53-51-42, 56-55-54-53-51-50-43, 56-55-54-53-51-44, 56-55-54-53-51-45, 56-55-54-53-51-47, 56-55-54-53-51-48, 56-55-54-53-51-49, 56-55-54-53-51-50-6, 56-55-54-53-51-50-7, 56-55-54-53-51-50-8, 56-55-54-53-51-50-9, 56-55-54-53-51-50-10, 56-55-54-53-51-50-11, 56-55-54-53-51-50-12, 56-55-54-53-51-50-13, 56-55-54-53-51-50-14, 56-55-54-53-51-50-15, 56-55-54-53-51-50-16, 56-55-54-53-51-50-19, 56-55-54-53-51-50-20, 56-55-54-53-51-50-21, 56-55-54-53-51-50-22, 56-55-54-53-51-50-23, 56-55-54-53-51-50-24, 56-55-54-53-51-50-30, 56-55-54-53-51-50-31, 56-55-54-53-51-50-32, 56-55-54-53-51-50-40, 56-55-54-53-51-50-41, 56-55-54-53-51-50-42, 56-55-54-53-51-50-43, 56-55-54-53-51-50-44, 56-55-54-53-51-50-45, 56-55-54-53-51-50-47, 56-55-54-53-51-50-48, 56-55-54-53-52-1, 56-55-54-53-52-2, 56-55-54-53-52-3, 56-55-54-53-52-6, 56-55-54-53-52-7, 56-55-54-53-52-8, 56-55-54-53-52-9, 56-55-54-53-52-10, 56-55-54-53-52-11, 56-55-54-53-52-12, 56-55-54-53-52-13, 56-55-54-53-52-14, 56-55-54-53-52-23, 56-55-54-53-52-24, 56-55-54-53-52-29, 56-55-54-53-52-30, 56-55-54-53-52-31, 56-55-54-53-52-32, 56-55-54-53-52-33, 56-55-54-53-52-34, 56-55-54-53-52-35, 56-55-54-53-52-36, 56-55-54-53-52-37, 56-55-54-53-52-41, 56-55-54-53-52-42, 56-55-54-53-52-43, 56-55-54-53-52-44, 56-55-54-53-52-45, 56-55-54-53-52-46, 56-55-54-53-52-47, 56-55-54-53-52-50-1, 56-55-54-53-52-50-2, 56-55-54-53-52-50-3, 56-55-54-53-52-50-6, 56-55-54-53-52-50-7, 56-55-54-53-52-50-8, 56-55-54-53-52-50-9, 56-55-54-53-52-50-10, 56-55-54-53-52-50-11, 56-55-54-53-52-50-12, 56-55-54-53-52-50-13, 56-55-54-53-52-50-14, 56-55-54-53-52-50-23, 56-55-54-53-52-50-24, 56-55-54-53-52-50-29, 56-55-54-53-52-50-30, 56-55-54-53-52-50-31, 56-55-54-53-52-50-34, 56-55-54-53-52-50-35, 56-55-54-53-52-50-36, 56-55-54-53-52-50-37, 56-55-54-53-52-50-41, 56-55-54-53-52-50-42, 56-55-54-53-52-50-43, 56-55-54-53-52-50-44, 56-55-54-53-52-50-45, 56-55-54-53-52-50-46, 56-55-54-53-52-50-47, 56-55-54-53-52-51-6, 56-55-54-53-52-51-7, 56-55-54-53-52-51-8, 56-55-54-53-52-51-9, 56-55-54-53-52-51-10, 56-55-54-53-52-51-11, 56-55-54-53-52-51-12, 56-55-54-53-52-51-13, 56-55-54-53-52-51-14, 56-55-54-53-52-51-23, 56-55-54-53-52-51-24, 56-55-54-53-52-51-30, 56-55-54-53-52-51-31, 56-55-54-53-52-51-41, 56-55-54-53-52-51-42, 56-55-54-53-52-51-43, 56-55-54-53-52-51-44, 56-55-54-53-52-51-45, 56-55-54-53-52-51-47, 56-55-54-53-52-51-50-6, 56-55-54-53-52-51-50-7, 56-55-54-53-52-51-50-8, 56-55-54-53-52-51-50-9, 56-55-54-53-52-51-50-10, 56-55-54-53-52-51-50-11, 56-55-54-53-52-51-50-12, 56-55-54-53-52-51-50-13, 56-55-54-53-52-51-50-14, 56-55-54-53-52-51-50-23, 56-55-54-53-52-51-50-24, 56-55-54-53-52-51-50-30, 56-55-54-53-52-51-50-31, 56-55-55-54-53-52-51-50-41, 56-55-54-53-52-51-50-42, 56-55-54-53-52-51-50-43, 56-55-54-53-52-51-50-44, 56-55-54-53-52-51-50-45 and 56-55-54-53-52-51-50-47. For each of these compound groups, designations 1.1.1.1 through 10.10.10.10 in Table B specifies a compound or genus of compounds as defined by the Table A substituents and any independently selected R^{10A}, R^{10B}, R^{10C} and R^{10D} moiety as described here or elsewhere herein.

For compound groups where there is no double bond at the 2-position, exemplary substituents in the α-configuration or the β-configuration for R^{10A} are substituents described herein, e.g., —H, —²H, —³H, —OH, —OR^{PR}, —SH, —SR^{PR}, —NH₂, —NHR^{PR}, —NH—C1-6 alkyl, —NHCH₃, —N(CH₃)₂, —N₃, —NO₂, —CN, —SCN, —F, —Cl, —Br, —I, C1-6 optionally substituted alkyl, C1-6 optionally substituted alkylamine, C1-6 ether, C1-6 ester, C1-6 thioether, C1-6 thioester, optionally substituted monosaccharide, sulfate, sulfate ester, phosphate, phosphate ester, carbamate or carbonate such as —OC(O)—CH₃, —OC(O)—C₂H₅, —SC(O)—CH₃, —SC(O)—C₂H₅, —OCH₃, —OC₂H₅, —SCH₃, —SC₂H₅, —NHC(O)—O—CH₃, —NHC(O)—O—C₂H₅, —OC(O)—NH₂, —OC(O)—NHCH₃, —OC(O)—NHC₂H₅, —OC(O)—OCH₃, —OC(O)—OC₂H₅. R^{10A} can also be a double bonded moiety, e.g., ═O, ═S, ═NOH or ═CH₂, when there is no double bond at the 2-position.

For compound groups where there is no double bond at the 4-position, exemplary substituents in the α-configuration or the β-configuration for R^{10B} are substituents described herein, e.g., —H, —²H, —³H, —OH, —OR^{PR}, ═O, —SH, —SR^{PR}, ═S, —NH₂, —NHR^{PR}, —NH—C1-6 alkyl, —NHCH₃, —N(CH₃)₂, —F, —Cl, —Br, —I, C1-6 optionally substituted alkyl, C1-6 optionally substituted alkylamine, C1-6 ether, C1-6 ester, C1-6 thioether, C1-6 thioester, optionally substituted monosaccharide, sulfate, sulfate ester, phosphate, phosphate ester, carbamate or carbonate such as —OC(O)—CH₃, —OC(O)—C₂H₅, —SC(O)—CH₃, —SC(O)—C₂H₅, —OCH₃, —OC₂H₅, —SCH₃, —SC₂H₅, —CH₃, —C₂H₅, —NHC(O)—O—CH₃, —NHC(O)—O—C₂H₅, —OC(O)—NH₂, —OC(O)—NHCH₃, —OC(O)—NHC₂H₅, —OC(O)—OCH₃, —OC(O)—OC₂H₅. R^{10B} can also be a double bonded moiety, e.g., ═O, ═S, ═NOH or ═CH₂, when there is no double bond at the 4-position.

For compound groups where there is no double bond at the 6-position, exemplary substituents in the α-configuration or the β-configuration for R^{10C} are substituents described herein, e.g., —H, —²H, —³H, —OH, —OR^{PR}, ═O, —SH, —SR^{PR}, ═S, —NH₂, —NHR^{PR}, —NH—C1-6 alkyl, —NHCH₃, —N(CH₃)₂, —F, —Cl, —Br, —I, C1-6 optionally substituted alkyl, C1-6 optionally substituted alkylamine, C1-6 ether, C1-6 ester, C1-6 thioether, C1-6 thioester, optionally substituted monosaccharide, sulfate, sulfate ester, phosphate, phosphate ester, carbamate or carbonate such as -β-D-glucopyranoside, —OC(O)—CH₃, —OC(O)—C₂H₅, —SC(O)—CH₃, —SC(O)—C₂H₅, —OCH₃, —OC₂H₅, —SCH₃, —SC₂H₅, —CH₃, —C₂H₅, —NHC(O)—O—CH₃, —NHC(O)—O—C₂H₅, —OC(O)—NH₂, —OC(O)—NHCH₃, —OC(O)—NHC₂H₅, —OC(O)—OCH₃, —OC(O)—OC₂H₅. R^{10C} can also be a double bonded moiety, e.g., ═O, ═S, ═NOH or ═CH₂, when there is no double bond at the 6-position.

For any of the foregoing independently selected R^{10A}, R^{10B} and/or R^{10C} substituents, R^{10D} can be any one of these single bonded substituents in the α- or β-configuration or another single bonded R^{10D} substituent described elsewhere herein in the α- or β-configuration, e.g., α-OH, β-OH, α-F, β-F, α-C1-6 optionally substituted alkyl or β-Cl-6 optionally substituted alkyl. R^{10D} can also be a double bonded moiety, e.g., ═O, ═S, ═NOH, ═CH₂ or ═CHCH₂OH, as described herein.

Group 57. This group comprises compounds in the compound groups 1 through 56-55-54-53-52-51-50-47 described above, wherein 1, 2, 3 or 4 of R¹, R², R³ and R⁴ are a moiety defined herein other than one of the moieties listed in Table A, with exemplary moieties as described in the following paragraphs (1) through (15). Moieties or groups listed in paragraphs (1) through (15) such as optionally substituted alkyl, optionally substituted alkylamine, O-linked carbamate, N-linked carbamate and N-linked amino acid ester include the exemplary groups described (a) in the following paragraphs and (b) elsewhere herein. Optionally substituted alkyl groups for any of the moieties described in paragraphs (1) through (12) will typically be a C1-20, a C1-12 or a C1-6 optionally substituted alkyl group that is (i) optionally substituted with 1, 2, 3, 4, 5, 6 or more independently selected substitutions as described herein and (ii) saturated or unsaturated with 1, 2, 3 or more independently selected —CH₂═CH₂—, —CHR^{10A}═CHR^{10B}—, —CH₂≡CH₂—, —CHR^{10A}≡CHR^{10B} , where R^{10K} and R^{10L} independently are an R¹⁰ moiety as defined for F1Cs, e.g., they can be independently selected —H, C1-C6 optionally substituted alkyl, C1-6 ether, C1-6 thioether, —NH—C1-6 optionally substituted alkyl, halogen or another R¹⁰ moiety described elsewhere herein. Similarly, other organic moieties, e.g., carbamates, esters, thioesters or carbonates, will typically be a C1-20, a C1-12 or a C1-6 organic moiety that is optionally substituted with 1, 2, 3, 4, 5, 6 or more independently selected substitutions as described herein, e.g., for substituted alkyl groups.

(1) Compounds in any of the foregoing groups 1 through 56-55-54-53-52-51-50-47 where R¹⁰ moieties 1 through 10 in Table A are replaced with the following moieties: 1 is -Z-optionally substituted alkyl, 2 is an ester (e.g., —O—C(O)—(CH₂)ₙ—CH₃, —O—C(O)—(CH₂)ₙ—NH₂, —O—C(O)—(CH₂)ₙ—N(R^{PR})₂, —O—C(O)—(CH₂)ₙ—CH₂ZR^{PR}, —O—C(O)—CH(ZR^{PR})—(CH₂)ₙ—CH₃ or another ester described herein, where n is 0, 1, 2, 3, 4, 5, 6, 7 or 8, Z independently are —NH—, oxygen or sulfur and R^{PR} independently or together are —H, a protecting group or a counterion, e.g., methoxymethyl, —CH₃ or —C₂H₅), 3 is a thioester (e.g., —S—C(O)—(CH₂)ₙ—CH₃, —S—C(O)—(CH₂)ₙ—NH₂, —S—C(O)—(CH₂)ₙ—NHR^{PR}, —S—C(O)—(CH₂)ₙ—CH₂ZR^{PR}, —S—C(O)—CH(ZR^{PR})—(CH₂)ₙ—CH₃ or another thioester described herein, where n is 0,1, 2, 3, 4, 5, 6, 7 or 8, Z independently are —NH—, oxygen (—O—) or sulfur (—S—) and R^{PR} is —H or a protecting group, e.g., —CH₃ or —C₂H₅), 4 is a carbonate (e.g., —O—C(O)—O-optionally substituted alkyl), 5 is optionally substituted alkylamine (e.g., —NH-optionally substituted alkyl), 6 is optionally substituted dialkylamine (e.g., —N(optionally substituted alkyl)₂, where each optionally substituted alkyl is independently chosen), 7 is an N linked carbamate (e.g., —NH—C(O)—O-optionally substituted alkyl or —NH—C(O)—OH), 8 is an O linked carbamate (e.g., —O—C(O)—NH₂ or —O—C(O)—NH-optionally substituted alkyl), 9 is —O-optionally substituted monosaccharide and 10 is —H. Exemplary optionally substituted alkyl groups for any of these moieties include —CH₂CH₂—O—CH₃, —CH₂CH₂—S—CH₃, —CH(ZR^{PR})—(CH₂)ₙ—CH₂ZR^{PR}, —CH(ZR^{PR})—(CH₂)ₙ—CH₂NHR^{PR}, —CH₂—(CH₂)ₙ—C(O)OR^{PR}, —CH₂—(CH₂)ₙ—C(O)SR_{PR}, —CH₂—(CH₂)ₙ—C(O)NHR^{PR}, or any alkyl, alkenyl or alkynyl moiety described herein, e.g., any of which optionally having 1, 2, 3, 4, 5, 6, 7, 8, 9, 10, 11, 12 or more carbon atoms, with any of these being optionally substituted with 1, 2, 3, 4, 5, 6 or more independently selected substitutions, where n and Z are as described above.

(2) Compounds in any of the foregoing groups 1 through 56-55-54-53-52-51-50-47 where R¹ moieties 1 through 10 in Table A are replaced with the following moieties: 1 is —O-optionally substituted disaccharide, 2 is an N-linked amino acid, an N-linked amino acid ester or a salt (e.g., —NH—(CH₂)ₙ—C(O)OH, —NH—(CH₂)ₙ—C(O)OR^{PR}, —NH—(CH₂)ₙ—C(O)OCH₃, —NH—CH(CH₃)—C(O)OR^{PR}, —NH—CH(CH₂OH)—C(O)OR^{PR} or —NH—CH₂—CH₂—C(O)OR^{PR}, where R^{PR} is —H, a counter ion or a protecting group and chiral carbon atoms are in the D-, L- or DL-configuration), 3 is an O-linked amino acid, an O-linked amino acid ester, or a salt of any of these (e.g., —O—C(O)—(CH₂)ₙ—NHR^{PR}, —O—CH₂—NH₂, —O—CH₂—CH₂—NH₂, —O—C(O)—CH₂—CH₂—NHR^{PR}—, —O—C(O)—(CH₂)ₙ—NH—C1-C6 optionally substituted alkyl, —O—C(O)—O—(CH₂)ₙ—NH—C1-C6 optionally substituted alkyl, —O—C(O)—CH(CH₃)—(CH₂)ₙ—NH₂, —O—C(O)—CH(CH₃)—(CH₂)ₙ—NHR^{PR} or —O—C(O)—CH(CH₂OH)—(CH₂)ₙ—NH₂, where R^{PR} is —H, a counter ion or a protecting group and chiral carbon atoms are in the D-, -L or -DL configuration), 4 is an S-linked amino acid, an S-linked amino acid ester or a salt (e.g., —S—C(O)—CH₂—NHR^{PR}, —S—C(O)—CH₂—NH₂, —S—C(O)—CH₂—CH₂—NHR^{PR}, —S—C(O)—(CH₂)ₙ—NH₂, —S—C(O)—(CH₂)ₙ—NHR^{PR}, —S—C(O)—CH(CH₂OH)—(CH₂)ₙ—NH₂, —S—C(O)—(CH₂)ₙ—NH—C1-C6 optionally substituted alkyl, where R^{PR} is —H, a counter ion or a protecting group and chiral carbon atoms are in the D-, -L or -DL configuration), 5 is a sulfate ester (e.g., —O—S(O)(OR^{PR})—O-optionally substituted alkyl), 6 is —O—S(O)—O-optionally substituted alkyl, 7 is —F, —Cl —Br or —I, 8 is a polymer or polymer mixture such as one, two or more of PEG-100, PEG-200, PEG-300 or PEG-400, 9 is an N-linked heterocycle (e.g., N-morpholino, N-pyrrolidinyl or N-piperidinyl) and 10 is a C-linked heterocycle, e.g., 2-pyrimidinyl or 2-piperidinyl, where for any of these moieties, n is 1, 2, 3, 4, 5, 6, 7, 8, 9, 10, 11, 12 and R^{PR} is a protecting group or optionally substituted alkyl such as —CH₃, —CF₃ or —C₂H₅. When R¹ is a polymer, exemplary compounds have structures such as steroid 3-position-O—C(P)—(OCH₂—CH₂)ₘ—OH, steroid 3 steroid 3-position-O—C(O)—(OCH₂—CH₂)_{m—CH}₃, steroid 3-position-S—C(O)—(OCH₂—CH₂)ₘ—OH, steroid 3-position-S—C(O)—(OCH₂—CH₂)ₘ—OR^{PR}, steroid 3-position-S—C(O)—(OCH₂—CH₂)ₘ—CH₃, where the polymer is in the α- or β-configuration when no double bond is present at the 3-position and m is one, two or more of about 1, 2, 3, 4, 5, 6, 7, 8, 9, 10, 11, 12, 13, 14, 15, 16, 18, 20, 22, 25, 30, 35, 40, 45, 50, 55, 60 or more or where the average value of m is one of these integers.

(3) Compounds in any of the foregoing groups 1 through 56-55-54-53-52-51-50-47 where there is no double bond at the 2-3 or 3-4 position and R¹ moieties 1 through 10 in Table A are replaced with the following moieties: 1 is ═O, 2 is =S, 3 is ═NOH, 4 is ═NOCH₃, 5 is ═NOC₂H₅, 6 is ═N-optionally substituted alkyl, 7 is ═NO-optionally substituted alkyl, 8 is ═NH, 9 is ═CH₂ and 10 is ═C-optionally substituted alkyl. Exemplary group 57(3)-6 (i.e., group 57 paragraph 3 compounds from group 6, which described 1,5-dienes) compounds include compound 1.2.4.1, which is 3-oxo-7β-hydroxy-16α-fluoro-17β-aminoandrost-1,5-diene, 1.1.4.1, which is 3-oxo-16α-fluoro-17β-aminoandrost-1,5-diene, 1.1.5.9, which is 3-oxo-17β-hydroxyandrost-1,5-diene, 1.1.7.1, which is 3-oxo-16α-acetoxy-17β-aminoandrost-1,5-diene and compound 1.1.1.10, which is 3β-hydroxy-16α-bromo-17β-acetoxyandrost-1,5-diene and 16α-hydroxy, 16α-methyl, 16α-amino, 16α-aminomethyl, 16α-acetate and 16α-halo analogs of any of these compounds. Exemplary group 57-7 compounds include compound 1.2.4.1, which is 3-oxo-7-hydroxy-16α-fluoro-17β-aminoandrost-1,6-diene, 1.1.4.1, which is 3-oxo-16α-fluoro-17β-aminoandrost-1,6-diene, 1.1.5.9, which is 3-oxo-17β-dihydroxyandrost-1,6-diene, 1.1.7.1, which is 3-oxo-16α-acetoxy-17β-aminoandrost-1,6-diene and compound 1.1.1.10, which is 3β-hydroxy-16α-bromo-17β-acetoxyandrost-1,6-diene and 16α-hydroxy, 16α-methyl, 16α-amino, 16α-aminomethyl, 16α-acetate and 16α-halo analogs of any of these compounds. Exemplary group 57-8 compounds include compound 1.2.4.1, which is 3-oxo-7-hydroxy-16α-fluoro-17β-amino-5β-androst-1,6-diene, 1.1.4.1, which is 3-oxo-16α-fluoro-17β-amino-5β-androst-1,6-diene, 1.1.5.9, which is 3-oxo-17β-dihydroxy-5β-androst-1,6-diene, 1.1.7.1, which is 3-oxo-16α-acetoxy-17β-amino-5β-androst-1,6-diene and compound 1.1.1.10, which is 3β-hydroxy-16α-bromo-17β-acetoxy-5β-androst-1,6-diene and 16α-hydroxy, 16α-methyl, 16α-amino, 16α-aminomethyl, 16α-acetate and 16α-halo analogs of any of these compounds. Other group 57 compounds include of any of these compounds where R¹ is in the α-configuration, and/or R³ is in the β-configuration and/or R⁴ is in the α-configuration and/or R⁵ is a moiety other than methyl, e.g., —CH₂OH, —CHO, —C₂H₅, —C₃H₇ or another R⁵ described herein and/or R⁶ is a moiety other than methyl, e.g., —H, —F, —Cl, —OH, —SH, —NH₂, —NH^{PR}, an ester or ether, —CH₂OH, —C—C≡CH, —C₂H₅, —C₃H₇ or another R⁶ described herein and/or R^{10G} is a moiety other than —H, e.g., —F, —Cl, —Br, —CH₃, —OH, —SH, —NHR^{PR} or another R^{10G} moiety described herein and/or R⁸ is a moiety other than methylene, e.g., —O—, —S—, —NH—, ═N—, —N(CH₃)—, —N(C₂H₅)—, —CH(α-optionally substituted C1-C6 alkyl)-, —CH(β-optionally substituted C1-C6 alkyl)-, —CH(α—OH)—, —CH(β-OH)—, —C(O)—, —CH(α-SH)—, —CH(β-SH)—, —CH(α-F)—, —CH(β-F)—, —CH(α-I)—, —CH(β-I)— or another R⁸ moiety described herein or R⁸ is absent, leaving a 5-membered ring and/or R⁹ is a moiety other than methylene, e.g., —O—, —S—, —NH—, —N(CH₃)—, —N(C₂H₅)—, —CH(α-optionally substituted C1-C6 alkyl)-, —CH(β-optionally substituted C1-C6 alkyl)-, —CH(α-OH)—, —CH(β-OH)—, —C(O)—, —CH(α-SH)—, —CH(β-SH)—, —CH(α-F)—, —CH(β-F)—, —CH(α-I)—, —CH(PβI)— or another R⁹ moiety described herein or R⁹ is absent, leaving a 5-membered ring. Other exemplary compounds include analogs of any of these compounds where (i) R⁷ is another R⁷ moiety described herein such as —O—, —NH—, ═N—, —NCH₃—, —NC₂H₅—, —CH═CH—, —CR¹⁰═CR¹⁰—, —CH₂—CH(α-R¹⁰)—, —CH₂—CH(β-R¹⁰)—, —O—, —CH₂—C(β-R¹⁰)(α-R¹⁰)—, —C(β-R¹⁰)(α-R¹⁰)—, where R¹⁰ independently or together are —OH, ═O, —NH₂, —NHR^{PR}, —SH, halogen, —C(O)—OR^{PR}, an ester, an ether, C1-C8 optionally substituted alkyl, a heterocycle, a monosaccharide, a polymer or another R¹⁰ moiety described herein or (ii) R^{10H} is a moiety other than —H such as —OH, —OR^{PR}, —SH, —SR^{PR}, —NH₂, —NHR^{PR}, —NHCH₃, —N(CH₃)₂, —CH₃ or C1-C6 optionally substituted alkyl. Other groups and analogous compounds include those in group 57-9, 57-10, 57-11, 57-12, 57-13, 57-14, 57-15, 57-16, 57-17, 57-18, 57-19, 57-20, 57-21, 57-22, 57-23, 57-24, 57-30, 57-31, 57-32, 57-33, 57-40, 57-41, 57-42, 57-43, 57-44, 57-45, 57-46, 57-47, 57-48, 57-49 and analogs or epimers where R¹ is ═O, ═S or ═NOH, and/or R² is in the α-configuration, and/or R³ is in the β-configuration and/or R⁴ is in the α-configuration and/or R⁵ is a moiety other than methyl such as —H, ethyl, ethynyl, 1-propynyl or C2-C6 optionally substituted alkyl and/or R⁶ is a moiety other than methyl such as —H, —F, —Cl, —Br, —OH, —SH, —NH₂, —NHR^{PR}, ethyl, ethynyl, 1-propynyl or C2-C6 optionally substituted alkyl and/or R^{10G} is a moiety other than —H such as —F or —Cl, and/or R⁸ is a moiety other than methylene or R⁸ is absent, leaving a 5-membered ring and/or R⁹ is a moiety other than methylene or R⁹ is absent, leaving a 5-membered ring. In any of these compounds, R^{PR} in dependently or together are —H or a protecting group.

(4) Compounds in any of the foregoing groups 1 through 56-55-54-53-52-51-50-47 where R¹ moieties 1 through 10 in Table A are replaced with the following moieties: 1 is a phosphate, phosphate ester or a salt, e.g., —O—P(O)(OH)—OH, —O—P(O)(OH)—O⁻Na⁺, —O—P(O)(OH)—O-optionally substituted alkyl, —O—P(O)(OR^{PR})—O-optionally substituted alkyl, 2 is a thiophosphate or thiophosphate ester, 3 is a sulfamate, 4 is a phosphonate, 5 is a thiophosphonate, 6 is a sulfonate, 7 is a polymer, 8 is an optionally substituted oligosaccharide, 9 is a thionoester and 10 is an amide. Exemplary R¹ moieties include (i) —O—P(O)(O—(C(O))ₘ—(CH₂)ₙ—CH₃)—OH, —O—P(O)(O—(C(O))ₘ—(CH₂)ₙ—CH₃)—O—(CH₂)ₙ—CH₃ where m independently are 0 or 1 and n independently are 1, 2, 3, 4, 5, 6, 7, 8, 9, 10 or 11, (ii) —O—P(O)(SH)—OH, —O—P(O)(SH)—O⁻Na⁺, —O—P(O)(OH)—S-optionally substituted alkyl, —O—P(O)(S—(C(O))ₘ—(CH₂)ₙ—CH₃)—OH, —O—P(O)(S—(C(O))ₘ—(CH₂)ₙ—CH₃)—O—(CH₂)ₙ—CH₃ where m independently are 0 or 1 and n independently are 1, 2, 3, 4, 5, 6, 7, 8, 9, 10 or 11, (iii) —(OCH₂HC₂)ₙ—OH, —(OCH₂HC₂)ₙ—CH₃, —(OCH₂HC₂)ₙ—OR^{PR}, —(OCH₂HC₂)ₙ—SH, —(OCH₂HC₂)ₙ—SR^{PR}, —(OCH₂HC₂)ₙ—NH₂ or —(OCH₂HC₂)ₙ—NHR^{PR} where n is an integer such as an integer from about 4, 8, 12 or 20 to about 30, 40, 50 or 100, (vi) —O—S(O)(O)—NH—(C(O))ₘ—(CH₂)ₙ—X—(CH₂)ₙ—CH₃, —O—S(O)(O)—NH—(C(O))ₘ—(CH₂)ₙ—CH₃, —O—S(O)(O)—NH—(CH₂)ₙ—X—CH₃, —O—S(O)(O)—NH—(CH₂)ₙ—(C(O))ₘ—X—(C(O))ₘ—(CH₂)ₘ—CH₃, —O—S(O)(O)—NH₂, —O—S(O)(O)—NH—C1-C8 optionally substituted alkyl, —O—S(O)(O)—N—(C1-C8 optionally substituted alkyl)₂, —NH—S(O)(O)—O—(CH₂)ₙ—X—CH₃, —NH—S(O)(O)—O—(C(O))ₘ—(CH₂)ₙ—X—CH₃, —NH—S(O)(O)—O—(CH₂)ₙ—(C(O))ₘ—X—CH₃, —NH—S(O)(O)—O—(CH₂)ₙ—X—(C(O))ₘ—CH₃ or —NH—S(O)(O)—O—(CH₂)ₘ-optionally substituted heterocycle, where X is —O—, —S—, —NH—, —N(C1-C8 optionally substituted alkyl)-, m independently are 0 or 1, n independently are 1, 2, 3, 4, 5, 6, 7, 8, 9, 10 or 11 and optionally substituted alkyl are each independently selected, (vii) —O—S(O)(O)—(C(O))ₘ—(CH₂)ₙ—X—(CH₂)ₙ—CH₃, —O—S(O)(O)—(C(O))ₘ—(CH₂)ₙ—CH₃, —O—S(O)(O)—(CH₂)ₙ—X—CH₃, —O—S(O)(O)(CH₂)ₙ—(C(O))ₘ—X—(C(O))ₘ—(CH₂)ₘ—CH₃, —O—S(O)(O)—CH₃, —O—S(O)(O)—C1-C8 optionally substituted alkyl, —S(O)(O)—O—(CH₂)ₙ—X—CH₃, —S(O)(O)—O—(C(O))ₘ—(CH₂)ₙ—X—CH₃, S(O)(O)—O—(CH₂)ₙ—(C(O))ₘ—X—CH₃, —S(O)(O)—O—(CH₂)ₙ—X—(C(O))ₘ—CH₃ or —S(O)(O)—O—(CH₂)ₘ-optionally substituted heterocycle, where X is —O—, —S—, —NH—, —N(C1-C8 optionally substituted alkyl)-, m independently are 0 or 1, n independently are 1, 2, 3, 4, 5, 6, 7, 8, 9, 10 or 11 and optionally substituted alkyl are each independently selected, (viii) —O—P(O)(OR^{PR})—(C(O))ₘ—(CH₂)ₙ—X—(CH₂)ₙ—CH₃, —O—P(O)(OR^{PR})—(C(O))ₘ—(CH₂)ₙ—CH₃, —O—P(O)(OR^{PR})—(CH₂)ₙ—X—CH₃, —O—P(O)(OR^{PR})—(CH₂)ₙ—(C(O))ₘ—X—(C(O))ₘ—(CH₂)ₘ—CH₃, —O—P(O)(OR^{PR})—CH₃, —O—P(O)(OR^{PR})C1-C8 optionally substituted alkyl, —P(O)(OR^{PR})—O—(CH₂)ₙ—X—CH₃, —P(O)(OR^{PR})—O—(C(O))ₘ—(CH₂)ₙ—X—CH₃, —P(O)(OR^{PR})—O—(CH₂)ₙ—(C(O))ₘ—X—CH₃, —P(O)(OR^{PR})—O—C1-C8 optionally substituted alkyl or —P(O)(OR^{PR})—O—(CH₂)ₘ-optionally substituted heterocycle, where X is —O—, —S—, —NH—, —N(C1-C8 optionally substituted alkyl)-, m independently are 0 or 1, n independently are 1, 2, 3, 4, 5, 6, 7, 8, 9, 10 or 11, R^{PR} independently are —H or a protecting group and optionally substituted alkyl are each independently selected, (ix) —O—P(S)(OR^{PR})—(C(O))ₘ—(CH₂)ₙ—X—(CH₂)ₙ—CH₃, —O—P(S)(OR^{PR})—(C(O))ₘ—(CH₂)ₙ—CH₃, —O—P(S)(OR^{PR})—(CH₂)ₙ—X—CH₃, —O—P(S)(OR^{PR})(CH₂)ₙ—(C(O))ₘ—X—(C(O))ₘ—(CH₂)ₘ—CH₃, —O—P(S)(OR^{PR})—CH₃, —O—P(S)(OR^{PR})—C1-C8 optionally substituted alkyl, —P(S)(OR^{PR})—O—(CH₂)ₙ—X—CH₃, —P(S)(OR^{PR})—O—(C(O))ₘ—(CH₂)ₙ—X—CH₃, —P(S)(OR^{PR})—O—(CH₂)ₙ—(C(O))ₘ—X—CH₃, —P(S)(OR^{PR})—O—C1-C8 optionally substituted alkyl or —P(S)(OR^{PR})—O—(CH₂)ₘ-optionally substituted heterocycle, where X is —O—, —S—, —NH—, —N(C1-C8 optionally substituted alkyl)-, m independently are 0 or 1, n independently are 1, 2, 3, 4, 5, 6, 7, 8, 9, 10 or 11, R^{PR} independently are —H or a protecting group and optionally substituted alkyl are each independently selected and (x) —C(O)—NH—(C(O))ₘ—(CH₂)ₙ—X—(CH₂)ₙ—CH₃, —C(O)—NH—(C(O))ₘ—(CH₂)ₙ—CH₃, —C(O)—NH—(CH₂)ₙ—X—CH₃, —C(O)—NH—(CH₂)ₙ—(C(O))ₘ—X—(C(O))ₘ—(CH₂)ₘ—CH₃, —C(O)—NH—CH₃, —C(O)—NH—C1-C8 optionally substituted alkyl, —C(O)—NH—CH₂—CH₂—CH₃, —C(O)—NH—CH₂OR^{PR}, —C(O)—NH—CH₂—CH₂—CH₂OR^{PR}, —C(O)—NH—CH₂—CH₂—CH₂—CH₃, —C(O)—NH—CH₂—CH₂—CH₂—CH₂C(O)OR^{PR}, —C(O)—NH—CH₂—CH₂C(O)OR^{PR}, —NH—C(O)—(CH₂)ₙ—X—CH₃, —NH—C(O)—(C(O))ₘ—(CH₂)ₙ—X—CH₃, —NH—C(O)—(CH₂)ₙ—(C(O))ₘ—X—CH₃, or —NH—C(O)—(CH₂)ₘ-optionally substituted heterocycle, where X is —O—, —S—, —NH—, —N(C1-C8 optionally substituted alkyl)-, m independently are 0 or 1, n independently are 1, 2, 3, 4, 5, 6, 7, 8, 9, 10 or 11, R^{PR} independently are —H or a protecting group and optionally substituted alkyl are each independently selected.

(5) Compounds in any of the foregoing groups 1 through 56-55-54-53-52-51-50-47 and in paragraphs (1), (2), (3) and (4) in this group 57 where R⁴ moieties 1 through 10 in Table A are replaced with the following moieties: 1 is —O-optionally substituted alkyl, 2 is an ester (e.g., —O—C(O)—CH₃, —O—C(O)—CH₂CH₃, —O—C(O)—(CH₂)ₙ—CH₃, —O —C(O)—CF₃, —O—C(O)—(CH₂)ₙ—CF₃, —C(O)—(CH₂)ₙ—C(O)OR^{PR}, —O—C(O)CH₂—C(O)OR^{PR}, —O—C—(O)—(CH₂)₂—C(O)OR^{PR}, —O—C(O)—(CH₂)₃—C(O)OR^{PR}, —O—C(O)—(CH₂)₄—C(O)OR^{PR}, —O—C(O)—(CH₂)ₙ—NH₂, —O—C(O)—(CH₂)ₙ—N(R^{PR})₂, —O—C(O)—(CH₂)ₙ—CH₂ZR^{PR}, —O—C(O)—CH(ZR^{PR})—(CH₂)ₙ—CH₃ or another ester described herein, where n is 0, 1, 2, 3, 4, 5, 6, 7 or 8, Z is —O—, —NH— or —S— and R^{PR} independently or together are —H, a protecting group or a counter ion, e.g., methoxymethyl, Na⁺, K⁺, —CH₃ or —C₂H₅), 3 is a thioester (e.g., —S—C(O)—(CH₂)ₙ—CH₃, —S—C(O)—(CH₂)ₙ—NH₂, —S—C(O)—(CH₂)ₙ—NHR^{PR}, —S—C(O)—(CH₂)ₙ—CH₂ZR^{PR}, —S—C(O)—CH(ZR^{PR})—(CH₂)ₙ—CH₃ or another thioester described herein, where n is 0, 1, 2, 3, 4, 5, 6, 7 or 8, Z is oxygen or sulfur and R^{PR} is —H or a protecting group, e.g., —CH₃ or —C₂H₅), 4 is a carbonate (e.g., —O—C(O)—O-Optionally substituted alkyl), 5 is optionally substituted alkylamine (e.g., —NH-Optionally substituted alkyl), 6 is optionally substituted dialkylamine (e.g., —N(Optionally substituted alkyl)₂, where each optionally substituted alkyl is independently chosen), 7 is an N linked carbamate (e.g., —NH—C(O)—O-Optionally substituted alkyl or —NH—C(O)—OH), 8 is an O linked carbamate (e.g., —O—C(O)—NH₂ or —O—C(O)—NH-Optionally substituted alkyl), 9 is —O-optionally substituted monosaccharide and 10 is —H. Exemplary optionally substituted alkyl moieties include any such moiety described herein for any variable group and moieties such as —CF₃, —CF₂CF₃, —CH₂CF₃, —CF₂CF₂CF₃, —CH₂CH₂CF₃, —CF₂CF₂CF₂CF₃, —C(O)—CH₃, —C(O)—C₂H₇, —C(O)—C₄H₉, —C(O)—C₆H₁₃, —CH(OH)—CH₃, —CH(OH)—C₂H₅, —CH(OH)—C₃H₇, —CH(OH)—C₄H₉, —CH(OH)—C₆H₁₃, —C(O)—C₂H₄OR^{PR}, —C(O)—C₃H₆OR^{PR}, —C(O)—C₄H₈OR^{PR}, —C(O)—C₆H₁₃OR^{PR}, —C(O)—C₂H₄SR^{PR}, —C(O)—C₃H₆SR^{PR}, —C(O)—C₄H₈SR^{PR}, —C(O)—C₆H₁₃SR^{PR}, —C(O)—C₂H₄NHR^{PR}, —C(O)—C₃H₆NHR^{PR}, —C(O)—C₄H₈NHR^{PR}, —C(O)—C₆H₁₃NHR^{PR}, —C(O)OR^{PR}, —CH₂C(O)OR^{PR}, —CH₂CH₂C(O)OR^{PR}, —C(O)—O—CH₂C(O)OR^{PR}, —C(O)—O—CH₂CH₂C(O)OR^{PR}, —C(O)—O—CH₂CH₂CH₂C(O)OR^{PR}, where R^{PR} is —H, a protecting group or a counter ion such as Cl⁻, Na⁺ or K⁺.

(6) Compounds in any of the foregoing groups 1 through 56-55-54-53-52-51-50-47 and in paragraphs (1), (2), (3) and (4) in this group 57 where R⁴ moieties 1 through 10 in Table A are replaced with the following moieties: 1 is —O-optionally-substituted disaccharide, 2 is an N-linked amino acid, an N-linked amino acid ester or a salt (e.g., —NH—CH₂—C(O)OH, —NH—CH₂—C(O)OR^{PR}, —NH—CH₂—C(O)OCH₃, —NH—CHCH₃—C(O)OR^{PR} or —NH—CH₂—CH₂—C(O)OR^{PR}, where R^{PR} is —H, a counter ion or a protecting group and chiral carbon atoms are in the D-, -L or -DL configuration), 3 is an O-linked amino acid, an O-linked amino acid ester or a salt (e.g., —O—C(O)—CH₂—NHR^{PR}, —O—CH₂—NH₂, or —O—C(O)—CH₂—CH₂—NHR^{PR}, where R^{PR} is —H, a counter ion or a protecting group and chiral carbon atoms are in the D-, -L or -DL configuration), 4 is an S-linked amino acid, an S-linked amino acid ester or a salt (e.g., —S—C(O)—CH₂—NHR^{PR}, —S—CH₂—NH₂, or —S—C(O)—CH₂—CH₂—NHR^{PR}, where R^{PR} is —H, a counter ion or a protecting group and chiral carbon atoms are in the D-, -L or -DL configuration), 5 is a sulfate ester (e.g., —O—S(O)(OR^{PR})—O-Optionally substituted alkyl), 6 is —O—S(O)—O-Optionally substituted alkyl, 7 is a halogen such as —Br or —I, 8 is a halogen such as —F or —Cl, 9 is an N-linked heterocycle (e.g., N-morpholino) and 10 is a C-linked heterocycle (e.g., 2-pyrimidinyl).

(7) Compounds in any of the foregoing groups 1 through 56-55-54-53-52-50-47 and in paragraphs (1), (2), (3) and (4) in this group where there is no double bond at the 16-17 position and R⁴ moieties 1 through 10 in Table A are replaced with the following moieties: 1 is ═O, 2 is ═S, 3 is ═NOH, 4 is ═NOCH₃, 5 is ═NOC₂H₅, 6 is ═N-optionally substituted alkyl, 7 is ═NO-optionally substituted alkyl, 8 is ═NH, 9 is ═CH₂ and 10 is ═C-optionally substituted alkyl. Exemplary compounds and compound genera include 3β-amino-17-oxoandrost-5(10)-ene, 3α-amino-17-oxoandrost-5(10)-ene, 3,17-dioxoandrost-5(10)-ene, 3β-hydroxy-3α-methyl-17-oxoandrost-5(10)-ene, 3α-hydroxy-3β-ethynyl-17-oxoandrost-5(10)-ene, 3β-mercapto-17-oxoandrost-5(10)-ene, 3α-mercapto-17-oxoandrost-5(10)-ene, 3β-amino-17-oxoandrost-5,7-diene, 3α-amino-17-oxoandrost-5,7-diene, 3β-hydroxy-3α-methyl-17-oxoandrost-5,7-diene, 3α-hydroxy-3β-ethynyl-17-oxoandrost-5,7-diene, 3-amino-17-oxoandrost-1,3-diene, 3-hydroxy-17-oxoandrost-1,3-diene, 3-hydroxy-17-oxoandrost-1,3-diene, 3-amino-17-oxo-5β-androst-1,3-diene, 3-amino-17-oxo-5β-androst-1,3-diene, 3-hydroxy-17-oxo-5β-androst-1,3-diene, 3-hydroxy-17-oxo-5β-androst-1,3-diene, 3-amino-17-oxoandrost-2,5(10)-diene, 3-amino-17-oxoandrost-2,5(10)-diene, 3-hydroxy-17-oxoandrost-2,5(10)-diene, 3-hydroxy-17-oxoandrost-2,5(10)-diene, 3-amino-17-oxo-5β-androst-2,5(10)-diene, 3-amino-17-oxo-5β-androst-2,5(10)-diene, 3-hydroxy-17-oxo-5β-androst-2,5(10)-diene, 3-hydroxy-17-oxo-5β-androst-2,5(10)-diene, 3-amino-17-oxoandrost-2,5-diene, 3-amino-17-oxoandrost-2,5-diene, 3-hydroxy-17-oxoandrost-2,5-diene, 3-hydroxy-17-oxoandrost-2,5-diene, 3-amino-17-oxo-5β-androst-2,5-diene, 3-amino-17-oxo-5β-androst-2,5-diene, 3-hydroxy-17-oxo-5β-androst-2,5-diene, 3-hydroxy-17-oxo-5β-androst-2,5-diene, 3-amino-17-oxoandrost-1,3,5-triene, 3-hydroxy-17-oxoandrost-1,3,5-triene, 3-amino-17-oxoandrost-1,3,6-triene, 3-hydroxy-17-oxoandrost-1,3,6-triene, 3-amino-17-oxo-5β-androst-1,3,6-triene, 3-hydroxy-17-oxo-5β-androst-1,3,6-triene, 3-amino-17-oxoandrost-1,3,5(10)-triene, 3-hydroxy-17-oxoandrost-1,3,5(10)-triene, 3-amino-17-oxoandrost-1,3,5(10),8(14)-tetraene, 3-hydroxy-17-oxoandrost-1,3,5(10),8(14)-tetraene, 3-amino-17-oxoandrost-1,3,5(10),8(9)-tetraene, 3-hydroxy-17-oxoandrost-1,3,5(10),8(9)-tetraene, 3-amino-17-oxoandrost-1,3,5(10),6-tetraene, 3-hydroxy-17-oxoandrost-1,3,5(10),6-tetraene, 3-amino-17-oxoandrost-1,3,5(10),7-tetraene, 3-hydroxy-17-oxoandrost-1,3,5(10),7-tetraene, 3-amino-17-oxoandrost-1,3,5(10),15-tetraene, 3-hydroxy-17-oxoandrost-1,3,5(10),15-tetraene and an analog of any of these compounds wherein (i) the 3-position (R¹) is substituted with one or two independently selected R¹ moieties as described herein such as —SH, ═O, ═S, ester, ether, carbonate, thioester, thioether, polymer, O-linked carbamate, N-linked amide, N-linked carbamate, —NH—C1-C10 optionally substituted alkyl or —N(C1-C10 optionally substituted alkyl)₂ such as methyl, ethyl, propyl or butyl, or one or two other independently selected R¹ moieties described herein, instead of —OH, —SH or —NH₂, where each optionally substituted alkyl group is the same or different, and/or (ii) the 17-position (R⁴) is a double bonded moiety as described herein such as ═S, ═CH₂, ═CHCH₃, ═CHC₂H₅, ═C(OH)—C₂H₅, ═C(SH)—C₂H₅, ═C(OH)—CH₃, ═C(SH)—CH₃, ═CHCH₂OH, ═CHC₂H₄OH, ═CH—C1-C10 optionally substituted alkyl, ═NOH, ═NO—CH₃, ═NO—C1-C10 optionally substituted alkyl, ═N—CH₃, ═N—C1-C10 optionally substituted alkyl, ethylene ketal (—O—CH₂—CH₂—O—) or another double bonded moiety or group described herein, is present at the 17-position instead of ═O, and/or (iii) the 16-position (R³) is substituted with one or two independently selected moieties described herein such as —F, —Cl, —Br, —I, —OH, —NHCH₃, —N(CH₃)₂, —NHC₂H₅, —N(C₂H₅)₂, —NHC₃H₇, —N(C₃H₇)₂, —NHC₃H₅, —N(C₃H₅)₂, —NHC₄H₉, —N(C₄H₉)₂, ═O, ═S, ═CH₂, C1-C10 optionally substituted alkyl such as methyl, ethynyl or 1-propynyl, -heterocycle, —(CH₂)-heterocycle, a polymer, ═CHCH₃, ═CHC₂H₅, ═C(OH)—C₂H₅, ═C(SH)—C₂H₅, ═C(OH)—CH₃, ═C(SH)—CH₃, ═CHCH₂OH, ═CHC₂H₄OH, ═CH—C1-C10 optionally substituted alkyl, ═NOH, ═NO—CH₃, ═NO—C1-C10 optionally substituted alkyl, ═N—CH₃, ═N—C1-C10 optionally substituted alkyl, ═N—CH₂CH₃, ═N—CH₂CH₂OR^{PR}, ═N—CH₂CH₂SR^{PR}, ═N—CH₂CH₂NHR^{PR}, ethylene ketal and/or one or two other independently selected R³ moieties described herein, where the substituent(s) is in the α-configuration or the β-configuration when no double bond is present at the 16-position and R^{PR} is —H or a protecting group, and/or (iv) the 2-position (R⁹) is substituted with one or two independently selected substituents described herein such as —F, —Cl, —Br, —I, —OH, —OR^{PR}, —SH, —SR^{PR}, —NH₂, —NHR^{PR}, —NHCH₃, —N(CH₃)₂, —NHC₂H₅, —N(C₂H₅)₂, —NHC₃H₇, —N(C₃H₇)₂, —NHC₃H₅, —N(C₃H₅)₂, —NHC₄H₉, —N(C₄H₉)₂, ═O, ═S, ═CH₂, ═CHCH₃, ═CHC₂H₅, ═C(OH)—C₂H₅, ═C(SH)—C₂H₅, ═C(OH)—CH₃, ═C(SH)—CH₃, ═CHCH₂OH, ═CHC₂H₄OH, ═CH—C1-C10 optionally substituted alkyl, ═NOH, ═NO—CH₃, ═NO—C1-C10 optionally substituted alkyl, ═N—CH₃, ═N—C1-C10 optionally substituted alkyl, ═N—CH₂CH₃, ═N—CH₂CH₂OR^{PR}, ═N—CH₂CH₂SR^{PR}, ═N—CH₂CH₂NHR^{PR}, ═N—C1-C10 optionally substituted alkyl, ethylene ketal, C1-C10 optionally substituted alkyl such as methyl, ethynyl or 1-propynyl, C1-C10 alkoxy such as methoxy or ethoxy, -heterocycle, —(CH₂)-heterocycle, or a polymer where, when no double bond is present at the 2-poistion, the substituent(s) is in the α-configuration or the β-configuration, and/or (v) R^{10G} at the 9-position, when present, is —F, —Cl, —Br, —I, —OH, C1-C10 optionally substituted alkyl such as methyl, ethyl, ethynyl or 1-propynyl or cyclopropyl with the 11-position or another moiety described herein, and/or (vi) the 7-position (R²) is substituted with one or two independently selected substituents described herein such as —OH, ═O, ═S, ═CH₂, —NH₂, —NHCH₃, —N(CH₃)₂, —NHC₂H₅, —N(C₂H₅)₂, —NHC₃H₇, —N(C₃H₇)₂, —NHC₃H₉, —N(C₄H₉)₂, ═NOH, ═NO—CH₃, ═NO—C1-C10 optionally substituted alkyl, ═N—CH₃, ═N—C1-C10 optionally substituted alkyl, ═N—CH₂CH₃, ═N—CH₂CH₂OR^{PR}, ═N—CH₂CH₂SR^{PR}, ═N—CH₂CH₂NHR^{PR}, ═N—C1-C10 optionally substituted alkyl, ethylene ketal, —NH—C1-C10 optionally substituted alkyl such as hydroxymethyl, hydroxyethyl, hydroxypropyl or another optionally substituted alkyl described herein, —N(C1-C10 optionally substituted alkyl)₂, C1-C10 optionally substituted alkyl such as methyl, ethynyl, 1-propynyl or another optionally substituted alkyl described herein, -heterocycle, —(CH₂)-heterocycle, a polymer or one or two other substituents described elsewhere herein, where, when no double bond is present at the 7-poistion, the substituent(s) is in the α-configuration or the β-configuration, and/or (vii) the 6-position (R^{10C}) is substituted with a substituent such as —F, —Cl, —Br, —I, —OH, —NH₂, —NH—C1-C10 optionally substituted alkyl, —N(C1-C10 optionally substituted alkyl)₂ where each optionally substituted alkyl is one or two independently selected R¹, R⁴ or R^{10C} C1-C10 optionally substituted alkyl moieties described herein, ═O, ═S, ═CH₂, C1-C10 optionally substituted alkyl such as methyl, ethynyl, 1-propynyl or another optionally substituted alkyl described herein, -heterocycle, —(CH₂)-heterocycle, or a polymer where, when no double bond is present at the 6-poistion, the substituent is in the α-configuration or the β-configuration, and/or (viii) the 11-position (R⁸) is —O—, —S—, —NH—, —N(CH₃)—, —N(C₂H₅)—, —N(C₃H₇)—, ═N— or is substituted with one or two independently selected substituents described herein such as —F, —Cl, —Br, —I, —OH, ═O, —SH, ═S, ═CH₂, C1-C10 optionally substituted alkyl such as methyl, ethynyl or 1-propynyl, -heterocycle, —(CH₂)-heterocycle, a polymer or another R⁸ moiety described herein, where, when no double bond is present at the 11-poistion, the substituents are in the α-configuration or the β-configuration, e.g., R⁸ is —CH(α-C1-C10 optionally substituted alkyl)-, —CH(β-C1-C10 optionally substituted alkyl)-, —CH(β-F)—, —CH(α-F)—, —CF₂——CH(β-OH)—, —CH(α-OH)—, —C(O)—, —CH(β-SH)—, —CH(α-SH)—, —CH(β-NH₂)—, —CH(α-NH₂)—, —CH(β-NHCH₃)—, —CH(α-NHCH₃)—, —CH(β-N(CH₃)₂)—, —CH(α-N(CH₃)₂)—, —CH(β-NHC₂H₅)—, —CH(α-NHC₂H₅)—, —CH(α-heterocycle)-, —CH(β-heterocycle)-, —CH(α-polymer)-, —CH(β-polymer)-, —CH(α-ether)-, —CH(β-ether)-, —CH(α-thioether)-, —CH(β-thioether)-. Analogs of any of these compounds include compounds where substitutions described at two or three of (i), (ii), (iii), (iv), (v), (vi), (vii) and (viii) are present, e.g., substitutions as described at (i) and (ii), (i) and (iii), (i) and (iv), (i) and (vi), (i) and (vii), (i) and (viii), (i), (ii) and (iii), (i), (ii) and (vi), (i), (ii) and (v), (i), (ii) and (vi), (i), (ii) and (vii), (i), (ii) and (viii), (ii) and (iii), (ii) and (iv), (ii) and (v), (ii) and (vi), (ii) and (vii), (ii) and (viii), (i), (ii) and (iii), (i), (ii) and (iv), (i), (ii) and (v), (i), (ii) and (vi), (i), (ii) and (vii), (i), (ii) and (viii), (iii) and (iv), (iii) and (v), (iii) and (vi), (iii) and (vii), (iii) and (viii), (i), (iii) and (iv), (i), (iii) and (v), (i), (iii) and (vi), (i), (iii) and (vii), (i), (iii) and (viii), (iv) and (v), (iv) and (vi), (iv) and (vii), (iv) and (viii), (i), (iv) and (v), (i), (iv) and (vi), (i), (iv) and (vii), (i), (iv) and (viii), (v) and (vi), (v) and (vii), (v) and (viii), (i), (v) and (vi), (i), (v) and (vii), (i), (v) and (viii), (vi) and (vii), (vi) and (viii), (i), (vi) and (vii), (i), (vi) and (viii), (ii), (iii) and (iv), (ii), (iii) and (v), (ii), (iii) and (vi), (ii), (iii) and (vii) or at (ii), (iii) and (viii).

(8) Compounds in any of the foregoing groups 1 through 56-55-54-53-52-51-50-47 and in paragraphs (1), (2), (3) and (4) in this group 57 where R⁴ moieties 1 through 10 in Table A are replaced with the following moieties: 1 is a phosphate, phosphate ester or a salt, e.g., —O—P(O)(OH)—OH, —O—P(O)(OH)—O⁻Na⁺, —O—P(O)(OH)—O-optionally substituted alkyl —O—P(O)(OR^{PR})—O-optionally substituted alkyl, 2 is a thiophosphate or thiophosphate ester, 3 is a sulfamate, 4 is a phosphonate, 5 is a thiophosphonate, 6 is a sulfonate, 7 is a polymer, 8 is an optionally substituted oligosaccharide, 9 is a thionoester and 10 is an amide. Exemplary R⁴ moieties include (i) —O—P(O)⁻(O—(C(O))ₘ—(CH₂)ₙ—CH₃)—OH, —O—P(O)(O—(C(O))ₘ—(CH₂)ₙ—CH₃)—O—(CH₂)ₙ—CH₃ where m independently are 0 or 1 and n independently are 1, 2, 3, 4, 5, 6, 7, 8, 9, 10 or 11, (ii) —O—P(O)(SH)—OH, —O—P(O)(SH)—O⁻Na⁺, —O—P(O)(OH)—S-optionally substituted alkyl, —O—P(O)(S—(C(O))ₘ—(CH₂)ₙ—CH₃)—OH, —O—P(O)(S—(C(O))ₘ—(CH₂)ₙ—CH₃)—O—(CH₂)ₙ—CH₃ where m independently are 0 or 1 and n independently are 1, 2, 3, 4, 5, 6, 7, 8, 9, 10 or 11, (iii) —(OCH₂HC₂)ₙ—OH, —(OCH₂HC₂)ₙ—CH₃, —(OCH₂HC₂)ₙ—OR^{PR}, —(OCH₂HC₂)ₙ—SH, —(OCH₂HC₂)ₙ—SR^{PR}, —(OCH₂HC₂)ₙ—NH₂ or —(OCH₂HC₂)ₙ—NHR^{PR} where n is an integer such as an integer from about 4, 8, 12 or 20 to about 30, 40, 50 or 100, (vi) —O—S(O)(O)—NH—(C(O))ₘ—(CH₂)ₙ—X—(CH₂)ₙ—CH₃, —O—S(O)(O)—NH—(C(O))ₘ—(CH₂)ₙ—CH₃—O—S(O)(O)—NH—(CH₂)ₙ—X—CH₃, —O—S(O)(O)—NH—(CH₂)ₙ—(C(O))ₘ—X—(C(O))ₘ—(CH₂)ₘ—CH₃, —O—S(O)(O)—NH₂, —O—S(O)(O)—NH—C1-C8 optionally substituted alkyl, —O—S(O)(O)—N—(C1-C8 optionally substituted alkyl)₂, —NH—S(O)(O)—O—(CH₂)ₙ—X—CH₃, —NH—S(O)(O)—O—(C(O))ₘ—(CH₂)ₙ—X—CH₃, —NH—S(O)(O)—O—(CH₂)ₙ—(C(O))ₘ—X—CH₃, —NH—S(O)(O)—O—(CH₂)ₙ—X—(C(O))ₘ—CH₃ or —NH—S(O)(O)—O—(CH₂)ₘ— optionally substituted heterocycle, where X is —O—, —S—, —NH—, —N(C1-C8 optionally substituted alkyl)-, m independently are 0 or 1, n independently are 1, 2, 3, 4, 5, 6, 7, 8, 9, 10 or 11 and optionally substituted alkyl are each independently selected, (vii) —O—S(O)(O)—(C(O))ₘ—(CH₂)ₙ—X—(CH₂)ₙ—CH₃, —S(O)(O)—(C(O))ₘ—(CH₂)ₙ—CH₃, —O—S(O)(O)—(CH₂)ₙ—X—CH₃, —O—S(O)(O)—(CH₂)ₙ—(C(O))ₘ—X—(C(O))ₘ—(CH₂)ₘ—CH₃, —O—S(O)(O)—CH₃, —O—S(O)(O)—C1-C8 optionally substituted alkyl, —S(O)(O)—O—(CH₂)ₙ—X—CH₃, —S(O)(O)—O—(C(O))ₘ—(CH₂)ₙ—X—CH₃, —S(O)(O)—O—(CH₂)ₙ—(C(O))ₙ—X—CH₃, —S(O)(O)—O—(CH₂)ₙ—X—(C(O))ₘ—CH₃ or —S(O)(O)—O—(CH₂)ₘ-optionally substituted heterocycle, where X is —O—, —S—, —NH—, —N(C1-C8 optionally substituted alkyl)-, m independently are 0 or 1, n independently are 1, 2, 3, 4, 5, 6, 7, 8, 9, 10 or 11 and optionally substituted alkyl are each independently selected, (viii) —O—P(O)(OR^{PR})—(C(O))ₘ—(CH₂)ₙ—X—(CH₂)ₙ—CH₃, —O—P(O)(OR^{PR})—(C(O))ₘ—(CH₂)ₙ—CH₃, —O—P(O)(OR^{PR})—(CH₂)ₙ—X—CH₃, —O—P(O)(OR^{PR})—(CH₂)ₙ—(C(O))ₘ—X—(C(O))ₘ—(CH₂)ₘ—CH₃, —O—P(O)(OR^{PR})—CH₃, —O—P(O)(OR^{PR})—C1-C8 optionally substituted alkyl, —P(O)(OR^{PR})—O—(CH₂)ₙ—X—CH₃, —P(O)(OR^{PR})—O—(C(O))ₘ—(CH₂)ₙ—X—CH₃, —P(O)(OR^{PR})—O—(CH₂)ₙ—(C(O))ₘ—X—CH₃, —P(O)(OR^{PR})—O—C1-C8 optionally substituted alkyl or —P(O)(OR^{PR})—O—(CH₂)ₘ-optionally substituted heterocycle, where X is —O—, —S—, —NH—, —N(C1-C8 optionally substituted alkyl)-, m independently are 0 or 1, n independently are 1, 2, 3, 4, 5, 6, 7, 8, 9, 10 or 11, R^{PR} independently are —H or a protecting group and optionally substituted alkyl are each independently selected, (ix) —O—P(S)(OR^{PR})—(C(O))ₘ—(CH₂)ₙ—X—(CH₂)ₙ—CH₃, —O—P(S)(OR^{PR})—(C(O))ₘ—(CH₂)ₙ—CH₃, —O—P(S)(OR^{PR)—(CH}₂)ₙ—X—CH₃, —O—P(S)(OR^{PR})—(CH₂)ₙ—(C(O)ₘ—X—(C(O)ₘ—(CH₂)ₘ—CH₃, —O—P(S)(OP^{PR})—CH₃, —O—P(S)(OR^{PR})—C1-C8 optionally substituted alkyl, —P(S)(OR^{PR})—O—(CH₂)ₙ—X—CH₃, —P(S)(OR^{PR})—O—(C(O))ₘ—(CH₂)ₙ—X—CH₃, —P(S)(OR^{PR})—O—(CH₂)ₙ—(C(O))ₘ—X—CH₃, —P(S)(OR^{PR})—O—C1-C8 optionally substituted alkyl or —P(S)(OR^{PR})—O—(CH₂)ₘ-optionally substituted heterocycle, where X is —O—, —S—, —NH—, —N(C1-C8 optionally substituted alkyl)-, m independently are 0 or 1, n independently are 1, 2, 3, 4, 5, 6, 7, 8, 9, 10 or 11, R^{PR} independently are —H or a protecting group and optionally substituted alkyl are each independently selected and (x) —C(O)—NH—(C(O))ₘ—(CH₂)ₙ—X—(CH₂)ₙ—CH₃, —C(O)—NH—(C(O))ₘ—(CH₂)_{n—CH}₃, —C(O)—NH——(CH₂)ₙ—X—CH₃, —C(O)—NH—(CH₂)ₙ—(C(O))ₘ—X—(C(O))ₘ—(CH₂)ₘ—CH₃, —C(O)—NH—CH₃, —C(O)—NH—C1-C8 optionally substituted alkyl, —C(O)—NH—CH₂—CH₂—CH₃, —C(O)—NH—CH₂OR^{PR}, —C(O)—NH—CH₂—CH₂—CH₂OR^{PR}, —C(O)—NH—CH₂—CH₂—CH₂—CH₃, —C(O)—NH—CH₂—CH₂—CH₂—CH₂C(O)OR^{PR}, —C(O)—NH—CH₂—CH₂C(O)OR^{PR}, —NH—C(O)—(CH₂)ₙ—X—CH₃, —NH—C(O)—(C(O))ₘ—(CH₂)ₙ—X—CH₃, —NH—C(O)—(CH₂)ₙ—(C(O))ₘ—X—CH₃, or —NH—C(O)—(CH₂)ₘ-optionally substituted heterocycle, where X is —O—, —S—, —NH—, —N(C1-C8 optionally substituted alkyl)-, m independently are 0 or 1, n independently are 1, 2, 3, 4, 5, 6, 7, 8, 9, 10 or 11, R^{PR} independently are —H or a protecting group and optionally substituted alkyl are each independently selected.

(9) Compounds in any of the foregoing groups 1 through 56-55-54-53-52-51-50-47 and in paragraphs (1), (2), (3), (4), (5), (6), (7) and (8) in this group 57 where R³ moieties 1 through 10 in Table A are replaced with the following moieties: 1 is —O-optionally substituted alkyl, 2 is an ester (e.g., —O—C(O)—(CH₂)ₙ—CH₃, —O—C(O)—(CH₂)ₙ—NH₂, —O—C(O)—(CH₂)ₙ—NHR^{PR}, —O—C(O)—(CH₂)ₙ—CH₂(ZR^{PR}, —O—C(O)—CH(ZR^{PR})—(CH₂)ₙ—CH₃ or another ester described herein, where n is 0, 1, 2, 3, 4, 5, 6, 7 or 8, Z is —NH—, —O— or —S— and R^{PR} independently or together are —H, a protecting group or a counterion, e.g., methoxymethyl, —CH₃ or —C₂H₅), 3 is a thioester (e.g., —S—C(O)—(CH₂)ₙ—CH₃, —S—C(O)—(CH₂)ₙ—NH₂, —S—C(O)—(CH₂)ₙ—N(R^{PR})₂, —S—C(O)—(CH₂)ₙ—CH₂ZR^{PR}, —S—C(O)—CH(ZR^{PR})—(CH₂)ₙ—CH₃ or another thioester described herein, where n is 0, 1, 2, 3, 4, 5, 6, 7 or 8, Z is —NH—, —O— or —S— and R^{PR} is —H or a protecting group, e.g., —CH₃ or —C₂H₅), 4 is a carbonate (e.g., —O—C(O)—O-Optionally substituted alkyl), 5 is optionally substituted alkylamine (e.g., —NH-Optionally substituted alkyl), 6 is optionally substituted dialkylamine (e.g., —N(Optionally substituted alkyl)₂, where each optionally substituted alkyl is independently chosen), 7 is an N linked carbamate (e.g., —NH—C(O)—O-Optionally substituted alkyl or —NH—C(O)—OH), 8 is an O linked carbamate (e.g., —O—C(O)—NH₂ or —O—C(O)—NH-Optionally substituted alkyl), 9 is —O-optionally substituted monosaccharide and 10is —H.

(10) Compounds in any of the foregoing groups 1 through 56-55-54-53-52-51-50-47 and in paragraphs (1), (2), (3), (4), (5), (6), (7) and (8) in this group 57 R³ moieties 1 through 10 in Table A are replaced with the following moieties: 1 is —O-optionally substituted disaccharide, 2 is an N-linked amino acid, an N-linked amino acid ester or a salt (e.g., —NH—CH₂—C(O)OH, —NH—CH₂—C(O)OR^{PR}, —NH—CH₂—C(O)OCH₃, —NH—CHCH₃—C(O)OR^{PR} or —NH—CH₂—CH₂—C(O)OR^{PR}, where R^{PR} is —H, a counter ion or a protecting group and chiral carbon atoms are in the D-, -L or -DL configuration), 3 is an O-linked amino acid, an O-linked amino acid ester or a salt (e.g., —O—C(O)—CH₂—NHR^{PR}, —O—CH₂—NH₂, or —O—C(O)—CH₂—CH₂—NHR^{PR}, where R^{PR} is —H, a counter ion or a protecting group and chiral carbon atoms are in the D-, -L or -DL configuration), 4 is an S-linked amino acid, an S-linked amino acid ester or a salt (e.g., —S—C(O)—CH₂—NHR^{PR}, —S—CH₂—NH₂, or —S—C(O)—CH₂—CH₂—NHR^{PR}, where R^{PR} is —H, a counter ion or a protecting group and chiral carbon atoms are in the D-, -L or -DL configuration), 5 is a sulfate ester (e.g., —O—S(O)(OR^{PR})—O-Optionally substituted alkyl), 6 is —O—S(O)—O-Optionally substituted alkyl, 7 is a halogen such as —Br or —I, 8 is a halogen such as —F or —Cl, 9 is an N-linked heterocycle (e.g., N-morpholino) and 10 is a C-linked heterocycle (e.g., 2-pyrimidinyl).

(11) Compounds in any of the foregoing groups 1 through 56-55-54-53-52-51-50-47 and in paragraphs (1), (2), (3), (4), (5), (6), (7) and (8) in this group 57 where there is no double bond at the 15-16 or the 16-17 position and R³ moieties 1 through 10 in Table A are replaced with the following moieties: 1 is ═O, 2 is ═S, 3 is ═NOH, 4 is ═NOCH₃, 5 is ═NOC₂H₅, 6 is ═N—C1-C10 optionally substituted alkyl, 7 is ═NO—C1-C10 optionally substituted alkyl, 8 is ═NH, 9 is ═CH₂ and 10 is ═C-Optionally substituted alkyl. Exemplary compounds and compound genera include 3β-amino-16-oxo-17β-hydroxyandrost-5(10)-ene, 3β-amino-16-oxo-17β-hydroxyandrost-5(10)-ene, 3,16-dioxo-17β-aminoandrost-5(10)-ene, 3β-hydroxy-3α-methyl-16-oxo-17β-aminoandrost-5(10)-ene, 3β-hydroxy-3α-methyl-16-oxo-17α-aminoandrost-5(10)-ene, 3α-hydroxy-3β-ethynyl-16-oxo-17β-aminoandrost-5(10)-ene, 3β-mercapto-16-oxo-17β-hydroxyandrost-5(10)-ene, 3α-mercapto-16-oxo-17β-hydroxyandrost-5(10)-ene, 3β-amino-16-oxo-17β-hydroxyandrost-5,7-diene, 3α-amino-16-oxo-17β-hydroxyandrost-5,7-diene, 3β-amino-16-oxo-17α-hydroxyandrost-5,7-diene, 3β-hydroxy-3α-methyl-16-oxo-17β-aminoandrost-5,7-diene, 3α-hydroxy-3β-ethynyl-16-oxo-17β-aminoandrost-5,7-diene, 3β-hydroxy-16-oxo-17β-aminoandrost-5,7-diene, 3α-hydroxy-16-oxo-17β-aminoandrost-5,7-diene, 3β-hydroxy-16-oxo-17α-aminoandrost-5,7-diene, 3-amino-16-oxo-17β-hydroxyandrost-1,3-diene, 3-hydroxy-16-oxo-17β-methoxyandrost-1,3diene, 3-hydroxy-16-oxo-17α-methoxyandrost-1,3-diene, 3-amino-16-oxo-17β-hydroxy-5β-androst-1,3-diene, 3-amino-16-oxo-17-methoxy-5β-androst-1,3-diene, 3-hydroxy-16-oxo-17α-methoxy-5β-androst-1,3-diene, 3-hydroxy-16-oxo-17-methoxy-5β-androst-1,3-diene, 3-amino-16-oxo-17β-methoxy-androst-2,5(10)-diene, 3-amino-16-oxo-17α-methoxyandrost-2,5(10)-diene, 3-hydroxy-16-oxo-17β-methoxyandrost-2,5(10)-diene, 3-hydroxy-16-oxo-17α-methoxyandrost-2,5(10)-diene, 3-amino-16-oxo-17β-methoxy-5p-androst-2,5(10)-diene, 3-amino-16-oxo-17α-methoxy-5β-androst-2,5(10)-diene, 3-hydroxy-16-oxo-17β-propionoxy-5β-androst-2,5(10)-diene, 3-hydroxy-16-oxo-17α-propionoxy-5β-androst-2,5(10)-diene, 3-amino-16-oxo-17β-methoxyandrost-2,5-diene, 3-amino-16-oxo-17α-methoxyandrost-2,5-diene, 3-hydroxy-16-oxo-17β-aminoandrost-2,5-diene, 3-hydroxy-16-oxo-17α-aminoandrost-2,5-diene, 3-amino-16-oxo-17β-methoxy-5β-androst-2,5-diene, 3-amino-16-oxo-17β-hydroxy-5β-androst-2,5-diene, 3-amino-16-oxo-17α-methoxy-5β-androst-2,5-diene, 3-amino-16-oxo-17β-mercapto-5β-androst-2,5-diene, 3-amino-16-oxo-17α-mercapto-5β-androst-2,5-diene, 3-hydroxy-16-oxo-17β-propionoxy-5β-androst-2,5-diene, 3-hydroxy-16-oxo-17α-propionoxy-5β-androst-2,5-diene, 3-amino-16-oxo-17β-methoxyandrost-1,3,5-triene, 3-hydroxy-16-oxo-17α-methoxyandrost-1,3,5-triene, 3-amino-16-oxo-17β-methoxyandrost-1,3,9(11)-triene, 3-amino-16-oxo-17α-methoxyandrost-1,3,9(11)-triene, 3-hydroxy-16-oxo-17β-methoxyandrost-1,3,9(11)-triene, 3-hydroxy-16-oxo-17α-methoxyandrost-1,3,9(11)-triene, 3-amino-i 6-oxo-17β-methoxy-5β-androst-1,3,9(11)-triene, 3-amino-16-oxo-17α-methoxy -5β-androst-1,3,9(11)-triene, 3-hydroxy-16-oxo-17p-methoxy-5β-androst-1,3,9(11)-triene, 3-hydroxy-16-oxo-17α-methoxy-5β-androst-1,3,9(11)-triene, 3-amino-16-oxo-17β-methoxyandrost-1,3,5(10)-triene, 3-amino-16-oxo-17α-methoxyandrost-1,3,5(10)-triene, 3-amino-16-oxo-17β-hydroxyandrost-1,3,5(10)-triene, 3-amino-16-oxo-17α-hydroxyandrost-1,3,5(10)-triene, 3-hydroxy-16-oxo-17β-methoxyandrost-1,3,5(10)-triene, 3-hydroxy -16-oxo-17α-methoxyandrost-1,3,5(10)-triene, 3-methylamino-16-oxo-17-hydroxyandrost-1,3,5(10)-triene, 3-methylamino-16-oxo-17α-hydroxyandrost-1,3,5(10)-triene, 3-amino-16-oxo-17β-methoxyandrost-1,3,5(10),8(14)-tetraene, 3-amino-16-oxo-17α-methoxyandrost-1,3,5(10),8(1 4)-tetraene, 3-hydroxy-16-oxo-17β-methoxyandrost-1,3,5(10),8(14)-tetraene, 3-hydroxy-16-oxo-17α-methoxyandrost-1,3,5(10),8(14)-tetraene, 3-amino-16-oxo-17β-methoxyandrost-1,3,5(10),8(9)-tetraene, 3-amino-16-oxo-17α-methoxyandrost-1,3,5(10),8(9)-tetraene, 3-hydroxy-16-oxo-17β-methoxyandrost-1,3,5(10),8(9)-tetraene, 3-hydroxy-16-oxo-17α-methoxyandrost-1,3,5(10),8(9)-tetraene, 3-amino-16-oxo-17β-hydroxyandrost-1,3,5(10),6-tetraene, 3,17β-dihydroxy-16-oxoandrost-1,3,5(10),6-tetraene, 3-amino-16-oxo-17β-methoxyandrost-1,3,5(10),7-tetraene, and an analog of any of these compounds wherein (i) the 16-position (R³) is substituted with ═O, ═S, ═CH₂, ═CHCH₃, ═CHCH₂OH, ═CH—C1-C8 optionally substituted alkyl, ═NOH, ═NO—CH₃, ═NO—C1-C8 optionally substituted alkyl, ═N—CH₃, ═N—C1-C8 optionally substituted alkyl or another double bonded moiety described herein, and/or (ii) ═S, ═CH₂, ═CHCH₃, ═CHCH₂OH, ═CH—C1-C8 optionally substituted alkyl, ═NOH, ═NO—CH₃ or another double bonded moiety described herein is present at the 17-position (R⁴) or where two independently selected R⁴ moieties are present at the 17-position, and/or (iii) the 3-position (R¹) is substituted with one or two independently selected substituents such as —F, —Cl, —Br, —I, —OH, ═O, —SH, ═S, ═CH₂, —C1-C10 optionally substituted alkyl such as methyl, ethynyl or 1-propynyl, -heterocycle, —(CH₂)-heterocycle, a polymer, or-one or two other independently selected R¹ moieties described herein, where the substituent(s) is in the α-configuration or the β-configuration, and/or (iv) the 2-position (R⁹) is substituted with one or two independently selected substituents such as —F, —Cl, —Br, —I, —OH, ═O, ═S, ═CH₂, C1-C10 optionally substituted alkyl such as methyl, ethynyl or 1-propynyl, C1-C10 alkoxy such as methoxy or ethoxy, -heterocycle, —(CH₂)-heterocycle, or a polymer where, when no double bond is present at the 2-poistion, the substituent(s) is in the α-configuration or the β-configuration, and/or (v) R^{10G} at the 9-position, when present, is —F, —Cl, —Br, —I, —OH, C1-C10 optionally substituted alkyl such as methyl, ethyl, ethynyl or 1-propynyl or cyclopropyl with the 11-position or another R¹⁰ or R^{10G} moiety described herein, and/or (vi) the 7-position (R²) is substituted with one or two independently selected substituents such as —OH, ═O, ═S, ═CH₂, —NH₂, ═N—C1-C10 optionally substituted alkyl, ═CH—C1-C10 optionally substituted alkyl, —NH—C1-C10 optionally substituted alkyl such as methyl, hydroxymethyl, ethyl, hydroxyethyl, propyl or another optionally substituted alkyl described herein, —N(C1-C10 optionally substituted alkyl)₂, —C1-C10 optionally substituted alkyl such as methyl, ethynyl, 1-propynyl or another optionally substituted alkyl described herein, -heterocycle, —(CH₂)-heterocycle, a polymer or one or two other substituents described elsewhere herein, where, when no double bond is present at the 7-poistion, the substituent(s) is in the α-configuration or the β-configuration, and/or (vii) the 6-position (R^{10C}) is substituted with a substituent described herein such as sulfate, phosphate, an ester, an ether, a thioester, a thioether, a monosaccharide, an oligosaccharide, ethylene ketal (—O—CH₂CH₂—O—), a polymer, a carbonate, a carbamate, —F, —Cl, —Br, —I, —OH, —OR^{PR}, —SH, —SR^{PR}, —NH₂, —NHR^{PR}, —C(O)—OR^{PR}, —NHCH₂—C(O)—OR^{PR}, —NHCH₂CH₂—C(O)—OR^{PR}, —NHC(O)—CH₃, —NHC(O)—C₂H₅, —NHC(O)—OCH₃, —NHC(O)—OC₂H₅, —NHC(O)—OC₃H₇, —OC(O)—NHR^{PR}, —OC(O)—NHCH₃, —OC(O)—NHC₂H₅, —OC(O)—NHC₃H₇, ═O, ═S, ═CH₂, ═CH—C1-C10 optionally substituted alkyl, —C1-C10 optionally substituted alkyl, ═N—C1-C10 optionally substituted alkyl, ═N—O—C1-C10 optionally substituted alkyl, —NH—C1-C10 optionally substituted alkyl, —N(C1-C10 optionally substituted alkyl)₂, C1-C10 optionally substituted alkyl, -heterocycle, —(CH₂)-heterocycle, where each optionally substituted alkyl is one or two independently selected optionally substituted alkyl moieties described herein such as methyl, ethynyl, 1-propynyl or another optionally substituted alkyl described herein, where, when no double bond is present at the 6-poistion, the substituent is in the α-configuration or the β-configuration, and/or (viii) the 11-position (R⁸) is substituted with a substituent described herein such as sulfate, phosphate, an ester, an ether, a thioester, a thioether, a monosaccharide, —O—, —S—, —NH—, —N(CH₃)—, —N(C₂H₅)—, —N(C₃H₇)—, ═N— or is substituted with one or two independently selected R¹⁰ substituents such as —F, —Cl, —Br, —I, —OH, ═O, —SH, ═S, ═CH₂, C1-C10 optionally substituted alkyl such as methyl, ethynyl or 1-propynyl, -heterocycle, —(CH₂)-heterocycle, a polymer or another moiety described herein, where, when no double bond is present at the 11-poistion, the substituents are in the α-configuration or the β-configuration, e.g., R⁸ is —CH(α-C1-C10 optionally substituted alkyl)-, —CH(β-C1-C10 optionally substituted alkyl)-, —CH(β-F)—, —CH(α-F)—, —CF₂——CH(β-OH)—, —CH(α-OH)—, —C(O)—, —CH(β-SH)—, —CH(α-SH)—, —CH(β-NH₂)—, —CH(α-NH₂)—, —CH(β-NHCH₃)—, —CH(α-NHCH₃)—, —CH(β-N(CH₃)₂)—, —CH(α-N(CH₃)₂)—, —CH(β-NHC₂H₅)—, —CH(α-NHC₂H₅)—, —CH(α-heterocycle)-, —CH(β-heterocycle)-, —CH(α-polymer)-, —CH(β-polymer)-, —CH(α-ether)-, —CH(β-ether)-, —CH(α-thioether)-, —CH(β-thioether)-. Analogs of any of these compounds include compounds where substitutions described at two or three of (i), (ii), (iii), (iv), (v), (vi), (vii) and (viii) are present, e.g., substitutions as described at (i) and (ii), (i) and (iii), (i) and (iv), (i) and (vi), (i) and (vii), (i) and (viii), (i), (ii) and (iii), (i), (ii) and (vi), (i), (ii) and (v), (i), (ii) and (vi), (i), (ii) and (vii), (i), (ii) and (viii), (ii) and (iii), (ii) and (iv), (ii) and (v), (ii) and (vi), (ii) and (vii), (ii) and (viii), (i), (ii) and (iii), (i), (ii) and (iv), (i), (ii) and (v), (i), (ii) and (vi), (i), (ii) and (vii), (i), (ii) and (viii), (iii) and (iv), (iii) and (v), (iii) and (vi), (iii) and (vii), (iii) and (viii), (i), (iii) and (iv), (i), (iii) and (v), (i), (iii) and (vi), (i), (iii) and (vii), (i), (iii) and (viii), (iv) and (v), (iv) and (vi), (iv) and (vii), (iv) and (viii), (i), (iv) and (v), (i), (iv) and (vi), (i), (iv) and (vii), (i), (iv) and (viii), (v) and (vi), (v) and (vii), (v) and (viii), (i), (v) and (vi), (i), (v) and (vii), (i), (v) and (viii), (vi) and (vii), (vi) and (viii), (i), (vi) and (vii), (i), (vi) and (viii), (ii), (iii) and (iv), (ii), (iii) and (v), (ii), (iii) and (vi), (ii), (iii) and (vii) or at (ii), (iii) and (viii) are present.

(12) Compounds in any of the foregoing groups 1 through 56-55-54-53-52-51-50-47 and in paragraphs (1), (2), (3), (4), (5), (6), (7), (8), (9), (10) and (11) in this group 57 where R² moieties 1 through 10 in Table A are replaced with the following moieties: 1 is —O-optionally substituted alkyl, 2 is an ester (e.g., —O—C(O)—(CH₂)ₙ—CH₃, —O—C(O)—(CH₂)ₙ—NH₂, —O—C(O)—(CH₂)ₙ—NHR^{PR}, —O—C(O)—(CH₂)ₙ—CH₂ZR^{PR}, —O—C(O)—CH(ZR^{PR})—(CH₂)ₙ—CH₃ or another ester described herein, where n is 0, 1, 2, 3, 4, 5, 6, 7 or 8, Z is —NH—, —O— or —S— and R^{PR} is —H or a protecting group, e.g., methoxymethyl, —CH₃ or —C₂H₅), 3 is a thioester (e.g., —S—C(O)—(CH₂)ₙ—CH₃, —S—C(O)—(CH₂)ₙ—NH₂, —S—C(O)—(CH₂)ₙ—N(R^{PR})₂, —S—C(O)—(CH₂)ₙ—CH₂ZR^{PR}, —S—C(O)—CH(ZR^{PR})—(CH₂)ₙ—CH₃ or another thioester described herein, where n is 0, 1, 2, 3, 4, 5, 6, 7 or 8, Z is —NH—, —O— or —S— and R^{PR} independently or together are —H, a protecting group or a counterion, e.g., —CH₃ or —C₂H₅), 4 is a carbonate (e.g., —O—C(O)—O-Optionally substituted alkyl), 5 is optionally substituted alkylamine (e.g., —NH-Optionally substituted alkyl), 6 is optionally substituted dialkylamine (e.g., —N(Optionally substituted alkyl)₂, where each optionally substituted alkyl is independently chosen), 7 is an N linked carbamate (e.g., —NH—C(O)—O-Optionally substituted alkyl or —NH—C(O)—OH), 8 is an O linked carbamate (e.g., —O—C(O)—NH₂ or —O—C(O)—NH-Optionally substituted alkyl), 9 is —O-optionally substituted monosaccharide and 10 is —H.

(13) Compounds in any of the foregoing groups 1 through 56-55-54-53-52-51-50-47 and in paragraphs (1), (2), (3), (4), (5), (6), (7), (8), (9), (10) and (11) in this group 57 where R² moieties 1 through 10 in Table A are replaced with the following moieties: 1 is —O-optionally substituted disaccharide, 2 is an N-linked amino acid, an N-linked amino acid ester or a salt (e.g., —NH—CH₂—-C(O)OH, —NH—CH₂—C(O)OR^{PR}, —NH—CHCH₃—C(O)OR^{PR} or —NH—CH₂-CH₂—C(O)OR^{PR}, where R^{PR} is —H, a counter ion or a protecting group and chiral carbon atoms are in the D-, -L or -DL configuration), 3 is an O-linked amino acid, an O-linked amino acid ester or a salt (e.g., —O—C(O)—CH₂—NHR^{PR}, —O—CH₂—NH₂, or —O—C(O)—CH₂—C, where R^{PR} is —H, a counter ion or a protecting group and chiral carbon atoms are in the D-, -L or -DL configuration), 4 is an S-linked amino acid, an S-linked amino acid ester or a salt (e.g., —S—C(O)—CH₂—NHR^{PR}, —S—CH₂—NH₂, or —S—C(O)—CH₂—CH₂—NHR^{PR}, where R^{PR} is —H, a counter ion or a protecting group and chiral carbon atoms are in the D-, -L or -DL configuration), 5 is a sulfate ester (e.g., —O—S(O)(OR^{PR})—O-Optionally substituted alkyl), 6 is —O—S(O)—O-Optionally substituted alkyl, 7 is a halogen such as —Br or —I, 8 is a halogen such as —F or —Cl, 9 is an N-linked heterocycle (e.g., N-morpholino) and 10 is a C-linked heterocycle (e.g., 2-pyrimidinyl).

(14) Compounds in any of the foregoing groups and in (1), (2), (3), (4), (5), (6), (7), (8) and (9) in this group where there is no double bond at the 6-7 or the 7-8 position and R² moieties 1 through 10 in Table A are replaced with the following moieties: 1 is ═O, 2 is ═S, 3 is ═NOH, 4 is ═NOCH₃, 5 is ═NOC₂H₅, 6 is ═N—C1-C10 optionally substituted alkyl, 7 is ═NO—C1-C10 optionally substituted alkyl, 8 is ═NH, 9 is ═CH₂ and 10 is ═CH-optionally substituted alkyl.

(15) Compounds in any of the foregoing groups and in (1), (2), (3), (4), (5), (6), (7), (8), (9), (10), (11), (12), (13) and (14) in this group where (i) no double bond is present at the 10-position and R⁶ is a moiety other than —CH₃. Exemplary R⁶ moieties are —H, —F, —Cl, —Br, —I, —OH, —OR^{PR}, —SH, —SR^{PR}, —NH₂, —NHR^{PR}, —CHO, —CH₂OH, optionally substituted alkyl, ether, thioether, —NH-optionally substituted alkyl, ethynyl, 1-propynyl, vinyl, allyl, —O—C(O)—O-optionally substituted alkyl, —O—C(O)-optionally substituted alkyl, —O—C(O)—S-optionally substituted alkyl, —O-optionally substituted monosaccharide and a polymer.

As is apparent from the description of F1Cs, when no double bond is present at the carbon atoms at the 1-, 4- or 6-positions, R^{10A}, R^{10B}, R^{10C} and R^{10D} respectively can be in the α,α,α,α,α,α, α,β,α,α,β,ααβ,α,α, β,α,α,α, α,α,β,β, α, β,α,β, β,α,α,β, α,β,β, α, β,α,β,α, β,β, α,α, β,β, α,α, αβ,β,β, β,α,β,β, β,β,α,β, β,β,β, α or β,β,β,β configurations. As used here, reference to, e.g., R^{10A}, R^{10B}, R^{10C} and R^{10D} respectively being in the α,β,α,β configurations means that R^{10A} is in the α-configuration, R^{10B} is in the β-configuration, R^{10C} is in the α-configuration and R^{10D} is in the β-configuration. Similarly, when R^{10A}, R^{10B}, R^{10C} and R^{10D} respectively are in the α,α,β,α configurations, R^{10A} is in the α-configuration, R^{10B} is in the α-configuration, R^{10C} is in the β-configuration and R^{10D} is in the α-configuration.

Thus, when a double bond is present at one or more of the 1-, 4- or 6-positions, the corresponding R^{10A}, R^{10B} or R^{10C} moiety will not be in a specified configuration. Thus, this group contains compounds having structures where (1) a double bond is present at the 1-position, R^{10B}, R^{10C} and R^{10D} respectively are in the α,α,α, α,α,β, αβ,α, β,α,α, α,β,β, β,α,β, β,β,α or β, β, β configurations and R^{10A} is present at the 1-position with no specified configuration, (2) a double bond is present at the 4-position, R^{10A}, R^{10C} and R^{10D} respectively are in the α,α,α, α,α,β, α,β,α, β,α,α, α,β,β, β,α,β, β,β, α or β,β,β configurations and R^{10B} is present at the 4-position with no specified configuration, (3) a double bond is present at the 6-position, R^{10A}, R^{10B} and R^{10D} respectively are in the α,α,α, α,α,β, α,β,α, β, α, α, α,β,β, β,α,β, β,β,α or β,β,β configurations, and R^{10C} is present at the 6-position with no specified configuration, (4) a double bond is present at the 1-position and at the 4-position, R^{10C} and R^{10D} respectively are in the α,α, α,β, β,α, or β,β configurations and R^{10A} and R^{10B} are present at the 1- and 4-positions with no specified configuration, (5) a double bond is present at the 1-position and at the 6-position, R^{10B} and R^{10D} respectively are in the α,α, α,β, β,α, or β, β, configurations and R^{10A} and R^{10C} are present at the 1- and 6-positions with no specified configuration, (6) a double bond is present at the 4-position and at the 6-position, R^{10A} and R^{10D} respectively are in the α,α, α,β, β,α, or β,β configurations and R^{10B} and R^{10C} are present at the 4- and 6-positions with no specified configuration, (7) a double bond is present at the 1-, 4- and 6-position, R^{10D} is in the α-configuration or the β-configuration, while R^{10A}, R^{10B} and R^{10C} are present at the 1-, 4- and 6-positions with no specified configuration and (8) one, two or more additional double bonds are optionally also present at the 8-, 9-, 11-, 14-, 15- or 16-positions for any compound or genus of compounds described in (1), (2), (3), (4), (5), (6) or (7).

As is apparent from the F1Cs described in groups 1 through 57, compound groups 14 through 57 contain a number of defined subgroups, e.g., group 14-3 is a subgroup as described for group 14 compounds where R¹, R², R³ and R⁴ can be in the configurations described in group 14, e.g., α,β,α,β, α,α,α,β, β,β,β,β, β,β,β,α, or β, β,α,α respectively. Similarly, group 49 includes subgroups such as 49-18-17-14-3, 49-18-17-14-4, 49-18-17-14-5, 49-18-17-14-5A, 49-18-17-14-6, 49-18-17-14-7 and 49-18-17-14-9, which are subgroups where R⁹ is substituted, e.g., R⁹ is —O— or a moiety described in group 18, and such subgroups, although not specifically named or described, are expressly included in group 49. The F1C therefore include all possible subgroups in each group, regardless of whether each subgroup is specifically named or described in a given group or not. For example, groups such as 22, 23, 26, 26B, 26C, 26D and 26E, all include subgroups analogous to those described in group 26A and additional subgroups that are not expressly described, e.g., subgroups such as 26-18-1, 26-18-2, 26-18-3, 26-18-4,26-18-5, 26-18-5A, 26-18-6, 26-18-14-1, 26-18-14-2, 26-18-14-3, 26-18-14-4, 26-18-14-5, 26-18-14-5A and 26-18-14-6 are not described expressly in group 26 above but are included in group 26. Similarly, groups 29, 30, 33, 33B, 33C, 33D and 33E, all include subgroups analogous to those described in group 33A, while groups 36, 37, 40B, 40C, 40D, 40E and 41 all include subgroups analogous to those described in group 40A and groups 47B, 47C, 47D, 47E and 48 all include subgroups analogous to those described in group 47A. Thus, subgroups such as 33-18-3 and 33-18-14-3, which are not described expressly in group 33 above, are included in group 33.

The F1Cs include compounds in groups 1 through 57 where R^{10F} and/or R^{10H} is a moiety other than hydrogen, e.g., a halogen, an ether, a thioether, a polymer or optionally substituted alkyl such as —F, —Cl, —Br, —I, —CH₃, —OCH₃, —SCH₃, —OH, —OR^{PR}, —SH, —SR^{PR}, —NH₂ or —NHR^{PR} where R^{PR} independently are —H or a protecting group. Thus, for any of the compounds or genera of compounds in groups 1 through 57, R^{10F} can be —F, —Cl, —CH₃ or —OH in the α- or β-configuration. Similarly, in groups 1 through 57, R^{10H} can be —F, —NH₂, —OH, —SH, —CH₃, —C₂H₅ or —CH₂OH in the α- or β-configuration or an epoxide or cyclopropyl ring with R⁷ where the ring bonds are in the α- or β-configuration.

The F1Cs include analogs of compounds in groups 1 through 57 where R¹¹ is a moiety such as —O—, ═N—, —NH—, —NCH₃—, —NC₂H₅—, —S—, —S(O)(O)— or another moiety disclosed herein within the scope of the R¹¹ definition. As is apparent from the F1C structures, when R¹¹ is a moiety such as —O— or —S—, a double bond at the 3-4 or 4-5 position will not be present. Exemplary F1Cs where R¹¹ is one of these moieties includes 3β, 17β-dihydroxy-3α-C1-8 optionally substituted alkyl-4-aza-androst-1,5-diene, 3β, 17β-dihydroxy-4-aza-androst-1,5-diene, 3α,17β-dihydroxy-3β-C1-8 optionally substituted alkyl-4-aza-androst-1,5-diene, 3α,17β-dihydroxy-4-aza-androst-1,5-diene, 3β-hydroxy-3α-C1-8 optionally substituted alkyl-4-aza-17-thioxoandrost-1,5-diene, 3β-hydroxy-4-aza-17-thioxoandrost-1,5-diene, 3α-hydroxy-3α-C1-8 optionally substituted alkyl-4-aza-17-thioxoandrost-1,5-diene, 3α-hydroxy-4-aza-17-thioxoandrost-1,5-diene, 3β,17β-dihydroxy-3α-C1-8 optionally substituted alkyl-2,4-dioxa-androst-1,5-diene, 3β,17β-dihydroxy-2,4-dioxa-androst-1,5-diene, 3α,17β-dihydroxy-3β-C1-8 optionally substituted alkyl-2,4-dioxa-androst-1,5-diene, 3α,17β-dihydroxy-2,4-dioxa-androst-1,5-diene, 3β,17β-dihydroxy-3α-C1-8 optionally substituted alkyl-4-thia-androst-1,5-diene, 3β,17β-dihydroxy-4-thia-androst-1,5-diene, 3α,17β-dihydroxy-3β-C1-8 optionally substituted alkyl-4-thia-androst-1,5-diene, 3α,17β-dihydroxy-4-thia-androst-1,5-diene, 3β,17β-dihydroxy-3α-C1-8 optionally substituted alkyl-4-oxa-androst-1,5-diene, 3β,17β-dihydroxy-4-oxa-androst-1,5-diene, 3α,17β-dihydroxy-3β-C1-8 optionally substituted alkyl-4-oxa-androst-1,5-diene, 3α,17β-dihydroxy-4-oxa-androst-1,5-diene, 3β,17β-dihydroxy-3α-C1-8 optionally substituted alkyl-4-aza-androstane, 3β,17β-dihydroxy-4-aza-androstane, 3α,17β-dihydroxy-3β-C1-8 optionally substituted alkyl-4-aza-androstane, 3α,17β-dihydroxy-4-aza-androstane, 3β,17β-dihydroxy-3α-C1-8 optionally substituted alkyl-4-aza-5β-androstane, 3β,17β-dihydroxy-4-aza-5β-androstane, 3α,17β-dihydroxy-3β-C1-8 optionally substituted alkyl-4-aza-5β-androstane, 3α,17β-dihydroxy-4-aza-5β-androstane and analogs of any of these compounds where independently selected —OH, —NH₂, —NHCH₃, —SH, —F, —Cl, —Br, —I, C1-8 optionally substituted alkyl or another oxygen-, nitrogen- or sulfur-linked moiety is present at 1, 2 or 3 of the 2-position, the 6-position, the 7-position, the 12-position and/or the 16-position, any of which are in the α- or β-configuration when no double bond is present at the substituted position, or analogs wherein one or more of these positions is substituted with a double bonded moiety such as ═O, ═S, ═NOH, ═N—C1-8 optionally substituted alkyl, or ═CH—C1-8 optionally substituted alkyl, or a 19-nor, D ring homo, 1-ene, 2-ene, 3-ene, 4-ene, 5-ene (i.e., 5(6)-ene), 5(10)-ene, 9(11)-ene, 11-ene, 12-ene, 15-ene, 16-ene 1,4-diene, 1,15-diene, 1,16-diene, 3,5-diene, 5,7-diene or aromatic A ring analog of any of these compounds or analogs. Other exemplary analogs include compounds and genera of compounds of any of these compounds where the moiety at the 3- and/or 17-position is replaced with independently selected moieties as described herein such as ═O, ═S, ═NOH, —SH, —NH₂, —NHCH₃, —NHC₂H₅, —N(CH₃)₂, —N(C₂H₅)₂, —NH(C1-8 optionally substituted alkyl), —N(C1-8 optionally substituted alkyl)₂, —C(O)—CH₃, —O—C(O)—CH₃, —O—C(O)—CF₃, —C(S)—CH₃, —S—C(O)—CH₃, —C(O)—CH₂Cl, —C(O)—CH₂OH, ester such as a C2-8 ester, thioester such as a C2-8 thioester, ether such as a C1-8 ether, thioether such as C1-8 thioether, a carbamate such as a C1-8 carbamate, a carbonate such as a C1-8 carbonate, an optionally substituted monosaccharide or a polymer.

The formula 1 compounds may contain 0, 1, 2, 3, 4 or 5 carbon-carbon or carbon-nitrogen double bonds within the fused four-ring system, such that the compound is unsaturated. Classes of formula 1 compounds include, androstanes (or 5α-androstanes), 5α-androstanes, 1-ene, 2-ene, 3-ene, 4-ene, 5(6)-ene (or a “5-ene”), 5(10)-ene, 6-ene, 7-ene, 8(9)-ene, 8(14)-ene, 9(10)-ene, 9(11)-ene, 11-ene, 12-ene, 13(17)-ene, 14-ene, 15-ene, 16-ene, 1,3-diene, 1,4-diene, 1,5-diene, 1,5(10)-diene, 1,6-diene, 1,7-diene, 1,8(9)-diene, 1,8(14)-diene, 1,9(11)-diene, 1,11-diene, 1,12-diene, 1,13(17)-diene, 1,15-diene, 1,16-diene, 2,4-diene, 2,5-diene, 2,5(10)-diene, 2,6-diene, 2,7-diene, 2,8(9)-diene, 2,8(14)-diene, 2,11-diene, 2,12-diene, 2,13(17)-diene, 2,14-diene, 2,15-diene, 2,16-diene, 3,5-diene, 3,6-diene, 3,7-diene, 3,8(9)-diene, 3,8(14)-diene, 3,9(10)-diene, 3,9(11)-diene, 3,11-diene, 3,12-diene, 3,13(17)-diene, 3,14-diene, 3,15-diene, 3,16-diene, 4,6-diene, 4,7-diene, 4,8(9)-diene, 4,8(14)-diene, 4,9(10)-diene, 4,9(11)-diene, 4,11-diene, 4,12-diene, 4,13(17)-diene, 4,14-diene, 4,15-diene, 4,16-diene, 5(6),15-diene (or a “5,15-diene”), 5,7-diene, 5,8(9)-diene, 5,8(14)-diene, 5,9(11)-diene, 5,11-diene, 5,12-diene, 5,13(17)-diene, 5,14-diene, 5,15-diene, 5,16-diene, 5(10),7-diene, 5(10),8(9)-diene, 5(10),8(14)-diene, 5,9(11)-diene, 5(10),12-diene, 5(10),13(17)-diene, 5(10),14-diene, 5(10),15-diene, 5(10),16-diene, 6,9(11)-diene, 6,9(14)-diene, 6,10-diene, 6,11-diene, 6,13(17)-diene, 6,14-diene, 6,15-diene, 6,16-diene, 7,9(10)-diene, 7,9(11)-diene, 7,12-diene, 7,13(17)-diene, 7,14-diene, 7,15-diene, 7,16-diene, 8(9), 11-diene, 8(9),12-diene, 8(9),13(17)-diene, 8(9),14-diene, 8(9),15-diene, 8(9),16-diene, 8(14),9-diene, 8(14), 11-diene, 8(14),12-diene, 8(14),13(17)-diene, 8(14),15-diene, 8(14),16-diene, 9(10), 11-diene, 9(10),12-diene, 9(10),13(17)-diene, 9(10),14-diene, 9(10),15-diene, 9(10),16-diene, 9(11),13-diene, 9(11),13(17)-diene, 9(11),14-diene, 9(11),15-diene, 9(11),16-diene, 11,13(17)-diene, 11,14-diene, 11,15-diene, 11,16-diene, 12,14-diene, 12,15-diene, 12,16-diene, 13(17),14-diene, 13(17),15-diene, 14,16-diene, 1,3,5-triene, 1,3,5(10)-triene, 1,3,6-triene, 1,3,7-triene, 1,3,8-triene, 1,3,8(14)-triene, 1,3,9-triene, 1,3,9(11)-triene, 1,3,12-triene, 1,3,13(17)-triene, 1,3,14-triene, 1,3,15-triene, 1,3,16-triene, 1,4,6-triene, 1,4,7-triene, 1,4,8-triene, 1,4,8(14)-triene, 1,4,9-triene, 1,4,11-triene, 1,4,9(11)-triene, 1,4,12-triene, 1,4,13(17)-triene, 1,4,14-triene, 1,4,15-triene, 1,4,16-triene, 1,5,7-triene, 1,5,8-triene, 1,5,8(14)-triene, 1,5,9-triene, 1,5,9(11)-triene, 1,5,11-triene, 1,5,12-triene, 1,5,13(17)-triene, 1,5,14-triene, 1,5,15-triene, 1,5,16-triene, 1,5(10),6-triene, 1,5(10),7-triene, 1,5(10),8-triene, 1,5(10),8(14)-triene, 1,5(10),9(11)-triene, 1,5(10),12-triene, 1,5(10),13(17)-triene, 1,5(10),14-triene, 1,5(10),15-triene, 1,5(10),16-triene, 1,6,8-triene, 1,6,8(14)-triene, 1,6,9-triene, 1,6,9(11)-triene, 1,6,11-triene, 1,6,12-triene, 1,6,13(17)-triene, 1,6,14-triene, 1,6,15-triene, 1,6,16-triene, 1,7,9-triene, 1,7,9(11)-triene, 1,7,11-triene, 1,7,12-triene, 1,7,13(17)-triene, 1,7,14-triene, 1,7,15-triene, 1,7,16-triene, 2,4,6-triene, 2,5,6-triene, 2,5(10),6-triene, 2,4,7-triene, 2,5,7-triene, 2,5(10),7-triene, 2,4,8-triene, 2,5,8-triene, 2,5(10),8-triene, 2,4,8(14)-triene, 2,5,8(14)-triene, 2,5(10),8(14)-triene, 2,4,9-triene, 2,4,9(11)-triene, 2,5,9(11)-triene, 2,5(10),9(11)-triene, 2,4,11-triene, 2,5,11-triene, 2,5(10),11-triene, 2,4,12-triene, 2,5,12-triene, 2,5(10),12-triene, 2,4,14-triene, 2,5,14-triene, 2,5(10),14-triene, 2,4,15-triene 2,5,15-triene, 2,5(10),15-triene, 2,4,16-triene, 2,5,16-triene, 2,5(10),16-triene, 2,6,8-triene, 2,6,8(14)-triene, 2,6,9-triene, 2,6,9(11)-triene, 2,6,12-triene, 2,6,13(17)-triene, 2,6,14-triene, 2,6,15-triene, 2,6,16-triene, 2,7,9-triene, 2,7,9(11)-triene, 2,7,12-triene, 2,7,13(17)-triene, 2,7,14-triene, 2,7,15-triene, 2,7,16-triene, 3,5,9-triene, 3,5,11-triene, 3,5,12-triene, 3,5,13-triene, 3,5,14-triene, 3,5,15-triene, 3,5,16-triene, 3,6,8-triene, 3,6,8(14)-triene, 3,6,9-triene, 3,6,9(11)-triene, 3,6,11-triene, 3,6,12-triene, 3,6,13(17)-triene, 3,6,14-triene, 3,6,15-triene, 3,6,16-triene, 3,7,9-triene, 3,7,11-triene, 3,7,12-triene, 3,7,13(17)-triene, 3,7,14-triene, 3,7,15-triene, 3,7,16-triene,3,8,11-triene, 3,8,12-triene, 3,8,13(17)-triene, 3,8,14-triene, 3,8,15-triene, 3,8,16-triene, 3,8(14), 11-triene, 3,8(14),12-triene, 3,8(14),13(17)-triene, 3,8(14),15-triene, 3,8(14),16-triene, 3,9,11-triene, 3,9,12-triene, 3,9,13(17)-triene, 3,9,14-triene, 3,9,15-triene, 3,9,16-triene, 3,9(11),12-triene, 3,9(11),13(17)-triene, 3,9(11),14-triene, 3,9(11),15-triene, 3,9(11),16-triene, 3,11,13(17)-triene, 3,11,14-triene, 3,11,1 5-triene, 3,11,16-triene, 3,12,14-triene, 3,12,15-triene, 3,12,16-triene, 3,13(17),14-triene, 3,13(17),15-triene, 3,14,16-triene, 4,6,8-triene, 4,6,8(14)-triene, 4,6,9-triene, 4,6,9(11)-triene, 4,6,11-triene, 4,6,12-triene, 4,6,13(17)-triene, 4,6,14-triene, 4,6,15-triene, 4,6,16-triene, 4,7,9-triene, 4,7,11-triene, 4,7,12-triene, 4,7,13(17)-triene, 4,7,14-triene, 4,7,15-triene, 4,7,16-triene, 4,8,9-triene, 4,8,9(11)-triene, 4,8,11-triene, 4,8,12-triene, 4,8,13(17)-triene, 4,8,14-triene, 4,8,15-triene, 4,8,16-triene, 4,8(14),9-triene, 4,8(14),9(11)-triene, 4,8(14), 11-triene, 4,8(14),13(17)-triene, 4,8(14),15-triene, 4,8(14),16-triene, 4,9,11-triene, 4,9,13(17)-triene, 4,9,14-triene, 4,9,15-triene, 4,9,16-triene, 4,9(11),12-triene, 4,9(11),13(17)-triene, 4,9(11),14-triene, 4,9(11),15-triene, 4,9(11),16-triene, 4,9(11),12-triene, triene, 4,11,14-triene, 4,11,15-triene, 4,11,16-triene, 4,12,14-triene, 4,12,15-triene, 4,12,16-triene, 4,13(17),14-triene, 4,13(17),15-triene, 4,14,16-triene, 5,7,9-triene, 5,7,9(11)-triene, 5,7,12-triene, 5,7,13(17)-triene, 5,7,14-triene, 5,7,15-triene, 5,7,16-triene, 5,8,11-triene, 5,8,12-triene, 5,8,13(17)-triene, 5,8,14-triene, 5,8,15-triene, 5,8,16-triene, 5,8(14),9-triene, 5,8(14),9(11)-triene, 5,8(14),12-triene, 5,8(14),13(17)-triene, 5,8(14),15-triene, 5,8(14),16-triene, 5,9,11-triene, 5,9,12-triene, 5,9,13(17)-triene, 5,9,14-triene, 5,9,15-triene, 5,9,16-triene, 5,9(11),12-triene, 5,9(11),13(17)-triene, 5,9(11),14-triene, 5,9(11),15-triene, 5,9(11),16-triene, 5,11,13(17)-triene, 5,11,14-triene, 5,11,15-triene, 5,11,16-triene, 5,12,14-triene, 5,12,15-triene, 5,12,16-triene, 5,13(17),14-triene, 5,13(17),15-triene, 5,14,16-triene, 6,8,11-triene, 6,8,12-triene, 6,8,13(17)-triene, 6,8,14-triene, 6,8,15-triene, 6,8,16-triene, 6,8(14),9-triene, 6,8(14),9(11)-triene, 6,8(14),11-triene, 6,8(14),12-triene, 6,8(14),13(17)-triene, 6,8(14),16-triene, 6,9,11-triene, 6,9,12-triene, 6,9,13(17)-triene, 6,9,14-triene, 6,9,15-triene, 6,9,16-triene, 6,9(11),12-triene, 6,9(11),13(17)-triene, 6,9(11),14-triene, 6,9(11),15-triene, 6,9(11),16-triene, 6,11,13(17)-triene, 6,11,14-triene, 6,11,15-triene, 6,11,16-triene, 6,12,14-triene, 6,12,15-triene, 6,12,16-triene, 6,13(17),14-triene, 6,13(17),15-triene, 6,14,16-triene, 7,9,11-triene, 7,9,12-triene, 7,9,13(17)-triene, 7,9,14-triene, 7,9,15-triene, 7,9,16-triene, 7,9(11),12-triene, 7,9(11),13(17)-triene, 7,9(11),14-triene, 7,9(11),15-triene, 7,9(11),16-triene, 7,12,14-triene, 7,12,15-triene, 7,12,16-triene, 7,13(17),14-triene, 7,13(17),15-triene, 7,14,16-triene, 8,11,13(17)-triene, 8,11 ,14-triene, 8,11,15-triene, 8,11,16-triene, 8,12,14-triene, 8,12,15-triene, 8,12,16-triene, 8,13(17),14-triene, 8,13(17),15-triene, 8,14,16-triene, 8(14),9,11-triene, 8(14),9,12-triene, 8(14),9,13(17)-triene, 8(14),9,15-triene, 8(14),9,16-triene, 8(14),9(11),12-triene, 8(14)9(11),13(17)-triene, 8(14),9(11),15-triene, 8(14),9(11),16-triene, 9,11,13(17)-triene, 9,11,14-triene, 9,11,15-triene, 9,11,16-triene, 9(11),13(17),14-triene, 9(11),13(17),15-triene, 11,13(17),14-triene, 11,13(17),15-triene, 12,14,16-triene, 1,3,5(10),6-tetraene, 1,3,5(10),7-tetraene, 1,3,5(10),8(9)-tetraene, 1,3,5(10),8(14)-tetraene, 1,3,5(10),9(11)-tetraene, 1,3,5(10),11-tetraene, 1,3,5(10),12-tetraene, 1,3,5(10),13(17)-tetraene, 1,3,5(10),14-tetraene, 1,3,5(10),15-tetraene, 1,3,5(10),16-tetraene, 1,3,5,7-tetraene, 1,3,5,8-tetraene, 1,3,5,8(14)-tetraene, 1,3,5,9-tetraene, 1,3,5,9(11)-tetraene, 1,3,5,12-tetraene, 1,3,5,13(17)-tetraene, 1,3,5,14-tetraene, 1,3,5,15-tetraene, 1,3,5,16-tetraene, 1,3,6,8-tetraene, 1,3,6,8(14)-tetraene, 1,3,6,9-tetraene, 1,3,6,9(11)-tetraene, 1,3,6,12-tetraene, 1,3,6,13(17)-tetraene, 1,3,6,14-tetraene, 1,3,6,15-tetraene, 1,3,6,16-tetraene, 1,3,7,9-tetraene, 1,3,7,9(11)-tetraene, 1,3,7,11-tetraene, 1,3,7,12-tetraene, 1,3,7,13(17)-tetraene, 1,3,7,14-tetraene, 1,3,7,15-tetraene, 1,3,7,16-tetraene,1,3,8,9-tetraene, 1,3,8,9(11)-tetraene, 1,3,8,12-tetraene, 1,3,8,13(17)-tetraene, 1,3,8,14-tetraene, 1,3,8,15-tetraene, 1,3,8,16-tetraene, 1,3,8(14)9-tetraene, 1,3,8(14)9(11)-tetraene, 1,3,8(14)12-tetraene, 1,3,8(14)13(17)-tetraene, 1,3,8(14)15-tetraene, 1,3,8(14)16-tetraene, 1,3,9,11-tetraene, 1,3,9,12-tetraene, 1,3,9,13(17)-tetraene, 1,3,9,14-tetraene, 1,3,9,15-tetraene, 1,3,9,16-tetraene, 1,3,9(11),12-tetraene, 1,3,9(11),113(17)-tetraene, 1,3,9(11),14-tetraene, 1,3,9(11),15-tetraene, 1,3,9(11),16-tetraene, 1,3,12,14-tetraene, 1,3,12,15-tetraene, 1,3,12,16-tetraene, 1,3,13(17),14-tetraene, 1,3,13(17),15-tetraene, 1,3,13(17),16-tetraene, 1,3,14,16-tetraene, 1,4,6,8-tetraene, 1,4,6,8(14)-tetraene, 1,4,6,9-tetraene, 1,4,6,9(11)-tetraene, 1,4,6,11-tetraene, 1,4,6,12-tetraene, 1,4,6,13(17)-tetraene, 1,4,6,14-tetraene, 1,4,6,15-tetraene, 1,4,6,16-tetraene, 1,5,7,9-tetraene, 1,5,7,9(11)-tetraene, 1,5,7,11-tetraene, 1,5,7,12-tetraene, 1,5,7,13(17)-tetraene, 1,5,7,14-tetraene, 1,5,7,15-tetraene, 1,5,7,16-tetraene, 1,5,8,11-tetraene, 1,5,8,12-tetraene, 1,5,8,13(17)-tetraene, 1,5,8,14-tetraene, 1,5,8,15-tetraene, 1,5,8,16-tetraene, 1,5,8(14),9-tetraene, 1,5,8(14),9(11)-tetraene, 1,5,8(14),11-tetraene, 1,5,8(14),12-tetraene, 1,5,8(14),13(17)-tetraene, 1,5,8(14),15-tetraene, 1,5,8(14),16-tetraene, 1,5,9,11-tetraene, 1,5,9,12-tetraene, 1,5,9,13(17)-tetraene, 1,5,9,14-tetraene, 1,5,9,15-tetraene, 1,5,9,16-tetraene, 1,5,9(11),12-tetraene, 1,5,9(11),13(17)-tetraene, 1,5,9(11),14-tetraene, 1,5,9(11),15-tetraene, 1,5,9(11),16-tetraene, 1,5,11,13(17)-tetraene, 1,5,11,14-tetraene, 1,5,11,15-tetraene, 1,5,11,16-tetraene, 1,5,12,14-tetraene, 1,5,12,15-tetraene, 1,5,12,16-tetraene, 1,5,13(17),14-tetraene, 1,5,13(17),15-tetraene, 1,5,14,16-tetraene, 1,4,7,15-tetraene, 1,5,7,15-tetraene, 1,3,7,16-tetraene, 1,4,6,8-tetraene, 1,4,6,9-tetraene, 1,4,6,9(11)-tetraene, 1,4,6,11-tetraene, 1,4,6,12-tetraene, 1,4,6,13(17)-tetraene, 1,4,6,14-tetraene, 1,4,6,15-tetraene, 1,4,6,16-tetraene, 1,4,7,9-tetraene, 1,4,7,9(11)-tetraene, 1,4,7,11-tetraene, 1,4,7,12-tetraene, 1,4,7,13(17)-tetraene, 1,4,7,14-tetraene, 1,4,7,15-tetraene, 1,4,7,16-tetraene, 1,6,8,11-tetraene, 1,6,8,12-tetraene, 1,6,8,13(17)-tetraene, 1,6,8,14-tetraene, 1,6,8,15-tetraene, 1,6,8,16-tetraene, 1,6,8(14),9-tetraene, 1,6,8(14),9(11)-tetraene, 1,6,8(14),11-tetraene, 1,6,8(14),12-tetraene, 1,6,8(14),13(17)-tetraene, 1,6,8(14),15-tetraene, 1,6,8(14),16-tetraene, 1,6,9,11-tetraene, 1,6,9,12-tetraene, 1,6,9,13(17)-tetraene, 1,6,9,14-tetraene, 1,6,9,15-tetraene, 1,6,9,16-tetraene, 1,6,9(11),12-tetraene, 1,6,9(11),13(17)-tetraene, 1,6,9(11),14-tetraene, 1,6,9(11),15-tetraene, 1,6,9(11),16-tetraene, 1,6,11,13(17)-tetraene, 1,6,11,14-tetraene, 1,6,11,15-tetraene, 1,6,12,14-tetraene, 1,6,12,15-tetraene, 1,6,12,16-tetraene, 1,6,13(17),14-tetraene, 1,6,13(17),15-tetraene, 1,6,14,16-tetraene, 1,7,9,11-tetraene, 1,7,9,12-tetraene, 1,7,9,13(17)-tetraene, 1,7,9,14-tetraene, 1,7,9,15-tetraene, 1,7,9,16-tetraene, 1,8,11,13(17)-tetraene, 1,8,11,14-tetraene, 1,8,11,15-tetraene, 1,8,11,16-tetraene, 1,8(14),9,11-tetraene, 1,8(14),9,12-tetraene, 1,8(14),9,13(17)-tetraene, 1,8(14),9,15-tetraene, 1,8(14),9,16-tetraene, 1,9,11,13(17)-tetraene, 1,9,11,14-tetraene, 1,9,11,15-tetraene, 1,9,11,16-tetraene, 1,9(11),12,14-tetraene, 1,9(11),12,15-tetraene, 1,9(11),12,16-tetraene, 1,11,13(17),14-tetraene, 1,11,13(17),15-tetraene, 1,11,13(17),16-tetraene, 1,12,14,16-tetraene, 1,8,11,13(17)-tetraene, 1,8,11,14-tetraene, 1,8,11,15-tetraene, 1,9,11,13(17)-tetraene, 1,9,11,14-tetraene, 1,9,11,15-tetraene, 1,9,11,16-tetraene, 1,9(11),12,14-tetraene, 1,9(11),12,15-tetraene, 1,9(11),12,16-tetraene, 1,11,13(17),14-tetraene, 1,11,13(17),15-tetraene, 1,11,13(17),16-tetraene, 1,1-2,14,16-tetraene, 2,4,6,8-tetraene, 2,4,6,8(14)-tetraene, 2,4,6,9-tetraene, 2,4,6,9(11)-tetraene, 2,4,6,11-tetraene, 2,4,6,12-tetraene, 2,4,6,13(17)-tetraene, 2,4,6,14-tetraene, 2,4,6,15-tetraene, 2,4,6,16-tetraene, 2,5,7,9-tetraene, 2,5,7,9(11)-tetraene, 2,5,7,11-tetraene, 2,5,7,12-tetraene, 2,5,7,13(17)-tetraene, 2,5,7,14-tetraene, 2,5,7,15-tetraene, 2,5,7,16-tetraene, 2,5,8,11-tetraene, 2,5,8,12-tetraene, 2,5,8,13(17)-tetraene, 2,5,8,14-tetraene, 2,5,8,15-tetraene, 2,5,8,16-tetraene, 2,5,8(14),9-tetraene, 2,5,8(14),9(11)-tetraene, 2,5,8(14),11-tetraene, 2,5,8(14),16-tetraene, 2,5,8(14),13(17)-tetraene, 2,5,8(14),15-tetraene, 2,5,8(14),16-tetraene, 2,5,9,11-tetraene, 2,5,9,12-tetraene, 2,5,9,13(17)-tetraene, 2,5,9,14-tetraene, 2,5,9,15-tetraene, 2,5,9,16-tetraene, 2,5,9(11),12-tetraene, 2,5,9(11),13(17)-tetraene, 2,5,9(11),14-tetraene, 2,5,9(11),15-tetraene, 2,5,9(11),16-tetraene, 2,5,11,13(17)-tetraene, 2,5,11,14-tetraene, 2,5,11,15-tetraene, 2,5,11,16-tetraene, 2,5,12,14-tetraene, 2,5,12,15-tetraene, 2,5,12,16-tetraene, 2,5,13(17),14-tetraene, 2,5,13(17),15-tetraene, 2,5,14,16-tetraene, 2,4,7,15-tetraene, 2,5,7,15-tetraene, 2,4,6,8-tetraene, 2,4,6,9-tetraene, 2,4,6,9(11)-tetraene, 2,4,6,11-tetraene, 2,4,6,12-tetraene, 2,4,6,13(17)-tetraene, 2,4,6,14-tetraene, 2,4,6,15-tetraene, 2,4,6,16-tetraene, 2,4,7,9-tetraene, 2,4,7,9(11)-tetraene, 2,4,7,11-tetraene, 2,4,7,12-tetraene, 2,4,7,13(17)-tetraene, 2,4,7,14-tetraene, 2,4,7,15-tetraene, 2,4,7,16-tetraene, 2,6,8,11-tetraene, 2,6,8,12-tetraene, 2,6,8,13(17)-tetraene, 2,6,8,14-tetraene, 2,6,8,15-tetraene, 2,6,8,16-tetraene, 2,6,8(14),9-tetraene, 2,6,8(14),9(11)-tetraene, 2,6,8(14),11-tetraene, 2,6,8(14),12-tetraene, 2,6,8(14),13(17)-tetraene, 2,6,8(14),15-tetraene, 2,6,8(14),16-tetraene, 2,6,9,11-tetraene, 2,6,9,12-tetraene, 2,6,9,13(17)-tetraene, 2,6,9,14-tetraene, 2,6,9,15-tetraene, 2,6,9,16-tetraene, 2,6,9(11),12-tetraene, 2,6,9(11),13(17)-tetraene, 2,6,9(11),14-tetraene, 2,6,9(11),15-tetraene, 2,6,9(11),16-tetraene, 2,6,11,13(17)-tetraene, 2,6,11,14-tetraene, 2,6,11,15-tetraene, 2,6,12,14-tetraene, 2,6,12,15-tetrane, 2,6,12,16-tetrane, 2,6,13(17),14-tetraene, 2,6,13(17),15-tetraene, 2,6,14,16-tetraene, 2,7,9,11-tetraene, 2,7,9,12-tetraene, 2,7,9,13(17)-tetraene, 2,7,9,14-tetraene, 2,7,9,15-tetraene, 2,7,9,16-tetraene, 2,8,11,13(17)-tetraene, 2,8,11,14-tetraene, 2,8,11,15-tetraene, 2,8,11,16-tetraene, 2,8(14),9,11-tetraene, 2,8(14),9,12-tetraene, 2,8(14),9,13(17)-tetraene, 2,8(14),9,15-tetraene, 2,8(14),9,16-tetraene, 2,9,11,13(17)-tetraene, 2,9,11,14-tetraene, 2,9,11,15-tetraene, 2,9,11,16-tetraene, 2,9(11),12,14-tetraene, 2,9(11),12,15-tetraene, 2,9(11),12,16-tetraene, 2,11,13(17),14-tetraene, 2,11,13(17),15-tetraene, 2,11,13(17),16-tetraene, 2,12,14,16-tetraene, 2,8,11,13(17)-tetraene, 2,8,11,14-tetraene, 2,8,11,15-tetraene, 2,9,11,13(17)-tetraene, 2,9,11,14-tetraene, 2,9,11,15-tetraene, 2,9,11,16-tetraene, 2,9(11),12,14-tetraene, 2,9(11),12,15-tetraene, 2,9(11),12,16-tetraene, 2,11,13(17),14-tetraene, 2,11,13(17),15-tetraene, 2,11,13(17),16-tetraene, 2,12,14,16-tetraene, 3,5,7,9-tetraene, 3,5,7,9(11)-tetraene, 3,5,7,11-tetraene, 3,5,7,12-tetraene, 3,5,7,13(17)-tetraene, 3,5,7,14-tetraene, 3,5,7,15-tetraene, 3,5,7,16-tetraene, 3,5,8,11-tetraene, 3,5,8,12-tetraene, 3,5,8,13(17)-tetraene, 3,5,8,14-tetraene, 3,5,8,15-tetraene, 3,5,8,16-tetraene, 3,5,8(14),9-tetraene, 3,5,8(14),9(11)-tetraene, 3,5,8(14),11-tetraene, 3,5,8(14),12-tetraene, 3,5,8(14),13(17)-tetraene, 3,5,8(14),15-tetraene, 3,5,8(14),16-tetraene, 3,5,9,11-tetraene, 3,5,9,12-tetraene, 3,5,9,13(17)-tetraene, 3,5,9,14-tetraene, 3,5,9,15-tetraene, 3,5,9,16-tetraene, 3,5,9(11),12-tetraene, 3,5,9(11),13(17)-tetraene, 3,5,9(11),14-tetraene, 3,5,9(11),15-tetraene, 3,5,9(11),16-tetraene, 3,5,11,13(17)-tetraene, 3,5,11,14-tetraene, 3,5,11,15-tetraene, 3,5,11,16-tetraene, 3,5,12,14-tetraene, 3,5,12,15-tetraene, 3,5,12,16-tetraene, 3,5,13(17),14-tetraene, 3,5,13(17),15-tetraene, 3,5,14,16-tetraene, 3,4,7,15-tetraene, 3,5,7,15-tetraene, 3,5,7,16-tetraene, 3,4,6,8-tetraene, 3,4,6,9-tetraene, 3,4,6,9(11)-tetraene, 3,4,6,11-tetraene, 3,4,6,12-tetraene, 3,4,6,13(17)-tetraene, 3,4,6,14-tetraene, 3,4,6,15-tetraene, 3,4,6,16-tetraene, 3,4,7,9-tetraene, 3,4,7,9(11)-tetraene, 3,4,7,11-tetraene, 3,4,7,12-tetraene, 3,4,7,13(17)-tetraene, 3,4,7,14-tetraene, 3,4,7,15-tetraene, 3,4,7,16-tetraene, 3,6,8,11-tetraene, 3,6,8,12-tetraene, 3,6,8,13(17)-tetraene, 3,6,8,14-tetraene, 3,6,8,15-tetraene, 3,6,8,16-tetraene, 3,6,8(14),9-tetraene, 3,6,8(14),9(11)-tetraene, 3,6,8(14),11-tetraene, 3,6,8-tetraene, 3,6,8(14),13(17)-tetraene, 3,6,8(14),15-tetraene, 3,6,8(14),16-tetraene, 3,6,9,11-tetraene, 3,6,9,12-tetraene, 3,6,9,13(17)-tetraene, 3,6,9,14-tetraene, 3,6,9,15-tetraene, 3,6,9,16-tetraene, 3,6,9(11),12-tetraene, 3,6,9(11),13(17)-tetraene, 3,6,9(11),14-tetraene, 3,6,9(11),15-tetraene, 3,6,9(11),16-tetraene, 3,6,11,13(17)-tetraene, 3,6,11,14-tetraene, 3,6,11,15-tetraene, 3,6,12,14-tetrane, 3,6,12,15-tetrane, 3,6,12,16-tetrane, 3,6,13(17),14-tetraene, 3,6,13(17),15-tetraene, 3,6,14,16-tetraene, 3,7,9,11-tetraene, 3,7,9,12-tetraene, 3,7,9,13(17)-tetraene, 3,7,9,14-tetraene, 3,7,9,15-tetraene, 3,7,9,16-tetraene, 3,8,11,13(17)-tetraene, 3,8,11,14-tetraene, 3,8,11,15-tetraene, 3,8,11,16-tetraene, 3,8(14),9,11-tetraene, 3,8(14),9,12-tetraene, 3,8(14),9,13(17)-tetraene, 3,8(14),9,15-tetraene, 3,8(14),9,16-tetraene, 3,9,11,13(17)-tetraene, 3,9,11,14-tetraene, 3,9,11,15-tetraene, 3,9,11,16-tetraene, 3,9(11),12,14-tetraene, 3,9(11),12,15-tetraene, 3,9(11),12,16-tetraene, 3,11,13(17),14-tetraene, 3,11,13(17),15-tetraene, 3,11,13(17),16-tetraene, 3,12,14,16-tetraene, 3,8,11,13(17)-tetraene, 3,8,11,14-tetraene, 3,8,11,15-tetraene, 3,9,11,13(17)-tetraene, 3,9,11,14-tetraene, 3,9,11,15-tetraene, 3,9,11,16-tetraene, 3,9(11),12,14-tetraene, 3,9(11),12,15-tetraene, 3,9(11),12,16-tetraene, 3,11,13(17),14-tetraene, 3,11,13(17),15-tetraene, 3,11,13(17),16-tetraene, 3,12,14,16-tetraene, 3,5(10),7,9(11)-tetraene, 3,5(10),7,11-tetraene, 3,5(10),7,12-tetraene, 3,5(10),7,13(17)-tetraene, 3,5(10),7,14-tetraene, 3,5(10),7,15-tetraene, 3,5(10),7,16-tetraene, 3,5(10),8,11-tetraene, 3,5(10),8,12-tetraene, 3,5(10),8,13(17)-tetraene, 3,5(10),8,14-tetraene, 3,5(10),8,15-tetraene, 3,5(10),8,16-tetraene, 3,5(10),8(14),9-tetraene, 3,5(10),8(14),9(11)-tetraene, 3,5(10),8(14),11-tetraene, 3,5(10),8(14),12-tetraene, 3,5(10),8(14),13(17)-tetraene, 3,5(10),8(14),15-tetraene, 3,5(10),8(14),16-tetraene, 3,5(10),9,11-tetraene, 3,5(10),9,12-tetraene, 3,5(10),9,13(17)-tetraene, 3,5(10),9,14-tetraene, 3,5(10),9,15-tetraene, 3,5(10),9,16-tetraene, 3,5(10),9(11),12-tetraene, 3,5(10),9(11),13(17)-tetraene, 3,5(10),9(11),14-tetraene, 3,5(10),9(11),15-tetraene, 3,5(10),9(11),16-tetraene, 3,5(10),11,13(17)-tetraene, 3,5(10),11,14-tetraene, 3,5(10),11,15-tetraene, 3,5(10),11,16-tetraene, 3,5(10),12,14-tetraene, 3,5(10),12,15-tetraene, 3,5(10),12,16-tetraene, 3,5(10),13(17),14-tetraene, 3,5(10),13(17),15-tetraene, 3,5(10),14,16-tetraene, 4,6,8,11-tetraene, 4,6,8,12-tetraene, 4,6,8,13(17)-tetraene, 4,6,8,14-tetraene, 4,6,8,15-tetraene, 4,6,8,16-tetraene, 4,6,8(14),9-tetraene, 4,6,8(14),9(11)-tetraene, 4,6,8(14),11-tetraene, 4,6,8(14),12-tetraene, 4,6,8(14),13(17)-tetraene, 4,6,8(14),15-tetraene, 4,6,8(14),16-tetraene, 4,6,9,11-tetraene, 4,6,9,12-tetraene, 4,6,9,13(17)-tetraene, 4,6,9,14-tetraene, 4,6,9,15-tetraene, 4,6,9,16-tetraene, 4,6,9(11),12-tetraene, 4,6,9(11),13(17)-tetraene, 4,6,9(11),14-tetraene, 4,6,9(11),15-tetraene, 4,6,9(11),16-tetraene, 4,6,11,13(17)-tetraene, 4,6,11,14-tetraene, 4,6,11,15-tetraene, 4,6,12,14-tetrane, 4,6,12,15-tetrane, 4,6,12,16-tetrane, 4,6,13(17),14-tetraene, 4,6,13(17),15-tetraene, 4,6,14,16-tetraene, 4,7,9,11-tetraene, 4,7,9,12-tetraene, 4,7,9,13(17)-tetraene, 4,7,9,14-tetraene, 4,7,9,15-tetraene, 4,7,9,16-tetraene, 4,8,11,13(17)-tetraene, 4,8,11,14-tetraene, 4,8,11,15-tetraene, 4,8,11,16-tetraene, 4,8(14),9,11-tetraene, 4,8(14),9,12-tetraene, 4,8(14),9,13(17)-tetraene, 4,8(14),9,15-tetraene, 4,8(14),9,16-tetraene, 4,9,11,13(17)-tetraene, 4,9,11,14-tetraene, 4,9,11,15-tetraene, 4,9,11,16-tetraene, 4,9(11),12,14-tetraene, 4,9(11),12,15-tetraene, 4,9(11),12,16-tetraene, 4,11,13(17),14-tetraene, 4,11,13(17),15-tetraene, 4,11,13(17),16-tetraene, 4,12,14,16-tetraene, 4,8,11,13(17)-tetraene, 4,8,11,14-tetraene, 4,8,11,15-tetraene, 4,9,11,13(17)-tetraene, 4,9,11,14-tetraene, 4,9,11,15-tetraene, 4,9,11,16-tetraene, 4,9(11),12,14-tetraene, 4,9(11),12,15-tetraene, 4,9(11),12,16-tetraene, 4,11,13(17),14-tetraene, 4,11,13(17),15-tetraene, 4,11,13(17),16-tetraene, 4,12,14,16-tetraene, 5,7,9,11-tetraene, 5,7,9,12-tetraene, 5,7,9,13(17)-tetraene, 5,7,9,14-tetraene, 5,7,9,15-tetraene, 5,7,9,16-tetraene, 5,8,11,13(17)-tetraene, 5,8,11,14-tetraene, 5,8,11,15-tetraene, 5,8,11,16-tetraene, 5,8(14),9,11-tetraene, 5,8(14),9,12-tetraene, 5,8(14),9,13(17)-tetraene, 5,8(14),9,15-tetraene, 5,8(14),9,16-tetraene, 5,9,11,13(17)-tetraene, 5,9,11,14-tetraene, 5,9,11,15-tetraene, 5,9,11,16-tetraene, 5,9(11),12,14-tetraene, 5,9(11),12,15-tetraene, 5,9(11),12,16-tetraene, 5,11,13(17),14-tetraene, 5,11,13(17),15-tetraene, 5,11,13(17),16-tetraene, 5,12,14,16-tetraene, 5,8,11,13(17)-tetraene, 5,8,11,14-tetraene, 5,8,11,15-tetraene, 5,9,11,13(17)-tetraene, 5,9,11,14-tetraene, 5,9,11,15-tetraene, 5,9,11,16-tetraene, 5,9(11),12,14-tetraene, 5,9(11),12,15-tetraene, 5,9(11),12,16-tetraene, 5,11,13(17),14-tetraene, 5,11,13(17),15-tetraene, 5,11,13(17),16-tetraene, 5,12,14,1.6-tetraene, 5(10),8,11,13(17)-tetraene, 5(10),8,11,14-tetraene, 5(10),8,11,15-tetraene, 5(10),8,11,16-tetraene, 5(10),8(14),9,11-tetraene, 5(10),8(14),9,12-tetraene, 5(10),8(14),9,13(17)-tetraene, 5(10),8(14),9,15-tetraene, 5(10),8(14),9,16-tetraene, 5(10),9,11,13(17)-tetraene, 5(10),9,11,14-tetraene, 5(10),9,11,15-tetraene, 5(10),9,11,16-tetraene, 5(10),9(11),12,14-tetraene, 5(10),9(11),12,15-tetraene, 5(10),9(11),12,16-tetraene, 5(10),11,13(17),14-tetraene, 5(10),11,13(17),15-tetraene, 5(10),11,13(17),16-tetraene, 5(10),12,14,16-tetraene, 5(10),8,11,13(17)-tetraene, 5(10),8,11,14-tetraene, 5(10),8,11,15-tetraene, 5(10),9,11,13(17)-tetraene, 5(10),9,11,14-tetraene, 5(10),9,11,15-tetraene, 5(10),9,11,16-tetraene, 5(10),9(11),12,14-tetraene, 5(10),9(11),12,15-tetraene, 5(10),9(11),12,16-tetraene, 5(10),11,13(17),14-tetraene, 5(10),11,13(17),15-tetraene, 5(10),11,13(17),16-tetraene, 5(10),12,14,16-tetraene, 6,8,11,13(17)-tetraene, 6,8,11,14-tetraene, 6,8,11,15-tetraene, 6,8,11,16-tetraene, 6,9,11,13(17)-tetraene, 6,9,11,14-tetraene, 6,9,11,15-tetraene, 6,9,11,16-tetraene, 6,9(11),12,14-tetraene, 6,9(11),12,15-tetraene, 6,9(11),12,16-tetraene, 6,11,13(17),14-tetraene, 6,11,13(17),15-tetraene, 6,12,14,16-tetraene, 7,9,11,13(17)-tetraene, 7,9,11,14-tetraene, 7,9,11,15-tetraene, 7,9,11,16-tetraene, 7,9(11),12,14-tetraene, 7,9(11),12,15-tetraene, 7,9(11),12,16-tetraene, 8,11,13(17),14-tetraene, 8,11,13(17),15-tetraene, 8(14),9,11,13(17)-tetraene, 8(14),9,11,15-tetraene, 8(14),9,11,16-tetraene, 9,11,13(17),14-tetraene, 9,11,13(17),15-tetraene, 9(11),12,14,16-tetraene, 11,13(17),14,16-tetraene, 1,3,5(10),6,8-pentaene, 1,3,5(10),6,9(11)-pentaene, 1,3,5(10),6,11-pentaene, 1,3,5(10),6,12-pentaene, 1,3,5(10),6,13(17)-pentaene, 1,3,5(10),6,14-pentaene, 1,3,5(10),6,15-pentaene, 1,3,5(10),6,16-pentaene, 1,3,5(10),7,9(11)-pentaene, 1,3,5(10),7,11-pentaene, 1,3,5(10),7,12-pentaene, 1,3,5(10),7,13(17)-pentaene, 1,3,5(10),7,14-pentaene, 1,3,5(10),7,15-pentaene, 1,3,5(10),7,16-pentaene, 1,3,5(10),8,11-pentaene, 1,3,5(10),8,12-pentaene, 1,3,5(10),8,13(17)-pentaene, 1,3,5(10),8,14-pentaene, 1,3,5(10),8,15-pentaene, 1,3,5(10),8,16-pentaene, 1,3,5(10),8(14),9(11)-pentaene, 1,3,5(10),8(14),11-pentaene, 1,3,5(10),8(14),12-pentaene, 1,3,5(10),8(14),13(17)-pentaene, 1,3,5(10),8(14),15-pentaene, 1,3,5(10),8(14),16-pentaene, 1,3,5(10),9(11),12-pentaene, 1,3,5(10),9(11),13(17)-pentaene, 1,3,5(10),9(11),14-pentaene, 1,3,5(10),9(11),15-pentaene, 1,3,5(10),9(11),16-pentaene, 1,3,5(10),11,13(17)-pentaene, 1,3,5(10),11,14-pentaene, 1,3,5(10),11,15-pentaene, 1,3,5(10),11,16-pentaene, 1,3,5(10),12,14-pentaene, 1,3,5(10),12,15-pentaene, 1,3,5(10),12,16-pentaene, 1,3,5(10),13(17),14-pentaene, 1,3,5(10),13(17),15-pentaene or a 1,3,5(10),14,16-pentaene androstene.

Methods to make related compounds been described, see, e.g., U.S. Pat. Nos. 2,833,793, 2,911,418, 3,148,198, 3,471,480, 3,976,691, 4,000,125, 4,083,969, 4,268,441, 4,427,649, 4,542,129, 4,666,898, 4,956,355, 5,001,119, 5,043,165, 5,077,284, 5,028,631, 5,110,810, 5,157,031, 5,162,198, 5,175,154, 5,277,907, 5,292,730, 5,296,481, 5,372,996, 5,387,583, 5,407,684, 5,424,463, 5,461,042, 5,478,566, 5,506,223, 5,518,725, 5,527,788, 5,527,789, 5,532,230, 5,559,107, 5,562,910, 5,583,126, 5,585,371, 5,587,369, 5,591,736, 5,593,981, 5,629,295, 5,610,150, 5,635,496, 5,641,766, 5,641,768, 5,656,621, 5,660,835, 5,686,438, 5,696,106, 5,700,793, 5,707,983, 5,709,878, 5,710,143, 5,714,481, 5,728,688, 5,736,537, 5,744,462, 5,753,237, 5,756,482, 5,776,921, 5,776,923, 5,780,460, 5,795,880, 5,798,347, 5,798,348, 5,804,576, 5,807,848, 5,807,849, 5,811,418, 5,824,313, 5,824,668, 5,824,671, 5,827,841, 5,837,269, 5,837,700, 5,843,932, 5,846,963, 5,859,000, 5,872,114, 5,872,147, 5,162,198, 5,206,008, 5,292,730, 5,407,684, 5,461,042, 5,461,768, 5,478,566, 5,585,371, 5,635,496, 5,641,766, 5,837,269, 5,885,977, 5,846,963, 5,919,465, 5,869,090, 5,863,910, 5,856,340, 5,804,576, 5,714,481, 6,150,336, 4,978,532, 4,898,694, 4,542,129, 3,711,606, 3,710,795, 3,189,597, 3,137,710, 2,531,441, 4,908,358, 4,902,681, 5,532,230, 5,686,438, 5,753,640, 5,811,418, 5,859,000, 5,763,433, 6,372,732, 5,925,630, 5,939,545 and 5,962,443.

Modulator compounds. Compounds suitable for use in the invention include the compounds described in U.S. Pat. Nos. 6,784,167, 6,541,463, 6,423,698, 6,124,115, 5,817,649, 5,595,985, 5,550,107, 5,439,943, 5,399,790, and 5,118,621. In general, dosages of such compounds will range from about 0.05 mg/kg/day to about 100 mg/kg/day on days when the compounds are used, e.g., about 0.1 or 1 mg/kg/day to about 20 or 30 mg/kg/day. Typically the modulator compounds will be administered for up to about 21 days beginning within about 1, 2 or 3 days before the exposure to a biological insult such as a radiation exposure, or for up to about 21 days beginning within 1 or two hours up to about 2 or 3 days after the exposure to the biological insult.

Determination of the status profile. Some aspects of the invention and related subject matter center on (i) methods to determine the status profile for a subject or groups of subjects that have been exposed to a biological insult that is potentially life-threatening and (ii) identification of biological parameters, typically biological results or symptoms of the biological insult, that can be used to obtain a status profile. The status profile is a predictor of survival after exposure of a subject to a potentially lethal biological insult. A survival status profile, or Pₛᵤᵣᵥᵢᵥₐₗ, is the probability that the subject will survive the biological insult, absent treatment other than palliative treatments such as management of symptoms, pain, fever, suffering, nutrition, body or peripheral temperature, water or electrolyte management or other typical palliative treatments. A lethality status profile, or P_{lethality}, is the probability that the subject will not survive the biological insult, absent treatment other than palliative treatments such as management of symptoms, pain, fever, suffering, nutrition, body or peripheral temperature, water or electrolyte management or other typical palliative treatments. As the foregoing indicates, methods to obtain Pₛᵤᵣᵥᵢᵥₐₗ or P_{lethality} that are highly reliable are useful for many purposes, e.g., to assess or diagnose a subject's clinical condition or prognosis or to tailor palliative or other therapies to fit the subject's clinical condition. This information is particularly useful where the status profile is obtained soon, e.g., within about 12-48 hours, after a biological insult that can cause death at a much later time, e.g., at about 1, 2 or 3 weeks later.

When the subject species is a human, the exposed patients are intended to be treated with at least the minimal acceptable treatment consistent with the subject's clinical condition and/or the biological insult and/or the subject's local clinical standards of care and/or any standard of care that is practical under the circumstances. In cases where medical care is limited or at least temporarily unavailable, measurements, e.g., non-invasive measurements of temperature, to obtain status profile information can be obtained. Such information can be used to assess or triage the patient.

In general Pₛᵤᵣᵥᵢᵥₐₗ or P_{lethality} values that can be stated with high precision are of the greatest interest or, for human clinical practice, utility. As used herein, any Pₛᵤᵣᵥᵢᵥₐₗ means that the value predicts survival of the exposed subject with at least about a 80%, 85%, 90%, 91%, 92%, 93%, 94% degree of confidence, or preferably at least about a 95% degree of confidence, which is considered statistically acceptable. Similarly, a P_{lethality} means that the value predicts non-survival of the exposed subject with at least about 80%, 85%, 90%, 91%, 92%, 93%, 94% degree of confidence, or preferably at least about a 95% degree of confidence. Typically, Pₛᵤᵣᵥᵢᵥₐₗ values of at least about 0.8, at least about 0.85, at least about 0.9, at least about 0.92, at least about 0.93, at least about 0.94, at least about 0.95, at least about 0.96, at least about 0.97, at least about 0.98, at least about 0.99, at least about 0.995, at least about 0.999 or better are generally useful in the [symbol] P_{lethality} values of [symbol] 0.2, about 0.15, about 0.1, about 0.08, about 0.07, about 0.06, about 0.05, about 0.04, about [symbol] 0.02, about 0.01, about 0.005, about 0.001 or better are generally useful in the invention.

In some of these embodiments, the invention provides methods to determine a subject's status profile, where the methods comprise, (1) exposing the subject to a sufficient amount of a biological insult (or exposing a group of subjects, where the group has been exposed to the same, essentially the same or a similar, but comparable biological insult) to potentially (e.g., the probability is at least 10%, about 30%, about 50% or more to at least about 60% or about 70%, about 80% or more) cause or elicit one, two or more biological responses that are potentially life-threatening to obtain an exposed subject (or group of subjects); (2) measuring on 1, 2, 3, 4 or more occasions in or from the exposed subject (or group of subjects) 1, 2, 3, 4 or more parameters selected from temperature, red blood cell counts, hematocrit, red blood cell precursors optionally selected from CFU-GEMM, BFU-E, CFU-E, proerythroblasts, pronormoblasts, basophilic normoblasts, polychromatic normoblasts, orthochromatic normoblasts and reticulocytes, platelets, platelet precursors optionally selected from megakaryocytes, megakaryocyte progenitor cells, megakaryocyte precursor cells, promegakaryoblasts, immature megakaryocyte colony forming units, mature megakaryocyte colony forming units and megakaryocyte lineage markers optionally selected from GP-IIb, GP-IX, PF4 and GP-Ibα, macrophages, monocytes or monocyte precursors optionally selected from CD34⁻CD90⁺CD123⁺CD117⁺CD135⁺ stem cells, CD34⁺CD33⁻CD38⁻CD45RO⁺CD45RA⁻ progenitor cells, CFU-GEMM (e.g., CD34⁺CD33⁺CD38⁻), CFU-GM (e.g., CD64⁺), CFU-M (e.g., CD34⁺CD33⁺CDC13⁺), monoblasts (e.g., CD33⁺CD38⁺CD14⁺), promonocytes (e.g., CD64⁺CD11c⁺CD14⁺), C reactive protein, nitric oxide or inducible or constitutive nitric oxide synthetase levels, fibrinogen, sepsis, respiration rate, pulse rate, blood or arterial pH, blood pressure, pH or composition of sweat, pH or composition of saliva, respired breath composition, pheromone composition, urine or feces pH or composition, blood SaO₂ or oxygen saturation of arterial oxyhemoglobin (e.g., as measured by a pulse oximeter), a circadian, diurnal or nocturnal rhythm parameter, optionally selected from one, two or more of rapid eye movement sleep, sleeping brain theta waves, leptin, glucose, insulin, melatonin, heart rate, temperature, locomotor activity, autonomic nervous function, hormone, glucocorticoid such as cortisol, blood enzyme levels, B-cells, T-cells, natural killer cells, dendritic cells, neutrophils, eosinophils, basophils, CFU-Eos, CFU-Baso or a progenitor or precursor of any of these such as a neutrophil or other precursor optionally selected from CD34⁻CD90⁺CD123⁺CD117⁺CD135⁺ stem cells, CD34⁺CD33⁻CD38⁻CD45RO⁺CD45RA⁻ progenitor cells, CFU-GEMM (e.g., CD34⁺CD33⁺CD38⁻), CFU-GM (e.g., CD64⁺), CFU-G (e.g., CD45RA⁺MPO³⁰ ), myeloblasts (e.g., CD33⁺CD38⁺), complement protein C3a, sepsis, e.g., as determined by detection of bacteria in blood, liver, lung or other tissue on 1, 2, 3 or more occasions, septic shock, myelocytes, neurological damage (e.g., motor function impairment, cognitive impairment or autonomic function impairment), wherein the measurements are obtained at times before, during or overlapping with, and/or after the biological insult to obtain a status profile for the exposed subject or the treated exposed subject (or the exposed group of subjects, and/or the exposed treated group of subjects); (3) optionally administering one or more palliative or ameliorative therapies to treat one or more side effects of the biological insult to obtain an exposed treated subject(s); (4) measuring the survival rate of the exposed subject(s) and/or the exposed treated subject(s); and (5) identifying one or more status profiles that corresponds to a defined probability of surviving the biological insult (Pₛᵤᵣᵥᵢᵥₐₗ) or of not surviving the biological insult (P_{lethality}). Types of mature blood cells, their progenitors and methods to measure or identify them have been described, e.g., Hematology—Basic Principles and Practice, 3^{rd} edition, R. Hoffman, E. J. Benz Jr. et al., editors, Churchill Livingstone, New York, 2000, see, e.g., chapter 12 at pages 126-138 and chapter 13 at pages 139-154, chapter 15 at pages 202-219, chapter 16 at pages 220-222 and chapter 17 at pages 245-260. These methods and descriptions can be used in the invention methods. Statistical analysis methods, e.g., Baye's rule, that can be used in or adapted to the invention methods have been described. B. Rosner, Fundamentals of Biostatistics, 2^{nd} ed. 1986, chapters 1-12, e.g., pages 42-136, PWS Publishers, Duxbury Press, Boston, Mass., D. G. Altman, Practical Statistics for Medical Research, 1^{st} ed., 1990, Chapman & Hall/CRC Press, e.g., pages 1-616, ISBN 0412276305.

In these embodiments, the biological insult typically comprises exposure of one or more subjects to one or more of radiation, toxin, trauma and/or chemotherapy. Biological responses to a biological insult that is potentially life-threatening can be associated with a variety of conditions, e.g., a toxicity or tissue damage from an infectious agent, side-effects of trauma such as blood loss, and/or impairment, failure or death of one or more organs or tissues, e.g., kidney, liver, heart, intestine, stomach or skin or bone marrow failure or impairment after exposure to radiation or a toxic chemotherapy. To obtain measurements for assembling a status profile, cells or tissue can be obtained from marrow, spleen, thymus, lymph node, lymph fluid, liver or lung blood, serum or tissue from the exposed subject(s) and/or the exposed treated subject(s). Types of mature blood cell, their progenitors and methods to measure or identify them have been described, e.g., Hematology—Basic Principles and Practice, 3^{rd} edition, R. Hoffman, E. J. Benz Jr. et al., editors, Churchill Livingstone, New York, 2000, see, e.g., chapter 12 at pages 126-138 and chapter 13 at pages 139-154, chapter 15 at pages 202-219, chapter 16 at pages 220-222 and chapter 17 at pages 245-260.

For small subjects such as mice or rats, measurement of some parameters, e.g., measuring a particular cell type in bone marrow tissue, on more than one occasion may not be easily accomplished. In these situations, obtaining more than one measurement of a parameter will thus typically be accomplished using measurements from one or more exposed subjects or exposed treated subjects once and other one or more exposed subjects or exposed treated subjects (s) at one or two other occasions to get the needed time points. Exposure of subjects to a biological insult such a radiation exposure can be controlled to within about 2%, about 3%, about 4%, about 5%, about 6%, about 7%, about 8%, about 9%, or about 10% of a desired dose or lethality level. This can be accomplished by calibratiion using, e.g., an acrylic phantom(s) placed in the same experimental set up that is used for animal irradiation. Such calibration will take into account the body weight, body size and shape of subjects such as rodents or non-human primates, e.g., chest circumference, weight and leg length and total body height.

For radiation of non-human primates, the radiation dose can be calibrated prior to conducting a study using two acrylic phantoms placed in the same experimental set up that would be used for animal irradiation. Exposure time for each animal can be calculated based on, e.g., the circumference of each animal at the junction of the thorax and the abdomen. The actual dose received may be determined using ONE DOSE® or by other known protocols, see e.g., M. G. Stabin, Cancer Biotherapy and Radiopharmaceuticals, 18(4):611-617 2003. Four dosimeters can be used for each animal. The dosimeters can be placed on the sagittal plane of the animal on the sternal, interscapular, lumbar and lower abdominal regions. Dosimetry measurements using phantom and One Dose dosimeters, the dose rate, duration of irradiation and the actual time of irradiation for each individual animal can thus be determined.

In these methods, the biological insult may comprise exposure of the subjects to, e.g., radiation or chemotherapy, optionally wherein the exposure is about an LD₂ or an LD₅ to about LD₉₀ or an LD₅₀₀. As used here LD₂ means an injury or insult that would on average lead to death of 2% of exposed subjects, while LD₅₀ means an injury or insult that would on average lead to death of 50% of exposed subjects. The biological insult, e.g., radiation dose, can be about an LD_{0.1}, about an LD_{0.5}, about an LD₁, about an LD₂, about an LD₅, about an LD₁₀, about an LD₂₀, about an LD₃₀, about an LD₄₀, about an LD₅₀, about an LD₆₀, about an LD₇₀, about an LD₈₀, about an LD₉₀, about an LD₁₀₀, or a dose that is about 1.1, 1.2, 1.3, 1.4, 1.5, 2, 2.5, 3, 3.5, 4, 4.5 or 5 fold higher than a LD₁₀₀ dose or a dose within any range between any two of these values, e.g., from about an LD₅, about an LD₂₀ or about an LD₄₀, to about an LD₅₅, about an LD₆₀ or about an LD₉₀. Exemplary radiation dose ranges or exposures include about an LD₅₀, about an LD₃₀ to about an LD₇₀ dose or exposure or about an LD₄₀ to about an LD₆₀ dose or exposure. For any of these biological insults that can elicit one, two or more biological responses that are potentially life-threatening, the time of survival will usually be determined at 30 days or at 60 days after the biological insult has occurred. An LD_{50/60} is a 50% survival rate at 60 days after the biological insult, while an LD_{50/30} is a 50% survival rate at 30 days after the biological insult. For humans, survival is often determined at 60 days after exposure and for other subjects survival is typically determined at about 20 days, 30 days or 60 days after exposure.

Where the biological insult is accidental or intentional exposure to radiation, the radiation may comprise one, two or more of γ-radiation, x-radiation, α-radiation, β-radiation, fast neutron radiation, slow neutron radiation, cosmic radiation, ultraviolet A radiation, ultraviolet B radiation, microwave radiation, ⁶⁰Co radiation, ¹³⁷Cs radiation, ⁸⁹Sr, ⁹⁰Sr, ¹³¹I, ³²P, ³⁵S, ²⁴Na, ³²K, ¹³¹Ce, ¹⁴⁴Ce, ²³⁵U and/or heavy particle radiation, e.g., silicon or boron particle radiation. Survival rates can be measured at 30 days and/or at 60 days after exposure to the radiation. Radiation exposure can arise from an external source, inhaled radioactive material, ingested radioactive material and/or implanted radioactive material, any of which may arise from an accidental exposure or from an intentional exposure, e.g., for a therapy.

The biological insult will typically occur over a relatively short period of time, e.g., over a period of from less than about a minute or about 5 minutes to about 1 hour or about 2 hours. In some cases, the biological insult, e.g., tissue damage from a trauma such as surgery, a serious wound or a skin or chemical burn, can occur over a longer time, e.g., over about 2 hours or about 3 hours to about 4 hours, about 12 hours or about 1, 2, 3 or more days. In these cases, the time of the biological insult can be considered to be at about the time when any significant injury has occurred or when acute aspects or symptoms of the injury have had time to become apparent or to cause significant tissue or organ impairment. Specific types of biological insult that are applicable to these methods are as described elsewhere herein, including one or more of radiation exposure, toxin or poison exposure or ingestion, chemotherapy including myelosuppressive therapy and glucocorticoid therapy, infection, cancer, ischemia, hemorrhage, stroke or other trauma conditions. Typically the biological insult is of a sufficient magnitude to elicit a potentially life-threatening biological response.

In any of these methods, a status profile corresponding to (i) a defined probability of individual subject surviving the biological insult, Pₛᵤᵣᵥᵢᵥₐₗ, or (ii) a defined probability of an individual subject not surviving the biological insult, P_{lethality}, is obtained in step (5). The status profile will typically be obtained for a group of exposed subjects. However, a single individual can be used to obtain a status profile, usually when a suitable comparator status profile is available. For example, when a status profile is available for a species such as rhesus macaque, cynomolgus macaque or a chimpanzee, the status profile can be applied to or used for a closely related species such as a human or a baboon in the same or a similar or comparable clinical situation. Determination of a similar or comparable clinical situation can be based, e.g., on a clinician's or veterinarian's judgment and/or measurement of one or more biological parameters in or from the subject. In some cases, the known status profile will be based on a biological insult and/or biological responses that are the essentially the same or similar to those used for the species where the status profile is not as well characterized or is unknown. Status profiles can thus be obtained for (i) an individual subject, (ii) exposed treated subjects or groups of subjects and/or (iii) exposed treated individual subjects or groups and/or (iv) an individual or subject that is of the same species or a closely related species that has received the same biological insult, e.g., radiation or chemotherapy dose, essentially the same biological insult or a similar or otherwise comparable biological insult and/or (vi) an individual subject or exposed treated individual subjects or groups of subjects that is/are of the same species or a closely related species that has received the same biological insult, essentially the same biological insult or a similar or otherwise comparable biological insult.

As noted above, the status profile can be established so as to predict either lethality or death (P_{lethality}) or survival (Pₛᵤᵣᵥᵢᵥₐₗ) with a defined probability. Several statistical methods can be used to calculate the probability for the status profile. These methods include use of one-way, two-way, two-way repeated-measures ANOVA (with day and time-of-day as the repeated measures), spectral analysis, generalized linear models, generalized linear and non-linear mixed models and/or autogressive moving average models. In some cases, such models can describe or capture the essence of a subject's past profile data and thus they can be used to project and forecast the evolution of the profile to collapse of an exposed subject's immune system or to survival of the subject.

As is apparent from the foregoing discussion, depending on the number of subjects and the statistical method that is used, the value of P_{lethality} or Pₛᵤᵣᵥᵢᵥₐₗ can vary from levels that are not remarkable to levels that are highly deterministic, e.g., P_{lethality} or Pₛᵤᵣᵥᵢᵥₐₗ is about 0.15 or about 0.1 to levels that are typically considered statistically significant (i.e., statistically significantly not zero), e.g., P is at least 0.5, or highly significant, e.g., P is at least about 0.9 or at least about 0.95. Knowledge of the P allows the clinician to tailor any clinical or therapeutic treatment to the subject's clinical condition.

In some embodiments, the status profile comprises temperature or temperature profile and optionally one, two or more of red blood cell count, blood hematocrit, blood reticulocyte count, relative reticulocyte count, blood platelet count, blood megakaryocyte count from one or more exposed subjects or groups of exposed subjects, or one or more exposed treated subjects or groups of exposed treated subjects. In other embodiments, (i) the status profile comprises red blood cell count, blood hematocrit, relative reticulocyte count or blood reticulocyte count, and optionally one, two or more of temperature or temperature profile, blood platelet count or blood megakaryocyte count from one or more exposed subjects or groups of exposed subjects, or one or more exposed treated subjects or groups of exposed treated subjects, or (ii) the status profile comprises blood platelet count or blood megakaryocyte count and optionally one, two or more of temperature or temperature profile, red blood cell count, blood hematocrit, relative reticulocyte count or blood reticulocyte count from one or more exposed subjects or groups of exposed subjects, or one or more exposed treated subjects or groups of exposed treated subjects, or (iii) the status profile comprises neutrophil count, white blood cell count or absolute white blood cell differential, and optionally one, two or more of temperature or temperature profile, red blood cell count, blood hematocrit, blood reticulocyte count blood platelet count or blood megakaryocyte count from one or more exposed subjects or groups of exposed subjects, or one or more exposed treated subjects or groups, (vi) the status profile comprises one or two biological parameters described herein, and optionally one, two or more of neutrophil count, white blood cell count or absolute white blood cell differential, temperature or temperature profile, red blood cell count, blood hematocrit, blood reticulocyte count blood platelet count or blood megakaryocyte count from one or more exposed subjects or groups of exposed subjects, or one or more exposed treated subjects or groups.

As is apparent from the foregoing discussion, reference to a status profile that comprises one or more biological parameters described herein, e.g., temperature, circadian rhythm, blood pressure, hematocrit, red cell count, neutrophil count and/or platelet counts, means that the subject(s) status profile is based on one or more measurements of that parameter. Typically, most of these measurements are at a time after the biological insult when the biological parameter is changed, i.e., detectably increased or decreased, which may be a statistically significant change or not, from baseline or the exposed subject(s) or for one or more reference subjects of the same or a closely related species that have been exposed to at the same or a similar or comparable biological insult and where a status profile has previously been established for the closely related species.

As used herein, the phrase ‘closely related species’ generally refers to species or subspecies (i) that are in the same Order or Family, usually in the same Genus, and/or (ii) wherein the subjects share at least about 90%, at least about 95%, at least about 98% or at least about 99% homology for 1, 2, 3, 4, 5, 6 or more genes that are considered reasonable or reliable indicators of taxonomic relatedness for species in a given Phylum, Class, Order, Family or Genus, e.g., cytochrome, immunoglobulin, enzyme or cell surface molecule. In general, humans and most non-human primates are closely related species and thus a status profile for a non-human primate such as Rhesus monkey (Macaca mullata), Cynomolgus monkey (Macaca fascicularis), Japanese monkey (Macaca fuscata), African Green monkey, pig-tailed macaque, marmoset, cotton top tamarin, talapoin, squirrel monkey, or a baboon such as the olive baboon, that is based on a biological insult or biological parameters described herein is a suitable reference for a human that has been exposed to the same or a similar biological insult. It will be appreciated that in some cases, a status profile for a human may be obtained for exposed treated individuals, since medical care standards dictate that persons receiving biological insults that are potentially life-threatening, e.g., high dose radiotherapy or high dose cancer or glucocorticoid chemotherapy, also usually or always receive other ameliorative, palliative treatments such as antibiotic treatments or platelet transfusions. In other cases, reference to a status profile from a closely related species can be used, including in situations where the status profile is based on exposed subjects, e.g., non-human primates, that are not exposed treated subjects.

In any of these embodiments, measurements of any of the biological parameters can be obtained on one or more occasions, but typically a given parameter will be measured on 2, 3, 4, 5, 6, 7, 8, 9, 10, 11, 12, 13, 14, 15, 16, 17, 18, 19, 20, 21, 22, 25, 30, 40, 50, 60, 100, 200, 300, 400, 500 or more occasions, any of which measurements may be begun before, during or after the subject or subjects have been or will be exposed to the biological insult. Measurements of biological parameters described herein, e.g., mature or immature blood cell types, will typically be obtained at intervals of at least about 6 hours, at least about 12 hours, at least about 1 day, at least about 1.5 days, at least about 2 days, at least about 3 days and/or at least about 4 days, usually on two, three, four or more occasions. When biological parameters, e.g., one, two or more of temperature, heart rate, SaO₂, blood pressure, or a parameter that can be measured continuously or measured by suitable apparatus, are measured on many occasions, e.g., on about 15, about 20 or more occasions, the biological parameter can be monitored continuously, which can optionally be monitored in real time. When the temperature is measured on 4 or more occasions, the measurements can be obtained on a periodic basis, optionally in real time, optionally wherein the real time temperature measurements are obtained at intervals of about 1 minute or about 2 minutes to about 5 minutes, about 10 minutes or about 20 minutes.

When measurement of core body or peripheral temperature is taken to determine if the subject's circadian rhythm has been significantly disrupted, e.g., when the normal daily temperature fluctuations associated with the subject species has been completely or at least partially obscured as observed by sufficient temperature measurements to reliably detect disruption. Temperature measurements can be oral, axillary, rectal, tympanic, skin, rectal or from an implanted or ingested device for core temperature. Temperature measurements can be measured intermittently and/or continuously, e.g., on intervals of about 0.1-30 minutes or about 0.5-10 minutes, or periodically at 1, 2, 3, 4, 5, 6, 7, 8, 9 or more times in a 1, 2 or 3 day period when a characteristic temperature associated with the normal circadian rhythm is expected. Usually core body temperature will be measured to assess circadian rhythm. Other means to assess disruption of circadian rhythm can also be used. Circadian rhythm can be assessed over a 48 hour or 72 hour period using a Mini-Motionlogger Actigraph (Ambulatory Monitoring, Ardsley, N.Y.), starting within 1, 2 3, 4, 5, 6 or more days after the biological insult. For biological insults such as cancer chemotherapy, monitoring will begin at about 5, 6 or 7 days after administration of the chemotherapy agent. For biological insults such as radiation exposure, monitoring will begin at about 2 hours or about 6 hours to about 1 or 2 days after the exposure. Daily patterns of sleep and activity can be compared across the monitoring period using autocorrelation analyses to calculate a circadian rhythm score for each subject, with higher scores associated with lower disruption. Comparisons of fatigue, depression and/or mood with subject circadian rhythm measures taken after the biological insult. Changes in fatigue, depression and mood measures are compared with concurrent changes in circadian rhythm. Other parameters or analyses that can be measured on one or more occasions or used to assess circadian rhythm and its disruption include (i) measuring elevated or decreased cortisol or IL-6 at about 9:00 a.m. to about 12:00 p.m. on 1, 2, 3, 4 or more days (elevated human blood cortisol is about 32±5 μg/dL of blood and normal human blood cortisol is about 18±7 μg/dL of blood), (ii) variations in skin temperature or skin blood flow using, e.g., laser Doppler imaging or a skin thermometer over a about 24 hours, 28 hours, about 48 hours or linger, (iii) casino analysis to estimate circadian rhythm meson, amplitude or atrophies, (iv) salivary or blood endothelia or melatonin levels, (v) theta, sigma and/or delta sleep brain wave patterns, (vi) blood C reactive protein or fibrinogen level, and/or (vii) circulating DHEA levels. Methods to assess the circadian rhythm and its disruption have been described and they can be applied in the present methods, see, e.g., J. A. Roscoe et al., Support Care Cancer. 10(4):329-36, 2002, P. Fantidis et al., Eur. J. Clin. Invest. 32:304-308 2002, G. Yosipovitch et al., J. Invest. Dermatol. 122:824-829 2004, K. A. Thomas et al., Biol. Res. Nurs. 5:187-194 2004, S. Xiang et al., Clin. Chem. 49:2012-2019 2003, C. J. van den Heuvel et al., Physiol. Meas. 24:717-725 2003, and X. Tan et al., Neurosci. Lett. 344:205-208 2003.

Core body temperature or peripheral temperature can be obtained using an implanted device, which can be surgically implanted, taken orally or using a device such as a thermister in an indwelling catheter, central venous catheter or other line that is in a artery or vein in a subject or core body temperatures can be obtained by measuring rectal temperature for all or at least a part of the time period when temperature is being monitored. When a temperature measuring device is used in an indwelling line, other devices may also be used to measure one or more other biological parameters such as blood pressure, blood oxygen levels, blood pH or electrolyte composition, any of which can be periodically measured, e.g., once per minute, once per 5 minutes or once per 10 minutes, any of which measurements are optionally taken on a real time basis. Temperature is optionally measured on 4 or more occasions or is measured on a periodic basis, optionally in real time, optionally wherein the real time temperature measurements are obtained at intervals of about 1 minute to about 60 minutes, e.g., at about 5 minute, about 10 minute, about 15 minute or about 20 minute intervals.

For some biological parameters, e.g., neutrophil counts, red cell counts, hematocrit, platelet counts, sepsis or a temperature drop associated with sepsis, a relatively small number of measurements can typically be used to obtain a status profile, e.g., about 1, 2, 3, 4, 5, 6, 7 or 8 measurements are obtained. For any of these parameters or for survival of the subjects, measurements or observations are optionally made over a period of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 35, 40, 45, 50, 55, 60 or more days. Any biological parameter described herein can be measured on a few occasions or on many occasions, where this is practical or possible under the circumstances as is apparent to one of ordinary skill in the art.

For measuring blood cells or precursors or markers or other biological parameters that usually at least transiently decrease or that can be are disrupted after the biological insult, e.g., one or more elements, biomolecules or biological parameters that vary on a circadian rhythm, and/or one or two of the nadir or lowest value(s) for that parameter will usually be used in the calculation of the exposed subject(s)' status profile. For measuring temperature, heart rate or other biological parameters that usually at least transiently increase or are disrupted, e.g., circadian rhythm or an element thereof, after the biological insult, one or two of the peak or high value(s) for that parameter or for the disruption will usually be used in the calculation of the exposed subject(s)' status profile. When a relatively small number of measurements are anticipated or are only practical, the measurements will typically be timed, where possible, to coincide with time(s) when the parameter is the most informative in terms of adding statistical power to the status profile. Thus, for decreases or other changes in blood cells or components such as red cells, reticulocytes, platelets, megakaryocytes, neutrophils or other biological parameters described herein in humans or non-human primates these measurements will be close to or within the time period when a nadir for that parameter would be expected, e.g., on one, two or more occasions at about 12, 13, 14, 15, 16, 17, 20 18, 19 or 20 days after exposure to radiation or a myelosuppressive or cytotoxic cancer chemotherapy. Similarly, these measurements in humans or non-human primates will be close to or within the time period when a peak or maximum for a parameter such as temperature or the degree of disruption of the circadian rhythm or an element thereof, would be expected, e.g., on one, two or more occasions at about 1, 2, 3, 4 or 5 days after exposure to radiation, a myelosuppressive or cytotoxic cancer chemotherapy or a serious trauma, e.g., hemorrhagic trauma. Biological parameters in humans or non-human primates such as sepsis, pain, fatigue, heart rate, hypotension or hypertension, are expected to peak or have a maximum change for baseline at about 6, 7, 8, 9, 10, 11, 12, 13, 14, 15, 16, 17, 18, 19, 20 or 21 days. Once a status profile is known or a given type of biological insult, the number and time of parameter measurements can be targeted to the times that are the most informative under the circumstances.

When two or more biological parameters are used to obtain the subject's status profile, measurements of each parameter can be initiated at about the same time, essentially the same time or at different times. However, measurements of each parameter, or preparation to measure each parameter, will typically begin (i) at about the same time, e.g., within about 10-30 minutes or within 1 or 2 hours of each other or (ii) at essentially the same time, e.g., measurements of each biological parameter, or preparation to measure each parameter, are initiated on the same day, usually within about 2.5 hours or about 3 hours to about 4 hours or about 6 hours. In some embodiments, most, e.g., at least about 60%, at least about 70%, at least about 75%, at least about 80%, at least about 90% or all, of these measurements will occur beginning after the subject(s) has been exposed to the biological insult.

The subject in the methods can be a non-human primate, a human, a rodent, a lagomorph, a canine, a feline, a myomorph, a lagomorph, a chiropteran, an artiodactyl or porcine, a carnivore, a rodent or another type of subject described herein.

To obtain data for some of these methods, a F1C can be administered to a subject or group of subjects. This can comprise administering to a subject exposed to a biological insult, or delivering to the exposed subject's tissues, an effective amount of any F1C compound or structure disclosed herein.

Specific exemplary status profiles include status profiles that are based on measuring the following combinations of biological parameters, which are measured on one or more occasions:

(i) a temperature increase (or a measure of central tendency) of at least about 0.5° C., at least about 0.6° C., at least about 0.7° C., at least about 0.8° C., at least about 0.9° C., at least about 1.0° C., at least about 1.1° C., at least about 1.2° C., at least about 1.3° C., at least about 1.4° C., at least about 1.5° C., at least about 1.6° C., at least about 1.7° C., at least about 1.8° C., at least about 1.9° C., at least about 2.0° C., at least about 2.1° C. or at least about 2.3° C. above the baseline of the normal temperature for the subject species, e.g., about 37.2° C. for Rhesus monkeys or about 98.6° F. for humans, optionally where the temperature increase optionally is (a) completely or mostly (at least about 80% or at least about 90% or at least about 95% or at least about 98% of the time) maintained at that level for a period of at least about 0.5 minute, at least about 1 minute, at least about 5 minutes, at least about 10 minutes, at least about 0.25 hour, at least about 0.5 hour at least about 0.75 hour, at least about 1 hour or at least about 2 hours, at least about 3 hours, at least about 4 hours or at least about 6 hours, or at least about 8 hours, optionally where the temperature increase occurs within about 4 hours, about 8 hours, about 12 hours, about 24 hours or about 48 hours of the biological insult, and/or (b) when the subject is a human or a non-human primate, core or peripheral temperature is measured within a period of about 1, 2, 3, 4, 5, 6, 7, 8, 9, 10, 11, 12, 13, 14, 15, 16, 17, 18, 19, 20 or 21 days after the biological insult and/or (c) the core body temperature is measured, e.g., using rectal temperature, an implanted monitoring device and/or a thermister in an indwelling catheter or line, and/or (d) the status profile correlates with lethality of the biological insult for the exposed subject, or status profile correlates with survival after the biological insult for the exposed subject when the temperature increase is not observed;

(ii) disruption of the circadian rhythm as described or defined as, e.g., a significant change in the normal rhythm or signature in any of the elements of the composite of a circadian rhythm such as temperature, in the subject species, optionally where the disruption is (a) completely or mostly (at least about 80% or at least about 90% or at least about 95% or at least about 98% of the time) maintained for a period of at least about 1 hour or at least about 2 hours, at least about 3 hours, at least about 4 hours or at least about 6 hours, at least about 8 hours, at least about 12 hours, at least about 24 hours or at least about 48 hours, and/or (b) when the subject is a human or a non-human primate, core or peripheral temperature is measured within a period of about 1, 2, 3, 4, 5, 6, 7, 8, 9, 10, 11, 12, 13, 14, 15, 16, 17, 18, 19, 20, 21, 22, 23 or 24 days after the biological insult and/or (c) the status profile correlates with lethality of the biological insult for the exposed subject when the circadian rhythm is completely or mostly disrupted or the status profile correlates with survival after the biological insult for the exposed subject when the circadian rhythm is not completely or mostly disrupted;

(iii) a mean or absolute decrease (or a measure of central tendency) of at least about 20%, at least about 21 %, at least about 22%, at least about 23%, at least about 24%, at least about 25%, at least about 26%, at least about 27% or at least about 28%, at least about 29% or at least about 30%, in red blood cell or erythrocyte counts, hematocrit, hemoglobin and/or reticulocytes, optionally where (a) the mean decrease in red blood cell or erythrocyte counts, hematocrit, hemoglobin and/or reticulocytes is obtained from the nadir or lowest measurement, optionally, and/or (b) when the subject is a human or a non-human primate, the red blood cell or erythrocyte count, hematocrit, hemoglobin and/or reticulocyte count is measured within a period of about 7, 8, 9, 10, 11, 12, 13, 14, 15, 16, 17, 18, 19, 20, 21, 22, 23 or 24 days after the biological insult and/or (c) the status profile correlates with lethality of the biological insult for the exposed subject, and/or (d) temperature variation or increase or circadian rhythm disruption is also measured, e.g., as described in (i), (ii) or elsewhere herein;

(vi) an absolute decrease of at least about 78%, about 79% or about 80% or about 85% in red blood cell or erythrocyte counts, hematocrit, hemoglobin and/or reticulocytes for individual exposed subjects or for groups of exposed subjects, optionally where (a) the mean decrease in red blood cell or erythrocyte counts, hematocrit, hemoglobin and/or reticulocytes is obtained from the nadir or lowest measurement, optionally, and/or (b) when the subject is a human or a non-human primate, the red blood cell or erythrocyte count, hematocrit, hemoglobin and/or reticulocyte count is measured within a period of about 7, 8, 9, 10, 11, 12, 13, 14, 15, 16, 17, 18, 19, 20, 21, 22, 23 or 24 day$ after the biological insult and/or (c) the status profile correlates with survival of the exposed subject after the biological insult, and/or (d) temperature variation or increase or circadian rhythm disruption is also measured, e.g., as described in (i), (ii) or elsewhere herein;

(v) an absolute decrease of at least about 80%, at least about 85%, at least about 90%, at least about 95%, at least about 97%, in platelets, megakaryocytes or a megakaryocyte precursor described herein, or, for a human or a non-human primate, a mean count of about 6500 per μL or less, about 6600 per μL or less, about 6700 per μL or less, about 6800 per μL or less, about 6900 per μL or less or about 7000 per μL or less for non-human primates or humans or about 10,000 per μL or less, about 9,500 per μL or less, about 9,000 per μL or less, about 8,500 per μL or less or about 8,000 per μL or less, optionally where (a) the mean decrease in platelets, megakaryocytes or megakaryocyte precursors is obtained from the nadir or lowest measurement, and/or (b) when the subject is a human or a non-human primate, the platelet, megakaryocyte or megakaryocyte precursor count is measured within a period of about 6, 7, 8, 9, 10, 11, 12, 13, 14, 15, 16, 17, 18, 19, 20, 21, 22, 23 or 24 days after the biological insult and/or (c) the status profile correlates with lethality of the biological insult for the exposed subject, and/or (d) temperature variation or increase or circadian rhythm disruption is also measured, e.g., as described in (i), (ii) or elsewhere herein;

(vi) an absolute decrease of less than about 78%, less than about 75%, less than about 70% or less than about 65%, in platelets, megakaryocytes or megakaryocyte precursors, optionally where (a) the mean decrease in platelets, megakaryocytes or a megakaryocyte precursor described herein is obtained from the nadir or lowest measurement, and/or (b) when the subject is a human or a non-human primate, the platelet, megakaryocyte or megakaryocyte precursor count is measured within a period of about 6, 7, 8, 9, 10, 11, 12, 13, 14, 15, 16, 17, 18, 19, 20, 21, 22, 23 or 24 days after the biological insult and/or (c) the status profile correlates with survival of the exposed subject after the biological insult, and/or (d) temperature variation or increase or circadian rhythm disruption is also measured, e.g., as described in (i), (ii) or elsewhere herein;

(vii) an absolute decrease of at least about 80%, at least about 85%, at least about 90%, at least about 95%, at least about 97%, in neutrophils or a neutrophil precursor described herein, and/or, for a human or a non-human primate, an absolute count of about 30 per mm³ or less, about 40 per mm³ or less, about 45 per mm³ or less, about 50 per mm³ or less or about 55 per mm³ or less, optionally where (a) the mean decrease in neutrophil or neutrophil precursor is obtained from the nadir or lowest measurement, and/or (b) when the subject is a human or a non-human primate, the neutrophil or neutrophil precursor count is measured within a period of about 7, 8, 9, 10, 11, 12, 13, 14, 15, 16, 17, 18, 19, 20, 21, 22, 23 or 24 days after the biological insult and/or (c) the status profile correlates with lethality of the biological insult for the exposed subject, and/or (d) temperature variation or increase or circadian rhythm disruption is also measured, e.g., as described in (i), (ii) or elsewhere herein, and/or (e) a decrease in one or more of platelets, megakaryocytes or another thrombopoiesis marker as described in (v) or (vi) or elsewhere herein and/or (f) a decrease in one or more of red cell counts or hematocrit or other erythropoiesis marker as described in (iii) or (iv) or elsewhere herein;

(viii) an absolute decrease of less than about 78%, less than about 75%, less than about 70% or less than about 65%, in neutrophils or in a neutrophil precursor described herein, or, for a human or a non-human primate, a mean count of at least about 50 per mm³, at least about 55 per mm³, at least about 60 per mm³, at least about 65 per mm³, at least about 70 per mm³, at least about 80 per mm³, at least about 90 per mm³, at least about 100 per mm³, at least about 150 per mm³, at least about 200 per mm³, at least about 300 per mm³ or at least about 400 per mm³, optionally where (a) the mean decrease in neutrophils or neutrophil precursor is obtained from the nadir or lowest measurement, and/or (b) when the subject is a human or a non-human primate, the neutrophil or a neutrophil precursor count is measured within a period of about 7, 8, 9, 10, 11, 12, 13, 14, 15, 16, 17, 18, 19, 20, 21, 22, 23 or 24 days after the biological insult and/or (c) the status profile correlates with survival of the exposed subject after the biological insult, and/or (d) temperature variation or increase or circadian rhythm disruption is also measured, e.g., as described in (i), (ii) or elsewhere herein, and/or (e) a decrease in one or more of platelets, megakaryocytes or another thrombopoiesis marker as described in (v) or (vi) or elsewhere herein and/or (f) a decrease in one or more of red cell counts or hematocrit or other erythropoiesis marker as described in (iii) or (iv) or elsewhere herein;

(ix) the first time after the biological insult that the exposed subject, usually a human or a non-human primate, has a Grade III or IV thrombocytopenia or an equivalent condition, e.g., a platelet count of less than 50,000 per mm³, optionally combined with one or more of the biological parameters described in (i), (ii), (iii), (iv), (v), (vi), (vii) or (viii) above or one, two or more biological parameters described elsewhere herein;

(x) the first time after the biological insult that the exposed subject, usually a human or a non-human primate, has a Grade III or IV anemia or an equivalent condition, e.g., hemoglobin measurement of less than 8.0 g per dL, optionally combined with one or more of the biological parameters described in (i), (ii), (iii), (iv), (v), (vi), (vii), (viii) or (ix) above or one, two or more biological parameters described elsewhere herein; and/or

(xi) the status profile of any of (i), (ii), (iii), (iv), (v), (vi), (vii), (viii), (ix) or (x) wherein (a) the subject is a treated exposed subject and the treatment optionally is one, two or more of administration of an effective amount of a hematopoiesis stimulator, an immune system stimulator, an apoptosis inhibitor, an antibiotic, an antifever treatment or agent, an analgesic, whole blood, platelets, red cells, neutrophils, electrolytes, anti-fever agents, analgesics, G-CSF, GM-CSF, IL-6, IL-11, IFNγ, intravenous fluids, intravenous immunoglobulin, intravenous nutrients or sugars, anti-TNF-α antibody or monoclonal antibody or antibody fragment, thrombopoietin, erythropoietin, stem cell factor, pegfilgrastim, α-1 thymosin, thymopoietin, serum thymic factor, an antioxidant, a CpG oligonucleotide, allopurinol, vitamin E or related compounds, superoxide dismutase mimetics, a benzyl styryl sulfone, dipeptide peptidase inhibitors, phenylacetic acid, phenylbutyric acid, an apoptosis inhibitor or hematopoiesis stimulator optionally selected from a steroid of formula 1, a bacterial flagellin and an antiapoptotic fragment thereof, a biologically active fragment of any of these proteins, a polymer conjugate of any of these proteins or any biologically active fragment of any of these proteins, a statin, e.g., as described herein or in the cited references, a F1C, and/or (b) the subject is a human or a non-human primate, optionally selected from a Rhesus monkey and a Cynomolgus monkey, and/or (c) the status profile is obtained from exposed subjects, exposed treated subjects and/or both exposed subjects and exposed treated subjects, and/or (d) the biological insult is radiation exposure, optionally at a dose of about an LD₃₀ or LD₄₀ or LD₄₅ to about an LD₅₅, LD₆₀ or LD₇₀ or at a dose of about an LD₅₀, or at another dose or dose range described herein, where survival is determined at 30 days post exposure or at 60 days post exposure, and optionally where the radiation is y-radiation such as ⁶⁰Co or ¹²⁷Cs, particle radiation, e.g., silicon or boron, fast neutrons or slow neutrons, and optionally wherein the radiation is whole body radiation that the subject(s) is exposed to over a period of about 30 minutes or less or about 20 minutes or less or where the subject(s) is exposed to the radiation for a period of about 10±3 minutes, and optionally where a treatment agent selected from administration of an effective amount of a steroid of formula 1, IL-6, IFNγ, G-CSF, GM-CSF or another treatment described herein is administered to the subject, optionally where the administration results in the treatment agent being systemically present in the subject at 1, 2 or more times within about 0.5 hours, about 1 hour, about 1.5 hours, about 2 hours, about 2.5 hours about 3 hours about 3.5 hours, about 4 hours, about 4.5 hours, about 5 hours, about 5.5 hours, about 6 hours, about 8 hours, about 12 hours or about 24 hours after the subject was exposed to the radiation; and/or (e) the biological insult is radiation exposure, optionally at a dose of about 450 cGy, about 500 cGy, about 550 cGy, about 560 cGy, about 570 cGy, about 580 cGy, about 590 cGy, about 600 cGy, about 610 cGy, about 620 cGy, about 630 cGy, about 640 cGy, about 650 cGy, about 700 cGy, about 750 cGy, about 800 cGy, about 850 cGy, about 9 Gy, about 9.5 Gy, about 10 Gy, about 10.5 Gy, about 11 Gy, about 12 Gy, about 15 Gy, about 20 Gy or another radiation dose or dose range described herein, optionally wherein the radiation is whole body radiation that the subject(s) is exposed to over a period of about 30 minutes or less or about 20 minutes or less or where the subject(s) is exposed to the radiation for a period of about 10±3 minutes and optionally where the radiation is a radiation disclosed herein, e.g., γ-radiation such as ⁶⁰Co or ¹²⁷Cs or fast neutrons, and optionally where a treatment agent selected from administration of an effective amount of a steroid of formula 1, IL-6, IFNγ, G-CSF, GM-CSF, thrombopoietin, erythropoietin or another treatment described herein is administered to the subject, optionally where the administration results in the treatment agent being systemically present in the subject at 1, 2 or more times within about 0.5 hours, about 1 hour, about 1.5 hours, about 2 hours, about 2.5 hours about 3 hours about 3.5 hours, about 4 hours, about 4.5 hours, about 5 hours, about 5.5 hours, about 6 hours, about 8 hours, about 12 hours or about 24 hours after the subject was exposed to the radiation; and/or (f) the biological insult is 1, 2, 3, 4, 5, 6 or more rounds of 1, 2, 3, 4 or more cancer chemotherapies or cancer chemotherapy agents or a bone marrow transplantation protocol, or a surgery, any of which are optionally combined with radiation exposure, optionally wherein the biological insult occurs over a time period of about 1, 2, 3, 4, 5, 6, 7, 8, 9, 10, 11, 12, 13, 14, 15, 16, 17, 18, 19, 20 or more days or over a time period of about 3, 4, 5, 6, 7, 8, 9, 10, 11, 12, 13, 14, 15, 16, 17, 18, 19 or more weeks or aver a time period of about 5, 6, 7, 8, 8, 10, 11, 12 or more months, optionally wherein one, two or more biological parameter measurements to obtain the status profile are begun at about at time when the subject(s) would be expected to have a significant chance (P>about 0.1, about 0.2, about 0.3, about 0.4, about 0.5, about 0.6 or more) of not surviving the biological insult, where the assessment of the significant chance of not surviving the biological insult is a subjective or objective assessment based on clinical observations and/or comparison of the subject(s)' condition with similarly situated subjects of the same or a closely related species, optionally wherein, for a subject(s) that has a cancer, the cancer is optionally selected from lung cancer, prostate cancer, breast cancer, colon cancer, skin cancer, a cancer of the central or peripheral nervous system, ovarian cancer, cervical cancer and endometrial cancer.

In general, a significant change in the normal rhythm or signature in any of the elements of the composite of a circadian rhythm can occur as a biological response to the biological insult. A significant change is generally a change that is statistically significantly not zero, e.g., P<0.05, or a change that is nearly statistically significantly not zero, e.g., P<0.15, P<0.12 or P<0.10. A change(s) can be observed in one, two, three or more of the elements of the composite of a circadian rhythm, e.g., circadian sex steroid levels, cortisol, IL-6, melatonin or other molecules described herein. Change in circadian temperature can be observed as a relatively flat daily temperature profile, intermittent changes, e.g., hectic fever, as significant shifts in cycle timing, exaggerated temperature peaks and/or valleys or as combinations of these situations.

Any of the invention methods disclosed herein are optionally combined with one or more other treatments. In some embodiments, guanylyl cyclase or cGMP synthesis enhancers such as substance P, substance P analogs, substance P mimetics or nitroglycerin are administered alone or in combination with a F1C treatment. Guanylyl cyclases that can be modulated, e.g., at least transiently enhanced, include soluble NO-activated guanylyl cyclase in neurons or other CNS cells, epithelial cells, endothelial cells, monocytes, macrophages, neutrophils, other white blood cells and/or muscle cells or myocytes. In some embodiments, one or more of these treatments are used without administering any F1C, e.g., to characterize the capacity of the therapeutic agent to affect one, two or more biological responses to a biological insult, e.g., radiation exposure, or as a therapy that is optionally combined with a blood or blood fraction transfusion and/or one or more antimicrobial therapies, e.g., to prevent or treat a bacterial, fungal or viral infection that may be caused by or exacerbated by a biological insult.

Definition of clinical conditions such as Grade II, III or IV fever, fatigue, weight loss, pain, thrombocytopenia, neutropenia, anemia, hypotension, hypertension, hypoxia, skin burn, rash, skin ulceration, anorexia, colitis, dehydration, diarrhea, distension, enteritis, mucositis, nausea, necrosis, vomiting, hemorrhage, petechiae, pancreatitis, febrile neutropenia, colitis, infection, head or neck edema, limb edema, edema of the edema, alkalosis, acidosis, hypocalcemia, creatine phosphokinase, bone fracture, myositis, cerebrovascular ischemia, confusion or other clinical conditions described herein is as described elsewhere herein and/or as described in the common terminology criteria for adverse events v3.0, which is published at http://ctep.cancer.gov, with current version published on Dec. 12, 2003. The biological insult can give rise to a range of biological responses or clinical conditions, which include one or more of these defined clinical conditions, some of which may arise soon after exposure to the biological insult, e.g., within about 10 minutes to about 24 hours.

Use of the status profile for diagnosis, treatment selection and other analyses. In some embodiments, the invention provides methods to determine a status profile for a subject species comprising, (1) exposing a sufficient number of subjects to a biological insult of at least about an LD_{10/60} to obtain exposed treated subjects; (2) measuring on two or more occasions in or from the exposed subjects one, two or more biological parameters selected from body temperature, circadian rhythm, red blood cell counts, hematocrit, reticulocytes, platelets, megakaryocytes and neutrophils; (3) measuring or modeling the survival rate or experience of the exposed subjects; (4) obtaining one or more status profiles that corresponds to a defined probability of surviving the biological insult (Pₛᵤᵣᵥᵢᵥₐₗ) of at least 0.95 or of not surviving the biological insult (P_{lethality}) of at most 0.05; and (5) optionally using the status profile to identify and initiate a profile-based therapy for one or more of the exposed subjects.

In these embodiments, the biological insult can be a radiation or other exposure of about an LD_{30/60} to about an LD_{70/60} or wherein the biological insult is about an LD_{50/60}. Biological parameters or responses that can be measured include one or more of circadian rhythm, hematocrit, platelets, temperature, e.g., core body temperature, which is optionally measured (i) using an implanted monitor, and/or (ii) continuously and/or (iii) at intervals of about 1 minute, about 5 minutes or about 10 minutes to about 30 minutes, about 1 hour or about 2 hours, or another biological parameter disclosed herein.

Other aspects of the invention and related subject matter center on methods to obtain a status profile having a defined Pₛᵤᵣᵥᵢᵥₐₗ or P_{lethality} for an exposed subject(s) and/or to a previously established status profile, e.g., to diagnose or characterize the clinical status of the exposed subject(s) and/or to identify appropriate treatments for the exposed subject(s). In these embodiments, the status profile is usually obtained from (i) the exposed subject himself or herself, and optionally compared to a suitable comparable status profile(s) from one or more subjects of the same or a closely related species where the biological insult and biological parameters are the same or essentially the same or are otherwise comparable. Related aspects of the invention include comparison of one or more status profiles having a defined Pₛᵤᵣᵥᵢᵥₐₗ or P_{lethality} from exposed subjects, with a similarly based status profile from a closely related species and/or a species that is not closely related. Such comparisons provide a means, e.g., to compare physiology between different species and/or to diagnose the clinical condition of exposed subject(s).

Additional embodiments include the use of a status profile(s) having a defined Pₛᵤᵣᵥᵢᵥₐₗ or P_{lethality} from an exposed subject(s) in a submission or report. Such submissions or reports include the use of the status profile in a grant application, an oral or written scientific presentation or publication or in an oral or written regulatory report or submission, e.g., to the U.S. Food and Drug Administration or a foreign counterpart medical or food regulatory agency, the U.S. Environmental Protection Agency, the U.S. Department of Defense, the U.S. Department of Energy, the U.S. Department of Health and Human Services, the U.S. National Institutes of Health or a foreign counterpart medical, health, environmental or defense agency or to another U.S. domestic or foreign regulatory agency, any Institutional Animal Care and Use Committee, or any U.S. or foreign local, state or federal government, where such report or submission is optionally required under any applicable law, statute, rule, regulation or any other requirement, e.g., as provided under any statute, rule or amendment in title 21 of the U.S. Code of Federal Regulations title 35 of the United States Code, e.g., at one or more rules or statutes at one or more of 21 C.F.R. Part 58, 35 U.S.C. § 101,35 U.S.C. § 271(e), 35 U.S.C. § 112, e.g., at paragraph 1, 2 or 6 of § 112, at one or more portions of the U.S. Food Drug and Cosmetic Act such as at § 505()(2)(A), 21 U.S.C. §§ 301 et. seq., 21 U.S.C. § 355(j)(2) or Section 515 of the Federal Food, Drug, and Cosmetic Act, 90 Stat. 552, 21 U.S.C. § 360e, 21 U.S.C. § 355(j)(2)(A)(vii)(l)-(IV), 21 U.S.C. § 355(j) (2)(B), 21 U.S.C. § 351, 21 U.S.C. § 352, 21 U.S.C. § 353, 21 C.F.R. § 314, 21 C.F.R. §§ 314, 314.600, 314.610, 314.620, 314.630, 21 C.F.R. § 600, 21 C.F.R. §§ 601, 601.90, 601.91, 601.92, 601.93.

In some of these embodiments, the invention provides methods comprising, (1) providing or obtaining a subject who has been exposed to a biological insult; (2) measuring one, two or more of the subject's biological responses to the biological insult to obtain the subject's status profile with a defined Pₛᵤᵣᵥᵢᵥₐₗ or P_{lethality}; (3) optionally initiating the one or more palliative therapies at a time before, during or after the determination of the status profile; (4) using the subject's status profile to identify one or more profile-based therapies; (5) optionally administering one or more profile-based therapies to the subject; and (6) optionally maintaining at least one of the one or more palliative and/or profile-based therapies until the subject has sufficiently recovered from the biological insult to have an improved probability of surviving the biological insult or has an improved clinical condition or prognosis or until the subject has mostly or fully recovered from the biological insult. In these embodiments, the palliative therapies are typically dissimilar from the profile-based therapies. For these methods, the biological insult is as described herein, e.g., exposure to radiation, a chemotherapy or another biological insult described herein where the exposure of the exposed subjects or species has a significant probability of causing a potentially life-threatening side effect or biological response that would be expected to be at least about an LD_{0.1}, at least about an LD_{0.5}, at least about an LD₅ or another degree of LD described elsewhere herein.

Biological responses or biological parameters that can be measured before and/or after the biological insult include temperature. Core body temperature using or peripheral temperature can be measured using, e.g., one or more methods described herein such as rectal temperature measurements, oral temperature measurements, an implanted temperature monitoring device or a thermister, e.g., in a catheter or attached to the skin.

Typical palliative therapies include the administration or use of one, two or more of fluids, e.g., for dehydration, electrolytes, analgesics, anti-nausea or anti-emesis agents such as decadron, anti-hypotension agents, agents for respiratory distress, treatment for hypothermia, e.g., for special populations, sleep enhancing agents, fever control, nutritional control or supplementation. In general, profile-based therapies will comprise one or more treatments that (i) modulate or reduce one or more of the adverse biological responses to the biological insult and/or that (ii) enhance the recovery of damaged cell or tissues, particularly for normal or non-pathological cells or tissues and/or (iii) reduce the degree or severity of damage, particularly for normal or non-pathological cells or tissues. As is apparent to one of ordinary skill in the art, in some cases palliative and profile-based therapies will at least partially overlap. Exemplary profile-based therapies include (a) effective administration of anti-inflammatory agents that are not immunosuppressive, e.g., some of the F1Cs and (b) effective administration of one or more antibiotics or growth or differentiation factors or other agents that enhance endogenous growth or differentiation of damaged or insufficient cells or tissues, e.g., 1, 2, or more of EPO, TPO, G-CSF, GM-CSF, IGF-1, α-1 thymosin, thymopoietin, serum thymic factor, biologically active fragments of any of these growth factors, polymer conjugates of any of these growth factors or their biologically active fragments or some of the steroids of formula 1. For some of these agents, the enhancement may be transient such as where a single administration or a pulse of synthesis occurs and the growth or differentiation factor is present in appreciable amounts for a limited time period, e.g. for a period of about 2-12 hours or for about 1, 2 or 3 days.

In other embodiments, the invention provides methods comprising, (1) providing or obtaining a subject who has been exposed to a biological insult that can potentially cause one or more potentially lethal biological responses; (2) measuring one, two or more of the subject's biological responses to the biological insult to obtain the subject's status profile with a defined Pₛᵤᵣᵥᵢᵥₐₗ or P_{lethality}; and (3) using the subject's status profile to identify or select one or more profile-based therapies.

In other embodiments, the invention provides methods comprising, (1) providing or obtaining a subject who has been exposed to a biological insult that will lead to a defined Pₛᵤᵣᵥᵢᵥₐₗ or P_{lethality}, optionally is at least about 0.9, at least about 0.95, at least about 0.98 or the P_{lethality} is at least about 0.1, at least about 0.05 or at least about 0.02 (2) measuring one, two or more of the subject's biological responses to the biological insult to obtain the subject's status profile with a defined Pₛᵤᵣᵥᵢᵥₐₗ or P_{lethality}; and (3) using the subject's status profile to identify or select one or more profile-based therapies. In these embodiments the subject's status profile may indicate that the subject has a probability of at least about 20%, at least about 30%, at least about 40%, at least about 50%, at least about 60% or at least about 70% of not surviving the exposure to the radiation dose without the use or application of one, two or more profile-based therapies.

As is apparent from the foregoing, the invention provides a method to obtain a status profile in an exposed subject comprising measuring temperature continuously or essentially continuously for sufficient time to detect fever or disruption of circadian rhythm or the initiation of fever or the existence fever, where the temperature measurements are at least partially obtained using an implanted or ingested temperature monitoring device. Implanted or ingested telemetric transmitters such as model TA10EA-F20 or model TA10TAD70 (Data Sciences, St. Paul, Minn.) or other devices described herein can be used to continuously or frequently monitor one or more biological parameters such as core temperature, peripheral temperature, heart rate, electroencephalogram or brain electrical activity, SaO₂ or blood pressure. Methods and means to monitor temperature, heart rate and other parameters using implanted devices in humans and other subjects have been described and can be employed for appropriate subjects in any invention method or embodiment disclosed herein. See, e.g., M. Akita et al., Exp. Anim. 53:212-127 2004, M. Mojarradi et al., IEEE Trans. Neural Syst. Rehabil. Eng. 11:38-42 2003, E. A. Johannessen et al., IEEE Trans. Biomed. Eng. 51:525-535 2004, L. R. Leon et al., Am. J. Physiol. Regul. Comp. Physiol. 286:R967-974 2004, N. G. IIback and T. Stalhandske J. Vet. Med. A Physiol. Pathol. Clin. Med. 50:479-483 2003, D. L. Clark et al., Can. J. Physiol. Pharmacol. 81:880-883 2003, A. J. Davidson et al., J. Biol. Rhythms 18:430-432, F. Genin and M. Perret, Comp. Biochem. Physiol. B Biochem. Mol. Biol. 136:71-812003, J. W. Boles et al., Vaccine 21:2791-2796 2003, and C. Nadziejko et al., Cardiovasc. Toxicology 2:237-244 2002.

These methods include measurements of one or more of the subject's biological responses to the radiation exposure are the subject's temperature and 1, 2, 3 or more of the subject's neutrophil count, red blood cell count, hematocrit, platelet count, bone marrow cellularity, reticulocyte count, bleeding, lethargy, pain, decreased food consumption, serum enzyme level. Exemplary biological responses or parameters that are measured include (i) temperature, e.g., for fever that is at least transient, (ii) circadian rhythm disruption, (iii) platelets, e.g., at their nadir after the biological insult, (iv) red cells or hematocrit, e.g., at the nadir after the biological insult, (v) neutrophils, e.g., at their nadir after the biological insult, or (v) combinations of two or three of these such as (i) and (ii), (i) and (iii), (i) and (iv), (i) and (v), (ii) and (iii), (ii) and (iv), (ii) and (v), (iii) and (iv), (iii) and (v), (iv) and (v), (i), (iii) and (iv), (ii), (iii) and (iv), (i), (iii) and (v), (ii), (iii) and (iv), (i), (iv) and (iv), (ii), (iii) and (iv) or (ii), (iii) and (v). In any of these methods, (i) 1, 2 or more of the subject's biological responses to the radiation exposure are measured on 1, 2, 3, 4 or more occasions or they are measured essentially continuously, optionally in real time and (ii) optionally wherein 1, 2 or more of the palliative therapies are the same as 1, 2 or more of the profile-based therapies or response therapies. Typically the palliative therapies are not the same as any of the profile-based therapies or response therapies. Any of the palliative therapies the profile-based therapies or response therapies is optionally administered before, during or after the exposure of the subject to the biological insult. Status profile based therapies or response therapies include effective administration of a hematopoiesis stimulator, an immune system stimulator, an antiinflammatory agent, an anti-apoptosis agent or management of the subject's temperature and any of these are optionally maintained until the subject has sufficiently recovered from the radiation exposure to have a probability of surviving the radiation exposure of at least about 60% or at least about 70% from the time the one or more response therapies is discontinued.

In some embodiments, the subject is a human having cancer or a human undergoing a bone marrow transplant protocol, optionally where the cancer is lung cancer, prostate cancer, breast cancer, colon cancer, skin cancer, a cancer of the central or peripheral nervous system, cervical cancer or another cancer or precancer described herein or in the cited references.

Invention embodiments include various uses for the methods disclosed herein and various materials that can be used in the practice of the invention methods. In some embodiments, the invention provides a kit for measuring temperature in a subject that has been exposed to a biological insult comprising (i) one, two or a plurality of temperature measuring devices and (ii) instructions that directs use of the temperature measuring devices so as to (a) detect a fever or elevated temperature of at least about 0.5° C. or at least about 0.1° C. above a baseline or normal human temperature, optionally (b) within a time period of about 15 minutes to about 24 hours after a biological insult as described in any claim or elsewhere herein and/or (c) optionally wherein when the fever or elevated temperature is at least about 0.8° C. or at least about 1.0° C above baseline or normal human temperature or where the fever elevated temperature remains elevated above baseline or normal human temperature at least about 0.8° C. or by at least about 1.0° C. for at least about 15 minutes to about 24 hours, the elevated temperature (1) corresponds with a P_{lethality} or probability that the subject will survive biological insult of less than about 0.1 or less than about 0.05, or (2) the biological insult has a probability that is greater than about 50%, about 60% or about 70% of causing a life-threatening adverse or toxic biological response.

In these kits, the temperature measuring devices optionally are single use devices, optionally wherein the devices monitor only peak temperature during the period in which the temperature measuring devices are in use, optionally wherein the peak temperature is indicated by a color change or by a highlighted numeric temperature value. Such kits would be used, e.g., in triage situations where limited medical services are available. Identification of a temperature spike that corresponds to, e.g., a P_{lethality} of 0.2, 0.1, 0.05 or less or to a significant chance, e.g., greater than about 50%, that the exposed subject would not survive a biological insult, would be useful in these situations to identify exposed subjects, potentially exposed subjects and/or exposed subjects that would probably require significant medical attention or intervention to survive.

On reading the present disclosure including the examples below, it will be apparent to one of ordinary skill in the art that the P_{lethality}, Pₛᵤᵣᵥᵢᵥₐₗ or other status profile measures described, herein can be used to identify exposed subjects or other subjects that have a high probability of not surviving, absent aggressive medical intervention or even in spite of aggressive medical intervention. It will also be apparent that the status profile can predict this situation well in advance of the time that the exposed subject may succumb to a biological insult. In some cases, the status profile allows prediction of this possibility shortly before this crisis period, e.g., about 0.5 days, about 1 day, about 2 days or about 3 days before the potentially lethal crisis. Other profiles such as the temperature spike or the circadian rhythm disruption that is detectable shortly after a potentially lethal radiation exposure allows prediction many days in advance of the crisis, e.g., about 4, 5, 6, 7, 8, 9 10, 11, 12, 13, 14, 15, 16, 17, 18, 19 or more day in advance. The status profiles described and means to measure them as described herein are thus very useful parameters to have, both for human clinical uses and for general biological or physiological studies.

Dosages of F1C and dosing protocols or methods. In any of the methods disclosed herein that incorporate a F1C, one can continuously or intermittently administer the F1C(s) to a subject or an exposed subject. Exemplary dosing protocols are found at, e.g., international publication No. WO 2004/019953 A1, WO 02/069977 A1 and/or U.S. Pat. No. 6,667,299 B1. In any of the continuous or in any step(s) in an intermittent dosing protocol, or in performing any of the methods described herein, the F1C(s) can be administered by one or more suitable routes, e.g., oral, buccal, sublingual, intramuscular (i.m.), subcutaneous (s.c.), intravenous (i.v.), intradermal, another parenteral route or by an aerosol. The effective daily dose in such methods will typically comprise about 0.05 mg/kg/day to about 200 mg/kg/day, or about 0.1 to about 100 mg/kg/day, including about 0.2 mg/kg/day, 0.5 mg/kg/day, about 1 mg/kg/day, about 2 mg/kg/day, about 4 mg/kg/day, about 6 mg/kg/day, about 10 mg/kg/day, about 20 mg/kg/day, about 40 mg/kg/day or about 100 mg/kg/day. Higher dosages, e.g., about 250 mg/kg/day, about 300 mg/kg/day or about 350 mg/kg/day can also be utilized, e.g., in veterinary applications. One can administer the F1C(s) orally using about 4 to about 60 mg/kg/day, usually about 6-30 mg/kg/day. In some embodiments, the intermittent dosing methods exclude dosing protocols that are commonly used to deliver contraceptive steroids to, e.g., human females, such as daily dosing for 21 days, followed by no dosing for 7 days. For humans, dosing is generally about 0.005 mg/kg/day to about 30 mg/kg/day, typically about 0.5-5 mg/kg/day. Low dosages for humans such as about 0.005 mg/kg/day to about 0.2 mg/kg/day or about 0.25-10 mg/day, can be used with, e.g., local, topical, transmucosal or intravenous administration and higher dosages such as about 0.1 mg/kg/day to about 20 mg/kg/day or about 5-200 mg/day, can be used, e.g., for oral, subcutaneous or other systemic or local administration route. For non-human subjects, e.g., mammals such as rodents or primates, the effective daily dosage may comprise about 0.05 mg/kg/day to about 350 mg/kg/day. F1C formulation dosages or daily doses or unit doses or subdoses for subjects such as humans and mammals include, e.g., about 1, 5, 10, 15, 20, 25, 50, 75, 100, 125, 150, 175, 200, 225, 250, 275, 300, 325, 350, 400 or 450 mg of the F1C.

Formulations and compositions for preparing formulations. Some invention methods include embodiments where formulations that contain an F1C are used. Such formulations have been described and may used in the present invention, see, e.g., international publication No. WO 2004/019953 A1, WO 02/069977 A1 and/or U.S. Pat. No. 6,667,299 B1. Formulations, e.g., one or more of oral, parenteral, topical, transmucosal, buccal, sublingual and/or aerosol formulations can be used.

Formula 1 compounds. Hematopoiesis stimulators or immune system stimulators include growth factors and hormones described herein, e.g., G-CSF, GM-CSF, IL-11 or a formula 1 compound (F1C) having the structure 5, 6, 7, 8, 9, 10, 11, 12, 13 or 14

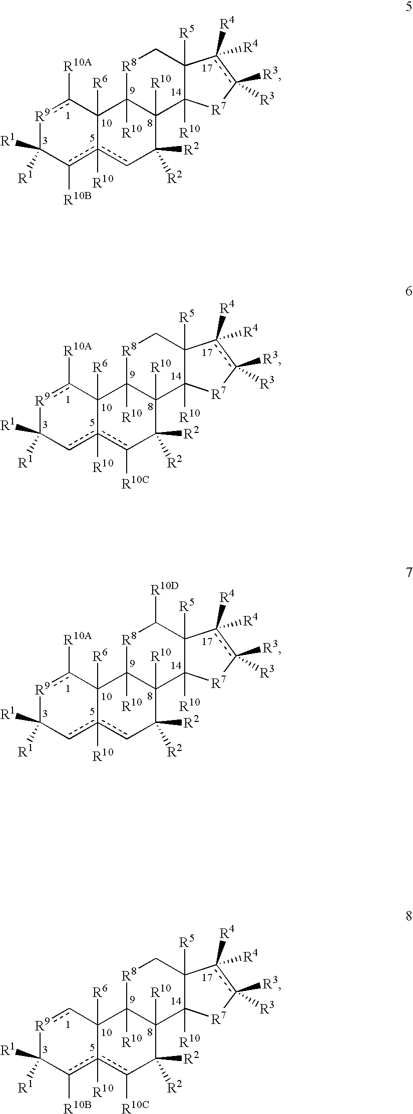

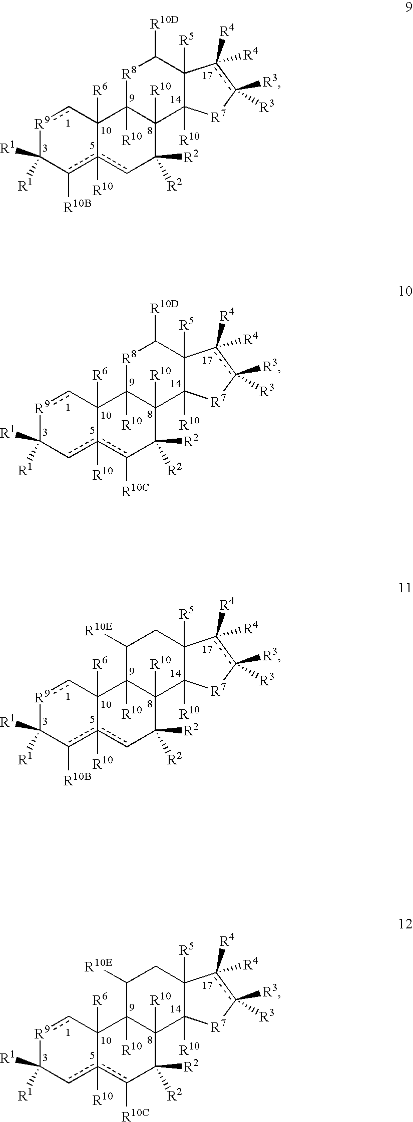

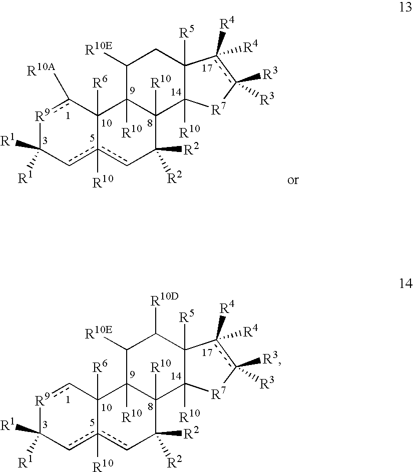

or a metabolic precursor or a metabolite thereof, wherein

R¹⁰ moieties at the 5 (if present), 8, 9 and 14 positions respectively are in the α,α,α,α, α,α,α, β, α,α,β,α, αβ,α,α, β,α,α,α, α,α,β,β, α,β,α, β, β,α,α,β, β,α,β,α, β,β,α,α, α,β,β,α, α,β,β,β, β,α,β,β, β,β,α,β, β,β, β,α or β,β,β,β configurations,

wherein R^{10A}, R^{10B}, R^{10C}, R^{10D} and R^{10E} respectively are in the α,α, α,β, β,α or β,β configurations,

wherein, each R¹, R², R³, R⁴, R⁵, R⁶, R¹⁰, R^{10A}, R^{10B}, R^{10C}, R^{10D} and R^{10E} independently are —H, —OH, —OR^{PR}, —SR^{PR}, —N(R^{PR})₂, —O—Si—(R¹³)₃, —CHO, —CHS, —CN, —SCN, —NO₂, —NH₂, —COOH, —OSO₃H, —OPO₃H, an ester, a thioester, a thionoester, a phosphoester, a phosphothioester, a phosphonoester, a phosphiniester, a sulfite ester, a sulfate ester, an amide, an amino acid, a peptide, an ether, a thioether, an acyl group, a thioacyl group, a carbonate, a carbamate, a halogen, an optionally substituted alkyl group, an optionally substituted alkenyl group, an optionally substituted alkynyl group, an optionally substituted aryl moiety, an optionally substituted heteroaryl moiety, an optionally substituted heterocycle, an optionally substituted monosaccharide, an optionally substituted oligosaccharide, a nucleoside, a nucleotide, an oligonucleotide, a polymer, or,

one more of R¹, R², R³, R⁴, R⁵, R⁵, R¹⁰, R^{10A}, R^{10B}, R^{10C}, R^{10D} and R^{10E} are ═O ═S, ═N—OH, ═CH₂, ═CH—CH₃, or an independently selected spiro ring and the hydrogen atom or the second variable group that is bonded to the same carbon atom is absent, or,

one or more of two adjacent R¹—R⁶, R¹⁰, R^{10A}, R^{10B}, R^{10C}, R^{10D} and R^{10E} comprise an independently selected epoxide, acetal, a thioacetal, ketal or thioketal;

R⁷ is —C(R¹⁰)₂—, —C(R¹⁰)₂—C(R¹⁰)₂—, —C( R¹⁰)₂—C(R¹⁰)₂—O—C(R¹⁰)₂—, —C(R¹⁰)₂—S—C(R¹⁰)₂—, —C(R¹⁰)₂—NR^{PR}—C(R¹⁰)₂—, —O—, —O—C(R¹⁰)₂—, —S—, —S—C(R¹⁰)₂—, —NR^{PR}— or —NR^{PR}—C(R¹⁰)₂—;

R⁸ and R⁹ independently are —C(R¹⁰)₂—, —C(R¹⁰)₂—C(R¹⁰)₂—, —O—, —O—C(R¹⁰)₂—, —S—, —S—C(R¹⁰)₂—, —NR^{PR}— or —NR^{PR}—C(R¹⁰)₂—, or one or both of R⁸ or R⁹ independently are absent, leaving a 5-membered ring;

R¹³ independently is C₁₋₆ alkyl; and

R^{PR} independently is —H or a protecting group, optionally provided that (1) one or two of R^{10A}, R^{10B}, R^{10C}, R^{10D} and R^{10E} are not hydrogen or (2) one R⁴ is —NH₂, an opotionally substituted amine, —N(R^{PR})², ═NOH, ═NO-optionally substituted alkyl, an amide or an N-linked amino acid. In these embodiments, the subject may have or be subject to developing the listed condition and the subject can be a human or a primate.

For these F1Cs, exemplary embodiments include structures where one each of R¹, R², R³ and R⁴ are —H, and, when no double bond links the second R¹, R², R³ and R⁴ to the ring to which it is bonded and no double bond is present at the 16-17 position, then the second R¹, R², R³ and R⁴ respectively are in the α,α,α,α, α,α,α,β, α,α, β,α, α,β,α,α,β,α,α,α,α,α,β,β,α,β,α,β,β,α,α,β,β,α,α,β,β,α, β,α,α,β,β,α,β,α,β,β, β,β,α,β,β,β,β,α or β,β,β,β configurations and the second R¹, R², R³ and R⁴ are optionally independently selected from —H, —F, —Cl, —Br, —I, —OH, —SH, —NH₂, —COOH, —CH₃, —C₂H₅, —C(CH₃)₃, —OCH₃, —OC₂H₅, —CF₃, —CH₂OH, —C(O)CH₃, —C(O)CH₂OH, —C(O)CH₂F, —C(O)CH₂Cl, —C(O)CH₂Br, —C(O)CH₂I, —C(O)CF₃, —C₂F₅, ═O, ═CH₂, ═CHCH₃, amino acid, carbamate, carbonate, optionally substituted C1-C20 alkyl, optionally substituted C1-C20 ether, optionally substituted C1-C20 ester, optionally substituted C1-C20 thioether, optionally substituted C1-C20 thioester, optionally substituted monosaccharide, optionally substituted disaccharide, optionally substituted oligosaccharide.

Either of these embodiments include compounds where (a) R^{10A} is bonded to the ring to which it is attached by a single bond and a double bond is present at (i) the 1-2 position, or (ii) the 1-2 and 16-17 positions; or (b) R^{10B} is bonded to the ring to which it is attached by a single bond and a double bond is present at the 4-5 position; or (c) R^{10C} is bonded to the ring to which it is attached by a single bond and a double bond is present at the 5-6 position; or (d) R^{10A} and R^{10B} are bonded to the rings to which they are attached by a single bond and a double bond is present at (i) the 1-2 and 4-5 positions, or (ii) the 1-2, 4-5 and 16-17 positions; (e) R^{10A} and R^{10C} are bonded to the rings to which they are attached by a single bond and a double bond is present at (i) the 1-2 and 5-6 positions, or (ii) the 1-2, 5-6 and 16-17 positions; or (f) no double bond is present or (g) the compounds have the structure

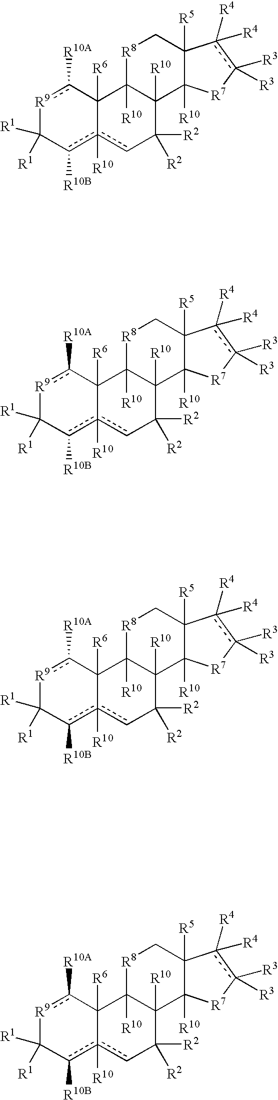

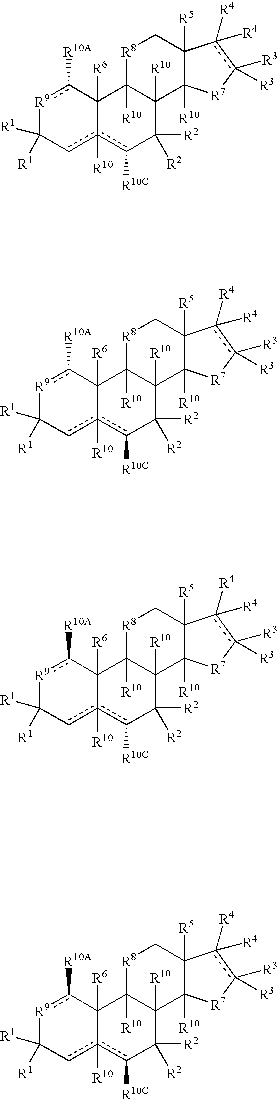

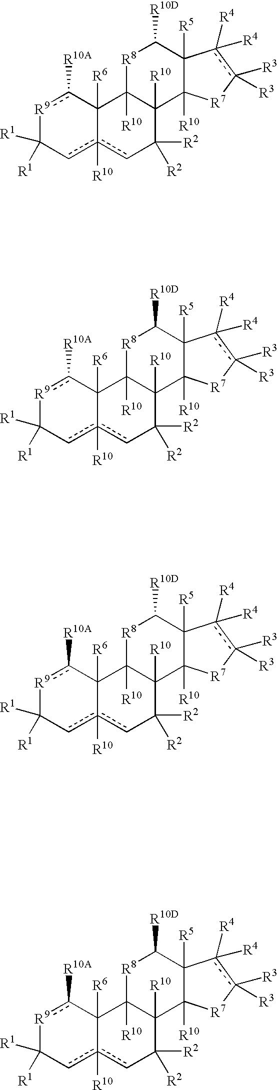

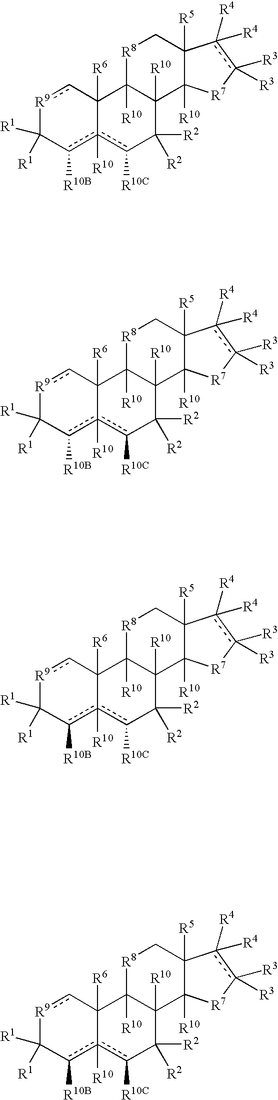

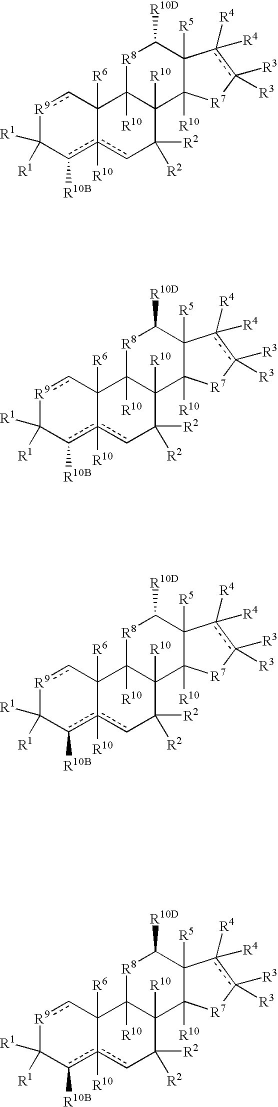

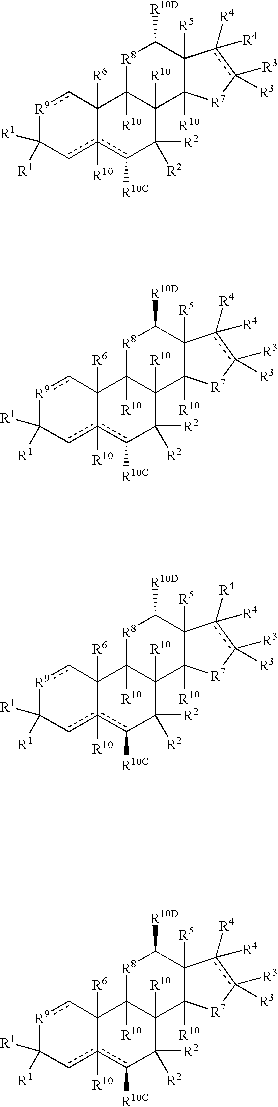

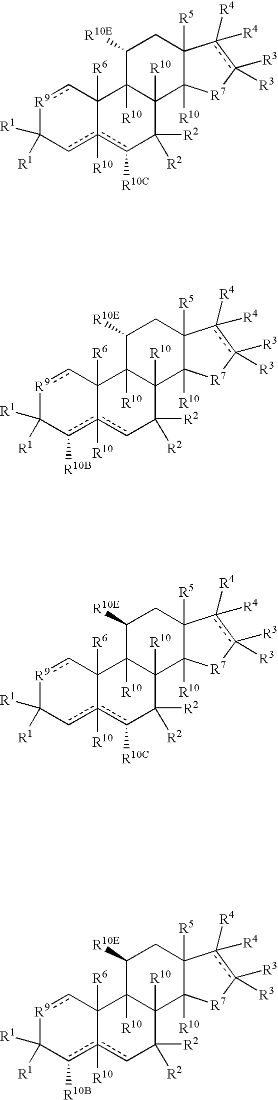

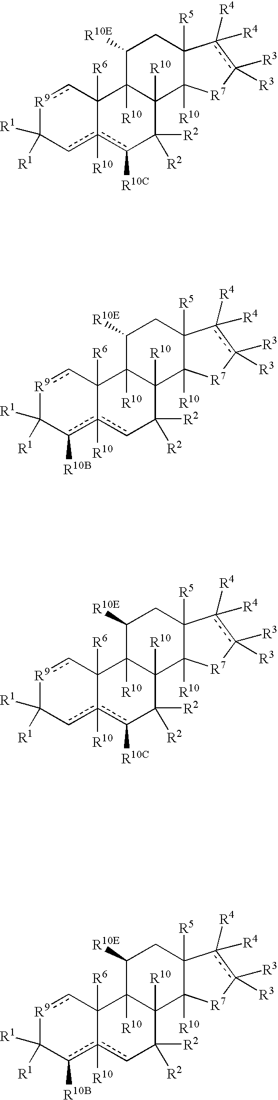

provided that if a double bond is present at the 1-2, 4-5 or 5-6 positions, then R^{10A}, R^{10B} or R^{10C} respectively are bonded to the ring to which they are linked by a single bond and wherein, when R¹, R², R³ and R⁴ are single bonded, one is in the α-configuration and the other R¹, R², R³ and R⁴ is in the β-configuration, optionally wherein (A) R⁵ and R⁶ respectively are in the α,α, α,β, β,α or β,β configuration and R⁵ and R⁶ are optionally both —CH₃ or are optionally selected from —CH₃ and —CH₂OH or (2) R⁵ and R⁶ are both in the β-configuration and R⁵ and R⁵ are optionally both —CH₃ or are optionally —CH₃ and —CH₂OH; and/or (B) R⁵ and R⁶ are optionally both in the β-configuration and are optionally independently selected from —H, —F, —Br, —CH₃, —C₂H₅, —C(CH₃)₃, —CH₂CH₂OH, —CH(O), —CH₂OH, —CH₂-ester, —CH₂-ether, —CH₂-amino acid, —CH₂-carbamate, —CH₂OR^{PR}, —CHS, —CH₂SH, —CH₂SR^{PR}, —CH₂-thioester, —CH₂-thioether, —CH₂NH₂, —CH₂NHR^{PR}, —CF₃, —CH₂CH₃, —CH₂CH₂F, —CH₂CF₃, —CH₂OC(O)—(CH₂)ₙ—CH₃, —CH₂OC(O)—(CH₂)ₙ—CO₂H, —CH₂OC(O)—(CH₂)n-CO₂R^{PR}, —CH₂OC(O)—(CH₂)ₙ—C(O)SH, —CH₂OC(O)—(CH₂)n-C(O)SR^{PR}, —CH₂OC(O)—(CH₂)ₙ—NH₂, —CH₂OC(O)—(CH₂)n-NHR^{PR}, a monosaccharide, and an ester wherein n is 0, 1, 2, 3 or 4 and R^{PR} are independently selected protecting groups for atoms to which they are bonded; and/or (C) one each of R¹, R², R³ and R⁴ are —H and wherein (i) no double bond is present at the 16-17 position, the second R¹, R², R³ and R⁴ respectively are bonded to the ring to which they are attached by a single bond in the β,β,α,β configurations (i.e., R¹ is in the β-configuration, R² is in the β-configuration, R³ is in the α-configuration and R⁴ is in the β-configuration when no double bond is present at 16-17), or (ii) a double bond is present at the 16-17 position and R¹ and R² respectively are in the β,β configurations (i.e., R¹ is in the β-configuration and R² is in the β-configuration when a double bond is present at 16-17); and/or (D) (i) no double bond is present at the 16-17 position, one each of R¹, R², R³ and R⁴ are —H, and the second R¹, R², R³ and R⁴ respectively are bonded to the ring to which they are attached by a single bond in the β,β,β,β configurations or (ii) one each of R¹, R² and R³ are —H, no double bond is present at the 16-17 position, the second R¹, R² and R³ respectively are bonded to the ring to which they are attached by a single bond in the β,β,β, β,β,α, β,α,β, α,β,β, β,α,α, α,β,α, α,α,β or α,α,α configurations and both R⁴ together are bonded to the ring by a double bond (i.e., both R⁴ together are a double bonded moiety described herein such as ═O, ═NOH, ═CH₂ or ═CH—CH₃); and/or (E) (i) no double bond is present at the 16-17 position, one each of R¹, R², R³ and R⁴ are —H and the second R¹, R², R³ and R⁴ respectively are bonded to the ring to which they are attached by a single bond in the β,β,β,α configurations or (ii) one each of R¹, R² and R⁴ are —H, no double bond is present at the 16-17 position, the second R¹, R² and R⁴ respectively are bonded to the ring to which they are attached by a single bond in the β,β,β, β,β,α, β,α,β, α,β,β, β,α,α, α,β, α, α,α,β or α,α,α configurations and both R³ together are bonded to the ring by a double bond (i.e., both R³ together are a double bonded moiety described herein such as ═O, ═NOH, ═CH₂ or ═CH—CH₃); and/or (F) no double bond is present at the 16-17 position, one each of R¹, R², R³ and R⁴ are —H and the second R¹, R², R³ and R⁴ respectively are bonded to the ring to which they are attached by a single bond in the β,β,α,α configurations or the α,β,β,β configurations; and/or (G) (i) no double bond is present at the 16-17 position, one each of R¹, R², R³ and R⁴ are —H, the second R¹, R², R³ and R⁴ respectively are bonded to the ring to which they are attached by a single bond in the β,α,β,β configurations (i.e., R¹ is in the β-configuration, R² is in the α-configuration, R³ is in the β-configuration and R⁴ is in the β-configuration when no double bond is present at 16-17), or (ii) a double bond is present at the 16-17 position and R¹ and R² respectively are in the β,α configurations (i.e., R¹ is in the β-configuration and R² is in the α-configuration when a double bond is present at 16-17) and/or (H) for any of these embodiments R¹⁰ at the 5 (if present), 8, 9 and 14 positions are in the α,β,α,α, β,β,α, αα,β,α,β or β,β,α,β configurations respectively; and/or (I) (i) no double bond is present at the 16-17 position, one each of R¹, R², R³ and R⁴ are —H, and the second R¹, R², R³ and R⁴ respectively are bonded to the ring to which they are attached by a single bond in the β,α,β,α configurations or (ii) one each of R¹, R³ and R⁴ are —H, no double bond is present at the 16-17 position, the second R¹, R³ and R⁴ respectively are bonded to the ring to which they are attached by a single bond in the β,β,β, β,β,α, β,α,β, α,β,β, β,α,α, α,β, α, α,α,β or α,α,α configurations and both R² together are bonded to the ring by a double bond (i.e., both R² together are a double bonded moiety described herein such as ═O, ═NOH, ═CH₂ or ═CH—CH₃).

As is apparent from the F1C structures, (i) when no double bond is present at the 4-5 or the 5-6 positions, R¹⁰ at the 5, 8, 9 and 14 positions respectively may be in the α,α,α,β, α,α,β,α, α,α,β,β, β,α,α,β, β,α,β,α, α,β,β,β, β,α, β,β, or β,β,β,β configurations or, (ii) if a double bond is present at the 4-5 or the 5-6 positions, then R¹⁰ at the 8, 9 and 14 positions respectively may be in the α,α,β, α,β,α, α,β,α or β,β,β configurations, and/or (iii) R¹⁰ at the 5 (if present), 8, 9 and 14-positions are independently selected from —H, —F, —Cl, —Br, —I, —OH, —OR^{PR}, —SH, —NH₂, —COOH, —CH₃, —C₂H₅, —C(CH₃)₃, —CH₂OH, —CH₂OR^{PR}, —CH₂F, —CH₂Cl, —CH₂Br, —CH₂I, —CHO, —CHS, —CH₂SH, —CH₂SR^{PR}, —CH₂NH₂, —CH₂NHR^{PR}, —CF₃, —CH₂CH₃, —CH₂CH₂F, —CH₂CF₃, —CH₂OC(O)—(CH₂)ₙ—CH₃, —CH₂OC(O)—(CH₂)ₙ—CO₂H, —CH₂OC(O)—(CH₂)n-CO₂R^{PR}, —CH₂OC(O)—(CH₂)ₙ—C(O)SH, —CH₂O)C(O)—(CH₂)ₙ—C(O)SR^{PR}, —CH₂OC(O)—(CH₂)ₙ—NH₂, —CH₂OC(O)—(CH₂)n-NHR^{PR}, a monosaccharide, an amino acid, a carbonate, a carbamate, an ester, optionally substituted C1-C20 alkyl optionally selected from —CH₃, —C₂H₅ and —C₃H₇, optionally substituted C1-C20 ether optionally selected from —OCH₃, —OC₂H₅ and —OC₃H₇, optionally substituted C1-C20 ester optionally selected from acetoxy and propionoxy, optionally substituted aryl optionally selected from —O-phenyl, —O-(alkoxy)₁₋₃-phenyl where each alkoxy is optionally independently selected (e.g., methoxy or ethoxy) and —O-(halo)₁₋₃-phenyl where each halogen is optionally independently selected (e.g., —F or —Cl), optionally where R¹⁰ at the 5, 8, 9 and 14-positions respectively are (1) —H, —H, —H, —H; (2) —H, —H, halogen (—F, —Cl, —Br or —I), —H; (3) —H, —H, —H, —OH; (4) —H, —H, halogen (—F, —Cl, —Br or —I), —OH; (5) -optionally substituted alkyl (e.g., —CH₃, —CH₂OH, —CH₂O-ester, —C₂H₅), —H, —H, —H; (6) -optionally substituted alkyl (e.g., —CH₃, —CH₂OH, —CH₂O-ester, —C₂H₅), —H, halogen (—F, —Cl, —Br or —I), —H; (7)-optionally substituted alkyl (e.g., —CH₃, —CH₂OH, —CH₂O-ester, —C₂H₅), —H, —H, —OH; (8)-acyl (e.g., —C(O)—(CH₂)₀₋₂—CH₃), —H, —H, —H; (9)-ester (e.g., acetoxy or propionoxy), —H, —H, —H; (10)-ether (e.g., —O—(CH₂)₀₋₂—CH₃), —H, —H, —H; (11)-ester (e.g., acetoxy, propionoxy, —O—C(O)—(CH₂)₁₋₆—H), —H, halogen (e.g., —F, —Cl, —Br), —H; (12)-ester (e.g., acetoxy or propionoxy), —H, —H, —OH; (13) —H, —H, —H, -acyl (e.g., —C(O)—(CH₂)₀₋₂—CH₃); (14) —H, —H, —H, -ester (e.g., acetoxy or propionoxy); or (15) —H, —H, —H, -ether (e.g., —O—(CH₂)₀₋₂—CH₃, —OCH₃, —OC₂H₅, —OCH₂OH, —OCH₂F, —OCH₂Br, —OCH₂COOH, —OCH₂NH₂, —OCH₂CH₂OH, —OCH₂CH₂F, —OCH₂CH₂Br, —OCH₂CH₂COOH or —OCH₂CH₂NH₂). When present, R¹⁰ at the 5-position and/or at the 14-position in the α-configuration or the β-configuration are optionally selected from —H, —F, —Cl, —Br, —I, —OH, —OR^{PR}, —CH₃, —C₂H₅, —C(CH₃)₃, —CH₂OH, —CH₂OR^{PR}, —CH₂F, —CH₂Cl, —CH₂Br, —CH₂I, —CH(O), —CH(S), —CH₂SH, —CH₂SR^{PR}, —CH₂NH₂, —CH₂NHR^{PR}, —CF₃, —CH₂CH₃, —CH₂CH₂F, —CH₂CF₃, —CH₂OC(O)—(CH₂)ₙ—CH₃, —CH₂OC(O)—(CH₂)ₙ—CO₂H, —CH₂OC(O)—(CH₂)n-CO₂R^{PR}, —CH₂OC(O)—(CH₂)ₙ—C(O)SH, —CH₂OC(O)—(CH₂)ₙ—C(O)SR^{PR}, —CH₂OC(O)—(CH₂)ₙ—NH₂, —CH₂OC(O)—(CH₂)ₙ—NHR^{PR}, a monosaccharide, an amino acid, a carbonate, a carbamate and an ester. Exemplary F1Cs include 3β-hydroxy-17β-aminoandrost-5-ene, 3β-amino-17β-hydroxy-17α-optionally substituted alkyl-androst-5-ene, 3β-hydroxy-9α-fluoro-17β-aminoandrost-5-ene, 3β-hydroxy-17β-amino-19-norandrost-5-ene, 3β,17β-dihydroxyandrost-5-ene, 3β,7β,17β-trihydroxyandrost-5-ene, 3β,7β,17β-trihydroxyandrost-1,5-diene, 3β,7β,17β-trihydroxy-19-norandrost-5-ene, 3β,17β-dihydroxy-19-norandrost-5-ene, 3β,17β-dihydroxy-17α-optionally substituted alkyl-19-norandrost-5-ene, 3β,17β-dihydroxy-3α,17α-dioptionally substituted alkyl-19-norandrost-5-ene or an 11-oxa, 2-oxa, or 9α-fluoro analog of any of these compounds.

In some embodiments one or more F1Cs or groups of F1Cs may be excluded from one or more of the uses disclosed herein. For example, if the subject has or is susceptible to developing a memory impairing neurological disorder or memory impairment condition, excluded compounds can include 5-androstene-3β-ol-7,17-dione or 5-androstene-3β,7-diol-17-one or a derivative of these compounds that can has a group at the 7-position that can convert to —OH or ═O by hydrolysis. In other cases, the F1Cs can exclude one or more of 4-pregnene-11β,17α,21-triol-3,20-dione, 17α,21-dihydroxypregn-4-ene-3,11,20-trione, 11β,21-dihydroxy-3,20-dioxopregn-4-en-18-al, 11β,17α,21-trihydroxypregna-1,4-diene-3,20-dione, 17α,21-dihydroxypregna-1,4-diene-3,11,20-trione, 3β-hydroxypregn-5-ene-20-one, 3β-hydroxyandrost-5-ene-17-one, pregn-4-ene-3,20-dione, 21-hydroxypregn-4-ene-3,20-dione, 9-fluoro-11β,16α,21-trihydroxy-16-methylpregna-1,4-diene-3,20-dione, 9-fluoro-11β,16α,17,21-tetrahydroxypregna-1,4-diene-3,20-dione, 9-fluoro-11β,17α,21-trihydroxy-16-methylpregna-1,4-diene-3,20-dione, dehydroepiandrosterone-3-sulfate, 1,4-pregnadiene-17α,21-diol-3,11,20-trione, androsterone, androsterone acetate, androsterone propionate, androsterone benzoate, androst-5-ene-3β,17β-diol, androst-5-ene-3β,17β-diol-3-acetate, androst-5-ene-3β,17β-diol-17-acetate, androst-5-ene-3β,17β-diol-3,17-diacetate, androst-5-ene-3β,17β-diol-17-benzoate, androst-5-ene-3β,17β-diol-3-acetate-17-benzoate, androst-4-ene-3,17-dione, androst-5-ene-3β,7β,17β-triol, androst-5-ene-3β,7α,17β-triol, dehydroepiandrosterone, 4-dihydrotestosterone, 5α-dihydrotestosterone, dromostanolone, dromostanolone propionate, ethylestrenol, nandrolone phenpropionate, nandrolone decanoate, nandrolone furylpropionate, nandrolone cyclohexanepropionate, nandrolone benzoate, nandrolone cyclohexanecarboxylate, oxandrolone, stanozolol, testosterone, methyl testosterone, testolactone, oxymetholone, fluoxymesterone, acetoxypregnenolone, allylestrenol, anagestone acetate, chlormadinone acetate, cyproterone, cyproterone acetate, desogestrel, dihydrogesterone, dimethisterone, ethisterone (17α-ethynyltestosterone), ethynodiol diacetate, flurogestone acetate, gestadene, hydroxyprogesterone, hydroxyprogesterone acetate, hydroxyprogesterone caproate, 3-ketodesogestrel, hydroxymethylprogesterone, hydroxymethylprogesterone acetate, levonorgestrel, lynestrenol, medrogestone, medroxyprogesterone acetate, megestrol, megestrol acetate, melengestrol acetate, norethindrone, norethindrone acetate, norethisterone, norethisterone acetate, norethynodrel, norgestimate, norgestrel, norgestrienone, normethisterone, and progesterone, progesterone, cyproterone acetate, norethindrone, norethindrone acetate, levonorgestrel, an ester of any of the foregoing compounds (e.g., acetate, enanthate, propionate, isopropionate, cyclopropionate, isobutyrate, butyrate, valerate, caproate, isocaproate, hexanoate, heptanoate, octanoate, nonanoate, decanoate, undecanoate, phenylacetate or benzoate esters, e.g., hydroxyl esters), a naturally occurring glucorcorticoid, a species disclosed herein or a derivative of any of these that can convert to these molecules by hydrolysis or metabolism, e.g., a metabolizable or hydrolyzable ester or ether such as a cyclic ketal, an acetate, a diacetete, a proprionate, a diproprionate, or an an O-alkyl, an acyl, e.g., —C(O)—C1-C6 alkyl or another moiety that is bonded at, e.g., a variable group such as for R¹—R⁶.

Numbered embodiments. The following numbered embodiments illustrate aspects of the invention or related subject matter.

1. A method to determine a status profile for a subject or a subject species comprising, (1) exposing a subject(s) to a biological insult of at least about an LD_{10/60} to obtain exposed treated subjects; (2) measuring on two or more occasions in or from the exposed subjects one, two or more biological parameters selected from temperature, circadian rhythm, red blood cell counts, hematocrit, reticulocytes, platelets, megakaryocytes and neutrophils; (3) measuring the survival rate of the exposed subjects; (4) obtaining one or more status profiles that corresponds to a defined probability of surviving the biological insult (Pₛᵤᵣᵥᵢᵥₐₗ) of at least 0.95 or of not surviving the biological insult (P_{lethality}) of at least 0.05; and (5) optionally using the status profile to identify and initiate a profile-based therapy for one or more of the exposed subjects or for other subjects of the same species or a closely related species that has been or that may have been exposed to a biological insult that is the same as or similar to that of step (1).

2. The method of embodiment 1 wherein the biological insult is about an LD_{30/60} to about an LD_{70/60} or wherein the biological insult is about an LD_{50/60}.

3. The method of embodiment 1 or 2 wherein the temperature is core body temperature, which is optionally measured (i) using an implanted monitor, and/or (ii) continuously and/or (iii) at intervals of about 1 minute, about 5 minutes or about 10 minutes to about 30 minutes, about 1 hour or about 2 hours.

4. The method of embodiment 1, 2 or 3 wherein the biological insult is a radiation exposure, optionally selected from γ-radiation, X-rays, β-radiation, fast neutrons and slow neutrons.

5. The method of embodiment 1, 2, 3 or 4 further comprising measuring on one or more occasions in or from the exposed subjects one or more biological parameters selected from macrophages, monocytes or a monocyte precursor, C reactive protein, fibrinogen, sepsis, respiration rate, pulse rate, blood or arterial pH, blood pressure, pH or composition of sweat, pH or composition of saliva, respired breath composition, urine pH or composition, blood SaO₂ or oxygen saturation of arterial oxyhemoglobin (e.g., as measured by a pulse oximeter), optionally selected from one, two or more of rapid eye movement sleep, sleeping brain theta waves, leptin, glucose, insulin, melatonin, heart rate, temperature, locomotor activity, autonomic nervous function, hormone, glucocorticoid levels such as cortisol levels, blood enzyme levels, B-cells, T-cells, natural killer cells, dendritic cells, neutrophils, eosinophils, basophils, CFU-Eos, CFU-Baso, neutrophil a neutrophil precursor, myeloblasts, complement protein C3a, sepsis, septic shock, myelocytes and neurological damage.

6. The method of embodiment 1, 2, 3, 4 or 5 wherein the subject is a non-human primate.

7. The method of embodiment 1, 2, 3, 4, 5 or 6 further comprising administering one or more palliative therapies to treat one or more side effects of the biological insult to obtain treated subjects and comparing the survival rate of the exposed subjects with the exposed treated subject(s).

8. The method of embodiment 1, 2, 3, 4, 5, 6, 7 or 8 wherein the status profile is based on measurements of one or two of elevated temperature, circadian rhythm disruption, platelet decrease, neutrophil decrease, hemoglobin decrease, red blood cell decrease and hematocrit decrease.

9. The method of embodiment 1, 2, 3, 4, 5, 6, 7 or 8 wherein the status profile is based on (i) a temperature increase of at least about 0.8° C. above baseline or at least about 1.0° C. above baseline or at least about 1.2° C. above baseline for a period of at least about 1 hour and a decrease of at least about 80% in red blood cell counts, hematocrit and/or reticulocytes; (ii) a temperature increase of at least about 1.0° C. above baseline for a period of at least about 30 minutes and a decrease of at least about 80% in red blood cell counts, hematocrit and/or reticulocytes at one or more time points; (iii) a temperature increase of at least about 1.5° C. above baseline for a period of at least about 15 minutes and a decrease of at least about 80% in red blood cell counts, hematocrit and/or reticulocytes at one or more time points; (iv) a temperature increase of at least about 3° C. above baseline for a period of at least about 3 hours and a decrease of at least about 15% in red blood cell counts, hematocrit and/or reticulocytes at one or more time points; and/or (v) another parameter or clinical condition or situation described herein.

10. The method of embodiment 1, 2, 3, 4, 5, 6, 7, 8 or 9 wherein one or more status profiles are obtained by unpaired t-test analysis, paired t-test analysis or another method described herein.

11. The method of embodiment 1, 2, 3, 4, 5, 6, 7, 8, 9 or 10 wherein the profile-based therapy is effective administration of an apoptosis inhibitor, α-1 thymosin, a hematopoiesis stimulator or an immune system stimulator optionally selected from one or more of G-CSF, GM-CSF, TPO, EPO, stem cell factor, pegfilgrastim and formula 1 compound, wherein the formula 1 compound optionally is 3β-hydroxy-17β-aminoandrost-5-ene, 3β-hydroxy-17β-aminoandrost-4-ene, 3β-hydroxy-17β-aminoandrost-1-ene, 3α-hydroxy-17β-aminoandrost-5-ene, 3α-hydroxy-17β-aminoandrost-4-ene, 3α-hydroxy-17β-aminoandrost-1-ene, 3β-hydroxy-17β-aminoandrost-1,5-diene, 3β-hydroxy-17β-aminoandrost-1,4-diene or a 19-nor, 2-oxa, 11-oxa, 2α-hydroxy, 2β-hydroxy, 2α-alkoxy, 2β-alkoxy, 7β-hydroxy, 7β-optionally substituted alkyl, 11-optionally substituted alkyl, 3α-optionally substituted alkyl and/or 3β-optionally substituted alkyl analog of any of these compounds, wherein the optionally substituted alkyl groups independently optionally contain 1, 2, 3, 4, 5 or 6 carbon atoms and/or 1 or 2 double bonds and/or triple bonds or wherein the formula 1 compound is any formula 1 compound described in any embodiment, compound group or chemical structure described anywhere herein.

12. The method of embodiment 1, 2, 3, 4, 5, 6, 7, 8, 9, 10 or 11 wherein the biological insult is a radiation dose of about 2 Gy to about 10 Gy of ionizing radiation, or wherein the radiation dose is about 6 Gy.

13. A method comprising, (1) providing a subject who has been exposed to a biological insult optionally selected from a radiation dose of at least about an LD₅; (2) measuring one, two or more of the subject's biological parameters or biological responses to the radiation exposure to obtain the subject's status profile, wherein the subject's status profile indicates (i) that the subject has a probability of at least about 50% of not surviving the exposure to the radiation dose or (ii) the subject will not survive the biological insult with a P_{lethality} of 0.10, 0.05 or less; (3) using the subject's status profile to identify one or more profile-based therapies; (4) optionally administering one or more palliative therapies to the subject; (5) optionally initiating the one or more profile-based therapies; and (6) optionally maintaining at least one of the one or more response therapies until the subject has sufficiently recovered from the radiation exposure to have a probability of surviving the radiation exposure of at least about 60% from the time the one or more response therapies is discontinued.

14. The method of embodiment 13 wherein the subject's status profile indicates that the subject has a probability of at least about 60% or at least about 70% of not surviving the exposure to the radiation dose, or wherein the P_{lethality} is at least about 0.6 or 0.7.

15. The method of embodiment 13 or 14 wherein the radiation dose is about an LD₁₀, about an LD₂₀, about an LD₃₀, about an LD₄₀, about an LD₅₀, about an LD₆₀, about an LD₇₀, about an LD₈₀, about an LD₉₀ or about an LD₁₀₀, where survival is measured at 30 days or at 60 days, or wherein the radiation dose is about 2 Gy to about 10 Gy, or wherein the radiation dose is about 6 Gy.

16. The method of embodiment 13, 14 or 15 wherein the two or more of the subject's biological parameters or biological responses to the radiation exposure are (i) temperature, circadian rhythm, red blood cell counts, hematocrit, reticulocytes, platelets, megakaryocytes or neutrophils; and optionally (ii) one or more biological parameters selected from macrophages, monocytes or a monocyte precursor, C reactive protein, fibrinogen, sepsis, respiration rate, pulse rate, blood or arterial pH, blood pressure, pH or composition of sweat, pH or composition of saliva, respired breath composition, urine pH or composition, blood SaO₂ or oxygen saturation of arterial oxyhemoglobin (e.g., as measured by a pulse oximeter), optionally selected from one, two or more of rapid eye movement sleep, sleeping brain theta waves, leptin, glucose, insulin, melatonin, heart rate, temperature, locomotor activity, autonomic nervous function, hormone, glucocorticoid such as cortisol, blood enzyme levels, B-cells, T-cells, natural killer cells, dendritic cells, neutrophils, eosinophils, basophils, CFU-Eos, CFU-Baso, neutrophil a neutrophil precursor, myeloblasts, complement protein C3a, sepsis, septic shock, myelocytes and neurological damage.

17. The method of embodiment 13, 14, 15 or 16 wherein temperature is the only biological parameter that is measured and the temperature is core body temperature, which is optionally measured (i) using an implanted monitor, and/or (ii) continuously and/or (iii) at intervals of about 1 minute, about 5 minutes or about 10 minutes to about 20 minutes, about 30 minutes, about 1 hour or about 2 hours.

18. The method of embodiment 13, 14, 15, 16 or 17 wherein the disruption of circadian rhythm the only biological parameter that is measured.

19. The method of embodiment 13, 14, 15, 16, 17 or 18 wherein the biological insult is a radiation exposure, optionally selected from γ-radiation, X-rays, β-radiation, fast neutrons and slow neutrons, optionally selected from the group consisting of ⁶⁰Co and ¹²⁷Cs radiation, and optionally wherein the dose rate is about 50 cGy/minute, about 60 cGy/minute or about 70 cGy/minute.

20. The method of embodiment 13, 14, 15, 16, 17, 18 or 19 wherein a profile-based therapy is administered and the profile-based therapy is effective administration of an apoptosis inhibitor, α-1 thymosin, a hematopoiesis stimulator, an immune system stimulator optionally selected from one or more of G-CSF, GM-CSF, TPO, EPO, stem cell factor, pegfilgrastim and formula 1 compound, wherein the formula 1 compound optionally is 3β-hydroxy-17β-aminoandrost-5-ene, 3β-hydroxy-17β-aminoandrost-4-ene, 3β-hydroxy-17β-aminoandrost-1-ene, 3α-hydroxy-17β-aminoandrost-5-ene, 3α-hydroxy-17β-aminoandrost-4-ene, 3α-hydroxy-17β-aminoandrost-1-ene, 3β-hydroxy-17β-aminoandrost-1,5-diene, 3β-hydroxy-17β-aminoandrost-1,4-diene or a 19-nor, 2-oxa, 11-oxa, 2α-hydroxy, 2β-hydroxy, 2α-alkoxy, 2β-alkoxy, 7β-hydroxy, 7β-optionally substituted alkyl, 11α-optionally substituted alkyl, 11β-optionally substituted alkyl, 17β-optionally substituted alkylamino (where —NH-optionally substituted alkyl is present at the 17-position), 17β-di(optionally substituted alkylamino) (where —NH-(optionally substituted alkyl)₂ is present at the 17-position), 3α-optionally substituted alkyl and/or 3β-optionally substituted alkyl analog of any of these compounds, wherein the optionally substituted alkyl groups independently optionally contain 1, 2, 3, 4, 5 or 6 carbon atoms and/or 1 or 2 double bonds and/or triple bonds and wherein all optionally substituted alkyl groups are independently chosen.

21. The method of embodiment 20 wherein the formula 1 compound contains 1, 2, 3, 4 or 5 ring double bonds and has the structure 5, 6, 7, 8, 9, 10, 11, 12, 13 or 14

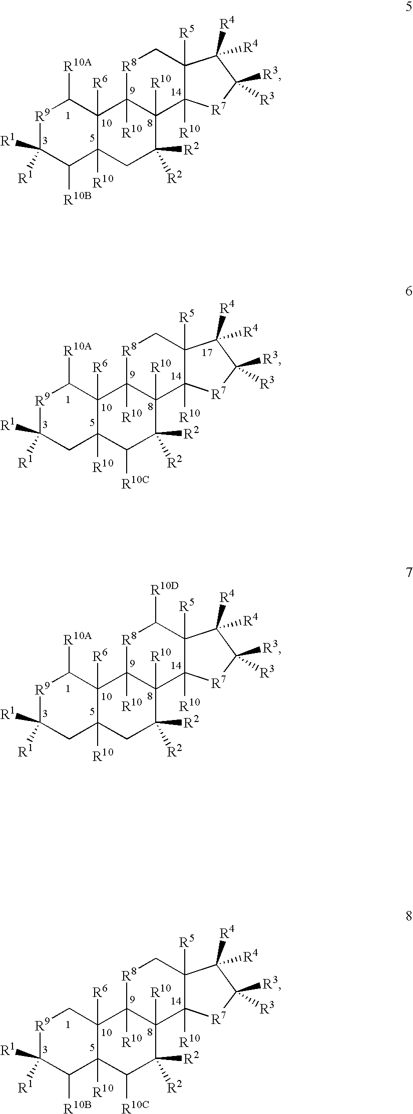

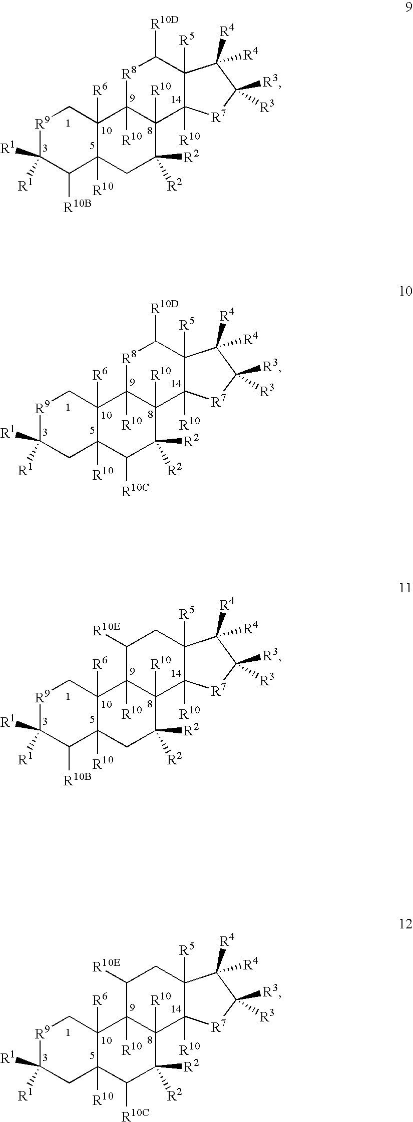

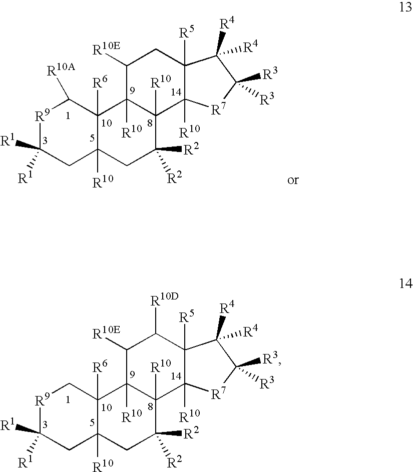

or a metabolic precursor or a metabolite thereof, wherein R¹⁰ moieties at the 5 (if present), 8, 9 and 14 positions respectively are in the α,α,α,α, α,α,α,β, α,α,β,α, α,β,α,α, β,α,α,α, α,α,β,β, α,β,α,β, β,α,α,β, β,α,β,α, β,β,α, α, α,β,β,α, α,β,β,β, β,α,β,β, β,β,α,β, β,β,β, α or β,β,β,β configurations and wherein R^{10A}, R^{10B}, R^{10C}, R^{10D} and R^{10E} respectively are in the α,α,α,β,β,α or β,β configurations; each R¹, R², R³, R⁴, R⁵, R⁶, R¹⁰, R^{10A}, R^{10B}, R^{10C}, R^{10D} and R^{10E} independently are —H, —OH, —OR^{PR}, —SR^{PR}, —N(R^{PR})₂, —O—Si—(R¹³)₃, —CHO, —CHS, —CN, —SCN, —NO₂, —NH₂, —COOH, —OSO₃H, —OPO₃H, an ester, a thioester, a thionoester, a phosphoester, a phosphothioester, a phosphonoester, a phosphiniester, a sulfite ester, a sulfate ester, an amide, an amino acid, a peptide, an ether, a thioether, an acyl group, a thioacyl group, a carbonate, a carbamate, a halogen, an optionally substituted alkyl group, an optionally substituted alkenyl group, an optionally substituted alkynyl group, an optionally substituted aryl moiety, an optionally substituted heteroaryl moiety, an optionally substituted heterocycle, an optionally substituted monosaccharide, an optionally substituted oligosaccharide, a nucleoside, a nucleotide, an oligonucleotide, a polymer, or, one more of R¹, R², R³, R⁴, R⁵, R⁶, R¹⁰, R^{10A}, R^{10B}, R^{10C}, R^{10D} and R^{10E} are ═O, ═S, ═N—OH, ═CH₂, ═CH—CH₃, or an independently selected spiro ring and the hydrogen atom or the second variable group that is bonded to the same carbon atom is absent, or, one or more of two adjacent R¹—R⁶, R¹⁰, R^{10A}, R^{10B}, R^{10C}, R^{10D} and R^{10E} comprise an independently selected epoxide, acetal, a thioacetal, ketal or thioketal; R⁷ is —C(R¹⁰)₂—, —C(R¹⁰)₂—C(R¹⁰)₂—, —C(R¹⁰)₂—C(R¹⁰)₂—C(R¹⁰)₂—, —C(R¹⁰)₂—O—C(R¹⁰)₂—, —C(R¹⁰)₂—S—C(R¹⁰)₂—, —C(R¹⁰)₂—NR^{PR}—C(R¹⁰)₂—, —O—, —O—C(R¹⁰)₂—, —S—, —S—C(R¹⁰)₂—, —NR^{PR}— or —NR^{PR}—C(R¹⁰)₂—; R⁸ and R⁹ independently are —C(R¹⁰)₂—, —C(R¹⁰)₂—C(R¹⁰)₂—, —O—, —O—C(R¹⁰)₂—, —S—, —S—C(R¹⁰)₂—, —NR^{PR}— or —NR^{PR}—C(R¹⁰)₂—, or one or both of R⁸ or R⁹ independently are absent, leaving a 5-membered ring; R¹³ independently is C₁₋₆ alkyl; and R^{PR} independently or together are —H or a protecting group.

22. The method of embodiment 21 wherein one each of R¹, R², R³ and R⁴ are —H, and, when no double bond links the second R¹, R², R³ and R⁴ to the ring to which it is bonded and no double bond is present at the 16-17 position, then the second R¹, R², R³ and R⁴ respectively are in the α,α,α,α, α,α,α,β, α,α,β,α, α,β,α,α, β,β,α,α, α,α,β,β, α,β,α,β,β,α,α,β,β,α,β,α, β,β,α,α, α,β,β,α, α,β,β,β, β,α,β, β,β,β,α,β,β,β,β,α or β,β,β,β configurations and the second R¹, R², R³ and R⁴ are optionally independently selected from —H, —F, —Cl, —Br, —I, —OH, —SH, —NH₂, —COOH, —CH₃, —C₂H₅, —C(CH₃)₃, —OCH₃, —OC₂H₅, —CF₃, —CH₂OH, —C(O)CH₃, —C(O)CH₂OH, —C(O)CH₂F, —C(O)CH₂Cl, —C(O)CH₂Br, —C(O)CH₂I, —C(O)CF₃, —C₂F₅, ═O, ═CH₂, ═CHCH₃, amino acid, carbamate, carbonate, optionally substituted C1-C20 alkyl, optionally substituted C1-C20 ether, optionally substituted C1-C20 ester, optionally substituted C1-C20 thioether, optionally substituted C1-C20 thioester, optionally substituted monosaccharide, optionally substituted disaccharide, optionally substituted oligosaccharide.

23. The method of embodiment 21 or 22 wherein (a) R^{10A} is bonded to the ring to which it is attached by a single bond and a double bond is present at (i) the 1-2 position, or (ii) the 1-2 and 16-17 positions; or (b) R^{10B} is bonded to the ring to which it is attached by a single bond and a double bond is present at the 4-5 position; or (c) R^{10c} is bonded to the ring to which it is attached by a single bond and a double bond is present at the 5-6 position; or (d) R^{10A} and R^{10B} are bonded to the rings to which they are attached by a single bond and a double bond is present at (i) the 1-2 and 4-5 positions, or (ii) the 1-2, 4-5 and 16-17 positions; (e) R^{10A} and R^{10C} are bonded to the rings to which they are attached by a single bond and a double bond is present at (i) the 1-2 and 5-6 positions, or (ii) the 1-2, 5-6 and 16-17 positions; or (f) no double bond is present.

24. The method of embodiment 21, 22 or 23 wherein the compounds have the structure

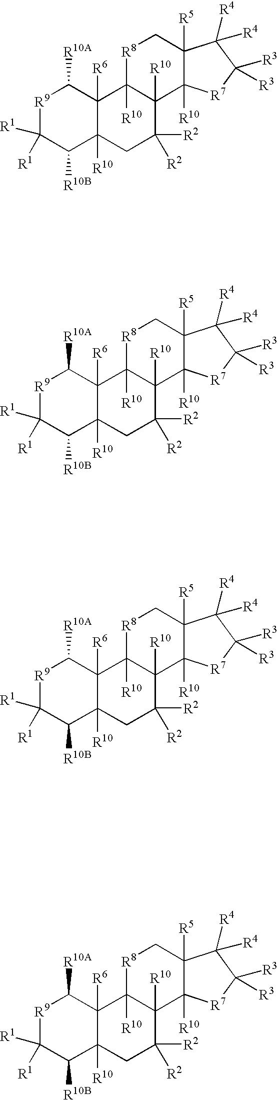

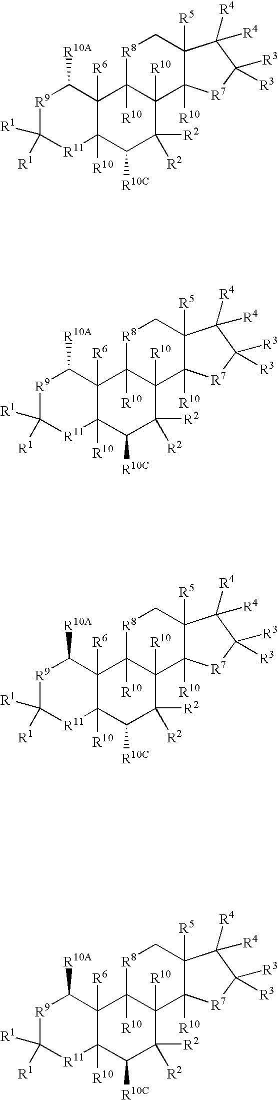

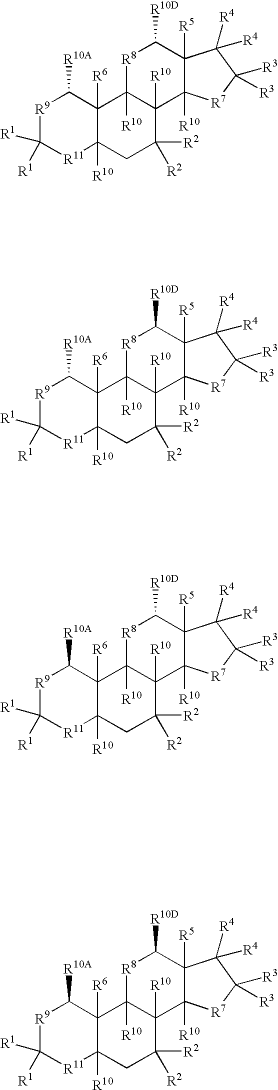

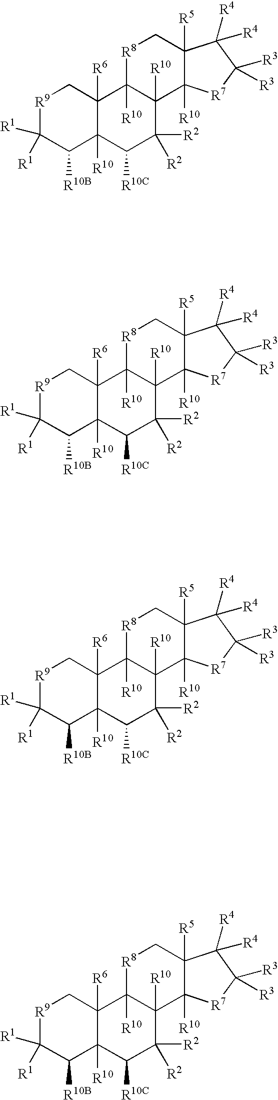

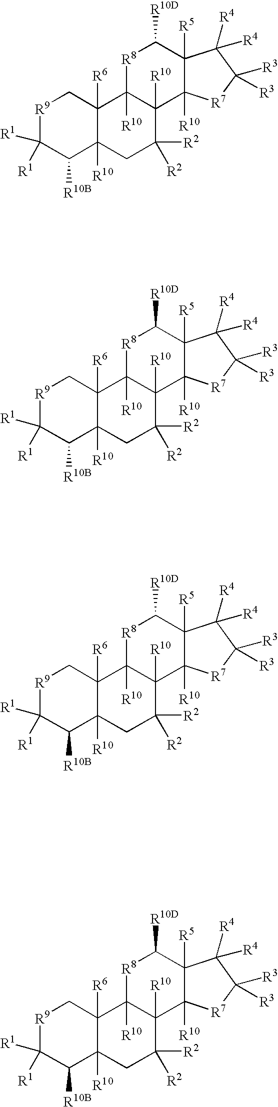

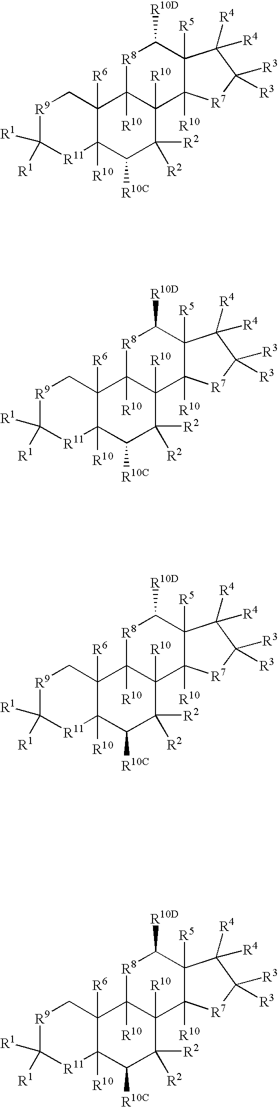

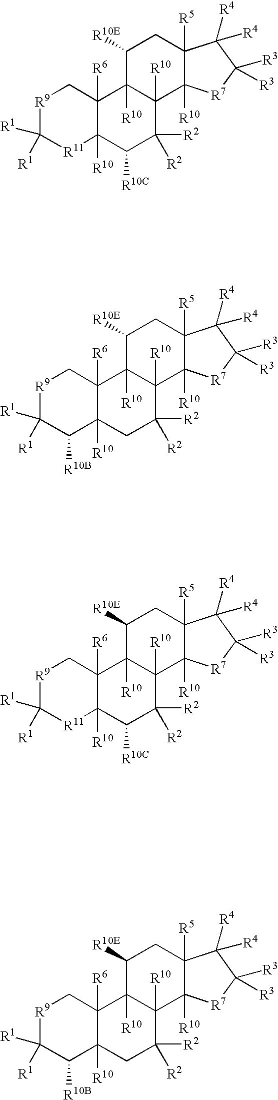

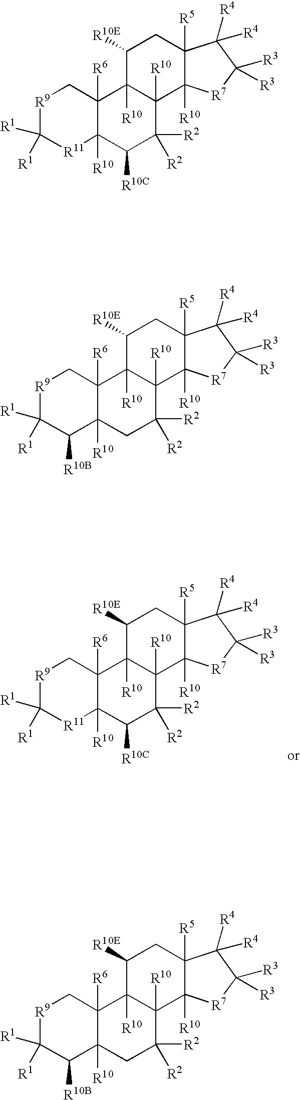

provided that if a double bond is present at the 1-2, 4-5 or 5-6 positions, then R^{10A}, R^{10B} or R^{10C} respectively are bonded to the ring to which they are linked by a single bond.

25. The method of embodiment 21, 22, 23 or 24 wherein (1) R⁵ and R⁵ respectively are in the α,α, α,β, β,α or β,β configuration and R⁵ and R⁵ are optionally both —CH₃ or are optionally selected from —H, —CH₃ and —CH₂OH or (2) R⁵ and R⁶ are both in the β-configuration and R⁵ and R⁶ are optionally both —H, —CH₃ or —CH₂OH.

26. The method of embodiment 21, 22, 23, 24 or 25 wherein R¹⁰ at the 5, 8, 9 and 14-positions respectively are (1) —H, —H, —H, —H; (2) —H, —H, halogen (—F, —Cl, —Br or —I), —H; (3) —H, —H, —H, —OH; (4) —H, —H, halogen (—F, —Cl, —Br or —I), —OH; (5) -optionally substituted alkyl (e.g., —CH₃, —CH₂OH, —CH₂O-ester, —C₂H₅), —H, —H, —H; (6) -optionally substituted alkyl (e.g., —CH₃, —CH₂OH, —CH₂O-ester, —C₂H₅), —H, halogen (—F, —Cl, —Br or —I), —H; (7) -optionally substituted alkyl (e.g., —CH₃, —CH₂OH, —CH₂O-ester, —C₂H₅), —H, —H, —OH; (8) -acyl (e.g., —C(O)—(CH₂)₀₋₂—CH₃), —H, —H, —H; (9) -ester (e.g., acetoxy or propionoxy), —H, —H, —H; (10) -ether (e.g., —O—(CH₂)₀₋₂—CH₃), —H, —H, —H; (11) -ester (e.g., acetoxy, propionoxy, —O—C(O)—(CH₂)₁₋₆—H), —H, halogen (e.g., —F, —Cl, —Br), —H; (12) -ester (e.g., acetoxy or propionoxy), —H, —H, —OH; (13) —H, —H, —H, -acyl (e.g., —C(O)—(CH₂)₀₋₂—CH₃); (14) —H, —H, —H, -ester (e.g., acetoxy or propionoxy); or (15) —H, —H, —H, -ether (e.g., —O—(CH₂)₀₋₂—CH₃, —OCH₃, —OC₂H₅, —OCH₂OH, —OCH₂F, —OCH₂Br, —OCH₂COOH, —OCH₂NH₂, —OCH₂CH₂OH, —OCH₂CH₂F, —OCH₂CH₂Br, —OCH₂CH₂COOH or —OCH₂CH₂NH₂).

27. The method of embodiment 21, 22, 23, 24, 25 or 26 wherein R⁷ is —O—, —S—, —NH—, —N(C1-C8 optionally substituted alkyl)-, —CH₂—, —CH(α-OH)—, —CH(β-OH)—, —C(O)—, —CH(α-SH)—, —CH(β-SH)—, —C(S)—, —CH(α-R¹⁰)—, —CH(β-R¹⁰)—, —CH(α-ester)-, —CH(β-ester)-, —CH(α-alkoxy)-, —CH(β-alkoxy)-, —CH(α-thioester)-, —CH(β-thioester)-, —CH(α-thioether)-, —CH(β-thioether)-, —CH(α-halogen)- or —CH(β-halogen)- where alkoxy group is an ether optionally selected from —OCH₃, —OC₂H₅ and —OC₃H₇ and the halogen atom is —F, —Cl, —Br or —I.

28. The method of embodiment 21, 22, 23, 24, 25, 26 or 27 wherein R⁸ is —CH₂—, —CF₂—, —O—, —S—, —NH—, —N(C1-C8 optionally substituted alkyl)-, —CH(α-OH)—, —CH(β-OH)—, —C(O)—, —CH(α-SH)—, —CH(β-SH)—, —C(S)—, —CH(α-R¹⁰)—, —CH(β-R¹⁰)—, —CH(α-ester)-, —CH(β-ester)-, —CH(α-alkoxy)-, —CH(β-alkoxy)-, —CH(α-thioester)-, —CH(β-thioester)-, —CH(α-thioether)-, —CH(β-thioether)-, —CH(α-halogen)- or —CH(β-halogen)- where alkoxy group is an ether optionally selected from —OCH₃, —OC₂H₅ and —OC₃H₇ and the halogen atom is —F, —Cl, —Br or —I.

29. The method of embodiment 21 wherein the formula 1 compound is 16α-bromo-3β-hydroxy-5α-androstan-17-one, 16α-bromo-3β-hydroxy-5α-androstan-17-one hemihydrate, 16α-fluoro-3β-hydroxy-5α-androstan-17-one, 16α-chloro-3β-hydroxy-5α-androstan-17-one, 16β-bromo-3β-hydroxy-5α-androstan-17-one, 16β-fluoro-3β-hydroxy-5α-androstan-17-one, 16β-chloro-3β-hydroxy-5α-androstan-17-one, 16α,3β-dihydroxy-5α-androstan-17-one, 16β,3β-dihydroxy-5α-androstan-17-one, 16α,3α-dihydroxy-5α-androstan-17-one, 16β,3α-dihydroxy-5α-androstan-17-one, 16α-bromo-3β-hydroxy-5α-androstan-17-one hemihydrate, 3α-hydroxy-16α-fluoroandrostane-17-one, 3β-hydroxy-16α-fluoroandrostane-17-one, 17α-hydroxy-16α-fluoroandrostane-3-one, 17β-hydroxy-16α-fluoroandrostane-3-one, 17α-hydroxy-16α-fluoroandrostane-4-one, 17β-hydroxy-16α-fluoroandrostane-4-one, 17α-hydroxy-16α-fluoroandrostane-6-one, 17β-hydroxy-16α-fluoroandrostane-6-one, 17α-hydroxy-16α-fluoroandrostane-7-one, 17β-hydroxy-16α-fluoroandrostane-7-one, 17α-hydroxy-16α-fluoroandrostane-11-one, 17β-hydroxy-16α-fluoroandrostane-11-one, 16α-fluoroandrost-5-ene-17-one, 7α-hydroxy-16α-fluoroandrost-5-ene-17-one, 7β-hydroxy-16α-fluoroandrost-5-ene-17-one, 4α-hydroxy-16α-fluoroandrost-5-ene-17-one, 3α-hydroxy-16α-fluoroandrost-5-ene-17-one, 3β-hydroxy-16α-fluoroandrost-5-ene-17-one,4β-hydroxy-16α-fluoroandrost-5-ene-17-one, 6α-hydroxy-16α-fluoroandrost-5-ene-17-one, 6β-hydroxy-16α-fluoroandrost-5-ene-17-one, 11α-hydroxy-16α-fluoroandrost-5-ene-17-one, 11β-hydroxy-16α-fluoroandrost-5-ene-17-one, 4α,17β-dihydroxy-16α-fluoroandrost-5-ene, 4β,17β-dihydroxy-16α-fluoroandrost-5-ene, 6α,17β-dihydroxy-16α-fluoroandrost-5-ene, 6β,17β-dihydroxy-16α-fluoroandrost-5-ene, 11α,17β-dihydroxy-16α-fluoroandrost-5-ene, 11β,17β-dihydroxy-16α-fluoroandrost-5-ene, 4α,17α-dihydroxy-16α-fluoroandrost-5-ene, 4β,17α-dihydroxy-16α-fluoroandrost-5-ene, 6α,17α-dihydroxy-16α-fluoroandrost-5-ene, 6β,17α-dihydroxy-16α-fluoroandrost-5-ene, 11α,17α-dihydroxy-16α-fluoroandrost-5-ene, 11β,17α-dihydroxy-16α-fluoroandrost-5-ene, 7α,17β-dihydroxy-16α-fluoroandrost-5-ene, 7β,17β-dihydroxy-16α-fluoroandrost-5-ene, 3α,17β-dihydroxy-16α-fluoroandrost-5-ene, 3β,17β-dihydroxy-16α-fluoroandrost-5-ene, 3α,17α-dihydroxy-16α-fluoroandrost-5-ene, 3β,17α-dihydroxy-16α-fluoroandrost-5-ene, 1α,17β-dihydroxy-16α-fluoroandrost-5-ene, 1β,17β-dihydroxy-16α-fluoroandrost-5-ene, 2α,17β-dihydroxy-16α-fluoroandrost-5-ene, 2β,17β-dihydroxy-16α-fluoroandrost-5-ene, 12α,17β-dihydroxy-16α-fluoroandrost-5-ene, 12β,17β-dihydroxy-16α-fluoroandrost-5-ene, 1α,17α-dihydroxy-16α-fluoroandrost-5-ene, 1β,17α-dihydroxy-16α-fluoroandrost-5-ene, 2α,17α-dihydroxy-16α-fluoroandrost-5-ene, 2β,17α-dihydroxy-16α-fluoroandrost-5-ene, 12α,17α-dihydroxy-16α-fluoroandrost-5-ene, 12β,17α-dihydroxy-16α-fluoroandrost-5-ene, 15α,17β-dihydroxy-16α-fluoroandrost-5-ene, 15β,17β-dihydroxy-16α-fluoroandrost-5-ene, 17β,18-dihydroxy-16α-fluoroandrost-5-ene, 17β,19-dihydroxy-16α-fluoroandrost-5-ene, 15α,17α-dihydroxy-16α-fluoroandrost-5-ene, 15β,17α-dihydroxy-16α-fluoroandrost-5-ene, 17α,18-dihydroxy-16α-fluoroandrost-5-ene, 17α,19-dihydroxy-16α-fluoroandrost-5-ene, 16α-fluoroandrost-4-ene-17-one, 7α-hydroxy-16α-fluoroandrost-4-ene-17-one, 7β-hydroxy-16α-fluoroandrost-4-ene-17-one, 3α-hydroxy-16α-fluoroandrost-4-ene-17-one, 3β-hydroxy-16α-fluoroandrost-4-ene-17-one, 4α-hydroxy-16α-fluoroandrost-4-ene-17-one, 4β-hydroxy-16α-fluoroandrost-4-ene-17-one, 6α-hydroxy-16α-fluoroandrost-4-ene-17-one, 6β-hydroxy-16α-fluoroandrost-4-ene-17-one, 11α-hydroxy-16α-fluoroandrost-4-ene-17-one, 11β-hydroxy-16α-fluoroandrost-4-ene-17-one, 4α,17β-dihydroxy-16α-fluoroandrost-4-ene, 4β,17β-dihydroxy-16α-fluoroandrost-4-ene, 6α,17β-dihydroxy-16α-fluoroandrost-4-ene, 6β,17β-dihydroxy-16α-fluoroandrost-4-ene, 11α,17β-dihydroxy-16α-fluoroandrost-4-ene, 11β,17β-dihydroxy-16α-fluoroandrost-4-ene, 4α,17α-dihydroxy-16α-fluoroandrost-4-ene, 4β,17α-dihydroxy-16α-fluoroandrost-4-ene, 6α,17α-dihydroxy-16α-fluoroandrost-4-ene, 6β,17α-dihydroxy-16α-fluoroandrost-4-ene, 11α,17α-dihydroxy-16α-fluoroandrost-4-ene, 11β,17α-dihydroxy-16α-fluoroandrost-4-ene, 7α,17β-dihydroxy-16α-fluoroandrost-4-ene, 7β,17β-dihydroxy-16α-fluoroandrost-4-ene, 3α,17β-dihydroxy-16α-fluoroandrost-4-ene, 3β,17β-dihydroxy-16α-fluoroandrost-4-ene, 3α,17α-dihydroxy-16α-fluoroandrost-4-ene, 3β,17α-dihydroxy-16α-fluoroandrost-4-ene, 1α,17β-dihydroxy-16α-fluoroandrost-4-ene, 1β,17β-dihydroxy-16α-fluoroandrost-4-ene, 2α,17β-dihydroxy-16α-fluoroandrost-4-ene, 2β,17β-dihydroxy-16α-fluoroandrost-4-ene, 12α,17β-dihydroxy-16α-fluoroandrost-4-ene, 12β,17β-dihydroxy-16α-fluoroandrost-4-ene, 1α,17α-dihydroxy-16α-fluoroandrost-4-ene, 1β,17α-dihydroxy-16α-fluoroandrost-4-ene, 2α,17α-dihydroxy-16α-fluoroandrost-4-ene, 2β,17α-dihydroxy-16α-fluoroandrost-4-ene, 12α,17α-dihydroxy-16α-fluoroandrost-4-ene, 12β,17α-dihydroxy-16α-fluoroandrost-4-ene, 15α,17β-dihydroxy-16α-fluoroandrost-4-ene, 15β,17β-dihydroxy-16α-fluoroandrost-4-ene, 17β,18-dihydroxy-16α-fluoroandrost-4-ene, 17β,19-dihydroxy-16α-fluoroandrost-4-ene, 15α,17α-dihydroxy-16α-fluoroandrost-4-ene, 15β,17α-dihydroxy-16α-fluoroandrost-4-ene, 17α,18-dihydroxy-16α-fluoroandrost-4-ene, 17α,19-dihydroxy-16α-fluoroandrost-4-ene, 3β,17β-dihydroxyandrost-5-ene, 3β-hydroxy-7,17-dioxoandrost-5-ene, 3α-hydroxy-7,17-dioxoandrost-5-ene, 3,17-dioxoandrost-5-ene, 3,17-dioxoandrost-4-ene, 3,17-dioxoandrost-1,4-diene, 3β,7β,17β-trihydroxyandrost-5-ene, 3β,7β,17β-trihydroxyandrostane, 3β,16α-dihydroxy-17-oxoandrostane, 3α,16α-dihydroxy-17-oxoandrostane, 3β,16β-dihydroxy-17-oxoandrostane, 3α,16β-dihydroxy-17-oxoandrostane, 3β,16α,17β-trihydroxyandrostane, 3β,16β,17β-trihydroxyandrostane, 3β,16α,17α-trihydroxyandrostane, 3β,16β,17α-trihydroxyandrostane, 3α,16α,17β-trihydroxyandrostane, 3α,16β,17β-trihydroxyandrostane or an analog of any of these compounds that is (1) 11-oxa substituted or 2-oxa substituted if no double bond is present at the 1-2 position, (2) substituted at the 7-position with an α-halogen, β-halogen, α-hydroxyl, β-hydroxyl or oxo moiety, (3) a D-ring homo analog, (4) a 19-nor analog and/or (5) an analog of any of the foregoing compounds that is substituted with an R¹⁰ substituent disclosed herein, e.g., wherein the R¹⁰ is a hydroxyl, thiol, —NH₂, —NH— optionally substituted alkyl, optionally substituted alkyl or an independently selected halogen such as fluorine, chlorine or bromine at 1, 2, 3 or more of the 1-, 2-, 4-, 6-, 9-11-, 12-, 14-, 15- or 16-positions, wherein the R¹⁰, e.g., the hydroxyl, thiol, optionally substituted alkyl or halogen is present in the α-configuration or the β-configuration.

30. A method to characterize a test compound or treatment comprising, (1) providing an exposed subject who has been exposed to a biological insult optionally selected from a radiation dose of at least about an LD₅; (2) measuring one, two or more of the exposed subject's biological parameters or biological responses to the radiation exposure to obtain the exposed subject's status profile, wherein the exposed subject's status profile indicates (i) that the subject has a probability of at least about 50% of not surviving the exposure to the radiation dose or (ii) the subject will not survive the biological insult with a P_{lethality} of 0.10, 0.5 or less; (3) administering the test compound to the subject; (4) optionally determining the efficacy of the test compound, wherein efficacy is optionally measured as one, two or more of survival of the exposed subject, prolonged survival of the exposed subject, decreased susceptibility to infection, amelioration of pain or suffering of the exposed subject, reduced need for other therapeutic treatments, wherein the other therapeutic treatments are optionally selected from one, two, three or more blood transfusions, platelet transfusions, red cell transfusions and transfusions with another blood type or blood product.

31. The method of embodiment 30 wherein the biological insult is a radiation dose of about an LD₁₀, about an LD₂₀, about an LD₃₀, about an LD₄₀, about an LD₅₀, about an LD₆₀, about an LD₇₀, about an LD₈₀, about an LD₉₀ or about an LD₁₀₀, where survival is measured at 30 days or at 60 days, or wherein the radiation dose is about 2 Gy to about 10 Gy, or wherein the radiation dose is about 6 Gy, where survival is optionally measured at 30 days or at 60 days after the radiation exposure.

32. The method of embodiment 30 wherein the biological insult is a chemotherapy of about an LD₁₀, about an LD₂₀, about an LD₃₀ or about an LD₄₀, where survival is optionally measured at 30 days or at 60 days after the chemotherapy.

33. The method of embodiment 30, 31 or 32 wherein the test compound is a protein, a steroid, a steroid analog, a statin or a molecule having a molecular weight of about 70 da to about 2,500 da.

34. A method to facilitate obtaining or to obtain (i) regulatory approval to initiate a human clinical trial, an animal clinical trial or to market a new pharmaceutical or veterinary drug, drug treatment protocol or device or to (ii) facilitate regulatory review of a human clinical trial, an animal clinical trial or regulatory review of an application to market a new pharmaceutical or veterinary drug, drug treatment protocol or device, the method comprising, (1) obtaining a Pₛᵤᵣᵥᵢᵥₐₗ or P_{lethality} status profile for a human, a non-human primate or another mammal, wherein the Pₛᵤᵣᵥᵢᵥₐₗ or P_{lethality} status profile predicts survival or death with at least about a 80% degree of confidence or P≧0.8; and (2) submitting the Pₛᵤᵣᵥᵢᵥₐₗ or P_{lethality} status profile to an appropriate government or regulatory agency.

35. The method of embodiment 34 wherein the Pₛᵤᵣᵥᵢᵥₐₗ or P_{lethality} status profile is used as a surrogate for lethality.

36. The method of embodiment 34 or 35 wherein the Pₛᵤᵣᵥᵢᵥₐₗ or P_{lethality} status profile is for a non-human primate and optionally wherein the regulatory approval or regulatory review is for a new human drug, treatment protocol or device.

37. The method of embodiment 34, 35 or 36 wherein the appropriate government or regulatory agency is the U.S. Food and Drug Administration, the U.S. Department of Energy or the U.S. Department of Defense.

38. The method of embodiment 34, 35, 36 or 37 wherein the Pₛᵤᵣᵥᵢᵥₐₗ or P_{lethality} status profile predicts survival or death with (i) at least about a 90% degree of confidence or P≧0.90 or (i) at least about a 95% degree of confidence or P≧0.95.

39. The method of embodiment 34, 35, 36, 37 or 38 wherein the Pₛᵤᵣᵥᵢᵥₐₗ or P_{lethality} status profile is for radiation exposure, optionally wherein the radiation dose is about an LD₁₀, about an LD₂₀, about an LD₃₀, about an LD₄₀, about an LD₅₀, about an LD₆₀, about an LD₇₀, about an LD₈₀, about an LD₉₀ or about an LD₁₀₀, where survival is measured at 30 days or at 60 days, or wherein the radiation dose is about 2 Gy to about 10 Gy, or wherein the radiation dose is about 6 Gy.

40. The method of embodiment 39 wherein the radiation exposure is γ-radiation, X-rays, β-radiation, fast neutrons and slow neutrons, optionally selected from the group consisting of ⁶⁰Co, ¹²⁷Cs radiation, a radioactive iodine isotope, and optionally wherein the dose rate is about 50 cGy/minute, about 60 cGy/minute or about 70 cGy/minute.

41. The method of embodiment 34, 35, 36, 37, 38, 30 or 40 wherein the regulatory approval is for (i) initiatiation or review a human clinical trial for a new drug or (ii) initiatiation or review a human clinical trial for a new clinical indication or treatment protocol for an existing approved drug.

42. The method of embodiment 41 wherein the new drug is a formula 1 compound.

43. The method of 42 wherein the formula 1 compound is (i) 3β-hydroxy-17β-aminoandrost-5-ene, 3β-hydroxy-17β-aminoandrost-4-ene, 3β-hydroxy-17β-aminoandrost-1-ene, 3α-hydroxy-17β-aminoandrost-5-ene, 3α-hydroxy-17β-aminoandrost-4-ene, 3α-hydroxy-17β-aminoandrost-1-ene, 3β-hydroxy-17β-aminoandrost-1,5-diene, 3β-hydroxy-17β-aminoandrost-1,4-diene or a 19-nor, 2-oxa, 11-oxa, 2α-hydroxy, 2β-hydroxy, 2α-alkoxy, 2β-alkoxy, 7β-hydroxy, 7β-optionally substituted alkyl, 11α-optionally substituted alkyl, 11β-optionally substituted alkyl, 17β-optionally substituted alkylamino (where —NH-optionally substituted alkyl is present at the 17-position), 17β-di(optionally substituted alkylamino) (where —NH-(optionally substituted alkyl)₂ is present at the 17-position), 3α-optionally substituted alkyl and/or 3β-optionally substituted alkyl analog of any of these compounds, wherein the optionally substituted alkyl groups independently optionally contain 1, 2, 3, 4, 5 or 6 carbon atoms and/or 1 or 2 double bonds and/or triple bonds and wherein all optionally substituted alkyl groups are independently chosen, or (ii) any formula 1 compound described in any preceeding embodiment, compound group or chemical structure described anywhere herein.

44. The method of 34, 35, 36, 37, 38, 39, 40, 41, 42 or 43 wherein the regulatory approval or review is for a drug or a treatment protocol to treat, prevent or ameliorate one or more biological effects of radiation exposure.

45. The method of 33, 34, 35, 36, 37, 38, 39, 40, 41, 42, 43 or 44 wherein the regulatory approval or review is by the U.S. Food and Drug Administration, optionally wherein the regulatory approval or review is at least in part specified, defined or regulated by one, two or more of the statutes or regulations at 21 U.S.C. § 360e, 21 U.S.C. § 355(j)(2)(A)(vii)(I)-(IV), 21 U.S.C. § 355(j)(2)(B), 21 U.S.C. § 351, 21 U.S.C. § 352, 21 U.S.C. § 353, 21 C.F.R. § 314, 21 C.F.R. §§ 314, 314.600, 314.610, 314.620, 314.630, 21 C.F.R. § 600, 21 C.F.R. §§ 601, 601.90, 601.91, 601.92, 601.93.

46. A kit for measuring temperature in a subject that has been exposed to a biological insult comprising (i) one, two or a plurality of temperature measuring devices, e.g., about 5, 10, 15, 20, 25, 30, 40, 50 or more, and (ii) instructions that directs use of the temperature measuring devices so as to (a) detect a fever or elevated temperature of at least about 0.5° C. or at least about 0.1° C. above a baseline or normal human temperature, optionally (b) within a time period of about 15 minutes to about 24 hours after a biological insult as described in any embodiment or elsewhere herein and/or (c) optionally wherein when the fever or elevated temperature is at least about 0.8° C. or at least about 1.0° C. above baseline or normal human temperature or where the the fever elevated temperature remains elevated above baseline or normal human temperature at least about 0.8° C. or by at least about 1.0° C. for at least about 15 minutes to about 24 hours, the elevated temperature (1) corresponds with a P_{lethality} or probability that the subject will survive biological insult of less than about 0.1 or less than about 0.05, or (2) the biological insult has a probability that is greater than about 50%, about 60% or about 70% of causing a life-threatening adverse or toxic biological response.

47. The kit of embodiment 46 wherein the temperature measuring devices are single use devices, optionally wherein the devices monitor only peak temperature during the period in which the temperature measuring devices are in use, optionally wherein the peak temperature is indicated by a color change or by a highlighted numeric temperature value.

48. The kit of embodiment 46 or 47 wherein the kit is subject to regulatory review or to regulatory approval, optionally wherein the regulatory approval or review is by the U.S. Food and Drug Administration, optionally wherein the regulatory approval or review is at least in part specified, defined or regulated by one, two or more of the statutes or regulations at 21 U.S.C. § 360e, 21 U.S.C. § 355(j)(2)(A)(vii)(I)-(IV), 21 U.S.C. § 355(j)(2)(B), 21 U.S.C. § 351, 21 U.S.C. § 352, 21 U.S.C. § 353, 21 C.F.R. § 314, 21 C.F.R. §§ 314, 314.600, 314.610, 314.620, 314.630 and 21 C.F.R. § 600, 21 C.F.R. §§ 601, 601.90, 601.91, 601.92, 601.93.

49. The kit of embodiment 46, 47 or 48 wherein the biological insult is a radiation exposure, optionally wherein the radiation exposure is from one or more radiation sources described herein such as γ-radiation, X-radiation or an ionizing neutron radiation, wherein (a) the dose and/or doase rate of the radiation exposure is not known or (b) the dose and/or doase rate of the radiation exposure is known.

50. The kit of embodiment 46, 47, 48 or 49 wherein the temperature measuring devices are used by affixing the temperature measuring devices to the skin, e.g., by an adhesive layer or material attached to the temperature measuring devices, or by manually holding or otherwise maintaining the temperature measuring devices in contact with the skin for sufficient time to allow measurement of skin temperature, but where such manually holding or maintaining skin contact does not substantially or detectably interfere with the temperature measurement.

51. The kit of embodiment 46, 47, 48, 49 or 50 wherein the instructions that direct the use of the temperature measuring devices specifiy (i) that the temperature measuring devices are to be brought into contact with the skin for sufficient time to allow measurement of skin temperature, (ii) optionally followed by removal of the temperature measuring devices, and (iii) recording of the numerical temperature or notation of one or more temperature measuring devices whose color change indicates that the subject's temperature is elevated.

52. A method comprising measuring one, two6three or more surrogate markers for death or survival in a subject that has been exposed to a biological insult of at least about an LD₅ and optionally treating the subject with an ameliorative or palliative treatment. In these embodiments, the surrogate markers are optionally selected from (i) the duration of febrile severe neutropenia or the duration of severe neutropenia, (ii) duration of severe thrombocytopenia, (iii) time, e.g., delay, of onset of febrile severe neutropenia or severe neutropenia, (iv) time, e.g., delay, of onset of severe thrombocytopenia, (v) degree of severity of febrile severe neutropenia or severe neutropenia, or (vi) degree of severity of severe neutropenia. The subjects can be humans, non-human primates or other subjects described herein.

53. The method of embodiment 52 wherein the biological insult is ionizing radiation, trauma, toxin exposure or ingestrion or a chemotherapy. The biological insult can be about an LD₁₀, LD₂₀, LD₃₀ or LD₄₀ to about an LD₅₀, LD₆₀, LD₇₀ or LD₁₀₀. More severe biological insults, e.g., exposure to an ionizing radiation dose of about 1.5-fold, 2-fold or 5-fold above an LD₁₀₀ can also be used.

54. The method of embodiment 52 or 53 wherein the subject is treated with a formula 1 compound and/or another compound or treatment as described herein, optionally wherein the subject is assessed for the effect of the compound or treatment on the onset time of febrile severe neutropenia, severe neutropenia, severe thrombocytopenia or another surrogate marker described herein. In some embodiments, other treatments include administering blood, platelets or other blood products to the subject and/or administering an one, two or more antibiotics to the subject. Such treatments can be used to prevent or delay the onset of an unwanted side-effect or toxicity associated with the biological insult or to ameliorate an existing side-effect or toxicity, e.g., an infection or severe thrombocytopenia.

55. The method of embodiment 52 or 53 wherein the subject is treated with a formula 1 compound and no other compound or treatment other than maintaining the subject, e.g., by administering food or liquid to the subject as needed or desired. In these embodiments, the effect of the treatment with the formula 1 compound can be observed as delay of time of onset of febrile severe neutropenia, severe neutropenia, severe thrombocytopenia or another surrogate marker described herein. In non-human primates or humans for example, the time from exposure to the biological insult to the onset or occurrence of, e.g., febrile severe neutropenia, severe neutropenia, severe thrombocytopenia, circadian rhythm disruption or other conditions can occur beginning at about 13, 14, 15 or 16 days after exposure of the subjects to the biological insult. For exposed subjects that have been treated with a formula 1 compound the onset of such conditions can be prevented completely or delayed to begin at about 15, 16,17, 18,19 or 20 days after exposure to the biological insult in subjects that have been treated with a formula 1 compound. Typically, the treatment with the formula 1 compound will begin at about 10 minutes to about 96 hours after exposure to the biological insult. For exposure to ionizing radiation, a pathogen such as Bacillus anthracis or smallpox virus or for trauma, treatment with the formula 1 compound will typically begin at about 10 or 30 minutes to about 4, 12 or 24 hours after exposure, e.g., treatment can begin at about 15 minutes, 30 minutes, 1 hour, 2 hours, 3 hours, 4 hours, 5 hours, 6 hours, 8 hours, 10 hours, 12 hours, 14 hours, 16 hours or 18 hours after the exposure or trauma. For exposure to a toxin or chemotherapy, treatment with the formula 1 compound will typically begin at about 12, 24, 28, 30, 36 40, 44, 48, 60 or 72 hours after exposure. Treatment with the formula 1 compound will be maintained for 1 or 2 days to about 7, 10, 14 or 21 days by daily dosing or by intermittent dosing.

56. The method of embodiment 52, 53, 54 or 55 wherein (1) about 1 mg/kg/day to about 50 mg/kg/day of the formula 1 compound is administered to the subject or (2) about 0.1 mg/day to about 2500 mg/day of the formula 1 compound is administered to the subject, e.g., about 20 or 40 mg/day to about 100, 200, 400 or 1000 mg/day for humans or non-human primates or (3) sufficient amounts of the formula 1 compound is administered to obtain a blood or serum level of about 0.5, 1, 2 or 5 ng/mL to about 8, 10.20, 100, 200 or 500 ng/mL of the formula 1 compound or a metabolite of the formula 1 compound where the level is reached at least transiently, e.g., for about 5 minutes per day to about 30 minutes per day or for longer periods, e.g., for about 1 or 2 hours per day to about 3, 4, 6, 12 or more hours per day, e.g., on days when the formula 1 compound is administered to the subject or on one, two, three or more days after the formula 1 compound is administered to the subject. Small doses, e.g., 0.1 or 1 mg/day to about 2, 3 or 5 mg/day, will typically be used for small subjects, e.g., mice or rats. Larger doses such as about 500 mg/day or 1200 mg/day will typically be administered to larger subjects, e.g., humans or non-human primates, and/or administered orally.

57. A method to evaluate the capacity of a compound to (i) increase the survival rate or probability of survival or (ii) enhance the survival experience of a subject that has been exposed to a biological insult such as a radiation dose of about an LD_{50/30} or LD_{50/60}, by treating the exposed subject with an effective amount of the compound, wherein the compound optionally has the structure

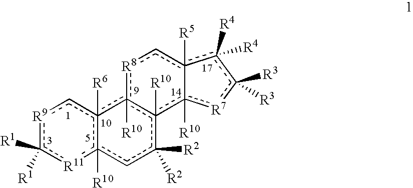

or a metabolic precursor, a metabolite, salt or tautomer thereof, wherein the dotted lines are optional double bonds and 0, 1, 2, 3, 4 or 5 double bonds are present in the steroid rings, each R¹, R², R³, R⁴, R⁵, R⁶ and R¹⁰ independently or together are —H, —OH, —OR^{PR}, —SR^{PR}, —SH, —N(R^{PR})₂, —NHR^{PR}, —NH₂, —O—Si—(R¹³)₃, —CHO, —CHS, —CN, —SCN, —NO₂, —N₃, —COOH, —COOR^{PR}, —OSO₃H, —OSO₂H, —OPO₃H₂, ═O, ═S, ═N—OH, ═N—OCH₃, ═CH₂, ═CH—CH₃, ═CH-optionally substituted alkyl, ester, thioester, thionoester, phosphoester, phosphothioester, phosphonate, phosphonate ester, thiophosphonate, thiophosphonate ester, phosphiniester, sulfite ester, sulfate ester, sulfamate, sulfonate, sulfonamide, amide, amino acid, peptide, ether, thioether, acyl, thioacyl, carbonate, carbamate, halogen, optionally substituted alkyl, optionally substituted alkenyl, optionally substituted alkynyl, optionally substituted aryl, optionally substituted heteroaryl, optionally substituted heterocycle, optionally substituted monosaccharide, optionally substituted oligosaccharide, polymer, spiro ring, epoxide, acetal, thioacetal, ketal, thioketal, —S—S-optionally substituted alkyl, ═N—O-optionally substituted alkyl, ═N-optionally substituted alkyl, —NH-optionally substituted alkyl, —NH—S(O)(O)-optionally substituted alkyl, —N(optionally substituted alkyl)₂ where each optionally substituted alkyl is independently selected, or, one or more of two adjacent R¹, R², R³, R⁴, R⁵, R⁶ and R¹⁰ comprise an independently selected epoxide or optionally substituted saturated or unsaturated cyclopropyl, cyclobutyl, cyclopental or cyclohexyl ring any of which rings optionally contain a ring heteroatom such as —O—, —S—, —NH— or ═N—; R⁷ is —O—, —S—, —S(O)(O)—, —NR^{PR}—, —C(R¹⁰)₂—, —C(R¹⁰)₂—C(R¹⁰)₂—, —C(R¹⁰)₂-C(R¹⁰)₂—C(R¹⁰)₂—, —C(R¹⁰)₂—O—C(R¹⁰)₂—, —C(R¹⁰)₂—S—C(R¹⁰)₂—, —C(R¹⁰)₂—NR^{PR}—C(R¹⁰)₂—, —O—C(R¹⁰)₂—, —S—C(R¹⁰)₂— or —NR^{PR}—C(R¹⁰)₂—, where each R¹⁰ is independently selected; R⁸ and R⁹ independently are —C(R¹⁰)₂—, —C(R¹⁰)₂—C(R¹⁰)₂—, —O—, —O—C(R¹⁰)₂—, —S—, —S(O)(O)—, —S—C(R¹⁰)₂—, —S(O)(O)—C(R¹⁰)₂—, —NR^{PR}— or —NR^{PR}—C(R¹⁰)₂—, or one or both of R⁸ or R⁹ independently are absent, leaving a 5-membered ring, where each R¹⁰ is independently selected; R¹¹ is —O—, —S—, —S(O)(O)—, —NR^{PR}—, —CH₂—, —CHR¹⁰—, —C(R¹⁰)₂—, —C(R¹⁰)₂—O—C(R¹⁰)₂—, —C(R¹⁰)₂—S—C(R¹⁰)₂—, —C(R¹⁰)₂—S(O)(O)—C(R¹⁰)₂—, —C(R¹⁰)₂—NR^{PR}—C(R¹⁰)₂—, —O—C(R¹⁰)₂—, —S—C(R¹⁰)₂—, —S(O)(O)—C(R¹⁰)₂— or —NR^{PR}—C(R¹⁰)₂—, where each R¹⁰ is independently selected; R¹³ independently is C₁₋₆ alkyl; R^{PR} independently are —H or a protecting group; and optionally wherein one, two or three of the 1-, 4-, 6- and/or 12-positions are optionally substituted with (i) an independently selected R¹⁰ moiety when a double bond is present at the corresponding 1-, 4-, 6- or 12-position, or (ii) one or two independently selected R¹⁰ moieties when no double bond is present at the corresponding 1-, 4-, 6- and/or 12-position.

58. The method of embodiment 57 wherein the compound is 3β,17β-dihydroxyandrost-5-ene, 3α,17β-dihydroxyandrost-5-ene, 3β-hydroxy-17β-mercaptoandrost-5-ene, 3β-hydroxy-17β-aminoandrost-5-ene, 3β,17β-dihydroxyandrost-5(10)-ene, 3α,17β-dihydroxyandrost-5(10)-ene, 3β-hydroxy-17β-mercaptoandrost-5(10)-ene, 3β-hydroxy-17β-aminoandrost-5(10)-ene or an analog of any of these compounds comprising a carbonate, ester, ether, thioester, thioether, disulfide, carbamate, amino acid, monosaccharide, sulfate, phosphate, polymer or amide such as —NH—C(O)-optionally substituted alkyl derivative of any of the —OH, —SH or —NH₂ groups in these compounds, where the optionally substituted alkyl moiety optionally contains 1, 2, 3, 4, 5, 6, 7 or 8 carbon atoms.

59. The method of embodiment 57 or 58 wherein the subject is a human or a non-human primate and the biological insult is exposure to ionizing radiation, chemotherapy or trauma.

60. The method of embodiment 57, 58 or 59 where, after exposure to the biological insult, the subject (i) is or (ii) is not treated with another ameliorative or palliative treatment(s) such as one, two or more of analgesic treatment or pain management therapy, blood, fluid or plasma transfusion, cord blood transfusion, hormone treatment such as androgen or estrogen treatment, antibiotic treatment, cytokine treatment such as administration of GCS-F, GMCS-F, thrombopoietin, erythropoietin or IL-2, administration of cells such as stem cells.

61. A method to (i) increase the survival rate or probability of survival or (ii) enhance the survival experience of a subject that has been exposed to a biological insult such as a radiation dose of at least about an LD_{5/30} or LD_{5/60}, comprising treating the exposed subject with an effective amount of the compound of embodiment 58 or 59, wherein the increase or enhancement is relative to a suitable control subject(s), such as subjects of the same species or of a closely related species that have been exposed to the same or a similar or a comparable biological insult and wherein the control subjects were exposed to the same, similar or comparable biological insult at about the same time or at an earlier time such as several months or years earlier.

62. The method of embodiment 61 wherein the survival rate is increased by at least about 5%, at least about 10%, at least about 15%, at least about 20% or at least about 25%, to at least about 30%, at least about 35%, at least about 40%, at least about 45%, at least about 50%, at least about 60%, at least about 70%, at least about 80%, at least about 90% or about 100%.

63. The method of embodiment 61 or 62 wherein the survival rate is increased by about 5%, about 10% or about 15% to about 20%, about 30%, about 70% or about 80% or more compared to a suitable control subject not treated with the compound but exposed to the same or essentially the same or a comparable biological insult.

64. The method of embodiment 61, 62 or 63 wherein the biological insult is a radiation exposure.

65. The method of embodiment 61, 62, 63 or 64 wherein the biological insult is a radiation dose that results in at least about an LD_{10/30} or LD_{10/60}, at least about an LD_{20/30} or LD_{20/60}, at least about an LD_{30/30} or LD_{30/60}, at least about an LD_{40/30} or LD_{40/60}, at least about an LD_{50/30} or LD_{50/60}, at least about an LD_{60/30} or LD_{60/60}, at least about an LD_{70/30} or LD_{70/60}, at least about an LD_{80/30} or LD_{80/60}, at least about an LD_{90/30} or LD_{90/60}.

66. The method of embodiment 61, 62, 63, 64 or 65 wherein the biological insult is a radiation dose that results in about an LD_{10/30} or LD_{10/60}, about an LD_{20/30} or LD_{20/60}, about an LD_{30/30} or LD_{30/60}, about an LD_{40/30} or LD_{40/60}, about an LD_{50/30} or LD_{50/60}, about an LD_{60/30} or LD_{60/60}, about an LD_{70/30} or LD_{70/60}, about an LD_{80/30} or LD_{80/60}, about an LD_{90/30} or LD_{90/60}, about an LD_{95/30} or LD_{95/60} or about an LD_{100/30} or LD_{100/60}.

67. The method of embodiment 61, 62, 63, 64, 65 or 66 wherein the enhanced survival experience is an increase in the time from exposure to the biological insult to one, two three or more of (1) death, (2) delay of onset of severe neutropenia such as an absolute neutrophil count in humans or in Rhesus monkeys of less than 500 cells per μL when compared to controls not treated with the compound, (3) delay of onset of severe thrombocytopenia such as a platelet count in humans or in Rhesus monkeys of less than 10,000 per μL when compared to controls not treated with the compound, (4) delay of onset of anemia such as a reduction in red blood cell count or hematocrit or hemoglobin level of at least about 20% in humans or in Rhesus monkeys of when compared to controls not treated with the compound, (5) reduction in the number of days the subject has severe neutropenia, (6) reduction in the number of days the subject has severe thrombocytopenia, (7) reduction in the number of days the subject has anemia, (8) amelioration or reduction in the temperature or fever spike that occurs within about 12-16 hours after exposure to the biological insult, such as a reduction of about 0.4° C., about 0.6° C., about 0.8° C. or more, (9) reduction in the occurrence or severity of sepsis, (10) reduction in the occurrence or severity of hemorrhagic sepsis, (11) reduction in the severity or duration of fever beginning at about 13, 14 or 15 days after the biological insult to about 20, 25, 30 or 35 days after the biological insult, (12) reduction in the occurrence or severity of internal bleeding, (13) delay of onset of grade 2 or 3 neutropenia, (14) reduction in the duration of grade 2 or 3 neutropenia, (15) delay of onset of grade 2 or 3 thrombocytopenia, (16) reduction in the duration of grade 2 or 3 thrombocytopenia, (17) delay of onset of febrile severe neutropenia such as a core body temperature of >40.4° C. and an absolute neutrophil count of <500 cells/μL in a human or a non-human primate, (18) reduced duration of febrile severe neutropenia such as a core body temperature of >40.4° C. and an absolute neutrophil count of <500 cells/μL in a human or a non-human primate and (19) bone marrow hyopcellularity or disruption.

68. The method of embodiment 61, 62, 63, 64, 65, 66 or 67 wherein the subject is a human and the control subject(s) is the rhesus monkey, cynomolgus monkey or baboon.

69. The method of embodiment 61, 62, 63, 64, 65, 66, 67 or 68 wherein the compound is 3α,17β-dihydroxyandrost-5-ene, 3α,17β-dihydroxyandrost-5-ene, 3β-hydroxy-17β-mercaptoandrost-5-ene, 3α-hydroxy-17β-mercaptoandrost-5-ene or an analog of any of these compounds wherein (i) the analog is an ester, ether, thioester and/or thioether derivative of one or two hydroxyl or mercapto groups, (ii) a double bond is present at the 1-, 4-, and/or 5(10)-position, (iii) the analog is a 19-nor derivative, and/or (iv) an O-linked, S-linked or N-linked moiety is bonded to the 16-position and/or the 7-position wherein the O-linked, S-linked or N-linked moiety is in the α- or β-configuration if single bonded.

70. A method comprising (i) administering a test compound(s) to or conducting a test therapy on a non-human primate(s) that has been exposed to a radiation dose such as about an LD_{10/30}, about an LD_{20/30} or an LD_{50/30} of γ-radiation or X-radiation to obtain an exposed subject(s); (ii) measuring a biological response associated with the test compound(s) or test therapy selected from one, two, three or more of (1) death, (2) delay of onset of severe neutropenia such as an absolute neutrophil count in humans or in Rhesus monkeys of less than 500 cells per μL when compared to controls not treated with the compound, (3) delay of onset of severe thrombocytopenia such as a platelet count in humans or in Rhesus monkeys of less than 10,000 per μL when compared to controls not treated with the compound, (4) delay of onset of anemia such as a reduction in red blood cell count or hematocrit or hemoglobin level of at least about 20% in humans or in Rhesus monkeys of when compared to controls not treated with the compound, (5) reduction in the number of days the subject has severe neutropenia, (6) reduction in the number of days the subject has severe thrombocytopenia, (7) reduction in the number of days the subject has anemia, (8) amelioration or reduction in the temperature or fever spike that occurs within about 12-16 hours after exposure to the biological insult, such as a reduction of about 0.4° C., about 0.6° C., about 0.8° C. or more, (9) reduction in the occurrence or severity of sepsis, (10) reduction in the occurrence or severity of hemorrhagic sepsis, (11) reduction in the severity or duration of fever beginning at about 13, 14 or 15 days after the biological insult to about 20, 25, 30 or 35 days after the biological insult, (12) reduction in the occurrence or severity of internal bleeding, (13) delay of onset of grade 2 or 3 neutropenia, (14) reduction in the duration of grade 2 or 3 neutropenia, (15) delay of onset of grade 2 or 3 thrombocytopenia, (16) reduction in the duration of grade 2 or 3 thrombocytopenia, (17) delay of onset of febrile severe neutropenia such as a core body temperature of >40.4° C. and an absolute neutrophil count of <500 cells/μL in a human or a non-human primate, (18) reduced duration of febrile severe neutropenia such as a core body temperature of >40.4° C. and an absolute neutrophil count of <500 cells/μL in a human or a non-human primate and (19) bone marrow hyopcellularity; and (iii) comparing the biological response of the test compound or test therapy in the exposed subject(s) with the same or a comparable biological response from a control exposed subject(s) of the same or a closely related species that has been treated with (a) a compound of embodiment 57 or 58 or a F1C described herein, or where the compound is 3β,17β-dihydroxyandrost-5-ene.

71. The method of embodiment 70 wherein the non-human primate is a Rhesus monkey, Cynomolgus monkey or baboon.

72. A method to evaluate the potency or efficacy of a test compound or test therapy to (i) increase the survival rate or probability of survival or (ii) enhance the survival experience of an exposed subject that has been exposed to a biological insult such as a radiation dose of about an LD_{50/30} or LD_{50/60}, comprising, (1) treating the exposed subject with an effective or potentially effective amount of the test compound or administering the test therapy, wherein the compound optionally has the structure; and (2) comparing the biological response of the test compound or test therapy in the exposed subject(s) with the same or a comparable biological response from a control exposed subject(s) of the same or a closely related species that has been treated with a F1C such as 3β,17β-dihydroxyandrost-5-ene or an analog thereof.

73. The method of embodiment 72 wherein the exposed subject is a human or a non-human primate.

74. The method of embodiment 58, 59, 60, 61, 62, 63, 64, 65, 66, 67, 68, 69, 70, 71, 72 or 73 wherein the increased survival rate or probability of survival or enhanced survival experience or biological response in the exposed subject(s) is (i) statistically significant at a level of P≦0.1, (ii) at least 5% increased or enhanced, (iii) at least 10% increased or enhanced, (iv) at least 15% increased or enhanced, (v) at least 20% increased or enhanced, (vi) at least 30% increased or enhanced or (vii) at least 40% increased or enhanced.

75. The method of embodiment 74 wherein the increased survival rate or probability of survival or enhanced survival experience or biological response in the exposed subject(s) is statistically significant at a level of P≦0.07, P≦0.05, P≦0.03 or P≦0.01.

76. A method to evaluate one or more biological characteristics of a test compound or therapy comprising evaluating the effect of the test compound or therapy on a surrogate marker for mortality in a human or a non-human primate that has been exposed to a biological insult such as a potentially lethal radiation dose that results in about an LD_{5/30} or LD_{5/60} or about an LD_{10/30} or LD_{10/60} or more, such as another radiation dose described herein.

77. The method of embodiment 76 wherein the surrogate marker for mortality is (i) duration of febrile severe neutropenia, (ii) duration of severe thrombocytopenia, (iii) duration of anemia, (iv) transient fever such as a spike to at least 40° C. in a Rhesus monkey within about 12-16 hours of the biological insult or (v) a biological response described at step (ii) of embodiment 14.

78. The method of embodiment 76 or 77 wherein the surrogate marker for mortality predicts death at a level of P≦0.10 or at a level of P≦0.05.

79. A method to (A) maintain approximately normal endogenous levels of 3β,17β-dihydroxyandrost-5-ene and/or 17β-hydroxyandrost-5-ene-3β-sufate in a human or non-human primate that has been or that is anticipated to be exposed to (i) a biological insult of at least about an LD₁₀, (ii) a cytotoxic chemotherapy or (iii) a radiation exposure or (B) decrease the drop in endogenous levels of 3β,17β-dihydroxyandrost-5-ene and/or 17β-hydroxyandrost-5-ene-3β-sufate in human or non-human primate, comprising, administering to the human or non-human primate an effective amount of a modulator compound selected from a steroid hydroxylase inhibitor, a 3-hydroxysteroid dehydrogenase inhibitor, a 17-hydroxysteroid dehydrogenase inhibitor, a steroid sulfotransferase inhibitor and an isoform or isozyme of any of these enzymes. Maintenance of approximately normal endogenous levels means that levels are maintained at least at about 30%, 40%, 50% or 60% of normal levels at times after a biological insult, e.g., at about 1, 2, 3 or 4 days to about 5, 10, 16, 18, 20 or 24 days after a significant radiation exposure or trauma. In humans normal levels of 3β,17β-dihydroxyandrost-5-ene are about 2-5 ng/mL and 17β-hydroxyandrost-5-ene-3β-sufate levels are about 350 ng/mL, although individual levels vary significantly.

80. The method of embodiment 79 wherein (a) the 17-hydroxysteroid dehydrogenase is a 17β-hydroxysteroid dehydrogenase optionally selected from type 1, 2, 3, 4, 5, 7, 8 or 10 human 17β-hydroxysteroid dehydrogenase or a non-human primate homolog or oxidative isoform of any of these enzymes; or (b) the 3-hydroxysteroid dehydrogenase is a 3β-hydroxysteroid dehydrogenase optionally selected from type 1, 2, 3, 4 or 5 human 3β-hydroxysteroid dehydrogenase or a non-human primate homolog or oxidative isoform of any of these enzymes; or (c) the 3-hydroxysteroid dehydrogenase is a 3α-hydroxysteroid dehydrogenase or a non-human primate homolog of any of a 3α-hydroxysteroid dehydrogenase; or (d) the sulfotransferase is a human sulfotransferase optionally selected from type 1 or 2 human sulfotransferase or a non-human primate homolog of any of these enzymes; or (e) the steroid hydroxylase is a 2-hydroxylase, a 7-hydroxylase, an 11-hydroxylase or a 15-hydroxylase, optionally selected from a human 2α-hydroxylase, a human 7α-hydroxylase, a human 6β-hydroxylase, a human 7β-hydroxylase, a human 15α-hydroxylase, an 11α-hydroxylase or an 11β-hydroxylase optionally selected from a human steroid CYP3A hydroxylase, a human steroid CYP2A hydroxylase, a human steroid CYP7B hydroxylase, a human steroid CYP2B hydroxylase or an isoform or homolog such as CYP3A1, CYP3A4, CYP3A4*1 B, CYP3A9, CYP3A23, CYP3A27, CYP3A45, CYP2A2, CYP1A2, CYP2A, CYP2A4, CYP2A5, CYP2A8, CYP2B, CYP2C, CYP2G1, CYP3A, CYP2A13, CYP2A5, CYP2A6 or a human or non-human primate homolog of any of these enzymes.

81. The method of embodiment 79 or 80 wherein the modulator compound has the structure, 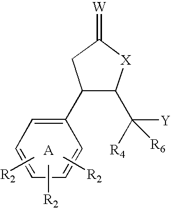
wherein the phenyl ring A and the —C(R₄)(R₆)Y group are oriented trans or cis to each other; W is ═O, ═S or ═NR,; X is —O—, —S—, —NH— or —NR₁—; R₁ is —H, —OH or —(C1-C4) alkyl optionally substituted with 1, 2, 3, or more halogen atoms; R₂ independently are —H, —F, —Cl, —Br, —I—N(R₃)₂, —OR₃, —SR₃, -phenyl optionally substituted with 1, 2, 3, 4 or 5 halogen atoms; or C1-C4 alkyl optionally substituted with 1, 2, 3 or more halogens or 1 or 2 independently selected —OR₃ or —SR₃; R₃ is —H or C1-C4 alkyl optionally substituted with up to three halogens; R₄ is —H or a bond terminating at the ortho position of phenyl ring A; Y is —F, —Cl, —Br —I, —OR₅, or —SR₅; R₅ is —H, C1-C4 alkyl optionally substituted with up to three halogens, phenyl optionally substituted with halogen, —OR_{3A}, —SR_{3A}, or —R_{3A}, or R₅ may independently be an optionally substituted phenyl group when R₆ is H, wherein there is 1, 2, 3, 4 or 5 halogen atoms, or one or two independently selected —OR_{3A}, —SR_{3A}, or —R_{3A} moieties; R_{3A} is —H or C1-C4 alkyl optionally substituted with up to three halogens; R₆ is (1) —H, optionally wherein when R₆ is —H, then Y is —OR₅ where R₅ is phenyl optionally substituted with halogen, —OR_{3A}, —SR_{3A}, or —R_{3A}. (2) a moiety having the structure 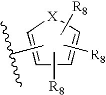
where R₈ independently are —H, optionally substituted alkyl, optionally substituted aryl, optionally substituted heterocycle, an ester, a thioester, or an R₁ moiety as defined for formula 1 compounds, optionally provided that when W is ═O and Y is —OH, 1, 2 or more of R₂ and R₈ are not —H and X is O or S; (3) —C1-C4 optionally substituted alkyl-aryl; (4) —CH═CH-optionally substituted aryl, —CH═CH-optionally substituted heteroaryl or —CH═CH-optionally substituted heterocycle; (5) —C≡C-optionally substituted aryl, —C≡C-optionally substituted heteroaryl or —C≡C-optionally substituted heterocycle; (6) —C≡C—CH₂-optionally substituted alkyl, —C≡C—CH₂-optionally substituted aryl, —C≡C—CH₂-optionally substituted heteroaryl or —C≡C—CH₂-optionally substituted heterocycle; (7) optionally substituted aryl, optionally substituted heteroaryl or optionally substituted heterocycle; (8) a moiety having the structure 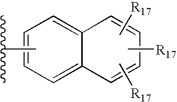
where R₁₇ independently are —H, optionally substituted alkyl, optionally substituted aryl, optionally substituted heterocycle, an ester, a thioester, or an R₁ moiety as defined for formula 1 compounds; or (9) the modulator is a compound selected from the group consisting of rel-(4R,5R)-5-[[5-(4-fluorophenyl)-2-thienyl](hydroxy)-methyl]-1-methyl-4-phenylpyrrolidin-2-one, rel-(4R,5R)-5-{(1S)-hydroxy[5-(3-pyridinyl)-2-thienyl]-methyl}-1-methyl-4-phenyl-2-pyrrolidinone, rel-5-((1R)-1-hydroxy-3-phenylprop-2-ynyl)(4R,5R)-1-methyl-4-phenylpyrrolidin-2-one, rel-(4R,5R)-5-[1H-indol-2-yl(methylthio)methyl]-1-methyl-4-phenylpyrrolidin-2-one, (4R,5R)-5-[(1R)-hydroxy(5-phenyl(2-thienyl))-methyl]-1-methyl-4-phenylpyrrolidin-2-one, rel-(4R,5R)-4-(4-aminophenyl-5-[hydroxy(5-phenyl-2-thienyl)methyl]-1-methylpyrrolidin-2-one, rel-(4R,5R)-4-(4-hydroxyphenyl)-5-[hydroxy(5-phenyl-2-thienyl)methyl]-1-methylpyrrolidin-2-one, rel-(4R,5R)-5-[(5-ethynyl-2-thienyl)(hydroxy)methyl]-1-methyl-4-phenylpyrrolidine-2-one, rel-(4R,5R)-5-{hydroxy[5-(1-oxidopyridin-3-yl)-2-thienyl]-methyl}-1-methyl-4-phenylpyrrolidin-2-one, rel-(4R,5R)-5-[2,2′-bithien-5-yl)hydroxy)methyl]-1-methyl-4-phenylpyrrolidin-2-one, rel-(4R,5R)-5-[[5-(2,6-difluorophenyl)-2-thienyl](hydroxy)-methyl]-1-methyl-4-phenylpyrrolidin-2-one, rel-(4R,5R)-5-{hydroxy[5-(3-methoxyphenyl)-2-thienyl]-methyl}-1-methyl-4-phenylpyrrolidin-2-one, rel-(4R,5R)-5-(hydroxy{5-[2-(trifluoromethyl)phenyl]-2-thienyl}methyl)-1-methyl-4-phenylpyrrolidin-2-one, rel-(4R,5R)-5-{hydroxy[5-(thiomorpholin-4-ylsulfonyl)-2-thienyl]methyl}-1-methyl-4-phenylpyrrolidin-2-one, rel-(4R,5R)-5-{hydroxy[5-(morpholin-4-ylsulfonyl)-2-thienyl]methyl}-1-methyl-4-phenylpyrrolidin-2-one, rel-(4R,5R)-5-[(5-{[4-(4-fluorophenyl)piperazin-1-yl]sulfonyl}-2-thienyl)(hydroxy)methyl]-1-methyl-4-phenylpyrrolidin-2-one, rel-(4R,5R)-5-[hydroxy(5-methyl-4-phenyl-2-thienyl)-methyl]-1-methyl-4-phenylpyrrolidin-2-one, rel-(4R,5R)-5-[hydroxy(5-methyl-3-phenyl-2-thienyl)-methyl]-1-methyl-4-phenylpyrrolidin-2-one, rel-(4R,5R)-5-{hydroxy[5-(3-nitrophenyl)-2-thienyl]-methyl}-1-methyl-4-phenylpyrrolidin-2-one, rel-(4R,5R)-5-[hydroxy(5-pyridin-2-yl-2-thienyl)methyl]-1-methyl-4-phenylpyrrolidin-2-one, rel-(4R,5R)-5-[[5-(4-chlorophenyl)-2-thienyl](hydroxy)-methyl]-1-methyl-4-phenylpyrrolidin-2-one, rel-(4R,5R)-5-{hydroxy[5-(4-hydroxyphenyl)-2-thienyl]-methyl}-1-methyl-4-phenylpyrrolidin-2-one, rel-5-[(5-fluoro-3-methyl-1-benzothien-2-yl)(hydroxy)-methyl]-1-methyl-4-phenylpyrrolidin-2-one, rel-(4R,5R)-5-[{5-[(E)-2-(4-fluorophenyl)vinyl]-2-thienyl}(hydroxy)methyl]-1-methyl-4-phenylpyrrolidin-2-one, rel-(4R,5R)-5-[{5-[2-(4-fluorophenyl)ethyl]-2-thienyl}-(hydroxy)methyl]-1-methyl-4-phenylpyrrolidin-2-one, rel-(4R,5R)-5-(hydroxy{5-[(4-methoxyphenyl)sulfonyl]-2-thienyl}methyl)-1-methyl-4-phenylpyrrolidin-2-one, rel-(4R, 5R)-5-[{5-[(4-chlorophenyl)sulfonyl]-2-thienyl}-(hydroxy)methyl]-1-methyl-4-phenylpyrrolidin-2-one, rel-(4R, 5R)-5-{hydroxy[5-(phenylthio)-2-thienyl]methyl}-1-methyl-4-phenylpyrrolidin-2-one, rel-(4R,5R)-5-[[5-(3-aminophenyl)-2-thienyl](hydroxy)-methyl]-1-methyl-4-phenylpyrrolidin-2-one, rel-4-(5-{hydroxy[(2R,3R)-1-methyl-5-oxo-3-phenylpyrro-lin-2-yl]methyl}-2-thienyl)benzoic acid, rel-(4R, 5R)-5-{hydroxy[5-(phenylsulfonyl)-2-thienyl]-methyl}-1-methyl-4-phenylpyrrolidin-2-one, rel-(4R, 5R)-5-[[5-(2-fluorophenyl)-2-thienyl]-(hydroxy)methyl]-1-methyl-4-phenylpyrrolidin-2-one, rel-(4R,5R)-5-[[5-(2-fluoro-4-methylphenyl)-2-thienyl]-(hydroxy)methyl]-1-methyl-4-phenylpyrrolidin-2-one, rel-N-[3-(5-{hydroxy[(2R,3R)-1-methyl-5-oxo-3-phenyl-pyrrolidin-2-yl]methyl}-2-thienyl)phenyl]-methanesulfonamide, rel-(4R,5R)-5-[{5-[3-(dimethylamino)phenyl]-2-thienyl}-(hydroxy)methyl]-1-methyl-4-phenylpyrrolidin-2-one, rel-(4R,5R)-5-[{5-[(4-fluorophenyl)sulfonyl]-2-thienyl}-(hydroxy)methyl]-1-methyl-4-phenylpyrrolidin-2-one, rel-(4R,5R)-5-[hydroxy(5-pyridin-3-yl-2-thienyl)methyl]-1-methyl-4-phenylpyrrolidin-2-one, rel-(4R,5R)-5-[hydroxy(5-pyridin-3-yl-2-thienyl)methyl]-1-methyl-4-phenylpyrrolidin-2-one hydrochloride, rel-4-(5-{hydroxy[(2R,3R)-1-methyl-5-oxo-3-phenylpyrrolidin-2-yl]methyl}-2-thienyl)benzonitrile, rel-(4R,5R)-4-(2-fluorophenyl)-5-[hydroxy(5-phenyl-2-thienyl)-methyl]-1-methylpyrrolidin-2-one, rel-(4R,5R)-4-(2-fluorophenyl)-5-[[5-(4-fluorophenyl)-2-thienyl](hydroxy)methyl]-1-methyl pyrrolidin-2-one, rel-(4R,5R)-4-(3-fluorophenyl)-5-[[5-(4-fluorophenyl)-2-thienyl](hydroxy)methyl]-1-methylpyrrolidin-2-one, rel-(4R,5R)-5-[[(4-fluorophenyl)-2-thienyl](hydroxy)-methyl]-4-(2-methoxyphenyl)-1-methylpyrrolidin-2-one, rel-(4R,5R)-4-(2-fluorophenyl)-5-{hydroxy[5-(phenyl-sulfonyl)-2-thienyl]methyl}-1-methylpyrrolidin-2-one, rel-(4R,5R)-5-[1-benzothien-2-yl(hydroxy)methyl]-4-(2-fluorophenyl)-1-methylpyrrolidin-2-one, rel-(4R,5R)-5-{hydroxy[5-(phenylsulfonyl)-2-thienyl]-methyl}-4-(2-methoxyphenyl)-1-methylpyrrolidin-2-one, rel-(4R,5R)-5-[{5-[(4-chlorophenyl)sulfonyl]-2-thienyl}-(hydroxy)methyl]-4-(2-methoxyphenyl)-1-methylpyrrolidin-2-one, rel (4R,5R)-5-[biphenyl-3-yl(hydroxy)methyl]-1-methyl-4-phenylpyrrolidin-2-one, rel-(4R,5R)-5-[(3,4-dichlorophenyl)(hydroxy)methyl]-1-methyl-4-phenylpyrrolidin-2-one, rel-(4R,5R)-5-[3-(4-fluorophenyl)-1-hydroxyprop-2-yn-1-yl]-1-methyl-4-phenylpyrrolidin-2-one, rel-(4R,5R)-5-[3-(2-chlorophenyl)-1-hydroxyprop-2-yn-1-yl]-1-methyl-4-phenylpyrrolidin-2-one, rel-(4R,5R)-5-[3-(2-fluorophenyl)-1-hydroxyprop-2-yn-1-yl]-1-methyl-4-phenylpyrrolidin-2-one, rel-(4R,5R)-5-{3-[3-(2,5-dimethyl-1H-pyrrol-1-yl)-phenyl]-1-hydroxyprop-2-yn-1-yl}-1-methyl-4-phenyl-pyrrolidin-2-one, rel-(4R,5R)-5-[3-(3-aminophenyl)-1-hydroxyprop-2-yn-1-yl]-1-methyl-4-phenylpyrrolidin-2-one, rel-(4R,5R)-5-[hydroxy(5-phenyl-2-thienyl)methyl]-1-methyl-4-phenylpyrrolidin-2-one, (4R,5R)-5-[[5-(4-fluorophenyl)-2-thienyl](hydroxy)-methyl]-1-methyl-4-phenylpyrrolidin-2-one, (4R,5R)-5-[hydroxy(5-pyridin-2-yl-2-thienyl)methyl]-1-methyl-4-phenylpyrrolidin-2-one, (4R,5R)-5-[hydroxy(5-pyridin-3-yl-2-thienyl)methyl]-1-methyl-4-phenylpyrrolidin-2-one, (4R,5R)-5-{hydroxy[5-(phenylsulfonyl)-2-thienyl]-methyl}-1-methyl-4-phenylpyrrolidin-2-one, rel-(4R,5R)-4-(2-fluorophenyl)-5-[hydroxy(2-thienyl)-methyl]-1-methylpyrrolidin-2-one, rel-(4R,5R)-4-(2-fluorophenyl)-5-hydroxy(5-pyridin-3-yl-3-thienyl)methyl]-1-methylpyrrolidin-2-one, rel-(4R, 5R)-4-(2-fluorophenyl)-5-[hydroxy(5-pyridin-3-yl-2-thienyl)methyl]-1-methylpyrrolidin-2-one hydrochloride, rel-(4R,5R)-4-(2-fluorophenyl)-5-[hydroxy)5-pyridin-2-yl-2-thienyl)methyl]-1-methylpyrrolidin-2-one, rel-(4R,5R)-5-[(3-chlorophenyl)(hydroxy)methyl]-4-(2-fluorophenyl )-1-methylpyrrolidin-2-one and rel-(4R,5R)-5-[2,2′-bithien-5-yl(hydroxy)methyl]-4-(2-fluorophenyl)-1-methylpyrrolidin-2-one.

82. The method of embodiment 79, 80 or 81 wherein the cytotoxic chemotherapy is a cancer chemotherapy.

83. The method of embodiment 79, 80 or 81 wherein the radiation exposure is exposure to at least about an LD₁₀ or at least about an LD₂₀.

84. The method of embodiment 83 wherein the radiation exposure is exposure to about an LD₄₀, about an LD₅₀, about an LD₆₀, about an LD₇₀, about an LD₈₀, about an LD₉₀, or about an LD₁₀₀.

85. The method of embodiment 79, 80, 81, 82, 83 or 84 wherein the level of 3β,17β-dihydroxyandrost-5-ene in the human's or non-human primate's blood, serum or plasma is maintained at a level of at least about 2 ng/mL, at least about 4 ng/mL or least about 10 ng/mL or at least about 12 ng/mL or at least about 15 ng/mL or least about 18 ng/mL or least about 20 ng/mL to at least about 25 ng/mL for a period of about 2, 3, 4, 5, 6, 7, 8, 9, 10, 11, 12, 13, 14, 15, 16, 17, 18, 19, 20 or 21 days after the biological insult, cytotoxic chemotherapy or radiation exposure, wherein the maintenance is for at least about 1 or 2 hours per day to at least about 4 or 12 hours per day.

86. The method of embodiment 79, 80, 81, 82, 83, 84 or 85 wherein an effective amount of 3β,17β-dihydroxyandrost-5-ene or another a formula 1 compound is administered to the subject, wherein the formula 1 compound is effective to treat, ameliorate or delay the onset of one or more blood cell deficiencies, optionally selected from the group consisting of severe or grade IV neutropenia, febrile severe or grade IV neutropenia and severe or grade IV thrombocytopenia.

87. The method of embodiment 86 wherein (a) about 5, 6 or 7 mg/kg/day to about 10, 12 or 15 mg/kg/day of 3β,17β-dihydroxyandrost-5-ene is administered to the human or non-human primate beginning at the about the time of the radiation exposure or beginning within about 1 hour, about 2 hours, about 3 hours, about 4 hours, about 5 hours, about 6 hours, about 8 hours, about 12 hours, about 24 hours, about 48 hours, about 72 hours or about 96 hours after the radiation exposure; and/or (b) the 3β,17β-dihydroxyandrost-5-ene is administered daily, every other day or every third day for a period of 1, 2, 3, 4, 5, 6, 7, 8, 9, 10, 11, 12, 13, 14, 15, 16, 17, 18, 19, 20 or 21 days after the radiation exposure.

88. The method of embodiment 86 or 87 wherein an effective amount of one, two or more treatments are administered to the subject on one, two, three, four or more occasions, wherein the one or more treatments optionally are (1) transfusion of one, two or more of blood, plasma, serum, red blood cells, white blood cells, cord blood cells, platelets or another blood product to the subject, (2) administration of one or more antimicrobial agents such as antibacterial agents or antifungal agents such as ciprofloxacin, a cephalosporin antibiotic, an amphotericin B such as amphotericin B lipid complex (ABLC) or liposomal amphotericin B, a triazole such as voriconazole, fluconazole or itraconazole or an echinocandin such as caspofungin, (3) administration of a chelating agent such as calcium diethylenetriaminepentaacetate (Zn-DTPA) (Ca-DTPA), zinc diethylenetriaminepentaacetate (Zn-DTPA), (4) administration of potassium 5 iodide, (5) administration of calcium disodium ethylenetriaminetetraacetic acid (CaNa₂EDTA), (6) a thymosin such as thymosin α-1 or thymosin β-4.

89. An F1C wherein the F1C contains or comprises one, two or more α,β-unsaturated ketone moieties.

90. The F1C of embodiment 89 wherein the α,β-unsaturated ketone moiety is 10 present at the A, B, C or D ring of the compound, or is present at a variable group that is bonded to the F1C.

91. The F1C of embodiment 89 or 90 wherein the F1C has the structure 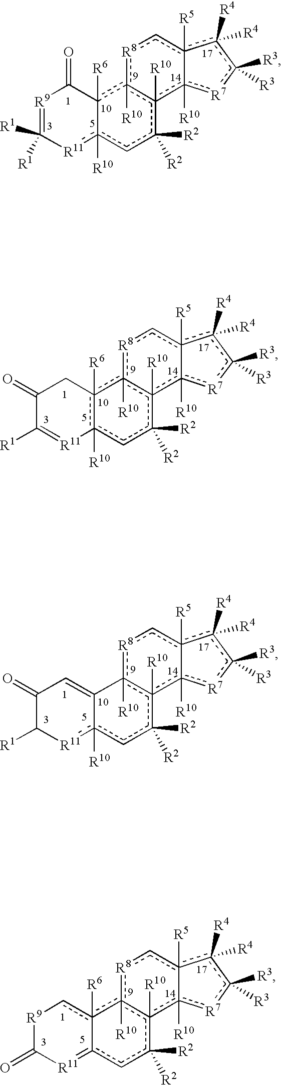 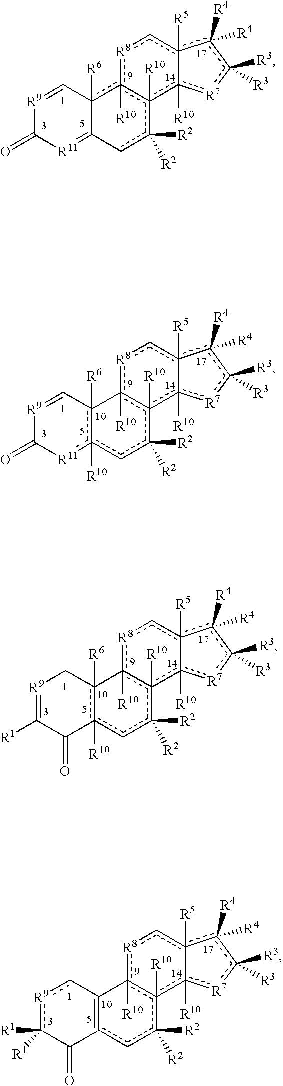 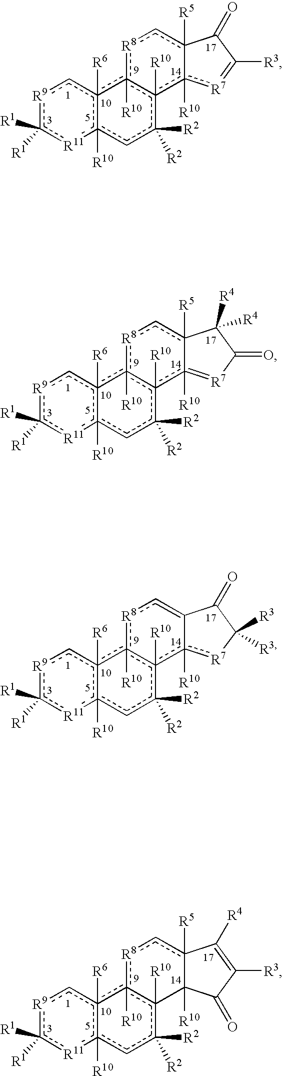 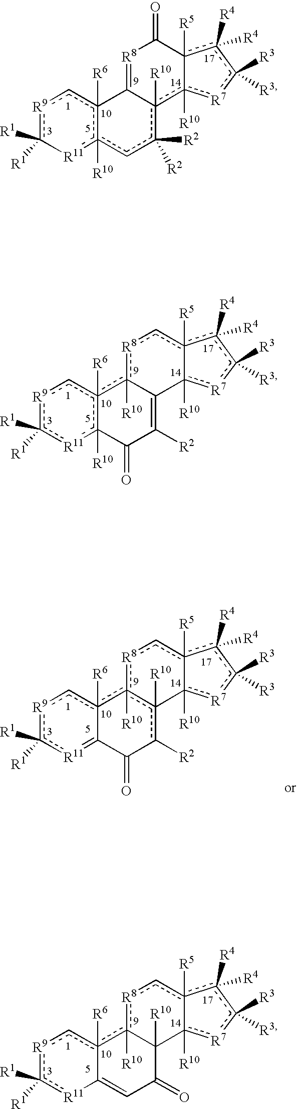
wherein 1, 2, 3 or 4 independently chosen R¹⁰ moieties at the 1-, 4-, 6- and 12-positions are optionally present and/or wherein the α,β-unsaturated ketone moiety at the variable group optionally has the structure —R^{X}—(CH₂)ₘ—CH═CH—C(O)—(CH₂)ₙ—CH₂—R^{Y} or —R^{X}—(CH₂)ₘ—C(O)—CH═CH—(CH₂)ₙ—CH₂—R^{Y}, where R^{X} is a single bond (absent), a double bond, —CH₂—, —CH(OH), —CH(CH₃), —CH(C₂H₅), —O—, —S—, —NH— or —N(optionally substituted alkyl), m is 0, 1, 2, 3, 4, 5 or 6, n is 0, 1, 2, 3, 4, 5 or 6 and R^{Y} is —H, —F, —Cl, —Br, —I, —OR^{PR}, —CH(O), —C(O)OR^{PR} or C1-C6 optionally substituted alkyl where R^{PR} is —H or a protecting group and where one or two methylene moieties in —(CH₂)ₙ— or —(CH₂)ₘ— are optionally independently substituted with an independently chosen substitution disclosed herein such as C1-C4 alkyl, C1-C4 substituted alkyl, C1-C4 alkylamine, —OH, —SH, —NH₂, —COOH, or halogen.

92. A method to identify a treatment method useful to increase the rate or probability of survival of an injured subject optionally selected from a human and a non-human primate, comprising, (a) exposing a subject such as non-human primates to a biological insult of at least about an LD_{40/30}, about an LD_{50/30}, about an LD_{60/30} or about an LD_{80/30} to obtain exposed subjects and conducting a treatment protocol obtain exposed treated subjects, wherein the exposed treated subjects are not provided with an ameliorative treatment selected from (i) a transfusion such as a whole blood transfusion(s), a platelet transfusion(s), or an immunoglobulin transfusion(s), (ii) an antimicrobial treatment(s) to treat or prevent an infection, (iii) assisted feeding such as feeding by parenteral or catheter feeding or by tube feeding to the stomach; and (b) determining the survival rate of the exposed treated subjects to obtain a treatment survival rate and comparing the treatment survival rate with a suitable control survival rate that was obtained from exposed subjects that were not provided with any treatment protocol and that were not provided with the ameliorative treatment.

93. The method of embodiment 92 wherein the biological insult is exposure of the subjects or the non-human primates to whole body radiation.

94. The method of embodiment 92 or 93 wherein the whole body radiation dose is about 600 cGy to about 635 cGy and the subject is a rodent or a non-human primate optionally selected from a rhesus monkey or a cynomolgus monkey. Radiation doses in these and other embodiments or methos described herein can optionally include doses of about 560 cGy, about 570 cGy, about 580 cGy, about 585 cGy, about 590 cGy, about 595 cGy, about 600 cGy, about 605 cGy, about 610 cGy, about 615 cGy, about 620 cGy, about 625 cGy, about 630 cGy, about 635 cGy, about 640 cGy, about 645 cGy, about 650 cGy, about 7 Gy, about 7.5 Gy, about 8 Gy, about 8.5 Gy, about 9 Gy, about 9.5 Gy, about 10 Gy, about 10.5 Gy and 11 Gy. Any of these doses can be administered as a single dose over a period of about 2 minutes to about 2 hours, or they can be administered in 1, 2, 3, 4, 5, 6, 7, 8, 10, 12 or more subdoses over a single day or over 2, 3, 4, 5, 6, 7, 10, 14, 21, 28 or more days or months.

95. The method of embodiment 92, 93 or 94 wherein the treatment protocol is administered beginning at about 21 days before through about 4 days after the exposure of the subjects or the non-human primates to the whole body radiation. In some embodiments, the treatment protocol is administered at a time beginning at about the same time of the exposure of the subjects or the non-human primates to the biological insult or beginning at about 15 minutes or about 30 minutes or about 1 hour to about 4 hours, about 6 hours, about 8 hours, about 10 hours, about 11 hours, about 12 hours, about 13 hours, about 16, about 24 hours, about 36 or about 48 hours after the biological insult exposure. Exemplary time ranges are at about 15 minutes or 1 hour after the biological insult exposure to about 8 hours, about 12 hours or about 24 hours after the biological insult exposure.

96. The method of embodiment 92, 93, 94 or 95 wherein the treatment protocol comprises administering about 0.1 mg/kg/day to about 60 mg/kg/day of a formula 1 compound. having the structure, optionally wherein the formula 1 compound is administered once per day daily or every other day for 1 to about 10 days, e.g., daily for 1, 2, 3, 4, 5, 6, 7, 8, 9 or 10 consecutive days.

97. The method of embodiment 92, 93, 94, 95 or 96 wherein the whole body radiation dose is about 590 cGy to about 650 cGy, e.g., about 600 cGy to about 635 cGy and the non-human primate optionally is a rhesus monkey or a cynomolgus monkey. In any of the methods or embodiments described herein where a subject is exposed to radiation, the radiation dose can optionally be calibrated prior to conduct of the method using two (2) acrylic phantoms placed in the same experimental set up that used for animal irradiation. Exposure time for each animal can be calculated based on circumference of each animal at the junction of the thorax and the abdomen. Actual dose received can be verified using One Dose (ONE DOSE®). Four dosimeters can be used for each animal. Dosimeters may be placed on the sagittal plane of the animal on the sternal, interscapular, lumbar and lower abdominal regions. Dosimetry measurements using phantom and One Dose dosimeters, the dose rate, duration of irradiation and the actual time of irradiation for each individual animal can be documented to control the radiation dose each animal receives. This allows calculation of radiation exposure time based on the thickness of each animal. Measurement of the circumference of non-human primates at the junction of the thorax and the abdomen for all animals can be used to provide dimensions for radiation exposure calculations. Phantoms of 2 or more different sizes can be used to estimate the exposure time for individual animals.

98. The method of embodiment 97 wherein the formula 1 compound has the structure 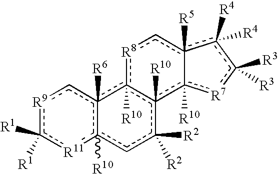
optionally wherein no double bond is present at the 17-position, R⁴ in the β-configuration is an O—, S— or N-linked moiety optionally selected from an ester, a thioether and a carmabate, R⁴ in the α-configuration is —H or a C-linked moiety optionally selected from optionally substituted alkyl and optionally substituted alkynyl, one R¹ is an O—, S— or N-linked moiety optionally selected from an ester, a thioether and a carmabate and the other R¹ is —H or a C-linked moiety optionally selected from optionally substituted alkyl and optionally substituted alkynyl or one R¹ is absent if a double bond is present at the 3-position and the remaining R¹ is an O—, S— or N-linked moiety, R² independently are —H or optionally substituted alkyl, R³ independently are —H, —F, —Cl, —Br, —I, optionally substituted alkyl or an O-linked moiety optionally selected from —OH, ═O and a carbonate, R⁵ is optionally substituted alkyl or is absent if a double bond is present at the 13-position, R⁶ is —H, optionally substituted alkyl or is absent if a double bond is present at the 13-position and R¹⁰ independently are —H, —F, —OH, optionally substituted alkyl or absent if a double bond is present at the ring carbon to which the R¹⁰ moiety is bonded.

99. A method to identify a treatment method useful to increase the rate or probability of survival of an injured subject such as a rodent, a human or a non-human primate, comprising; (1) exposing non-human primates to a biological insult of at least about an LD_{40/30} to obtain exposed subjects and conducting a treatment protocol obtain exposed treated subjects, wherein the exposed treated subjects are not provided with an ameliorative treatment selected from (i) a transfusion such as a whole blood transfusion(s), a platelet transfusion(s), or an immunoglobulin transfusion(s), (ii) an antimicrobial treatment(s) to treat or prevent an infection, (iii) assisted feeding such as feeding by parenteral or catheter feeding or by tube feeding to the stomach; and (2) determining the survival rate of the exposed treated subjects to obtain a treatment survival rate and comparing the treatment survival rate with a suitable control survival rate that was obtained from exposed subjects that were not provided with the ameliorative treatment, but that were treated with about 0.1 mg/kg/day to about 60 mg/kg/day or about 1 mg/kg/day to about 50 mg/kg/day about 2 mg/kg/day to about 45 mg/kg/day of a F1C optionally selected from androst-5-ene-3β,17β-diol, 17α-methylandrost-5-ene-3β,17β-diol, 17α-ethynylandrost-5-ene-3β,17β-diol, 16α-fluoroandrost-5-ene-3β,17β-diol, 16α-fluoroandrost-5-ene-3α,17β-diol, 16α-fluoroandrost-5-ene-3β,17α-diol, 16α-fluoroandrost-5-ene-3α,17α-diol, 16α-fluoro-17α-methylandrost-5-ene-3β,17β-diol, 16α-fluoro-17α-ethynylandrost-5-ene-3β,17β-diol, 16α-fluoroandrost-5-ene-17β-ol or 16α-fluoroandrost-5-ene-17α-ol or a 2-oxa, 4-ene, 5α-androstane, 5β-androstane and/or 19-nor analog of any of these compounds.

100. The method of embodiment 99 wherein the biological insult is exposure of the subjects or the non-human primates to whole body radiation, chemotherapy or a trauma such as one, two or more of hypoxia, hemorrhage, bone fracture, concussion or burn.

101. The method of embodiment 99 or 100 wherein the whole body radiation dose is about 600 cGy to about 635 cGy and the non-human primate is a rhesus monkey or a cynomolgus monkey.

102. The method of embodiment 99,100 or 101 wherein the treatment protocol is administered beginning at about 21 days before through about 4 days after the exposure of the subject or the non-human primates to the biological insult e.g., whole body radiation. In some embodiments, the treatment protocol is administered at a time beginning at about the same time of the exposure of the subjects or the non-human primates to the biological insult or beginning at about 15 minutes or about 30 minutes or about 1 hour to about 4 hours, about 6 hours, about 8 hours, about 10 hours, about 11 hours, about 12 hours, about 13 hours, about 16, about 24 hours, about 36 or about 48 hours after the biological insult exposure. Exemplary time ranges are at about 15 minutes or 1 hour after the biological insult exposure to about 8 hours, about 12 hours or about 24 hours after the biological insult exposure.

103. The method of embodiment 99, 100, 101 or 102 wherein the treatment protocol comprises administering about 0.1 mg/kg/day to about 60 mg/kg/day of a formula 1 compound, optionally wherein the formula 1 compound is administered once per day daily or every other day for 1 to about 10 days.

104. The method of embodiment 103 wherein the formula 1 compound has the structure 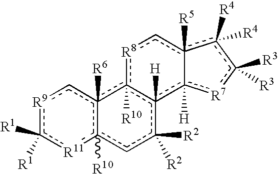
optionally wherein no double bond is present at the 17-position, R⁴ in the β-configuration is an O—, S— or N-linked moiety optionally selected from an ester, a thioether and a carmabate, R⁴ in the α-configuration is —H or a C-linked moiety optionally selected from optionally substituted alkyl and optionally substituted alkynyl, one R¹ is an O—, S— or N-linked moiety optionally selected from an ester, a thioether and a carmabate and the other R¹ is —H or a C-linked moiety optionally selected from optionally substituted alkyl and optionally substituted alkynyl or one R¹ is absent if a double bond is present at the 3-position and the remaining R¹ is an O—, S— or N-linked moiety, R² independently are —H or optionally substituted alkyl, R³ independently are —H, —F, —Cl, —Br, —I, optionally substituted alkyl or an O-linked moiety optionally selected from —OH, ═O and a carbonate, R⁵ is optionally substituted alkyl or is absent if a double bond is present at the 13-position, R⁶ is —H, optionally substituted alkyl or is absent if a double bond is present at the 13-position and R¹⁰ independently are —H or —F or absent if a double bond is present at the ring carbon to which the R¹⁰ moiety is bonded.

105. In the methods disclosed herein, the F1C can be administered at about 0.5 mg/kg/day to about 100 mg/kg/day or about 1 mg/kg/day to about 60 mg/kg/day or about 2 mg/kg/day to about 40 mg/kg/day. The F1C can be administered daily or intermittently, e.g., every other day or once per week. The F1C can also be administered daily for 2, 3, 4 or 5 consecutive days, followed by no administration for 1, 2, 3, 4, 5, 6, 7, 8 or more weeks, followed by again daily dosing for 2, 3, 4 or 5 consecutive days and this dosing cycle can be repeated 1, 2, 3 or more times. Typically the F1C is administered in a formulation that comprises 1, 2, 3, 4, 5 or more excipients. The F1C formulations can be solid or liquid formulations for parenteral, oral, sublingual, buccal, topical, aerosol, subcutaneous or subdermal administration. Unit doses, e.g., tablets or capsules can contain about 10 mg to about 1 g or about 1.5 g of the F1C, or about 20 mg or about 50 mg or about 100 mg to about 500 mg or about 750 mg.

106. A method comprising (a) administering a radiation dose of (i) about 580 cGy to about 640 cGy or (ii) about an LD_{30/30} to about an LD_{100/30}, to a non-human primate(s) and administering a test treatment compound or protocol to the irridiated non-human primate(s), optionally wherein the irridiated non-human primate(s) (or exposed treated subjects) are not provided with an ameliorative treatment selected from (i) a transfusion such as a whole blood transfusion(s), a platelet transfusion(s), or an immunoglobulin transfusion(s), (ii) an antimicrobial treatment(s) to treat or prevent an infection, (iii) assisted feeding such as feeding by parenteral or catheter feeding or by tube feeding to the stomach; and (b) optionally determining the survival rate of the exposed treated subjects to obtain a treatment survival rate and/or optionally comparing the treatment survival rate with a suitable control survival rate that was obtained from exposed subjects that were not provided with any treatment protocol and that were not provided with the ameliorative treatment.

107. The method of embodiment 106 wherein the radiation dose is whole body radiation, optionally wherein the dose is (i) about 600 cGy to about 635 cGy or (ii) about an LD_{35/30} to about an LD_{80/30}.

108. The method of embodiment 106 or 107 wherein the test treatment compound or protocol is administration of a F1C, optionally wherein the F1C is androst-5-ene-3β,17β-diol optionally wherein the F1C is a 1,5,9(11)-triene, 1,5,11-triene, 1,5,14-triene, 1,5,15-triene, 1,3,14-triene, 1,3,8(9)-triene, 1,3,8(14)-triene, 1,3,9(11)-triene, 1,3,11-triene or a 1,3,14-triene, optionally wherein one R¹ is an O-linked moiety or an N-linked moiety, the other R¹ is —H or a C-linked moiety, or, if a double bond is present at the 3-position, R¹ is O-linked moiety or an N-linked moiety, R⁴ in the β-configuration is an O-linked moiety, R⁴ in the α-configuration is —H or a C-linked moiety and one or both R³ independently or together are —H, —OH, ═O, an O-linked moiety, halogen or a C-linked moiety.

109. The method of embodiment 106, 107 or 108 wherein the survival rate of the exposed treated subjects is significantly increased compared to suitable control irridiated subjects of the same species or a closely related species or wherein the survival rate of the exposed treated subjects is increased by about 5%, about 10%, about 15%, about 20%, about 25%, about 30% or more compared to suitable control irridiated subjects of the same species or a closely related species or wherein the survival rate of the exposed treated subjects is increased with a significance of about p≦0.10, about p≦0.08 or about p≦0.05 or about p≦0.04.

110. The method of embodiment 106, 107, 108 or 109 wherein the F1C or the androst-5-ene-3β,17β-diol is administered once per day or once every 2^{nd} day or once every 3^{rd} day or once every 4^{th} day or once every 5^{th} day or once per week over a period of about 1, 2, 3, 4, 5, 6, 7, 8, 9, 10, 11, 12, 13, 14, 15, 16, 17, 18, 19, 20, 21, 22, 23 or 24 days beginning within about 0.5-12 hours after the irridiation of the irridiated non-human primate(s) or within about 1-6 hours or about 1-8 hours or about 2-4 hours or about 2-6 hours or about 1-10 hours after the irridiation of the irridiated non-human primate(s). In these embodiments, the test treatment compound or protocol can be administration of about 1 mg/kg/day or about 5 mg/kg/day to about 40 mg/kg/day or about 50 mg/kg/day of the F1C or of the 3β,17β-dihydroxyandrost-5-ene, e.g., about 2 mg/kg/day to about 20 mg/kg/day or about 4 mg/kg/day to about 20 mg/kg/day or about 5 mg/kg/day to about 15 mg/kg/day or about 5 mg/kg/day to about 30 mg/kg/day or about 10 mg/kg/day to about 30 mg/kg/day.

111. A method to treat or ameliorate a biological insult to a subject comprising administering to the subject an effective amount of a F1C, optionally wherein the effective amount of the F1C is determined by a method disclosed or claimed herein, any method of embodiment 106, 107, 108 or 109 or any of the original claims. The biological insult can be radiation exposure or exposure to a toxic chemotherapy or a poison.

For any of the methods, procedures or embodiments disclosed herein, when it is measured, survival can be determined at any convenient or useful time, e.g., at about 7, 10, 14, 15, 16, 17, 18, 21, 30, 60 or 180 days after the biological insult. For any of the methods, procedures or embodiments disclosed herein, the recited compound can be used for the preparation of a medicament to conduct or comprise the method, procedure or embodiment.

Variations and modifications of these embodiments, the claims and the remaining portions of this disclosure will be apparent to the skilled artisan after a reading thereof, e.g., portions on one disclosed embodiment or method can be combined with some or all of other embodiments, methods or portions of methods that are compatible therewith. Such variations and modifications are within the scope of this invention. All citations herein are incorporated herein by reference in their entirety. All citations herein are incorporated herein by reference with specificity. These citations are appended to this paragraph.

### EXAMPLES

The following examples further illustrate the invention and they are not intended to limit it in any way. Variations of these examples that are included in the invention may include, e.g., any of the F1Cs described herein or parts or all of any of the methods, formulations, treatment protocols and/or assays described herein.

### Example 1

Measurement of biological parameters in non-human primates after biological insult. A study was conducted to characterize a biological insult of 600 cGy of whole body irradiation to male Rhesus (Macaca mulatta) primates weighing 2.5 to 4.5 kg at an age range of about 1.75 to 3.5 years. Core body temperature was monitored by telemetry in the monkeys for a period of 40 consecutive days. Two groups of 10 animals each were used in the study. Core body temperature transmitters were surgically implanted in the abdomen prior to initiation of the radiation protocol. Core body temperature was continuously recorded from day -7 to day 41 for correlation with survival, hematology results, and other clinical parameters.

Temperature transmitters. Before initiation of the temperature transmitter implantation protocol, all animals were subject to a detailed physical examination and body weight measurement under the direction of a clinical veterinarian. Blood was collected from all animals, which were not food and water deprived, and assessed for basic blood chemistry and hematology. The results of the evaluation was reviewed by the clinical veterinarian to ensure satisfactory health status.

Implantation of the temperature transmitters was accomplished using animals that were fasted overnight prior to surgery and then anesthetized by an intra-muscular (IM) injection of acepromazine (10 mg/mL, 0.14 mg/kg) and ketamine (100 mg/mL, 13.6 mg/kg) and intubated. Where needed, lidocain spray (10% w/w) was administered onto the glottis prior to intubation. An ophthalmic ointment was applied to both eyes to prevent drying of the cornea. Animals were placed on a heating pad and administered isoflurane by inhalation, with an oxygen flow of approximately 200 mg/kg/min. A ventilator was used to maintain the respiratory rate between 8 and 20 breaths/min with a ventilation pressure of 18-25 cm H₂O. Monitoring during anesthesia included heart rate and oxygen saturation of the blood using a pulse oximeter. Prophylactic antibiotics (cefazolin 25 mg/kg) were administered by intramuscular injection at least 1-hour prior to surgery, and every 6 to 8 hours post injection for at least 24-hours post surgery. Analgesia (buprenorphine 0.05 mg/kg) was administered by intramuscular injection every 6 to 12 hours for at least 24-hours post surgery. Intravenous fluid therapy were given throughout the anesthesia using sterile Lactate Ringer's solution at a rate of 10 mL/kg/hr.

The surgical site was shaved and aseptically prepared using chlorhexidine gluconate 4% and isopropyl alcohol 70%. A longitudinal incision was performed lateral but close to the linea alba. The internal abdominal oblique muscle was separated from the aponeurosis of the transversus abdominis by blunt dissection. A sterile core body temperature transmitter (Data Science International, TA10TAD70) was inserted between the internal abdominal oblique muscle and the aponeurosis of the transversus abdominis. Hemostasis was maintained using appropriate suture material. Sterile saline was used to allow ease of placement of the transmitters. The incision was closed with absorbable suture material using simple continuous sutures. The skin was closed with discontinuous buried sutures using absorbable-suture material. Additional post-operative cares (analgesia and antibiotics) was provided to the animals when needed. Rectal body temperature was monitored in the post-operative period. Once the body temperature was within an acceptable range and the animal was alert, each animal was returned to their cages. A postoperative period of at least 2 weeks was allowed prior to initiation of radiation.

Acclimation and whole-body irradiation. Before transportation to the radiation facility, the animals were acclimated to the radiotherapy chair and to transportation. During the acclimation period, animals were assigned to their respective dose groups by block randomization based on the absolute neutrophil count. Any animal with unacceptable pretreatment data was replaced by an animal kept under identical environmental conditions. Animals with pretreatment data considered acceptable but marginally different from normal values were assigned to the sham group to allow longer post-operative recovery.

Animals were fasted overnight prior to whole-body irradiation and fed upon return to the holding facility. Animals were transferred to the irradiation facility in a transport vehicle with controlled environment. During transportation, each animal was individually housed in a stainless steel squeeze back cage. The animal's clinical signs were monitored immediately before and after transportation. Group 1 animals, sham irradiated, were subject to the same irradiation procedure as Group 2 animals, however, these animals did receive radiation. The 10 control animals, Group 1, were sham irradiated by placing each animal in the restraint for 10 minutes. The 10 treated animals, Group 2, received a midline ⁶⁰Co γ-radiation dose of 6 Gy at a dose rate of about 60 cGy/minute (day 1). The animals receiving this 6 Gy radiation insult were restrained during the radiation exposure by placing each animal in a chair allowing appropriate restraining in a symmetric position. An insulated cover was placed on the radiotherapy chair during transportation between the transport vehicle and the treatment site. Music was provided inside the treatment room to reduce stress to the animals. Animal positioning was confirmed with linear markers installed in the treatment room. To produce a homogenous dose distribution, treatment was divided in two parts. First, the animal received half of the dose by anteroposterior (AP) irradiation. The second half of the dose was delivered by posteroanterior (PA) irradiation. Group 1 animals were placed in an identical restraining chair in the sham treatment site for approximately the same period of time without exposure to radiation. Once the treatment was completed, animals were returned to the transport vehicle and were transported to their housing facility. The radiation dose was calibrated using an acrylic phantom placed in the same experimental set up that was used for animal irradiation.

Animal maintenance. Animals were housed individually in stainless steel squeeze back cages equipped with an automatic watering system except during transportation where water bottles were provided. The cages were labeled with a color-coded cage card indicating study number, group, animal number, species, sex and dose level. The animal room environment was controlled (temperature 21±3° C., humidity 30-70%, 10-15 air changes per hour, 12 hours light, 12 hours dark). Temperature and humidity were monitored continuously except during animal transportation and inside the radiation facility where only temperature was recorded. A standard certified commercial primate chow (Teklad Certified Global 25% Primate Diet # 2055C) was made available to each monkey daily. Food was withdrawn overnight prior to radiation and necropsy. Maximum allowable concentrations of contaminants in the diet (e.g., heavy metals, aflatoxin, organophosphates, chlorinated hydrocarbons and PCBs) were controlled and routinely analyzed by the manufacturers. If an animal stopped eating during the study, the diet was supplemented at the discretion of the study director. Tap water was purified by reverse osmosis and provided to the animals ad libitum throughout the study. Periodic analyses of the tap water and reverse osmosis water were performed. It was considered that there were no known contaminants in the diet or water. During the pre-treatment period cage side observations of clinical signs were generally performed once daily.

Observations. Mortality checks were performed twice a day during all phases of the study. Moribund animals were euthanized for humane reasons based on the clinical judgments. Sacrificed animals were subject to a clinical examination. When the core body temperature was 33° C. (91.4° F.) or lower or when an animal experienced a weight loss of more than 20% over a 4 day period, the animal was euthanized. Animals were also euthanized when they displayed complete anorexia for 3 days with deteriorating conditions based on the clinical examination or when they displayed an absence of response to stimuli.

Results obtained from the study were used to correlate the changes in biological parameters such as core body temperature and hematology with clinical signs following whole body irradiation. These results were used to obtain a status profile or surrogate endpoint such as incidence or duration of fever, followed by salvage with clinical support (antibiotics and blood transfusion), to assess the probability of survival or death of the treated individuals or similarly situated individuals that may have been subject to similar biological insults. During the pre-treatment period cage side observations of clinical signs were performed once daily. During the treatment period, clinical signs were recorded at cage-side twice daily for all animals or as often as deemed necessary. A detailed clinical examination was performed on all animals, once prior to irradiation on day 1, weekly thereafter, including on day 41 prior to necropsy.

The core body temperature and activity was recorded at 1 minute intervals for all animals from day −7 to day 41 using the implanted transmitter. Each animal cage was equipped with a telemetry receiver. The values of calibration of the transmitter implanted in each animal were entered in a telemetry computer system to ensure accurate temperature monitoring. Core body temperature was not recorded when animals were handled or during transport, but core temperature was generally monitored continuously at other times. Body weights were recorded for all animals once prior to randomization, prior to treatment on day 1 and weekly thereafter, including on day 40 (non-fasted) and on day 41 before necropsy. Hematology measurements were performed on all animals three times during the pre-treatment period and during the treatment period on days 2, daily from day 5 to day 27 and once on days 30, 33, 36 and 40. Blood samples of 0.5 mL were collected from the femoral vein or artery or from any appropriate vessel by venipuncture for hematological analysis. Food and water was available to the animals before blood collections.

Hematology parameters that were examined at most time points included red blood cell count, hematocrit, hemoglobin, white blood cell count, absolute differential WBC count, relative differential WBC count, relative reticulocyte count, mean corpuscular hemoglobin, platelet count, platelet volume, immature granulocyte count and red cell distribution width. EDTA was used as an anticoagulant and blood smears were prepared for each time point, stained with Modified Wright's stain and evaluated.

On day 41, the irradiated group 2 animals were sedated using ketamine and acepromazine and then euthanized by an overdose of barbiturate (e.g. sodium pentobarbital), which was administered intravenously, followed by exsanguination. For euthanized animals, gross pathology consisted of an external examination, identification of clinically recorded lesions and a detailed internal examination. To avoid autolytic changes, the necropsy examination was conducted as soon as possible on all animals that died while on study or that were euthanized during the study or at termination of the study at day 41. The animals were stored at 2-8° C. before examination. For all animals that were euthanized, the following organs were dissected, trimmed free of fat and weighed: Brain, testes, heart, prostate, kidneys, seminal vesicles, large intestine, small intestine, liver, spleen, lungs with trachea and thymus. The large intestine and small intestine were examined by making a longitudinal incision to open the lumen and removal of contents. The intestinal mucosa was washed with saline and excess saline was removed and the organs weighed. Paired organs were weighed together. Absolute and relative (to body weight) organ weights were calculated. On completion of the gross pathology examination, abnormal tissues brain (right part), femur and marrow, heart (both ventricles and atria, septum with papillary muscle), sternum and marrow, thymus were retained. Neutral buffered 10% formalin was usually used for fixation and preservation. Three femoral bone marrow smears were prepared from each euthanized animal (right femur), stained with Modified Wright's stain and evaluated.

Tissue samples from liver, lungs (right and left separately), kidneys, brain (left) and spleen were collected at necropsy from all euthanized animals for bacteriological culture. Tissue samples were stored refrigerated 2-8° C. pending analysis. A selected area at the surface of the tissue sample was burned to eliminate possible surface contaminant. A sterile culture swab was inserted in the tissue sample through the burned surface for isolation and identification of aerobic and anaerobic bacteria. Histopathological examination was performed on the tissues from euthanized animals. Tissues were prepared for histological examination by embedding in paraffin wax, sectioning and staining with hematoxylin and eosinphloxin.

### Example 2

Results and calculation of status profiles for non-human primates using biological parameter measurements. Numerical data obtained from the protocol described in example 1 was subjected to calculation of group means, standard deviations and other statistical analyses.

Statistically significant status profiles were obtained based on five biological parameters, i.e., anemia (based on hematocrit), thrombocytopenia (platelets), neutropenia (neutrophils), elevated temperature and circadian rhythm disruption. Each parameter alone gave statistically significant P_{lethality} and Pₛᵤᵣᵥᵢᵥₐₗ status profiles. When hematocrit nadirs for individual animals fell below 20% of normal, 4 of 4 animals died, while 5 of 6 animals survived when individual hematocrits remained above 20%. Calculation by an unpaired t-test analysis gave P_{lethality} and Pₛᵤᵣᵥᵢᵥₐₗ status profiles of 0.02 for a mean hematocrit nadir of 16.4% and 25.6% respectively.

When platelets for individual animals fell to less than 7,000 per μL, 5 of 6 animals died, while 4 of 4 animals survived when the platelet count nadir remained above about 7,000 per μL. Calculation by an unpaired t-test analysis of P_{lethality} and Pₛᵤᵣᵥᵢᵥₐₗ status profiles of 0.01 for a mean platelet nadir of 4,800 platelets per PL blood and 12,800 platelets per μL blood, respectively.

When the neutrophil nadir for individual animals fell to less than 50 per μL, 5 of 6 animals died, while 4 of 4 animals survived when the neutrophil count nadir remained above 50 per μL. Calculation by an unpaired t-test analysis of P_{lethality} and Pₛᵤᵣᵥᵢᵥₐₗ were 0.02 for a mean neutrophil nadir of 28 neutrophils per μL blood and 58 neutrophils per μL blood respectively.

For fever, P_{lethality} was less than 0.05 when the animals experienced fever or Pₛᵤᵣᵥᵢᵥₐₗ was greater than 0.95 when the animals did not have an elevated temperature or a fever. For this biological response, fever or elevated temperature was defined as a temperature of at least about 39.0° C. for at least about 15 minutes within 12 hours after the animals were irradiated on day 1. The baseline temperature for the animals was considered to be 37.3° C., although temperatures for the 10 control (non-irradiated) animals in example 1 varied with the animal's circadian rhythm between about 36.8° C. and 37.9° C. The control animal's circadian core body temperature rhythm was quite regular, while irradiated animals that survived the radiation was relatively regular and was indistinguishable from non-irradiated controls by about 5-8 days after irradiation. However, circadian core body temperature rhythm from irradiated animals that did not survive the radiation was destroyed and did not recover at any time after its disruption. P_{lethality} was less than 0.05 when circadian rhythm was disrupted, and Pₛᵤᵣᵥᵢᵥₐₗ was greater than about 0.95 when circadian rhythm was not disrupted. The loss of circadian rhythm was detectable within 24 to 48 hours after the animals were exposed to the 6 Gy dose of γ-radiation.

The P_{lethality} and Pₛᵤᵣᵥᵢᵥₐₗ status profiles for platelets, hematocrit and neutrophils given above was obtained using an unpaired T-test analysis based on the animals described in example 1. Five of the irradiated animals in example 1 survived the 6 Gy radiation exposure and the hematocrit, platelet and neutrophil nadir from irradiated surviving animals (variable 1) was compared to the hematocrit, platelet and neutrophil nadir from the 5 irradiated non-survivors (variable 2).

Hematocrit t-Test: Two-Sample Assuming Unequal Variances

| | variable 1 | variable 2 |
|---|---|---|
| Mean | 25.6 | 16.4 |
| Variance | 17.3 | 30.8 |
| Observations | 5 | 5 |
| Hypothesized Mean Difference | 0 | |
| df | 7 | |
| t Stat | 2.9662 | |
| P(T <= t) one-tail | 0.0105 | |
| t Critical one-tail | 1.8946 | |
| P(T <= t) two-tail | 0.0209 | |
| t Critical two-tail | 2.3646 | |

Platelet t-Test: Two-Sample Assuming Unequal Variances

| | variable 1 | variable 2 |
|---|---|---|
| Mean | 12.8 | 4.8 |
| Variance | 21.7 | 1.7 |
| Observations | 5 | 5 |
| Hypothesized Mean Difference | 0 | |
| df | 5 | |
| t Stat | 3.698001 | |
| P(T <= t) one-tail | 0.007014 | |
| t Critical one-tail | 2.015049 | |
| P(T <= t) two-tail | 0.014028 | |
| t Critical two-tail | 2.570578 | |

Neutrophil t-Test: Two-Sample Assuming Unequal Variances

| | variable 1 | variable 2 |
|---|---|---|
| Mean | 0.058 | 0.028 |
| Variance | 0.00037 | 7E−05 |
| Observations | 5 | 5 |
| Hypothesized Mean Difference | 0 | |
| df | 5 | |
| t Stat | 3.19801 | |
| P(T <= t) one-tail | 0.01202 | |
| t Critical one-tail | 2.01505 | |
| P(T <= t) two-tail | 0.02405 | |
| t Critical two-tail | 2.57058 | |

For the 5 surviving animals, the hematocrit nadirs were 28, 31, 24, 25 and 20, while hematocrit nadirs for the non-surviving animals were 14, 16, 12, 14 and 26. For the 5 surviving animals, the platelet nadirs were 10×10³ per μL, 18×10³ per μL, 12×10³ per μL, 17×10³ per μL and 7×10³ per μL, while platelet nadirs for the non-surviving animals were 5×10³ per μL, 4×10³ per μL, 4×10³ per μL, 4×10³ per μL and 7×10³ per μL. For the 5 surviving-animals, the neutrophil nadirs were 80 per mm³, 70 per mm³, 50 per mm³, 60 per mm³ and 30 per mm³, while neutrophil nadirs for the non-surviving animals were 20 per mm³, 30 per mm³, 20 per mm³, 40 per mm³ and 30 per mm³. The raw data for hematocrit, platelets and neutrophils from day −6 through day 26 are shown below and this data were used for the unpaired t-test P_{lethality} and Pₛᵤᵣᵥᵢᵥₐₗ calculations above.

Hematocrits (% or L/L) for irradiated animals at day −6 to day 10

| | day | | | | | | | |
|---|---|---|---|---|---|---|---|---|
| animal | −6 | 2 | 5 | 6 | 7 | 8 | 9 | 10 |
| 1 | 0.38 | 0.40 | 0.39 | 0.40 | 0.39 | 0.38 | 0.35 | 0.36 |
| 2 | 0.38 | 0.40 | 0.40 | 0.43 | 0.41 | 0.38 | 0.37 | 0.38 |
| 3 | 0.37 | 0.38 | 0.39 | 0.38 | 0.35 | 0.36 | 0.33 | 0.34 |
| 4 | 0.34 | 0.36 | 0.34 | 0.34 | 0.34 | 0.34 | 0.30 | 0.29 |
| 5 | 0.38 | 0.37 | 0.36 | 0.35 | 0.39 | 0.35 | 0.29 | 0.29 |
| 6 | 0.38 | 0.39 | 0.40 | 0.38 | 0.37 | 0.37 | 0.35 | 0.34 |
| 7 | 0.39 | 0.39 | 0.38 | 0.39 | 0.40 | 0.38 | 0.39 | 0.35 |
| 8 | 0.40 | 0.39 | 0.39 | 0.37 | 0.37 | 0.37 | 0.34 | 0.33 |
| 9 | 0.38 | 0.36 | 0.37 | 0.36 | 0.36 | 0.37 | 0.33 | 0.32 |
| 10 | 0.40 | 0.43 | 0.42 | 0.39 | 0.37 | 0.38 | 0.36 | 0.33 |
| mean | 0.38 | 0.39 | 0.38 | 0.38 | 0.38 | 0.37 | 0.34 | 0.33 |

Hematocrits (% or L/L) for irradiated animals at day 11 to day 18

| | day | | | | | | | |
|---|---|---|---|---|---|---|---|---|
| animal | 11 | 12 | 13 | 14 | 15 | 16 | 17 | 18 |
| 1 | 0.35 | 0.35 | 0.36 | 0.36 | 0.34 | 0.36 | 0.32 | 0.33 |
| 2 | 0.36 | 0.37 | 0.36 | 0.38 | 0.35 | 0.32 | 0.32 | 0.31 |
| 3 | 0.31 | 0.26 | 0.28 | 0.28 | 0.21 | 0.19 | 0.16 | 0.14 |
| 4 | 0.33 | 0.27 | 0.25 | 0.21 | 0.16 | * | | |
| 5 | 0.29 | 0.26 | 0.25 | 0.25 | 0.20 | 0.20 | 0.17 | 0.15 |
| 6 | 0.35 | 0.34 | 0.32 | 0.33 | 0.31 | 0.28 | 0.29 | 0.27 |
| 7 | 0.34 | 0.35 | 0.33 | 0.32 | 0.29 | 0.28 | 0.28 | 0.27 |
| 8 | 0.32 | 0.33 | 0.31 | 0.27 | 0.26 | 0.24 | 0.24 | 0.20 |
| 9 | 0.33 | 0.30 | 0.30 | 0.27 | 0.21 | 0.18 | 0.16 | 0.14 |
| 10 | 0.34 | 0.35 | 0.31 | 0.30 | 0.29 | 0.27 | 0.26 | 0.26 |
| mean | 0.33 | 0.32 | 0.31 | 0.30 | 0.26 | 0.26 | 0.24 | 0.23 |

* animal euthanized

Hematocrits (% or L/L) for irradiated animals at day 19 to day 26

| | day | | | | | | | |
|---|---|---|---|---|---|---|---|---|
| animal | 19 | 20 | 21 | 22 | 23 | 24 | 25 | 26 |
| 1 | 0.31 | 0.30 | 0.29 | 0.28 | 0.33 | 0.30 | 0.31 | 0.32 |
| 2 | 0.32 | 0.32 | 0.32 | 0.31 | 0.34 | 0.33 | 0.34 | 0.34 |
| 3 | * | | | | | | | |
| 4 | * | | | | | | | |
| 5 | ** | ** | 0.14 | 0.14 | 0.12 | ** | 0.12 | * |
| 6 | ** | 0.26 | 0.25 | 0.25 | 0.24 | 0.25 | 0.26 | 0.28 |
| 7 | 0.28 | 0.26 | 0.25 | 0.25 | 0.25 | 0.26 | 0.27 | 0.29 |
| 8 | 0.20 | 0.20 | 0.20 | 0.20 | 0.22 | 0.23 | 0.24 | 0.26 |
| 9 | * | | | | | | | |
| 10 | 0.29 | * | | | | | | |
| mean | 0.28 | 0.27 | 0.24 | 0.23 | 0.25 | 0.27 | 0.26 | 0.30 |

* animal euthanized
** measurement not obtained

Platelets (×10⁻³/μL) for irradiated animals at day −6 to day 10

| | day | | | | | | | |
|---|---|---|---|---|---|---|---|---|
| animal | −6 | 2 | 5 | 6 | 7 | 8 | 9 | 10 |
| 1 | 608 | 525 | 580 | 538 | 433 | 324 | 232 | 111 |
| 2 | 547 | 397 | 406 | 401 | 324 | 255 | 174 | 113 |
| 3 | 363 | 313 | 356 | 315 | 221 | 169 | 101 | 45 |
| 4 | 295 | 266 | 267 | 253 | 180 | 141 | 71 | 28 |
| 5 | 472 | 325 | 336 | 316 | 273 | 203 | 117 | 22 |
| 6 | 400 | 410 | 443 | 386 | 290 | 193 | 103 | 26 |
| 7 | 485 | 438 | 385 | 489 | 409 | 353 | 275 | 175 |
| 8 | 472 | 380 | 401 | 342 | 305 | 235 | 145 | 59 |
| 9 | 510 | 363 | 307 | 370 | 261 | 109 | 46 | 20 |
| 10 | 419 | 381 | 478 | 409 | 327 | 185 | 79 | 36 |
| mean | 457 | 380 | 396 | 382 | 302 | 217 | 134 | 64 |

Platelets (×10⁻³/μL) for irradiated animals at day 11 to day 18

| | day | | | | | | | |
|---|---|---|---|---|---|---|---|---|
| animal | 11 | 12 | 13 | 14 | 15 | 16 | 17 | 18 |
| 1 | 57 | 42 | 25 | 23 | 22 | 10 | 23 | 45 |
| 2 | 61 | 32 | 25 | 18 | 28 | 55 | 107 | 177 |
| 3 | 30 | 13 | 10 | 6 | 5 | 8 | 7 | 7 |
| 4 | 20 | 6 | 5 | 4 | 5 | * | | |
| 5 | 17 | 11 | 7 | 4 | 6 | 10 | 12 | 16 |
| 6 | 33 | 17 | 19 | 18 | 12 | 12 | 12 | 16 |
| 7 | 88 | 30 | 27 | 20 | 17 | 17 | 27 | 44 |
| 8 | 39 | 12 | 13 | 8 | 7 | 15 | 24 | 48 |
| 9 | 23 | 7 | 8 | 8 | 4 | 7 | 6 | 9 |
| 10 | 24 | 16 | 12 | 11 | 7 | 12 | 20 | 19 |
| mean | 39 | 19 | 15 | 12 | 11 | 16 | 26 | 42 |

* animal euthanized

Platelets (×10⁻³/μL) for irradiated animals at day 19 to day 26

| | day | | | | | | | |
|---|---|---|---|---|---|---|---|---|
| animal | 19 | 20 | 21 | 22 | 23 | 24 | 25 | 26 |
| 1 | 64 | 91 | 118 | 139 | 134 | 156 | 200 | 222 |
| 2 | 261 | 300 | 349 | 343 | 358 | 330 | 327 | 303 |
| 3 | * | | | | | | | |
| 4 | | | | | | | | |
| 5 | ** | ** | 44 | 53 | 74 | * | 186 | |
| 6 | ** | 44 | 104 | 142 | 217 | 259 | 305 | 318 |
| 7 | 89 | 144 | 278 | 353 | 448 | 523 | 519 | 514 |
| 8 | 90 | 92 | 158 | 194 | 246 | 285 | 341 | 406 |
| 9 | * | | | | | | | |
| 10 | 12 | * | | | | | | |
| mean | 103 | 134 | 175.17 | 217.00 | 246.17 | 310.60 | 313.00 | 352.60 |

* animal euthanized
** measurement not obtained

Neutrophils (×10⁻³/mm³) for irradiated animals at day −6 to day 10

| | day | | | | | | | |
|---|---|---|---|---|---|---|---|---|
| animal | −6 | 2 | 5 | 6 | 7 | 8 | 9 | 10 |
| 1 | 4.30 | 2.59 | 1.07 | 0.58 | 0.34 | 0.39 | 0.40 | 0.39 |
| 2 | 5.10 | 6.08 | 2.10 | 1.41 | 0.28 | 0.27 | 0.34 | 0.36 |
| 3 | 8.17 | 5.09 | 2.05 | 1.02 | 0.76 | 0.68 | 0.60 | 0.48 |
| 4 | 9.46 | 6.98 | 0.68 | 0.30 | 0.25 | 0.32 | 0.30 | 0.22 |
| 5 | 3.01 | 4.45 | 2.53 | 0.76 | 0.29 | 0.28 | 0.35 | 0.13 |
| 6 | 2.07 | 4.55 | 2.39 | 0.80 | 0.33 | 0.30 | 0.38 | 0.27 |
| 7 | 5.94 | 6.01 | 1.19 | 0.79 | 0.34 | 0.35 | 0.54 | 0.36 |
| 8 | 2.59 | 2.50 | 1.13 | 0.28 | 0.15 | 0.26 | 0.28 | 0.14 |
| 9 | 3.62 | 6.36 | 0.46 | 0.25 | 0.31 | 0.43 | 0.57 | 0.21 |
| 10 | 3.22 | 5.34 | 1.30 | 0.46 | 0.37 | 0.34 | 0.31 | 0.13 |
| mean | 4.75 | 5.00 | 1.49 | 0.67 | 0.34 | 0.36 | 0.41 | 0.27 |

Neutrophils (×10⁻³/mm³) for irradiated animals at day 11 to day 18

| | day | | | | | | | |
|---|---|---|---|---|---|---|---|---|
| animal | 11 | 12 | 13 | 14 | 15 | 16 | 17 | 18 |
| 1 | 0.17 | 0.10 | 0.08 | 0.14 | 0.17 | 0.09 | 0.10 | 0.08 |
| 2 | 0.30 | 0.15 | 0.10 | 0.10 | 0.07 | 0.07 | 0.19 | 0.46 |
| 3 | 0.13 | 0.09 | 0.12 | 0.09 | 0.04 | 0.05 | 0.07 | 0.02 |
| 4 | 0.16 | 0.11 | 0.04 | 0.06 | 0.03 | * | | |
| 5 | 0.07 | 0.09 | 0.06 | 0.07 | 0.02 | 0.04 | 0.10 | 0.24 |
| 6 | 0.13 | 0.10 | 0.13 | 0.09 | 0.06 | 0.06 | 0.05 | 0.05 |
| 7 | 0.24 | 0.19 | 0.10 | 0.06 | 0.12 | 0.20 | 0.12 | 0.15 |
| 8 | 0.11 | 0.06 | 0.05 | 0.05 | 0.03 | 0.05 | 0.18 | 0.69 |
| 9 | 0.13 | 0.16 | 0.11 | 0.06 | 0.08 | 0.05 | 0.06 | 0.04 |
| 10 | 0.09 | 0.09 | 0.05 | 0.07 | 0.03 | 0.04 | 0.07 | 0.05 |
| mean | 0.15 | 0.11 | 0.08 | 0.08 | 0.07 | 0.07 | 0.10 | 0.20 |

* animal euthanized

Neutrophils (×10⁻³/mm³) for irradiated animals at day 19 to day 26

| | day | | | | | | | |
|---|---|---|---|---|---|---|---|---|
| animal | 19 | 20 | 21 | 22 | 23 | 24 | 25 | 26 |
| 1 | 0.30 | 1.37 | 1.21 | 1.72 | 2.00 | 2.51 | 3.62 | 4.28 |
| 2 | 0.70 | 1.23 | 2.38 | 3.63 | 5.67 | 6.47 | 6.63 | 5.53 |
| 3 | * | | | | | | | |
| 4 | | | | | | | | |
| 5 | ** | ** | 2.57 | 4.51 | 4.60 | * | 6.04 | |
| 6 | ** | 0.18 | 0.30 | 1.57 | 1.32 | 2.12 | 5.52 | 6.14 |
| 7 | 0.14 | 0.08 | 0.27 | 0.83 | 1.66 | 3.38 | 5.54 | 11.53 |
| 8 | 1.80 | 0.84 | 1.86 | 4.07 | 2.82 | 3.6 | 3.59 | 6.77 |
| 9 | * | | | | | | | |
| 10 | 0.04 | * | | | | | | |
| mean | 0.60 | 0.74 | 1.43 | 2.92 | 3.01 | 3.62 | 5.16 | 6.85 |

* animal euthanized
** measurement not obtained

### Example 3

Treatment of whole body lethal radiation and characterization of mortality surrogate markers. Two groups of 10 Macaca mulatta (Rhesus monkey) were exposed to a 6 Gy dose of γ-radiation from a ⁶⁰Co source. This dose is an LD_{50/30} dose for this species. After irradiation, one group of animals was treated with test article, 15 mg/kg of 3β,17β-dihydroxyandrost-5-ene (“AED”) in vehicle, and the other 10-animal group was treated with the vehicle alone. The animals in each group were treated once per day for 5 consecutive days beginning on the day the animals were exposed to radiation. The animals consisted of 12 males and 8 females with a body weight range of about 2.5-5.5 kg at the onset of treatment. The age range was 1.75-5.0 years at the onset of treatment. Procedures involving the care and use of animals in this protocol was reviewed and approved by the Institutional Animal Care and Use Committee before conduct. During the study, the care and use of animals were conducted in accordance with the applicable rules and codes.

The animals were housed individually in stainless steel squeeze back cages equipped with an automatic watering system except during transportation where water bottles were provided. The cages were clearly labeled with a color-coded cage card indicating study number, group, animal number, species, sex and dose level. The animal room environment was controlled (temperature 21±3° C., humidity 30-70%, 10-15 air changes per hour, 12 hours light, 12 hours dark). Temperature and humidity was monitored continuously except during animal transportation and inside the radiation facility where only temperature was recorded. During transportation, only temperature was controlled. Air was not filtered during animal transportation and inside the radiation facility. A standard certified commercial primate chow (Teklad Certified Global 25% Primate Diet # 2055C) was made available to each monkey daily. Food was withdrawn overnight prior to radiation and necropsy. Maximum allowable concentrations of contaminants in the diet (e.g., heavy metals, aflatoxin, organophosphates, chlorinated hydrocarbons and PCBs) were controlled and routinely analyzed by the manufacturer. When an animal became inappetent during the study, the diet could be supplemented.

Tap water purified by reverse osmosis was provided to the animals ad libitum throughout the study. There were no known contaminants in the diet or water. Before transportation to the radiation facility, animals were acclimated to the radiotherapy chair and to transportation. Positive reinforcement was used to facilitate acclimation. Certified non human primate treats were given after acclimation periods. Twelve male and eight female rhesus monkeys were assigned to the study. Each group comprised of seven male and three female animals. During the acclimation period, animals were assigned to their respective dose groups by randomization based on the absolute neutrophil count. The average of 3 pretreatment absolute neutrophil counts were used for each animal.

The test article (100 mg/mL AED) and vehicle or control article in aliquots of 10 mL. Test article was an aqueous suspension in vehicle. The vehicle article consists of a solution of sodium shloride (0.9% w/v), carboxymethylcellulose (0.5% w/v), polysorbate 80 (2 % v/v), benzalkonium chloride (0.02 % v/v) and sodium phosphate (10 mM, pH 6.5). Immediately prior to drawing into a syringe, the test article formulation was briefly vortexed to uniformly distribute sedimented test article. Once drawn into a syringe, the test article was administered within 10 minutes. Just prior to injection, the syringe containing the test article was rotated end-over-end to uniformly disperse the compound.

During the pre-treatment period, body temperature transmitters were surgically implanted to allow core body temperature and physical activity monitoring. The animals were fasted overnight before the implant surgery. The animals were anesthetized by an intramuscular injection of acepromazine (10 mg/mL, 0.14 mg/kg) and ketamine (100 mg/mL, 13.6 mg/kg) and intubated. Where needed, lidocain spray (10% w/w) was administered onto the glottis prior to intubation. An ophthalmic ointment was applied to both eyes to prevent drying of the cornea. Animals were then placed on a heating pad and administered isoflurane by inhalation, with an oxygen flow of approximately 200 mL/kg/min or as needed. A ventilator was used to maintain the respiratory rate between 8 and 20 breaths/min with a ventilation pressure of 18-25 cm H₂O. Monitoring during anesthesia included heart rate and oxygen saturation of the blood using a pulse oximeter.

Prophylactic antibiotics (cefazolin 25 mg/kg) were administered by intramuscular injection at least 1-hour prior to surgery, and every 4 to 8 hours post injection for at least 24-hours post surgery. Analgesia (buprenorphine 0.05 mg/kg) was administered by intramuscular injection every 6 to 12 hours for at least 24-hours post surgery. Intravenous fluid therapy was given throughout the anesthesia using sterile Lactate Ringer's solution at a rate of 10 ml/kg/hr. The surgical site was shaved and was aseptically prepared using chlorhexidine gluconate 4% and isopropyl alcohol 70%. A longitudinal incision was performed lateral but close to the linea alba. The internal abdominal oblique muscle was separated from the aponeurosis of the transversus abdominis by blunt dissection. A sterile core body temperature transmitter (Data Science International, TA10TAD70) was inserted between the internal abdominal oblique muscle and the aponeurosis of the transversus abdominis. The serial number of the transmitter was recorded. Hemostasis was maintained using appropriate suture material. Sterile saline was used to allow ease of placement of the transmitters. The incision was closed with absorbable suture material using simple continuous sutures. The skin was closed with discontinuous buried sutures using absorbable suture material. Additional post-operative care (analgesia and antibiotics) were given to all animals where required. Rectal body temperature was monitored in the post-operative period. Once the body temperature was within an acceptable range and the animal was alert, each animal was returned to its cage. A post-operative period of at least 2 weeks was allowed prior to initiation of treatment. Core body temperature was monitored at 1 minute intervals beginning 6 days before radiation exposure and continued until 40 days after exposure.

Whole body radiation. The animals were exposed to ionizing as follows. Dosimetry measurements using phantoms, the dose rate and duration of irradiation and the actual time of irradiation for each individual animal was recorded. Animals were fasted overnight prior to whole-body irradiation and fed upon return to the housing facility. Animals were transferred to the treatment facility in a transport vehicle with controlled environment. During transportation, each animal was individually housed in a stainless steel squeeze back cage. Temperature in the transport vehicle was automatically recorded every 5 minutes during transportation. Clinical signs was monitored immediately before and after transportation.

Upon arrival to the site of irradiation, each animal was placed in a chair allowing appropriate restraining in a symmetric position. An insulated cover was placed on the radiotherapy chair during transportation between the truck and the treatment room. Each animal was brought in the treatment room. Music was provided inside the treatment room to reduce stress to the animals. Animal positioning was confirmed with linear markers installed in the treatment room.

The animals received a midline treatment dose of 600 cGy. The dose rate of the ⁶⁰Co gamma source was about 60 cGy per minute and the actual rate was recorded for each animal. To obtain a homogenous dose distribution, the radiation treatment was divided in two parts. First, the animal received half of the dose by anteroposterior irradiation. The second half of the dose was delivered by posteroanterior irradiation. Once the treatment was completed, animals were returned to the transport vehicle and transported to the housing facility. The radiation dose was calibrated using an acrylic phantom placed in the same experimental set up that was used for animal irradiation.

Administration of 3β,17β-dihydroxyandrost-5-ene and vehicle control. The animals received the vehicle control once daily for five (5) consecutive days by intramuscular injections. The first injection on day 1 was administered at 2-3 hours after irradiation. The dose volume was 0.15 mL/kg for all animals. The dose volume was evenly divided between two distinct sites (approximately 0.075 mL/kg per site). The actual volume delivered was calculated and adjusted based on each animal's body weight. To verify the concentration and homogeneity of the test and control articles in the dosing formulation, duplicate samples (1 mL/sample) from the bottom of each dosing formulation was taken prior to dosing on days 1, 2, 3, 4 and 5 and stored frozen (−70±10° C.) pending analysis.

During the pre-treatment period cage side observations of clinical signs were performed once daily. A detailed clinical examination was performed on all animals once prior to irradiation on day 1, day 9, weekly thereafter and at day 40 and 41. After radiation, the animals and clinical signs were observed twice a day during the protocol or as often as deemed necessary. Moribund animals were euthanized for humane reasons. Euthanasia criteria consisted of (i) a core body temperature of 35.9° C. after a period of febrile neutropenia, (ii) more than a 20% weight loss over a 3 day period, (iii) complete anorexia for 3 days with deteriorating conditions based on clinical examination or (iv) absence of response to stimuli.

Core body temperature and activity was recorded every minute for all animals from Day −10 to sacrifice using the implanted transmitter. Core body temperature and activity was recorded when animals were housed in their designated cage. Each designated cage was equipped with a telemetry receiver. Core body temperature was not recorded when animals were handled or during transport to the radiation facility. Body weights were recorded for all animals on the day following transfer, once before randomization, prior to treatment on day 1, day 9, weekly thereafter, at on the day the protocol ended. Laboratory hematology investigations were performed on all animals three times during the pre-treatment period and during the treatment period on day 2, daily from day 5 to day 27 and once on days 30, 33, 36 and 40.

For hematology analyses, blood samples of 0.5 mL were collected from the femoral vein or artery or from any appropriate vessel by venipuncture. EDTA was used as an anticoagulant. Animals were not deprived of food or water prior to blood collections. Parameters such as red blood cell count, hematocrit, hemoglobin, mean corpuscular volume, red blood cell count, mean corpuscular hemoglobin, white blood cell count, WBC differential (absolute), platelet count, WBC differential (relative), red cell distribution width, reticulocyte count and immature granulocyte count were measured. Blood smears were prepared for each timepoint, stained with Modified Wright's stain and evaluated.

For pharmacokinetic evaluation, blood samples (approximately 1.0 mL) were collected from all animals at about 22.0 to 23.5 hours following the first compound and control vehicle article administration on day 2. Each blood sample was collected into an EDTA potassium tube and kept on wet ice, for a maximum of 30 minutes, until centrifugation. The samples were centrifuged under refrigeration (2 to 8° C.) for approximately 10 minutes at 1500 g (RCF). The harvested plasma was transferred in one aliquot per sample. Blood samples (approximately 2.0 mL) was collected from all animals prior to sacrifice on days 40 and 41. Each blood sample was collected into an EDTA potassium tube and kept on wet ice, for a maximum of 30 minutes, until centrifugation. The samples were centrifuged under refrigeration (2 to 8° C.) for approximately 10 minutes at 1500 g (RCF). The harvested plasma was transferred in two separate aliquots per sample.

Liver, lung (right and left separately), kidney, brain (left) and spleen tissues were collected at necropsy from all euthanized animals for bacteriological culture. The tissue samples were stored refrigerated (2-10° C.) pending analysis. A selected area at the surface of the tissue sample was burned to eliminate possible surface contaminants. A sterile culture swab was inserted in the tissue sample through the burned surface for isolation and identification of aerobic and anaerobic bacteria

Numerical data obtained during the conduct of the study was subjected to calculation of group means and standard deviations. Data was analyzed using the Analysis of Variance (ANOVA) and the significance of inter-group differences were analyzed by Dunnett's “t” test or other appropriate tests using the SPSS for Windows, version 12.0, SPSS, Inc.

In the vehicle-treated control animal group, 4 of 10 animals survived, with 3 of the four non-survivors having febrile severe neutropenia, which was defined as a core body temperature of >40.4° C., i.e., ≧40.5° C., and an absolute neutrophil count of less than 500 cells/μL. In the 3β,17β-dihydroxyandrost-5-ene treated animal group, 9 of 10 animals survived, with the non-survivor not having febrile severe neutropenia and 2 survivors having the condition at some time during the protocol. Mortality in the untreated control group thus was 40% and 10% in the treated group. When the two control groups from this protocol and from the protocol described in examples 1 and 2 were combined, the total combined mortality of the 20 irridiated untreated animals was 45%. A reduction of mortality was observed (Fisher's exact test mid p=0.073) in the treated group compared to these two control groups. In the control group from this example only (vehicle treated) the animals experienced a median of 5 days of febrile severe neutropenia (95% CI 0,8) while the animals treated with 3β,17β-dihydroxyandrost-5-ene experienced a median of 0 days of febrile severe neutropenia (95% CI 0,2), giving a p=0.037 by the exact log rank test.

In the vehicle treated control animals from this example the animals collectively experienced 51 days of severe thrombocytopenia, less than 20,000 platelets/μL, while the animals treated with 3β,17β-dihydroxyandrost-5-ene collectively experienced 32 days of severe thrombocytopenia. This difference was p=0.009 by the exact test of homogeneity.

### Example 4

Non-human primate (NHP) lethal radiation studies. A meta analysis of 5 studies was conducted with a total of 100 animals. Four studies were conducted using 600 cGy (80 animals) and one study (20 animals) used 634 cGy of radiation. Before whole body radiation, Rhesus monkeys were acclimated to a lexan restraining device for all protocols and irradiated without the use of anesthesia or sedation. Animals were irradiated with either ⁶⁰Co photon or 6 MV X-ray radiation through anterior/posterior and posterior/anterior parallel-opposed ports (APPA) at a dose rate of approximately 60 cGy per minute to a total mid-plane dose of 6.00-6.34 Gy. Animals were given one half the dose AP and then rotated 180° at the mid-dose (3.00 Gy) to receive the remainder of the radiation. Simultaneous parallel-opposed ⁶⁰Co ports were used to deliver a mid-plane dose of 6.00 Gy at approximately 60 cGy per minute for animals in study 109.

For each study, experimental animals were randomly assigned to parallel groups, stratified by gender and body weight. All studies were vehicle controlled. A veterinarian managed animal care and data accrual. Baseline characteristics of the animals are shown below.

| | control | treated | all |
|---|---|---|---|
| Sample size | 50* | 50 | 100 |
| Sex M/F | 32/18 | 26/24 | 58/42 |
| Age (years) | | | |
| median | 3.38 | 3.69 | 3.59 |
| range | 2.3-6.32 | 2.08-5.18 | 2.08-6.32 |
| Body surface area** | | | |
| median | 2.39 | 2.46 | 2.44 |
| range | 2.03-3.12 | 1.89-3.08 | 1.89-3.12 |
| ANC/nL | | | |
| median | 4.07 | 3.75 | 3.86 |
| range | 1.52-11.09 | 1.23-8.14 | 1.23-11.09 |
| Platelets/nL | | | |
| median | 413.5 | 408.0 | 412.0 |
| range | 234-618 | 249-643 | 234-643 |
| Hgb(g/dL) | | | |
| median | 12.50 | 13.15 | 12.90 |
| range | 10.40-14.60 | 10.60-15.20 | 10.40-15.20 |
| Temperature (° C.) | | | |
| n | 61613 | 57324 | 118937 |
| median | 37.29 | 37.41 | 37.37 |
| range | 36.7-37.86 | 36.52-37.99 | 36.52-37.99 |

M/F: male/female ratio;
ANC: absolute neutrophil count;
Hbg: hemoglobin;
temperature was calculated from available dataon day -1.
*baseline body temperature was not collected for animals in the control group
**body surface area = cubed root of body weight squared

Between two to three weeks prior to irradiation, each animal underwent surgical implantation of a telemeter (model TA10TAD70, Data Sciences International) placed between the internal abdominal oblique muscle and the aponeurosis of the transverse abdominus muscle. This telemeter permitted remote, non-invasive monitoring of core body temperature by transmitting the data to a receiver once per minute. Core body temperature data were used to rapidly identify animals experiencing shock-related irreversible hypothermia, consequently permitting humane sacrifice. No clinical support consisting of transfusions or antibiotics was given to the animals other than buprenorphine analgesia if needed for pain control.

Each animal was observed twice daily (a.m. and p.m.) for mortality and evidence of pain/distress and findings were recorded as they were observed. Once daily, cage side assessments of food consumption and observations of each animal were performed. Strict euthanasia criteria to ensure humane sacrifice was developed and observed in all studies. The endpoints or criteria to humanely sacrifice animals included: (1) a 20% weight loss over a three-day period; (2) minimal to absent response to stimuli; (3) complete anorexia for 3 days with deteriorating conditions based on the clinical examination; (4) a drop in core body temperature to 36° C. (96.8° F.) or lower following a febrile episode with a temperature of 40° C.; and (5) severe acute anemia of 4 gm Hgb and/or <13% hematocrit.

The animals were treated with a parenteral formulation containing androst-5-ene-3β,17β-diol, which was administered intramuscularly, based on body weight (mg/kg), 3-4 hours after irradiation and continued once daily for a total of five injections. Individual animal doses were based on the most recently recorded body weight prior to total body radiation (TBI). Doses used in the studies were 5,10 and 15 mg/kg/day. The animals were subjected to blood collection (0.5 mL) as follows: 3 times during the pretreatment period and on study days 2, 5 through 27, 30, 33, 36 and 40. Blood counts were measured using an automated hematology analyzer (Advia 120) and the absolute differential leukocyte counts, hemoglobin and hematocrit were estimated.

Laboratory data were imported into an electronic database (SAS Institute Inc. 2004, SAS OnlineDoc v9.1. Cary, N.C.: SAS Institute Inc.) Specific rules for imputation of missing hematological data followed regulatory guidance. The duration of a given cytopenia was expressed relative to the number days at risk. Studies were designed to explore early activity and hence were not powered to formally detect efficacy. Statistical analyses were performed with the use of the SAS System and StatXact software for exact p-values. All reported p values were based on two-sided tests without adjustment for multiple comparisons. The cumulative incidence of cytopenia explores profile differences in the pattern of accumulation. Analysis by nonparametric mean cumulative function models were tested according to both Nelson's approach and semi-parametric regression models for repeated events. Mortality data was analyzed by means of the exact Fisher's test. Prediction was implemented by generalized linear mixed models. 100 non-human primates were lethally irradiated in five studies conducted at two laboratories under GLP guidelines. All animals were clinically healthy at entry. The probability of natural death was expected to be near zero i.e., if it were not for the irradiation event, all animals were expected to survive the 36-40 day duration of the various experiments. Protocol inclusion and exclusion criteria tended to promote a uniform selection of individuals. A total of 32 monkeys (32%) died in the course of the studies. Of these, 24 (75%) were the result of euthanasia in extremis. One study, performed at 634 cGy, was included in the total. The treatment difference in mortality between treatments is 20 percentage points at each radiation level. The point estimate of mortality in controls at 600 cGy is 32.5% (13/40; 95% CI: 19.34% to 48.32%). The overall odds ratios suggest that benefit may be substantial, although not quite significant for the present sample size. Mortality events took place in a rather narrow time band. All deaths occurred in 13 days, between Study Day 14 and 26. By Study Day 18, 50% of the deaths had already occurred. Only 4/32 individuals (12.5%) died after Study Day 20. These late deaths do not seem to represent any particular study or trend to delay euthanasia. Individuals in study 2973 (634 cGy) experienced 70% overall mortality. Deaths in this study occurred sooner than in the other studies (medians: 17 days in 2973 vs 19 days in the other studies; p=0.04. Only 1 of the 11 animals that died in the compound treated group (90.9%) was attributable to hemorrhage, whereas 14 of the 21 deaths in the vehicle group were ascribed to hemorrhage. This difference was highly significant (p=0.0028). A summary is shown below.

| | | | drug | deaths | deaths | odds |
|---|---|---|---|---|---|---|
| Study | | radiation | dose | n/N | n/N | ratio |
| number | n | cGy | (mg/kg) | treated | control | (Cl) |
| 1 | 10 | 600 | NA | NA | 5/10 | — |
| 2 | 20 | 600 | 15 | 1/10 | 4/10 | 0.17 |
| | | | | | | 0.00, 2.45 |
| 3 | 20 | 600 | 5 | 1/10 | 2/10 | 0.44 |
| | | | | | | 0.01, 10.5 |
| 4 | 30 | 600 | 5 | 2/10 | 2/10 | 0.71 |
| | | | 10 | 1/10 | | 0.07, 10.15 |
| all 600 | 80 | 600 | 5-15 | 5/40 | 13/40 | 0.39* |
| cGy | | | | | | 0.11, 1.39 |
| 5 | 20 | 634 | 15 | 6/10 | 8/10 | 0.37 |
| | | | | | | 0.03, 3.89 |
| total for | 100 | — | — | 11/50** | 21/50 | 0.39* |
| all | | | | | | 0.13, 1.13 |

NA: study did not include a drug treatment arm.
*common odds ratio stratified by study
**mid-p value = 0.044

As shown above, for the 100 animals, there were 32 total deaths, 21 in untreated animals and 11 in the treated animals. The primary cause of death by infection was observed for 7 animals in the placebo groups (33%) and 10 (90.9%) in the treated groups, but when the primary cause of death was hemorrhage 14 (66.7%) placebo animals died from hemorrhage, while only 1 (3.1%) treated animal died from hemorrhage.

Lower platelets counts at baseline correlated with a greater propensity to die of acute radiation syndrome (ARS). The proximal link involved some form of transfer to platelet count on day 2. Predictions range from 12.42%, for an observed platelet count of 643/vL (nanoliter), to 52.51 % for a baseline count of 234/vL. No other baseline variable related to post irradiation survival. Treatment with the compound given on day one was predictive of survival. Fever greater than 39° C. at day 3 predicts survival (p=0.006) (R²=0.11; ΔP(IQR)=−0.28). Furthermore, the effect of androst-5-ene-3β,17β-diol treatment is already evident by then with a 9 to 17% decrease in the chances of death for the treated individuals (p<0.013). However excess deaths were noted for animals with fever below 39° C.

Accumulation of days of thrombocytopenia [Sₜ] up to day 14 (before the first death) accurately predicts mortality when coupled with treatment (87/100; R²=0.62; p<0.001). In addition, androst-5-ene-3β,17β-diol treatment and a higher platelet count at Day 14 each predicts increased survival. A greater accumulation of days of thrombocytopenia (<20,000 platelets/μL) up to Day 14 was associated with decreased survival. The accumulation of days severe neutropenia (Sₙ) plays no major predictive role at day 14. Febrile severe neutropenia was infrequent, 6.13% and 5.43% of the days at risk in the control and androst-5-ene-3β,17β-diol treated groups respectively (p>0.44). Results at day 26 (last death) can be explained in terms of the model proposed for day 14, with Sₙ extended up to day 26 to account for the fate of 92/100 individuals. The duration of severe neutropenias and severe thrombocytopenias is shown below. For the data shown below, the following meanings apply. IQR=inter-quartile limits; * p<0.05, ** p<0.01, *** p<0.001; ANC<0.5 means less than 0.5 neutrophils/nL and Plat<50 means less than 50 platelets/nL.

| | | | To Day 14 (%) | | To Day 26 (%) | |
|---|---|---|---|---|---|---|
| Study | Parameter | | control | treated | control | treated |
| 1 | ANC < 0.5 | Median | 72.73 | | 63.045 | |
| | | IQR | (72.73, 81.82) | | (56.52, 83.33) | |
| | Plat < 20 | Median | 27.27 | | 31.88 | |
| | | IQR | (9.09, 27.27) | | (8.7, 43.75) | |
| 2 | ANC < 0.5 | Median | 72.73 | 63.64** | 73.91 | 56.52*** |
| | | IQR | (72.73, 72.73) | (54.55, 72.73) | (65.22, 80.0) | (43.48, 60.87) |
| | Plat < 20 | Median | 13.635 | 9.09 | 28.26 | 13.045 |
| | | IQR | (0, 18.18) | (9.09, 18.18) | (0, 50.0) | (4.35, 17.39) |
| 3 | ANC < 0.5 | Median | 59.09 | 63.64 | 50.0 | 52.17 |
| | | IQR | (54.55, 72.73) | (54.55, 72.73) | (39.13, 65.22) | (47.83, 60.87) |
| | Plat < 20 | Median | 13.635 | 9.09 | 17.39 | 15.215 |
| | | IQR | (9.09, 18.18) | (0, 18.18) | (13.04, 30.43) | (8.7, 21.74) |
| 4 | ANC < 0.5 | Median | 72.73 | 72.73* | 60.87 | 58.26 |
| | | IQR | (72.73, 81.82) | (63.64, 72.73) | (56.52, 78.26) | (50, 63.38) |
| | Plat < 20 | Median | 18.18 | 13.635 | 21.74 | 13.04 |
| | | IQR | (0, 18.18) | (0, 22.72) | (13.04, 21.74) | (2.175, 19.56) |
| All | ANC < 0.5 | Median | 72.73 | 72.73* | 63.045 | 56.52** |
| 600 | | IQR | (72.73, 72.73) | (54.55, 72.73) | (56.52, 78.26) | (47.83, 60.87) |
| cGy | Plat < 20 | Median | 18.18 | 9.09 | 21.74 | 13.04** |
| | | IQR | (9.09, 27.27) | (0, 18.18) | (13.04, 40.05) | (4.35, 21.74) |
| 5 | ANC < 0.5 | Median | 72.73 | 77.27 | 73.86 | 72.73 |
| (634 | | IQR | (72.73, 81.82) | (72.73, 81.82) | (71.43, 80.0) | (60.87, 78.57) |
| cGy) | Plat < 20 | Median | 27.27 | 22.72 | 35.24 | 24.50 |
| | | IQR | (18.18, 27.27) | (18.18, 27.27) | (31.25, 41.67) | (17.39, 36.36) |

The results demonstrated that androst-5-ene-3β,17β-diol administered intramuscularly 2-4 hours after lethal TBI and daily for total of five days significantly reduced the number of deaths in treated monkeys by 62.5% (LD₁₂ in treated groups vs. LD₃₂ in control groups). This is the first demonstration of a drug treatment resulting a reduction in deaths in lethally irradiated, clinically unsupported non-human primates. Previous studies have used cytokines alone or in combination in lethally irradiated NHP to study the kinetics of bone marrow recovery from the ensuing radiation-induced hematopoietic syndrome. These studies were conducted in NHP given full clinical support with antibiotic regimens and transfusions for thrombocytopenia and anemia following TBI.

Administering androst-5-ene-3β,17η-diol enhanced recovery of both platelets and neutrophils in radiation-induced myelosuppression of bone marrow. This suggests that androst-5-ene-3β,17β-diol increases the numbers or activity of early progenitor stem cells, if not the stem cell itself. Studies are underway to identify the target cell population and the mechanism of action. Although androst-5-ene-3β,17β-diol enhanced the recovery of neutrophils, platelets and red blood cells, its impact was most readily apparent on platelets. The duration of grade 4 thrombocytopenia (<20,000 platelets/μL) appeared to correlate with death to a greater extent than the duration of grade 4 neutropenia. The data are consistent with the hypothesis that both febrile grade 4 neutropenia and grade 4 thrombocytopenia contribute to the mortality of lethally irradiated subjects. In one lethal study with untreated animals, the temporal relationship between the time of death and bleeding episodes suggested that hemorrhage was a precipitating factor that lead to death in the majority of animals. Consequently, thrombocytopenia becomes more clinically relevant than previously appreciated. This finding is somewhat surprising, since the role of neutrophils to prevent infection and presumably sepsis leading to death in myelosuppressed patients has been established in the medical literature. (Bodey et al, Ann. Intern. Med. 64(2):328-340 1966; Crawford et al, New Engl. J. Med. 325(3): 164-170 1991) It is reasonable to expect that the most important factor in preventing death in this NHP model would be protection and restoration of neutrophils to fight infection. This is likely true where bone marrow suppression is not complete as is the case in marrow suppression commonly seen in chemotherapy-induced myelosuppression in the clinic. In this population adequate stem cells remain in the marrow and can be recruited by various cytokines such as G-CSF, GM-CSF, TPO, Flt-3 ligand to differentiate into mature elements of the marrow, especially neutrophils. Where there is a more profound effect on stem cells, the data suggest that the most important factor correlated with death is the duration of severe thrombocytopenia. Up to day 14, when the first death occurred on study, absolute neutrophil counts and duration of febrile (>40.4 ° C.) severe neutropenia (ANC<500 cells/μL) were not correlated with death. Although febrile grade 4 neutropenia is a consequence of radiation exposure, it is one of only several factors contributing to mortality. In these studies, grade 4 thrombocytopenia associated with hemorrhage appeared to be the primary cause of death in the majority of untreated primates. An early recovery of thrombopoeisis stimulated by androst-5-ene-3β,17β-diol minimizes a subject's time at risk for hemorrhage. This shorter duration of grade 4 thrombocytopenia translates into improved survival. The pathophysiology associated with this phenomena may be small areas of hemorrhage including petechiae in the aerodigestive tract providing an easy and early entry point of opportunistic bacteria and subsequent prolongation of neutropenia allowing microbial growth unchecked by the immune system and eventually contribute to the observed mortality. Consistent with this, enhanced recovery of both platelets and neutrophils have more a positve effect on mortality than on recovery of neutrophils alone.

To the extent not already indicated, it will be understood by those of ordinary skill in the art that any of the various specific embodiments, analysis methods, compounds or compositions described herein may be modified to incorporate other appropriate features, e.g., as shown in any other of the specific embodiments disclosed herein or in any of the methods described in the cited references.

What is claimed is:

1. A method to identify a treatment method useful to increase the rate or probability of survival of an injured human or non-human primate, comprising
(a) exposing non-human primates to a biological insult of at least about an LD_{30/30} to obtain exposed subjects and conducting a treatment protocol obtain exposed treated subjects, wherein the exposed treated subjects are not provided with an ameliorative treatment selected from (i) a transfusion such as a whole blood transfusion(s), a platelet transfusion(s), or an immunoglobulin transfusion(s), (ii) an antimicrobial treatment(s) to treat or prevent an infection, (iii) assisted feeding such as feeding by parenteral or catheter feeding or by tube feeding to the stomach; and
(b) determining the survival rate of the exposed treated subjects to obtain a treatment survival rate and comparing the treatment survival rate with a suitable control survival rate that was obtained from exposed subjects that were not provided with any treatment protocol and that were not provided with the ameliorative treatment.

2. The method of claim 1 wherein the biological insult is exposure of the non-human primates to whole body radiation.

3. The method of claim 2 wherein the whole body radiation dose is about 600 cGy to about 635 cGy and the non-human primate is a rhesus monkey.

4. The method of claim 2 wherein the treatment protocol is administered beginning at about 21 days before through about 4 days after the exposure of the non-human primates to the whole body radiation.

5. The method of claim 2 wherein the treatment protocol comprises administering about 0.1 mg/kg/day to about 60 mg/kg/day of a formula 1 compound having the structure 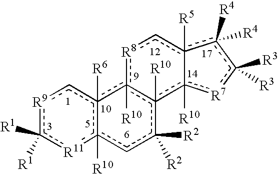
wherein the dotted lines are optional double bonds and 0 1, 2, 3, 4 or 5 double bonds are present;
each R¹, R², R³, R⁴, R⁵, R⁶ and R¹⁰ independently or together are —H, —OH, —OR^{PR}, —SR^{PR}, —SH, —N(R^{PR})₂, —NHR^{PR}, —NH₂, —O—Si—(R¹³)₃, —CHO, —CHS, —CN, —SCN, —NO₂, —N₃, —COOH, —COOR^{PR}, —OSO₃H, —OSO₂H, —OPO₃H₂, ═O, ═S, ═N—OH, ═N—OCH₃, ═CH₂, ═CH—CH₃, ═CH-optionally substituted alkyl, ═N-optionally substituted alkyl, ═N—O-optionally substituted alkyl, —NH—S(O)(O)-optionally substituted alkyl, —S—S-optionally substituted alkyl, ester, thioester, thionoester, phosphoester, phosphothioester, phosphonate, phosphonate ester, thiophosphonate, thiophosphonate ester, phosphiniester, sulfite ester, sulfate ester, sulfamate, sulfonate, sulfonamide, amide, amino acid, peptide, ether, thioether, acyl, thioacyl, carbonate, carbamate, halogen, optionally substituted alkyl, optionally substituted alkenyl, optionally substituted alkynyl, optionally substituted aryl, optionally substituted heteroaryl, optionally substituted heterocycle, optionally substituted monosaccharide, optionally substituted oligosaccharide, polymer, spiro ring, epoxide, acetal, thioacetal, ketal or a thioketal, ═N—O-optionally substituted alkyl, ═N-optionally substituted alkyl, —NH-optionally substituted alkyl, —N(optionally substituted alkyl)₂ where each optionally substituted alkyl is independently selected, or, one or more of two adjacent R¹, R², R³, R⁴, R⁵, R⁶ and R¹⁰ comprise an independently selected epoxide or optionally substituted, saturated or unsaturated cyclopropyl, cyclobutyl, cyclopentyl or cyclohexyl ring any of which rings optionally contain one or two independently selected —O—, —S—, —S(O)(O)—, —NH— —N(optionally substituted alkyl)- or ═N-heteroatoms;
R⁷ is —O—, —S—, —NR^{PR}, —C(R¹⁰)₂—, —C(R¹⁰)₂—C(R¹⁰)₂—, —C(R¹⁰)₂—C(R¹⁰)₂—C(R¹⁰)₂—, —C(R¹⁰)₂—O—C(R¹⁰)₂—, —C(R¹⁰)₂—S—C(R¹⁰)₂—, —C(R¹⁰)₂—NR^{PR}—C(R¹⁰)₂—, —O—C(R¹⁰)₂—, —S—C(R¹⁰)₂— or —NR^{PR}—C(R¹⁰)₂, where each R¹⁰ is independently selected;
R⁸ and R⁹ independently are —C(R¹⁰)₂—, —C(R¹⁰)₂—C(R¹⁰)₂—, —O—, —O—C(R¹⁰)₂—, —S—, —S—C(R¹⁰)₂—, —NR^{PR}— or —NR^{PR}—C(R¹⁰)₂—, or one or both of R⁸ or R⁹ independently are absent, leaving a 5-membered ring, where each R¹⁰ is independently selected;
R¹¹ is —O—, —S—, —S(O)(O)—, —NR^{PR}—, —CH₂—, —CHR¹⁰—, —C(R¹⁰)₂—, —C(R¹⁰)₂—O—C(R¹⁰)₂—, —C(R¹⁰)₂—S—C(R¹⁰)₂—, —C(R¹⁰)₂—S(O)(O)—C(R¹⁰)₂—, —C(R¹⁰)₂—NR^{PR}—C(R¹⁰)₂—, —O—C(R¹⁰)₂—, —S—C(R¹⁰)₂—, —S(O)(O)—C(R¹⁰)₂— or —NR^{PR}—C(R¹⁰)₂—, where each R¹⁰ is independently selected;
R¹³ independently is C₁₋₆ alkyl; and
R^{PR} independently are —H or a protecting group, wherein one or two independently selected R¹⁰ moieties are present at the 1-, 6- and 12-positions, optionally wherein the formula 1 compound is administered once per day daily or every other day for 1 to about 10 days and optionally wherein the formula 1 compound is administered in a formulation that comprises one, two or more excipients, wherein the formulation optionally is a solid or liquid oral formulation or a parenteral aqueous or non-aqueous formulation.

6. The method of claim 5 wherein the whole body radiation dose is about 600 cGy to about 635 cGy and the non-human primate is a rhesus monkey and optionally wherein the formula 1 compound is administered once per day for 1, 2, 3, 4, 5, 6, 7, 8, 9 or 10 consecutive days.

7. The method of claim 6 wherein the formula 1 compound has the structure 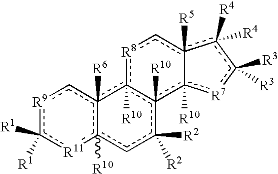
optionally wherein no double bond is present at the 17-position, R⁴ in the β-configuration is an O—, S— or N-linked moiety optionally selected from an ester, a thioether and a carmabate, R⁴ in the α-configuration is —H or a C-linked moiety optionally selected from optionally substituted alkyl and optionally substituted alkynyl, one R¹ is an O—, S— or N-linked moiety optionally selected from an ester, a thioether and a carmabate and the other R¹ is —H or a C-linked moiety optionally selected from optionally substituted alkyl and optionally substituted alkynyl or one R¹ is absent if a double bond is present at the 3-position and the remaining R¹ is an O—, S— or N-linked moiety, R² independently are —H or optionally substituted alkyl, R³ independently are —H, —F, —Cl, —Br, —I, optionally substituted alkyl or an O-linked moiety optionally selected from —OH, ═O and a carbonate, R⁵ is optionally substituted alkyl or is absent if a double bond is present at the 13-position, R⁶ is —H, optionally substituted alkyl or is absent if a double bond is present at the 13-position and R¹⁰ independently are —H, —F, —OH, optionally substituted alkyl or absent if a double bond is present at the ring carbon to which the R¹⁰ moiety is bonded.

8. A method to identify a treatment method useful to increase the rate or probability of survival of an injured human or non-human primate, comprising
(1) exposing non-human primates to a biological insult of at least about an LD_{40/30} to obtain exposed subjects and conducting a treatment protocol obtain exposed treated subjects, wherein the exposed treated subjects are not provided with an ameliorative treatment selected from (i) a transfusion such as a whole blood transfusion(s), a platelet transfusion(s), or an immunoglobulin transfusion(s), (ii) an antimicrobial treatment(s) to treat or prevent an infection, (iii) assisted feeding such as feeding by parenteral or catheter feeding or by tube feeding to the stomach; and
(2) determining the survival rate of the exposed treated subjects to obtain a treatment survival rate and comparing the treatment survival rate with a suitable control survival rate that was obtained from exposed subjects that were not provided with the ameliorative treatment, but that were treated with about 1 mg/kg/day to about 45 mg/kg/day of androst-5-ene-3β,17β-diol, 17α-methylandrost-5-ene-3β,17β-diol, 17α-ethynylandrost-5-ene-3β,17β-diol, 16α-fluoroandrost-5-ene-3β,17β-diol, 16α-fluoroandrost-5-ene-3α,17β-diol, 16α-fluoroandrost-5-ene-3β,17α-diol, 16α-fluoroandrost-5-ene-3α,17α-diol, 16α-fluoro-17α-methylandrost-5-ene-3β,17β-diol, 16α-fluoro-17α-ethynylandrost-5-ene-3β,17β-diol, 16α-fluoroandrost-5-ene-17β-ol or 16α-fluoroandrost-5-ene-17α-ol or a 2-oxa, 4-ene, 5α-androstane, 5β-androstane and/or 19-nor analog of any of these compounds.

9. The method of claim 8 wherein the biological insult is exposure of the non-human primates to whole body radiation.

10. The method of claim 9 wherein the whole body radiation dose is about 600 cGy to about 635 cGy and the non-human primate is a rhesus monkey.

11. The method of claim 9 wherein the treatment protocol is administered beginning at about 21 days before through about 4 days after the exposure of the non-human primates to the whole body radiation.

12. The method of claim 9 wherein the treatment protocol comprises administering about 0.1 mg/kg/day to about 60 mg/kg/day of a formula 1 compound having the structure 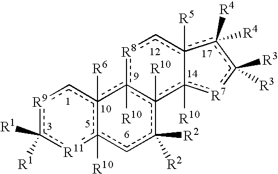
wherein the dotted lines are optional double bonds and 0, 1, 2, 3, 4 or 5 double bonds are present;
each R¹, R², R³, R⁴, R⁵, R⁶ and R¹⁰ independently or together are —H, —OH, —OR^{PR}, —SR^{PR}, —SH, —N(R^{PR})₂, —NHR^{PR}, —NH₂, —O—Si—(R¹³)₃, —CHO, —CHS, —CN, —SCN, —NO₂, —N₃, —COOH, —COOR^{PR}, —OSO₃H, —OSO₂H, —OPO₃H₂, ═O, ═S, ═N—OH, ═N—OCH₃, ═CH₂, ═CH—CH₃, ═CH-optionally substituted alkyl, ═N-optionally substituted alkyl, ═N—O-optionally substituted alkyl, —NH—S(O)(O)-optionally substituted alkyl, —S—S-optionally substituted alkyl, ester, thioester, thionoester, phosphoester, phosphothioester, phosphonate, phosphonate ester, thiophosphonate, thiophosphonate ester, phosphiniester, sulfite ester, sulfate ester, sulfamate, sulfonate, sulfonamide, amide, amino acid, peptide, ether, thioether, acyl, thioacyl, carbonate, carbamate, halogen, optionally substituted alkyl, optionally substituted alkenyl, optionally substituted alkynyl, optionally substituted aryl, optionally substituted heteroaryl, optionally substituted heterocycle, optionally substituted monosaccharide, optionally substituted oligosaccharide, polymer, spiro ring, epoxide, acetal, thioacetal, ketal or a thioketal, ═N—O-optionally substituted alkyl, ═N-optionally substituted alkyl, —NH-optionally substituted alkyl, —N(optionally substituted alkyl)₂ where each optionally substituted alkyl is independently selected, or, one or more of two adjacent R¹, R², R³, R⁴, R⁵, R⁶ and R¹⁰ comprise an independently selected epoxide or optionally substituted, saturated or unsaturated cyclopropyl, cyclobutyl, cyclopentyl or cyclohexyl ring any of which rings optionally contain one or two independently selected —O—, —S—, —S(O)(O)—, —NH— —N(optionally substituted alkyl)- or ═N-heteroatoms;
R⁷ is —O—, —S—, —NR^{PR}—, —C(R¹⁰)₂—, —C(R¹⁰)₂—C(R¹⁰)₂—, —C(R¹⁰)₂—C(R¹⁰)₂—C(R¹⁰)₂—, —C(R¹⁰)₂—O—C(R¹⁰)₂—, —C(R¹⁰)₂—S—C(R¹⁰)₂—, —C(R¹⁰)₂—NR^{PR}—C(R¹⁰)₂—, —O—C(R¹⁰)₂—, —S—C(R¹⁰)₂— or —NR^{PR}—C(R¹⁰)₂—, where each R¹⁰ is independently selected;
R⁸ and R⁹ independently are —C(R¹⁰)₂—, —C(R¹⁰)₂—C(R¹⁰)₂—, —O—, —O—C(R¹⁰)₂—, —S—, —S—C(R¹⁰)₂—, —NR^{PR}— or —NR^{PR}—C(R¹⁰)₂—, or one or both of R⁸ or R⁹ independently are absent, leaving a 5-membered ring, where each R¹⁰ is independently selected;
R¹¹ is —O—, —S—, —S(O)(O)—, —NR^{PR}, —CH₂—, —CHR¹⁰—, —C(R¹⁰)₂—, —C(R¹⁰)₂—O—C(R¹⁰)₂—, —C(R¹⁰)₂—S—C(R¹⁰)₂—, —C(R¹⁰)₂—S(O)(O)—C(R¹⁰)₂—, —C(R¹⁰)₂—NR^{PR}—C(R¹⁰)₂—, —O—C(R¹⁰)₂—, —S—C(R¹⁰)₂—, —S(O)(O)—C(R¹⁰)₂— or —NR^{PR}—C(R¹⁰)₂—, where each R¹⁰ is independently selected;
R¹³ independently is C₁₋₆ alkyl; and
R^{PR} independently are —H or a protecting group, wherein one or two independently selected R¹⁰ moieties are present at the 1-, 6- and 12-positions, optionally wherein the formula 1 compound is administered once per day daily or every other day for 1 to about 10 days.

13. The method of claim 12 wherein the whole body radiation dose is about 600 cGy to about 635 cGy and the non-human primate is a rhesus monkey.

14. The method of claim 13 wherein the formula 1 compound has the structure 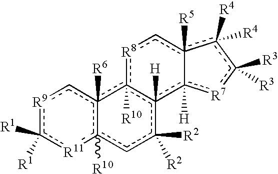
optionally wherein no double bond is present at the 17-position, R⁴ in the β-configuration is an O—, S— or N-linked moiety optionally selected from an ester, a thioether and a carmabate, R⁴ in the a-configuration is —H or a C-linked moiety optionally selected from optionally substituted alkyl and optionally substituted alkynyl, one R¹ is an O—, S— or N-linked moiety optionally selected from an ester, a thioether and a carmabate and the other R¹ is —H or a C-linked moiety optionally selected from optionally substituted alkyl and optionally substituted alkynyl or one R¹ is absent if a double bond is present at the 3-position and the remaining R¹ is an O—, S— or N-linked moiety, R² independently are —H or optionally substituted alkyl, R³ independently are —H, —F, —Cl, —Br, —I, optionally substituted alkyl or an O-linked moiety optionally selected from —OH, ═O and a carbonate, R⁵ is optionally substituted alkyl or is absent if a double bond is present at the 13-position, R⁶ is —H, optionally substituted alkyl or is absent if a double bond is present at the 13-position and R¹⁰ independently are —H or —F or absent if a double bond is present at the ring carbon to which the R¹⁰ moiety is bonded.

15. A method to determine a status profile for a subject species comprising,
(1) exposing subjects to a biological insult of at least about an LD_{40/30} to obtain exposed treated subjects;
(2) measuring on two or more occasions in or from the exposed subjects one, two or more biological parameters selected from temperature, circadian rhythm, red blood cell counts, hematocrit, reticulocytes, platelets, megakaryocytes and neutrophils;
(3) measuring the survival rate of the exposed subjects;
(4) obtaining one or more status profiles that corresponds to a defined probability of surviving the biological insult (Pₛᵤᵣᵥᵢᵥₐₗ) of at least 0.95 or of not surviving the biological insult (P_{lethality}) of at least 0.05; and
(5) optionally using the status profile to identify and initiate a profile-based therapy for one or more of the exposed subjects.

16. The method of claim 15 wherein the biological insult is about an LD_{30/60} to about an LD_{70/60} or wherein the biological insult is about an LD_{50/60} and the subject species is a non-human primate.

17. The method of claim 16 wherein the temperature is core body temperature, which is optionally measured (i) using an implanted monitor, and/or (ii) continuously and/or (iii) at intervals of about 1 minute, about 5 minutes or about 10 minutes to about 30 minutes, about 1 hour or about 2 hours.

18. The method of claim 16 wherein the biological insult is a radiation exposure, optionally selected from γ-radiation, X-rays, β-radiation, fast neutrons and slow neutrons.

19. The method of claim 16 wherein the profile-based therapy is administration of an effective amount of an apoptosis inhibitor, α-1 thymosin, a hematopoiesis stimulator, an immune system stimulator optionally selected from one or more of G-CSF, GM-CSF, TPO, EPO, stem cell factor, pegfilgrastim and a formula 1 compound of claim 2.

20. A method comprising,
(a) administering a radiation dose of (i) about 580 cGy to about 640 cGy or (ii) about an LD_{30/30} to about an LD_{90/30}, to a non-human primate(s) and
(b) administering a test treatment compound or protocol to the irridiated non-human primate(s), wherein the irridiated non-human primate(s) are not provided with an ameliorative treatment selected from (i) a transfusion such as a whole blood transfusion(s), a platelet transfusion(s), or an immunoglobulin transfusion(s), (ii) an antimicrobial treatment(s) to treat or prevent an infection, (iii) assisted feeding such as feeding by parenteral or catheter feeding or by tube feeding to the stomach.

21. The method of claim 20 wherein the test treatment compound or protocol is administration of about 5 mg/kg/day to about 45 mg/kg/day of a formula 1 compound of claim 2 or 3β,17β-dihydroxyandrost-5-ene, optionally wherein the a formula 1 compound of claim 2 or the 3β,17β-dihydroxyandrost-5-ene is administered once per day or every 2^{nd} day or every 3^{rd} day for 1, 2, 3, 4, 5, 6, 7, 8, 9, 10, 11, 12, 13, 14, 15, 16, 17 or 18 days beginning within about 0.5-12 hours after the irridiation of the irridiated non-human primate(s). The method of claim 20 wherein the test treatment compound or protocol is administration of about 5 mg/kg/day to about 45 mg/kg/day of a formula 1 compound of claim 2 or 3β,17β-dihydroxyandrost-5-ene, optionally wherein the a formula 1 compound of claim 2 or the 3β,17β-dihydroxyandrost-5-ene is administered once per day or every 2^{nd} day or every 3^{rd} day for 1, 2, 3, 4, 5, 6, 7, 8, 9 or 10 days beginning within about 2-4 hours after the irridiation of the irridiated non-human primate(s).

22. The method of claim 21 further comprising determining the survival rate of the exposed treated subjects to obtain a treatment survival rate, and optionally comparing the treatment survival rate with a suitable control survival rate that was obtained from exposed subjects that were not provided with any treatment protocol and that were not provided with the ameliorative treatment.

23. The method of claim 22 wherein the radiation dose is whole body radiation, optionally wherein the dose is (i) about 600 cGy to about 635 cGy or (ii) about an LD_{35/30} to about an LD_{80/30}.

24. The method of claim 23 wherein the test treatment compound or protocol is administration of a F1C, optionally wherein the F1C is androst-5-ene-3β,17β-diol or wherein the F1C is a 1,5,9(11)-triene, 1,5,11-triene, 1,5,14-triene, 1,5,15-triene, 1,3,14-triene, 1,3,8(9)-triene, 1,3,8(14)-triene, 1,3,9(11)-triene, 1,3,11-triene or a 1,3,14-triene, optionally wherein one R¹ is an O-linked moiety or an N-linked moiety, the other R¹is —H or a C-linked moiety, or, if a double bond is present at the 3-position, R¹ is O-linked moiety or an N-linked moiety, R⁴ in the β-configuration is an O-linked moiety, R⁴ in the α-configuration is —H or a C-linked moiety and one or both R³ independently or together are —H, —OH, ═O, an O-linked moiety, halogen or a C-linked moiety and optionally wherein about 5 mg/kg/day to about 45 mg/kg/day of the formula 1 compound of claim 2 or the 3β,17β-dihydroxyandrost-5-ene is administered once per day on 1, 2, 3, 4, 5 or more days.

25. The method of claim 23 wherein the survival rate of the exposed treated subjects is significantly increased compared to suitable control irridiated non-human primate(s) that were not treated with the test treatment compound or protocol or wherein the survival rate of the exposed treated subjects is increased by about 5%, about 10%, about 15%, about 20%, about 25%, about 30% or more compared to suitable control suitable control irridiated non-human primate(s).

26. A method to (A) maintain approximately normal endogenous levels of 3β,17β-dihydroxyandrost-5-ene and/or 17β-hydroxyandrost-5-ene-3β-sufate in a human or non-human primate that has been or that is anticipated to be exposed to (i) a biological insult of at least about an LD₁₀, (ii) a cytotoxic chemotherapy or (iii) a radiation exposure or (B) decrease the drop in endogenous levels of 3β,17β-dihydroxyandrost-5-ene and/or 17β-hydroxyandrost-5-ene-3β-sufate in human or non-human primate, comprising, administering to the human or non-human primate an effective amount of a modulator compound selected from a steroid hydroxylase inhibitor, a 3-hydroxysteroid dehydrogenase inhibitor, a 17-hydroxysteroid dehydrogenase inhibitor, a steroid sulfotransferase inhibitor and an isoform or isozyme of any of these enzymes.

27. The method of claim 26 wherein
(a) the 17-hydroxysteroid dehydrogenase is a 17β-hydroxysteroid dehydrogenase optionally selected from type 1, 2, 3, 4, 5, 7, 8 or 10 human 17β-hydroxysteroid dehydrogenase or a non-human primate homolog or oxidative isoform of any of these enzymes; or
(b) the 3-hydroxysteroid dehydrogenase is a 3β-hydroxysteroid dehydrogenase optionally selected from type 1, 2, 3, 4 or 5 human 3β-hydroxysteroid dehydrogenase or a non-human primate homolog or oxidative isoform of any of these enzymes; or
(c) the 3-hydroxysteroid dehydrogenase is a 3α-hydroxysteroid dehydrogenase or a non-human primate homolog of any of a 3α-hydroxysteroid dehydrogenase; or
(d) the sulfotransferase is a human sulfotransferase optionally selected from type 1 or 2 human sulfotransferase or a non-human primate homolog of any of these enzymes; or
(e) the steroid hydroxylase is a 2-hydroxylase, a 7-hydroxylase, an 11-hydroxylase or an 15-hydroxylase, optionally selected from a human 2α-hydroxylase, a human 7α-hydroxylase, a human 6β-hydroxylase, a human 7β-hydroxylase, a human 15α-hydroxylase, an 11α-hydroxylase or an 11β-hydroxylase optionally selected from a human steroid CYP3A hydroxylase, a human steroid CYP2A hydroxylase, a human steroid CYP7B hydroxylase, a human steroid CYP2B hydroxylase or an isoform or homolog such as CYP3A1, CYP3A4, CYP3A4*1 B, CYP3A9, CYP3A23, CYP3A27, CYP3A45, CYP2A2, CYP1A2, CYP2A, CYP2A4, CYP2A5, CYP2A8, CYP2B, CYP2C, CYP2G1, CYP3A, CYP2A13, CYP2A5, CYP2A6 or a human or non-human primate homolog of any of these enzymes.
